(19) United States
(12) Patent Application Publication
(10) Pub. No.: US 2025/0361525 A1
(43) Pub. Date: Nov. 27, 2025

(54) AAV VECTORS FOR GENE EDITING

(71) Applicant: Scribe Therapeutics Inc., Alameda, CA (US)

(72) Inventors: Fred DEITER, Alameda, CA (US); Wenyuan ZHOU, Dublin, CA (US); Katherine BANEY, Berkeley, CA (US); Isabel COLIN, Oakland, CA (US); Cécile FORTUNY, Oakland, CA (US); Addison WRIGHT, El Cerrito, CA (US); Brett T. STAAHL, Tiburon, CA (US); Sean HIGGINS, Alameda, CA (US); Benjamin OAKES, El Cerrito, CA (US); Suraj MAKHIJA, San Francisco, CA (US); Sarah DENNY, San Francisco, CA (US); Manuel MOHR, Berkeley, CA (US); Torrey BROWNELL, Oakland, CA (US); Angus SIDORE, Oakland, CA (US)

(21) Appl. No.: 18/872,584

(22) PCT Filed: Jun. 7, 2023
(86) PCT No.: PCT/US2023/068097

§ 371 Date: Dec. 6, 2024

Related U.S. Application Data
(60) Provisional application No. 63/350,376, filed on Jun. 8, 2022.

(51) Int. Cl. C12N 15/86; A61K 48/00; C12N 9/22; C12N 15/11; C12N 15/90

(52) U.S. Cl. CPC C12N 15/86; A61K 48/0041; C12N 9/226; C12N 15/11; C12N 15/907; C12N 2310/20; C12N 2750/14144; C12N 2750/14152

(57) ABSTRACT

Provided herein are recombinant adeno-associated virus (rAAV) compositions and methods for use of the rAAV encoding CasX proteins and guide ribonucleic acid (gRNA) sequences useful for nucleic acid sequence editing, and including transgene components. The rAAV may be delivered to cells to target a gene of interest.

### CROSS-REFERENCE TO RELATED APPLICATIONS

This application claims priority to U.S. Provisional Patent Application No. 63/350,376, filed on Jun. 8, 2022, the contents of which are incorporated by reference in their entirety herein.

### INCORPORATION BY REFERENCE OF SEQUENCE LISTING

The contents of the electronic sequence listing (SCRB_044_01WO_SeqList_ST26.xml; Size: 14,517,322 bytes; and Date of Creation: Jun. 6, 2023) are herein incorporated by reference in its entirety.

### BACKGROUND

Gene editing holds great promise for treating or preventing many genetic diseases. However, safe and targeted delivery of CRISPR gene editing machinery into the desired cells is necessary to achieve therapeutic benefit. There remains a need in the art for compositions and methods for delivering CRISPR gene editing machinery to cells in vitro and/or in vivo.

### SUMMARY

The present disclosure relates to recombinant adeno-associated virus vectors (rAAV) for the delivery of Class 2, Type V CRISPR proteins and guide nucleic acids to cells for the modification of target nucleic acids.

In some embodiments, the present disclosure provides rAAV transgenes and transgene plasmids, as well as methods for the production of rAAV encoding the Class 2, Type V CRISPR proteins and guide ribonucleic acids (gRNA). In particular embodiments, the rAAV encode CasX nucleases and gRNA. In an advantage of the Type V systems, particularly the CasX system, the smaller size of the encoding sequences, relative to Cas9, permits the inclusion of encoding sequences for complete nuclease and multiple gRNA components, as well as promoters, accessory elements, or other useful payloads in the transgene that permit the formation of functional rAAV particles for transduction of target cells and the expression of the encoded CRISPR components. In some embodiments, the present disclosure provides rAAV comprising a first and a second gRNA wherein the first and/or the second gRNA comprise targeting sequences complementary to different or overlapping regions of a target DNA sequence. The rAAV are useful in a variety of methods for modification of target nucleic acids and in the treatment of diseases and disorders where modification of a gene can lead to amelioration or prevention of the disease or disorder.

In some embodiments, the present disclosure provides a method for treating a disease in a subject (e.g., a human) caused by one or more mutations in a gene of the subject, comprising administering a therapeutically effective dose of the rAAV of any of the embodiments disclosed herein.

In some embodiments, the present disclosure provides a method of reducing the immunogenicity of AAV vector components, comprising deleting all or a portion of the CpG dinucleotides of the sequences of the AAV components selected from the group consisting of 5′ ITR, 3′ ITR, Pol III promoter, Pol II promoter, encoding sequence for CRISPR nuclease, encoding sequence for gRNA, accessory element, and poly(A) signal sequences.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic of the AAV construct described in Example 1.

FIG. 2 shows results of an editing assay using AAV transgene plasmids nucleofected into mNPCs, as described in Example 1, demonstrating that the CasX and targeting guide in three different vectors (constructs 1, 2, and 3) edits on target (tdTomato) with high efficiency compared to non-targeting control (NT). Editing was assessed by FACS 5 days post-transfection. Data are presented as mean±SEM for n=3 replicates.

FIG. 3 shows results of an editing assay using AAV transgene plasmids nucleofected into mNPCs at four different dose levels, as described in Example 1. CasX delivered as an AAV transgene plasmid to mNPCs edits on target with high efficiency in a dose-dependent manner, compared to non-targeting control (NT). CasX variant 491 with gRNA scaffold 174 (gRNA scaffolds are also referred to herein gRNA variants, guide scaffolds) and spacer targeting tdTomato in three different vectors (constructs 1, 2, and 3) were nucleofected in mNPCs, and editing was assessed by FACS 5 days post-transfection. Data are presented as mean±SEM for n=3 replicates. #

FIG. 4 shows results of an editing assay using AAV vector construct 3 transduced into mNPCs at 3-fold dilutions, assessed by FACS five days post-transduction, as described in Example 1. Data are presented as mean±SEM for n=3 replicates. MOI. multiplicity of infection.

FIG. 5 is a scanning transmission micrograph showing AAV particles with packaged CasX variant 438, gRNA scaffold 174 and spacer 12.7, as described in Example 2. AAV were negatively stained with 1% uranyl acetate. Empty particles are identified by a dark electron dense circle at the center of the capsid.

FIG. 6 shows results of an immunohistochemistry staining of mouse coronal brain sections, as described in Example 3. Mice received an ICV injection of 1×10¹¹ AAV packaged with CasX 491, gRNA scaffold 174 with spacer 12.7 (top panel), which were able to edit the tdTom locus in the Ai9 mice (edited cells appear white). The bottom panel shows that CasX 491 and gRNA scaffold 174 with a non-targeting spacer administered as an AAV ICV injection did not edit at the tdTom locus. Tissues were processed for immunohistochemical analysis 1 month post-injection.

FIG. 7 shows the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 8 shows the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 9 shows the results of an editing assay of the tdTom locus in mNPCs using AAV transgene plasmids of constructs having variations in the CasX promoters and transgene size (see table insert), as described in Example 4. Editing was assessed by FACS 5 days post-transfection. Data are presented as mean±SEM for n=3 replicates.

FIG. 10 shows the results of an editing assay of the tdTom locus in mNPCs using AAV vectors incorporating the same promoters as shown in FIG. 9, as described in Example 4. The graph on the left are results testing 3-fold dilutions of the constructs, while the graph on the right are results of editing using an MOI of 2×10⁵ vg/cell. Editing was assessed by FACS 5 days post-transfection. Data are presented as mean±SEM for n=3 replicates.

FIG. 11 shows the results of an editing assay of the tdTom locus in mNPCs using AAV vectors with protein promoter variants designed to reduce transgene size, compared to AAV with the top 4 protein promoter variants identified previously (AAV.3, AAV.4, AAV.5 and AAV.6), as described in Example 4. Editing was assessed by FACS 5 days post-transfection. Data are presented as mean±SEM for n=3 replicates. The dashed line shows editing levels of AAV.4, the AAV construct that in this experiment was used as a baseline for comparison across the variants.

FIG. 12 is a graph of percent editing versus transgene size for all constructs having varying promoters tested in this study. Constructs circled with dashes were identified as having above average editing while minimizing transgene size. The dashed line shows editing levels of AAV.4, the AAV construct that in this experiment was used as a baseline for comparison across variants.

FIG. 13 shows the results of an editing assay of mNPCs using AAV transgene plasmids having variations in gRNA promoter strength, as described in Example 5. Editing was assessed by FACS 5 days post-transfection. Data are presented as mean±SEM for n=3 replicates.

FIG. 14 shows the results of an editing assay of mNPCs using three different AAV vectors having variations in gRNA promoter strength, as described in Example 5. The graph on the left are results testing 3-fold dilutions of the constructs ranging from 1×10⁴ to 5×10⁵ vg/cell, while the graph on the right are results of editing using an MOI of 3×10⁵ vg/cell. Editing was assessed by FACS 5 days post-transfection. Data are presented as mean±SEM for n=3 replicates.

FIG. 15 is a bar graph that shows percent editing of the tdTom locus in mNPCs in an experiment to assess use of truncated U6 RNA promoters in constructs when delivered in AAV transgene plasmids designed to minimize the footprint of the Pol III promoter in the delivered transgene, as described in Example 5. Editing was assessed by FACS 5 days post-transfection. Data are presented as mean±SEM for n=3 replicates.

FIG. 16 is a bar graph that shows percent editing of the tdTom locus in mNPCs comparing base construct 53 to construct 85, when delivered as AAV vector designed to minimize the footprint of the Pol III promoter in the delivered transgene, as described in Example 5.

FIG. 17 is a bar graph that shows editing results of the tdTom locus in an experiment to assess the effects of constructs having engineered U6 RNA promoters when delivered to mNPCs in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 18 is a scatter plot depicting transgene size of all AAV variants tested having engineered U6 RNA promoters on the X-axis vs. percent of mNPCs edited on the Y-axis, as described in Example 5. The dashed line indicates construct 53, having the largest promoter tested, while the dotted line indicates construct 89, having the smallest promoter tested.

FIG. 19 shows the results of an editing assay of the tdTom locus in mNPCs in an experiment to assess the effects of constructs having engineered Pol III RNA promoters when delivered in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 20 is a bar graph showing AAV-mediated editing level in mNPCs at an MOI of 3.0E+5 vg/cell using the indicated constructs, as described in Example 5.

FIG. 21 is a scatter plot depicting the transgene size (inclusive of ITRs) of all variants tested on the X-axis vs. the percent of mNPCs edited on the Y-axis, as described in Example 5.

FIG. 22 shows the results of an editing assay of the tdTom locus in mNPCs using AAV transgene plasmids having variations in poly(A) signals, as described in Example 6. Data are presented as mean±SEM for n=3 replicates.

FIG. 23 shows the results of an editing assay of the tdTom locus in mNPCs using two AAV vectors having the top poly(A) signals, as described in Example 6. Editing was assessed by FACS 5 days post-transfection. Data are presented as mean±SEM for n=3 replicates.

FIG. 24 is a graph plotting the RNA abundance ratio, determined as log 2(cDNA reads/viral DNA input reads) calculated across ten summed technical replicates per unique poly(A) library member assessed during the high-throughput screen, as described in Example 6. The depicted data were for one biological replicate. The bGH poly(A) signal sequence is highlighted as a positive control.

FIG. 25 are schematics of AAV plasmid constructs containing guide RNA transcriptional units (gRNA scaffold-spacer stack driven by a U6 promoter) in different orientations in regards to the protein promoter transcriptional unit, as described in Example 7. The tapered points depicts the orientation of the transcriptional unit for protein or guide RNA.

FIG. 26 shows the results of an editing assay of the tdTom locus in mNPCs using AAV transgene plasmids having differences in regulatory element orientation, as described in Example 7. Editing was assessed by FACS 5 days post-transfection. Data are presented as mean±SEM for n=3 replicates.

FIG. 27 shows the results of an editing assay of NPCs using AAV vectors containing guide RNA transcriptional units (gRNA scaffold-spacer stack driven by a U6 promoter) in different orientations in relation to the protein promoter transcriptional unit, as described in Example 7. The graph on the left shows results testing 3-fold dilutions of the constructs ranging from 1×10⁴ to 2×10⁶ vg/cell. The bar graph on the right shows AAV-mediated percent editing in mNPCs at an MOI of 3.0E+5 vg/cell. Editing was assessed by FACS 5 days post-transfection. Data are presented as mean±SEM for n=3 replicates.

FIG. 28 illustrates the schematics of AAV plasmid constructs containing various configurations of the gRNA transcriptional unit (Pol III U6 promoter driving the expression of the gRNA scaffold and indicated spacer) as described in Example 7.

FIG. 29 is a graph showing the quantification of percent editing at the tdTomato locus in mNPCs 5 days post-transduction with AAVs produced from the indicated AAV constructs, as described in Example 7. Editing was assessed by FACS five days post-transduction.

FIG. 30 is a bar graph of results of an editing assay of the tdTom locus in mNPCs using AAV transgene plasmid constructs having different post-transcriptional regulatory elements compared to constructs not having post-transcriptional regulatory elements, as described in Example 8. Editing was assessed by FACS 5 days post-transfection. Data are presented as mean±SEM for n=3 replicates.

FIG. 31 is bar graph showing AAV-mediated editing levels (grey bars) of mNPCs at a viral MOI of 3.0E+5 compared to nucleofection editing using 150 ng of AAV-cis plasmids (dark bars) expressing the CasX protein 491 under the control of top promoters without (constructs 4, 5, 6) or in combination with different post-transcriptional regulatory element sequences (constructs 35-37 for base plasmid 4, constructs 38-39 for base plasmid 5, and constructs 42-43 for base plasmid 6)., as described in Example 8. Editing was assessed by FACS 5 days post-transfection. Data are presented as mean±SEM for n=3 replicates.

FIG. 32 is a bar graph showing AAV-mediated editing levels of mNPCs at a viral MOI of 3.0E+5 for constructs under promoters without (constructs 58, 59, 53) or in combination of different post-transcriptional regulatory element sequences (respectively constructs 72-74 for base plasmid 58 containing Jet promoter, constructs 75-77 for base plasmid 59 containing Jet+USP promoter, and constructs 80-81 for base plasmid 53 containing UbC promoter), as described in Example 8. Editing was assessed by FACS 5 days post-transfection. Data (n=3) are presented as mean±SEM.

FIG. 33 is a scatterplot comparing the transgene size of each construct evaluated (from ITR to ITR, in bp) to AAV-mediated editing levels in mNPCs at a MOI of 3.0e+5 vg/cell, as described in Example 8. The circled data points represent the top identified constructs in terms of editing levels of select transgene size. The horizontal grey line shows the editing level of the benchmark vector AAV.53 for comparative purposes. The vertical grey line delimits vectors that are over or under a 4.9kb transgene size.

FIG. 34 is a violin plot displaying AAV-mediated fold-improvement from the inclusion of the indicated PTRE element in the transgene plasmid, relative to its base (transgene with same promoter but no PTRE, indicated by gray dashed line), as described in Example 8.

FIG. 35 is a bar chart showing editing results of constructs with different neuronal enhancers delivered as AAV transgene plasmids to mNPCs, as described in Example 8. The gray lines show editing levels of reference plasmid 64, harboring CMV enhancer+core promoter. Editing was assessed by FACS 5 days post-transfection. Data are presented as mean±SEM for n=3 replicates.

FIG. 36 depicts the results of an editing assay measured as indel rate detected by NGS at the DMPK 3′ UTR locus for the indicated AAV dual-guide systems transduced into HEK293T cells in a series of three-fold dilution, as described in Example 9.

FIG. 37 is a bar chart displaying the breakdown of indels generated by type of editing (single edit at the 5′ or 3′ of CTG repeat or double-cut resulting in CTG repeat dropout) at the DMPK 3′ UTR locus by AAVs harboring the dual guide spacer combination (spacers 20.7 and 20.11), as described in Example 9. The percentage of single or dual-edits were calculated from the total percent of reads analyzed.

FIG. 38 shows schematics of AAV constructs with alternative gRNA configurations for constructs having multiple gRNA, as described in Example 9. The top schematic is architecture 1, while the bottom is architecture 2. The tapered points depict the orientation of the transcriptional unit for protein or guide RNA.

FIG. 39 shows schematics of AAV constructs with alternative gRNA configurations for constructs having multiple gRNA, as described in Example 9. The tapered points depict the orientation of the transcriptional unit for protein or guide RNA.

FIG. 40 shows schematics of guide RNA stack (Pol III promoter, scaffold, spacer) architectures tested with nucleofection and AAV transduction, as described in Example 9. Transgene harbors dual stacks in different orientations, with spacer 12.7, 12.2 and non-target spacer NT. The tapered points depict the orientation of the transcriptional unit for protein or guide RNA.

FIG. 41 shows the results of an editing assay for constructs having guide RNA stacks delivered via plasmid transfection to mNPCs, showing constructs with RNA stacks edit with enhanced potency compared to non-targeting control (NT), as described in Example 9. Editing was assessed by FACS 5 days post-transfection. Data are presented as mean±SEM for n=3 replicates.

FIG. 42 shows the results of an editing assay of mNPCs using AAV transgene plasmid constructs having multiple gRNA in different architectures and with different combinations of spacers (see FIG. 35) compared to construct 3 having a single gRNA and to a non-targeting construct, as described in Example 9. Editing was assessed by FACS 5 days post-transfection. Data are presented as mean±SEM for n=3 replicates.

FIG. 43 shows the results of an editing assay of mNPCs using AAV vector constructs 45-48 having multiple gRNA in different architectures and with different combinations of spacers (see FIG. 35) compared to construct 3, as described in Example 9. The left panel shows editing results using 3-fold MOI dilutions ranging from 1×10⁴ to 3×10⁵ vg/cell, while the right panel shows editing results at an MOI of 3×10⁵ vg/cell. Editing was assessed by FACS 5 days post-transfection. Data are presented as mean±SEM for n=3 replicates.

FIG. 44 is a bar graph of percent editing in mNPCs using AAV transgene plasmid constructs with varying 5′ NLS combinations (2, 7, and 9 in Table 20) with 3′ NLS 1, 8 and 9 in mNPCs, as described in Example 10.

FIG. 45 is a bar graph of percent editing in mNPCs using AAV vectors with varying 5′ NLS combinations with 3′ NLS 1, 8 and 9 in mNPCs, as described in Example 10.

FIG. 46 is a bar graph of percent editing in mNPCs using AAV vectors with varying NLS combinations when delivered in a vector designed to minimize the footprint of Pol III promoter in the transgene.

FIG. 47 is a schematic showing the organization of the components of an exemplary AAV transgene between the 5′ and 3′ ITRs, as described in Example 12.

FIG. 48A show results of editing assays in mNPCs nucleofected with 1000 of AAV-cis plasmids expressing CasX protein 491 expression of CMV and gRNA scaffolds 174 and 229-237 with spacer 11.30 targeting the mouse RHO exon 1 locus demonstrating improved activity at mouse RHO exon 1 in a dose-dependent manner, as described in Example 12. Triplicate wells were pooled together for gDNA extraction and therefore treated as n=1.

FIG. 48B is a bar graph showing fold-change in editing levels for each engineered gRNA scaffolds (229-237) relative to gRNA scaffold174 with spacer 11.30 (set to a value of 1.0) across two plasmid nucleofection doses 1000 and 500ng of AAV-cis plasmids, as described in Example 12. Triplicate wells were pooled together for gDNA extraction and therefore treated as n=1.

FIG. 49A show editing results of engineered gRNA scaffold 235 compared to gRNA scaffold 174 with spacer 11.1 targeting RHO at the exogenous RHO-GFP locus (with GFP as the reporter), under the expression of Pol III hU6 promote in ARPE-19 cells, demonstrating improved activity by the 235 variant at the human RHO locus, with increased on-target activity at WT exogenous RHO without off-target cleavage at the mutant RHO reporter gene, as described in Example 12. Data (n=3) are presented as mean±SD.

FIG. 49B is a bar graph displaying fold-change in editing levels of engineered gRNA scaffold 235 compared to gRNA scaffold 174 at the human RHO locus, with p59.491.235.11.1 normalized to benchmark p59.491.174.11.1 levels (set to value 1.0) in cells nucleofected with 1000 ng of each plasmid, as described in Example 12. Data (n=3) are presented as mean±SD.

FIG. 50A shows editing levels in mNPCs by AAV-mediated expression of CasX molecule and engineered gRNA scaffold 235 compared to gRNA scaffold 174 with spacer 11.30 at 3 different MOI levels, confirming increased editing levels at the endogenous mouse Rho exon 1 locus with no off-target locus, as described in Example 12.

FIG. 50B is a bar graph displaying fold-change in editing levels in mNPCs by AAV-mediated expression of CasX molecule and engineered guide variant 235 compared to gRNA scaffold 174 with spacer 11.30 in cells infected at a 5.0e+5 MOI, as described in Example 12. Data are presented as the mean of n=3.

FIG. 51A shows editing results at the human RHO locus in mNPCs nucleofected with 1000 and 500 ng of AAV-cis plasmids expressing CasX protein 491 and gRNA-scaffold 174 with on-target spacers of varying length, demonstrating improved on-target editing at the mouse RHO locus, as described in Example 12. Spacers variants are: 11.30 (20 nt WT RHO), 11.38 (18 nt WT RHO), and 11.39 (19 nt WT RHO), respectively. A control spacer, no-target (NT), designed to not recognize any sequence across the mouse and human genomes, was also tested as a negative control to ensure no unspecific targeting resulting from the expression of the CasX protein alone. Triplicate wells were pooled together for gDNA extraction and therefore treated as n=1.

FIG. 51B is a bar graph showing editing levels at the human RHO locus in nucleofected mNPCs with 1000 ng of AAV-cis plasmids expressing CasX protein 491 and gRNA-scaffold 174 with the indicated off-target spacers, as described in Example 12.

FIG. 51C is a bar graph displaying fold-change in editing levels at the human RHO locus in nucleofected mNPCs for each gRNA-scaffold 174 with spacer variants 11.38 and 11.39 normalized to levels of parental gRNA-scaffold-spacer 174.11.30, as described in Example 12. Data shows means+SD across 3 different biological replicates.

FIG. 52A is a Whisker box graph showing editing results of RHO in a mouse model comparing AAV-mediated delivery of gRNA scaffold variants and optimized spacers compared to benchmark construct, as described in Example 13. Each dot represents one retina (n=8-16). One-way ANOVA statistical test was performed, ***=p<0.001.

FIG. 52B is a Whisker box graph showing the relative fold-change in editing of RHO in a mouse model comparing AAV-mediated delivery of gRNA scaffold variants 174 and 235 and optimized spacers compared to benchmark construct, as described in Example 13. Values are relative to the benchmark vector AAV.RHO.174.11.30 (set to a value of 1). Each dot represents one retina (n=8-16).

FIG. 53A is a bar graph showing CTC-PAM editing levels (indel rates) at the mouse RHO locus in mNPCs nucleofected with 1000 and 500 ng of AAV-cis plasmids expressing the CasX protein variant 491, 515, 527, 528, 535, 536 or 537, respectively, and gRNA-scaffold 235.11.37 (on target), as described in Example 14. A control spacer, no-target (NT), designed to not recognize any sequence across the mouse and human genomes, was also tested as a negative control to ensure no unspecific targeting resulting from the expression of the CasX protein alone. Triplicate wells were pooled together for gDNA extraction and therefore treated as n=1.

FIG. 53B is a bar graph showing CTC-PAM editing levels (indel rates) at the mouse RHO locus in mNPCs nucleofected with AAV-cis plasmids expressing the CasX protein variant 491, 515, 527, 528, 535, 536 or 537, respectively, and gRNA-scaffold 235.11.39 (off-target), as described in Example 14.

FIG. 53C shows a bar graph displaying fold-change in editing levels for each indicated CasX protein variant with guide 235 and spacer 11.39, with results normalized to levels of the parental CasX protein 491, as described in Example 14.

FIG. 54A shows a bar graph showing editing levels in ARPE-19 mNPC nucleofected with 1000 ng of AAV-cis plasmids expressing CasX protein variant 491, 515, 527, 528, 535, 536 or 537 and guide variant 235 with spacer 11.41 or 11.43, as described in Example 14. Data (n=3) are presented as mean±SD.

FIG. 54B shows a bar graph displaying fold-change in editing levels in ARPE-19 mNPC nucleofected with 1000 ng of AAV-cis plasmids expressing CasX protein variant 515, 527, 528, 535, 536 or 537 and guide variant 235 with spacer 11.41 or 11.43 relative to benchmark p59.491.235.11.41 levels (set to a value of 1.0), as described in Example 14. Data (n=3) are presented as mean±SD.

FIG. 55A shows a bar graph of AAV-mediated editing levels in mNPCs at the endogenous mouse Rho exon 1 locus, as described in Example 14. mNPCs were infected using a 3.0e+5 and 1.0e+5 vg/cell MOI with AAV vectors expressing the indicated CasX protein 491, 515, 527, 528, 535, or 537 and gRNA-scaffold variant 235.11.39, as described in Example 14. Data (n=3) are presented as the mean.

FIG. 55B is a bar graph displaying fold-change in editing levels for the indicated CasX variant with gRNA scaffold 235 relative to gRNA scaffold 174 with spacer 11.39 in cells infected with the indicated MOI, as described in Example 14.

FIG. 56 is an illustration of reference mRHO exon 1 locus and target amino acid residue P23 (CCC) sequence (highlighted in bold), showing spacer 11.30 target sequence and expected CasX-mediated cleavage, as described in Example 15. The most common predicted edits quantified in CRISPResso edits (substitution/deletions) are displayed under the reference genome).

FIG. 57A shows results of in vivo AAV CasX-mediated editing of the mRHO P23 locus in retinae in C57BL6J mice (n=6-8; quantification in percent of total indels detected by NGS), as described in Example 15.

FIG. 57B shows the fraction (%) of AAV CasX-mediated frame-shift edits of the mRHO P23 locus in the retinae in C57BL6J (n=6-8) mice (n=6-8; quantification in percent of total indels detected by NGS), as described in Example 15.

FIGS. 58A-58F show representative fluorescence imaging of retinas from AAV-CasX treated mice or negative controls and stained, as described in Example 15. Cell nuclei were counterstained with DAPI (top row; FIGS. 58A-58C) to visualized retinal layers and stained with HA-tag (bottom row, FIGS. 58D-58F) antibody to detect CasX expression in photoreceptors (ONL) and other retinal layers (INL; GCL). Legends: ONL=Outer nuclear layer; INL=Inner nuclear layer, GCL=Ganglion cell layer.

FIG. 59A is a box plot showing median, minimal and highest editing values using AAV-mediated expression of CasX 491 detected by NGS 3 weeks post-injection in wild-type retinae injected with 5.0e+9 vg/eye of AAV.X.491.174.11.30 vectors, in which the 491 protein is driven by promoter variants designed to selectively express in rod photoreceptors (X=RP1−RP5) or a ubiquitous promoter (X=CMV), as described in Example 16. The grey line is placed at the editing levels achieved by AAV.RP1.491.174.11.30 to compare to other viral vectors tested.

FIG. 59B is a plot displaying levels of editing achieved by AAV vectors in wild-type retinae injected with 5.0e+9 vg/eye of AAV.X.491.174.11.30 vectors, compared to total transgene size (bp), as described in Example 16. The grey line delimitates transgenes below or above 4.9kb size.

FIG. 60 shows in vivo editing results that AAV-mediated expression of CasX 491 and gRNA spacer 174.4.76 in rod photoreceptors led to detectable levels of editing levels at integrated Nrl-GFP locus in a dose-dependent manner, as described in Example 16. The bar graph shows editing levels detected by NGS at the integrated GFP locus 4-weeks and 12-weeks post-injection in heterozygous Nrl-GFP mice injected with the indicated doses of AAV.RP1.491.174.4.76 vectors in one eye, and the vehicle control in the contralateral eye).

FIG. 61A shows a western blot of retinal lysates from positive (C1, uninjected homozygous Nrl-GFP retinae) and negative (N, uninjected C57BL/6J retinae) controls, vehicle groups (V, AAV formulation buffer injected retinae) and AAV-CasX 491, gRNA scaffold 174 and spacer 4.76 treated retinae with the medium dose 1.9e+9 (M) or high dose 1.0e+10 vg (H arm. Blots display the respective bands for the HA protein (CasX protein, top), GFP protein (middle) and GAPDH (bottom panels) used as a loading control, as described in Example 16. Levels of percent editing in the retinae detected by NGS are displayed under the blot for each sample.

FIG. 61B is a scatter boxplot representing levels of GFP protein detected in the western blots of FIG. 56A (ratios of densitometric values of the GFP band for total amount of proteins, normalized to the vehicle group levels), as described in Example 16. One-way ANOVA statistical analysis was performed (*=p<0.5).

FIG. 61C is a plot correlating GFP protein fraction to levels of editing achieved in mouse retinae of the AAV-treated mice, for both the 1.0e+9 and 1.0e+10 dose groups, as described in Example 16.

FIG. 62A is a bar graph representing the ratio of GFP fluorescence levels (superior to inferior retina mean grey values) detected by fundus imaging at 4-weeks compared to 12-weeks post-injection in mice injected with two dose levels of AAV constructs, as described in Example 16.

FIG. 62B displays representative images of fluorescence fundus imaging of GFP in retina from mice injected with 1.0e+9 vg (#13) or 1.0e+10vg (#34) with the AAV constructs at 4-weeks and (left panel) or 12-weeks (right panel), as described in Example 16.

FIGS. 63A-63L present histology images or retinae of mice stained with various immunochemistry reagents, as described in Example 16, confirming efficient knock-down of GFP in photoreceptor cells in an AAV-dose dependent manner. The images are representative confocal images of cross-sectioned retinae injected with vehicle (FIGS. 63A, 63B, 63C, 63D), AAV-CasX at a 1.0e+9 vg dose (FIGS. 63E, 63F, 63G, and 63H) and 1.0E+10vg dose (FIGS. 631, 63J, 63K, and 63L). Structural imaging shows GFP expression by rod photoreceptors in the outer segment (images in FIGS. 63A, 63E, 63I and images FIGS. 63C, 63G, and 63K for 20× and 40× magnifications, respectively). Cell nuclei were counterstained with Hoechst (FIGS. 63B, 63F, and 63J) and cells stained with anti-HA to correlate levels of HA (CasX transgene levels; FIGS. 63D, 63H, and 63L; 40× magnification) and GFP expressed in photoreceptors. White box outlines in B and F indicate retinal regions analyzed at 40× magnification in FIGS. 63C and 63G. Legend: RPE=retinal pigment epithelium, OS=outer segment, ONL=outer nuclear layer, INL=inner nuclear layer, GCL=ganglion.

FIG. 64A shows results of an immunohistochemistry staining of a mouse liver section showing that CasX 491 and gRNA scaffold 174 with spacer 12.7 administered as an AAV IV injection was able to edit the tdTom locus in vivo in Ai9 mice, as described in Example 3. The images are representative of n=3 animals.

FIG. 64B shows results of an immunohistochemistry staining of a mouse heart section showing that CasX 491 and gRNA scaffold 174 with spacer 12.7 administered as an AAV IV injection was able to edit the tdTom locus in vivo in Ai9 mice, as described in Example 3. The images are representative of n=3 animals.

FIG. 65 is a graph of the quantification of percent editing at the exemplary B2M locus 5 days post-transduction of AAVs into human NPCs in a series of three-fold dilution of MOI, as described in Example 17. Editing levels were determined by NGS as indel rate and by flow cytometry as population of cells that do not express the HLA protein due to successful editing at the B2M locus.

FIG. 66 shows the results of an editing assay measured as indel rate detected by NGS at the human AAVS1 locus in human induced neurons (iNs) using the three indicated AAVs, each containing CasX 491 and gRNA with a specific spacer targeting AAVS1, as described in Example 17.

FIG. 67 is a bar graph exhibiting percent editing at the B2M locus in human iNs 14 days post-transduction of AAVs expressing CasX 491 driven by various protein promoters at an MOI of 2E4 or 6.67E3, as described in Example 17.

FIG. 68 shows the results of an editing assay using AAV transgene plasmids nucleofected into hNPCs, as described in Example 18, demonstrating that CpG reduction or depletion within the U1a promoter (construct ID 178 and 179), U6 promoter (construct ID 180 and 181), or bGH poly(A) (construct ID 182) did not significantly reduce CasX-mediated editing at the B2M locus compared to the editing achieved with the original CpG+AAV vector (construct ID 177). The controls used in this experiment were the non-targeting (NT) spacer and no treatment (NTx).

FIG. 69 is a bar graph showing editing results of the tdTomato locus in an experiment to assess the effects of AAV constructs having engineered Pol III promoter hybrid variants when delivered to mNPCs in an AAV vector, as described in Example 18. Editing was assessed by FACS five days post-nucleofection.

FIG. 70 illustrates the quantification of percent editing at the B2M locus as detected by NGS seven days post-transduction of AAVs into human iNs at an MOI of 3E3 (top bar chart) or 1E3 (middle bar chart), as described in Example 18. Various CpG-reduced or CpG-depleted AAV elements were tested (bottom table) to assess the effects of their use on editing efficiency at the B2M locus.

FIG. 71 is a bar plot showing the quantification of percent editing measured as indel rate detected by NGS at the ROSA26 locus for the indicated AAV constructs nucleofected into C2C12 myoblasts or mouse NPCs to assess the effects of individual muscle-specific promoters on editing rates, as described in Example 21.

FIG. 72 is a scatter plot of percent editing versus promoter size for all the AAV constructs with varying promoters tested, as described in Example 21.

FIG. 73 is a bar graph showing editing results of the tdTomato locus in an experiment to assess the effects of AAV constructs having engineered Pol III promoter hybrid variants when delivered to mNPCs in an AAV vector, as described in Example 5. Editing was assessed by FACS five days post-nucleofection.

FIG. 74A is a bar plot showing the quantification of percent editing at the B2M locus in human induced neurons (iNs) transduced with AAVs expressing the indicated constructs containing various poly(A) signal sequences at an MOI of 1E2 vg/cell, as described in Example 6.

FIG. 74B is a bar plot showing the quantification of percent editing at the B2M locus in human induced neurons (iNs) transduced with AAVs expressing the indicated constructs containing various poly(A) signal sequences at an MOI of 1E3 vg/cell, as described in Example 6.

FIG. 75 shows the schematics of AAV constructs with additional alternative gRNA configurations for constructs having two gRNAs, as described in Example 9. The tapered points depict the orientation of the transcriptional unit for CasX protein or gRNA.

FIG. 76A is a diagram of the secondary structure of guide RNA scaffold 235, noting the regions with CpG motifs, as described in Example 18. CpG motifs in (1) the pseudoknot stem, (2) the scaffold stem, (3) the extended stem bubble, (4) the extended step, and (5) the extended stem loop are labeled on the structure.

FIG. 76B is a diagram of the CpG-reducing mutations that were introduced into each of the five regions in the coding sequence of the guide RNA scaffold, as described in Example 18.

FIG. 77A provides the results of an editing experiment in which AAV vectors with various CpG-reduced or CpG-depleted guide RNA scaffolds were used to edit the B2M locus in induced neurons, as described in Example 18. The AAV vectors were administered at a multiplicity of infection (MOI) of 4e3. The bars show the mean±the SD of two replicates per sample. “No Tx” indicates a non-transduced control, and “NT” indicates a control with a non-targeting spacer.

FIG. 77B provides the results of an editing experiment in which AAV vectors with various CpG-reduced or CpG-depleted guide RNA scaffolds were used to edit the B2M locus in induced neurons, as described in Example 18. The AAV vectors were administered at an MOI of 3e3. The bars show the mean±the SD of two replicates per sample. “No Tx” indicates a non-transduced control.

FIG. 77C provides the results of an editing experiment in which AAV vectors with various CpG-reduced or CpG-depleted guide RNA scaffolds were used to edit the B2M locus in induced neurons, as described in Example 18. The AAV vectors were administered at an MOI of 1e3. The bars show the mean±the SD of two replicates per sample. “No Tx” indicates a non-transduced control.

FIG. 77D provides the results of an editing experiment in which AAV vectors with various CpG-reduced or CpG-depleted guide RNA scaffolds were used to edit the B2M locus in induced neurons, as described in Example 18. The AAV vectors were administered at an MOI of MOI=3e2. The bars show the mean±the SD of two replicates per sample. “No Tx” indicates a non-transduced control.

FIG. 78A is a bar graph showing the quantification of percent editing measured as indel rate detected at the ROSA26 locus in C2C12 myoblasts and myotubes transduced with AAVs containing the indicated promoters to drive CasX expression at an MOI of 3E5 vg/cell, as described in Example 21.

FIG. 78B is a bar graph showing the quantification of percent editing measured as indel rate detected at the ROSA26 locus in C2C12 myoblasts and myotubes transduced with AAVs containing the indicated promoters to drive CasX expression at an MOI of 1E5 vg/cell, as described in Example 21.

FIG. 79 is a bar graph showing the quantification of percent editing measured as indel rate detected at the ROSA26 locus in the indicated tissues harvested from mice injected with AAVs containing the indicated promoters driving CasX expression, as described in Example 21. As experimental controls, mice were either untreated (naïve) or injected with AAVs containing UbC promoter driving CasX expression with a non-targeting gRNA. N=3 animals per promoter experimental condition; N=2 animals for the untreated control group.

FIG. 80 is a bar graph quantifying average CasX expression, normalized by vg/dg, driven by muscle-specific promoters CK8e or MHC7 relative to CasX expression driven by UbC, for the indicated tissues harvested from mice injected with AAVs containing the indicated promoters, as described in Example 21. N=3 animals per promoter experimental condition.

FIG. 81 is a box plot showing the quantification of percent editing at the ROSA26 locus in retinae harvested from mice treated with subretinal injections of AAVs expressing CasX 491 driven by the indicated photoreceptor-specific promoters with a ROSA26-targeting spacer, as described in Example 28. The dashed line indicates the theoretical maximum editing of photoreceptors that can be achieved with optimal transduction.

FIG. 82A is a panel of scatterplots for promoter variants GRK1(292)-SV40 and GRK1(292), showing the correlation of vg/dg with the editing level achieved for a particular promoter used to drive CasX expression in the retinae, as described in Example 28. A nonlinear regression curve was fitted to assess the correlation, and the values of the slopes, along with their corresponding standard deviation values, of these curves were determined and reported in Table 47.

FIG. 82B is a panel of scatterplots for promoter variants GRK1(241) and GRK1(199), showing the correlation of vg/dg with the editing level achieved for a particular promoter used to drive CasX expression in the retinae, as described in Example 28. A nonlinear regression curve was fitted to assess the correlation, and the values of the slopes, along with their corresponding standard deviation values, of these curves were determined and reported in Table 47.

FIG. 82C is a panel of scatterplots for the indicated promoter variants GRK1(94) and GRK1(93), showing the correlation of vg/dg with the editing level achieved for a particular promoter used to drive CasX expression in the retinae, as described in Example 28. A nonlinear regression curve was fitted to assess the correlation, and the values of the slopes, along with their corresponding standard deviation values, of these curves were determined and reported in Table 47.

FIG. 83 is a bar plot showing the results of an editing assay at the tdTomato locus assessed by FACS in mNPCs nucleofected with AAV plasmids encoding for XAAVs expressing the CasX:dual-gRNA system with the indicated configurations and spacer combinations for the two gRNA units relative to the CasX construct, as described in Example 29. The “R” preceding the spacer denotes the reverse orientation of the transcription of the indicated gRNA unit. An AAV plasmid encoding for AAVs expressing CasX 491 with a single gRNA transcriptional unit using spacer 12.7 as well as an untreated well served as experimental controls.

FIG. 84A is a line graph showing the results of an editing assay at the tdTomato locus assessed by FACS in mNPCs transduced with XAAVs expressing the CasX:dual-gRNA system at varying MOIs, with the indicated spacer combinations of the two gRNA units arranged in configuration #1 relative to the CasX construct, as described in Example 29. An untreated control was included for comparison.

FIG. 84B is a line graph showing the results of an editing assay at the tdTomato locus assessed by FACS in mNPCs transduced with XAAVs expressing the CasX:dual-gRNA system at varying MOIs, with the indicated spacer combinations of the two gRNA units arranged in configuration #4 relative to the CasX construct, as described in Example 29. The “R” preceding the spacer denotes the reverse orientation of the transcription of the indicated gRNA unit. An untreated control was included for comparison.

FIG. 84C is a line graph showing the results of an editing assay at the tdTomato locus assessed by FACS in mNPCs transduced with XAAVs expressing the CasX:dual-gRNA system at varying MOIs, with the indicated spacer combinations of the two gRNA units arranged in configuration #2 relative to the CasX construct, as described in Example 29 An untreated control was included for comparison.

FIG. 85 is a bar graph showing the results of an editing assay at the tdTomato locus assessed by FACS in mNPCs transduced with XAAVs expressing the CasX:dual-gRNA system for indicated configurations #1, #4, and #2, as described in Example 29. XAAVs expressing CasX 491 with a single gRNA transcriptional unit using spacer 12.7 served as an experimental control.

FIG. 86 is a bar plot showing percent editing at the AAVS1 locus in human induced neurons (iNs) transduced with AAVs expressing the CasX:gRNA system using the indicated U6 promoter variants, at the MOI of 1E3 and 3E2 vg/cell, for N=1, as described in Example 31.

FIG. 87 is a bar plot showing percent editing at the AAVS1 locus in human iNs transduced with AAVs expressing the CasX:gRNA system using the indicated U6 promoter variants, at the MOI of 1E3 and 3E2 vg/cell, for N=2, as described in Example 31.

FIG. 88 is a bar plot showing percent editing at the AAVS1 locus in human iNs transduced with AAVs expressing the CasX:gRNA system using the indicated U6 promoter variants, at the MOI of 2E3, 6.67E2, and 2E2 vg/cell, for N=1, as described in Example 31.

FIG. 89 is a bar plot showing percent editing at the AAVS1 locus in human iNs transduced with AAVs expressing the CasX:gRNA system using the indicated U6 promoter variants, at the MOI of 2E3, 6.67E2, and 2E2 vg/cell, for N=2, as described in Example 31.

FIG. 90 is a bar plot showing percent editing at the AAVS1 locus in human iNs transduced with AAVs expressing the CasX:gRNA system using the indicated U6 promoter variants, at the MOI of 3E4, 1E4, 3.33E3, and 1.11E3 vg/cell, for N=1, as described in Example 31.

FIG. 91 is a bar plot showing percent editing at the AAVS1 locus in human iNs transduced with AAVs expressing the CasX:gRNA system using the indicated CasX proteins, at the MOI of 3E3, 1E3, and 3E2 vg/cell, as described in Example 34.

FIG. 92A is a bar plot showing percent editing at the AAVS1 locus in human iNs transduced with AAVs expressing the CasX:gRNA system using the indicated protein promoter and WPRE elements, at the MOI of 1E3 vg/cell, as described in Example 35.

FIG. 92B is a bar plot showing percent editing at the AAVS1 locus in human iNs transduced with AAVs expressing the CasX:gRNA system using the indicated protein promoter and WPRE elements, at the MOI of 1E4 vg/cell, as described in Example 35.

FIG. 93 is a bar graph showing the quantification of percent knockout of B2M in HEK293 cells transfected with CpG-depleted AAV plasmids containing the indicated gRNA scaffolds with spacer 7.37, as described in Example 33. The dotted line annotates the ˜41% transfection efficiency.

FIG. 94A is a bar plot showing percent editing at the AAVS1 locus in human iNs transduced with AAVs expressing the CasX:gRNA system using the indicated gRNA scaffolds (AAV construct ID #262-274) at the MOI of 3E4 vg/cell, as described in Example 33.

FIG. 94B is a bar plot showing percent editing at the AAVS1 locus in human iNs transduced with AAVs expressing the CasX:gRNA system using the indicated gRNA scaffolds (AAV construct ID #262-274) at the MOI of 1E4 vg/cell, as described in Example 33.

FIG. 94C is a bar plot showing percent editing at the AAVS1 locus in human iNs transduced with AAVs expressing the CasX:gRNA system using the indicated gRNA scaffolds (AAV construct ID #262-274) at the MOI of 3E3 vg/cell, as described in Example 33.

FIG. 95A is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 1E4 vg/cell, as described in Example 33.

FIG. 95B is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 3E3 vg/cell, as described in Example 33.

FIG. 95C is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 1E3 vg/cell, as described in Example 33.

FIG. 96 is a western blot showing the levels of CasX expression (top western blot) in HEK293 cells transfected with AAV plasmids containing a CpG+CasX 515 sequence (lane 1) or CpG⁻ v1 CasX 515 sequence (lanes 2-3), as described in Example 32. Lysate from untransfected HEK293 cells were used as a ‘no plasmid’ control (lane 4). The bottom western blot shows the total protein loading control. Three technical replicates are shown.

FIG. 97 is a bar plot showing the results of AAV titering determined via ddPCR using a primer-probe set specific to either BGH or CasXfor the indicated AAV constructs, as described in Example 30.

FIG. 98 is a bar plot showing percent editing at the AAVS1 locus in human induced neurons (iNs) transduced with AAVs expressing the indicated AAV constructs (either dual-guide or single-guide), at the MOI of 1.3E4, 4.33E3, and 1.44E3 vg/cell, for N=1, as described in Example 30.

FIG. 99 is a bar plot showing percent editing at the B2M locus in human iNs transduced with AAVs expressing the indicated AAV constructs (either dual-guide or single-guide), at the MOI of 1.3E4, 4.33E3, and 1.44E3 vg/cell, for N=1, as described in Example 30.

FIG. 100 is a bar plot showing percent editing at the AAVS1 locus in human iNs transduced with AAVs expressing the indicated AAV constructs (either dual-guide or single-guide), at the MOI of 1E4, 3E3, and 1E3 vg/cell, for N=2, as described in Example 30.

FIG. 101 is a bar plot showing percent editing at the B2M locus in human iNs transduced with AAVs expressing the indicated AAV constructs (either dual-guide or single-guide), at the MOI of 1E4, 3E3, and 1E3 vg/cell, for N=2, as described in Example 30.

FIG. 102 shows the results of an editing experiment in which HEK293T cells were transduced with lentiviral particles expressing CasX variant 515 and a gRNA made up of either gRNA scaffold 174, 235, 316, 382, or 392 targeting the B2M locus or a non-targeting (“NT”) control, as described in Example 39. The lentiviruses were transduced at an MOI of 0.1. The bars show the mean of three samples, and the error bars represent the standard error of the mean (SEM).

FIG. 103 shows the results of an editing experiment in which HEK293T cells were transduced with lentiviral particles expressing CasX variant 515 and a gRNA made up of either gRNA scaffold 174, 235, 316, 382, or 392 targeting the B2M locus or a non-targeting (“NT”) control, as described in Example 39. The lentiviruses were transduced at a MOI of 0.05. The bars show the mean of three samples, and the error bars represent the SEM.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like). Thus, the skilled artisan will understand that while individual bases within a sequence may not be complementary to another sequence, the sequence as a whole is still considered to be complementary.

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include, inter alia, polyadenylation signals (poly(A) signal), enhancer elements, introns, posttranscriptional regulatory elements (PTREs), nuclear localization signals (NLS), deaminases, DNA glycosylase inhibitors, factors that stimulate CRISPR-mediated homology-directed repair (e.g. in cis or in trans), activators or repressors of transcription, self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art. All Pol II promoters are envisaged as within the scope of the instant disclosure.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure. Non-limiting examples of enhancers include CMV enhancer, muscle enhancer, cardiac muscle enhancer, skeletal muscle enhancer, myoblast muscle enhancer, and PTRE.

As used herein, a “post-transcriptional regulatory element (PTRE, or TRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

“Dissociation constant”, or “Ka”, are used interchangeably and mean the affinity between a ligand “L” and a protein “P”; i.e., how tightly a ligand binds to a particular protein. It can be calculated using the formula K_{d}=[L][P]/[LP], where [P], [L] and [LP] represent molar concentrations of the protein, ligand and complex, respectively.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like. Modifying can also encompass epigenetic modifications to a nucleic acid, or chromatin containing the nucleic acid, such as, but not limited to, changes in DNA methylation, and histone methylation and acetylation.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” comprises a replicon, such as plasmid, phage, virus, or cosmid, to which another DNA segment, (e.g., an expression cassette), may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an AAV vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an AAV vector.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., in a cell line), which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for an antibody fragment or glycoprotein tropism factor.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “antibody,” as used herein, encompasses various antibody structures, including but not limited to monoclonal antibodies, polyclonal antibodies, multispecific antibodies (e.g., bispecific antibodies), nanobodies, single domain antibodies such as VHH antibodies, and antibody fragments so long as they exhibit the desired antigen-binding activity or immunological activity. Antibodies represent a large family of molecules that include several types of molecules, such as IgD, IgG, IgA, IgM and IgE.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, a single domain antibody, a single domain camelid antibody, single-chain variable fragment (scFv) antibody molecules, and multispecific antibodies formed from antibody fragments.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

Some of the numerical results herein, for example multiplicity of infection (MOI), are expressed in scientific notation, in which a numerical value is expressed as a number multiplied by 10 raised to a certain exponent. There are various well-known ways to express a number in scientific notation. For example, each of 1E9, 1e9, 1e+9, or 1×10⁹ are variant formats of scientific notation, and is known to have the same meaning of 1 times 10 to the power of 9, or 1,000,000,000.

All publications, patents, and patent applications mentioned in this specification are herein incorporated by reference to the same extent as if each individual publication, patent, or patent application was specifically and individually indicated to be incorporated by reference. The contents of WO 2020/247882, filed on Jun. 5, 2020, WO 2020/247883, filed Jun. 5, 2020, WO 2021/050593, filed on Sep. 9, 2020, WO 2021/050601, filed on Sep. 9, 2021, WO 2021/142342, filed on Jan. 8, 2021, WO 2021/113763, filed on Dec. 4, 2020, WO 2021/113769, filed on Dec. 4, 2020, WO 2021/113772, filed on Dec. 4, 2020, WO 2022/120095, filed Dec. 2, 2021, WO 2022/120094, filed on Dec. 2, 2021, WO 2022/125843, filed on Dec. 9, 2021, WO 2022/261150, filed on Jun. 7, 2022, WO 2023/049742, filed on Sep. 21, 2022, WO 2022/261149, filed on Jun. 7, 2022, PCT/US2023/067791, filed on Jun. 1, 2023, and PCT/US2023/067901, filed on Jun. 3, 2023 which disclose CasX variants and gRNA variants, are hereby incorporated by reference in their entirety.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Recombinant AAV Vectors

In a first aspect, the present disclosure relates to recombinant AAV vectors (rAAV) optimized for the expression and delivery of CRISPR nucleases to target cells and/or tissues for genetic editing.

Wild-type AAV is a small, single-stranded DNA virus belonging to the parvovirus family. The wild-type AAV genome is made up of two genes that encode four replication proteins and three capsid proteins, respectively, and is flanked on either side by inverted terminal repeats (ITRs) having 130-145 nucleotides that fold into a hairpin shape important for replication. The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). The cap gene produces an additional, non-structural protein called the Assembly-Activating Protein (AAP). This protein is produced from ORF2 and is essential for the capsid-assembly process. The capsid forms a supramolecular assembly of approximately 60 individual capsid protein subunits into a non-enveloped, T-1 icosahedral lattice capable of protecting the AAV genome.

Being naturally replication-defective and capable of transducing nearly every cell type in the human body, AAV represents a suitable vector for therapeutic use in gene therapy or vaccine delivery. Typically, when producing a recombinant AAV vector, the sequence between the two ITRs is replaced with one or more sequences of interest as a part of the transgene, and the Rep and Cap sequences are provided in trans, making the ITRs the only viral DNA that remains in the vector. The resulting recombinant AAV vector genome construct comprises two cis-acting 130 to 145-nucleotide ITRs flanking an expression cassette encoding the transgene sequences of interest, providing at least 4.7 kb or more for packaging of foreign DNA such that the total size of the vector is below 4.8 to 5 kb, which is compatible with packaging within the AAV capsid (it being understood that as the size of the construct exceeds this threshold, the packaging efficiency of the vector decreases). As used herein, “transgene” includes ITRs and an expression cassette incorporated between the ITRs. In the context of CRISPR-mediated gene editing, however, the size limitation of the expression cassette is a challenge for most CRISPR systems for incorporation into an AAV, given the large size of the nucleases.

In one aspect, the present disclosure relates to rAAV transgene compositions. In some embodiments, the disclosure provides transgenes wherein the transgene comprises a polynucleotide sequence encoding a Class 2, Type V CRISPR nuclease protein and a polynucleotide sequence encoding a first guide RNA (gRNA) with a linked targeting sequence of 15 to 20 nucleotides complementary to a target nucleic acid of a cell. In some embodiments, the disclosure provides an rAAV transgene comprising a polynucleotide sequence encoding a CasX nuclease protein and a polynucleotide sequence encoding a first guide RNA (gRNA) with a linked targeting sequence of 15 to 20 nucleotides complementary to a target nucleic acid of a cell. In some embodiments, the disclosure provides an rAAV transgene comprising a polynucleotide sequence encoding a CasX nuclease protein, and a polynucleotide sequence encoding a first and a second guide RNA (gRNA), each with a linked targeting sequence of 15 to 20 nucleotides complementary to a target nucleic acid of a cell, wherein the targeting sequence of the second gRNA is complementary to a different or overlapping region of the target nucleic acid. In some embodiments, the transgene has less than about 4800, less than about 4700, less than about 4600, less than about 4500, less than about 4400 nucleotides, less than about 4300 nucleotides, or less than about 4250 nucleotides, and the rAAV transgene is configured for incorporation into an rAAV capsid. In some embodiments, the transgene has about 4250 to about 4800 nucleotides, or any integer in between. The CasX nuclease, gRNA, and other components of the rAAV transgene are described more fully, below.

In some embodiments, the transgene comprises components selected from a polynucleotide sequence encoding a CasX nuclease protein and a polynucleotide sequence encoding a first guide RNA (gRNA) with a linked targeting sequence of 15 to 20 nucleotides complementary to a target nucleic acid of a cell, a first and a second rAAV inverted terminal repeat (ITR) sequence, a first promoter sequence operably linked to the CasX protein, a sequence encoding a nuclear localization signal (NLS), a 3′ UTR, a poly(A) signal sequence, a second promoter operably linked to the first gRNA, and, optionally, an accessory element, wherein the rAAV transgene is configured for incorporation into an rAAV capsid. In some embodiments, the transgene comprises components selected from a polynucleotide sequence encoding a CasX nuclease protein and a polynucleotide sequence encoding a first guide RNA (gRNA) with a linked targeting sequence of 15 to 20 nucleotides complementary to a target nucleic acid of a cell, a first and a second rAAV inverted terminal repeat (ITR) sequence, a first promoter sequence operably linked to the CasX protein, a sequence encoding a nuclear localization signal (NLS), a 3′ UTR, a poly(A) signal sequence, a second promoter operably linked to the first gRNA, a second gRNA, a third promoter operably linked to the second gRNA, and, optionally, an accessory element, wherein the rAAV transgene is configured for incorporation into an rAAV capsid.

The promoter and accessory elements can be operably linked to components within the transgene, e.g., the CRISPR protein and/or gRNA, in a manner which permits its transcription, translation and/or expression in a cell transfected with the rAAV of the embodiments. As used herein, “operably linked” sequences include both accessory element sequences that are contiguous with the gene of interest and accessory element sequences that are at a distance to control the gene of interest.

In some embodiments, the disclosure provides accessory elements for inclusion in the rAAV that include, but are not limited to sequences that control transcription initiation, termination, enhancer elements, RNA processing signal sequences, enhancer elements, sequences that stabilize cytoplasmic mRNA, sequences that enhance translation efficiency (i.e., Kozak consensus sequence), an intron, a post-transcriptional regulatory element (PTRE), a deaminase, a DNA glycosylase inhibitor, a stimulator of CRISPR-mediated homology-directed repair, and an activator or repressor of transcription. In some cases, the PTRE is selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70). In some embodiments, the PTRE comprises a sequence selected from the group consisting of SEQ ID NOS: 3615-3617, or a sequence having at least about 70%, at least about 80%, at least about 85%, at least about 90%, at least about 95%, at least about 95%, at least about 96%, at least about 97%, at least about 98%, or at least about 99% identity thereto. In the foregoing, the one or more accessory elements are operably linked to the CRISPR protein. It has been discovered that the inclusion of the accessory element(s) in the polynucleotide of the rAAV construct can enhance the expression, binding, activity, or performance of the CRISPR protein as compared to the CRISPR protein in the absence of said accessory element in the transgene of an rAAV vector. In one embodiment, the inclusion of the one or more accessory elements the transgene of the rAAV results in an increase in editing of a target nucleic acid by the CRISPR protein in an in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% as compared to the CRISPR protein in the absence of said accessory element in an rAAV vector.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV11, AAV12, AAV 9.45, AAV 9.61, AAV 44.9, AAV-Rh74, AAVRh10, MyoAAV 1A1, MyoAAV 1A2, and MyoAAV 2A, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein). In some embodiments, the ITRs are derived from serotype AAV1. In other embodiments, the ITRs are derived from serotype AAV2, including the 5′ ITR having sequence CCTGCAGGCAGCTGCGCGCTCGCTCGCTCACTGAGGCCGCCCGGGCGTCGGGCGAC CTTTGGTCGCCCGGCCTCAGTGAGCGAGCGAGCGCGCAGAGAGGGAGTGGCCAACT CCATCACTAGGGGTTCCT (SEQ ID NO: 17) and the 3′ ITR having sequence AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGCTGCCTGCAGG (SEQ ID NO: 18). In other embodiments, the ITR sequences are modified to remove CpG motifs to reduce immunogenic responses. In one embodiment, the modified AAV2 5′ ITR sequence is the sequence of SEQ ID NO: 3749 and the 3′ ITR sequence is the sequence of SEQ ID NO: 4047.

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome.

By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, the rAAV is of serotype 9 or of serotype 6, which have been demonstrated to effectively deliver polynucleotides to motor neurons and glia throughout the spinal cord in preclinical models of Amyotrophic lateral sclerosis (ALS) (Foust, K D. et al. Therapeutic AAV9-mediated suppression of mutant RHO slows disease progression and extends survival in models of inherited ALS. Mol Ther. 21(12):2148 (2013)). In some embodiments, the methods provide use of rAAV9 or rAAV6 for targeting of neurons via intraparenchymal brain injection. In some embodiments, the methods provide use of rAAV9 for intravenous administering of the vector wherein the rAAV9 has the ability to penetrate the blood-brain barrier and drive gene expression in the nervous system via both neuronal and glial tropism of the vector. In other embodiments, the rAAV is of serotype 8, which have been demonstrated to effectively deliver polynucleotides to retinal cells.

In a feature of the rAAV of the present disclosure, it has been discovered that utilization of certain Class 2 CRISPR systems of smaller size permit the inclusion of additional sequence space in the polynucleotides used in the making of the rAAV that can be utilized for the remaining components of the transgene, as described herein. In some embodiments, the encoded Class 2 CRISPR system comprises a Type V protein selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ, and the associated guide RNA of the respective system. In some embodiments, the encoded Class 2, Type V CRISPR nuclease protein is a CasX protein. In some embodiments, the encoded Class 2, Type V CRISPR nuclease protein is a CasX, and the guide is a CasX guide; embodiments of which are described herein.

As described, supra, the smaller size of the Class 2, Type V proteins and gRNA contemplated for inclusion in the transgene of the rAAV permit inclusion of additional or larger components in a transgene that can be incorporated into a single rAAV particle. In some embodiments, the transgene encoding the Class 2, Type V proteins and a first gRNA with a linked targeting sequence complementary to a target nucleic acid and one or more accessory elements has less than about 4800, less than about 4700, less than about 4600, less than about 4500, less than about 4400 nucleotides, less than about 4300 nucleotides, or less than about 4250 nucleotides, wherein the rAAV transgene is configured for incorporation into a rAAV capsid. In other embodiments, the transgene encoding the Class 2, Type V proteins and a first and a second gRNA with linked targeting sequences complementary to a target nucleic acid and one or more accessory elements has less than about 4800, less than about 4700, less than about 4600, less than about 4500, less than about 4400 nucleotides, less than about 4300 nucleotides, or less than about 4250 nucleotides, wherein the rAAV transgene is configured for incorporation into a rAAV capsid. In some embodiments, the rAAV transgene has about 4250 to about 4800 nucleotides, or any integer in between.

In some embodiments, the polynucleotide of the transgene encoding the Class 2, Type V CRISPR nuclease protein sequence and the gRNA sequence are less than about 3100, about 3090, about 3080, about 3070, about 3060, about 3050, or less than about 3040 nucleotides in length. In other embodiments, the polynucleotide of the transgene encoding the Class 2, Type V CRISPR nuclease protein sequence and the gRNA sequence are less than about 3040 to about 3100 nucleotides in combined length. Thus, in light of the total length of the expression cassette that can be packaged into an rAAV particle, in some embodiments, the polynucleotide sequences of the transgene of a first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the transgene of the first promoter and the at least one accessory element have greater than at least about 1300 to at least about 1900 nucleotides in combined length. In one embodiment, the polynucleotide sequences of the transgene of the first promoter and the at least one accessory element have greater than 1314 nucleotides in combined length. In another embodiment, the polynucleotide sequences of the transgene of the first promoter and the at least one accessory element have greater than 1381 nucleotides in combined length. In other embodiments, the polynucleotide sequences of the transgene of the first promoter, the second promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the transgene of the first promoter, the second promoter and the at least one accessory element have greater than at least about 1300 to at least about 1900 nucleotides in combined length. In one embodiment, the polynucleotide sequences of the transgene of the first promoter, the second promoter, and the at least one accessory element have greater than 1314 nucleotides in combined length. In other embodiments, the polynucleotide sequences of the transgene of the first promoter, the second promoter, and the at least one accessory element have greater than 1381 nucleotides in combined length. In still other embodiments, the polynucleotide sequences of the transgene of the first promoter, the second promoter, and the two or more accessory elements have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the transgene of the first promoter, the second promoter, and the two or more accessory elements have greater than at least about 1300 to at least about 1900 nucleotides in combined length. In one embodiment, the polynucleotide sequences of the transgene of the first promoter, the second promoter, and the two or more accessory elements have greater than 1314 nucleotides in combined length. In another embodiment, the polynucleotide sequences of the transgene of the first promoter, the second promoter, and the two or more accessory elements have greater than 1381 nucleotides in combined length.

It has been discovered that use of shorter or truncated promoters in the rAAV transgene also permits a shorter total transgene size for inclusion of all the CRISPR and regulatory elements, while increasing the percentage of correctly packaged rAAV particles. In some embodiments, the total length of the transgene polynucleotide sequences of the first promoter and at least one accessory element are greater than at least about 1200, at least about 1300, at least about 1350, at least about 1360, at least about 1370, at least about 1380, at least about 1390, at least about 1400, at least about 1500, at least about 1600 nucleotides, at least 1650, at least about 1700 nucleotides in an rAAV construct with a total length of not more than 4700 nucleotides, wherein the transgene is capable of being integrated into an rAAV particle. In other embodiments, the total length of the transgene the polynucleotide sequences of the first promoter and at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700 nucleotides in an rAAV construct with a total length of not more than 4800 nucleotides, wherein the transgene is configured for incorporation into an rAAV particle.

In some embodiments, the present disclosure provides a transgene polynucleotide comprising a first adeno-associated virus (AAV) inverted terminal repeat (ITR) sequence, a second AAV ITR sequence, a first promoter sequence, a sequence encoding a Class 2, Type V CRISPR nuclease protein, a second promoter sequence, a sequence encoding at least a first guide RNA (gRNA), and one or more accessory element sequences, wherein at least 25%, 26%, 27%, 28%, 29%, 30%, 31%, 32%, 33%, 34%, or 35% or more of the nucleotides of the polynucleotide sequence comprise the first and second promoters and the one or more accessory element sequences in combined length. In other embodiments, the present disclosure provides a transgene polynucleotide comprising a first adeno-associated virus (AAV) inverted terminal repeat (ITR) sequence, a second AAV ITR sequence, a first promoter sequence, a sequence encoding a Class 2, Type V CRISPR nuclease protein, a second promoter sequence, a sequence encoding a first guide RNA (gRNA), a third promoter sequence, a sequence encoding a second gRNA, and one or more accessory element sequences, wherein at least 25%, 26%, 27%, 28%, 29%, 30%, 31%, 32%, 33%, 34%, or 35% or more of the nucleotides of the polynucleotide sequences comprising the first, second, and third promoters and the one or more accessory element sequences in combined length. As detailed in the Examples, it has been discovered that the ability to devote more of the total polynucleotide of the expression cassette of an rAAV transgene to the promoters, a second gRNA, and/or the accessory elements results in enhanced expression of and/or performance of the CRISPR protein and gRNA, when expressed in the target host cell; either in an in vitro assay or in vivo in a subject. In some embodiments, the use of alternative or longer promoters and/or accessory elements (e.g., poly(A) signal, a gene enhancer element, an intron, a posttranscriptional regulatory element (PTRE), a nuclear localization signal (NLS), a deaminase, a DNA glycosylase inhibitor, a stimulator of CRISPR-mediated homology-directed repair, and an activator or repressor of transcription) in the rAAV polynucleotides and resulting rAAV results in an increase in editing of a target nucleic acid of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% when the rAAV is assessed in an in vitro assay compared to a construct not having the alternative or longer promoters and/or accessory elements. In one embodiment, a Pol II promoter sequence of the transgene polynucleotide has at least about 35, at least about 50, at least about 80, at least about 100, at least about 200, at least about 300, at least about 400, at least about 500, at least about 600, at least about 700, or at least about 800 nucleotides. In another embodiments, a Pol III promoter sequence of the transgene polynucleotide has at least about 50, at least about 80, at least about 100, at least about 200, at least about 300, at least about 400, at least about 500, at least about 600, at least about 700, or at least about 800 nucleotides. Embodiments of the promoters are described more fully, below.

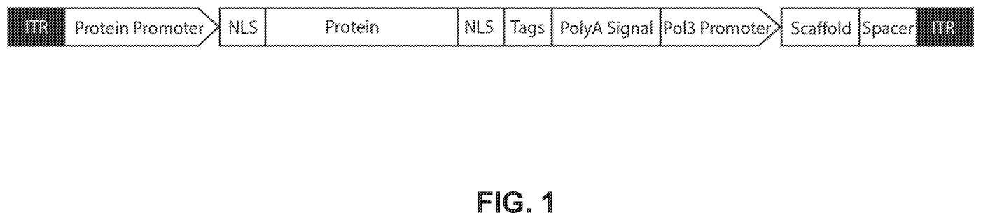

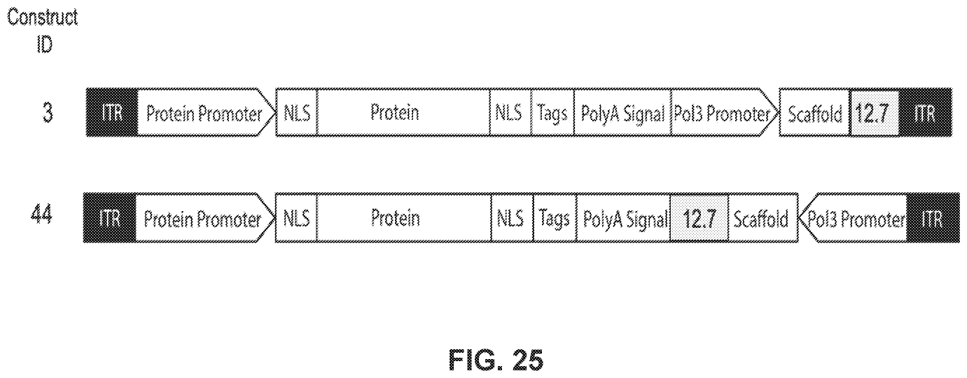

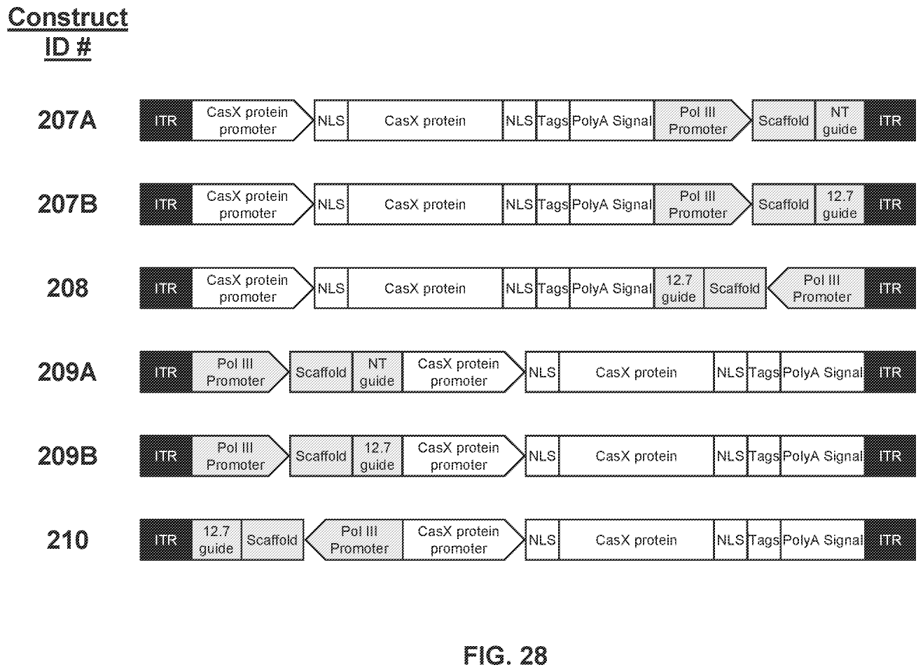

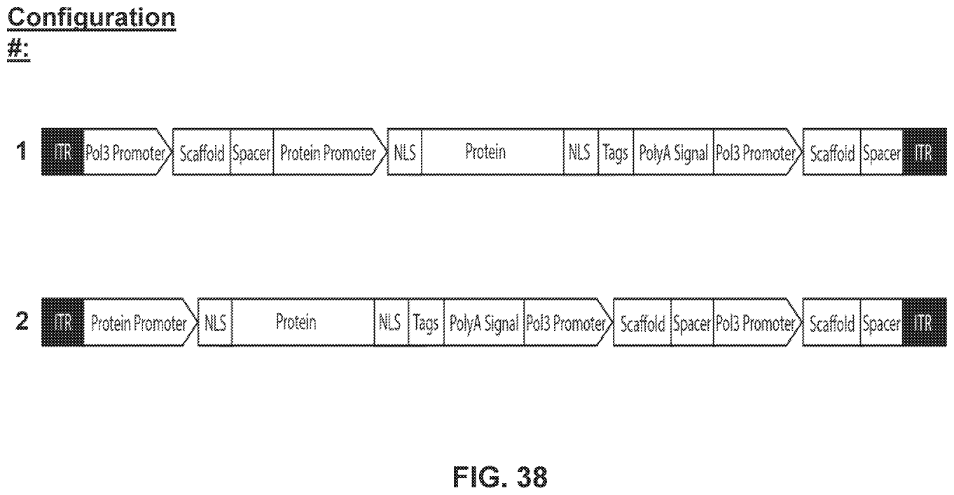

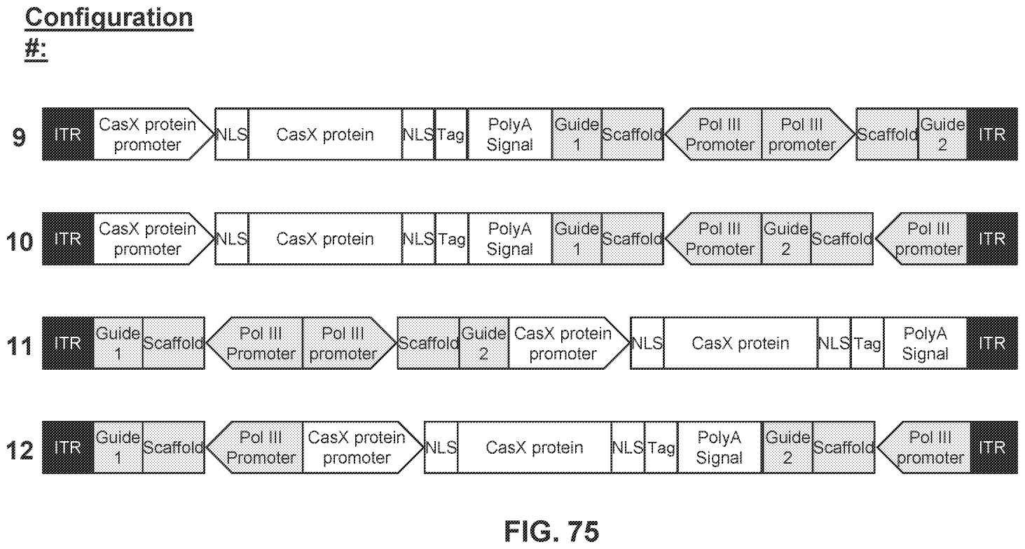

In some embodiments, the present disclosure provides a transgene polynucleotide, wherein the polynucleotide comprises one or more sequences selected from the group of sequences set forth in Tables 7-10, 12-17, 19, 22-38, 39-43, 45-46, 50-55, 57-58, 60-61, and 78 or a sequence having at least 85%, at least 90%, at least 95%, at least 95%, at least 96%, at least 97%, at least 98%, or at least 99% identity thereto. In another embodiment, the present disclosure provides a polynucleotide, wherein the polynucleotide comprises a sequence selected from the group of sequences set forth in Tables 7-10, 12-17, 19, 22-38, 39-43, 45-46, 50-55, 57-58, 60-61, and 78. In some embodiments, the polynucleotide sequence differs from those set forth in Tables 7-10, 12-17, 19, 22-38, 39-43, 45-46, 50-55, 57-58, 60-61, and 78 only in the selection of the targeting sequences of the gRNA or gRNAs encoded by the polynucleotide, wherein the targeting sequence is a sequence having 15 to 20 nucleotides capable of hybridizing with the sequence of a target nucleic acid. In some embodiments, the present disclosure provides a transgene polynucleotide of any of the embodiments described herein, wherein the polynucleotide has the configuration of a construct of FIG. 1, FIG. 25, FIG. 28, FIGS. 38-40, FIG. 47, or FIG. 75.

III. Guide Nucleic Acids of the rAAV

In some embodiments, the disclosure relates to guide ribonucleic acids (gRNA) utilized in the rAAV that have utility in genome editing of a target nucleic acid in a cell. As used herein, the term “gRNA” covers naturally-occurring molecules and gRNA variants, including chimeric gRNA variants comprising domains from different gRNA. gRNAs of the disclosure comprise a scaffold and a targeting sequence complementary to a target nucleic acid of a cell.

The present disclosure provides gRNAs with targeting sequences that are complementary to (and are therefore able to hybridize with) the target nucleic acid as a component of the gene editing rAAV. It is envisioned that in some embodiments, multiple gRNAs are delivered in the rAAV for the modification of a target nucleic acid. For example, a pair of gRNAs with targeting sequences to different or overlapping regions of the target nucleic acid sequence can be used, when each is complexed with a CRISPR nuclease, in order to bind and cleave at two different or overlapping sites within the gene, which is then edited by non-homologous end joining (NHEJ), homology-directed repair (HDR), homology-independent targeted integration (HITI), micro-homology mediated end joining (MMEJ), single strand annealing (SSA) or base excision repair (BER). For example, when an editing event designed to delete one or more exons of a gene is desired, a pair of gRNAs can be used in order to bind and cleave at two different sites 5′ and 3′ of the targeted exon(s) within the gene in order to excise the intervening sequence. In other cases, a pair of gRNAs can be used in order to bind, cleave, and modify two different genes. Both single-stranded cleavage and double-stranded cleavage are possible, and double-stranded cleavage can occur as a result of two distinct single-stranded cleavage events by the CRISPR nuclease.

a. Reference gRNA and gRNA Variants

As used herein, a “reference gRNA” refers to a CRISPR guide ribonucleic acid comprising a wild-type sequence of a naturally-occurring gRNA. In some embodiments, a gRNA scaffold of the disclosure may be subjected to one or more mutagenesis methods, such as the mutagenesis methods described in WO2022120095A1 and WO2020247882A1, incorporated by reference herein, which may include Deep Mutational Evolution (DME), deep mutational scanning (DMS), error prone PCR, cassette mutagenesis, random mutagenesis, staggered extension PCR, gene shuffling, domain swapping, or chemical modification to generate one or more gRNA variants with enhanced or varied properties relative to the gRNA scaffold that was modified. The activity of the gRNA scaffold from which a gRNA variant was derived may be used as a benchmark against which the activity of the gRNA variant is compared, thereby measuring improvements in function or other characteristics of the gRNA scaffold.

Table 1 provides the sequences of reference gRNAs tracr and scaffold sequences. In some embodiments, the disclosure provides gRNA variant sequences wherein the gRNA has a scaffold comprising a sequence having one or more nucleotide modifications relative to a reference gRNA sequence having a sequence of any one of SEQ ID NOS:4-16 of Table 1.

TABLE 1
Reference gRNA tracr and scaffold sequences

| SEQ ID NO. | Nucleotide Sequence |
|---|---|
| 4 | ACAUCUGGCGCGUUUAUUCCAUUACUUUGGAGCCAGUCCCAGCGACUAUGUCGUAUGGACGAAG |
| | CGCUUAUUUAUCGGAGAGAAACCGAUAAGUAAAACGCAUCAAAG |
| 5 | UACUGGCGCUUUUAUCUCAUUACUUUGAGAGCCAUCACCAGCGACUAUGUCGUAUGGGUAAAGC |
| | GCUUAUUUAUCGGAGAGAAAUCCGAUAAAUAAGAAGCAUCAAAG |
| 6 | ACAUCUGGCGCGUUUAUUCCAUUACUUUGGAGCCAGUCCCAGCGACUAUGUCGUAUGGACGAAG |
| | CGCUUAUUUAUCGGAGA |
| 7 | ACAUCUGGCGCGUUUAUUCCAUUACUUUGGAGCCAGUCCCAGCGACUAUGUCGUAUGGACGAAG |
| | CGCUUAUUUAUCGG |
| 8 | UACUGGCGCUUUUAUCUCAUUACUUUGAGAGCCAUCACCAGCGACUAUGUCGUAUGGGUAAAGC |
| | GCUUAUUUAUCGGAGA |
| 9 | UACUGGCGCUUUUAUCUCAUUACUUUGAGAGCCAUCACCAGCGACUAUGUCGUAUGGGUAAAGC |
| | GCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAUGUCGUAUGGGUAAAGCGCUU |
| | AUUUAUCGGA |

b. GRNA Domains and their Function

The gRNAs of the rAAV of the disclosure comprise two segments: a targeting sequence and a protein-binding segment. The targeting segment of a gRNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a strand of a double stranded target DNA, a target ssRNA, a target ssDNA, etc.), described more fully below. The targeting sequence of a gRNA is capable of binding to a target nucleic acid sequence, including, in the context of the present disclosure, a coding sequence, a complement of a coding sequence, a non-coding sequence, and to accessory elements. The protein-binding segment (or “activator” or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below. The properties and characteristics of CasX gRNA, both wild-type and variants, are described in WO2020247882A1, US20220220508A1, and WO2022120095A1, incorporated by reference herein.

In the case of a reference gRNA, the gRNA occurs naturally as a dual guide RNA (dgRNA), wherein the targeter and the activator portions each have a duplex-forming segment that have complementarity with one another and hybridize to one another to form a double stranded duplex (dsRNA duplex for a gRNA). The term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. In the case of the gRNA for use in the systems of the disclosure, the scaffolds are designed such that the activator and targeter portions are covalently linked to one another (rather than hybridizing to one another) and comprise a single molecule, and can be referred to as a “single-molecule gRNA,” “single guide RNA”, a “single-molecule guide RNA,” a “one-molecule guide RNA”, or a “sgRNA”. In some embodiments, the gRNA utilized in the rAAV are single molecule versions.

Collectively, the assembled gRNAs of the disclosure comprise distinct structured regions, or domains: the RNA triplex, the scaffold stem loop, the extended stem loop, the pseudoknot, and the targeting sequence that, in the embodiments of the disclosure is specific for a target nucleic acid and is located on the 3′ end of the gRNA. The RNA triplex, the scaffold stem loop, the pseudoknot and the extended stem loop, together with the unstructured triplex loop that bridges portions of the triplex, together, are referred to as the “scaffold” of the gRNA. In some cases, the scaffold stem further comprises a bubble. In other cases, the scaffold further comprises a triplex loop region. In still other cases, the scaffold further comprises a 5′ unstructured region. In some embodiments, the gRNA scaffolds of the disclosure for use in the CasX:gRNA systems comprise a scaffold stem loop having the sequence of CCAGCGACUAUGUCGUAGUGG (SEQ ID NO: 14), or a sequence having 1, 2, 3, 4, or 5 mismatches thereto.

Each of the structured domains contribute to establishing the global RNA fold of the guide and retain functionality of the guide; particularly the ability to properly complex with the CasX protein. For example, the guide scaffold stem interacts with the helical I domain of CasX protein, while residues within the triplex, triplex loop, and pseudoknot stem interact with the OBD of the CasX protein. Together, these interactions confer the ability of the guide to bind and form an RNP with the CasX that retains stability, while the spacer (or targeting sequence) directs and defines the specificity of the RNP for binding a specific sequence of DNA.

Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC protospacer adjacent motif (PAM) motif or a PAM sequence, such as ATC, CTC, GTC, or TTC. Because the targeting sequence of a guide sequence hybridizes with a sequence of a target nucleic acid sequence, a targeting sequence can be modified by a user to hybridize with a specific target nucleic acid sequence, so long as the location of the PAM sequence is considered. In some embodiments, the target nucleic acid comprises a PAM sequence located 5′ of the targeting sequence with at least a single nucleotide separating the PAM from the first nucleotide of the targeting sequence. In some embodiments, the PAM is located on the non-targeted strand of the target region, i.e. the strand that is complementary to the target nucleic acid. By selection of the targeting sequences of the gRNA, defined regions of the target nucleic acid sequence or sequences bracketing a particular location within the target nucleic acid can be modified or edited using the gRNA and CRISPR nuclease proteins described herein. In some embodiments, the targeting sequence of the gRNA has between 15 and 20 consecutive nucleotides. In some embodiments, the targeting sequence has 15, 16, 17, 18, 19, and 20 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the gRNA and linked targeting sequence exhibit a low degree of off-target effects to the DNA of a cell. As used herein, “off-target effects” refers to effects of unintended cleavage, such as mutations and indel formation, at untargeted genomic sites showing a similar but not an identical sequence compared to the target site (i.e., the sequence complementary to the targeting sequence of the gRNA). In some embodiments, the off-target effects exhibited by the gRNA and linked targeting sequence are less than about 5%, less than about 4%, less than 3%, less than about 2%, less than about 1%, less than about 0.5%, less than 0.1% in cells. In some embodiments, the off-target effects are determined in silico. In some embodiments, the off-target effects are determined in an in vitro cell-free assay. In some embodiments, the off-target effects are determined in a cell-based assay.

In another aspect, the disclosure relates to gRNA variants for use in the rAAV systems, which comprise one or more modifications relative to a reference gRNA scaffold or to another gRNA variant from which it was derived. All gRNA variants that have one or more improved functions, characteristics, or add one or more new functions when the gRNA variant is compared to a reference gRNA or to another gRNA variant from which it was derived, while retaining the functional properties of being able to complex with the CasX and guide the CasX ribonucleoprotein holo complex to the target nucleic acid are envisaged as within the scope of the disclosure. In some embodiments, the gRNA variant has an improved characteristic selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, or any combination thereof. In some cases of the foregoing, the improved characteristic is assessed in an in vitro assay, including the assays of the Examples. In other cases of the foregoing, the improved characteristic is assessed in vivo.

In some embodiments, a reference gRNA of the disclosure may be subjected to one or more mutagenesis methods, such as the mutagenesis methods described herein (as well as in PCT/US20/36506 and WO2020247883A2, incorporated by reference herein), which may include Deep Mutational Evolution (DME), deep mutational scanning (DMS), error prone PCR, cassette mutagenesis, random mutagenesis, staggered extension PCR, gene shuffling, or domain swapping, in order to generate one or more guide nucleic acid variants (referred to herein as “gRNA variant”) with enhanced or varied properties relative to the reference gRNA. gRNA variants also include variants comprising one or more exogenous sequences, for example fused to either the 5′ or 3′ end, or inserted internally. The activity of reference gRNAs may be used as a benchmark against which the activity of gRNA variants are compared, thereby measuring improvements in function or other characteristics of the gRNA variants. In other embodiments, a reference gRNA may be subjected to one or more deliberate, specifically-targeted mutations in order to produce a gRNA variant, for example a rationally designed variant. Exemplary gRNA variants produced by such methods are described in the Examples and representative sequences of gRNA scaffolds are presented in Table 2.

In some embodiments, a gRNA variant for use in the rAAV systems of the disclosure comprises one or more nucleotide substitutions, insertions, deletions, or swapped or replaced regions relative to a reference gRNA sequence of the disclosure that improve a characteristic relative to the reference gRNA. A representative example of such a gRNA variant is guide 235 (SEQ ID NO: 2292). Exemplary regions for modifications include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gRNA variant scaffold comprises a scaffold stem loop having the sequence of CCAGCGACUAUGUCGUAGUGG (SEQ ID NO:14). In another embodiment, the disclosure provides a gRNA scaffold comprising, relative to SEQ ID NO:5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G64U. In the foregoing embodiment, the gRNA scaffold comprises the sequence

(SEQ ID NO: 2238)
ACUGGCGCUUUUAUCUGAUUACUUUGAGAGCCAUCACCAGCGACUAUGU
CGUAGUGGGUAAAGCUCCCUCUUCGGAGGGAGCAUCAAAG.

In exemplary embodiments, a gRNA variant for use in the rAAV systems comprises one or more modifications relative to gRNA scaffold variant 215 (SEQ ID NO:2275), wherein the resulting gRNA variant exhibits an improved functional characteristic compared to the parent 215, when assessed in an in vitro or in vivo assay under comparable conditions.

In exemplary embodiments, a gRNA variant for use in the rAAV systems comprises one or more modifications relative to gRNA scaffold variant 221 (SEQ ID NO: 2281), wherein the resulting gRNA variant exhibits an improved functional characteristic compared to the parent 221, when assessed in an in vitro or in vivo assay under comparable conditions.

In exemplary embodiments, a gRNA variant for use in the rAAV systems comprises one or more modifications relative to gRNA scaffold variant 225 (SEQ ID NO: 2285), wherein the resulting gRNA variant exhibits an improved functional characteristic compared to the parent 225, when assessed in an in vitro or in vivo assay under comparable conditions.

In exemplary embodiments, a gRNA variant for use in the rAAV systems comprises one or more modifications relative to gRNA scaffold variant 235 (SEQ ID NO: 2292), wherein the resulting gRNA variant exhibits an improved functional characteristic compared to the parent 225, when assessed in an in vitro or in vivo assay under comparable conditions.

In exemplary embodiments, a gRNA variant for use in the rAAV systems comprises one or more modifications relative to gRNA scaffold variant 251 (SEQ ID NO: 2308), wherein the resulting gRNA variant exhibits an improved functional characteristic compared to the parent 251, when assessed in an in vitro or in vivo assay under comparable conditions.

In exemplary embodiments, a gRNA variant for use in the rAAV systems comprises one or more modifications relative to gRNA scaffold 316 (SEQ ID NO: 9588), wherein the resulting gRNA variant exhibits an improved functional characteristic compared to the parent 235, when assessed in an in vitro or in vivo assay under comparable conditions.

Table 2 provides exemplary gRNA scaffold sequences of the disclosure for use in the rAAV. In some embodiments, the rAAV comprises a first and a second gRNA, wherein the first and/or the second gRNA are identical. In other embodiments, the rAAV comprises a first and a second gRNA, wherein the first and/or the second gRNA are different. In both cases, the first and the second gRNA would comprise targeting sequences complementary to different target nucleic acid sequences. In some embodiments, the encoded gRNA scaffold for use in the rAAV comprises a sequence selected from the group consisting of SEQ ID NOS: 2238-2400, 9257-9289 and 9588, (of which 2238-2285, 2287-2352, 2376, 2378, 2383-2400, and 9588 are presented in Table 2), wherein the gRNA variant retains the ability to form an RNP with a CasX and to bind a target nucleic acid. In other embodiments, the encoded gRNA variant scaffold for use in the rAAV of the disclosure comprises a sequence having at least about 70%, at least about 80%, at least about 90%, at least about 95%, at least about 95%, at least about 96%, at least about 97%, at least about 98%, at least about 99% sequence identity to a sequence selected from the group consisting of SEQ ID NOS: 2238-2400, 9257-9289 and 9588, wherein the gRNA variant retains the ability to form an RNP with a CasX of the disclosure and to bind a target nucleic acid. In other embodiments, the encoded gRNA variant scaffold for use in the rAAV of the disclosure comprises a sequence selected from the group consisting of SEQ ID NOS: 2238-2400, 9257-9289 and 9588, further comprising 1, 2, 3, 4, or 5 mismatches thereto, wherein the gRNA variant retains the ability to form an RNP with a CasX of the disclosure and to bind a target nucleic acid, whereupon the RNP modifies the target nucleic acid. In one embodiment, the encoded gRNA variant scaffold for use in the rAAV of the disclosure comprises a sequence of SEQ ID NO: 2292. In another embodiment, the encoded gRNA variant scaffold for use in the rAAV of the disclosure comprises a sequence of SEQ ID NO: 9588. It will be understood that in those embodiments wherein the rAAV transgene comprises a DNA encoding sequence for a gRNA, that thymine (T) bases can be substituted for the uracil (U) bases of any of the gRNA sequence embodiments described herein.

TABLE 2
Exemplary gRNA Scaffold Sequences

| SEQ ID | | |
|---|---|---|
| NO: | Name | NUCLEOTIDE SEQUENCE OR DESCRIPTION OF MODIFICATION |
| 2238 | 174 | ACUGGCGCUUUUAUCUGAUUACUUUGAGAGCCAUCACCAGCGACUAUGUCGUAGUGGGU |
| | | AAAGCUCCCUCUUCGGAGGGAGCAUCAAAG |
| 2239 | 175 | ACUGGCGCCUUUAUCUCAUUACUUUGAGAGCCAUCACCAGCGACUAUGUCGUAUGGGUA |
| | | AAGCGCUUACGGACUUCGGUCCGUAAGAAGCAUCAAAG |
| 2240 | 176 | GCUGGCGCUUUUAUCUGAUUACUUUGAGAGCCAUCACCAGCGACUAUGUCGUAGUGGGU |
| | | AAAGCUCCCUCUUCGGAGGGAGCAUCAAAG |
| 2241 | 177 | ACUGGCGCUUUUAUCUGAUUACUUUGAGAGCCAUCACCAGCGACUAUGUCGUAUGGGUA |
| | | AAGCUCCCUCUUCGGAGGGAGCAUCAAAG |
| 2242 | 181 | ACUGGCGCCUUUAUCUGAUUACUUUGAGAGCCAUCACCAGCGACUAUGUCGUAUGGGUA |
| | | AAGCGCUUACGGACUUCGGUCCGUAAGAAGCAUCAAAG |
| 2243 | 182 | ACUGGCGCUUUUAUCUGAUUACUUUGAGAGCCAUCACCAGCGACUAUGUCGUAUGGGUA |
| | | AAGCGCUUACGGACUUCGGUCCGUAAGAAGCAUCAAAG |
| 2244 | 183 | ACUGGCGCUUUUAUCUGAUUACUUUGAGAGCCAUCACCAGCGACUAUGUCGUAGUGGGU |
| | | AAAGCGCUUACGGACUUCGGUCCGUAAGAAGCAUCAAAG |
| 2245 | 184 | ACUGGCGCUUUUAUCUGAUUACUUUGAGAGCCAUCACCAGCGACUAUGUCGUAUUGGGU |
| | | AAAGCUCCCUCUUCGGAGGGAGCAUCAAAG |
| 2246 | 185 | ACUGGCGCUUUUAUCUGAUUACUUUGAGAGCCAUCACCAGCGACUAUGUCGUAUUGGGU |
| | | AAAGCGCUUACGGACUUCGGUCCGUAAGAAGCAUCAAAG |
| 2247 | 186 | ACUGGCGCCUUUAUCAUCAUUACUUUGAGAGCCAUCACCAGCGACUAUGUCGUAUGGGU |
| | | AAAGCGCUUACGGACUUCGGUCCGUAAGAAGCAUCAAAG |
| 2248 | 187 | ACUGGCGCUUUUAUCUGAUUACUUUGAGAGCCAUCACCAGCGACUAUGUCGUAGUGGGU |
| | | AAAGCGCCCUCUUCGGAGGGAAGCAUCAAAG |
| 2249 | 188 | ACUGGCGCUUUUAUCUGAUUACUUUGAGAGCCAUCACCAGCGACUAUGUCGUAGUGGGU |
| | | AAAGCUCACAUGAGGAUCACCCAUGUGAGCAUCAAAG |
| 2250 | 189 | ACUGGCACUUUUACCUGAUUACUUUGAGAGCCAACACCAGCGACUAUGUCGUAGUGGGU |
| | | AAAGCUCCCUCUUCGGAGGGAGCAUCAAAG |
| 2251 | 190 | ACUGGCACUUUUAUCUGAUUACUUUGAGAGCCAUCACCAGCGACUAUGUCGUAGUGGGU |
| | | AAAGCUCCCUCUUCGGAGGGAGCAUCAAAG |
| 2252 | 191 | ACUGGCCCUUUUAUCUGAUUACUUUGAGAGCCAUCACCAGCGACUAUGUCGUAGUGGGU |
| | | AAAGCUCCCUCUUCGGAGGGAGCAUCAAAG |
| 2253 | 192 | ACUGGCGCUUUUACCUGAUUACUUUGAGAGCCAUCACCAGCGACUAUGUCGUAGUGGGU |
| | | AAAGCUCCCUCUUCGGAGGGAGCAUCAAAG |
| 2254 | 193 | ACUGGCGCUUUUAUCUGAUUACUUUGAGAGCCAACACCAGCGACUAUGUCGUAGUGGGU |
| | | AAAGCUCCCUCUUCGGAGGGAGCAUCAAAG |
| 2255 | 195 | ACUGGCACCUUUACCUGAUUACUUUGAGAGCCAACACCAGCGACUAUGUCGUAUGGGUA |
| | | AAGCGCUUACGGACUUCGGUCCGUAAGAAGCAUCAAAG |
| 2256 | 196 | ACUGGCACCUUUAUCUGAUUACUUUGAGAGCCAUCACCAGCGACUAUGUCGUAUGGGUA |
| | | AAGCGCUUACGGACUUCGGUCCGUAAGAAGCAUCAAAG |
| 2257 | 197 | ACUGGCCCCUUUAUCUGAUUACUUUGAGAGCCAUCACCAGCGACUAUGUCGUAUGGGUA |
| | | AAGCGCUUACGGACUUCGGUCCGUAAGAAGCAUCAAAG |
| 2258 | 198 | ACUGGCGCCUUUAUCUGAUUACUUUGAGAGCCAACACCAGCGACUAUGUCGUAUGGGUA |
| | | AAGCGCUUACGGACUUCGGUCCGUAAGAAGCAUCAAAG |
| 2259 | 199 | GCUGGCGCUUUUAUCUGAUUACUUUGAGAGCCAUCACCAGCGACUAUGUCGUAGUGGGU |
| | | AAAGCUCCCUCUUCGGAGGGAGCAUCAAAG |
| 2260 | 200 | GACUGGCGCUUUUAUCUGAUUACUUUGAGAGCCAUCACCAGCGACUAUGUCGUAGUGGG |
| | | UAAAGCUCCCUCUUCGGAGGGAGCAUCAAAG |
| 2261 | 201 | ACUGGCGCCUUUAUCUGAUUACUUUGGAGAGCCAUCACCAGCGACUAUGUCGUAGUGGG |
| | | UAAAGCUCCCUCUUCGGAGGGAGCAUCAAAG |
| 2262 | 202 | ACUGGCGCAUUUAUCUGAUUACUUUGUGAGCCAUCACCAGCGACUAUGUCGUAGUGGGU |
| | | AAAGCUCCCUCUUCGGAGGGAGCAUCAAAG |
| 2263 | 203 | ACUGGCGCCUUUAUCUGAUUACUUUGAGAGCCAUCACCAGCGACUAUGUCGUAGUGGGU |
| | | AAAGCUCCCUCUUCGGAGGGAGCAUCAAAG |
| 2264 | 204 | ACUGGCGCUUUUAUCUGAUUACUUUGGAGAGCCAUCACCAGCGACUAUGUCGUAGUGGG |
| | | UAAAGCUCCCUCUUCGGAGGGAGCAUCAAAG |
| 2265 | 205 | ACUGGCGCAUUUAUCUGAUUACUUUGAGAGCCAUCACCAGCGACUAUGUCGUAGUGGGU |
| | | AAAGCUCCCUCUUCGGAGGGAGCAUCAAAG |
| 2266 | 206 | ACUGGCGCUUUUAUCUGAUUACUUUGUGAGCCAUCACCAGCGACUAUGUCGUAGUGGGU |
| | | AAAGCUCCCUCUUCGGAGGGAGCAUCAAAG |
| 2267 | 207 | ACUGGCGCUUUUAUUCUGAUUACUUUGAGAGCCAUCACCAGCGACUAUGUCGUAGUGGG |
| | | UAAAGCUCCCUCUUCGGAGGGAGCAUCAAAG |
| 2268 | 208 | ACGGCGCUUUUAUCUGAUUACUUUGAGAGCCAUCACCAGCGACUAUGUCGUAGUGGGUA |
| | | AAGCUCCCUCUUCGGAGGGAGCAUCAAAG |
| 2269 | 209 | ACUGGCGCUUUUAUAUGAUUACUUUGAGAGCCAUCACCAGCGACUAUGUCGUAGUGGGU |
| | | AAAGCUCCCUCUUCGGAGGGAGCAUCAAAG |
| 2270 | 210 | ACUGGCGCUUUUAUCUUGAUUACUUUGAGAGCCAUCACCAGCGACUAUGUCGUAGUGGG |
| | | UAAAGCUCCCUCUUCGGAGGGAGCAUCAAAG |
| 2271 | 211 | ACUGGCGCUUUUAUCUGAUUACUUUGAGAGCCAGCACCAGCGACUAUGUCGUAGUGGGU |
| | | AAAGCUCCCUCUUCGGAGGGAGCAUCAAAG |
| 2272 | 212 | ACUGGCGCUGUUAUCUGAUUACUUCGAGAGCCAUCACCAGCGACUAUGUCGUAGUGGGU |
| | | AAAGCUCCCUCUUCGGAGGGAGCAUCGAAG |
| 2273 | 213 | ACUGGCGCUCUUAUCUGAUUACUUCGAGAGCCAUCACCAGCGACUAUGUCGUAGUGGGU |
| | | AAAGCUCCCUCUUCGGAGGGAGCAUCGAAG |
| 2274 | 214 | ACUGGCGCUUGUAUCUGAUUACUCUGAGAGCCAUCACCAGCGACUAUGUCGUAGUGGGU |
| | | AAAGCUCCCUCUUCGGAGGGAGCAUCAGAG |
| 2275 | 215 | ACUGGCGCUUCUAUCUGAUUACUCUGAGAGCCAUCACCAGCGACUAUGUCGUAGUGGGU |
| | | AAAGCUCCCUCUUCGGAGGGAGCAUCAGAG |
| 2276 | 216 | ACUGGCGCUUUGAUCUGAUUACCUUGAGAGCCAUCACCAGCGACUAUGUCGUAGUGGGU |
| | | AAAGCUCCCUCUUCGGAGGGAGCAUCAAGG |
| 2277 | 217 | ACUGGCGCUUUCAUCUGAUUACCUUGAGAGCCAUCACCAGCGACUAUGUCGUAGUGGGU |
| | | AAAGCUCCCUCUUCGGAGGGAGCAUCAAGG |
| 2278 | 218 | ACUGGCGCUGUUAUCUGAUUACUUUGAGAGCCAUCACCAGCGACUAUGUCGUAGUGGGU |
| | | AAAGCUCCCUCUUCGGAGGGAGCAUCAAAG |
| 2279 | 219 | ACUGGCGCUUUUAUCUGAUUACUUUGAGAGCCAUCACCAGCGACUAUGUCGUAGUGGGU |
| | | AAAGCUCCCUCUUCGGAGGGAGCAUCGAAG |
| 2280 | 220 | ACUGGCGCUUUUAUCUGAUUACUUCGAGAGCCAUCACCAGCGACUAUGUCGUAGUGGGU |
| | | AAAGCUCCCUCUUCGGAGGGAGCAUCAAAG |
| 2281 | 221 | ACUGGCACUUCUAUCUGAUUACUCUGAGAGCCAUCACCAGCGACUAUGUCGUAUGGGUA |
| | | AAGCCGCUUACGGACUUCGGUCCGUAAGAGGCAUCAGAG |
| 2282 | 222 | ACUGGCACUUCUAUCUGAUUACUCUGAGAGCCAUCACCAGCGACUAUGUCGUAGUGGGU |
| | | AAAGCUCCCUCUUCGGAGGGAGCAUCAGAG |
| 2283 | 223 | ACUGGCACCUUUAUCUGAUUACUUUGAGAGCCAUCACCAGCGACUAUGUCGUAUGGGUA |
| | | AAGCCGCUUACGGACUUCGGUCCGUAAGAGGCAUCAAAG |
| 2284 | 224 | ACUGGCACUUGUAUCUGAUUACUCUGAGAGCCAUCACCAGCGACUAUGUCGUAUGGGUA |
| | | AAGCCGCUUACGGACUUCGGUCCGUAAGAGGCAUCAGAG |
| 2285 | 225 | ACUGGCACUUGUAUCUGAUUACUCUGAGAGCCAUCACCAGCGACUAUGUCGUAGUGGGU |
| | | AAAGCUCCCUCUUCGGAGGGAGCAUCAGAG |
| 2287 | 230 | ACUGGCACUUCUAUCUGAUUACUCUGAGAGCCAUCACCAGCGACUAUGUCGUAUGGGUA |
| | | AAGCGCUUACGGACUUCGGUCCGUAAGAAGCAUCAGAG |
| 2288 | 231 | ACUGGCGCUUCUAUCUGAUUACUCUGAGAGCCAUCACCAGCGACUAUGUCGUAUGGGUA |
| | | AAGCCGCUUACGGACUUCGGUCCGUAAGAGGCAUCAGAG |
| 2289 | 232 | ACUGGCACUUCUAUCUGAUUACUCUGAGCGCCAUCACCAGCGACUAUGUCGUAUGGGUA |
| | | AAGCCGCUUACGGACUUCGGUCCGUAAGAGGCAUCAGAG |
| 2290 | 233 | ACUGGCGCUUCUAUCUGAUUACUCUGAGCGCCAUCACCAGCGACUAUGUCGUAUGGGUA |
| | | AAGCCGCUUACGGACUUCGGUCCGUAAGAGGCAUCAGAG |
| 2291 | 234 | ACUGGCGCUUCUAUCUGAUUACUCUGAGCGCCAUCACCAGCGACUAUGUCGUAUGGGUA |
| | | AAGCGCCUUACGGACUUCGGUCCGUAAGGAGCAUCAGAG |
| 2292 | 235 | ACUGGCGCUUCUAUCUGAUUACUCUGAGCGCCAUCACCAGCGACUAUGUCGUAGUGGGU |
| | | AAAGCCGCUUACGGACUUCGGUCCGUAAGAGGCAUCAGAG |
| 2293 | 236 | ACGGGACUUUCUAUCUGAUUACUCUGAAGUCCCUCACCAGCGACUAUGUCGUAUGGGUA |
| | | AAGCCGCUUACGGACUUCGGUCCGUAAGAGGCAUCAGAG |
| 2294 | 237 | ACCUGUAGUUCUAUCUGAUUACUCUGACUACAGUCACCAGCGACUAUGUCGUAUGGGUA |
| | | AAGCCGCUUACGGACUUCGGUCCGUAAGAGGCAUCAGAG |
| 2295 | 238 | ACUGGCGCUUUUAUCUGAUUACUUUGAGAGCCAUCACCAGCGACUAUGUCGUAGUGGGU |
| | | AAAGCUGCACGGUGGGCGCAGCUUCGGCUGACGGUACACCGUGCAGCAUCAAAG |
| 2296 | 239 | ACUGGCGCUUUUAUCUGAUUACUUUGAGAGCCAUCACCAGCGACUAUGUCGUAGUGGGU |
| | | AAAGCUGCACGGUGGGCGCAGCUUCGGCUGACGGUACACCGGUGGGCGCAGCUUCGGCU |
| | | GACGGUACACCGUGCAGCAUCAAAG |
| 2297 | 240 | ACUGGCGCUUUUAUCUGAUUACUUUGAGAGCCAUCACCAGCGACUAUGUCGUAGUGGGU |
| | | AAAGCUGCACGGUGGGCGCAGCUUCGGCUGACGGUACACCGGUGGGCGCAGCUUCGGCU |
| | | GACGGUACACCGGUGGGCGCAGCUUCGGCUGACGGUACACCGUGCAGCAUCAAAG |
| 2298 | 241 | ACUGGCGCUUUUAUCUGAUUACUUUGAGAGCCAUCACCAGCGACUAUGUCGUAGUGGGU |
| | | AAAGCUGCACGGUGGGCGCAGCUUCGGCUGACGGUACACCGGUGGGCGCAGCUUCGGCU |
| | | GACGGUACACCGGUGGGCGCAGCUUCGGCUGACGGUACACCGGUGGGCGCAGCUUCGGC |
| | | UGACGGUACACCGUGCAGCAUCAAAG |
| 2299 | 242 | ACUGGCGCUUUUAUCUGAUUACUUUGAGAGCCAUCACCAGCGACUAUGUCGUAGUGGGU |
| | | AAAGCUGCACGGUGGGCGCAGCUUCGGCUGACGGUACACCGGUGGGCGCAGCUUCGGCU |
| | | GACGGUACACCGGUGGGCGCAGCUUCGGCUGACGGUACACCGGUGGGCGCAGCUUCGGC |
| | | UGACGGUACACCGGUGGGCGCAGCUUCGGCUGACGGUACACCGUGCAGCAUCAAAG |
| 2300 | 243 | ACUGGCGCUUUUAUCUGAUUACUUUGAGAGCCAUCACCAGCGACUAUGUCGUAGUGGGU |
| | | AAAGCUGCACCUAGCGGAGGCUAGGUGCAGCAUCAAAG |
| 2301 | 244 | ACUGGCGCUUUUAUCUGAUUACUUUGAGAGCCAUCACCAGCGACUAUGUCGUAGUGGGU |
| | | AAAGCUGCACCUCGGCUUGCUGAAGCGCGCACGGCAAGAGGCGAGGUGCAGCAUCAAAG |
| 2302 | 245 | ACUGGCGCUUUUAUCUGAUUACUUUGAGAGCCAUCACCAGCGACUAUGUCGUAGUGGGU |
| | | AAAGCUGCACCUCUCUCGACGCAGGACUCGGCUUGCUGAAGCGCGCACGGCAAGAGGCG |
| | | AGGGGCGGCGACUGGUGAGUACGCCAAAAAUUUUGACUAGCGGAGGCUAGAAGGAGAGA |
| | | GGUGCAGCAUCAAAG |
| 2303 | 246 | ACUGGCGCUUUUAUCUGAUUACUUUGAGAGCCAUCACCAGCGACUAUGUCGUAGUGGGU |
| | | AAAGCUGCACGGUGCCCGUCUGUUGUGUCGAGAGACGCCAAAAAUUUUGACUAGCGGAG |
| | | GCUAGAAGGAGAGAGAUGGGUGCCGUGCAGCAUCAAAG |
| 2304 | 247 | ACUGGCGCUUUUAUCUGAUUACUUUGAGAGCCAUCACCAGCGACUAUGUCGUAGUGGGU |
| | | AAAGCUGCACAUGGAGAGGAGAUGUGCAGCAUCAAAG |
| 2305 | 248 | ACUGGCGCUUUUAUCUGAUUACUUUGAGAGCCAUCACCAGCGACUAUGUCGUAGUGGGU |
| | | AAAGCUGCACAUGGAGAUGUGCAGCAUCAAAG |
| 2306 | 249 | ACUGGCGCUUUUAUCUGAUUACUUUGAGAGCCAUCACCAGCGACUAUGUCGUAGUGGGU |
| | | AAAGCUUGGGCGCAGCGUCAAUGACGCUGACGGUACAAGCAUCAAAG |
| 2307 | 250 | ACUGGCGCUUUUAUCUGAUUACUUUGAGAGCCAUCACCAGCGACUAUGUCGUAGUGGGU |
| | | AAAGCUGCACUAUGGGCGCAGCGUCAAUGACGCUGACGGUACAGGCCACAUGAGGAUCA |
| | | CCCAUGUGGUAUAGUGCAGCAUCAAAG |
| 2308 | 251 | ACUGGCGCUUUUAUCUGAUUACUUUGAGAGCCAUCACCAGCGACUAUGUCGUAGUGGGU |
| | | AAAGCUGCACUAUGGGCGCAGCUCAUGAGGAUCACCCAUGAGCUGACGGUACAGGCCAC |
| | | AUGAGGAUCACCCAUGUGGUAUAGUGCAGCAUCAAAG |
| 2309 | 252 | ACUGGCGCUUUUAUCUGAUUACUUUGAGAGCCAUCACCAGCGACUAUGUCGUAGUGGGU |
| | | AAAGCUGCACUAUGGGCGCAGCGUCAAUGACGCUGACGGUACAGGCCACAUGGCAGUCG |
| | | UAACGACGCGGGUGGUAUAGUGCAGCAUCAAAG |
| 2310 | 253 | ACUGGCGCUUUUAUCUGAUUACUUUGAGAGCCAUCACCAGCGACUAUGUCGUAGUGGGU |
| | | AAAGCUGCACUAUGGGCGCAGCAAACAUGGCAGUCCUAAGGACGCGGGUUUUGCUGACG |
| | | GUACAGGCCACAUGGCAGUCGUAACGACGCGGGUGGUAUAGUGCAGCAUCAAAG |
| 2311 | 254 | ACUGGCGCUUUUAUCUGAUUACUUUGAGAGCCAUCACCAGCGACUAUGUCGUAGUGGGU |
| | | AAAGCUGCACUAUGGGCGCAGACAUGGCAGUCGUAACGACGCGGGUCUGACGGUACAGG |
| | | CCACAUGAGGAUCACCCAUGUGGUAUAGUGCAGCAUCAAAG |
| 2312 | 255 | ACUGGCGCUUUUAUCUGAUUACUUUGAGAGCCAUCACCAGCGACUAUGUCGUAGUGGGU |
| | | AAAGCUGCACUAAGGAGUUUAUAUGGAAACCCUUAGUGCAGCAUCAAAG |
| 2313 | 256 | ACUGGCGCUUUUAUCUGAUUACUUUGAGAGCCAUCACCAGCGACUAUGUCGUAGUGGGU |
| | | AAAGCUCAGGAAGCACUAUGGGCGCAGCGUCAAUGACGCUGACGGUACAGGCCAGACAA |
| | | UUAUUGUCUGGUAUAGUGCAGCAGCAGAACAAUUUGCUGAGGGCUAUUGAGGCGCAACA |
| | | GCAUCUGUUGCAACUCACAGUCUGGGGCAUCAAGCAGCUCCAGGCAAGAAUCCUGAGCA |
| | | UCAAAG |
| 2314 | 257 | ACUGGCGCUUUUAUCUGAUUACUUUGAGAGCCAUCACCAGCGACUAUGUCGUAGUGGGU |
| | | AAAGCUGCACGCCCUGAAGAAGGGCGUGCAGCAUCAAAG |
| 2315 | 258 | ACUGGCGCUUUUAUCUGAUUACUUUGAGAGCCAUCACCAGCGACUAUGUCGUAGUGGGU |
| | | AAAGCUGCACGGCUCGUGUAGCUCAUUAGCUCCGAGCCGUGCAGCAUCAAAG |
| 2316 | 259 | ACUGGCGCUUUUAUCUGAUUACUUUGAGAGCCAUCACCAGCGACUAUGUCGUAGUGGGU |
| | | AAAGCUGCACCCGUGUGCAUCCGCAGUGUCGGAUCCACGGGUGCAGCAUCAAAG |
| 2317 | 260 | ACUGGCGCUUUUAUCUGAUUACUUUGAGAGCCAUCACCAGCGACUAUGUCGUAGUGGGU |
| | | AAAGCUGCACGGAAUCCAUUGCACUCCGGAUUUCACUAGGUGCAGCAUCAAAG |
| 2318 | 261 | ACUGGCGCUUUUAUCUGAUUACUUUGAGAGCCAUCACCAGCGACUAUGUCGUAGUGGGU |
| | | AAAGCUGCACAUGCAUGUCUAAGACAGCAUGUGCAGCAUCAAAG |
| 2319 | 262 | ACUGGCGCUUUUAUCUGAUUACUUUGAGAGCCAUCACCAGCGACUAUGUCGUAGUGGGU |
| | | AAAGCUGCACAAAACAUAAGGAAAACCUAUGUUGUGCAGCAUCAAAG |
| 2320 | 263 | ACUGGCGCUUCUAUCUGAUUACUCUGAGCGCCAUCACCAGCGACUAUGUCGUAGUGGGU |
| | | AAAGCCGCUUACGGACUAUGGGCGCAGCGUCAAUGACGCUGACGGUACAGGCCAGACAA |
| | | UUAUUGUCUGGUAUAGUCCGUAAGAGGCAUCAGAG |
| 2321 | 264 | ACUGGCGCUUCUAUCUGAUUACUCUGAGCGCCAUCACCAGCGACUAUGUCGUAGUGGGU |
| | | AAAGCCGCUUACGGGUGGGCGCAGCGUCAAUGACGCUGACGGUACAGGCCAGACAAUUA |
| | | UUGUCUGGUACCCGUAAGAGGCAUCAGAG |
| 2322 | 265 | ACUGGCGCUUCUAUCUGAUUACUCUGAGCGCCAUCACCAGCGACUAUGUCGUAGUGGGU |
| | | AAAGCCGCUUACGGACUAUGGGCGCAGCGUCAAUGACGCUGACGGUACAGGCCACAUGA |
| | | GGAUCACCCAUGUGGUAUAGUCCGUAAGAGGCAUCAGAG |
| 2323 | 266 | ACUGGCGCUUUUAUCUGAUUACUUUGAGAGCCAUCACCAGCGACUAUGUCGUAGUGGGU |
| | | AAAGCUCCCUAUGGGCGCAGCGUCAAUGACGCUGACGGUACAGGCCACAUGAGGAUCAC |
| | | CCAUGUGGUAUAGGGAGCAUCAAAG |
| 2324 | 267 | ACUGGCGCUUCUAUCUGAUUACUCUGAGCGCCAUCACCAGCGACUAUGUCGUAGUGGGU |
| | | AAAGCCGCUUACGGACUAUGGGCGCAGCUCAUGAGGAUCACCCAUGAGCUGACGGUACA |
| | | GGCCACAUGAGGAUCACCCAUGUGGUAUAGUCCGUAAGAGGCAUCAGAG |
| 2325 | 268 | ACUGGCGCUUUUAUCUGAUUACUUUGAGAGCCAUCACCAGCGACUAUGUCGUAGUGGGU |
| | | AAAGCUCCCUAUGGGCGCAGCUCAUGAGGAUCACCCAUGAGCUGACGGUACAGGCCACA |
| | | UGAGGAUCACCCAUGUGGUAUAGGGAGCAUCAAAG |
| 2326 | 269 | ACUGGCGCUUCUAUCUGAUUACUCUGAGCGCCAUCACCAGCGACUAUGUCGUAGUGGGU |
| | | AAAGCCGCUUACGGACUAUGGGCGCAGCGUCAAUGACGCUGACGGUACAGGCCACAUGG |
| | | CAGUCGUAACGACGCGGGUGGUAUAGUCCGUAAGAGGCAUCAGAG |
| 2327 | 270 | ACUGGCGCUUUUAUCUGAUUACUUUGAGAGCCAUCACCAGCGACUAUGUCGUAGUGGGU |
| | | AAAGCUCCCUAUGGGCGCAGCGUCAAUGACGCUGACGGUACAGGCCACAUGGCAGUCGU |
| | | AACGACGCGGGUGGUAUAGGGAGCAUCAAAG |
| 2328 | 27 | ACUGGCGCUUCUAUCUGAUUACUCUGAGCGCCAUCACCAGCGACUAUGUCGUAGUGGGU |
| | | AAAGCCGCUUACGGACUAUGGGCGCAGCAAACAUGGCAGUCCUAAGGACGCGGGUUUUG |
| | | CUGACGGUACAGGCCACAUGGCAGUCGUAACGACGCGGGUGGUAUAGUCCGUAAGAGGC |
| | | AUCAGAG |
| 2329 | 272 | ACUGGCGCUUUUAUCUGAUUACUUUGAGAGCCAUCACCAGCGACUAUGUCGUAGUGGGU |
| | | AAAGCUCCCUAUGGGCGCAGCAAACAUGGCAGUCCUAAGGACGCGGGUUUUGCUGACGG |
| | | UACAGGCCACAUGGCAGUCGUAACGACGCGGGUGGUAUAGGGAGCAUCAAAG |
| 2330 | 273 | ACUGGCGCUUCUAUCUGAUUACUCUGAGCGCCAUCACCAGCGACUAUGUCGUAGUGGGU |
| | | AAAGCCGCUUACGGACUAUGGGCGCAGACAUGGCAGUCGUAACGACGCGGGUCUGACGG |
| | | UACAGGCCACAUGAGGAUCACCCAUGUGGUAUAGUCCGUAAGAGGCAUCAGAG |
| 2331 | 274 | ACUGGCGCUUUUAUCUGAUUACUUUGAGAGCCAUCACCAGCGACUAUGUCGUAGUGGGU |
| | | AAAGCUCCCUAUGGGCGCAGACAUGGCAGUCGUAACGACGCGGGUCUGACGGUACAGGC |
| | | CACAUGAGGAUCACCCAUGUGGUAUAGGGAGCAUCAAAG |
| 2332 | 275 | ACUGGCGCUUUUAUCUGAUUACUUUGAGAGCCAUCACCAGCGACUAUGUCGUAGUGGGU |
| | | AAAGCUGCACUAUGGGCGCAGCACCUGAGGAUCACCCAGGUGCUGACGGUACAGGCCAC |
| | | CUGAGGAUCACCCAGGUGGUAUAGUGCAGCAUCAAAG |
| 2333 | 276 | ACUGGCGCUUUUAUCUGAUUACUUUGAGAGCCAUCACCAGCGACUAUGUCGUAGUGGGU |
| | | AAAGCUGCACUAUGGGCGCAGCGCAUGAGGAUCACCCAUGCGCUGACGGUACAGGCCGC |
| | | AUGAGGAUCACCCAUGCGGUAUAGUGCAGCAUCAAAG |
| 2334 | 277 | ACUGGCGCUUUUAUCUGAUUACUUUGAGAGCCAUCACCAGCGACUAUGUCGUAGUGGGU |
| | | AAAGCUGCACUAUGGGCGCAGCGCCUGAGGAUCACCCAGGCGCUGACGGUACAGGCCGC |
| | | CUGAGGAUCACCCAGGCGGUAUAGUGCAGCAUCAAAG |
| 2335 | 278 | ACUGGCGCUUUUAUCUGAUUACUUUGAGAGCCAUCACCAGCGACUAUGUCGUAGUGGGU |
| | | AAAGCUGCACUAUGGGCGCAGCGCCUGAGCAUCAGCCAGGCGCUGACGGUACAGGCCGC |
| | | CUGAGCAUCAGCCAGGCGGUAUAGUGCAGCAUCAAAG |
| 2336 | 279 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGCAUCAGCCAUGUGCUGACGGUACAGGCCAC |
| | | AUGAGCAUCAGCCAUGUGGUAUAGUGCAGCAUCAAAG |
| 2337 | 280 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGUAUCAACCAUGUGCUGACGGUACAGGCCAC |
| | | AUGAGUAUCAACCAUGUGGUAUAGUGCAGCAUCAAAG |
| 2338 | 281 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGAAUCAGCCAUGUGCUGACGGUACAGGCCAC |
| | | AUGAGAAUCAGCCAUGUGGUAUAGUGCAGCAUCAAAG |
| 2339 | 282 | ACUGGCGCUUUUAUCUGAUUACUUUGAGAGCCAUCACCAGCGACUAUGUCGUAGUGGGU |
| | | AAAGCUGCACUAUGGGCGCAGCCCUUGAGGAUCACCCAUGUGCUGACGGUACAGGCCCC |
| | | UUGAGGAUCACCCAUGUGGUAUAGUGCAGCAUCAAAG |
| 2340 | 283 | ACUGGCGCUUUUAUCUGAUUACUUUGAGAGCCAUCACCAGCGACUAUGUCGUAGUGGGU |
| | | AAAGCUGCACUAUGGGCGCAGCACUUGAGGAUCACCCAUGUGCUGACGGUACAGGCCAC |
| | | UUGAGGAUCACCCAUGUGGUAUAGUGCAGCAUCAAAG |
| 2341 | 284 | ACUGGCGCUUUUAUCUGAUUACUUUGAGAGCCAUCACCAGCGACUAUGUCGUAGUGGGU |
| | | AAAGCUGCACUAUGGGCGCAGCACCUGAGGAUCACCCAUGUGCUGACGGUACAGGCCAC |
| | | CUGAGGAUCACCCAUGUGGUAUAGUGCAGCAUCAAAG |
| 2342 | 285 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGAUCACCUAUGUGCUGACGGUACAGGCCAC |
| | | AUGAGGAUCACCUAUGUGGUAUAGUGCAGCAUCAAAG |
| 2343 | 286 | ACUGGCGCUUUUAUCUGAUUACUUUGAGAGCCAUCACCAGCGACUAUGUCGUAGUGGGU |
| | | AAAGCUGCACUAUGGGCGCAGCACAUUAGGAUCACCAAUGUGCUGACGGUACAGGCCAC |
| | | AUUAGGAUCACCAAUGUGGUAUAGUGCAGCAUCAAAG |
| 2344 | 287 | ACUGGCGCUUUUAUCUGAUUACUUUGAGAGCCAUCACCAGCGACUAUGUCGUAGUGGGU |
| | | AAAGCUGCACUAUGGGCGCAGCACAUUAGGAUCACCGAUGUGCUGACGGUACAGGCCAC |
| | | AUUAGGAUCACCGAUGUGGUAUAGUGCAGCAUCAAAG |
| 2345 | 288 | ACUGGCGCUUUUAUCUGAUUACUUUGAGAGCCAUCACCAGCGACUAUGUCGUAGUGGGU |
| | | AAAGCUGCACUAUGGGCGCAGCACAUUAGGAUCACCUAUGUGCUGACGGUACAGGCCAC |
| | | AUUAGGAUCACCUAUGUGGUAUAGUGCAGCAUCAAAG |
| 2346 | 289 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGAUUACCCAUGUGCUGACGGUACAGGCCAC |
| | | AUGAGGAUUACCCAUGUGGUAUAGUGCAGCAUCAAAG |
| 2347 | 290 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGAUAACCCAUGUGCUGACGGUACAGGCCAC |
| | | AUGAGGAUAACCCAUGUGGUAUAGUGCAGCAUCAAAG |
| 2348 | 291 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGAUGACCCAUGUGCUGACGGUACAGGCCAC |
| | | AUGAGGAUGACCCAUGUGGUAUAGUGCAGCAUCAAAG |
| 2349 | 292 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGACCACCCAUGUGCUGACGGUACAGGCCAC |
| | | AUGAGGACCACCCAUGUGGUAUAGUGCAGCAUCAAAG |
| 2350 | 293 | ACUGGCGCUUUUAUCUGAUUACUUUGAGAGCCAUCACCAGCGACUAUGUCGUAGUGGGU |
| | | AAAGCUGCACUAUGGGCGCAGCAGAUGAGGAUCACCCAUGGGCUGACGGUACAGGCCAG |
| | | AUGAGGAUCACCCAUGGGGUAUAGUGCAGCAUCAAAG |
| 2351 | 294 | ACUGGCGCUUUUAUCUGAUUACUUUGAGAGCCAUCACCAGCGACUAUGUCGUAGUGGGU |
| | | AAAGCUGCACUAUGGGCGCAGCACAUGGGGAUCACCCAUGUGCUGACGGUACAGGCCAC |
| | | AUGGGGAUCACCCAUGUGGUAUAGUGCAGCAUCAAAG |
| 2352 | 295 | ACUGGCGCUUUUAUCUGAUUACUUUGAGAGCCAUCACCAGCGACUAUGUCGUAGUGGGU |
| | | AAAGCUGCACUAUGGGCGCAGCACAUGAGGAUCACCCAUGUGCUGACGGUACAGGCCAC |
| | | AUGAGGAUCACCCAUGUGGUAUAGUGCAGCAUCAAAG |
| 9588 | 316 | ACUGGCGCUUCUAUCUGAUUACUCUGAGCGCCAUCACCAGCGACUAUGUCGUAGUGGGU |
| | | AAAGCUCCCUCUUCGGAGGGAGCAUCAGAG |
| 2376 | 320 | ACUGGGCCUUCUAUCUGAUUACUCUGAGGCCCAUCACCAGCGACUAUGUCGUAGUGGGU |
| | | AAAGCCGCUUAGGGACUUCGGUCCCUAAGAGGCAUCAGAG |
| 2378 | 321 | ACUGGGCCUUCUAUCUGAUUACUCUGAGGCCCAUCACCAGCGACUAUGUCGUAGUGGGU |
| | | AAAGCCCUUAGGGACUUCGGUCCCUAAGGGCAUCAGAG |
| 2382 | 323 | ACUGGGCCUUCUAUCUGAUUACUCUGAGGCCCAUCACCAGCGACUAUGUCGUAGUGGGU |
| | | AAAGCUCCCUCUUCGGAGGGAGCAUCAGAG |
| 2383 | 324 | ACUGGGCCUUCUAUCUGAUUACUCUGAGGCCCAUCACCAGCGACUAUGUCGUAGUGGGU |
| | | AAAGCCCUUAGGGACCUUGGUCCCUAAGGGCAUCAGAG |
| 2384 | 325 | ACUGGGCCUUCUAUCUGAUUACUCUGAGGCCCAUCACCAGCGACUAUGUCGUAGUGGGU |
| | | AAAGCCUCUUAGGGACCUUGGUCCCUAAGAGGCAUCAGAG |
| 2385 | 326 | ACUGGGCCUUCUAUCUGAUUACUCUGAGGCCCAUCACCAGCGACUAUGUCGUAGUGGGU |
| | | AAAGCUCCCUCCUUGGAGGGAGCAUCAGAG |
| 2386 | 327 | ACUGGGCCUUCUAUCUGAUUACUCUGAGGCCCAUCACCAGCGACUAUGUCGUAGUGGGU |
| | | AAAGCCCUUAGGGAGGAAACUCCCUAAGGGCAUCAGAG |
| 2387 | 328 | ACUGGGCCUUCUAUCUGAUUACUCUGAGGCCCAUCACCAGCGACUAUGUCGUAGUGGGU |
| | | AAAGCCUCUUAGGGAGGAAACUCCCUAAGAGGCAUCAGAG |
| 2388 | 329 | ACUGGGCCUUCUAUCUGAUUACUCUGAGGCCCAUCACCAGCGACUAUGUCGUAGUGGGU |
| | | AAAGCUCCCUGGAAACAGGGAGCAUCAGAG |
| 2389 | 330 | ACUGGGCCUUCUAUCUGAUUACUCUGAGGCCCAUCACCAGCGACUAUGUCGUAGUGGGU |
| | | AAAGCCCUUAGGGACUUCAGGUCCCUAAGGGCAUCAGAG |
| 2390 | 331 | ACUGGGCCUUCUAUCUGAUUACUCUGAGGCCCAUCACCAGCGACUAUGUCGUAGUGGGU |
| | | AAAGCCUCUUAGGGACUUCAGGUCCCUAAGAGGCAUCAGAG |
| 2391 | 332 | ACUGGGCCUUCUAUCUGAUUACUCUGAGGCCCAUCACCAGCGACUAUGUCGUAGUGGGU |
| | | AAAGCUCCCUCUUCAGGAGGGAGCAUCAGAG |
| 2392 | 333 | ACUGGGCCUUCUAUCUGAUUACUCUGAGGCCCAUCACCAGGCACUAUGUGCUAGUGGGU |
| | | AAAGCCCUUAGGGACCUUGGUCCCUAAGGGCAUCAGAG |
| 2393 | 334 | ACUGGGCCUUCUAUCUGAUUACUCUGAGGCCCAUCACCAGGCACUAUGUGCUAGUGGGU |
| | | AAAGCCUCUUAGGGACCUUGGUCCCUAAGAGGCAUCAGAG |
| 2394 | 335 | ACUGGGCCUUCUAUCUGAUUACUCUGAGGCCCAUCACCAGGCACUAUGUGCUAGUGGGU |
| | | AAAGCUCCCUCCUUGGAGGGAGCAUCAGAG |
| 2395 | 336 | ACUGGGCCUUCUAUCUGAUUACUCUGAGGCCCAUCACCAGGCACUAUGUGCUAGUGGGU |
| | | AAAGCCCUUAGGGAGGAAACUCCCUAAGGGCAUCAGAG |
| 2396 | 337 | ACUGGGCCUUCUAUCUGAUUACUCUGAGGCCCAUCACCAGGCACUAUGUGCUAGUGGGU |
| | | AAAGCCUCUUAGGGAGGAAACUCCCUAAGAGGCAUCAGAG |
| 2397 | 338 | ACUGGGCCUUCUAUCUGAUUACUCUGAGGCCCAUCACCAGGCACUAUGUGCUAGUGGGU |
| | | AAAGCUCCCUGGAAACAGGGAGCAUCAGAG |
| 2398 | 339 | ACUGGGCCUUCUAUCUGAUUACUCUGAGGCCCAUCACCAGGCACUAUGUGCUAGUGGGU |
| | | AAAGCCCUUAGGGACUUCAGGUCCCUAAGGGCAUCAGAG |
| 2399 | 340 | ACUGGGCCUUCUAUCUGAUUACUCUGAGGCCCAUCACCAGGCACUAUGUGCUAGUGGGU |
| | | AAAGCCUCUUAGGGACUUCAGGUCCCUAAGAGGCAUCAGAG |
| 2400 | 341 | ACUGGGCCUUCUAUCUGAUUACUCUGAGGCCCAUCACCAGGCACUAUGUGCUAGUGGGU |
| | | AAAGCUCCCUCUUCAGGAGGGAGCAUCAGAG |

Additional gRNA variants are presented in the attached sequence listing, as SEQ ID NOS: 2101-2237 and 9257-9289 and 9588.

In some embodiments, a gRNA variant comprises one or more additional modifications to a sequence of SEQ ID NO:2238, SEQ ID NO:2239, SEQ ID NO:2240, SEQ ID NO:2241, SEQ ID NO:2243, SEQ ID NO:2256, SEQ ID NO:2274, SEQ ID NO:2275, SEQ ID NO:2279, SEQ ID NO:2281, SEQ ID NO: 2285, SEQ ID NO: 2289, SEQ ID NO: 2292, SEQ ID NO: 2308, or 9588 of Table 2.

c. Complex Formation with CasX Protein

In some embodiments, upon expression of the components of the rAAV vector, a gRNA variant of the disclosure has an improved ability to form an RNP complex with a Class 2, Type V protein and bind a target nucleic acid, including CasX variant proteins comprising any one of the sequences SEQ ID NOS: 190, 197, 348, 351, 355, 484, 9382-9542, and 9607-9609, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments of the rAAV vector, upon expression, the gRNA variant is complexed as an RNP with a CasX variant protein comprising any one of the sequences SEQ ID NOS: 190, 197, 348, 351, 355, 484, 9382-9542, or 9607-9609, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto.

In some embodiments of the rAAV vector, a gRNA variant has an improved ability to form a complex with a CasX variant protein when compared to a reference gRNA, thereby improving its ability to form a cleavage-competent ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing of a target nucleic acid.

d. gRNA Scaffold 316

In order to generate a gRNA scaffold with improved characteristics, but that had a scaffold length shorter than 90 nucleotides, a gRNA variant scaffold was designed wherein the gRNA scaffold 174 (SEQ ID NO: 2238) sequence, was modified by introducing one, two, three, four or more mutations at positions selected from the group consisting of U11, U24, A29, and A87. In some embodiments, the gRNA variant comprises a sequence of SEQ ID NO: 2238, or a sequence having at least about 70% sequence identity thereto, and four mutations at positions selected from the group consisting of U11, U24, A29, and A87. In one embodiment of the foregoing, the mutations consist of U11C, U24C, A29C, and A87G, resulting in the gRNA scaffold 316 sequence of SEQ ID NO: 9588, having 89 nucleotides.

In another embodiments, the gRNA sequence was generated wherein the scaffold 235 sequence (SEQ ID NO: 2292) was modified by a domain swap in which the extended stemloop of gRNA scaffold 174 replaced the extended stemloop of the 235 scaffold, resulting in the gRNA scaffold 316 sequence of SEQ ID NO: 9588, having 89 nucleotides in the scaffold, compared with the 99 nucleotides of gRNA scaffold235. The 316 scaffold was determined to perform comparably or more favorably than gRNA scaffold 174 in editing assays, as described in the Examples. The resulting 316 scaffold had the further advantage in that the extended stemloop did not contain CpG motifs; an enhanced property described more fully, below.

e. Complex Formation with CasX Protein

Upon delivery of the rAAV to a target cell and expression of the encoded components, the gRNA variant is capable of complexing as an RNP with a CasX protein and binding to the target nucleic acid. In some embodiments, a gRNA variant has an improved ability to form an RNP complex with a CasX protein when compared to a reference gRNA or another gRNA variant from which it was derived. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing or modification of a target nucleic acid.

IV. CRISPR Proteins of the rAAV

The present disclosure provides rAAV encoding a CRISPR nuclease that have utility in genome editing of eukaryotic cells. In some embodiments, the CRISPR nuclease employed in the genome editing systems is a Class 2, Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and Cas(D. In some embodiments, the present disclosure provides rAAV encoding a CasX variant protein and one or more gRNAs that upon expression in a transfected cell are able to form an RNP complex and modify a target nucleic acid sequence in eukaryotic cells.

The term “CasX protein”, as used herein, refers to a family of proteins, and encompasses all naturally occurring CasX proteins, proteins that share at least 50% identity to naturally occurring CasX proteins, as well as CasX variants possessing one or more improved characteristics relative to a naturally-occurring reference CasX protein, described more fully, below.

The present disclosure provides highly-modified CasX proteins having multiple mutations relative to one or more reference CasX proteins. Any changes in the amino acid sequence of a reference CasX protein which results in a CasX and that leads to an improved characteristic relative to the reference CasX protein is considered a CasX variant protein of the disclosure, provided the CasX retains the ability to form an RNP with a gRNA and retains nuclease activity.

CasX proteins of the disclosure comprise at least one of the following domains: a non-target strand binding (NTSB) domain, a target strand loading (TSL) domain, a helical I domain (which is further divided into helical I-I and I-II subdomains), a helical II domain, an oligonucleotide binding domain (OBD, which is further divided into OBD-I and OBD-II subdomains), and a RuvC DNA cleavage domain (which is further divided into RuvC-I and II subdomains). The RuvC domain may be modified or deleted in a catalytically-dead CasX variant, described more fully, below.

In some embodiments, a CasX variant protein can bind and/or modify (e.g., nick, catalyze a double-strand break, methylate, demethylate, etc.) a target nucleic acid at a specific sequence targeted by an associated gRNA, which hybridizes to a sequence within the target nucleic acid sequence. In some embodiments, the CasX comprises a nuclease domain having double-stranded cleavage activity that generates a double-stranded break within 18-26 nucleotides 5′ of a PAM site on the target strand and 10-18 nucleotides 3′ on the non-target strand, resulting in overhangs that can facilitate a higher degree of editing efficiency or insertion of a donor template nucleic acid by HDR or HITI repair mechanisms of the host cell, compared to other CRISPR systems.

a. Reference CasX Proteins

The disclosure provides naturally-occurring CasX proteins (referred to herein as a “reference CasX protein”), which were subsequently modified to create the CasX variants of the disclosure. For example, reference CasX proteins can be isolated from naturally occurring prokaryotes, such as Deltaproteobacteria, Planctomycetes, or Candidatus sungbacteria species. A reference CasX protein is a type V CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter. In some embodiments, a reference CasX protein comprises a sequence identical to a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes. In some embodiments, a reference CasX protein comprises a sequence identical to a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNENFDDPN LIILPLAFGK RQGREFIWND |
| 601 | LLSLETGSLK LANGRVIEKT LYNRRTRQDE PALEVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria. In some embodiments, a reference CasX protein comprises a sequence identical to a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NOMLPALVKV DEPEEAEESP RLRFENGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHELGEID RVCEKRRQGL VRTPEDSQIR ARYMDIISER |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPODRWK LFFHMVISGP |
| 541 | PPVEYLDFSS DVRSRARTVI GINRGEVNPL AYAVVSVEDG QVLEEGLLGK KEYIDOLIET |
| 601 | RRRISEYQSR EQTPPRDLRQ RVRHLODTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ POWGOFFCHR CGENGQSDVL AATNLARRAI |
| 841 | SLIRRLPDTD TPPTP. |

b. Class 2, Type V CasX Variant Proteins

The present disclosure provides Class 2, Type V, CasX variants of a reference CasX protein or variants derived from other CasX variants (interchangeably referred to herein as “Class 2, Type V CasX variant”, “CasX variant” or “CasX variant protein”) for use in the rAAV, wherein the Class 2, Type V CasX variants comprise at least one modification in at least one domain relative to the reference CasX protein, including but not limited to the sequences of SEQ ID NOS:1-3, or at least one modification relative to another CasX variant. Any change in amino acid sequence of a reference CasX protein or to another CasX variant protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

The CasX variants of the disclosure have one or more improved characteristics compared to a reference CasX protein of SEQ ID NO:1, SEQ ID NO:2 or SEQ ID NO:3. Exemplary improved characteristics are described in WO2020247882A1 and PCT/US20/36505, incorporated by reference herein.

Exemplary improved characteristics of the CasX variant embodiments include, but are not limited to improved folding of the variant, increased binding affinity to the gRNA, increased binding affinity to the target nucleic acid, improved ability to utilize a greater spectrum of PAM sequences in the editing and/or binding of target nucleic acid, improved unwinding of the target DNA, improved editing activity, improved editing efficiency, improved editing specificity for the target nucleic acid, improved specificity ratio for the target nucleic acid, decreased off-target editing or cleavage, increased percentage of a eukaryotic genome that can be efficiently edited, increased activity of the nuclease, increased target strand loading for double strand cleavage, decreased target strand loading for single strand nicking, increased binding of the non-target strand of DNA, improved protein stability, improved protein:gRNA (RNP) complex stability, and improved fusion characteristics. In particular, the CasX variant proteins of the disclosure have an enhanced ability to efficiently edit and/or bind target DNA, when complexed with a guide RNA scaffold as an RNP, utilizing a PAM TC motif, including PAM sequences selected from TTC, ATC, GTC, or CTC, compared to an RNP of a reference CasX protein and a reference gRNA. In the foregoing, the PAM sequence is located at least 1 nucleotide 5′ to the non-target strand of the protospacer having identity with the targeting sequence of the gRNA in an assay system compared to the editing efficiency and/or binding of an RNP comprising the reference CasX protein and reference gRNA in a comparable assay system. In the foregoing embodiments, the one or more of the improved characteristics of the CasX variant is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3. In some embodiments, an RNP comprising the CasX variant protein and a gRNA variants of the disclosure, at a concentration of 20 pM or less, is capable of cleaving a double stranded DNA target with an efficiency of at least 80%. In some embodiments, the RNP at a concentration of 20 pM or less is capable of cleaving a double stranded DNA target with an efficiency of at least 40%, at least 50%, at least 60%, at least 70%, at least 80%, at least 85%, at least 90% or at least 95%. In some embodiments, the RNP at a concentration of 50 pM or less, 40 pM or less, 30 pM or less, 20 pM or less, 10 pM or less, or 5 pM or less, is capable of cleaving a double stranded DNA target with an efficiency of at least 40%, at least 50%, at least 60%, at least 70%, at least 80%, at least 85%, at least 90% or at least 95%. These improved characteristics are described in more detail, below.

In some embodiments, the modification of the CasX variant is a mutation in one or more amino acids of the reference CasX. In other embodiments, the modification is an insertion or substitution of a part or all of a domain from a different CasX protein. Mutations can be introduced in any one or more domains of the reference CasX protein or in a CasX variant to result in a CasX variant, and may include, for example, deletion of part or all of one or more domains, or one or more amino acid substitutions, deletions, or insertions in any domain of the reference CasX protein or the CasX variant from which it was derived.

In other embodiments, the disclosure provides CasX variants wherein the CasX variants comprise one or more modifications relative to another CasX variant; e.g., CasX variant 515 and 527 is a variant of CasX variant 491 and CasX variants 668 and 672 are variants of CasX 535.

In some embodiments, a CasX variant protein comprises between 500 and 1500 amino acids, between 700 and 1200 amino acids, between 800 and 1100 amino acids, or between 900 and 1000 amino acids.

c. CasX Variant Proteins with Domains from Multiple Source Proteins

Also contemplated within the scope of the disclosure are chimeric CasX proteins for use in the rAAV. As used herein, a “chimeric CasX” protein refers to both a CasX protein containing at least two domains from different sources, as well a CasX protein containing at least one domain that itself is chimeric. Accordingly, in some embodiments, a chimeric CasX protein is one that includes at least two domains isolated or derived from different sources, such as from two different naturally occurring CasX proteins, (e.g., from two different CasX reference proteins), or from two different CasX variant proteins. In other embodiments, the chimeric CasX protein is one that contains at least one domain that is a chimeric domain, e.g., in some embodiments, part of a domain comprises a substitution from a different CasX protein (from a reference CasX protein, or another CasX variant protein).

In some embodiments, a CasX variant protein of the disclosure comprises a modification, and the modification is an insertion or substitution of a part or all of a domain from a different CasX protein. In particular embodiments, the CasX variants 514-840 and SEQ ID NOS: 9382-9542 and 9607-9609 have a NTSB and helical 1-I domain derived from the sequence of SEQ ID NO: 1, while the other domains are derived from SEQ ID NO: 2, it being understood that the variants may have 1, 2, 3, 4 or more amino acid changes at select locations. In one embodiment, the CasX variant of 494 has a NTSB domain derived from the sequence of SEQ ID NO: 1, while the other domains are derived from SEQ ID NO: 2.

In some embodiments, a CasX variant protein for use in the rAAV comprises at least one chimeric domain comprising a first part from a first CasX protein and a second part from a second, different CasX protein. As used herein, a “chimeric domain” refers to a domain containing at least two parts isolated or derived from different sources, such as two naturally occurring proteins or portions of domains from two reference CasX proteins, or even portions of two CasX variant proteins. The at least one chimeric domain can be any of the NTSB, TSL, helical I, helical II, OBD or RuvC domains as described herein. As an example of the foregoing, a chimeric RuvC domain comprises amino acids 660 to 823 of SEQ ID NO: 1 and amino acids 921 to 978 of SEQ ID NO: 2. As an alternative example of the foregoing, a chimeric RuvC domain comprises amino acids 647 to 810 of SEQ ID NO: 2 and amino acids 934 to 986 of SEQ ID NO: 1. In the case of split or non-contiguous domains such as helical I, RuvC and OBD, a portion of the non-contiguous domain can be replaced with the corresponding portion from any other source. For example, the helical I-I domain in SEQ ID NO: 2 can be replaced with the corresponding helical I-I sequence from SEQ ID NO: 1, and the like. Domain sequences from reference CasX proteins, and their coordinates, are shown in Table 4.

Representative examples of chimeric CasX proteins of the disclosure include the CasX variants of SEQ ID NOS: 184-190, 197, 484, 9382-9542 and 9607-9609.

TABLE 3
Domain coordinates in Reference CasX proteins

| | Coordinates in | Coordinates in |
|---|---|---|
| Domain Name | SEQ ID NO: 1 | SEQ ID NO: 2 |
| OBD-I | 1-55 | 1-57 |
| helical I-I | 56-99 | 58-101 |
| NTSB | 100-190 | 102-191 |
| helical I_II | 191-331 | 192-332 |
| helical II | 332-508 | 333-500 |
| OBD_II | 509-659 | 501-646 |
| RuvC-I | 660-823 | 647-810 |
| TSL | 824-933 | 811-920 |
| RuvC-II | 934-986 | 921-978 |

Exemplary domain sequences are provided in Table 4 below.

TABLE 4
Exemplary Domain Sequences in Reference CasX proteins
Deltaproteobacter sp. (reference CasX of SEQ ID NO: 1)

| SEQ | | |
|---|---|---|
| ID | Domain | Sequence |
| 567 | OBD-I | EKRINKIRKKLSADNATKPVSRSGPMKTLLVRVMTDDLKKRLEKRRKKPEVMPQ |
| 568 | helical I-I | VISNNAANNLRMLLDDYTKMKEAILQVYWQEFKDDHVGLMCKFA |
| 569 | NTSB | QPASKKIDQNKLKPEMDEKGNLTTAGFACSQCGQPLFVYKLEQVSEKGKAYTNYFGRCNV |
| | | AEHEKLILLAQLKPEKDSDEAVTYSLGKFGQ |
| 570 | helical I-II | RALDFYSIHVTKESTHPVKPLAQIAGNRYASGPVGKALSDACMGTIASFLSKYQDIIIEH |
| | | QKVVKGNQKRLESLRELAGKENLEYPSVTLPPQPHTKEGVDAYNEVIARVRMWVNLNLWQ |
| | | KLKLSRDDAKPLLRLKGFPSF |
| 571 | helical II | PVVERRENEVDWWNTINEVKKLIDAKRDMGRVFWSGVTAEKRNTILEGYNYLPNENDHKK |
| | | REGSLENPKKPAKRQFGDLLLYLEKKYAGDWGKVFDEAWERIDKKIAGLTSHIEREEARN |
| | | AEDAQSKAVLTDWLRAKASFVLERLKEMDEKEFYACEIQLQKWYGDLRGNPFAVEAE |
| 572 | OBD-II | NRVVDISGFSIGSDGHSIQYRNLLAWKYLENGKREFYLLMNYGKKGRIRFTDGTDIKKSG |
| | | KWQGLLYGGGKAKVIDLTFDPDDEQLIILPLAFGTRQGREFIWNDLLSLETGLIKLANGR |
| | | VIEKTIYNKKIGRDEPALFVALTFERREVVD |
| 573 | RuvC-I | PSNIKPVNLIGVDRGENIPAVIALTDPEGCPLPEFKDSSGGPTDILRIGEGYKEKORAIQ |
| | | AAKEVEQRRAGGYSRKFASKSRNLADDMVRNSARDLFYHAVTHDAVLVFENLSRGFGROG |
| | | KRTFMTERQYTKMEDWLTAKLAYEGLTSKTYLSKTLAQYTSKTC |
| 574 | TSL | SNCGFTITTADYDGMLVRLKKTSDGWATTLNNKELKAEGQITYYNRYKRQTVEKELSAEL |
| | | DRLSEESGNNDISKWTKGRRDEALFLLKKRFSHRPVQEQFVCLDCGHEVH |
| 575 | RuvC-II | ADEQAALNIARSWLFLNSNSTEFKSYKSGKQPFVGAWQAFYKRRLKEVWKPNA |

Planctomycetes sp. (Reference CasX of SEQ ID NO: 2)

| SEQ | | |
|---|---|---|
| ID | Domain | Sequence |
| 576 | OBD-I | QEIKRINKIRRRLVKDSNTKKAGKTGPMKTLLVRVMTPDLRERLENLRKKPENIPQ |
| 577 | helical I-II | PISNTSRANLNKLLTDYTEMKKAILHVYWEEFQKDPVGLMSRVA |
| 578 | NTSB | QPAPKNIDORKLIPVKDGNERLTSSGFACSQCCQPLYVYKLEQVNDKGKPHTNYFGRCNV |
| | | SEHERLILLSPHKPEANDELVTYSLGKFGQ |
| 579 | helical I-II | RALDFYSIHVTRESNHPVKPLEQIGGNSCASGPVGKALSDACMGAVASFLTKYQDIILEH |
| | | QKVIKKNEKRLANLKDIASANGLAFPKITLPPQPHTKEGIEAYNNVVAQIVIWVNLNLWQ |
| | | KLKIGRDEAKPLQRLKGFPSF |
| 580 | helical II | PLVERQANEVDWWDMVCNVKKLINEKKEDGKVFWQNLAGYKRQEALLPYLSSEEDRKKGK |
| | | KFARYQFGDLLLHLEKKHGEDWGKVYDEAWERIDKKVEGLSKHIKLEEERRSEDAQSKAA |
| | | LTDWLRAKASFVIEGLKEADKDEFCRCELKLQKWYGDLRGKPFAIEAE |
| 581 | OBD-II | NSILDISGFSKQYNCAFIWQKDGVKKLNLYLIINYFKGGKLRFKKIKPEAFEANRFYTVI |
| | | NKKSGEIVPMEVNENFDDPNLIILPLAFGKROGREFIWNDLLSLETGSLK |
| | | LANGRVIEKTLYNRRTRQDEPALFVALTFERREVLD |
| 582 | RuvC-I | SSNIKPMNLIGIDRGENIPAVIALTDPEGCPLSRFKDSLGNPTHILRIGESYKEKORTIQ |
| | | AAKEVEQRRAGGYSRKYASKAKNLADDMVRNTARDLLYYAVTQDAMLIFENLSRGFGRQG |
| | | KRTEMAERQYTRMEDWLTAKLAYEGLPSKTYLSKTLAQYTSKTC |
| 583 | TSL | SNCGFTITSADYDRVLEKLKKTATGWMTTINGKELKVEGQITYYNRYKRONVVKDLSVEL |
| | | DRLSEESVNNDISSWTKGRSGEALSLLKKRFSHRPVQEKFVCLNCGFETH |
| 584 | RuvC-II | ADEQAALNIARSWLFLRSQEYKKYQTNKTTGNTDKRAFVETWQSFYRKKLKEVWKPAV |

d. Exemplary CasX Variants

In some embodiments, a CasX variant protein for use in the rAAV comprises a sequence set forth in Table 5 (SEQ ID NOS: 190, 197, 348, 351, 355, and 484). In some embodiments, a CasX variant protein for use in the rAAV comprises a sequence of SEQ ID NO: 197. In some embodiments, a CasX variant protein for use in the rAAV comprises a sequence of SEQ ID NO: 484. In other embodiments, a CasX variant protein comprises a sequence at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence selected from the group consisting of the sequences as set forth in SEQ ID NOS: 137-512, 9382-9542, and 9607-9609, wherein the variant retains the functional properties of the ability to form an RNP with a gRNA and to bind and cleave a target nucleic acid. In some embodiments, a CasX variant protein comprises a sequence selected from the group consisting of SEQ ID NOS: 9382-9542, and 9607-9609, or a sequence having at least about 70%, at least about 80%, at least about 90%, or at least about 95%, or at least about 95%, or at least about 96%, or at least about 97%, or at least about 98%, or at least about 99% sequence identity thereto, wherein the variant retains the functional properties of the ability to form an RNP with a gRNA and to bind and cleave a target nucleic acid. In some embodiments, a CasX variant protein comprises a sequence selected from the group consisting of SEQ ID NOS: 9382-9542, and 9607-9609. In other embodiments, a CasX variant comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical to a sequence selected from the group consisting of SEQ ID NOS: 197, 484, 9382-9542, and 9607-9609, and comprises a P at position 793 relative to SEQ ID NO: 2, wherein the CasX variant protein retains the functional properties of the ability to form an RNP with a gRNA and retains nuclease activity. In some embodiments, a CasX variant comprises a P at position 793 relative to SEQ TD NO: 2. In some embodiments, a CasX variant protein comprises a sequence of SEQ ID NO: 5. In some embodiments, a CasX variant protein consists of a sequence of SEQ ID NO: 5. As the results of the Examples demonstrate, despite changes in amino acid composition amongst the variants, the CasX variants retain the functional properties of the ability to form an RNP with a gRNA and retains nuclease activity, underscoring that the variants collectively have the ability to be utilized for a common use; the genetic editing of DNA.

TABLE 5
CasX Variant Sequences

| SEQ | | |
|---|---|---|
| ID NO | Variant | Description of Variant |
| 190 | 491 | QEIKRINKIRRRLVKDSNTKKAGKTGPMKTLLVRVMTPDLRERLENLRKKPENIPQPISNTSR |
| | | ANLNKLLTDYTEMKKAILHVYWEEFQKDPVGLMSRVAQPASKKIDQNKLKPEMDEKGNLTTAG |
| | | FACSQCGQPLFVYKLEQVSEKGKAYTNYFGRCNVAEHEKLILLAQLKPEKDSDEAVTYSLGKF |
| | | GQRALDFYSIHVTKESTHPVKPLAQIAGNRYASGPVGKALSDACMGTIASFLSKYQDIIIEHQ |
| | | KVVKGNQKRLESLRELAGKENLEYPSVTLPPQPHTKEGVDAYNEVIARVRMWVNLNLWQKLKL |
| | | SRDDAKPLLRLKGFPSFPLVERQANEVDWWDMVCNVKKLINEKKEDGKVFWQNLAGYKRQEAL |
| | | RPYLSSEEDRKKGKKFARYQLGDLLLHLEKKHGEDWGKVYDEAWERIDKKVEGLSKHIKLEEE |
| | | RRSEDAQSKAALTDWLRAKASFVIEGLKEADKDEFCRCELKLQKWYGDLRGKPFAIEAENSIL |
| | | DISGFSKQYNCAFIWQKDGVKKLNLYLIINYFKGGKLRFKKIKPEAFEANRFYTVINKKSGEI |
| | | VPMEVNENFDDPNLIILPLAFGKRQGREFIWNDLLSLETGSLKLANGRVIEKTLYNRRTRQDE |
| | | PALFVALTFERREVLDSSNIKPMNLIGVDRGENIPAVIALTDPEGCPLSRFKDSLGNPTHILR |
| | | IGESYKEKQRTIQAKKEVEQRRAGGYSRKYASKAKNLADDMVRNTARDLLYYAVTQDAMLIFE |
| | | NLSRGFGRQGKRTEMAERQYTRMEDWLTAKLAYEGLSKTYLSKTLAQYTSKTCSNCGFTITSA |
| | | DYDRVLEKLKKTATGWMTTINGKELKVEGQITYYNRYKRQNVVKDLSVELDRLSEESVNNDIS |
| | | SWTKGRSGEALSLLKKRFSHRPVQEKFVCLNCGFETHADEQAALNIARSWLFLRSQEYKKYQT |
| | | NKTTGNTDKRAFVETWQSFYRKKLKEVWKPAV |
| 197 | 515 | QEIKRINKIRRRLVKDSNTKKAGKTGPMKTLLVRVMTPDLRERLENLRKKPENIPQPISNTSR |
| | | ANLNKLLTDYTEMKKAILHVYWEEFQKDPVGLMSRVAQPASKKIDQNKLKPEMDEKGNLTTAG |
| | | FACSQCGQPLFVYKLEQVSEKGKAYTNYFGRCNVAEHEKLILLAQLKPEKDSDEAVTYSLGKF |
| | | GQRALDFYSIHVTKESTHPVKPLAQIAGNRYASGPVGKALSDACMGTIASFLSKYQDIIIEHQ |
| | | KVVKGNQKRLESLRELAGKENLEYPSVTLPPQPHTKEGVDAYNEVIARVRMWVNLNLWQKLKL |
| | | SRDDAKPLLRLKGFPSFPLVERQANEVDWWDMVCNVKKLINEKKEDGKVFWQNLAGYKRQEAL |
| | | RPYLSSEEDRKKGKKFARYQLGDLLLHLEKKHGEDWGKVYDEAWERIDKKVEGLSKHIKLEEE |
| | | RRSEDAQSKAALTDWLRAKASFVIEGLKEADKDEFCRCELKLQKWYGDLRGKPFAIEAENSIL |
| | | DISGFSKQYNCAFIWQKDGVKKLNLYLIINYFKGGKLRFKKIKPEAFEANRFYTVINKKSGEI |
| | | VPMEVNENFDDPNLIILPLAFGKRQGREFIWNDLLSLETGSLKLANGRVIEKTLYNRRTRQDE |
| | | PALFVALTFERREVLDSSNIKPMNLIGVDRGENIPAVIALTDPEGCPLSRFKDSLGNPTHILR |
| | | IGESYKEKQRTIQAKKEVEQRRAGGYSRKYASKAKNLADDMVRNTARDLLYYAVTQDAMLIFE |
| | | NLSRGFGRQGKRTFMAERQYTRMEDWLTAKLAYEGLPSKTYLSKTLAQYTSKTCSNCGFTITS |
| | | ADYDRVLEKLKKTATGWMTTINGKELKVEGQITYYNRYKRQNVVKDLSVELDRLSEESVNNDI |
| | | SSWTKGRSGEALSLLKKRFSHRPVQEKFVCLNCGFETHADEQAALNIARSWLELRSQEYKKYQ |
| | | TNKTTGNTDKRAFVETWQSFYRKKLKEVWKPAV |
| 348 | 668 | QEIKRINKIRRRLVKDSNTKKAGKTRGPMKTLLVRVMTPDLRERLENLRKKPENIPQPISNTS |
| | | RANLNKLLTDYTEMKKAILHVYWEEFQKDPVGLMSRVAQPASKKIDQNKLKPEMDEKGNLTTA |
| | | GFACSQCGQPLFVYKLEQVSEKGKAYTNYFGRCNVAEHEKLILLAQLKPEKDSDEAVTYSLGK |
| | | FGQRALDFYSIHVTKESTHPVKPLAQIAGNRYASSPVGKALSDACMGTIASFLSKYQDIIIEH |
| | | QKVVKGNQKRLESLRELAGKENLEYPSVTLPPQPHTKEGVDAYNEVIARVRMWVNLNLWQKLK |
| | | LSRDDAKPLLRLKGFPSFPLVERQANEVDWWDMVCNVKKLINEKKEDGKVFWQNLAGYKRQEA |
| | | LRPYLSSEEDRKKGKKFARYQLGDLLLHLEKKHGEDWGKVYDEAWERIDKKVEGLSKHIKLEE |
| | | ERRSEDAQSKAALTDWLRAKASFVIEGLKEADKDEFCRCELKLQKWYGDLRGKPFAIEAENSI |
| | | LDISGFSKQYNCAFIWQKDGVKKLNLYLIINYFKGGKLRFKKIKPEAFEANRFYTVINKKSGE |
| | | IVPMEVNENFDDPNLIILPLAFGKRQGREFIWNDLLSLETGSLKLANGRVIEKTLYNRRTRQD |
| | | EPALFVALTFERREVLDSSNIKPMNLIGVDRGENIPAVIALTDPEGCPLSRFKDSLGNPTHIL |
| | | RIGESYKEKQRTIQAKKEVEQRRAGGYSRKYASKAKNLADDMVRNTARDLLYYAVTQDAMLIF |
| | | ENLSRGFGRQGKRTFMAERQYTRMEDWLTAKLAYEGLPSKTYLSKTLAQYTSKTCSNCGFTIT |
| | | SADYDRVLEKLKKTATGWMTTINGKELKVEGQITYYNRYKRQNVVKDLSVELDRLSEESVNND |
| | | ISSWTKGRSGEALSLLKKRFSHRPVQEKFVCLNCGFETHADEQAALNIARSWLFLRSQEYKKY |
| | | QTNKTTGNTDKRAFVETWQSFYRKKLKEVWKPAV |
| 351 | 672 | QEIKRINKIRRRLVKDSNTKKAGKTGPMKTLLVRVMTPDLRERLENLRKKPENIPQPISNTSR |
| | | ANLNKLLTDYTEMKKAILHVYWEEFQKDPVGLMSRVAQPASKKIDQNKLKPEMDEKGNLTTAG |
| | | FACSQCGQPLFVYKLEQVSEKGKAYTNYFGRCNVAEHEKLIKLAQLKPEKDSDEAVTYSLGKF |
| | | GQRALDFYSIHVTKESTHPVKPLAQIAGNRYASSPVGKALSDACMGTIASFLSKYQDIIIEHQ |
| | | KVVKGNQKRLESLRELAGKENLEYPSVTLPPQPHTKEGVDAYNEVIARVRMWVNLNLWQKLKL |
| | | SRDDAKPLLRLKGFPSFPLVERQANEVDWWDMVCNVKKLINEKKEDGKVFWQNLAGYKRQEAL |
| | | RPYLSSEEDRKKGKKFARYQLGDLLLHLEKKHGEDWGKVYDEAWERIDKKVEGLSKHIKLEEE |
| | | RRSEDAQSKAALTDWLRAKASFVIEGLKEADKDEFCRCELKLQKWYGDLRGKPFAIEAENSIL |
| | | DISGFSKQYNCAFIWQKDGVKKLNLYLIINYFKGGKLRFKKIKPEAFEANRFYTVINKKSGEI |
| | | VPMEVNENFDDPNLIILPLAFGKRQGREFIWNDLLSLETGSLKLANGRVIEKTLYNRRTRQDE |
| | | PALFVALTFERREVLDSSNIKPMNLIGVDRGENIPAVIALTDPEGCPLSRFKDSLGNPTHILR |
| | | IGESYKEKQRTIQAKKEVEQRRAGGYSRKYASKAKNLADDMVRNTARDLLYYAVTQDAMLIFE |
| | | NLSRGFGRQGKRTEMAERQYTRMEDWLTAKLAYEGLPSKTYLSKTLAQYTSKTCSNCGFTITS |
| | | ADYDRVLEKLKKTATGWMTTINGKELKVEGQITYYNRYKRQNVVKDLSVELDRLSEESVNNDI |
| | | SSWTKGRSGEALSLLKKRFSHRPVQEKFVCLNCGFETHADEQAALNIARSWLFLRSQEYKKYQ |
| | | TNKTTGNTDKRAFVETWQSFYRKKLKEVWKPAV |
| 355 | 676 | QEIKRINKIRRRLVKDSNTKKAGKTRGPMKTLLVRVMTPDLRERLENLRKKPENIPQPISNTS |
| | | RANLNKLLTDYTEMKKAILHVYWEEFQKDPVGLMSRVAQPASKKIDQNKLKPEMDEKGNLTTA |
| | | GFACSQCGQPLFVYKLEQVSEKGKAYTNYFGRCNVAEHEKLIKLAQLKPEKDSDEAVTYSLGK |
| | | FGQRALDFYSIHVTKESTHPVKPLAQIAGNRYASSPVGKALSDACMGTIASFLSKYQDIIIEH |
| | | QKVVKGNQKRLESLRELAGKENLEYPSVTLPPQPHTKEGVDAYNEVIARVRMWVNLNLWQKLK |
| | | LSRDDAKPLLRLKGFPSFPLVERQANEVDWWDMVCNVKKLINEKKEDGKVFWQNLAGYKRQEA |
| | | LRPYLSSEEDRKKGKKFARYQLGDLLLHLEKKHGEDWGKVYDEAWERIDKKVEGLSKHIKLEE |
| | | ERRSEDAQSKAALTDWLRAKASFVIEGLKEADKDEFCRCELKLQKWYGDLRGKPFAIEAENSI |
| | | LDISGFSKQYNCAFIWQKDGVKKLNLYLIINYFKGGKLRFKKIKPEAFEANRFYTVINKKSGE |
| | | IVPMEVNENFDDPNLIILPLAFGKRQGREFIWNDLLSLETGSLKLANGRVIEKTLYNRRTRQD |
| | | EPALFVALTFERREVLDSSNIKPMNLIGVDRGENIPAVIALTDPEGCPLSRFKDSLGNPTHIL |
| | | RIGESYKEKQRTIQAKKEVEQRRAGGYSRKYASKAKNLADDMVRNTARDLLYYAVTQDAMLIF |
| | | ENLSRGFGRQGKRTFMAERQYTRMEDWLTAKLAYEGLPSKTYLSKTLAQYTSKTCSNCGFTIT |
| | | SADYDRVLEKLKKTATGWMTTINGKELKVEGQITYYNRYKRQNVVKDLSVELDRLSEESVNND |
| | | ISSWTKGRSGEALSLLKKRFSHRPVQEKFVCLNCGFETHADEQAALNIARSWLFLRSQEYKKY |
| | | QTNKTTGNTDKRAFVETWQSFYRKKLKEVWKPAV |
| 484 | 812 | QEIKRINKIRRRLVKDSNTKKAGKTGPMKTLLVRVMTPDLRERLENLRKKPENIPQPISNTSR |
| | | ANLNKLLTDYTEMKKAILHVYWEEFQKDPVGLMSRVAQPASKKIDQNKLKPEMDEKGNLTTAG |
| | | FACSQCGQPLFVYKLEQVSEKGKAYTNYFGRCNVAEHEKLILLAQLKPEKDSDEAVTYSLGKF |
| | | GQRALDFYSIHVTKESTHPVKPLAQIAGNRYASGPVGKALSDACMGTIASFLSKYQDIIIEHQ |
| | | KVVKGNQKRLESLRELAGKENLEYPSVTLPPQPHTKEGVDAYNEVIARVRMWVNLNLWQKLKL |
| | | SRDDAKPLLRLKKFPSFPLVERQANEVDWWDMVCNVKKLINEKKEDGKVFWQNLAGYKRQEAL |
| | | RPYLSSEEDRKKGKKFARYQLGDLLLHLEKKHGEDWGKVYDEAWERIDKKVEGLSKHIKLEEE |
| | | RRSEDAQSKAALTDWLRAKASFVIEGLKEADKDEFCRCELKLQKWYGDLRGKPFAIEAENSIL |
| | | DISGFSKQYNCAFIWQKDGVKKLNLYLIINYFKGGKLRFKKIKPEAFEANRFYTVINKKSGEI |
| | | VPMEVNENFDDPNLIILPLAFGKRQGREFIWNDLLSLETGSLKLANGRVIEKTLYNRRTRQDE |
| | | PALFVALTFERREVLDSSNIKPMNLIGVDRGENIPAVIALTDPEGCPLSRFKDSLGNPTHILR |
| | | IGESYKEKQRTIQAKKEVEQRRAGGYSRKYASKAKNLADDMVRNTARDLLYYAVTQDAMLIFE |
| | | NLSRGFGRQGKRTEMAERQYTRMEDWLTAKLAYEGLPSKTYLSKTLAQYTSKTCSNCGFTITS |
| | | ADYDRVLEKLKKTATGWMTTINGKELKVEGQITYYNRYKRQNVVKDLSVELDRLSEESVNNDI |
| | | SSWTKGRSGEALSLLKKRFSHRPVQEKFVCLNCGFETHADEQAALNIARSWLFLRSQEYKKYQ |
| | | TNKTTGNTDKRAFVETWQSFYRKKLKEVWKPAV |

Further CasX variants contemplated for use in the vectors of the disclosure are described in International Publication Nos. WO2020247882 and WO2022120095, which are hereby incorporated by reference in their entirety.

e. CasX Variants Derived from Other CasX Variants

In further iterations of the generation of variant proteins, a variant protein can be utilized to generate additional CasX variants of the disclosure. For example, CasX 119 (SEQ ID NO: 124), CasX 491 (SEQ ID NO: 190), and CasX 515 (SEQ ID NO: 197) are exemplary variant proteins that are modified to generate additional CasX variants of the disclosure having improvements or additional properties relative to a reference CasX or CasX variants from which they were derived. CasX 119 contains a substitution of L379R, a substitution of A708K and a deletion of P at position 793 of SEQ ID NO: 2. CasX 491 contains an NTSB and Helical 1B domain swap from SEQ ID NO: 1. CasX 515 was derived from CasX 491 by insertion of P at position 793 (relative to SEQ ID NO: 2) and was used to create additional CasX variants. For example, CasX 668 has an insertion of R at position 26 and a substitution of G223S relative to CasX 515. CasX 672 has substitutions of L169K and G223S relative to CasX 515. CasX 676 has substitutions of L169K and G223S and an insertion of R at position 26 relative to CasX 515. For purposes of the disclosure, the sequences of the domains of CasX 515 are provided in Table 6 and include an OBD-I domain having the sequence of SEQ ID NO: 585, an OBD-II domain having the sequence of SEQ ID NO: 590, NTSB domain having the sequence of SEQ ID NO: 587, a helical I-I domain having the sequence of SEQ ID NO: 586, a helical I-II domain having the sequence of SEQ ID NO: 588, a helical II domain having the sequence of SEQ ID NO: 589, a RuvC-I domain having the sequence of SEQ ID NO: 591, a RuvC-II domain having the sequence of SEQ ID NO: 593, and a TSL domain having the sequence of SEQ ID NO: 592.

Mutations can be introduced in any one or combinations of domains of the CasX variant to result in a CasX variant. These alterations can be amino acid insertions, deletions, substitutions, or any combinations thereof. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

In some embodiments, a CasX variant comprises two mutations relative to the CasX protein from which it was derived. In some embodiments, a CasX variant comprises three mutations relative to the CasX protein from which it was derived. In some embodiments, a CasX variant comprises 2, 3, 4, 5, 6, 7, 8, 9, 10 or more mutations relative to the CasX protein from which it was derived. In some embodiments, the 2, 3, 4, 5, 6, 7, 8, 9, 10 or more mutations are made in locations of the CasX protein sequence separated from one another. In other embodiments, the 2, 3, 4, 5, 6, 7, 8, 9, 10 or more mutations can be made in adjacent amino acids in the CasX protein sequence. In some embodiments, a CasX variant comprises two or more mutations relative to two or more different CasX proteins from which they were derived. The methods utilized for the design and creation of the CasX variant are described below, including the methods of the Examples.

Suitable mutagenesis methods for generating CasX variant proteins of the disclosure may include, for example, random mutagenesis, site-directed mutagenesis, Markov Chain Monte Carlo (MCMC)-directed evolution, staggered extension PCR, gene shuffling, rational design, or domain swapping (described in PCT/US2021/061673 and WO2020247882A1, incorporated by reference herein). In some embodiments, the CasX variant are designed, for example by selecting multiple desired mutations in a CasX variant identified, for example, using the approaches described in the Examples. In certain embodiments, the activity of the CasX variant protein prior to mutagenesis is used as a benchmark against which the activity of one or more resulting CasX variant are compared, thereby measuring improvements in function of the CasX variant.

k. CasX Variants Derived from CasX 515 (SEQ ID NO: 197)

The present disclosure provides highly-modified CasX variant proteins having multiple mutations relative to CasX 515. The mutations can be in one or more domains of the parental CasX 515 from which it was derived. The CasX domains and their positions, relative to CasX 515 (SEQ ID NO: 197) are presented in Table 5.

TABLE 6
CasX 515 domain sequences

| Domain | SEQ ID NO | Amino Acid Sequence |
|---|---|---|
| OBD-I | 585 | QEIKRINKIRRRLVKDSNTKKAGKTGPMKTLLVRVMTPDLRERLENLRK |
| | | KPENIPQ |
| Helical I-I | 586 | PISNTSRANLNKLLTDYTEMKKAILHVYWEEFQKDPVGLMSRVA |
| NTSB | 587 | QPASKKIDQNKLKPEMDEKGNLTTAGFACSQCGQPLFVYKLEQVSEKGK |
| | | AYTNY FGRCNVAEHEKLILLAQLKPEKDSDEAVTYSLGKFGQ |
| Helical I-II | 588 | RALDFYSIHVTKESTHPVKPLAQIAGNRYASGPVGKALSDACMGTIASF |
| | | LSKYQDIIIEHQKVVKGNQKRLESLRELAGKENLEYPSVTLPPQPHTKE |
| | | GVDAYNEVIARVRMWVNLNLWQKLKLSRDDAKPLLRLKGFPSF |
| Helical II | 589 | PLVERQANEVDWWDMVCNVKKLINEKKEDGKVFWQNLAGYKRQEALRPY |
| | | LSSEEDRKKGKKFARYQLGDLLLHLEKKHGEDWGKVYDEAWERIDKKVE |
| | | GLSKHIKLEEERRSEDAQSKAALTDWLRAKASFVIEGLKEADKDEFCRC |
| | | ELKLQKWYGDLRGKPFAIEAE |
| OBD-II | 590 | NSILDISGFSKQYNCAFIWQKDGVKKLNLYLIINYFKGGKLRFKKIKPE |
| | | AFEANRFYTVINKKSGEIVPMEVNENEDDPNLIILPLAFGKROGREFIW |
| | | NDLLSLETGSLKLANGRVIEKTLYNRRTRQDEPALEVALTFERREVLD |
| RuvC-I | 591 | SSNIKPMNLIGVDRGENIPAVIALTDPEGCPLSREKDSLGNPTHILRIG |
| | | ESYKEKQRTIQAKKEVEQRRAGGYSRKYASKAKNLADDMVRNTARDLLY |
| | | YAVTQDAMLIFENLSRGFGRQGKRTEMAERQYTRMEDWLTAKLAYEGLP |
| | | SKTYLSKTLAQYTSKTC |
| TSL | 592 | SNCGFTITSADYDRVLEKLKKTATGWMTTINGKELKVEGQITYYNRYKR |
| | | QNVVKDLSVELDRLSEESVNNDISSWTKGRSGEALSLLKKRFSHRPVQE |
| | | KFVCLNCGFETH |
| RuvC-II | 593 | ADEQAALNIARSWLFLRSQEYKKYQTNKTTGNTDKRAFVETWQSFYRKK |
| | | LKEVWKPAV |

In some embodiments of the CasX variant described herein, the approach to design the CasX variant utilizes a directed evolution method adapted from a Markov Chain Monte Carlo (MCMC)-directed evolution simulation (Biswas N., et al. Coupled Markov Chain Monte Carlo for high-dimensional regression with Half-t priors. arViV: 2012.04798v2 (2021)), as described in the Examples.

In further iterations of the generation of the CasX variant proteins, CasX 515 protein can be mutagenized to generate sequences resulting in amino acid substitutions, deletions, or insertions at one or more positions in one or more domains of the parental CasX 515 protein that are screened to identity CasX variants having improved or enhanced characteristics. Exemplary methods used to generate and evaluate CasX variants derived from the CasX 515 protein are described in the Examples. In some embodiments, the resulting mutagenized sequences are screened to identify those having enhanced nuclease activity. In other embodiments, the mutagenized sequences are screened to identify those having enhanced editing specificity and reduced off-target editing. In other embodiments, the mutagenized sequences are screened to identify those having enhanced PAM utilization; i.e., the ability to utilize non-canonical PAM sequences. In still other embodiments, the mutagenized sequences are screened to identify those having improved properties of any two or three of the foregoing categories; i.e., increased nuclease activity, increased specificity (reduced off-target editing), and enhanced PAM utilization. In other embodiments, libraries of sequence variants having one, two, three or more mutations at select positions relative to a parental CasX protein can be generated and screened in assays such as an E. coli CcdB toxin assay or a multiplexed pooled approach using a PASS assay to identify those CasX variants that had improved nuclease activity, improved specificity, and/or increased PAM utilization compared to the cleavage of the E. coli nucleic acid compared to the parental CasX 515 protein, as described in the Examples. In addition, the CasX variant can be screened for increased percentage of a eukaryotic genome that can be efficiently edited, improved ability to form cleavage-competent RNP with an gRNA, and improved stability of an RNP complex. In some embodiments, the improved characteristic compared to the parental CasX 515 is at least about 0.1-fold improved, at least about 0.5-fold improved, at least about 1-fold improved, at least about 1-fold improved, at least about 1-fold improved, at least about 1.5-fold improved, at least about 2-fold improved, at least about 3-fold improved, at least about 4-fold improved, at least about 5-fold improved, at least about 6-fold improved, at least about 7-fold improved, at least about 8-fold improved, at least about 9-fold improved, at least about 10-fold improved, or any integer in between the foregoing. In some embodiments, the characteristics are assayed in an in vitro assay.

In some embodiments, the disclosure provides CasX variants derived from CasX 515 (SEQ ID NO: 197) comprising two or more modifications; an insertion, a deletion, or a substitution of amino acid(s) in one or more domains (see Table 6 for CasX 515 domain sequences). In some embodiments, the disclosure provides CasX variant proteins comprising a pair of mutations relative to CasX 515 (SEQ ID NO: 9590) as depicted in Table 71, or further variations thereof. In some embodiments, a CasX variant comprising two or more modifications comprises a sequence selected from the group consisting of SEQ ID NOS: 9382-9542, and 9607-9609, or a sequence having at least about 70%, at least about 80%, at least about 90%, or at least about 95%, or at least about 95%, or at least about 96%, or at least about 97%, or at least about 98%, or at least about 99% sequence identity thereto. In a particular approach, as detailed in Example 38, single mutations of CasX 515 (SEQ ID NO: 9590) that demonstrated enhanced activity and/or specificity, were selected based on locations deemed to be potentially complementary, and combined (i.e., having two or three mutations) to make CasX variants that were then screened for activity and specificity in in vitro assays. The positions of the mutations within domains of CasX are described in detail in Table 72 in the Examples, below.

In some embodiments, the CasX variant derived from CasX 515 for use in the rAAV comprises a pair of mutations selected from the group consisting of 4.I.G & 64.R.Q, 4.I.G & 169.L.K, 4.I.G & 169.L.Q, 4.I.G & 171.A.D, 4.I.G & 171.A.Y, 41G & 171.A.S, 41G & 224.G.T, 4.I.G & 304.M.T, 4.1G & 398.Y.T, 4.I.G & 826.V.M, 4.I.G & 887.T.D, 4.I.G & 891.S.Q, 5.-.G & 64.R.Q, 5.-.G & 169.L.K, 5.-.G & 169.L.Q, 5.-.G & 171.A.D, 5.-.G & 171.A.Y, 5.-.G & 171.A.S, 5.-.G & 224.G.T, 5.-.G & 304.M.T, 5.-.G & 398.Y.T, 5.-.G & 826.V.M, 5.-.G & 887.T.D, 5.-.G & 891.S.Q, 9.K.G & 64.R.Q, 9.K.G & 169.L.K, 9.K.G & 169.L.Q, 9.K.G & 171.A.D, 9.K.G & 171AY, 9.K.G & 171.A.S, 9.K.G & 224.G.T, 9.K.G & 304.M.T, 9.K.G & 398.Y.T, 9.K.G & 826.V.M, 9.K.G & 887.T.D, 9.K.G & 891.S.Q, 27.-.R & 64.R.Q, 27.-.R & 169.L.K, 27.-.R & 169.L.Q, 27.-.R & 171.A.D, 27.-.R & 171AY, 27.-.R & 171.A.S, 27.-.R & 224.G.T, 27.-.R & 304.M. T, 27.-.R & 398.Y. T, 27.-.R & 826.V.M, 27.-.R & 887.T.D, 27.-.R & 891.S.Q, 35.R.P & 64.R.Q, 35.R.P & 169.L.K, 35.R.P & 169.L.Q, 35.R.P & 171.AD,35RP&71 AY, 35.R.P & 171.A.S, 35.R.P & 224.G.T, 35.R.P & 304.M.T, 35.R.P & 398.Y.T, 35.R.P & 826.V.M, 35.R.P & 887.T.D, 35.R.P & 891.S.Q, 887.T.D & 891.S.Q, 64.R.Q & 169.L.K, 64.R.Q & 169.L.Q, 64.R.Q & 171.A.D, 64.R.Q & 171.AY, 64.R.Q & 171.A.S, 64.R.Q & 224.G.T, 64.R.Q & 304.M.T, 64.R.Q & 398.Y.T, 64.R.Q & 826.V.M, 64.R.Q & 887.T.D, 64.R.Q & 891.S.Q, 169.L.K & 171.A.D, 169.L.K & 171AY, 169.L.K & 171.A.S, 169.L.K & 224.G.T, 169.L.K & 304.M.T, 169.L.K & 398.Y.T, 169.L.K & 826.V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398.Y.T, 169.L.Q & 826.V.M, 169.L.Q & 887.T.D, 169.L.Q & 891.S.Q, 171.A.D & 224.G.T, 171.A.D & 304.M.T, 171.A.D & 398.Y.T, 171AD & 826.V.M, 171.A.D & 887.T.D, 171AD & 891.S.Q, 171.A.Y & 224.G.T, 171.A.Y & 304.M.T, 171.A.Y & 398.Y.T, 171.A.Y & 826.V.M, 171.A.Y & 887.T.D, 171.A.Y & 891.S.Q, 171.A.S & 224.G.T, 171.A.S & 304.M.T, 171.A.S & 398.Y.T, 171.A.S & 826.V.M, 171.A.S & 887.T.D, 171.A.S & 891.S.Q, 4.1.G& 35.R.P, 224.G.T & 304.M.T, 224.G.T & 398.Y.T, 224.G.T & 826.V.M, 224.G.T & 887.T.D, 224.G.T & 891.S.Q, 5.-.G & 35.R.P, 4.I.G & 27.-.R, 304.M.T & 398.Y.T, 304.M.T & 826.V.M, 304.M.T & 887.T.D, 304.M.T & 891.S.Q, 9.K.G & 35.R.P, 5.-.G & 27.-.R, 41G & 9.K.G, 398.Y.T & 826.V.M, 398.Y.T & 887.T.D, 398.Y.T & 891.S.Q, 27.-.R & 35.R.P, 9.K.G & 27.-.R, 5.-.G & 9.K.G, 41G & 5.-.G, 826.V.M & 887.T.D, 826.V.M & 891.S.Q, 5.K.G & 27.-.R, 5.K.G & 169.L.K, 5.K.G & 171.A.D, 5.K.G & 304.M.T, 5.K.G & 398.Y.T, 5.K.G & 891.S.Q, 6.-.G & 27.-.R, 6.-.G & 169.L.K, 6.-.G & 171.A.D, 6.-.G & 304.M.T, 6.-.G & 398.Y.T, 6.-.G & 891.S.Q, 304.M.W & 27.-.R, 304.M.W & 169.L.K, 304.M.W & 171.A.D, 304.M.W & 398.Y.T, 304.M.W & 891.S.Q, 481.E.D & 27.-.R, 481.E.D & 169.L.K, 481.E.D & 171.A.D, 481.E.D & 304.M.T, 481.E.D & 398.Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q, as provided in Table 22, wherein the position of the mutations is relative to the CasX sequence of SEQ ID NO: 9590. In some embodiments, the CasX variant comprises one or more mutations from Table 22, wherein the one or more mutations result in an improved characteristic when expressed from an rAAV in a target cell compared to unmodified CasX 515 (SEQ ID NO: 197). In some embodiments, the improved characteristics is determined in an in vitro assay comprising a target nucleic acid, with the CasX complexed with a gRNA having a targeting sequence complementary to the target nucleic acid, compared to the unmodified parental CasX 515 under comparable conditions. In some embodiments, the improved characteristic is decreased off-target editing (or increased editing specificity), e.g., as shown in Table 76. In some embodiments, the improved characteristic is increased on-target editing, e.g., as shown in Table 75. In some embodiments, the improved characteristic is increased specificity ratio, e.g., as shown in Table 77.

In some embodiments, the CasX variant for use in an rAAV comprises three mutations in the sequence of CasX 515 (SEQ ID NO: 9590), wherein the three mutations are selected from the group consisting of 27.-.R, 169.L.K, and 329.G.K; 27.-.R, 171.A.D, and 224.G.T; and 35.R.P, 171.A.Y, and 304.M.T, wherein the mutations result in an improved characteristic compared to unmodified CasX 515.

In some embodiments, a CasX variant for use in an rAAV is selected from the group consisting of SEQ ID NOS: 9385, 9391, 9393, 9401, 9409, 9417, 9419, 9423, 9429, 9443, 9444, 9447, 9449, 9450, 9452, 9453, 9455, 9456, 9458, 9462, 9466, 9469, 9470, 9472, 9478, 9483, 9485, 9491, 9495, 9499, 9501, 9512, 9513, 9517, 9519, 9521, 9536, 9542, 9607, and 9609, wherein the CasX variant exhibits improved editing activity of a target nucleic acid compared to the unmodified parental CasX 515. In some embodiments, the improved characteristics is determined in an in vitro assay, complexed with a gRNA having a targeting sequence complementary to the target nucleic acid, compared to the unmodified parental CasX 515 and assayed under comparable conditions.

In some embodiments, a CasX variant for use in an rAAV is selected from the group consisting of SEQ ID NOS: 9385, 9386, 9388, 9390, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9486, 9487, 9488, 9492, 9493, 9496, 9509, 9512, 9516, 9517, 9519, 9521, 9522, 9529, 9536, 9542, 9608, and 9609, wherein the CasX variant exhibits improved editing specificity of a target nucleic acid compared to the unmodified parental CasX 515, In some embodiments, the improved characteristics is determined in an in vitro assay, complexed with a gRNA having a targeting sequence complementary to the target nucleic acid, compared to the unmodified parental CasX 515 and assayed under comparable conditions.

In some embodiments, a CasX variant for use in an rAAV is selected from the group consisting of SEQ ID NOS: 9385, 9409, 9417, 9443, 9444, 9447, 9450, 9452, 9455, 9466, 9469, 9470, 9472, 9478, 9512, 9513, 9517, 9519, 9521, 9536, 9542, and 9609, wherein the CasX variant exhibits improved editing activity and specificity of a target nucleic acid compared to the unmodified parental CasX 515. In some embodiments, the improved characteristics is determined in an in vitro assay, complexed with a gRNA having a targeting sequence complementary to the target nucleic acid, compared to the unmodified parental CasX 515 and assayed under comparable conditions.

In some embodiments, a CasX variant for use in an rAAV is selected from the group consisting of SEQ ID NOS: 9385, 9386, 9388, 9390, 9393, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9483, 9486, 9488, 9491, 9492, 9493, 9495, 9496, 9509, 9512, 9513, 9516, 9517, 9519, 9521, 9522, 9529, 9536, 9542, 9608, and 9609, wherein the CasX variant exhibits improved specificity ratio compared to the unmodified parental CasX 515. In some embodiments, the improved characteristics is determined in an in vitro assay, complexed with a gRNA having a targeting sequence complementary to the target nucleic acid, compared to the unmodified parental CasX 515 and assayed under comparable conditions.

In some embodiments, a CasX variant for use in an rAAV is selected from the group consisting of SEQ ID NOS: 9385, 9393, 9409, 9417, 9443, 9444, 9447, 9450, 9452, 9455, 9466, 9469, 9470, 9472, 9478, 9483, 9491, 9495, 9512, 9513, 9517, 9519, 9521, 9536, 9542, and 9609, wherein the CasX variant exhibits improved editing activity and improved specificity ratio compared to the unmodified parental CasX 515. In some embodiments, the improved characteristics is determined in an in vitro assay, complexed with a gRNA having a targeting sequence complementary to the target nucleic acid, compared to the unmodified parental CasX 515 and assayed under comparable conditions.

In some embodiments, the foregoing characteristics of the CasX variants are improved be at least about 0.1-fold, at least about 0.5-fold, at least about 1-fold, at least about 2-fold, at least about 4-fold, at least about 5-fold, at least about 6-fold, at least about 7-fold, at least about 8-fold, at least about 9-fold, or at least about 10-fold improved compared to the unmodified parental CasX 515.

l. CasX Fusion Proteins

Also contemplated within the scope of the disclosure are CasX variant proteins comprising a heterologous protein fused to the CasX. This includes CasX variants comprising N-terminal or C-terminal fusions of the CasX to a heterologous protein or domain thereof. In some embodiments, the CasX variant protein is fused to one or more proteins or domains thereof that has a different activity of interest, resulting in a fusion protein. For example, in some embodiments, the CasX variant protein is fused to a protein (or domain thereof) that inhibits transcription, modifies a target nucleic acid, or modifies a polypeptide associated with a nucleic acid (e.g., histone modification).

A variety of heterologous polypeptides are suitable for inclusion in a CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the fusion partner to a CasX variant has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., Hhal DNA m5c-methyltransferase (M.Hhal), DNA methyltransferase 1 (DNMT1), DNA methyltransferase 3 alpha (DNMT3A) and subdomains such as DNMT3A catalytic domain and ATRX-DNMT3-DNMT3L domain (ADD), DNMT3L interaction domain (DNMT3L), DNA methyltransferase 3 beta (DNMT3B), METI, ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat apolipoprotein B mRNA editing enzyme, catalytic polypeptide 1 {APOBEC1}), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a heterologous polypeptide (a fusion partner) for use with a CasX variant provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a CasX variant protein for use in the rAAV includes (is fused to) a nuclear localization signal (NLS) for targeting the CasX/gRNA to the nucleus of the cell. In some cases, a CasX variant protein is fused to 2 or more, 3 or more, 4 or more, or 5 or more 6 or more, 7 or more, 8 or more NLSs. In some embodiments, an NLS for incorporation into an rAAV of the disclosure comprises a sequence selected from the group consisting of SEQ ID NOS: 3411-3486, 3939-3971, 4065-4111. Non-limiting examples of NLSs suitable for use with a CasX variant include sequences having at least about 80%, at least about 90%, or at least about 95% identity or are identical to sequences derived from: the NLS of the SV40 virus large T-antigen, having the amino acid sequence PKKKRKV (SEQ ID NO: 3411); the NLS from nucleoplasmin (e.g., the nucleoplasmin bipartite NLS with the sequence KRPAATKKAGQAKKKK (SEQ ID NO: 3418); the c-myc NLS having the amino acid sequence PAAKRVKLD (SEQ ID NO: 3420) or RQRRNELKRSP (SEQ ID NO: 4065); the hRNPAI M9 NLS having the sequence NQSSNFGPMKGGNFGGRSSGPYGGGGQYFAKPRNQGGY (SEQ ID NO: 4066); the sequence RMRIZFKNKGKDTAELRRRRVEVSVELRKAKKDEQILKRRNV (SEQ ID NO: 4067) of the IBB domain from importin-alpha; the sequences VSRKRPRP (SEQ ID NO: 4068) and PPKKARED (SEQ ID NO: 4069) of the myoma T protein; the sequence PQPKKKPL (SEQ ID NO: 4070) of human p53; the sequence SALIKKKKKMAP (SEQ ID NO: 4071) of mouse c-abl IV; the sequences DRLRR (SEQ ID NO: 4072) and PKQKKRK (SEQ ID NO: 4073) of the influenza virus NS1; the sequence RKLKKKIKKL (SEQ ID NO: 4074) of the Hepatitis virus delta antigen; the sequence REKKKFLKRR (SEQ ID NO: 4075) of the mouse Mxl protein; the sequence KRKGDEVDGVDEVAKKKSKK (SEQ ID NO: 4076) of the human poly(ADP-ribose) polymerase; the sequence RKCLQAGMNLEARKTKK (SEQ ID NO: 4077) of the steroid hormone receptors (human) glucocorticoid; the sequence PRPRKIPR (SEQ ID NO: 4078) of Borna disease virus P protein (BDV-P1); the sequence PPRKKRTVV (SEQ ID NO: 4079) of hepatitis C virus nonstructural protein (HCV-NS5A); the sequence NLSKKKKRKREK (SEQ ID NO: 4080) of LEF1; the sequence RRPSRPFRKP (SEQ ID NO: 4081) of ORF57 simirae; the sequence KRPRSPSS (SEQ ID NO: 4082) of EBV LANA; the sequence KRGINDRNFWRGENERKTR (SEQ ID NO: 4083) of Influenza A protein; the sequence PRPPKMARYDN (SEQ ID NO: 4084) of human RNA helicase A (RHA); the sequence KRSFSKAF (SEQ ID NO: 4085) of nucleolar RNA helicase II; the sequence KLKIKRPVK (SEQ ID NO: 4086) of TUS-protein; the sequence PKKKRKVPPPPAAKRVKLD (SEQ ID NO: 4087) associated with importin-alpha; the sequence PKTRRRPRRSQRKRPPT (SEQ ID NO: 4088) from the Rex protein in HTLV-1; the sequence MSRRRKANPTKLSENAKKLAKEVEN (SEQ ID NO: 4089) from the EGL-13 protein of Caenorhabditis elegans; and the sequences KTRRRPRRSQRKRPPT (SEQ ID NO: 4090), RRKKRRPRRKKRR (SEQ ID NO: 4091), PKKKSRKPKKKSRK (SEQ ID NO: 4092), HKKKHPDASVNFSEFSK (SEQ ID NO: 4093), QRPGPYDRPQRPGPYDRP (SEQ ID NO: 4094), LSPSLSPLLSPSLSPL (SEQ ID NO: 4095), RGKGGKGLGKGGAKRHRK (SEQ ID NO: 14096), PKRGRGRPKRGRGR (SEQ ID NO: 4097), PKKKRKVPPPPAAKRVKLD (SEQ ID NO: 4098) and PKKKRKVPPPPKKKRKV (SEQ ID NO: 4099), PAKRARRGYKC (SEQ ID NO: 3425), KLGPRKATGRW (SEQ ID NO: 4100), PRRKREE (SEQ ID NO: 4101), PYRGRKE (SEQ ID NO: 4102), PLRKRPRR (SEQ ID NO: 4103), PLRKRPRRGSPLRKRPRR (SEQ ID NO: 4104), PAAKRVKLDGGKRTADGSEFESPKKKRKV (SEQ ID NO: 4105), PAAKRVKLDGGKRTADGSEFESPKKKRKVGIHGVPAA (SEQ ID NO: 4106), PAAKRVKLDGGKRTADGSEFESPKKKRKVAEAAAKEAAAKEAAAKA (SEQ ID NO: 4107), PAAKRVKLDGGKRTADGSEFESPKKKRKVPG (SEQ ID NO: 4108), KRKGSPERGERKRHW (SEQ ID NO: 4109), KRTADSQHSTPPKTKRKVEFEPKKKRKV (SEQ ID NO: 4110), and PKKKRKVGGSKRTADSQHSTPPKTKRKVEFEPKKKRKV (SEQ ID NO: 4111). Additional NLS for incorporation in the rAAV of the disclosure are provided in Tables 20 and 21, indicating NLS for linking to the N- or C-terminus of the CasX. In some embodiments, the one or more NLS are linked to the CasX or to an adjacent NLS by a linker peptide wherein the linker peptide is selected from the group consisting of RS, (G)n (SEQ ID NO: 26), (GS)n (SEQ ID NO: 27), (GSGGS)n (SEQ ID NO: 20), (GGSGGS)n (SEQ ID NO: 21), (GGGS)n (SEQ ID NO: 22), GGSG (SEQ ID NO: 23), GGSGG (SEQ ID NO: 24), GSGSG (SEQ ID NO: 25), GSGGG (SEQ ID NO: 28), GGGSG (SEQ ID NO: 45), GSSSG (SEQ ID NO: 46), GPGP (SEQ ID NO: 29), GGP, PPP, PPAPPA (SEQ ID NO: 30), PPPG (SEQ ID NO: 47), PPPGPPP (SEQ ID NO: 31), PPP(GGGS)n (SEQ ID NO: 44), (GGGS)nPPP (SEQ ID NO: 32), AEAAAKEAAAKEAAAKA (SEQ ID NO: 4112), and TPPKTKRKVEFE (SEQ ID NO: 4113), wherein n is 1 to 5. In some embodiments, the rAAV constructs of the disclosure comprise polynucleic acids encoding the NLS and linker peptides of any of the foregoing embodiments of the paragraph, as well as the NLS of Tables 20 and 21, and can be, in some cases, configured in relation to the other components of the transgene constructs as depicted in any one of FIG. 1, 25, 38-40, 47, or 75.

In general, NLS (or multiple NLSs) are of sufficient strength to drive accumulation of a CasX variant fusion protein in the nucleus of a eukaryotic cell. Detection of accumulation in the nucleus may be performed by any suitable technique. For example, a detectable marker may be fused to a CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some embodiments, a CasX variant fusion protein can include a CasX protein that is linked to an internally inserted heterologous amino acid or heterologous polypeptide (a heterologous amino acid sequence) via a linker polypeptide (e.g., one or more linker polypeptides). In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)n, glycine-serine polymers, glycine-alanine polymers, alanine-serine polymers, glycine-proline polymers, proline polymers and proline-alanine polymers. Example linkers can comprise amino acid sequences including, but not limited to (G)n (SEQ ID NO: 26), (GS)n (SEQ ID NO: 27), (GSGGS)n (SEQ ID NO: 20), (GGSGGS)n (SEQ ID NO: 21), (GGGS)n (SEQ ID NO: 22), GGSG (SEQ ID NO: 23), GGSGG (SEQ ID NO: 24), GSGSG (SEQ ID NO: 25), GSGGG (SEQ ID NO: 28), GGGSG (SEQ ID NO: 45), GSSSG (SEQ ID NO: 46), GPGP (SEQ ID NO: 29), GGP, PPP, PPAPPA (SEQ ID NO: 30), PPPG (SEQ ID NO: 47), PPPGPPP (SEQ ID NO: 31), PPP(GGGS)n (SEQ ID NO: 44), (GGGS)nPPP (SEQ ID NO: 32), AEAAAKEAAAKEAAAKA (SEQ ID NO:4112), and TPPKTKRKVEFE (SEQ ID NO: 4113), where n is 1 to 5, where n is 1 to 5. The ordinarily skilled artisan will recognize that design of a peptide conjugated to any elements described above can include linkers that are all or partially flexible, such that the linker can include a flexible linker as well as one or more portions that confer less flexible structure.

V. rAAV and Methods for Modification of Target Nucleic Acids

The rAAV provided herein are useful for various applications, including as therapeutics, diagnostics, and for research. To effect the methods of the disclosure for gene editing, provided herein are programmable rAAV to modify the target nucleic acid in eukaryotic cells; either in vitro, ex vivo, or in vivo in a subject. Generally, any portion of a gene can be targeted using the programmable systems and methods provided herein. In some embodiments of the rAAV vector, the CRISPR nuclease is a Class 2, Type V nuclease. In some embodiments, the disclosure provides a Class 2, Type V nuclease selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and Cas(D. In some embodiments, the disclosure provides vectors encoding a CasX variant protein and one or more guide nucleic acid (gRNA) variants as gene editing pairs. The programmable nature of the CasX and gRNA components of the rAAV provided herein allows for the precise targeting to achieve the desired effect (nicking, cleaving, etc.) at one or more regions of predetermined interest in the target nucleic acid sequence. In some embodiments, the rAAV provided herein comprise sequences encoding a CasX variant protein and a first, and optionally a second gRNA wherein the targeting sequence of the gRNA is complementary to, and therefore is capable of hybridizing with, a target nucleic acid sequence. In some cases, the rAAV further comprises a donor template nucleic acid.

In some embodiments of the disclosure, provided herein are methods of modifying a target nucleic acid sequence. In some embodiments, the methods comprise contacting a cell comprising the target nucleic acid sequence with an rAAV encoding a CasX protein of the disclosure and a gRNA of the disclosure comprising a targeting sequence, wherein the targeting sequence of the gRNA has a sequence complementary to and that can hybridize with the sequence of the target nucleic acid. Upon hybridization with the target nucleic acid by the CasX and the gRNA, the CasX introduces one or more single-strand breaks or double-strand breaks within or near the target nucleic acid, which may include sequences that contain regulatory elements or non-coding regions of the gene, that results in a permanent indel (deletion or insertion) or mutation in the target nucleic acid, as described herein, with a corresponding modulation of expression or alteration in the function of the gene product, thereby creating an edited cell. In some embodiments of the method, the modification comprises introducing an in-frame mutation in the target nucleic acid. In some embodiments of the method, the modification comprises introducing a frame-shifting mutation in the target nucleic acid. In some embodiments of the method, the modification comprises introducing a premature stop codon in the coding sequence in the target nucleic acid. In some embodiments of the method, the modification results in expression of a non-functional protein in the modified cells of the population. In some embodiments of the method, the modification results in the correction of a mutation to wild-type or results in the ability of the cell to express a functional gene product.

In some embodiments of the method of modifying a target nucleic acid sequence, the method comprises contacting a cell with an rAAV comprising an encoded CasX protein wherein the CasX is an encoded CasX variant having a sequence of any one of SEQ ID NOS: 137-512, 9382-9542, and 9607-9609, or a sequence having at least about 80%, at least about 90%, or at least about 95%, or at least about 96%, or at least about 97%, or at least about 98%, or at least about 99% sequence identity thereto, and comprises a gRNA scaffold having a sequence of SEQ ID NOS: 2238-2400, 9257-9289 and 9588, or a sequence having at least about 80%, at least about 90%, or at least about 95%, or at least about 96%, or at least about 97%, or at least about 98%, or at least about 99% sequence identity thereto, and comprises a targeting sequence complementary to the target nucleic acid to be modified, wherein the expressed CasX and gRNA retain the ability to form an RNP complex and to bind and cleave the target nucleic acid. In some embodiments of the method of modifying a target nucleic acid sequence, the method comprises contacting a cell with an rAAV comprising an encoded CasX variant having a sequence selected from the group consisting of SEQ ID NOS: 190, 197, 278, 352, 355, 359, and 484, or a sequence having at least about 80%, at least about 90%, or at least about 95%, or at least about 96%, or at least about 97%, or at least about 98%, or at least about 99% sequence identity thereto, and comprises a gRNA scaffold having a sequence of SEQ ID NOS: 2292, or a sequence having at least about 80%, at least about 90%, or at least about 95%, or at least about 96%, or at least about 97%, or at least about 98%, or at least about 99% sequence identity thereto, and comprises a targeting sequence complementary to the target nucleic acid to be modified, wherein the expressed CasX and gRNA retain the ability to form an RNP complex and to bind and cleave the target nucleic acid. In some embodiments of the method of modifying a target nucleic acid sequence, the method comprises contacting a cell with an rAAV comprising an encoded CasX variant having a sequence selected from the group consisting of SEQ ID NOS: 190, 197, 278, 352, 355, 359, and 484, or a sequence having at least about 80%, at least about 90%, or at least about 95%, or at least about 96%, or at least about 97%, or at least about 98%, or at least about 99% sequence identity thereto, and comprises a gRNA scaffold having a sequence of SEQ ID NOS: 9588, or a sequence having at least about 80%, at least about 90%, or at least about 95%, or at least about 96%, or at least about 97%, or at least about 98%, or at least about 99% sequence identity thereto, and comprises a targeting sequence complementary to the target nucleic acid to be modified, wherein the expressed CasX and gRNA retain the ability to form an RNP complex and to bind and cleave the target nucleic acid.

In other embodiments, the method comprises contacting a cell comprising the target nucleic acid sequence with an rAAV encoding a first and a second of gRNA targeted to different or overlapping portions of the target nucleic acid wherein the CasX protein introduces multiple breaks in the target nucleic acid that result in a permanent indel, mutation, or excision of the intervening sequence in the target nucleic acid, with a corresponding modulation of expression or alteration in the function of the gene product, thereby creating an edited cell. In some embodiments of the method, the gRNA scaffold of the first and the second comprises a sequence selected from the group consisting of SEQ ID NOS: 2238-2400, 9257-9289 and 9588. In some embodiments of the method, the gRNA scaffold of the first and the second comprises a sequence selected from the group consisting of SEQ ID NOS: 2238 and 2292.

In some embodiments of the method, the modification of the target nucleic acid results in reduced expression of a gene product of a gene comprising the target nucleic acid, wherein expression is reduced by at least about 10%, at least about 20%, at least about 30%, at least about 40%, at least about 50%, at least about 60%, at least about 70%, at least about 80%, or at least about 90% in comparison to a cell that has not been modified. In some embodiments of the method, the modification of the target nucleic acid results in correction of a mutation in the target nucleic acid such that a wild-type or a functional gene product can be express.

In some embodiments, the modifying of the target nucleic acid sequence is carried out ex vivo. In some embodiments, the modifying of the target nucleic acid sequence is carried out in vitro inside a cell. In some embodiments of the modification of the target nucleic acid sequence in a cell, the cell is a eukaryotic cell selected from the group consisting of a rodent cell, a mouse cell, a rat cell, a primate cell, a non-human primate cell, and a human cell. In particular embodiments, the eukaryotic cell is a human cell. In some embodiments, the modifying of the target nucleic acid sequence is carried out in vivo in a subject. In some embodiments, the subject is selected from the group consisting of mouse, rat, pig, non-human primate. In some embodiments, the subject is a human.

In some embodiments, the method of modifying a target nucleic acid sequence comprises contacting a target nucleic acid with an rAAV encoding a CasX protein and gRNA pair and further comprising a donor template. The donor template may be inserted into the target nucleic acid such that all, some or none of the gene product is expressed. Depending on whether the vector is used to knock-down/knock-out or to knock-in a protein-coding sequence, the donor template can be a short single-stranded or double-stranded oligonucleotide, or can be a long single-stranded or double-stranded oligonucleotide. For knock-down/knock-outs, the donor template sequence need not be identical to the genomic sequence that it replaces and may contain one or more single base changes, insertions, deletions, inversions or rearrangements with respect to the genomic sequence. Provided that there are arms with sufficient numbers of nucleotides having sufficient homology flanking the cleavage site(s) of the target nucleic acid sequence targeted by the CasX:gRNA (i.e., 5′ and 3′ to the cleavage site) to support homology-directed repair (“homologous arms”), use of such donor templates can result in a frame-shift or other mutation such that the gene product is not expressed or is expressed at a lower level. In some embodiments, the homologous arms comprise between 10 and 100 nucleotides. The upstream and downstream homology arm sequences share at least about 80%, 85%, 90%, 95%, or 100% homology with the nucleotide sequences within 1-50 bases flanking either side of the cleavage site where the CasX cleaves the target nucleic acid sequence, facilitating insertion of the donor template sequence by HDR. In some embodiments, the donor template sequence comprises a non-homologous or a heterologous sequence flanked by two homologous arms, such that homology-directed repair between the target DNA region and the two flanking arm sequences results in insertion of the non-homologous or heterologous sequence at the target region, resulting in the knock-down or knock-out of the target gene, with a resulting reduction or elimination of expression of the gene product. In such knock-down cases, expression of the gene product is reduced by at least about 10%, at least about 20%, at least about 30%, at least about 40%, at least about 50%, at least about 60%, at least about 70%, at least about 80%, or at least about 90% in comparison to target nucleic acid that has not been modified. In other cases, an exogenous donor template may comprise a corrective sequence to be integrated, and is flanked by an upstream homologous arm and a downstream homologous arm, each having homology to the target nucleic acid sequence that is introduced into a cell. Use of such donor templates can result in expression of functional protein or expression of physiologically normal levels of functional protein after gene editing. In other cases, an exogenous donor template, which may comprise a mutation, a heterologous sequence, or a corrective sequence, is inserted between the ends generated by CasX cleavage by homology-independent targeted integration (HITI) mechanisms. The exogenous sequence inserted by HITI can be any length, for example, a relatively short sequence of between 1 and 50 nucleotides in length, or a longer sequence of about 50-1000 nucleotides in length. The lack of homology can be, for example, having no more than 20-50% sequence identity and/or lacking in specific hybridization at low stringency. In other cases, the lack of homology can further include a criterion of having no more than 5, 6, 7, 8, or 9 bp identity.

Introducing recombinant rAAV into a target cell can be carried out in vivo, in vitro or ex vivo. Introducing recombinant rAAV comprising sequences encoding the transgene components (e.g., the CasX, gRNA, promoters and accessory components and, optionally, the donor template sequences) of the disclosure into cells under in vitro conditions can occur in any suitable culture media and under any suitable culture conditions that promote the survival of the cells and production of the CasX:gRNA. In some embodiments of the method, vectors may be provided directly to a target host cell. For example, cells may be contacted with vectors having nucleic acids encoding the CasX and gRNA of any of the embodiments described herein and, optionally, having a donor template sequence such that the vectors are taken up by the cells.

In some embodiments, the vector is administered in vivo to a subject at a therapeutically effective dose. In the foregoing, the subject is selected from the group consisting of mouse, rat, pig, non-human primate, and human. In particular embodiments, the subject is a human. In some embodiments of the methods, the vector is administered to a subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, at least about 1×10¹⁶ vg/kg. In other embodiments, the vector is administered to the subject at a dose of at least about 1×10⁵ vg/kg to at least about 1×10¹⁶ vg/kg, or at least about 1×10⁶ vg/kg to about 1×10¹⁵ vg/kg, or at least about 1×10⁷ vg/kg to about 1×10¹⁴ vg/kg, or at least about 1×10¹ vg/kg to about 1×10¹⁴ vg/kg.

The vector can be administered by a route of administration selected from the group consisting of subcutaneous, intradermal, intraneural, intranodal, intramedullary, intramuscular, intralumbar, intrathecal, subarachnoid, intraventricular, intracapsular, intravenous, intralymphatical, or intraperitoneal routes, wherein the administering method is injection, transfusion, or implantation.

VI. rAAV

In other embodiments, the present disclosure provides recombinant rAAV comprising polynucleotides encoding the CasX proteins, the gRNAs, and the regulatory and accessory elements described herein that are integrated into the rAAV transgene.

In some embodiments, the disclosure provides a recombinant adeno-associated virus (rAAV) comprising: a) an AAV capsid protein, and b) the transgene polynucleotide of any one of the embodiments described herein. In the foregoing embodiment, the polynucleotide can comprise sequences of components selected from: a first adeno-associated virus (AAV) inverted terminal repeat (ITR) sequence; a second AAV ITR sequence; a first promoter sequence operably linked to the CRISPR protein; a second promoter sequence operably linked to the gRNA; a sequence encoding a CRISPR protein; a sequence encoding at least a first guide RNA (gRNA); and one or more accessory element sequences (e.g., a 3′ UTR, a poly(A) signal sequence, an enhancer, an intron, a posttranscriptional regulatory element (PTREs), an NLS, a deaminases, a DNA glycosylase inhibitor, a factor that stimulates CRISPR-mediated homology-directed repair, an activator or repressor of transcription, a self-cleaving sequence, or a fusion domain. In some embodiments, the polynucleotide comprises one or more sequences selected from the group of sequences set forth in Tables 7-10, 12-17, 19, 23-43, 45-46, 50-55, 57-58, and 60-61, or a sequence having at least 85%, at least 90%, at least 95%, at least 95%, at least 96%, at least 97%, at least 98%, or at least 99% identity thereto. In another embodiment, the polynucleotide comprises a sequence selected from the group of sequences set forth in Tables 7-10, 12-17, 19, 23-43, 45-46, 50-55, 57-58, and 60-61. In some embodiments, the polynucleotide sequence differs from those set forth in Tables 7-10, 12-17, 19, 23-43, 45-46, 50-55, 57-58, and 60-61 only in the selection of the targeting sequences of the gRNA or gRNAs encoded by the polynucleotide, wherein the targeting sequence is a sequence having 15 to 20 nucleotides capable of hybridizing with the sequence of a target nucleic acid. In some embodiments, the present disclosure provides a transgene polynucleotide, wherein the polynucleotide has the configuration of a construct of any one of FIG. 1, 25, 28, 38-40, 47 or 75.

In some embodiments, the AAV capsid protein is derived from serotype AAV1, AAV2, AAV3, AAV4, AAV5, AAV6, AAV7, AAV8, AAV9, AAV10, AAV11, AAV12, AAV 9.45, AAV 9.61, AAV 44.9, AAV-Rh74, AAVRh10, MyoAAV 1Al, MyoAAV 1A2, or MyoAAV 2A. In some embodiments, the AAV capsid protein and the 5′ and 3′ ITR are derived from the same serotype of AAV. In other embodiments, the AAV capsid protein and the 5′ and 3′ ITR are derived from different serotypes of AAV. In a particular embodiment, the 5′ and 3′ ITR are derived from AAV1. In a particular embodiment, the ITRs are derived from serotype AAV2, including the 5′ ITR having sequence CCTGCAGGCAGCTGCGCGCTCGCTCGCTCACTGAGGCCGCCCGGGCGTCGGGCGAC CTTTGGTCGCCCGGCCTCAGTGAGCGAGCGAGCGCGCAGAGAGGGAGTGGCCAACT CCATCACTAGGGGTTCCT (SEQ ID NO: 3683) and the 3′ ITR having sequence AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGCTGCCTGCAGG (SEQ ID NO: 3701).

In some embodiments, the polynucleotides utilized in the rAAV comprise sequences encoding a CasX variant selected from the group consisting of SEQ ID NOS: 137-512, 9382-9542, and 9607-9609, or sequences having at least about 70%, at least about 80%, at least about 90%, at least about 95%, at least about 96%, at least about 97%, at least about 98%, or at least about 99% sequence identity thereto. In some embodiments, the polynucleotides utilized in the rAAV comprise sequences encoding the CasX variants selected from the group consisting of SEQ ID NOS: 190, 197, 348, 351, 355, or 484, or sequences having at least about 70%, at least about 80%, at least about 90%, at least about 95%, at least about 96%, at least about 97%, at least about 98%, or at least about 99% sequence identity thereto. In some embodiments, the polynucleotides utilized in the rAAV encode gRNA scaffold sequences selected from the group consisting of SEQ ID NOS: 2238-2400, 9257-9289 and 9588, or sequences having at least about 70%, at least about 80%, at least about 90%, at least about 95%, at least about 95%, at least about 96%, at least about 97%, at least about 98%, at least about 99% sequence identity thereto. In some embodiments, the polynucleotides utilized in the rAAV encode gRNA scaffold sequences selected from the group consisting of SEQ ID NOS: 2292 and 9588 as set forth in Table 2, or sequences having at least about 70%, at least about 80%, at least about 90%, at least about 95%, at least about 95%, at least about 96%, at least about 97%, at least about 98%, at least about 99% sequence identity thereto. In some embodiments, the gRNA comprises a targeting sequence having 15 to 20 nucleotides that is complementary to, and therefore hybridizes with, the target nucleic acid in a cell, and is linked to the 3′ end of the gRNA scaffold sequence. In one embodiment, the polynucleotide utilized in the rAAV transgene encodes CasX 515 (SEQ ID NO: 197), gRNA scaffold 235 (SEQ ID NO: 2292), and the gRNA comprises a targeting sequence having 15 to 20 nucleotides that is complementary to, and therefore hybridizes with, the target nucleic acid in a cell, and is linked to the 3′ end of the gRNA scaffold sequence. In another embodiment, the polynucleotide utilized in the rAAV transgene encodes CasX 515 (SEQ ID NO: 197), gRNA scaffold 316 (SEQ ID NO: 9588), and the gRNA comprises a targeting sequence having 15 to 20 nucleotides that is complementary to, and therefore hybridizes with, the target nucleic acid in a cell, and is linked to the 3′ end of the gRNA scaffold sequence.

In other embodiments, the disclosure provides an rAAV comprising a donor template nucleic acid, wherein the donor template comprises a nucleotide sequence having homology to a target nucleic acid sequence. In some embodiments, the donor template is intended for gene editing and comprises all or at least a portion of a target gene wherein upon insertion of the donor template, the gene is either knocked down, knocked out, or the mutation is corrected. In some embodiments, the donor template comprises a sequence that encodes at least a portion of a target nucleic acid exon. In other embodiments, the donor template has a sequence that encodes at least a portion of a target nucleic acid intron. In other embodiments, the donor template has a sequence that encodes at least a portion of a target nucleic acid intron-exon junction. In still other cases, the donor template sequence of the rAAV comprises one or more mutations relative to a target nucleic acid. In the foregoing embodiments, the donor template can range in size from 10-700 nucleotides. In some embodiments, the donor template is a single-stranded DNA template.

In other aspects, the disclosure relates to methods to produce polynucleotide sequences encoding the rAAV, as well as methods to express and recover the rAAV. In general, the methods include producing a polynucleotide sequence coding for the components of the expression cassette plus the flanking ITRs and incorporating the encoding gene into an expression vector appropriate for a host cell. For production of the rAAV, the methods include transforming an appropriate host cell with an expression vector comprising the encoding polynucleotide, together with and the Rep and Cap sequences provided in trans, and culturing the host cell under conditions causing or permitting the resulting rAAV to be produced, which are recovered by methods described herein or by standard purification methods known in the art. Rep and Cap can be provided to the packaging host cell as plasmids. Alternatively, the host cell genome may comprise stably integrated Rep and Cap genes. Suitable packaging cell lines are known to one of ordinary skill in the art. See for example, www.cellbiolabs.com/aav-expression-and-packaging. Methods of purifying rAAV produced by host cell lines will be known to one of ordinary skill in the art, and include, without limitation, affinity chromatography, gradient centrifugation, and ion exchange chromatography. Standard recombinant techniques in molecular biology are used, along with the methods of the Examples, to make the polynucleotides and rAAV of the present disclosure.

In accordance with the disclosure, nucleic acid sequences that encode the CasX variants or the gRNA described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the CasX variants or the gRNA that is used to transform a host cell for expression of the composition.

In some approaches, a construct is first prepared containing the DNA sequences encoding the components of the rAAV and transgene. Exemplary methods for the preparation of such constructs are described in the Examples. The construct is then used to create an expression vector suitable for transforming a host packaging cell, such as a eukaryotic host cell for the expression and recovery of the rAAV comprising the transgene. The eukaryotic host packaging cell can be selected from Baby Hamster Kidney fibroblast (BHK) cells, human embryonic kidney 293 (HEK293), human embryonic kidney 293T (HEK293T), NSO cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO) cells, or other eukaryotic cells known in the art suitable for the production of recombinant AAV. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles. Exemplary methods for the creation of expression vectors, the transformation of host cells and the expression and recovery of the nucleic acids and the rAAV are described in the Examples.

The gene encoding the rAAV can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., ITRs, CasX and gRNA, promoters and accessory elements) of a desired sequence to create the expression vector.

In some embodiments, host cells transfected with the above-described rAAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector.

In some embodiments, the nucleotide sequence encoding the CRISPR protein components of the rAAV is codon optimized. This type of optimization can entail a mutation of an encoding nucleotide sequence to mimic the codon preferences of the intended host organism or cell while encoding the same CasX protein or other protein component. Thus, the codons can be changed, but the encoded protein remains unchanged. For example, if the intended host cell was a human cell, a human codon-optimized CasX-encoding nucleotide sequence could be used. The gene design can be performed using algorithms that optimize codon usage and amino acid composition appropriate for the host cell utilized in the production of the rAAV vector. In one method of the disclosure, a library of polynucleotides encoding the components of the constructs is created and then assembled, as described above. The resulting genes are then assembled and the resulting genes used to transform a host cell and produce and recover the rAAV compositions for evaluation of its properties, as described herein. In some embodiments, as described more fully below, the nucleotide sequence encoding the components of the rAAV are engineered to remove CpG dinucleotides in order to reduce the immunogenicity of the components, while retaining their functional characteristics.

In some embodiments, a nucleotide sequence encoding a gRNA is operably linked to a regulatory element. In some embodiments, a nucleotide sequence encoding a CasX protein is operably linked to a regulatory element. In other cases, the nucleotide encoding the CasX and gRNA are linked and are operably linked to a single regulatory element. Exemplary accessory elements include a transcription promoter, a transcription enhancer element, a transcription termination signal, internal ribosome entry site (IRES) or P2A peptide to permit translation of multiple genes from a single transcript, polyadenylation sequences to promote downstream transcriptional termination, sequences for optimization of initiation of translation, and translation termination sequences. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional accessory element (e.g., the promoter) is functional in a targeted cell type or targeted cell population. For example, in some cases, the transcriptional accessory element can be functional in eukaryotic cells, e.g., packaging host cells for the production of the rAAV vector. In some cases, the accessory element is a transcription activator that works in concert with a promoter to initiate transcription. By transcriptional activation, it is intended that transcription will be increased above basal levels in the target cell by 10-fold, by 100-fold, more usually by 1000-fold.

Non-limiting examples of Pol II promoters suitable for use in the transgene of the rAAV of the disclosure include, but are not limited to polyubiquitin C (UBC), cytomegalovirus (CMV), simian virus 40 (SV40), chicken beta-Actin promoter and rabbit beta-Globin splice acceptor site fusion (CAG), chicken β-actin promoter with cytomegalovirus enhancer (CB7), PGK, Jens Tornoe (JeT), GUSB, CBA hybrid (CBh), elongation factor-1 alpha (EF-1alpha), beta-actin, Rous sarcoma virus (RSV), silencing-prone spleen focus forming virus (SFFV), CMVd1 promoter, truncated human CMV (tCMVd2), minimal CMV promoter, chicken β-actin promoter, chicken β-actin promoter with cytomegalovirus enhancer (CB7), HSV TK promoter, Mini-TK promoter, minimal IL-2 promoter, GRP94 promoter, Super Core Promoter 1, Super Core Promoter 2, MLC, MCK, GRK1 protein promoter, Rho promoter, CAR protein promoter, hSyn Promoter, U1A promoter, Ribsomal Rpl and Rps promoters (e.g., hRpl30 and hRps18), CMV53 promoter, minimal SV40 promoter, CMV53 promoter, SFCp promoter, pJB42CAT5 promoter, MLP promoter, rhodopsin promoter, EFS promoter, MeP426 promoter, MecP2 promoter, MHCK7 promoter, beta-glucuronidase (GUSB), CK7 promoter, and CK8e promoter. In some embodiments, an rAAV construct of the disclosure comprises a Pol II promoter comprising a sequence of SEQ ID NOS: 3532-3562, 3714-3739, 3773-3778, and 9344-9350, or a sequence having at least about 70%, at least about 80%, at least about 85%, at least about 90%, at least about 95%, at least about 95%, at least about 96%, at least about 97%, at least about 98%, or at least about 99% identity thereto. In one embodiment, the Pol II promoter is EF-1alpha, wherein the promoter enhances transfection efficiency, the transgene transcription or expression of the CRISPR nuclease, the proportion of expression-positive clones and the copy number of the episomal vector in long-term culture. In one embodiment, the Pol II promoter is JeT, wherein the promoter enhances transfection efficiency, the transgene transcription or expression of the CRISPR nuclease, the proportion of expression-positive clones and the copy number of the episomal vector in long-term culture. In one embodiment, the Pol II promoter is U1A, wherein the promoter enhances transfection efficiency, the transgene transcription or expression of the CRISPR nuclease, the proportion of expression-positive clones and the copy number of the episomal vector in long-term culture. In one embodiment, the Pol II promoter is UbC, wherein the promoter enhances transfection efficiency, the transgene transcription or expression of the CRISPR nuclease, the proportion of expression-positive clones and the copy number of the episomal vector in long-term culture. In some embodiments, the Pol II promoter is a truncated version of the foregoing promoters. In some embodiments the Pol II promoter in an rAAV construct has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides. In some embodiments, the Pol II promoter in an rAAV construct has between about 40 to about 585 nucleotides, between about 100 to about 400 nucleotides, or between about 150 to about 300 nucleotides. In some embodiments, the rAAV constructs comprise polynucleic acids comprising the Pol II promoters of any of the foregoing embodiments of the paragraph, as well as the promoters of Table 7, and can be, in some cases, configured in relation to the other components of the constructs as depicted in any one of FIGS. 1, FIG. 25, FIG. 28, FIGS. 38-40, FIG. 47, or FIG. 75.

In some embodiments, an rAAV construct of the disclosure comprises a Pol II promoter with a linked intron, wherein the intron enhances the ability of the promoter to increase transfection efficiency, the transgene transcription or expression of the CRISPR nuclease, the proportion of expression-positive clones and the copy number of the episomal vector in long-term culture. Exemplary embodiments of such promoter-intron combinations are described in the Examples.

Non-limiting examples of Pol III promoters suitable for use in the transgene of the rAAV of the disclosure include, but are notlimited to human U6, human U6 variant, human U6 isoform variant, mini U61, mini U62, mini U63, BiH1 (Bidrectional H1 promoter), BiU6 (Bidirectional U6 promoter), gorilla U6, rhesus U6, human 7sk, and human H1 promoters. In some embodiments, the Pol III promoter comprises a sequence selected from the group consisting of SEQ ID NOS: 3563, 3566-3582, 3599-3602, 3740-3746, 4025, 4029, 4032, and 4743, or a sequence having at least about 70%, at least about 80%, at least about 85%, at least about 90%, at least about 95%, at least about 95%, at least about 96%, at least about 97%, at least about 98%, or at least about 99% identity thereto. In the foregoing embodiments, the Pol III promoter enhances the transcription of the gRNA encoded by the rAAV. In some embodiments, an rAAV construct of the disclosure comprises a Pol III promoter comprising a sequence as set forth in Table 8, or a sequence having at least 85%, at least 90%, at least 95%, at least 95%, at least 96%, at least 97%, at least 98%, or at least 99% identity thereto. In some embodiments, the Pol III promoter is a truncated version of the foregoing promoters. In some embodiments the Pol III promoter in an rAAV construct of the disclosure has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides. In some embodiments the Pol III promoter in an rAAV construct of the disclosure has between about 70 to about 245 nucleotides, between about 100 to about 220 nucleotides, or between about 120 to about 160 nucleotides. In some embodiments, the rAAV constructs comprise polynucleic acids encoding the Pol III promoters of any of the foregoing embodiments of the paragraph, as well as the promoters of Table 8, and can be, in some cases, configured in relation to the other components of the constructs as depicted in any one of FIG. 1, FIG. 25, FIG. 28, FIGS. 38-40, FIG. 47, or FIG. 75.

Selection of the appropriate promoter is well within the level of ordinary skill in the art, as it relates to controlling expression, e.g., for modifying a gene or other target nucleic acid. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

In some embodiments, the disclosure provides rAAV transgenes comprising promoters and gRNA oriented in the forward direction (i.e., 5′ to 3′) relative to the orientation of the sequence encoding the Class 2, Type V CRISPR protein. In such a case, the gRNA would be 3′ of the promoter in the transgene. In some embodiments, the disclosure provides rAAV transgenes comprising promoters and gRNA oriented in the reverse direction (i.e., 3′ to 5′) relative to the orientation of the sequence encoding the Class 2, Type V CRISPR protein. In such a case, the gRNA would be 5′ of the promoter in the transgene. Exemplary promoters in the reverse orientation are described in the Examples and Table 50 and transgene constructs incorporating promoters in various locations and orientations are portrayed schematically in FIG. 1, FIG. 25, FIG. 28, FIGS. 38-40, FIG. 47, or FIG. 75.

In some embodiments, the present disclosure provides a polynucleotide sequence wherein one or more components of the transgene are operably linked to (under the control of) an inducible promoter operable in a eukaryotic cell. Examples of inducible promoters may include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, kanamycin-regulated promoter, steroid-regulated promoter, metal-regulated promoter, estrogen receptor-regulated promoter, etc. Inducible promoters can therefore, in some embodiments, be regulated by molecules including, but not limited to, doxycycline, estrogen and/or an estrogen analog, IPTG, etc. Additional examples of inducible promoters include, without limitation, chemically/biochemically-regulated and physically-regulated promoters such as alcohol-regulated promoters, kanamycin-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR, etc.), tetracycline regulated promoters, (e.g., promoter systems including Tet Activators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Recombinant expression vectors of the disclosure can also comprise elements that facilitate robust expression components of the disclosure (e.g., the CasX or the gRNA). For example, recombinant expression vectors utilized in the rAAV constructs of the disclosure can include one or more of a polyadenylation signal (poly(A) signal), an intronic sequence or a post-transcriptional accessory element (PTRE) such as a woodchuck hepatitis post-transcriptional accessory element (WPRE). Non-limiting examples of PTRE suitable for the rAAV constructs of the disclosure include the sequences of SEQ ID NOS: 3615-3617 of Table 16, or a sequence having at least 85%, at least 90%, at least 95%, at least 95%, at least 96%, at least 97%, at least 98%, or at least 99% identity thereto. Exemplary poly(A) signal sequences suitable for inclusion in the expression vectors of the disclosure include hGH poly(A) signal (short), HSV TK poly(A) signal, synthetic polyadenylation signals, SV40 poly(A) signal, SV40 Late PolyA signal, β-globin poly(A) signal, β-globin poly(A) short, and the like. Non-limiting examples of poly(A) signals suitable for the rAAV constructs of the disclosure include the sequences of SEQ ID NOS: 2401-3401 of Table 12, or a sequence having at least 85%, at least 90%, at least 95%, at least 95%, at least 96%, at least 97%, at least 98%, or at least 99% identity thereto. Non-limiting examples of introns suitable for the rAAV of the disclosure include the sequences of SEQ ID NOS: 3487-3531 of Table 22, or a sequence having at least 85%, at least 90%, at least 95%, at least 95%, at least 96%, at least 97%, at least 98%, or at least 99% identity thereto. A person of ordinary skill in the art will be able to select suitable elements to include in the recombinant expression vectors described herein.

The polynucleotides encoding the transgene components can be individually cloned into the rAAV expression vector. In some embodiments, the polynucleotide is a recombinant expression vector that comprises a nucleotide sequence encoding a CasX protein. In other embodiments, the disclosure provides a recombinant expression vector comprising a polynucleotide sequence encoding a CasX protein and a nucleotide sequence encoding a first gRNA with a linked targeting sequence complementary to a target nucleic acid of a cell, and, optionally, a second gRNA with a linked targeting sequence complementary to different or overlapping regions of a target nucleic acid of a cell. In some cases, the nucleotide sequence encoding the CasX protein variant and/or the nucleotide sequence encoding the gRNA are each operably linked to a promoter that is operable in a cell type of choice. In other embodiments, the nucleotide sequence encoding the CasX protein variant and the nucleotide sequence encoding the gRNA are provided in separate vectors.

The nucleic acid sequences encoding the transgene components are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available.

The recombinant expression vectors can be delivered to the target host cells by a variety of methods, as described more fully, below, and in the Examples. Such methods include, e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, cell squeezing, calcium phosphate precipitation, direct microinjection, nanoparticle-mediated nucleic acid delivery, and the like. A number of transfection techniques are generally known in the art; see, e.g., Sambrook et al. (1989) Molecular Cloning, a laboratory manual, Cold Spring Harbor Laboratories, New York. Packaging cells are typically used to form virus particles; such cells include BHK cells, HEK293 cells, HEK293T cells, NSO cells, SP2/0 cells, YO myeloma cells, P3X63 mouse myeloma cells, PER cells, PER.C6 cells, hybridoma cells, NIH3T3 cells, COS cells, HeLa cells, and CHO cells (and other cells known in the art), which package adenovirus, which are then recovered by conventional methods known in the art.

In some embodiments, host cells transfected with the above-described rAAV expression vectors are rendered capable of providing rAAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. In some embodiments, packaging cells are transfected with plasmids comprising AAV helper functions to complement necessary AAV functions that are missing from the rAAV expression vectors. Thus, AAV helper function plasmids include one, or both of the major AAV ORFs (open reading frames), encoding the rep and cap coding regions, the aap (assembly) gene, or functional homologues thereof, and the adenoviral helper genes comprising E2A, E4, and VA genes, operably linked to a promoter. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation accessory elements, including constitutive and inducible promoters, transcription enhancer elements, transcription terminators, etc., may be used in the expression vector.

### VII. Therapeutic Methods

The present disclosure provides methods of treating a disease in a subject in need thereof. In some embodiments of the method, the subject has one or more mutations in a gene, wherein administration of the rAAV is administered to modify the gene, either to knock down or knock out expression of the gene product. In some embodiments of the method, the rAAV is administered to correct a mutation in a gene of the subject. In some embodiments, the methods of the disclosure can prevent, treat and/or ameliorate a disease of a subject by the administering to the subject of an rAAV composition of the disclosure. In some embodiments, the composition administered to the subject further comprises pharmaceutically acceptable carrier, diluent or excipient.

In some embodiments, the disclosure provides methods of treating a disease in a subject in need thereof comprising modifying a target nucleic acid in a cell of the subject, the modifying comprising administering to the subject a therapeutically effective dose of an rAAV of any of the embodiments described herein wherein the targeting sequence of the encoded gRNA has a sequence that hybridizes with the target nucleic acid, resulting in the modification of the target nucleic acid by the CasX protein.

In other embodiments, the methods of treating a disease in a subject in need thereof comprise administering to the subject a therapeutically effective dose of an rAAV of any of the embodiments described herein wherein the targeting sequence of the encoded gRNA has a sequence that hybridizes with the target nucleic acid and wherein the rAAV further comprises a donor template comprises one or more mutations or a heterologous sequence that is inserted into or replaces the target nucleic acid sequence to knock-down or knock-out the gene comprising the target nucleic acid. In the foregoing, the insertion of the donor template serves to disrupt expression of the gene and the resulting gene product. In some embodiments of the foregoing methods, the donor DNA template ranges in size from 10-5,000 nucleotides. In other embodiments of the foregoing methods, the donor template ranges in size from 100-1,000 nucleotides. In some cases, the donor template is a single-stranded RNA or DNA template.

The modified cell of the treated subject can be a eukaryotic cell selected from the group consisting of a rodent cell, a mouse cell, a rat cell, a primate cell, a non-human primate cell, and a human cell. In some embodiments, the eukaryotic cell of the treated subject is a human cell.

In some embodiments, the method comprises administering to the subject the rAAV of the embodiments described herein via an administration route selected from the group consisting of subcutaneous, intradermal, intraneural, intranodal, intramedullary, intramuscular, intralumbar, intrathecal, subarachnoid, intraventricular, intracapsular, intravenous, intralymphatical, intraocular or intraperitoneal routes, wherein the administering method is injection, transfusion, or implantation. In some embodiments of the methods of treating a disease in a subject, the subject is selected from the group consisting of mouse, rat, pig, non-human primate, and human. In a particular embodiment, the subject is a human.

In some embodiments of the method of treating a disease in a subject in need thereof, the rAAV is administered at a dose of at least about 1×10⁵ vector genomes/kg (vg), at least about 1×10⁶ vector genomes/kilogram (vg/kg), at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, at least about 1×101⁶ vg/kg. In other embodiments of the method of treatment, the rAAV is administered to a subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

In organ systems like the eye, the rAAV is administered at a dose of at least about 1×10⁵ vector genomes (vg), at least about 1×10⁶ vg, at least about 1×10⁷ vg, at least about 1×10⁸ vg, at least about 1×10⁹ vg, at least about 1×10¹⁰ vg, at least about 1×10¹¹ vg, at least about 1×10¹² vg, at least about 1×10¹³ vg, at least about 1×10¹⁴ vg, at least about 1×10¹⁵ vg, at least about 1×10¹⁶ vg.

A number of therapeutic strategies have been used to design the compositions for use in the methods of treatment of a subject with a disease. In some embodiments, the invention provides a method of treatment of a subject having a disease, the method comprising administering to the subject an rAAV of any of the embodiments disclosed herein according to a treatment regimen comprising one or more consecutive doses using a therapeutically effective dose. In some embodiments of the treatment regimen, the therapeutically effective dose of the rAAV is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of subcutaneous, intradermal, intraneural, intranodal, intramedullary, intramuscular, intralumbar, intrathecal, subarachnoid, intraventricular, intracapsular, intravenous, intralymphatical, intraocular, subretinal, intravitreal, or intraperitoneal routes, wherein the administering method is injection, transfusion, or implantation.

In some embodiments, the administering of the therapeutically effective amount of an rAAV to knock down or knock out expression of a gene having one or more mutations leads to the prevention or amelioration of the underlying disease such that an improvement is observed in the subject, notwithstanding that the subject may still be afflicted with the underlying disease. In some embodiments, the administration of the therapeutically effective amount of the rAAV leads to an improvement in at least one clinically-relevant parameter for the disease. In some embodiments of the method of treatment, the subject is selected from mouse, rat, pig, dog, non-human primate, and human.

In some embodiments, the disclosure provides compositions of any of the rAAV embodiments described herein for the manufacture of a medicament for the treatment of a human in need thereof. In some embodiments, the medicament is administered to the subject according to a treatment regimen comprising one or more consecutive doses using a therapeutically effective dose.

VIII. rAAV Engineered to Reduce Immunogenicity Retain Editing Properties

rAAV-associated pathogen associated molecular patterns (PAMPs) that contribute to immune responses in mammalians hosts include: i) ligands present on rAAV viral capsids that bind toll-like receptor 2 (TLR2), a cell-surface PRR on non-parenchymal cells in the liver; and ii) unmethylated CpG dinucleotides in viral DNA that bind TLR9, an endosomal PRR in plasmacytoid dendritic cells (pDCs) and B cells (Faust, S M, et al. CpG-depleted adeno-associated virus vectors evade immune detection. J. Clinical Invest. 123:2294 (2013)). In particular, CpG dinucleotide motifs (CpG PAMPs) in AAV vectors are immunostimulatory because of their high degree of hypomethylation, relative to mammalian CpG motifs, which have a high degree of methylation. Accordingly, reducing the frequency of unmethylated CpGs in rAAV genomes to a level below the threshold that activates human TLR9 is expected to reduce the immune response to exogenously administered rAAV-based biologics. Similarly, methylation of CpG PAMPs in rAAV constructs is similarly expected to reduce the immune response to rAAV-based biologics.

In some embodiments, the present disclosure provides rAAV wherein one or more components of the transgene are optimized for depletion of CpG dinucleotides by the substitution of homologous nucleotide sequences from mammalian species, wherein the one or more components substantially retain their functional properties upon expression in a transduced cell; e.g., ability to drive expression of the CRISPR nuclease, ability to drive expression of the gRNA, enhance the expression of the CRISPR nuclease and/or the gRNA, and enhanced ability to edit a target nucleic acid sequence. In some embodiments, the present disclosure provides rAAV wherein one or more rAAV transgene component sequences selected from the group consisting of 5′ ITR, 3′ ITR, Pol III promoter, Pol II promoter, encoding sequence for CRISPR nuclease, encoding sequence for gRNA, 3′ UTR, poly(A) signal sequence, and accessory element are optimized for depletion of all or a portion of the CpG dinucleotides, wherein the resulting rAAV transgene is substantially devoid of CpG dinucleotides. In some embodiments, the present disclosure provides rAAV wherein one or more rAAV transgene component sequences selected from the group consisting of 5′ ITR, 3′ ITR, Pol III promoter, Pol II promoter, encoding sequence for a CRISPR nuclease, encoding sequence for gRNA, poly(A) signal, and accessory element comprise less than about 10%, less than about 5%, or less than about 1% CpG dinucleotides. In some embodiments, the present disclosure provides rAAV wherein one or more rAAV transgene component sequences selected from the group consisting of 5′ ITR, 3 ITR, Pol III promoter, Pol II promoter, encoding sequence for the CRISPR nuclease, encoding sequence for the gRNA, and poly(A) signal are devoid of CpG dinucleotides. In some embodiments, the present disclosure provides rAAV wherein the transgene comprises less than about 10%, less than about 5%, or less than about 1% CpG dinucleotides. In some embodiments, the present disclosure provides rAAV wherein the one or more rAAV component sequences optimized for depletion of CpG dinucleotides are selected from the group of sequences consisting of SEQ ID NOS: 9327-9333, 9369-9380, and 3735-3772 or a sequence having at least about 70%, at least about 80%, at least about 90%, at least about 95%, at least about 96%, at least about 97%, at least about 98%, or at least about 99% sequence identity thereto. In some embodiments, the present disclosure provides rAAV wherein the sequence encoding the CasX nuclease protein component sequences are optimized for depletion of CpG dinucleotides, selected from the group consisting of SEQ ID NOS: 9327-9333 and 9369-9380, or a sequence having at least about 70%, at least about 80%, at least about 90%, at least about 95%, at least about 96%, at least about 97%, at least about 98%, or at least about 99% sequence identity thereto. In some embodiments, the disclosure provides a CpG-depleted polynucleotide sequence encoding a gRNA scaffold, wherein the sequence is selected from the group consisting of SEQ ID NOS: 3751-3772, or a sequence having at least about 70%, at least about 80%, at least about 90%, at least about 95%, at least about 97%, at least about 98%, or at least about 99% sequence identity thereto. In some embodiments, the disclosure provides a CpG-depleted polynucleotide sequence encoding an ITR, wherein the sequence is selected from the group consisting of SEQ ID NOS: 3749 and 3750. In some embodiments, the disclosure provides a CpG-depleted polynucleotide sequence encoding a promoter, wherein the sequence is selected from the group consisting of SEQ ID NOS: 3735-3746. In some embodiments, the disclosure provides a CpG-depleted polynucleotide sequence encoding a poly(A) signal sequence, wherein the sequence is SEQ ID NO: 3748. In some embodiments, the disclosure provides rAAV having one or more components of the transgene optimized for depletion of CpG dinucleotides, wherein the expressed CRISPR nuclease and gRNA retain at least about 60%, at least about 70%, at least about 80%, or at least about 90% of the editing potential for a target nucleic acid compared to an rAAV wherein the transgene has not been optimized for depletion of CpG dinucleotides, when assayed in an in vitro assay under comparable conditions. In a particular embodiment, the present disclosure provides rAAV wherein the one or more rAAV component sequences optimized for depletion of CpG dinucleotides that retain editing potential are selected from the group of sequences consisting of SEQ ID NOS: 9327-9333, 9369-9380, and 3735-3772, or a sequence having at least about 80%, at least about 80%, at least about 90%, at least about 95%, at least about 96%, at least about 97%, at least about 98%, or at least about 99% sequence identity thereto.

The embodiments of the rAAV comprising the one or more components of the transgene optimized for depletion of CpG dinucleotides have, as an improved characteristic, a lower potential for inducing an immune response, either in vivo (when administered to a subject) or in in vitro mammalian cell assays designed to detect markers of an inflammatory response. In some embodiments, the administration of a therapeutically effective dose of the rAAV comprising the one or more components of the transgene optimized for depletion of CpG dinucleotides to a subject results in a reduced immune response compared to the immune response of a comparable rAAV wherein the transgene has not been optimized for depletion of CpG dinucleotides, wherein the reduced response is determined by the measurement of one or more parameters such as production of antibodies or a delayed-type hypersensitivity to an rAAV component, or the production of inflammatory cytokines and markers, such as, but not limited to TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF). In some embodiments, the rAAV comprising the one or more components of the transgene that are substantially devoid of CpG dinucleotides elicits reduced production of one or more inflammatory markers selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF) of at least about 10%, at least about 20%, at least about 30%, at least about 40%, at least about 50%, at least about 60%, at least about 80%, or at least about 90% compared to the comparable rAAV that is not CpG depleted, when assayed in a cell-based vitro assay using cells known in the art appropriate for such assays; e.g., monocytes, macrophages, T-cells, B-cells, etc. In a particular embodiment, the rAAV comprising the one or more components of the transgene optimized for depletion of CpG dinucleotides exhibits a reduced activation of TLR9 in hNPCs in an in vitro assay of at least about 10%, at least about 20%, at least about 30%, at least about 40%, at least about 50%, at least about 60%, at least about 80%, or at least about 90% compared to the comparable rAAV that is not CpG depleted.

### IX. Kits and Articles of Manufacture

In other embodiments, provided herein are kits comprising an rAAV of any of the embodiments of the disclosure, and a suitable container (for example a tube, vial or plate).

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

### ENUMERATED EMBODIMENTS

The following sets of enumerated embodiments are included for illustrative purposes and are not intend to limit the scope of the invention.

### Set I

Embodiment I-1. A polynucleotide comprising the following component sequences:
a. a first AAV inverted terminal repeat (ITR) sequence as disclosed in the present disclosure;
b. a second AAV ITR sequence as disclosed in the present disclosure;
c. a first promoter sequence as disclosed in the present disclosure;
d. a sequence encoding a CRISPR protein as disclosed in the present disclosure;
e. a sequence encoding a first guide RNA (gRNA) as disclosed in the present disclosure; and,
f. optionally, at least one accessory element sequence as disclosed in the present disclosure, wherein the polynucleotide is configured for incorporation into a recombinant adeno-associated virus (AAV).

Embodiment I-2. The polynucleotide of embodiment I-1, wherein the first AAV ITR, the second AAV ITR, the first promoter sequence, the sequence encoding the CRISPR protein, the sequence encoding the first gRNA, the at least one accessory element sequence, or a combination thereof, is modified to reduce or deplete at least one CpG dinucleotide.

Embodiment I-3. The polynucleotide of embodiment I-1 or embodiment I-2, wherein the first promoter sequence is a muscle-specific promoter.

Embodiment I-4. The polynucleotide of any one of embodiments 1-3, wherein the accessory element sequence encodes a muscle-specific accessory element.

Embodiment I-5. The polynucleotide of any one of embodiments 1-4, wherein the gRNA is modified to exhibit improved activity for double strand DNA cleavage.

Embodiment I-6. The polynucleotide of any one of embodiments 1-5 wherein the CRISPR protein is modified to exhibit improved activity for double strand DNA cleavage or spacer specificity at TTC, ATC, or CTC PAM sequences.

### Set II

Embodiment II-1. A recombinant adeno-associated virus (rAAV) transgene wherein
a. the transgene comprises:
   i) a polynucleotide sequence encoding a CasX protein comprising a sequence selected from the group consisting of SEQ ID NOS: 137-512, 9382-9542, and 9607-9609, or a sequence having at least about 70% sequence identity thereto; and
   ii) a polynucleotide sequence encoding a first guide RNA (gRNA) comprising a targeting sequence of 15 to 20 nucleotides complementary to a target nucleic acid of a cell;
b. the transgene has less than about 4700 nucleotides; and
c. the rAAV transgene is configured for incorporation into a rAAV capsid.

Embodiment II-2. The rAAV transgene of embodiment II-1, wherein the CasX protein comprises a sequence selected from the group consisting of SEQ ID NOS: 137-512, 9382-9542 and 9607-9609.

Embodiment II-3. The rAAV of embodiment II-1 or II-2, wherein the wherein the CasX protein is a CasX variant selected from the group consisting of SEQ ID NOS: 9385, 9391, 9393, 9401, 9409, 9417, 9419, 9423, 9429, 9443, 9444, 9447, 9449, 9450, 9452, 9453, 9455, 9456, 9458, 9462, 9466, 9469, 9470, 9472, 9478, 9483, 9485, 9491, 9495, 9499, 9501, 9512, 9513, 9517, 9519, 9521, 9536, 9542, 9607, and 9609, wherein the encoded CasX variant exhibits improved editing of a target nucleic acid in an in vitro assay, compared to a CasX variant of SEQ ID NO: 197 and assayed under comparable conditions.

Embodiment II-4. The rAAV of embodiment II-1 or II-2, wherein the wherein the CasX protein is a CasX variant selected from the group consisting of SEQ ID NOS: 9385, 9386, 9388, 9390, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9486, 9487, 9488, 9492, 9493, 9496, 9509, 9512, 9516, 9517, 9519, 9521, 9522, 9529, 9536, 9542, 9608, and 9609, wherein the encoded CasX variant exhibits improved editing specificity of a target nucleic acid in an in vitro assay, compared to a CasX variant of SEQ ID NO: 197 and assayed under comparable conditions.

Embodiment II-5. The rAAV of embodiment II-1 or II-2, wherein the wherein the CasX protein is a CasX variant selected from the group consisting of SEQ ID NOS: 9385, 9386, 9388, 9390, 9393, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9483, 9486, 9488, 9491, 9492, 9493, 9495, 9496, 9509, 9512, 9513, 9516, 9517, 9519, 9521, 9522, 9529, 9536, 9542, 9608, and 9609, wherein the encoded CasX variant exhibits improved editing specificity ratio of a target nucleic acid in an in vitro assay, compared to a CasX variant of SEQ ID NO: 197 and assayed under comparable conditions.

Embodiment II-6. The rAAV of embodiment II-1 or II-2, wherein the wherein the CasX protein is a CasX variant selected from the group consisting of SEQ ID NOS: 9385, 9409, 9417, 9443, 9444, 9447, 9450, 9452, 9455, 9466, 9469, 9470, 9472, 9478, 9512, 9513, 9517, 9519, 9521, 9536, 9542, and 9609, wherein the encoded CasX variant exhibits improved editing and improved specificity of a target nucleic acid in an in vitro assay, compared to a CasX variant of SEQ ID NO: 197 and assayed under comparable conditions.

Embodiment II-7. The rAAV of embodiment II-1 or II-2, wherein the wherein the CasX protein is a CasX variant selected from the group consisting of SEQ ID NOS: 9385, 9393, 9409, 9417, 9443, 9444, 9447, 9450, 9452, 9455, 9466, 9469, 9470, 9472, 9478, 9483, 9491, 9495, 9512, 9513, 9517, 9519, 9521, 9536, 9542, and 9609, wherein the encoded CasX variant exhibits improved editing and improved specificity ratio of a target nucleic acid in an in vitro assay, compared to a CasX variant of SEQ ID NO: 197 and assayed under comparable conditions.

Embodiment II-8. The rAAV transgene of embodiment II-2, wherein the CasX protein comprises a sequence selected from the group consisting of SEQ ID NOS: 190 and 197.

Embodiment II-9. The rAAV transgene of any one of embodiments II-1 to II-7, wherein the transgene further comprises one or more components selected from the group consisting of:
a. a first and a second rAAV inverted terminal repeat (ITR) sequence;
b. a first promoter sequence operably linked to the Type V CRISPR protein;
c. a sequence encoding a nuclear localization signal (NLS);
d. a 3′ UTR;
e. a poly(A) signal sequence;
f. a second promoter operably linked to the first gRNA; and
g. an accessory element.

Embodiment II-10. The rAAV transgene of embodiment II-9, wherein the first promoter is a pol II promoter selected from the group consisting of polyubiquitin C (UBC) promoter, cytomegalovirus (CMV) promoter, simian virus 40 (SV40) promoter, chicken beta-Actin promoter and rabbit beta-Globin splice acceptor site fusion (CAG), chicken β-actin promoter with cytomegalovirus enhancer (CB7), PGK promoter, Jens Tornoe (JeT) promoter, GUSB promoter, CBA hybrid (CBh) promoter, elongation factor-1 alpha (EF-1alpha) promoter, beta-actin promoter, Rous sarcoma virus (RSV) promoter, silencing-prone spleen focus forming virus (SFFV) promoter, CMVd1 promoter, truncated human CMV (tCMVd2) promoter, minimal CMV promoter, hepB promoter, chicken β-actin promoter, HSV TK promoter, Mini-TK promoter, minimal IL-2 promoter, GRP94 promoter, Super Core Promoter 1, Super Core Promoter 2, Super Core Promoter 3, adenovirus major late (AdML) promoter, MLC promoter, MCK promoter, GRK1 protein promoter, Rho promoter, CAR protein promoter, hSyn Promoter, U1a promoter, Ribosomal Protein Large subunit 30 (Rp130) promoter, Ribosomal Protein Small subunit 18 (Rps18) promoter, CMV53 promoter, minimal SV40 promoter, CMV53 promoter, SFCp promoter, Mecp2 promoter, pJB42CAT5 promoter, MLP promoter, EFS promoter, rhodopsin promoter, MeP426 promoter, MecP2 promoter, Desmin promoter, MHCK promoter, MHCK7 promoter, beta-glucuronidase (GUSB) promoter, CK7 promoter, and CK8e promoter.

Embodiment II-11. The rAAV transgene of embodiment II-9 or II-10, wherein the first promoter is a pol II promoter selected from the group consisting of U1A, UbC, and JeT.

Embodiment II-12. The rAAV transgene of any one of embodiments II-9 to II-13, wherein the first promoter comprises a sequence selected from the group consisting of SEQ ID NOS: 3532-3562, 3714-3739, 3773-3778, and 9344-9350, or a sequence having at least about 70%, at least about 80%, at least about 85%, at least about 90%, at least about 95%, at least about 95%, at least about 96%, at least about 97%, at least about 98%, or at least about 99% identity thereto.

Embodiment II-13. The polynucleotide of any one of embodiments II-9 to II-12, wherein the first promoter sequence has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides.

Embodiment II-14. The rAAV transgene of any one of embodiments II-9, wherein the second promoter is a pol III promoter selected from the group consisting of human U6 promoter, human U6 variant promoter, human U6 isoform variant promoter, mini U61 promoter, mini U62 promoter, mini U63 promoter, BiH1 (Bidrectional H1 promoter), BiU6 (Bidirectional U6 promoter), gorilla U6 promoter, rhesus U6 promoter, human 7sk promoter, and human H1 promoter.

Embodiment II-15. The rAAV transgene of embodiment II-14, wherein the second promoter is a pol III promoter selected from the group consisting of human U6, human U6 variant, or human U6 isoform variant.

Embodiment II-16. The rAAV transgene of embodiment II-15, wherein the second promoter comprises a sequence selected from the group consisting of SEQ ID NOS: 3563, 3566-3582, 3599-3602, 3740-3746, 4025, 4029, 4032, and 4743 or a sequence having at least about 70%, at least about 80%, at least about 85%, at least about 90%, at least about 95%, at least about 95%, at least about 96%, at least about 97%, at least about 98%, or at least about 99% identity thereto.

Embodiment II-17. The rAAV transgene of any one of embodiments II-14 to II-16, wherein the second promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

Embodiment II-18. The rAAV transgene of any one of embodiments II-9, wherein the poly(A) signal sequence is selected from the group consisting of SEQ ID NOS: 2401-3401, or a sequence having at least about 70%, at least about 80%, at least about 85%, at least about 90%, at least about 95%, at least about 95%, at least about 96%, at least about 97%, at least about 98%, or at least about 99% identity thereto.

Embodiment II-19. The rAAV transgene of any one of embodiments II-9, wherein the encoded NLS comprises a sequence selected from the group consisting of SEQ ID NOS: 3411-3486, 3939-3971, and 4065-4111.

Embodiment II-20. The rAAV transgene of any one of embodiments II-1 to II-19, wherein the transgene comprises a polynucleotide sequence encoding a second gRNA with a linked targeting sequence of 15 to 20 nucleotides complementary to a different or overlapping region of a target nucleic acid of a cell, as compared to the targeting sequence of the first gRNA.

Embodiment II-21. The rAAV transgene of any one of embodiments II-1 to II-20, wherein the first and/or the second gRNA each comprise:
a. a scaffold sequence selected from the group consisting of SEQ ID NOS: 2238-2400, 9257-9289 and 9588, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto; or
b. a scaffold sequence selected from the group consisting of SEQ ID NOS: 2238-2400, 9257-9289 and 9588, further comprising at least 1, 2, 3, 4, or 5 mismatches thereto.

Embodiment II-22. The rAAV transgene of embodiment II-20 or II-21, wherein the first and the second gRNA each comprise a scaffold sequence of SEQ ID NO: 2293 or SEQ ID NO: 9588.

Embodiment II-23. The rAAV transgene of any one of embodiments II-20 to II-22, comprising a third promoter operably linked to the second gRNA.

Embodiment II-24. The rAAV transgene of embodiment II-23, wherein the third promoter is a pol III promoter selected from the group consisting of human U6, human U6 variant, human U6 isoform variant, mini U61, mini U62, mini U63, BiH1 (Bidirectional H1 promoter), BiU6 (Bidirectional U6 promoter), gorilla U6, rhesus U6, human 7sk, and human H1 promoters.

Embodiment II-25. The rAAV transgene of embodiment II-23, wherein the third promoter is a pol III promoter selected from the group consisting of human U6, human U6 variant, and human U6 isoform variant.

Embodiment II-26. The rAAV transgene of any one of embodiments II-23 to II-25, wherein the third promoter comprises a sequence selected from the group consisting of SEQ ID NOS: 3563, 3566-3582, 3599-3602, 3740-3746, 4025, 4029, 4032, and 4743, or a sequence having at least about 70%, at least about 80%, at least about 85%, at least about 90%, at least about 95%, at least about 95%, at least about 96%, at least about 97%, at least about 98%, or at least about 99% identity thereto.

Embodiment II-27. The rAAV transgene of any one of embodiments II-23 to II-26, wherein the third promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

Embodiment II-28. The rAAV transgene of any one of embodiments II-20 to II-27, wherein:
a. the polynucleotide sequence encoding the first gRNA and the polynucleotide sequence encoding the second gRNA are 5′ of the polynucleotide sequence encoding the CasX protein;
b. the polynucleotide sequence encoding the first gRNA is 5′ of the polynucleotide sequence encoding the CasX protein and the polynucleotide sequence encoding the second gRNA is 3′ of the polynucleotide sequence encoding the CasX protein;
c. the polynucleotide sequence encoding the first gRNA is 3′ of the polynucleotide sequence encoding the CasX protein and the polynucleotide sequence encoding the second gRNA is 5′ of the polynucleotide sequence encoding the CasX protein; or
d. the polynucleotide sequence encoding the first gRNA and the polynucleotide sequence encoding the second gRNA are 3′ of the polynucleotide sequence encoding the CasX protein.

Embodiment II-29. The rAAV transgene of any one of embodiments II-20 to II-28, wherein:
a. the polynucleotide sequence encoding the first gRNA and the polynucleotide sequence encoding the second gRNA are encoded in a forward orientation relative to the polynucleotide sequence encoding the CasX protein;
b. the polynucleotide sequence encoding the first gRNA is encoded in a forward orientation relative to the polynucleotide sequence encoding the CasX protein and the polynucleotide sequence encoding the second gRNA is encoded in a reverse orientation relative to the polynucleotide sequence encoding the CasX protein;
c. the polynucleotide sequence encoding the first gRNA is encoded in a reverse orientation relative to the polynucleotide sequence encoding the CasX protein and the polynucleotide sequence encoding the second gRNA is encoded in a forward orientation relative to the polynucleotide sequence encoding the CasX protein; or
d. the polynucleotide sequence encoding the first gRNA and the polynucleotide sequence encoding the second gRNA are encoded in a reverse orientation relative to the polynucleotide sequence encoding the CasX protein.

Embodiment II-30. The rAAV transgene of any one of embodiments II-20 to II-29, wherein the transgene has less than about 4800, less than about 4750, less than about 4700, less than about 4650 nucleotides, or less than about 4600 nucleotides.

Embodiment II-31. The rAAV transgene of any one of embodiments II-20 to II-30, wherein the rAAV transgene is configured for incorporation into an rAAV capsid.

Embodiment II-32. The rAAV transgene of any one of embodiments II-1 to II-31, wherein one or more components of the transgene are optimized to reduce or deplete CpG motifs.

Embodiment II-33. The rAAV transgene of embodiment II-32, wherein the one or more components comprise less than about 10%, less than about 5%, or less than about 1% CpG dinucleotides.

Embodiment II-34. The rAAV transgene of embodiment II-32 or II-33, wherein the CpG-depleted polynucleotide sequence encoding the CasX protein is selected from the group consisting of SEQ ID NOS: 9327-9333 and 9369-9380.

Embodiment II-35. The rAAV transgene of embodiment II-32 or II-33, wherein the CpG-depleted polynucleotide sequence encodes a gRNA scaffold, and is selected from the group consisting of SEQ ID NOS: 3751-3772.

Embodiment II-36. The rAAV transgene of embodiment II-32 or II-33, wherein the CpG-depleted polynucleotide sequence of the ITR is selected from the group consisting of SEQ ID NOS: 3749 and 3750.

Embodiment II-37. The rAAV transgene of embodiment II-32 or II-33, wherein the CpG-depleted polynucleotide sequence of the promoter is selected from the group consisting of SEQ ID NOS: 3735-3746.

Embodiment II-38. The rAAV transgene of embodiment II-32 or II-33, wherein the CpG-depleted polynucleotide sequence of the poly(A) signal is SEQ ID NO: 3748.

Embodiment II-39. The rAAV transgene of any one of embodiments II-1 to II-38, wherein the transgene has the configuration of a construct depicted in any one of FIGS. 1, 25, 28, 38-40, 47 and 75.

Embodiment II-40. A recombinant adeno-associated virus (rAAV) comprising:
a. an AAV capsid protein, and
b. the transgene of any one of embodiments II-1 to II-39.

Embodiment II-41. The rAAV of embodiment II-40, wherein the AAV capsid protein is derived from serotype AAV1, AAV2, AAV3, AAV4, AAV5, AAV6, AAV7, AAV8, AAV9, AAV10, AAV 11, AAV12, AAV 9.45, AAV 9.61, AAV 44.9, AAV-Rh74, AAVRh10, MyoAAV 1A1, MyoAAV 1A2, or MyoAAV 2A.

Embodiment II-42. The rAAV of embodiment II-41, wherein the AAV capsid protein and the 5′ and 3′ ITR are derived from the same serotype of AAV.

Embodiment II-43. The rAAV of embodiment II-41, wherein the AAV capsid protein and the 5′ and 3′ ITR are derived from different serotypes of AAV.

Embodiment II-44. The rAAV of embodiment II-43, wherein the 5′ and 3′ ITR are derived from AAV serotype 2.

Embodiment II-45. The rAAV of any one of embodiments II-40 to II-44, wherein upon transduction of a cell with the rAAV, the CasX protein and the first and/or the second gRNA encoded in the rAAV transgene are expressed.

Embodiment II-46. The rAAV of embodiment II-45, wherein upon expression, the first and/or the second gRNA is capable of forming a ribonucleoprotein (RNP) complex with the CasX protein.

Embodiment II-47. The rAAV of embodiment II-46, wherein the RNP is capable of binding and modifying a target nucleic acid of the cell.

Embodiment II-48. The rAAV of any one of embodiments II-40 to II-47, wherein inclusion of a poly(A) signal in the transgene enhances expression of the CasX protein and editing efficiency of a target nucleic acid in a cell transduced by the rAAV.

Embodiment II-49. The rAAV of any one of embodiments II-40 to II-47, wherein inclusion of a posttranscriptional regulatory element (PTRE) accessory element in the transgene enhances editing efficiency of a target nucleic acid in a cell transduced by the rAAV.

Embodiment II-50. The rAAV of embodiment II-49, wherein the PTRE comprises a sequence selected from the group consisting of SEQ ID NOS: 3615-3617, or a sequence having at least about 70%, at least about 80%, at least about 85%, at least about 90%, at least about 95%, at least about 95%, at least about 96%, at least about 97%, at least about 98%, or at least about 99% identity thereto.

Embodiment II-51. The rAAV of any one of embodiments II-40 to II-50, wherein components of the transgene modified for depletion of all or a portion of the CpG dinucleotides exhibit a lower potential for inducing an immune response in a cell transduced with the rAAV, compared to a rAAV wherein the components are not modified for depletion of the CpG dinucleotides.

Embodiment II-52. The rAAV of embodiment II-51, wherein the lower potential for inducing an immune response is exhibited in an in vitro mammalian cell assay designed to detect production of one or more markers of an inflammatory response selected from the group consisting of TLR9, interleukin-1 (IL-1), IL-6, IL-12, IL-18, tumor necrosis factor alpha (TNF-a), interferon gamma (IFNγ), and granulocyte-macrophage colony stimulating factor (GM-CSF).

Embodiment II-53. The rAAV of embodiment II-51 or II-52, wherein the rAAV comprising the component sequences modified for depletion of all or a portion of the CpG dinucleotides elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

Embodiment II-54. The rAAV of any one of embodiments II-51 to II-53, wherein the expressed CasX and the first and/or the second gRNA retain at least about 60%, at least about 70%, at least about 80%, or at least about 90% of the editing potential for a target nucleic acid compared to an rAAV wherein the transgene has not been optimized for depletion of CpG dinucleotides, when assayed in an in vitro assay under comparable conditions.

Embodiment II-55. The rAAV of embodiment II-40, wherein incorporation of a Pol II promoter selected from the group consisting of CK8e, MHCK7, and MHCK in the transgene of a rAAV used to transduce a muscle cell results in higher expression of the CasX protein in the muscle cell compared to incorporation of a UbC promoter.

Embodiment II-56. The rAAV of embodiment II-40, wherein incorporation of a muscle enhancer sequence selected from the group consisting of SEQ ID NOS: 3779-3809 in the transgene of a rAAV used to transduce a muscle cell results in higher expression of the CasX protein in the muscle cell compared to a rAAV not incorporating the muscle enhancer.

Embodiment II-57. A method for modifying a target nucleic acid of a gene in a population of mammalian cells, comprising contacting a plurality of the cells with an effective amount of the rAAV of any one of embodiments II-40 to II-56, wherein the target nucleic acid of the gene targeted by the first and/or the second gRNA is modified by the expressed CasX protein.

Embodiment II-58. The method of embodiment II-57, wherein the gene comprises one or more mutations.

Embodiment II-59. The method of embodiment II-57 or II-58, wherein the modifying comprises introducing an insertion, deletion, substitution, duplication, or inversion of one or more nucleotides in the target nucleic acid of the cells of the population.

Embodiment II-60. The method of any one of embodiments II-57 to II-59, wherein the gene is knocked down or knocked out.

Embodiment II-61. The method of any one of embodiments II-57 to II-59, wherein the gene is modified such that a functional gene product can be expressed.

Embodiment II-62. The method of any one of embodiments II-57 to II-61, wherein the rAAV comprises the first and the second gRNA, wherein the second gRNA comprises a targeting sequence complementary to a different target site in a gene targeted by the targeting sequence of the first gRNA, wherein the nucleotides between the target sites are excised by cleavage of the target sites by the CasX protein.

Embodiment II-63. The method of any one of embodiments II-57 to II-61, wherein the rAAV comprises the first and the second gRNA, wherein the second gRNA comprises a targeting sequence complementary to a target site in a different gene targeted by the targeting sequence of the first gRNA, wherein the target nucleic acid at each target site is modified by the CasX protein.

Embodiment II-64. A method of treating a disease in a subject caused by one or more mutations in a gene of the subject, comprising administering a therapeutically effective dose of the rAAV of any one of embodiments II-40 to II-56 to the subject.

Embodiment II-65. The method of embodiment II-62, wherein the rAAV is administered to the subject by a route of administration selected from subcutaneous, intradermal, intraneural, intranodal, intramedullary, intramuscular, intralumbar, intrathecal, subarachnoid, intraventricular, intracapsular, intravenous, intralymphatical, intraocular and intraperitoneal routes, and wherein the administration method is injection, transfusion, or implantation.

Embodiment II-66. The method of embodiment II-64 or II-65, wherein the subject is selected from the group consisting of mouse, rat, pig, and non-human primate.

Embodiment II-67. The method of embodiment II-64 or II-65, wherein the subject is a human.

Embodiment II-68. A method of making a rAAV, comprising:
a. providing a population of packaging cells; and
b. transfecting the population of cells with:
   i) a vector comprising the transgene of any one of embodiments II-1 to II-39;
   ii) a vector comprising an Assembly-Activating Protein (AAP) gene; and
   iii) a vector comprising rep and cap genomes.

Embodiment II-69. The method of embodiment II-68, wherein the packaging cell is selected from the group consisting of BHK cells, HEK293 cells, HEK293T cells, NSO cells, SP2/0 cells, YO myeloma cells, P3X63 mouse myeloma cells, PER cells, PER.C6 cells, hybridoma cells, NIH3T3 cells, COS cells, HeLa cells, and CHO cells.

Embodiment II-70. The method of embodiment II-68 or II-69, the method further comprising recovering the rAAV.

Embodiment II-71. The method of any one of embodiments II-68 to II-70, wherein the component sequences of the transgene are encompassed in a single recombinant adeno-associated virus particle.

Embodiment II-72. A composition of a recombinant adeno-associated virus of any one of embodiments II-35 to II-56, for use in the manufacture of a medicament for the treatment of a disease in a human in need thereof.

Embodiment II-73. A kit comprising the rAAV of any one of embodiment II-35 to II-56 and a suitable container.

Embodiment II-74. The kit of embodiment II-73, comprising a pharmaceutically acceptable carrier, diluent, buffer, or excipient.

### EXAMPLES

### Example 1: Small Class 2, Type V CRISPR Proteins can Edit the Genome when Expressed from an AAV Episome In Vitro

Experiments were conducted to demonstrate that small Class 2, Type V CRISPR proteins can edit a genome when expressed from an AAV plasmid or an AAV vector in vitro.

### Materials and Methods

The AAV transgene between the ITRs was broken into different parts, which consisted of the therapeutic cargo and accessory elements relevant to expression of the therapeutic cargo in mammalian cells. AAV vectorology consisted of identifying a parts list and subsequently designing, building, and testing vectors in both plasmid and AAV form in mammalian cells. A schematic of a representative AAV transgene and one configuration of its components is shown in FIG. 1.

In this first example, three plasmids were constructed (construct 1, construct 2, and construct 3; see Table 45 for component sequences), where the only difference in the plasmid sequence between the ITRs was in the affinity tag region.

### Cloning and QC:

AAV vectors were cloned using a 4-part Golden Gate Assembly consisting of a pre-digested AAV backbone, small CRISPR protein-encoding DNA, and flanking 5′ and 3′ DNA sequences. 5′ sequences contained enhancer, protein promoter and N-terminal NLS, while 3′ sequences contained C-terminal NLS, Woodchuck Hepatitis Virus (WHV) Posttranscriptional Regulatory Element (WPRE), poly(A) signal, RNA promoter and guide RNA containing spacer 12.7, targeting tdTomato (DNA sequence: CTGCATTCTAGTTGTGGTTT (SEQ ID NO: 4049)). 5′ and 3′ parts were ordered as gene fragments, PCR-amplified, and assembled into AAV vectors through cyclical Golden Gate reactions using T4 Ligase and BbsI.

Assembled AAV vectors were then transformed into chemically-competent E. coli (Stbl3s). Transformed cells were recovered for 1 hour in a 37° C. shaking incubator, plated on Kanamycin LB-Agar plates and allowed to grow at 37° C. for 12-16 hours. Colony PCR was performed to determine clones that contained full transgenes. Correct clones were inoculated in 50 mL of LB media with kanamycin and grown overnight. Plasmids were then midiprepped the following day and sequence-verified. To assess the quality of plasmid preparations, constructs were processed in restriction digests with XmaI (which cuts in each of the ITRs) and XhoI (which cuts once in the AAV genome). Digests and uncut constructs were then run on a 1% agarose gel and imaged on a ChemiDoc™. If the plasmid was >90% supercoiled, the correct size, and the ITRs were intact, the construct was tested via nucleofection and/or transduction.

### Method for Plasmid Nucleofection:

Plasmids containing the AAV genome were transfected in a mouse immortalized neural progenitor cell line isolated from the Ai9-tdTomato mouse neuroprogenitor cells (tdTomato mNPCs) using the Lonza P3 Primary Cell 96-well Nucleofector Kit. Briefly, Ai9 is a Cre reporter tool strain designed to have a loxP flanked STOP cassette preventing the transcription of a CAG promoter-driven tdTomato marker. Ai9 mice, or Ai9 mNPCs, express tdTomato following Cre-mediated recombination to remove the STOP cassette. Sequence-validated plasmids were diluted to concentrations of 200 ng/μl, 100 ng/μl, 50 ng/μL and 25 ng/μL, and 5 μL of each (1000 ng, 500 ng, 250 ng and 125 ng) were added to P3 solution containing 200,000 tdTomato mNPCs. The combined solution was nucleofected using a Lonza 4D Nucleofector System following program EH-100. Following nucleofection, the solution was quenched with pre-equilibrated mNPC medium (DMEM/F12 with GlutaMax™, 10 mM HEPES, 1×MEM Non-Essential Amino Acids, 1× penicillin/streptomycin, 1:1000 2-mercaptoethanol, 1×B-27 supplement, minus vitamin A, 1×N2 with supplemented growth factors bFGF and EGF (20 ng/mL final concentration). The solution was then aliquoted in triplicate (approx. 67,000 cells per well) in a 96-well plate coated with PLF (1× Poly-DL-ornithine hydrobromide, 10 mg/mL in sterile diH20, 1× laminin, and 1× fibronectin). 48 hours after transfection, treated cells were replenished with fresh mNPC media containing growth factors. 5 days after transfection, tdTomato mNPCs were lifted and activity was assessed by fluorescence activated cell sorting (FACS).

AAV production:

Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free OPTI-MEM® media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15 M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then centrifuged at 4000 rpm for 20 minutes at 4° C. to resuspend the PEG precipitated AAV (pellet) with cell debris-free crude lysate (supernatant), and then clarified further using a 0.45 μM filter.

To determine the viral genome titer, 1 μL from crude lysate containing viruses was digested with DNase and proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region or a 62 nucleotide-fragment located in the AAV2-ITR. Ten-fold serial dilutions of an AAV ITR plasmid was used as reference standards to calculate the titer (viral genome (vg)/mL) of viral samples.

### AAV Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from −1.0e+6 to 1.0e+4 vg/cell. Final volumes of 50 μL of AAV vectors diluted in pre-equilibrated mNPC medium supplemented with bFGF/EGF growth factors (20 ng/ml final concentration) were applied to each well. 48 hours post-transfection, a complete media change was performed with fresh media supplemented with growth factors. Editing activity (tdT+ cell quantification) was assessed by FACS 5 days post-transfection.

### Method for Assessing Activity by FACS:

Five days after transfection, treated tdTomato mNPCs in 96-well plates were washed with dPBS and treated with 50 μL TrypLE for 15 minutes. Following cell dissociation, treated wells were quenched with media containing DMEM, 10% FBS and 1× penicillin/streptomycin. Resuspended cells were transferred to round-bottom 96-well plates and centrifuged for 5 min at 1000×g. Cell pellets were then resuspended with dPBS containing 1×DAPI, and plates were loaded into an Attune™ NxT Flow Cytometer Autosampler. The Attune™ NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

### Results

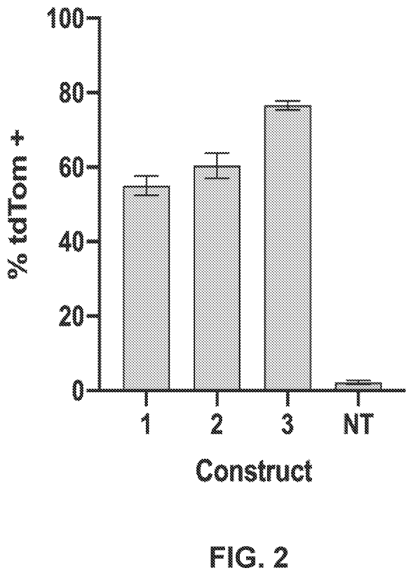

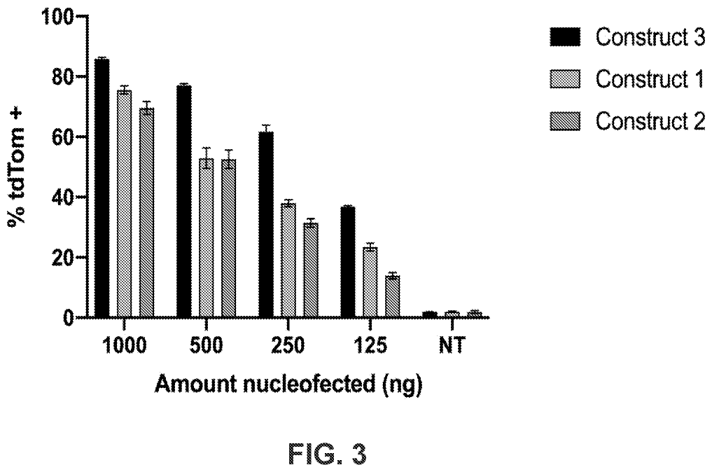

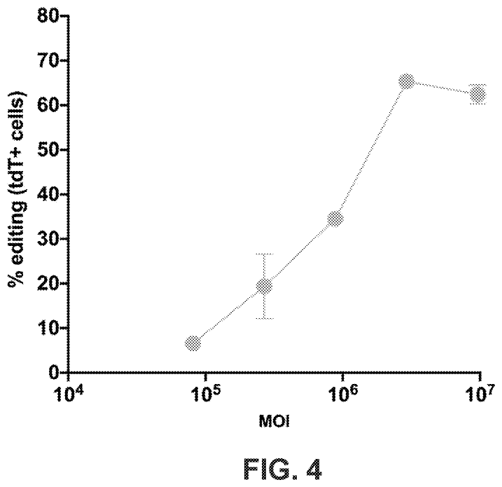

The results in the graph in FIG. 2 shows that CasX variant 491 and guide variant 174 with spacer 12.7 targeting the tdTomato stop cassette, when delivered by nucleofection of an AAV transgene plasmid, was able to edit the target stop cassette in mNPCs (measured by percentage of cells that are tdTom+ by FACS). Among the vectors tested, CasX 491.174 delivered in construct 3 (with 80% tdTomato+cells) outperformed the others. FIG. 3 shows that all three vectors tested achieved editing at the tdTomato locus in a dose-dependent manner. FIG. 4 shows results of editing using construct 3 in an AAV vector, which demonstrated a dose-dependent response, achieving a high degree of editing.

The experiments demonstrate that small Class 2, Type V CRISPR proteins (such as CasX) and targeted guides can edit the genome when expressed from an AAV transgene plasmid or episome in vitro.

### Example 2: Packaging of Small Class 2, Type V CRISPR Systems within an AAV Vector

Experiments were conducted to demonstrate that systems of small Class 2, Type V CRISPR proteins such as CasX and gRNA can be encoded and efficiently packaged within a single AAV vector.

### Materials and Methods

For this experiment, AAV vectors were generated with transgenes packaging CasX variant 438, gRNA scaffold 174 and spacer 12.7 using the methods for AAV production, purification and characterization, as described in Example 1. For characterization, AAV viral genomes were titered by qPCR, and the empty-full ratio was quantified using scanning transmission electron microscopy (STEM). The AAV were negatively stained with 1% uranyl acetate and visualized. Empty particles were identified by presence of a dark electron dense circle at the center of the capsid.

### Results

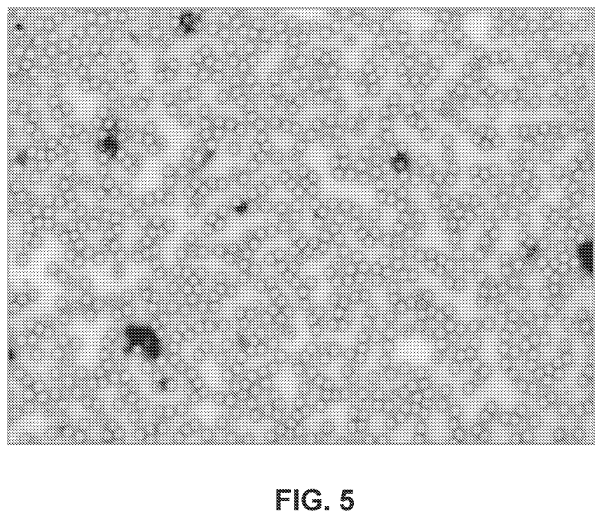

The genomic DNA titers (by qPCR) for the AAV preparation was measured to be 6e12 vg/mL, generated from 1 L of HEK293T cell culture. FIG. 5 is an image from a scanning transmission electron microscopy (STEM) micrograph showing that an estimated 90% of the particles in this AAV formulation contained viral genomes, i.e., loaded with the CRISPR cargo. These results demonstrate that sequences encoding CasX variant proteins and gRNA can be efficiently packaged in an AAV vector, resulting in high titers and high packaging efficiency.

### Example 3: In Vivo Editing of a Genome with Small Class 2, Type V CRISPR Proteins Expressed from an AAV Episome

Experiments were conducted to demonstrate that small Class 2, Type V CRISPR proteins, such as CasX, are capable of being delivered by AAV and can edit the genome when expressed from an AAV episome in vivo.

### Materials and Methods

For this experiment, AAV vectors were generated using the methods for AAV production, purification and characterization, as described in Example 1.

### In Vivo AAV Administration and Tissue Processing

P0-P1 pups from Ai9 mice were injected with AAV with a transgene encoding CasX variant 491 and guide variant 174 with spacer 12.7. Briefly, mice were cryo-anesthetized and 1-2 μL of AAV vector (˜1e11 viral genomes (vg)) was unilaterally injected into the intracerebroventricular (ICV) space using a Hamilton syringe (10 μL, Model 1701 RN SYR Cat No: 7653-01) fitted with a 33-gauge needle (small hub RN NDL—custom length 0.5 inches, point 4 (45 degrees)). Post-injection, pups were recovered on a warm heating pad before being returned to their cages. 1 month after ICV injections, animals were terminally anesthetized with an intraperitoneal injection of ketamine/xylazine, and perfused transcardially with saline and fixative (4% paraformaldehyde). Brains were dissected and further post-fixed in 4% PFA, followed by infiltration with 30% sucrose solution, and embedding in OCT compound. OCT-embedded brains were coronally sectioned using a cryostat. Sections were then mounted on slides, counter-stained with DAPI to label cell nuclei, cover slipped and imaged on a fluorescence microscope. Images were processed using ImageJ software and editing levels were quantified by counting the number of tdTom+ cells as a percentage of DAPI-labeled nuclei.

In a subsequent experiment to assess editing in peripheral tissues, particularly in the liver and in the heart, P0-P1 pups from Ai9 mice were cryo-anesthetized and were intravenously injected with ˜1e12 viral genomes (vg) of the same AAV construct in a 40 μL volume. Post-injection, pups were recovered on a warm heating pad before being returned to their cages. 1-month post-administration, animals were terminally anesthetized and heart and liver tissues were necropsied and processed as described above.

### Results

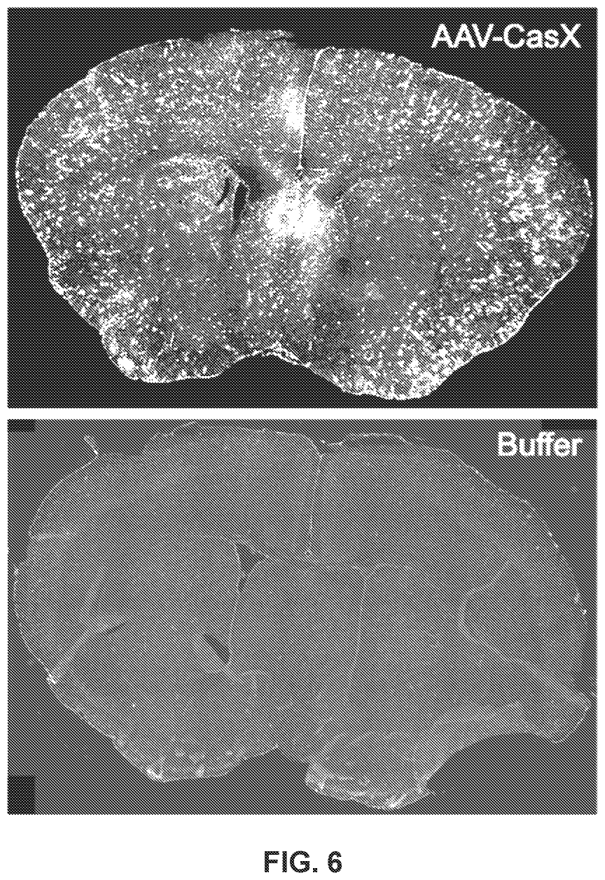

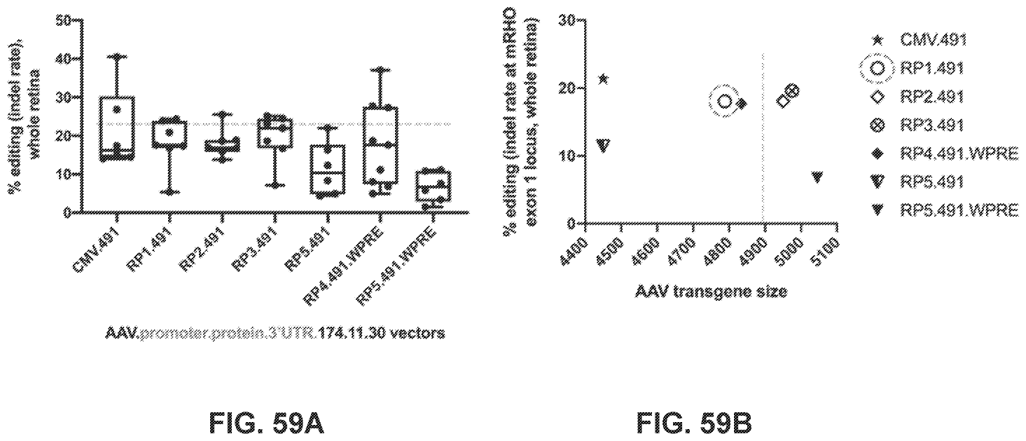

FIG. 6 provides comparative immunohistochemistry (IHC) images of brain tissue processed from an Ai9 mouse that received an ICV injection of AAV packaging CasX variant 491 and guide scaffold 174 with spacer 12.7. The tissue was stained with 4′,6-diamidino-2-phenylindole. The signal from cells in the tdTom channel indicates that the tdTom locus within these cells was successfully edited. The tdTom+ cells (in white) are distributed evenly across all regions of the brain, indicating that ICV-administered AAVs packaging the encoded CasX, guide and spacer were able to reach and edit these cells (top panel) as compared to a non-targeting control (bottom panel). The FIG. 6 images are representative of those obtained from 3 mice for each group. Additionally, the results presented in FIG. 59A (liver) and 59B (heart) demonstrate that the AAV were able to distribute within the liver and the heart (edited cells in white) and edit the genome when expressed from single AAV episomes in vivo.

The results demonstrate that that AAV encoding small CRISPR proteins (such as CasX) and a targeting guide can distribute within the tissues, when delivered either locally (brain) or systemically, and edit the target genome when expressed from single AAV episomes in vivo.

### Example 4: Small CRISPR Protein Potency is Enhanced by AAV Vector Protein Promoter Choice

Experiments were conducted to demonstrate that small CRISPR protein expression and editing can be enhanced by utilizing different promoters in an AAV construct for the encoded protein. Cargo space in the AAV transgene can be maximized with the use of short promoters in combination with small CRISPR proteins such as CasX. Additionally, experiments were conducted to demonstrate that expression can be enhanced with the use of promoters that would otherwise be too long to be efficiently packaged in AAV vector, if they were combined with larger CRISPR proteins, such as Cas9. The use of long, cell-type-specific promoters to enhance small CRISPR proteins is an advantage to the AAV system described herein, and not possible in traditional CRISPR systems due to the size of traditional CRISPR proteins.

### Materials and Methods

Cloning and QC were conducted as described in Example 1. Promoter variants (Table 7) were cloned upstream of CasX protein in an AAV-cis plasmid. The sequences of the additional components of the AAV constructs, with the exception of sequences encoding the CasX (Table 26) and the one or more gRNA (Tables 23 and 24), are listed in Table 45.

TABLE 7
Promoter variant sequences

| | | | Promoter |
|---|---|---|---|
| SEQ | Construct | Promoter | length |
| ID NO: | ID | based on | (bp) |
| 3532 | 1, 2, 3, 7, 44 | CMV | 584 |
| 3533 | 4 | UbC | 400 |
| 3534 | 5 | EFS | 234 |
| 3535 | 6 | CMV-s | 335 |
| 3536 | 8 | CMVd1 | 100 |
| 3537 | 9 | CMVd2 | 52 |
| 3538 | 10 | miniCMV | 39 |
| 3539 | 11, 26 | HSVTK | 146 |
| 3540 | 12 | miniTK | 63 |
| 3541 | 13 | miniIL2 | 114 |
| 3542 | 14 | GRP94 | 710 |
| 3543 | 15 | Supercore 1 | 81 |
| 3544 | 16 | Supercore 2 | 81 |
| 3545 | 17 | Supercore 3 | 81 |
| 3546 | 18 | Mecp2 | 229 |
| 3547 | 19 | CMVmini | 68 |
| 3548 | 20 | CMVmini2 | 65 |
| 3549 | 21 | miniCMVIE | 39 |
| 3550 | 22 | adML | 81 |
| 3551 | 23 | hepB | 107 |
| 3552 | 54 | RSV | 227 |
| 3553 | 55 | hSyn | 448 |
| 3554 | 56 | SV40 | 330 |
| 3555 | 57 | hPGK | 551 |
| 3556 | 58 | Jet | 164 |
| 3557 | 59 | Jet + UsP intron | 326 |
| 3558 | 60 | hRLP30 | 325 |
| 3559 | 61 | hRPS18 | 243 |
| 3560 | 62 | CBA | 493 |
| 3561 | 63 | CBH | 565 |
| 3562 | 64 | CMV core | 204 |

### Method for Plasmid Nucleofection:

Immortalized neural progenitor cells were nucleofected as described in Example 1. Sequence-validated plasmids were diluted to concentrations of 200 ng/ul, 100 ng/ul, 50 ng/μL and 25 ng/μL, and 5 μL of each (1000 ng, 500 ng, 250 ng and 125 ng) were added to P3 solution containing 200,000 tdTomato mNPCs.

AAV viral production and QC, and AAV transduction and editing level assessment in mNPTC-tdT cells by FACS were conducted as described in Example 1.

### Results

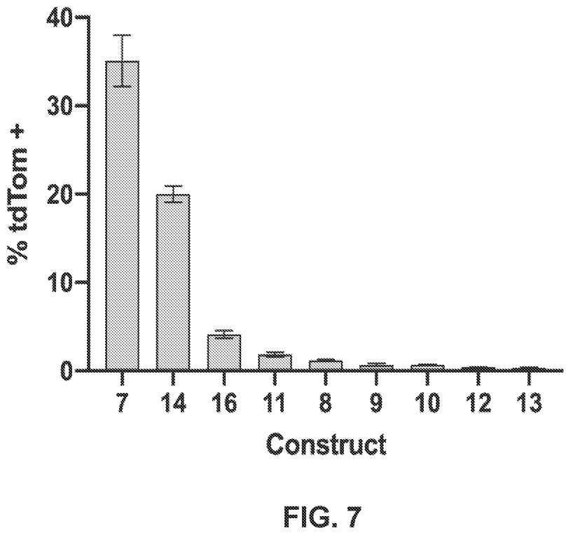

The results of FIG. 7 demonstrate that several different promoters with CasX protein 438, scaffold variant 174 and spacer targeting the tdTomato stop cassette (spacer 12.7, with sequence CTGCATTCTAGTTGTGGTTT (SEQ ID NO: 4049)), when delivered by nucleofection of AAV transgene plasmid, were able to edit the target stop cassette in mNPCs at a dose of 1000 ng. These promoters ranged in length from over 700 nucleotides to as short as 81 nucleotides (Table 7). Among the promoters tested, construct 7 and 14 showed considerable editing potency.

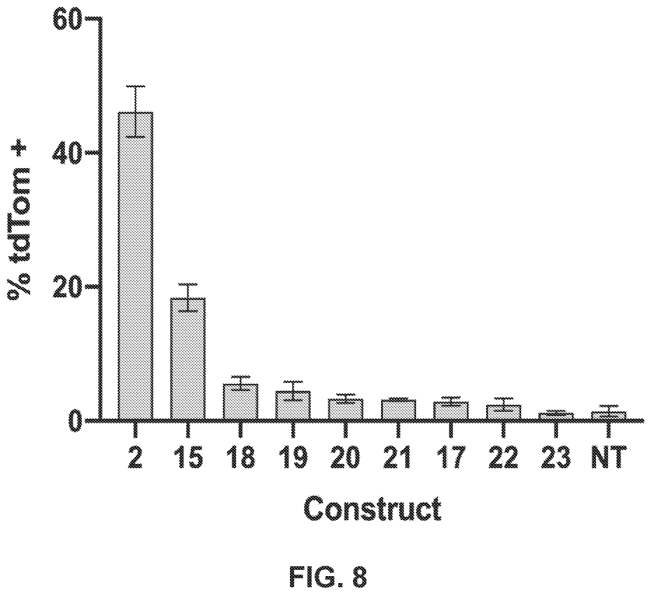

The results of FIG. 8 demonstrate that several short promoters combined with CasX variant 491, scaffold variant 174 and spacer 12.7, when delivered by nucleofection of AAV transgene plasmid, edit the target stop cassette in mNPCs at a dose of 500 ng. Other than construct 2, which had a promoter of 584 nucleotides, all constructs had promoters that were less than 250 nucleotides in length. Among the protein promoters tested, construct 15 showed considerable editing potency, especially given its short length (81 nucleotides).

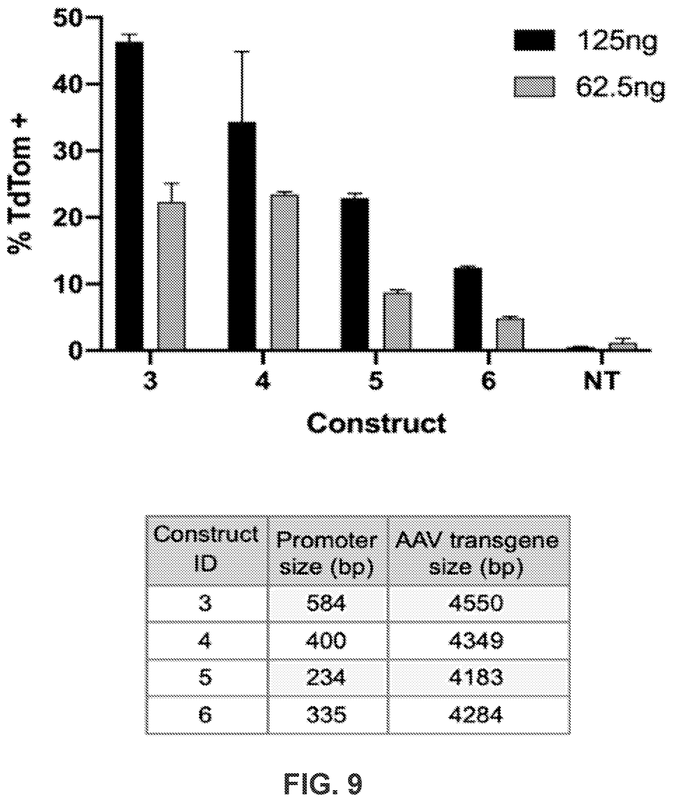

The results of FIG. 9 demonstrate that four promoters with CasX variant 491 and scaffold variant 174 with spacer 12.7, when delivered by nucleofection of AAV transgene plasmid, edit the target stop cassette in mNPCs at doses of 125 ng and 62.5 ng. Constructs 4, 5 and 6 have promoter lengths less than or equal to 400 nucleotides, and thus may maximize editing potency while minimizing AAV cargo capacity.

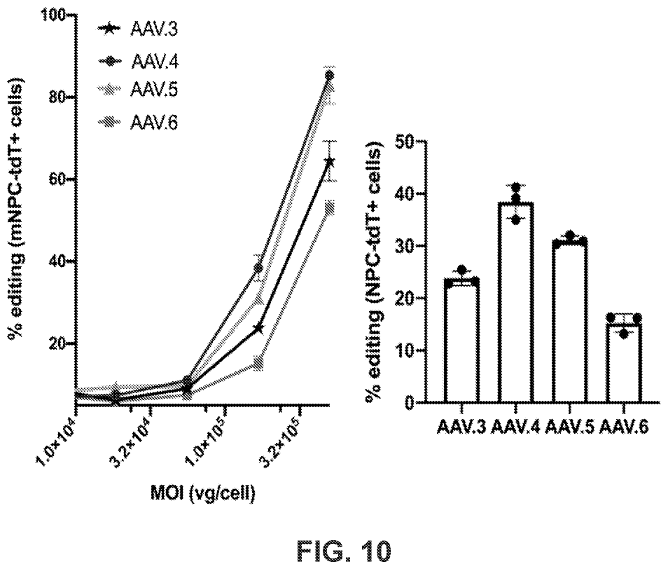

The results shown in FIG. 10 demonstrate that use of four promoter variants in the AAV also result in robust editing. Briefly, AAVs (AAV.3, AAV.4, AAV.5 and AAV.6) were generated with transgene constructs 3-6, respectively. Each construct showed dose-dependent editing at the target locus (FIG. 10, left panel). At an MOI of 2e5, AAV.4 showed editing at 38%±3% at the target locus, outperforming the other constructs (FIG. 10, right panel).

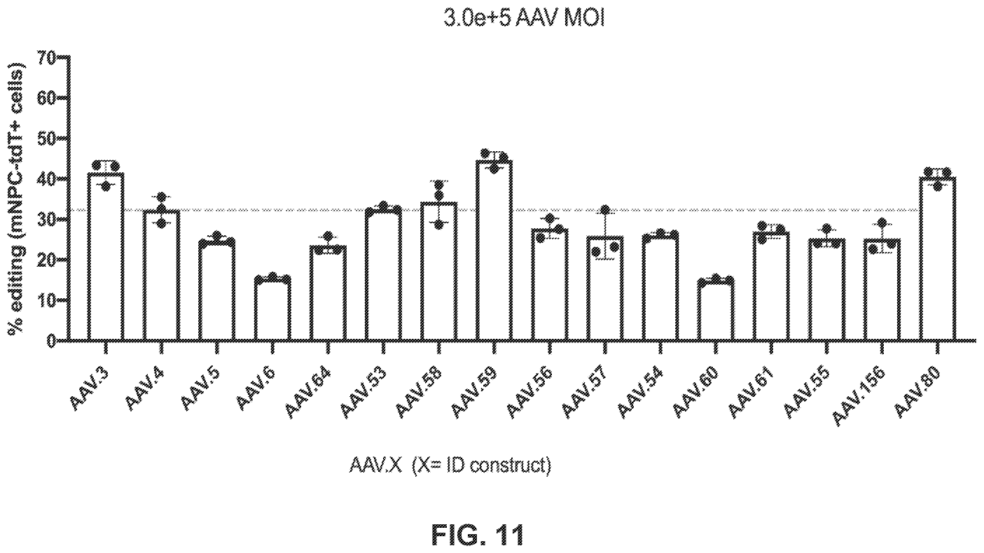

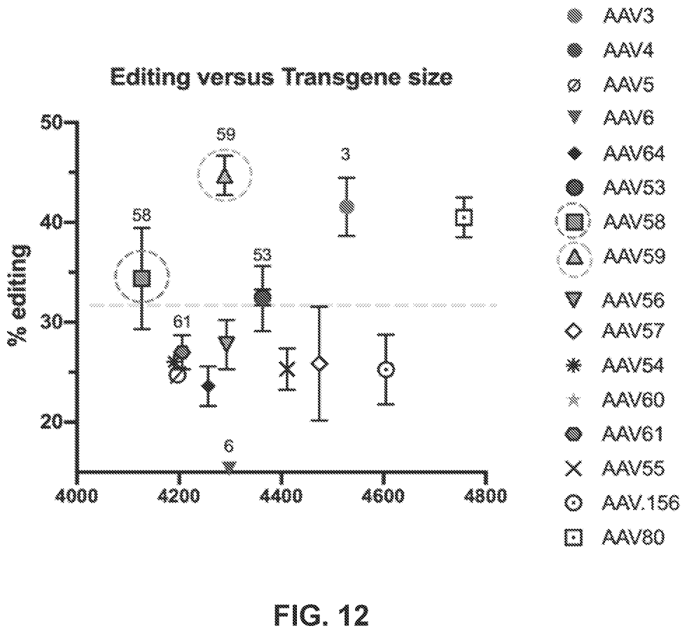

In the experiments performed for the results portrayed in FIG. 11, several new protein promoters were compared against the top 4 protein promoter variants identified previously (AAV.3, AAV.4, AAV.5 and AAV.6). Briefly, AAVs were generated with corresponding transgene constructs and transduced in tdTomato mNPCs. At an MOI of 3e5 at 5 days after transduction, multiple promoters displayed improved editing (FIG. 11). In particular, constructs 58 and 59 had editing activity above 30% while minimizing transgene size (FIG. 12). Construct 58 and 59 contained promoters that are 420 and 258 bp smaller, respectively, than construct 3, yet resulted in similar or improved editing of the target locus. In particular, inclusion of an intron in the promoter of construct 59 led to increased editing compared to construct 58, which lacked the intron, demonstrating that the inclusion of introns in the AAV construct promoters is beneficial.

The results demonstrate that expression of small CRISPR proteins (such as CasX) can be enhanced by utilizing long promoters that would otherwise be unusable in AAV constructs with traditional CRISPR proteins due to the size constraints of the AAV genome. Furthermore, combining short promoters with small CRISPR proteins (such as CasX) allows for significant reductions in AAV transgene cargo capacity without compromising expression efficiency. This conservation of space allows for the inclusion of additional accessory elements, such as enhancers and regulatory elements in the transgene, which would enable increased editing potential. Example 35 further demonstrates and evaluates various protein promoters on CasX protein editing activity in a cell-based assay.

### Example 5: Potency of Small CRISPR Systems is Enhanced by AAV RNA Promoter Choice

Experiments were conducted to demonstrate that the editing potency of small CRISPR systems, such as CasX, can be enhanced if certain promoters are chosen for expression of the guide RNA, which recognizes target DNA for editing, in an AAV vector. By using RNA promoters with different strengths, guide RNA expression can be modulated, which affects editing potency. The AAV platform based on the CasX system provides enough cargo space in the AAV to include at least 2 independent promoters for the expression of two incorporated guide RNAs. By combining different promoters, expression of multiple guide RNAs can be tuned within a single AAV transgene. Engineering shorter versions of RNA promoters that still retain editing potency also results in increased space in the vector for the inclusion of other accessory elements in the AAV transgene.

### Materials and Methods

The methods of Example 1 were used for cloning and quality control of the constructs, as well as for plasmid nucleofection and AAV production, transduction, and FACS analyses. The sequences of the Pol III promoters are presented in Table 8. The sequences of the additional components of the AAV constructs, with the exception of sequences encoding the CasX (Table 26) and the one or more gRNA (Tables 23 and 24), are listed in Table 45.

TABLE 8
Sequences of Pol III promoters.

| | | | Promoter |
|---|---|---|---|
| SEQ | AAV | Pol III | length |
| ID NO: | construct ID | promoter | (bp) |
| 3563 | 3, 53, 157 | hU6 isoform 1 | 241 |
| 3564 | 32, 158 | H1 | 215 |
| 3565 | 33 | 7SK | 267 |
| 3566 | 85/89 | hU6 variant 1 | 103 |
| 3567 | 86 | hU6 variant 2 | 38 |
| 3568 | 87 | hU6 variant 3 | 67 |
| 3569 | 88 | hU6 variant 4 | 79 |
| 3570 | 90 | hU6 variant 5 | 111 |
| 3571 | 91 | hU6 variant 6 | 127 |
| 3572 | 92 | hU6 variant 7 | 123 |
| 3573 | 93 | hU6 variant 8 | 143 |
| 3574 | 94 | hU6 variant 9 | 131 |
| 3575 | 95 | hU6 variant 10 | 159 |
| 3576 | 96 | hU6 variant 11 | 103 |
| 3577 | 97 | hU6 variant 12 | 111 |
| 3578 | 98 | hU6 variant 13 | 127 |
| 3579 | 99 | hU6 variant 14 | 103 |
| 3580 | 100 | hU6 variant 15 | 131 |
| 3581 | 101 | hU6 variant 16 | 159 |
| 3582 | 102 | hU6 variant 17 | 128 |
| 3583 | 159 | H1 core | 91 |
| 3584 | 160 | H1 core + 7SK hybrid 1 | 92 |
| 3585 | 161 | H1 core + 7SK hybrid 2 | 92 |
| 3586 | 162 | H1 core + 7SK hybrid 3 | 91 |
| 3587 | 163 | H1 core + 7SK hybrid 4 | 91 |
| 3588 | 164 | H1 core + 7SK hybrid 5 | 92 |
| 3589 | 165 | H1 core + 7SK hybrid 6 | 91 |
| 3590 | 166 | H1 core + 7SK hybrid 7 | 91 |
| 3591 | 167 | H1 core + 7SK hybrid 8 | 91 |
| 3592 | 168 | H1 core + 7SK hybrid 9 | 92 |
| 3593 | 169 | H1 core + U6 hybrid 1 | 91 |
| 3594 | 170 | H1 core + U6 hybrid 2 | 94 |
| 3595 | 171 | H1 core + 7SK + | 92 |
| | | U6 hybrid 1 | |
| 3596 | 172 | H1 core + U6 hybrid 3 | 90 |
| 3597 | 173 | H1 core + 7SK + | 94 |
| | | U6 hybrid 2 | |
| 3598 | 174 | H1 core + 7SK + | 94 |
| | | U6 hybrid 3 | |
| 3599 | — | hU6 isoform 2 | 249 |
| 3600 | — | hU6 isoform 3 | 249 |
| 3601 | — | hU6 isoform 4 | 249 |
| 3602 | — | hU6 isoform 5 | 249 |
| 3603 | — | mU6 | 304 |
| 3604 | — | mU6 isoform | 314 |
| 3605 | — | gorilla U6 | 241 |
| 3606 | — | Saimiri U6 | 229 |
| 3607 | — | Macaca U6 | 240 |
| 3608 | — | Papio U6 | 240 |
| 3609 | — | Rhinopithecus U6 | 240 |
| 3610 | — | Mini Gorilla U6 | 103 |
| 3611 | — | Mini Saimiri U6 | 102 |
| 3612 | — | Mini Macaca U6 | 102 |
| 3613 | — | Mini Papio U6 | 102 |
| 3614 | — | Mini Rhinopithecus U6 | 102 |

### Results

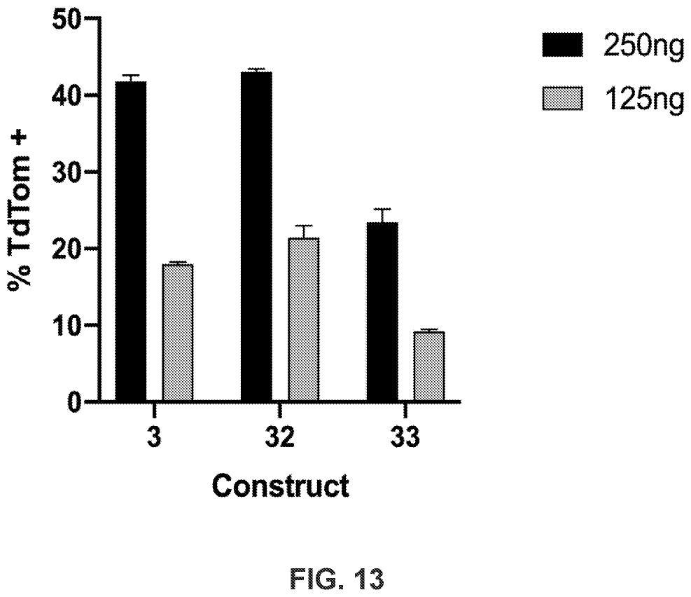

The results portrayed in FIG. 13 demonstrate that AAV vectors using three distinct RNA promoters, in combination with CasX protein 491, scaffold variant 174 and spacer 12.7, when delivered by nucleofection of the AAV transgene plasmid, edit the target stop cassette in mNPCs at doses of 250 ng and 125 ng. Construct 3 (U6 promoter) and construct 32 (H1 promoter) have similar activity, editing at the target locus with 42% efficiency. Construct 33 shows ˜56% of the activity of constructs 3 and 32.

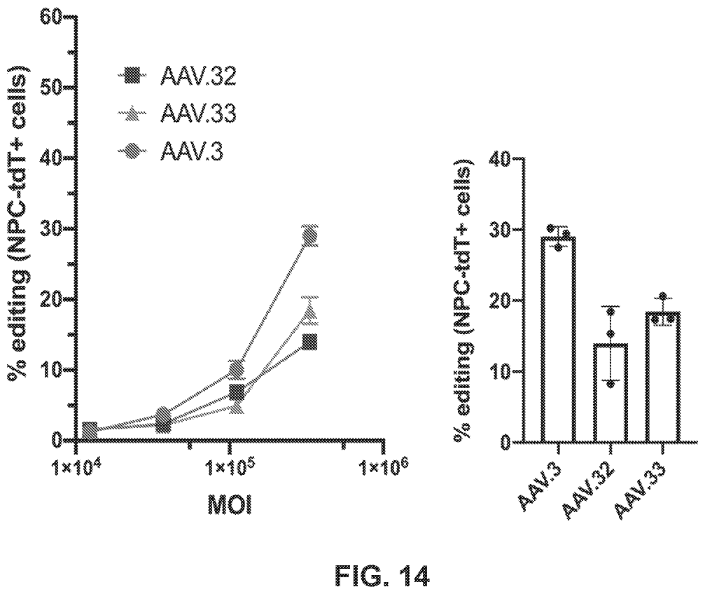

The results portrayed in FIG. 14 demonstrate that the same three distinct promoters, in combination with CasX protein 491, scaffold variant 174 and spacer 12.7, when delivered as AAV, edit the target stop cassette in mNPCs. AAV.3, AAV.32, AAV.33 were generated with transgene constructs 3, 32 and 33 respectively. Each vector displayed dose-dependent editing at the target locus (FIG. 14, left panel). At an MOI of 3e5, AAV.32 and AAV.33 had 50-60% of the potency of AAV.3 (FIG. 14, right panel).

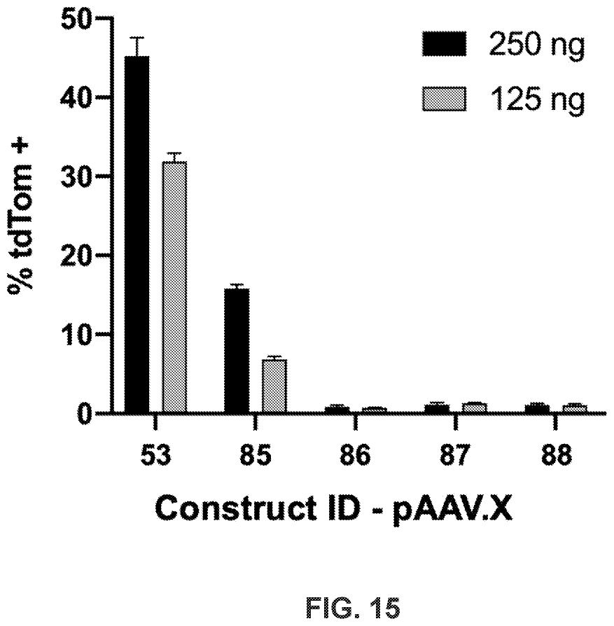

The results shown in FIG. 15 demonstrate that constructs having one of four different truncations of the U6 promoter, in combination with CasX protein 491, scaffold variant 174 and spacer 12.7, when delivered by nucleofection of the AAV transgene plasmid, were each able to edit the target stop cassette at different levels in mNPCs at doses of 250 ng and 125 ng. Construct 85 (hU6 variant 1) had 33% of the potency of the base construct 53 (hU6), while construct 86 (hU6 variant 2), construct 87 (hU6 variant 3) and construct 88 (hU6 variant 4) did not show any editing and were comparable to a non-targeting control.

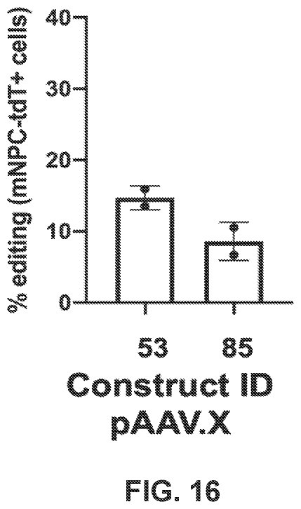

FIG. 16 presents results of an experiment comparing editing in mNPCs between AAV generated with base construct 53 (hU6 promoter) to AAV generated with construct 85 (hU6 variant 1). When delivered as AAV, AAV.85 was able to edit at 7% compared to 15% for AAV.53 at an MOI of 3e5, consistent with the results from FIG. 15.

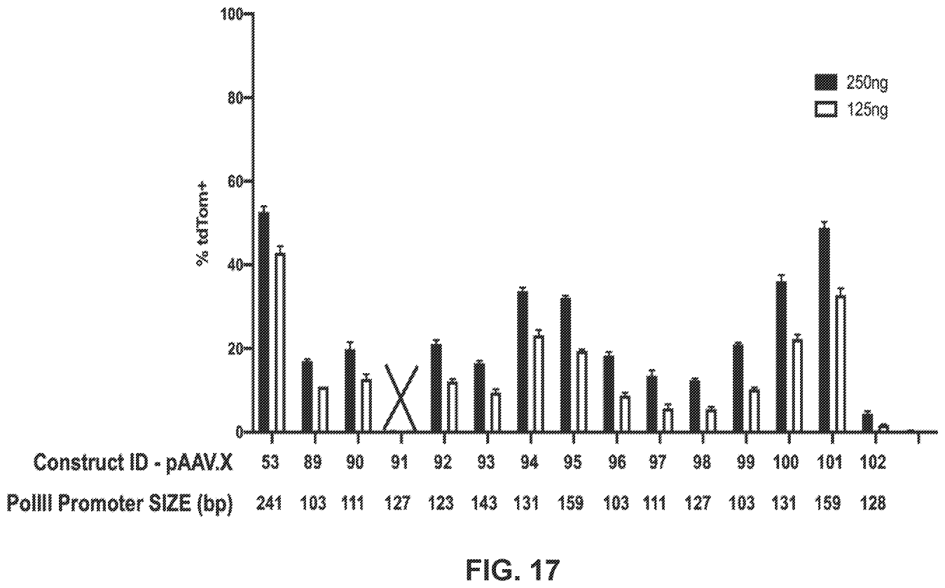

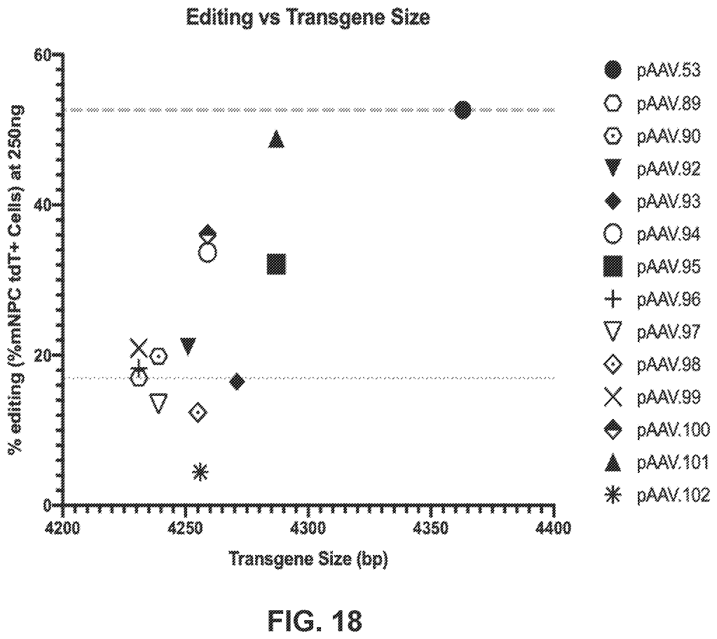

The results of FIG. 17 demonstrate that constructs with engineered U6 promoters were able to edit the target stop cassette at different levels in mNPCs at doses of 250 ng and 125 ng. Engineered U6 promoters were designed to minimize the size of the promoter relative to the base U6 promoter. Construct AAV.53 carried the hU6 promoter, in combination with encoded CasX protein 491, scaffold variant 174 and spacer 12.7, and the constructs with the variant promoters carried the same CasX, scaffold and spacer as AAV.53. Constructs were delivered to mNPCs by nucleofection of AAV transgene plasmid, and were able to edit the target stop cassette at different levels in mNPCs at doses of 250 ng and 125 ng. One cluster of constructs (AAV.89 (hU6 variant 1), 90 (hU6 variant 5), 92 (hU6 variant 7), 93 (hU6 variant 8), 96 (hU6 variant 11), 97 (hU6 variant 12), 98 (hU6 variant 13), and 99 (hU6 variant 14)) all edited in the range of 15-20%, compared to 55% for construct AAV53. Other Pol III variants (constructs AAV94 (hU6 variant 9), 95 (hU6 variant 10) and 100 (hU6 variant 15)) all exhibited higher levels of editing at around 32% editing while construct 101 resulted in 48% editing. These promoters are all smaller than the Pol III promoter in the base construct AAV53, as shown in the scatterplot of FIG. 18, depicting transgene size of all AAV variants tested having engineered U6 RNA promoters on the X-axis vs. percent of mNPCs edited on the Y-axis.

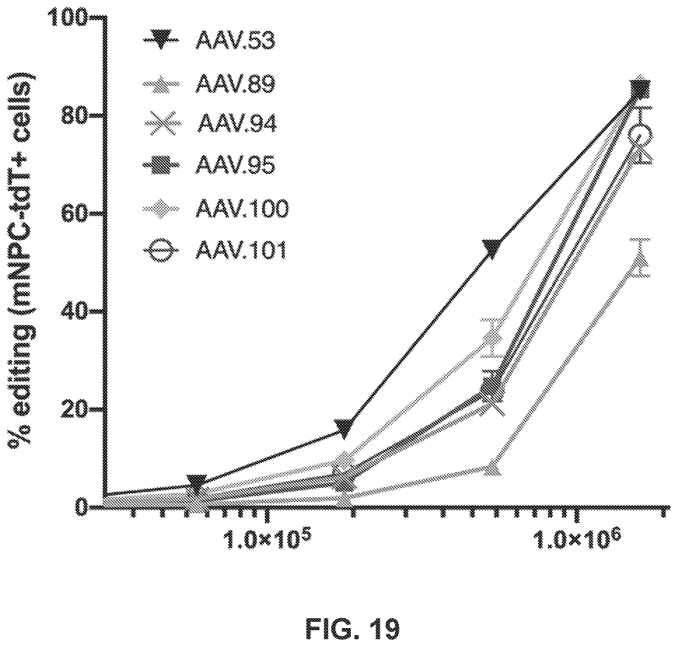

The results depicted in FIG. 19 show that constructs with engineered U6 promoters with CasX 491, scaffold variant 174 and spacer 12.7, when delivered as AAV, were able to edit the target stop cassette in mNPCs in a dose-dependent fashion. Variable rates of editing mediated by AAV with constructs AAV.94, AAV.95, AAV.100, and AAV.101 were seen, all editing at rates between the base construct AAV.53 and AAV.89, which has the same Pol III promoter as AAV.85 from FIGS. 15 and 16.

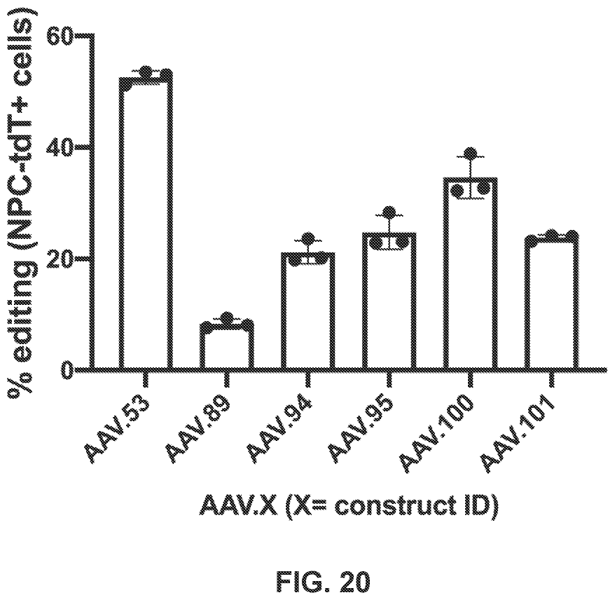

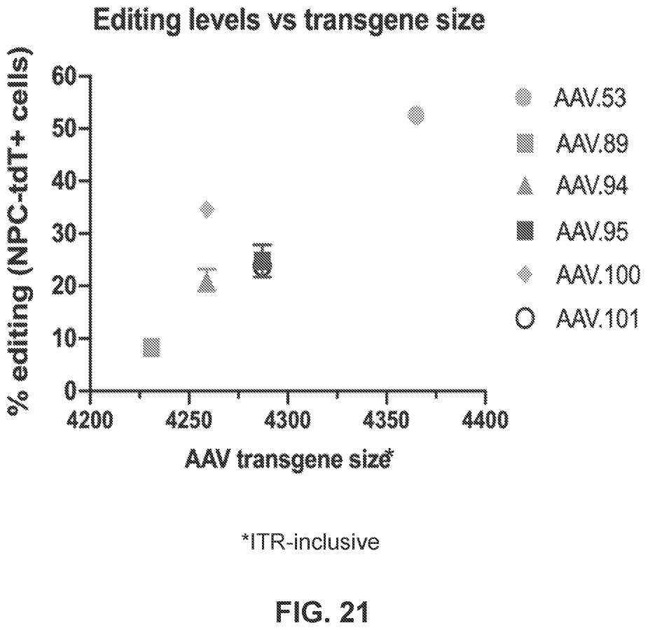

The results in FIG. 20 show that constructs with engineered U6 promoters with CasX protein 491, scaffold variant 174 and spacer 12.7, when delivered as AAV, were able to edit the target stop cassette in mNPCs. Variable rates of editing with AAV with constructs AAV.94, AAV.95, AAV.100, and AAV.101 were seen, all editing at rates between the base construct AAV.53 and AAV.89, which has the same Pol III promoter as AAV.85 from FIGS. 15 and 16. FIG. 21 shows the results as a scatterplot of editing versus transgene size.

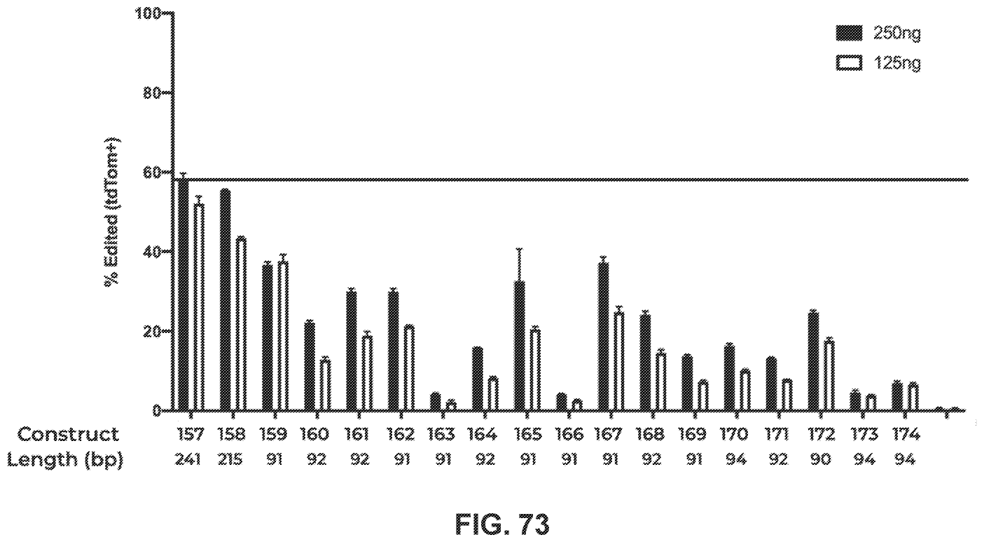

The results depicted in FIG. 73 demonstrate that AAV constructs with rationally engineered Pol III promoters, with sequences encoding for CasX protein 491, scaffold variant 174, and spacer 12.7, were able to edit the target tdTomato stop cassette at varying efficiencies when nucleofected as AAV transgene plasmids into mouse NPCs at doses 250 ng and 125 ng. Constructs 159 to 174 were designed to minimize the size of the promoter relative to the base U6 (construct ID 157) or H1 (construct ID 158) promoter, and constructs 160 to 174 were engineered as short, hybrid variants based on a core region of the H1 promoter (construct 159) with variations of domain swaps from 7SK and/or U6 promoters. FIG. 73 shows that most of these promoter variants, which are substantially shorter than the base U6 and H1 promoters, were able to function as Pol III promoters to drive sufficient gRNA transcription and editing at the tdTomato locus. Specifically, constructs 159, 161, 162, 165, and 167 were able to achieve at least 30% editing at the higher dose of 250 ng. These variants serve as promoter alternatives in AAV construct design that would permit significant reductions in AAV cargo capacity while driving adequate gRNA expression for targeted editing.

Additional RNA promoters can be identified via substitutions and deletions of the U6 promoter and mining for alternative guide RNA promoters from non-human species. To test these RNA promoters in a high-throughput manner, a screening assay is developed to test a library of U6 promotor sequences (SEQ ID NOS: 48-100, 513-566, 594-2100, and 4133-9256) containing all single bp substitutions and single-, double-, 5-, and 10-bp deletions of the human U6 promoter and alternative non-human primate RNA promoters. This library of sequences is synthesized as DNA oligos, amplified and cloned into a lentiviral construct containing different CasX variants, including CasX variants 491, 515, 593, 668, 672, 676, and 812, gRNA scaffold 235 with spacer 34.19, which edits the HBEGF locus and confers cell survival. Briefly, HBEGF is a receptor that mediates entry of diphtheria toxin, which, when added to the cells, inhibits translation and results in cell death. Targeting the HBEGF locus with CasX and HBEGF-targeting spacer should prevent toxin entry and allow cell survival. The resulting lentiviral library is used to transduce HEK293T cells, followed by selection at 2 ng/mL of toxin for 48 hours. After selection, genomic DNA (gDNA) is isolated and used to PCR an amplicon containing the U6 promoter in the surviving cells. These amplicons are sequenced, and frequencies are compared to the pre-selection library to identify U6 promoters that increase in frequency by resulting in more potent CasX:gRNA-mediated editing of the HBEGF locus. This screening assay may be repeated at higher doses, various timepoints, and different cell types to identify more active U6 promoters that induce greater CasX:gRNA-mediated editing. The results of these screens are expected to allow for a ranking of U6 promoters by fitness scores, many of which are anticipated to be better than the current set of lead molecules described in the preceding Examples. The U6 promoters that result in strong survival in all cell types across the doses utilized are prioritized for further characterization as elements in AAV vectors.

The results of these experiments demonstrate that expression of small CRISPR systems, such as CasX and gRNAs, can be modulated in various ways by utilizing alternative RNA promoters to express the gRNA. While most other CRISPR systems utilized in AAV do not have sufficient space in the transgene to include a separate promoter to express the gRNA, the CasX CRISPR system, and other systems with similarly small size, enable the use of multiple gRNA promoters of varying lengths within a single AAV transgene. These promoters can be used to differentially control expression and editing by the AAV transgene. The data also show that shorter versions of Pol III promoters can be engineered to retain their ability to facilitate transcription of functional guides. This increases the capacity of the AAV transgene to include additional promoters and/or accessory elements. Furthermore, adjusting other elements in the AAV transgene allows for the combination of multiple gRNA transcriptional units that could result in the following: 1) increased gRNA expression and thus CasX-mediated editing; or 2) driving the expression of more than one gRNA from a single AAV system, which would enable the ability to deliver CasX with a dual-gRNA system from a single AAV vector for targeted editing at different locations in the genome (further discussed in Example 9).

### Example 6: Choice of Poly(A) Signal Sequence Enhances Potency of AAV Vectors

Experiments were conducted to demonstrate that small CRISPR proteins, such as CasX, can be expressed from an AAV genome utilizing a variety of polyadenylation (poly(A)) signal sequences. Specifically, use of sequences encoding smaller CRISPR systems enable the inclusion of larger poly(A) signal sequences in the transgene of AAV vectors. In addition, experiments were conducted to demonstrate that the inclusion of shorter synthetic poly(A) signal sequences in the AAV constructs allows for further reductions in the AAV transgene cargo capacity.

### Materials and Methods

### AAV Plasmid Cloning:

Poly(A) signal sequences within the AAV genome were separated by restriction enzyme sites to allow for modular cloning. Polyadenylation sequences were ordered as gene fragments and cloned into vector restriction sites according to standard techniques.

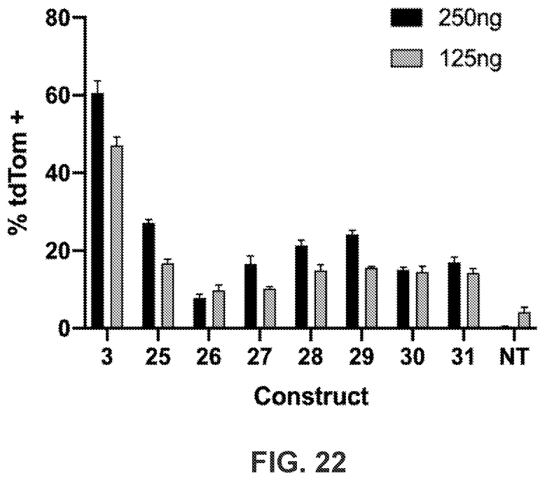

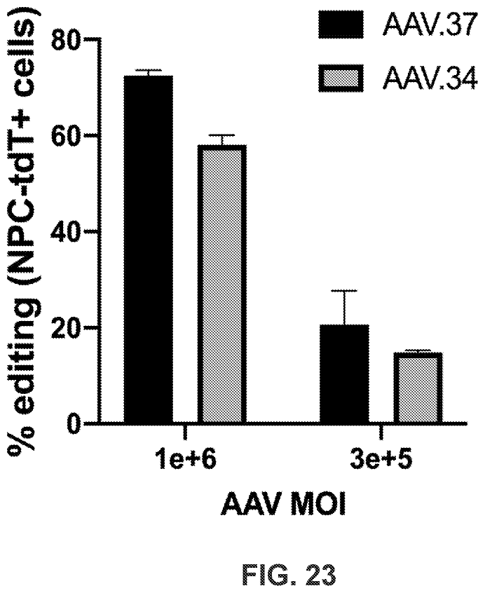

To generate the AAV plasmids assessed in the experiments resulting in the data presented in FIG. 22 and FIG. 23, the methods of Example 1 were used for cloning and quality control of the constructs, as well as for plasmid nucleofection and FACS analysis. The sequences of the poly(A) signals are presented in Table 9. The sequences of the additional components of AAV constructs, with the exception of sequences encoding the CasX (Table 26) and the one or more gRNA (Tables 23 and 24), are listed in Table 45.

TABLE 9
Poly(A) signal sequences

| AAV | | Poly(A) signal | SEQ |
|---|---|---|---|
| Construct ID | Poly(A) signal | sequence length (bp) | ID NO |
| 1, 3, 37, 232 | bGH | 208 | 3401 |
| 24, 227 | hGH | 623 | 3402 |
| 25, 228 | hGHshort | 477 | 3403 |
| 26, 231 | HSVTK | 49 | 3404 |
| 27, 221 | SynPolyA | 49 | 3405 |
| 28, 226 | SV40 | 122 | 3406 |
| 29 | SV40short | 82 | 3407 |
| 30, 229 | bglob | 395 | 3408 |
| 31, 230 | bglobshort | 56 | 3409 |
| 34 | SV40polyA late (SL) | 181 | 3410 |
| 222 | T7 Tphi | 119 | 9336 |
| 223 | CaMV | 175 | 9337 |
| 224 | RDH1 | 171 | 9338 |
| 225 | Sv40 polyA late | 241 | 9339 |

Methods for plasmid nucleofection and assessing activity by FACS were conducted as described in Example 1.

### Neuronal Cell Culture:

All neuronal cell culture was performed using N2B327-based media. To induce neuronal differentiation, iPSCs were plated in neuronal plating media (N2B327 base media with 1 μg/mL doxycycline, 200 μM L-ascorbic acid, 1 μM dibutyryl cAMP sodium salt, 10 μM CultureOne, 100 ng/ml of BDNF, 100 ng/ml of GDNF). iNs (induced neurons) were dissociated, aliquoted, and frozen for long term storage after three days of differentiation (DIV3). DIV3 iNs were thawed and seeded on a 96-well plate at ˜30,000-50,000 cells per well. iNs were cultured for one week in plating media and thereafter, half-media changes were performed once every week using feeding media (N2B327 base media with 200 μM L-ascorbic acid, 1 μM dibutyryl cAMP sodium salt, 200 ng/ml of BDNF, 200 ng/ml of GDNF).

### AAV Transduction of iNs In Vitro:

24 hours prior to transduction, -30,000-50,000 iNs per well were seeded on Matrigel-coated 96-well plates. AAVs expressing the CasX:gRNA system, which included constructs encoding for poly(A) signal sequences listed in Table 12, were then diluted in neuronal plating media and added to cells. Cells were transduced at two MOIs (1E2 or 1E3 vg/cell). Seven days post-transduction, iNs were replenished using feeding media. Seven days post-transduction, cells were lifted using lysis buffer, 4-well replicates were pooled per experimental condition, and genomic DNA (gDNA) was harvested and prepared for editing analysis at the B2M locus using next generation sequencing (NGS).

### NGS Processing and Analysis:

Genomic DNA (gDNA) from harvested cells was extracted using the Zymo Quick-DNA™ Miniprep Plus kit following the manufacturer's instructions. Target amplicons were formed by amplifying regions of interest from 200 ng of extracted gDNA with a set of primers specific to the target locus, such as the human B2M gene. These gene-specific primers contained an additional sequence at the 5′ end to introduce an Illumina™ adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Cloning Poly(A) Signal Sequence Library for High-Throughput Screen:

To identify poly(A) signals that enable CasX to be expressed in an AAV genome in a high-throughput manner, a massively parallel reporter assay was conducted. Briefly, 10,000 poly(A) constructs (1,000 unique poly(A) signal sequences×10 barcodes per poly(A) signal sequence) were amplified, digested, and ligated into a restriction enzyme-digested AAV plasmid backbone harboring sequences coding for CasX protein 491 and gRNA scaffold variant 235 with spacer 7.37 (GGCCGAGAUGUCUCGCUCCG; SEQ ID NO: 4059) targeting the endogenous B2M (beta-2-microglobulin) locus. The 1000 unique poly(A) signal sequences, designated as Poly(A)_1 through Poly(A)_1001 (SEQ ID NOS: 2401-3401) are provided in Table 10. Cloned AAV plasmids were then transformed into electrocompetent bacterial cells (MegaX DH1B T1® Electrocomp™). Titer of poly(A) signal sequence library transformation was determined by counting E. coli colony-forming units (CFUs) from electroporated library yEGA-X Competent cells. After transformation and overnight growth in liquid cultures, the library was purified using the ZymoPURE™ Midiprep Kit. To determine adequate library coverage, barcoded amplicons were detected via PCR amplification followed by NGS on the Illumina™ MiSeq™. Raw fastq files were processed using cutadapt v3.5, mapped using bowtie2 v9.3.0, and barcodes were extracted using custom software. Barcoded counts were normalized by total read counts to calculate the representation of each library member.

TABLE 10
Poly(A) Signal Sequence Library

| | Poly(A) signal |
|---|---|
| Poly(A) | sequence |
| signal ID | (SEQ ID NO) |
| Poly(A)_1 | 2401 |
| Poly(A)_2 | 2402 |
| Poly(A)_3 | 2403 |
| Poly(A)_4 | 2404 |
| Poly(A)_5 | 2405 |
| Poly(A)_6 | 2406 |
| Poly(A)_7 | 2407 |
| Poly(A)_8 | 2408 |
| Poly(A)_9 | 2409 |
| Poly(A)_10 | 2410 |
| Poly(A)_11 | 2411 |
| Poly(A)_12 | 2412 |
| Poly(A)_13 | 2413 |
| Poly(A)_14 | 2414 |
| Poly(A)_15 | 2415 |
| Poly(A)_16 | 2416 |
| Poly(A)_17 | 2417 |
| Poly(A)_18 | 2418 |
| Poly(A)_19 | 2419 |
| Poly(A)_20 | 2420 |
| Poly(A)_21 | 2421 |
| Poly(A)_22 | 2422 |
| Poly(A)_23 | 2423 |
| Poly(A)_24 | 2424 |
| Poly(A)_25 | 2425 |
| Poly(A)_26 | 2426 |
| Poly(A)_27 | 2427 |
| Poly(A)_28 | 2428 |
| Poly(A)_29 | 2429 |
| Poly(A)_30 | 2430 |
| Poly(A)_31 | 2431 |
| Poly(A)_32 | 2432 |
| Poly(A)_33 | 2433 |
| Poly(A)_34 | 2434 |
| Poly(A)_35 | 2435 |
| Poly(A)_36 | 2436 |
| Poly(A)_37 | 2437 |
| Poly(A)_38 | 2438 |
| Poly(A)_39 | 2439 |
| Poly(A)_40 | 2440 |
| Poly(A)_41 | 2441 |
| Poly(A)_42 | 2442 |
| Poly(A)_43 | 2443 |
| Poly(A)_44 | 2444 |
| Poly(A)_45 | 2445 |
| Poly(A)_46 | 2446 |
| Poly(A)_47 | 2447 |
| Poly(A)_48 | 2448 |
| Poly(A)_49 | 2449 |
| Poly(A)_50 | 2450 |
| Poly(A)_51 | 2451 |
| Poly(A)_52 | 2452 |
| Poly(A)_53 | 2453 |
| Poly(A)_54 | 2454 |
| Poly(A)_55 | 2455 |
| Poly(A)_56 | 2456 |
| Poly(A)_57 | 2457 |
| Poly(A)_58 | 2458 |
| Poly(A)_59 | 2459 |
| Poly(A)_60 | 2460 |
| Poly(A)_61 | 2461 |
| Poly(A)_62 | 2462 |
| Poly(A)_63 | 2463 |
| Poly(A)_64 | 2464 |
| Poly(A)_65 | 2465 |
| Poly(A)_66 | 2466 |
| Poly(A)_67 | 2467 |
| Poly(A)_68 | 2468 |
| Poly(A)_69 | 2469 |
| Poly(A)_70 | 2470 |
| Poly(A)_71 | 2471 |
| Poly(A)_72 | 2472 |
| Poly(A)_73 | 2473 |
| Poly(A)_74 | 2474 |
| Poly(A)_75 | 2475 |
| Poly(A)_76 | 2476 |
| Poly(A)_77 | 2477 |
| Poly(A)_78 | 2478 |
| Poly(A)_79 | 2479 |
| Poly(A)_80 | 2480 |
| Poly(A)_81 | 2481 |
| Poly(A)_82 | 2482 |
| Poly(A)_83 | 2483 |
| Poly(A)_84 | 2484 |
| Poly(A)_85 | 2485 |
| Poly(A)_86 | 2486 |
| Poly(A)_87 | 2487 |
| Poly(A)_88 | 2488 |
| Poly(A)_89 | 2489 |
| Poly(A)_90 | 2490 |
| Poly(A)_91 | 2491 |
| Poly(A)_92 | 2492 |
| Poly(A)_93 | 2493 |
| Poly(A)_94 | 2494 |
| Poly(A)_95 | 2495 |
| Poly(A)_96 | 2496 |
| Poly(A)_97 | 2497 |
| Poly(A)_98 | 2498 |
| Poly(A)_99 | 2499 |
| Poly(A)_100 | 2500 |
| Poly(A)_101 | 2501 |
| Poly(A)_102 | 2502 |
| Poly(A)_103 | 2503 |
| Poly(A)_104 | 2504 |
| Poly(A)_105 | 2505 |
| Poly(A)_106 | 2506 |
| Poly(A)_107 | 2507 |
| Poly(A)_108 | 2508 |
| Poly(A)_109 | 2509 |
| Poly(A)_110 | 2510 |
| Poly(A)_111 | 2511 |
| Poly(A)_112 | 2512 |
| Poly(A)_113 | 2513 |
| Poly(A)_114 | 2514 |
| Poly(A)_115 | 2515 |
| Poly(A)_116 | 2516 |
| Poly(A)_117 | 2517 |
| Poly(A)_118 | 2518 |
| Poly(A)_119 | 2519 |
| Poly(A)_120 | 2520 |
| Poly(A)_121 | 2521 |
| Poly(A)_122 | 2522 |
| Poly(A)_123 | 2523 |
| Poly(A)_124 | 2524 |
| Poly(A)_125 | 2525 |
| Poly(A)_126 | 2526 |
| Poly(A)_127 | 2527 |
| Poly(A)_128 | 2528 |
| Poly(A)_129 | 2529 |
| Poly(A)_130 | 2530 |
| Poly(A)_131 | 2531 |
| Poly(A)_132 | 2532 |
| Poly(A)_133 | 2533 |
| Poly(A)_134 | 2534 |
| Poly(A)_135 | 2535 |
| Poly(A)_136 | 2536 |
| Poly(A)_137 | 2537 |
| Poly(A)_138 | 2538 |
| Poly(A)_139 | 2539 |
| Poly(A)_140 | 2540 |
| Poly(A)_141 | 2541 |
| Poly(A)_142 | 2542 |
| Poly(A)_143 | 2543 |
| Poly(A)_144 | 2544 |
| Poly(A)_145 | 2545 |
| Poly(A)_146 | 2546 |
| Poly(A)_147 | 2547 |
| Poly(A)_148 | 2548 |
| Poly(A)_149 | 2549 |
| Poly(A)_150 | 2550 |
| Poly(A)_151 | 2551 |
| Poly(A)_152 | 2552 |
| Poly(A)_153 | 2553 |
| Poly(A)_154 | 2554 |
| Poly(A)_155 | 2555 |
| Poly(A)_156 | 2556 |
| Poly(A)_157 | 2557 |
| Poly(A)_158 | 2558 |
| Poly(A)_159 | 2559 |
| Poly(A)_160 | 2560 |
| Poly(A)_161 | 2561 |
| Poly(A)_162 | 2562 |
| Poly(A)_163 | 2563 |
| Poly(A)_164 | 2564 |
| Poly(A)_165 | 2565 |
| Poly(A)_166 | 2566 |
| Poly(A)_167 | 2567 |
| Poly(A)_168 | 2568 |
| Poly(A)_169 | 2569 |
| Poly(A)_170 | 2570 |
| Poly(A)_171 | 2571 |
| Poly(A)_172 | 2572 |
| Poly(A)_173 | 2573 |
| Poly(A)_174 | 2574 |
| Poly(A)_175 | 2575 |
| Poly(A)_176 | 2576 |
| Poly(A)_177 | 2577 |
| Poly(A)_178 | 2578 |
| Poly(A)_179 | 2579 |
| Poly(A)_180 | 2580 |
| Poly(A)_181 | 2581 |
| Poly(A)_182 | 2582 |
| Poly(A)_183 | 2583 |
| Poly(A)_184 | 2584 |
| Poly(A)_185 | 2585 |
| Poly(A)_186 | 2586 |
| Poly(A)_187 | 2587 |
| Poly(A)_188 | 2588 |
| Poly(A)_189 | 2589 |
| Poly(A)_190 | 2590 |
| Poly(A)_191 | 2591 |
| Poly(A)_192 | 2592 |
| Poly(A)_193 | 2593 |
| Poly(A)_194 | 2594 |
| Poly(A)_195 | 2595 |
| Poly(A)_196 | 2596 |
| Poly(A)_197 | 2597 |
| Poly(A)_198 | 2598 |
| Poly(A)_199 | 2599 |
| Poly(A)_200 | 2600 |
| Poly(A)_201 | 2601 |
| Poly(A)_202 | 2602 |
| Poly(A)_203 | 2603 |
| Poly(A)_204 | 2604 |
| Poly(A)_205 | 2605 |
| Poly(A)_206 | 2606 |
| Poly(A)_207 | 2607 |
| Poly(A)_208 | 2608 |
| Poly(A)_209 | 2609 |
| Poly(A)_210 | 2610 |
| Poly(A)_211 | 2611 |
| Poly(A)_212 | 2612 |
| Poly(A)_213 | 2613 |
| Poly(A)_214 | 2614 |
| Poly(A)_215 | 2615 |
| Poly(A)_216 | 2616 |
| Poly(A)_217 | 2617 |
| Poly(A)_218 | 2618 |
| Poly(A)_219 | 2619 |
| Poly(A)_220 | 2620 |
| Poly(A)_221 | 2621 |
| Poly(A)_222 | 2622 |
| Poly(A)_223 | 2623 |
| Poly(A)_224 | 2624 |
| Poly(A)_225 | 2625 |
| Poly(A)_226 | 2626 |
| Poly(A)_227 | 2627 |
| Poly(A)_228 | 2628 |
| Poly(A)_229 | 2629 |
| Poly(A)_230 | 2630 |
| Poly(A)_231 | 2631 |
| Poly(A)_232 | 2632 |
| Poly(A)_233 | 2633 |
| Poly(A)_234 | 2634 |
| Poly(A)_235 | 2635 |
| Poly(A)_236 | 2636 |
| Poly(A)_237 | 2637 |
| Poly(A)_238 | 2638 |
| Poly(A)_239 | 2639 |
| Poly(A)_240 | 2640 |
| Poly(A)_241 | 2641 |
| Poly(A)_242 | 2642 |
| Poly(A)_243 | 2643 |
| Poly(A)_244 | 2644 |
| Poly(A)_245 | 2645 |
| Poly(A)_246 | 2646 |
| Poly(A)_247 | 2647 |
| Poly(A)_248 | 2648 |
| Poly(A)_249 | 2649 |
| Poly(A)_250 | 2650 |
| Poly(A)_251 | 2651 |
| Poly(A)_252 | 2652 |
| Poly(A)_253 | 2653 |
| Poly(A)_254 | 2654 |
| Poly(A)_255 | 2655 |
| Poly(A)_256 | 2656 |
| Poly(A)_257 | 2657 |
| Poly(A)_258 | 2658 |
| Poly(A)_259 | 2659 |
| Poly(A)_260 | 2660 |
| Poly(A)_261 | 2661 |
| Poly(A)_262 | 2662 |
| Poly(A)_263 | 2663 |
| Poly(A)_264 | 2664 |
| Poly(A)_265 | 2665 |
| Poly(A)_266 | 2666 |
| Poly(A)_267 | 2667 |
| Poly(A)_268 | 2668 |
| Poly(A)_269 | 2669 |
| Poly(A)_270 | 2670 |
| Poly(A)_271 | 2671 |
| Poly(A)_272 | 2672 |
| Poly(A)_273 | 2673 |
| Poly(A)_274 | 2674 |
| Poly(A)_275 | 2675 |
| Poly(A)_276 | 2676 |
| Poly(A)_277 | 2677 |
| Poly(A)_278 | 2678 |
| Poly(A)_279 | 2679 |
| Poly(A)_280 | 2680 |
| Poly(A)_281 | 2681 |
| Poly(A)_282 | 2682 |
| Poly(A)_283 | 2683 |
| Poly(A)_284 | 2684 |
| Poly(A)_285 | 2685 |
| Poly(A)_286 | 2686 |
| Poly(A)_287 | 2687 |
| Poly(A)_288 | 2688 |
| Poly(A)_289 | 2689 |
| Poly(A)_290 | 2690 |
| Poly(A)_291 | 2691 |
| Poly(A)_292 | 2692 |
| Poly(A)_293 | 2693 |
| Poly(A)_294 | 2694 |
| Poly(A)_295 | 2695 |
| Poly(A)_296 | 2696 |
| Poly(A)_297 | 2697 |
| Poly(A)_298 | 2698 |
| Poly(A)_299 | 2699 |
| Poly(A)_300 | 2700 |
| Poly(A)_301 | 2701 |
| Poly(A)_302 | 2702 |
| Poly(A)_303 | 2703 |
| Poly(A)_304 | 2704 |
| Poly(A)_305 | 2705 |
| Poly(A)_306 | 2706 |
| Poly(A)_307 | 2707 |
| Poly(A)_308 | 2708 |
| Poly(A)_309 | 2709 |
| Poly(A)_310 | 2710 |
| Poly(A)_311 | 2711 |
| Poly(A)_312 | 2712 |
| Poly(A)_313 | 2713 |
| Poly(A)_314 | 2714 |
| Poly(A)_315 | 2715 |
| Poly(A)_316 | 2716 |
| Poly(A)_317 | 2717 |
| Poly(A)_318 | 2718 |
| Poly(A)_319 | 2719 |
| Poly(A)_320 | 2720 |
| Poly(A)_321 | 2721 |
| Poly(A)_322 | 2722 |
| Poly(A)_323 | 2723 |
| Poly(A)_324 | 2724 |
| Poly(A)_325 | 2725 |
| Poly(A)_326 | 2726 |
| Poly(A)_327 | 2727 |
| Poly(A)_328 | 2728 |
| Poly(A)_329 | 2729 |
| Poly(A)_330 | 2730 |
| Poly(A)_331 | 2731 |
| Poly(A)_332 | 2732 |
| Poly(A)_333 | 2733 |
| Poly(A)_334 | 2734 |
| Poly(A)_335 | 2735 |
| Poly(A)_336 | 2736 |
| Poly(A)_337 | 2737 |
| Poly(A)_338 | 2738 |
| Poly(A)_339 | 2739 |
| Poly(A)_340 | 2740 |
| Poly(A)_341 | 2741 |
| Poly(A)_342 | 2742 |
| Poly(A)_343 | 2743 |
| Poly(A)_344 | 2744 |
| Poly(A)_345 | 2745 |
| Poly(A)_346 | 2746 |
| Poly(A)_347 | 2747 |
| Poly(A)_348 | 2748 |
| Poly(A)_349 | 2749 |
| Poly(A)_350 | 2750 |
| Poly(A)_351 | 2751 |
| Poly(A)_352 | 2752 |
| Poly(A)_353 | 2753 |
| Poly(A)_354 | 2754 |
| Poly(A)_355 | 2755 |
| Poly(A)_356 | 2756 |
| Poly(A)_357 | 2757 |
| Poly(A)_358 | 2758 |
| Poly(A)_359 | 2759 |
| Poly(A)_360 | 2760 |
| Poly(A)_361 | 2761 |
| Poly(A)_362 | 2762 |
| Poly(A)_363 | 2763 |
| Poly(A)_364 | 2764 |
| Poly(A)_365 | 2765 |
| Poly(A)_366 | 2766 |
| Poly(A)_367 | 2767 |
| Poly(A)_368 | 2768 |
| Poly(A)_369 | 2769 |
| Poly(A)_370 | 2770 |
| Poly(A)_371 | 2771 |
| Poly(A)_372 | 2772 |
| Poly(A)_373 | 2773 |
| Poly(A)_374 | 2774 |
| Poly(A)_375 | 2775 |
| Poly(A)_376 | 2776 |
| Poly(A)_377 | 2777 |
| Poly(A)_378 | 2778 |
| Poly(A)_379 | 2779 |
| Poly(A)_380 | 2780 |
| Poly(A)_381 | 2781 |
| Poly(A)_382 | 2782 |
| Poly(A)_383 | 2783 |
| Poly(A)_384 | 2784 |
| Poly(A)_385 | 2785 |
| Poly(A)_386 | 2786 |
| Poly(A)_387 | 2787 |
| Poly(A)_388 | 2788 |
| Poly(A)_389 | 2789 |
| Poly(A)_390 | 2790 |
| Poly(A)_391 | 2791 |
| Poly(A)_392 | 2792 |
| Poly(A)_393 | 2793 |
| Poly(A)_394 | 2794 |
| Poly(A)_395 | 2795 |
| Poly(A)_396 | 2796 |
| Poly(A)_397 | 2797 |
| Poly(A)_398 | 2798 |
| Poly(A)_399 | 2799 |
| Poly(A)_400 | 2800 |
| Poly(A)_401 | 2801 |
| Poly(A)_402 | 2802 |
| Poly(A)_403 | 2803 |
| Poly(A)_404 | 2804 |
| Poly(A)_405 | 2805 |
| Poly(A)_406 | 2806 |
| Poly(A)_407 | 2807 |
| Poly(A)_408 | 2808 |
| Poly(A)_409 | 2809 |
| Poly(A)_410 | 2810 |
| Poly(A)_411 | 2811 |
| Poly(A)_412 | 2812 |
| Poly(A)_413 | 2813 |
| Poly(A)_414 | 2814 |
| Poly(A)_415 | 2815 |
| Poly(A)_416 | 2816 |
| Poly(A)_417 | 2817 |
| Poly(A)_418 | 2818 |
| Poly(A)_419 | 2819 |
| Poly(A)_420 | 2820 |
| Poly(A)_421 | 2821 |
| Poly(A)_422 | 2822 |
| Poly(A)_423 | 2823 |
| Poly(A)_424 | 2824 |
| Poly(A)_425 | 2825 |
| Poly(A)_426 | 2826 |
| Poly(A)_427 | 2827 |
| Poly(A)_428 | 2828 |
| Poly(A)_429 | 2829 |
| Poly(A)_430 | 2830 |
| Poly(A)_431 | 2831 |
| Poly(A)_432 | 2832 |
| Poly(A)_433 | 2833 |
| Poly(A)_434 | 2834 |
| Poly(A)_435 | 2835 |
| Poly(A)_436 | 2836 |
| Poly(A)_437 | 2837 |
| Poly(A)_438 | 2838 |
| Poly(A)_439 | 2839 |
| Poly(A)_440 | 2840 |
| Poly(A)_441 | 2841 |
| Poly(A)_442 | 2842 |
| Poly(A)_443 | 2843 |
| Poly(A)_444 | 2844 |
| Poly(A)_445 | 2845 |
| Poly(A)_446 | 2846 |
| Poly(A)_447 | 2847 |
| Poly(A)_448 | 2848 |
| Poly(A)_449 | 2849 |
| Poly(A)_450 | 2850 |
| Poly(A)_451 | 2851 |
| Poly(A)_452 | 2852 |
| Poly(A)_453 | 2853 |
| Poly(A)_454 | 2854 |
| Poly(A)_455 | 2855 |
| Poly(A)_456 | 2856 |
| Poly(A)_457 | 2857 |
| Poly(A)_458 | 2858 |
| Poly(A)_459 | 2859 |
| Poly(A)_460 | 2860 |
| Poly(A)_461 | 2861 |
| Poly(A)_462 | 2862 |
| Poly(A)_463 | 2863 |
| Poly(A)_464 | 2864 |
| Poly(A)_465 | 2865 |
| Poly(A)_466 | 2866 |
| Poly(A)_467 | 2867 |
| Poly(A)_468 | 2868 |
| Poly(A)_469 | 2869 |
| Poly(A)_470 | 2870 |
| Poly(A)_471 | 2871 |
| Poly(A)_472 | 2872 |
| Poly(A)_473 | 2873 |
| Poly(A)_474 | 2874 |
| Poly(A)_475 | 2875 |
| Poly(A)_476 | 2876 |
| Poly(A)_477 | 2877 |
| Poly(A)_478 | 2878 |
| Poly(A)_479 | 2879 |
| Poly(A)_480 | 2880 |
| Poly(A)_481 | 2881 |
| Poly(A)_482 | 2882 |
| Poly(A)_483 | 2883 |
| Poly(A)_484 | 2884 |
| Poly(A)_485 | 2885 |
| Poly(A)_486 | 2886 |
| Poly(A)_487 | 2887 |
| Poly(A)_488 | 2888 |
| Poly(A)_489 | 2889 |
| Poly(A)_490 | 2890 |
| Poly(A)_491 | 2891 |
| Poly(A)_492 | 2892 |
| Poly(A)_493 | 2893 |
| Poly(A)_494 | 2894 |
| Poly(A)_495 | 2895 |
| Poly(A)_496 | 2896 |
| Poly(A)_497 | 2897 |
| Poly(A)_498 | 2898 |
| Poly(A)_499 | 2899 |
| Poly(A)_500 | 2900 |
| Poly(A)_501 | 2901 |
| Poly(A)_502 | 2902 |
| Poly(A)_503 | 2903 |
| Poly(A)_504 | 2904 |
| Poly(A)_505 | 2905 |
| Poly(A)_506 | 2906 |
| Poly(A)_507 | 2907 |
| Poly(A)_508 | 2908 |
| Poly(A)_509 | 2909 |
| Poly(A)_510 | 2910 |
| Poly(A)_511 | 2911 |
| Poly(A)_512 | 2912 |
| Poly(A)_513 | 2913 |
| Poly(A)_514 | 2914 |
| Poly(A)_515 | 2915 |
| Poly(A)_516 | 2916 |
| Poly(A)_517 | 2917 |
| Poly(A)_518 | 2918 |
| Poly(A)_519 | 2919 |
| Poly(A)_520 | 2920 |
| Poly(A)_521 | 2921 |
| Poly(A)_522 | 2922 |
| Poly(A)_523 | 2923 |
| Poly(A)_524 | 2924 |
| Poly(A)_525 | 2925 |
| Poly(A)_526 | 2926 |
| Poly(A)_527 | 2927 |
| Poly(A)_528 | 2928 |
| Poly(A)_529 | 2929 |
| Poly(A)_530 | 2930 |
| Poly(A)_531 | 2931 |
| Poly(A)_532 | 2932 |
| Poly(A)_533 | 2933 |
| Poly(A)_534 | 2934 |
| Poly(A)_535 | 2935 |
| Poly(A)_536 | 2936 |
| Poly(A)_537 | 2937 |
| Poly(A)_538 | 2938 |
| Poly(A)_539 | 2939 |
| Poly(A)_540 | 2940 |
| Poly(A)_541 | 2941 |
| Poly(A)_542 | 2942 |
| Poly(A)_543 | 2943 |
| Poly(A)_544 | 2944 |
| Poly(A)_545 | 2945 |
| Poly(A)_546 | 2946 |
| Poly(A)_547 | 2947 |
| Poly(A)_548 | 2948 |
| Poly(A)_549 | 2949 |
| Poly(A)_550 | 2950 |
| Poly(A)_551 | 2951 |
| Poly(A)_552 | 2952 |
| Poly(A)_553 | 2953 |
| Poly(A)_554 | 2954 |
| Poly(A)_555 | 2955 |
| Poly(A)_556 | 2956 |
| Poly(A)_557 | 2957 |
| Poly(A)_558 | 2958 |
| Poly(A)_559 | 2959 |
| Poly(A)_560 | 2960 |
| Poly(A)_561 | 2961 |
| Poly(A)_562 | 2962 |
| Poly(A)_563 | 2963 |
| Poly(A)_564 | 2964 |
| Poly(A)_565 | 2965 |
| Poly(A)_566 | 2966 |
| Poly(A)_567 | 2967 |
| Poly(A)_568 | 2968 |
| Poly(A)_569 | 2969 |
| Poly(A)_570 | 2970 |
| Poly(A)_571 | 2971 |
| Poly(A)_572 | 2972 |
| Poly(A)_573 | 2973 |
| Poly(A)_574 | 2974 |
| Poly(A)_575 | 2975 |
| Poly(A)_576 | 2976 |
| Poly(A)_577 | 2977 |
| Poly(A)_578 | 2978 |
| Poly(A)_579 | 2979 |
| Poly(A)_580 | 2980 |
| Poly(A)_581 | 2981 |
| Poly(A)_582 | 2982 |
| Poly(A)_583 | 2983 |
| Poly(A)_584 | 2984 |
| Poly(A)_585 | 2985 |
| Poly(A)_586 | 2986 |
| Poly(A)_587 | 2987 |
| Poly(A)_588 | 2988 |
| Poly(A)_589 | 2989 |
| Poly(A)_590 | 2990 |
| Poly(A)_591 | 2991 |
| Poly(A)_592 | 2992 |
| Poly(A)_593 | 2993 |
| Poly(A)_594 | 2994 |
| Poly(A)_595 | 2995 |
| Poly(A)_596 | 2996 |
| Poly(A)_597 | 2997 |
| Poly(A)_598 | 2998 |
| Poly(A)_599 | 2999 |
| Poly(A)_600 | 3000 |
| Poly(A)_601 | 3001 |
| Poly(A)_602 | 3002 |
| Poly(A)_603 | 3003 |
| Poly(A)_604 | 3004 |
| Poly(A)_605 | 3005 |
| Poly(A)_606 | 3006 |
| Poly(A)_607 | 3007 |
| Poly(A)_608 | 3008 |
| Poly(A)_609 | 3009 |
| Poly(A)_610 | 3010 |
| Poly(A)_611 | 3011 |
| Poly(A)_612 | 3012 |
| Poly(A)_613 | 3013 |
| Poly(A)_614 | 3014 |
| Poly(A)_615 | 3015 |
| Poly(A)_616 | 3016 |
| Poly(A)_617 | 3017 |
| Poly(A)_618 | 3018 |
| Poly(A)_619 | 3019 |
| Poly(A)_620 | 3020 |
| Poly(A)_621 | 3021 |
| Poly(A)_622 | 3022 |
| Poly(A)_623 | 3023 |
| Poly(A)_624 | 3024 |
| Poly(A)_625 | 3025 |
| Poly(A)_626 | 3026 |
| Poly(A)_627 | 3027 |
| Poly(A)_628 | 3028 |
| Poly(A)_629 | 3029 |
| Poly(A)_630 | 3030 |
| Poly(A)_631 | 3031 |
| Poly(A)_632 | 3032 |
| Poly(A)_633 | 3033 |
| Poly(A)_634 | 3034 |
| Poly(A)_635 | 3035 |
| Poly(A)_636 | 3036 |
| Poly(A)_637 | 3037 |
| Poly(A)_638 | 3038 |
| Poly(A)_639 | 3039 |
| Poly(A)_640 | 3040 |
| Poly(A)_641 | 3041 |
| Poly(A)_642 | 3042 |
| Poly(A)_643 | 3043 |
| Poly(A)_644 | 3044 |
| Poly(A)_645 | 3045 |
| Poly(A)_646 | 3046 |
| Poly(A)_647 | 3047 |
| Poly(A)_648 | 3048 |
| Poly(A)_649 | 3049 |
| Poly(A)_650 | 3050 |
| Poly(A)_651 | 3051 |
| Poly(A)_652 | 3052 |
| Poly(A)_653 | 3053 |
| Poly(A)_654 | 3054 |
| Poly(A)_655 | 3055 |
| Poly(A)_656 | 3056 |
| Poly(A)_657 | 3057 |
| Poly(A)_658 | 3058 |
| Poly(A)_659 | 3059 |
| Poly(A)_660 | 3060 |
| Poly(A)_661 | 3061 |
| Poly(A)_662 | 3062 |
| Poly(A)_663 | 3063 |
| Poly(A)_664 | 3064 |
| Poly(A)_665 | 3065 |
| Poly(A)_666 | 3066 |
| Poly(A)_667 | 3067 |
| Poly(A)_668 | 3068 |
| Poly(A)_669 | 3069 |
| Poly(A)_670 | 3070 |
| Poly(A)_671 | 3071 |
| Poly(A)_672 | 3072 |
| Poly(A)_673 | 3073 |
| Poly(A)_674 | 3074 |
| Poly(A)_675 | 3075 |
| Poly(A)_676 | 3076 |
| Poly(A)_677 | 3077 |
| Poly(A)_678 | 3078 |
| Poly(A)_679 | 3079 |
| Poly(A)_680 | 3080 |
| Poly(A)_681 | 3081 |
| Poly(A)_682 | 3082 |
| Poly(A)_683 | 3083 |
| Poly(A)_684 | 3084 |
| Poly(A)_685 | 3085 |
| Poly(A)_686 | 3086 |
| Poly(A)_687 | 3087 |
| Poly(A)_688 | 3088 |
| Poly(A)_689 | 3089 |
| Poly(A)_690 | 3090 |
| Poly(A)_691 | 3091 |
| Poly(A)_692 | 3092 |
| Poly(A)_693 | 3093 |
| Poly(A)_694 | 3094 |
| Poly(A)_695 | 3095 |
| Poly(A)_696 | 3096 |
| Poly(A)_697 | 3097 |
| Poly(A)_698 | 3098 |
| Poly(A)_699 | 3099 |
| Poly(A)_700 | 3100 |
| Poly(A)_701 | 3101 |
| Poly(A)_702 | 3102 |
| Poly(A)_703 | 3103 |
| Poly(A)_704 | 3104 |
| Poly(A)_705 | 3105 |
| Poly(A)_706 | 3106 |
| Poly(A)_707 | 3107 |
| Poly(A)_708 | 3108 |
| Poly(A)_709 | 3109 |
| Poly(A)_710 | 3110 |
| Poly(A)_711 | 3111 |
| Poly(A)_712 | 3112 |
| Poly(A)_713 | 3113 |
| Poly(A)_714 | 3114 |
| Poly(A)_715 | 3115 |
| Poly(A)_716 | 3116 |
| Poly(A)_717 | 3117 |
| Poly(A)_718 | 3118 |
| Poly(A)_719 | 3119 |
| Poly(A)_720 | 3120 |
| Poly(A)_721 | 3121 |
| Poly(A)_722 | 3122 |
| Poly(A)_723 | 3123 |
| Poly(A)_724 | 3124 |
| Poly(A)_725 | 3125 |
| Poly(A)_726 | 3126 |
| Poly(A)_727 | 3127 |
| Poly(A)_728 | 3128 |
| Poly(A)_729 | 3129 |
| Poly(A)_730 | 3130 |
| Poly(A)_731 | 3131 |
| Poly(A)_732 | 3132 |
| Poly(A)_733 | 3133 |
| Poly(A)_734 | 3134 |
| Poly(A)_735 | 3135 |
| Poly(A)_736 | 3136 |
| Poly(A)_737 | 3137 |
| Poly(A)_738 | 3138 |
| Poly(A)_739 | 3139 |
| Poly(A)_740 | 3140 |
| Poly(A)_741 | 3141 |
| Poly(A)_742 | 3142 |
| Poly(A)_743 | 3143 |
| Poly(A)_744 | 3144 |
| Poly(A)_745 | 3145 |
| Poly(A)_746 | 3146 |
| Poly(A)_747 | 3147 |
| Poly(A)_748 | 3148 |
| Poly(A)_749 | 3149 |
| Poly(A)_750 | 3150 |
| Poly(A)_751 | 3151 |
| Poly(A)_752 | 3152 |
| Poly(A)_753 | 3153 |
| Poly(A)_754 | 3154 |
| Poly(A)_755 | 3155 |
| Poly(A)_756 | 3156 |
| Poly(A)_757 | 3157 |
| Poly(A)_758 | 3158 |
| Poly(A)_759 | 3159 |
| Poly(A)_760 | 3160 |
| Poly(A)_761 | 3161 |
| Poly(A)_762 | 3162 |
| Poly(A)_763 | 3163 |
| Poly(A)_764 | 3164 |
| Poly(A)_765 | 3165 |
| Poly(A)_766 | 3166 |
| Poly(A)_767 | 3167 |
| Poly(A)_768 | 3168 |
| Poly(A)_769 | 3169 |
| Poly(A)_770 | 3170 |
| Poly(A)_771 | 3171 |
| Poly(A)_772 | 3172 |
| Poly(A)_773 | 3173 |
| Poly(A)_774 | 3174 |
| Poly(A)_775 | 3175 |
| Poly(A)_776 | 3176 |
| Poly(A)_777 | 3177 |
| Poly(A)_778 | 3178 |
| Poly(A)_779 | 3179 |
| Poly(A)_780 | 3180 |
| Poly(A)_781 | 3181 |
| Poly(A)_782 | 3182 |
| Poly(A)_783 | 3183 |
| Poly(A)_784 | 3184 |
| Poly(A)_785 | 3185 |
| Poly(A)_786 | 3186 |
| Poly(A)_787 | 3187 |
| Poly(A)_788 | 3188 |
| Poly(A)_789 | 3189 |
| Poly(A)_790 | 3190 |
| Poly(A)_791 | 3191 |
| Poly(A)_792 | 3192 |
| Poly(A)_793 | 3193 |
| Poly(A)_794 | 3194 |
| Poly(A)_795 | 3195 |
| Poly(A)_796 | 3196 |
| Poly(A)_797 | 3197 |
| Poly(A)_798 | 3198 |
| Poly(A)_799 | 3199 |
| Poly(A)_800 | 3200 |
| Poly(A)_801 | 3201 |
| Poly(A)_802 | 3202 |
| Poly(A)_803 | 3203 |
| Poly(A)_804 | 3204 |
| Poly(A)_805 | 3205 |
| Poly(A)_806 | 3206 |
| Poly(A)_807 | 3207 |
| Poly(A)_808 | 3208 |
| Poly(A)_809 | 3209 |
| Poly(A)_810 | 3210 |
| Poly(A)_811 | 3211 |
| Poly(A)_812 | 3212 |
| Poly(A)_813 | 3213 |
| Poly(A)_814 | 3214 |
| Poly(A)_815 | 3215 |
| Poly(A)_816 | 3216 |
| Poly(A)_817 | 3217 |
| Poly(A)_818 | 3218 |
| Poly(A)_819 | 3219 |
| Poly(A)_820 | 3220 |
| Poly(A)_821 | 3221 |
| Poly(A)_822 | 3222 |
| Poly(A)_823 | 3223 |
| Poly(A)_824 | 3224 |
| Poly(A)_825 | 3225 |
| Poly(A)_826 | 3226 |
| Poly(A)_827 | 3227 |
| Poly(A)_828 | 3228 |
| Poly(A)_829 | 3229 |
| Poly(A)_830 | 3230 |
| Poly(A)_831 | 3231 |
| Poly(A)_832 | 3232 |
| Poly(A)_833 | 3233 |
| Poly(A)_834 | 3234 |
| Poly(A)_835 | 3235 |
| Poly(A)_836 | 3236 |
| Poly(A)_837 | 3237 |
| Poly(A)_838 | 3238 |
| Poly(A)_839 | 3239 |
| Poly(A)_840 | 3240 |
| Poly(A)_841 | 3241 |
| Poly(A)_842 | 3242 |
| Poly(A)_843 | 3243 |
| Poly(A)_844 | 3244 |
| Poly(A)_845 | 3245 |
| Poly(A)_846 | 3246 |
| Poly(A)_847 | 3247 |
| Poly(A)_848 | 3248 |
| Poly(A)_849 | 3249 |
| Poly(A)_850 | 3250 |
| Poly(A)_851 | 3251 |
| Poly(A)_852 | 3252 |
| Poly(A)_853 | 3253 |
| Poly(A)_854 | 3254 |
| Poly(A)_855 | 3255 |
| Poly(A)_856 | 3256 |
| Poly(A)_857 | 3257 |
| Poly(A)_858 | 3258 |
| Poly(A)_859 | 3259 |
| Poly(A)_860 | 3260 |
| Poly(A)_861 | 3261 |
| Poly(A)_862 | 3262 |
| Poly(A)_863 | 3263 |
| Poly(A)_864 | 3264 |
| Poly(A)_865 | 3265 |
| Poly(A)_866 | 3266 |
| Poly(A)_867 | 3267 |
| Poly(A)_868 | 3268 |
| Poly(A)_869 | 3269 |
| Poly(A)_870 | 3270 |
| Poly(A)_871 | 3271 |
| Poly(A)_872 | 3272 |
| Poly(A)_873 | 3273 |
| Poly(A)_874 | 3274 |
| Poly(A)_875 | 3275 |
| Poly(A)_876 | 3276 |
| Poly(A)_877 | 3277 |
| Poly(A)_878 | 3278 |
| Poly(A)_879 | 3279 |
| Poly(A)_880 | 3280 |
| Poly(A)_881 | 3281 |
| Poly(A)_882 | 3282 |
| Poly(A)_883 | 3283 |
| Poly(A)_884 | 3284 |
| Poly(A)_885 | 3285 |
| Poly(A)_886 | 3286 |
| Poly(A)_887 | 3287 |
| Poly(A)_888 | 3288 |
| Poly(A)_889 | 3289 |
| Poly(A)_890 | 3290 |
| Poly(A)_891 | 3291 |
| Poly(A)_892 | 3292 |
| Poly(A)_893 | 3293 |
| Poly(A)_894 | 3294 |
| Poly(A)_895 | 3295 |
| Poly(A)_896 | 3296 |
| Poly(A)_897 | 3297 |
| Poly(A)_898 | 3298 |
| Poly(A)_899 | 3299 |
| Poly(A)_900 | 3300 |
| Poly(A)_901 | 3301 |
| Poly(A)_902 | 3302 |
| Poly(A)_903 | 3303 |
| Poly(A)_904 | 3304 |
| Poly(A)_905 | 3305 |
| Poly(A)_906 | 3306 |
| Poly(A)_907 | 3307 |
| Poly(A)_908 | 3308 |
| Poly(A)_909 | 3309 |
| Poly(A)_910 | 3310 |
| Poly(A)_911 | 3311 |
| Poly(A)_912 | 3312 |
| Poly(A)_913 | 3313 |
| Poly(A)_914 | 3314 |
| Poly(A)_915 | 3315 |
| Poly(A)_916 | 3316 |
| Poly(A)_917 | 3317 |
| Poly(A)_918 | 3318 |
| Poly(A)_919 | 3319 |
| Poly(A)_920 | 3320 |
| Poly(A)_921 | 3321 |
| Poly(A)_922 | 3322 |
| Poly(A)_923 | 3323 |
| Poly(A)_924 | 3324 |
| Poly(A)_925 | 3325 |
| Poly(A)_926 | 3326 |
| Poly(A)_927 | 3327 |
| Poly(A)_928 | 3328 |
| Poly(A)_929 | 3329 |
| Poly(A)_930 | 3330 |
| Poly(A)_931 | 3331 |
| Poly(A)_932 | 3332 |
| Poly(A)_933 | 3333 |
| Poly(A)_934 | 3334 |
| Poly(A)_935 | 3335 |
| Poly(A)_936 | 3336 |
| Poly(A)_937 | 3337 |
| Poly(A)_938 | 3338 |
| Poly(A)_939 | 3339 |
| Poly(A)_940 | 3340 |
| Poly(A)_941 | 3341 |
| Poly(A)_942 | 3342 |
| Poly(A)_943 | 3343 |
| Poly(A)_944 | 3344 |
| Poly(A)_945 | 3345 |
| Poly(A)_946 | 3346 |
| Poly(A)_947 | 3347 |
| Poly(A)_948 | 3348 |
| Poly(A)_949 | 3349 |
| Poly(A)_950 | 3350 |
| Poly(A)_951 | 3351 |
| Poly(A)_952 | 3352 |
| Poly(A)_953 | 3353 |
| Poly(A)_954 | 3354 |
| Poly(A)_955 | 3355 |
| Poly(A)_956 | 3356 |
| Poly(A)_957 | 3357 |
| Poly(A)_958 | 3358 |
| Poly(A)_959 | 3359 |
| Poly(A)_960 | 3360 |
| Poly(A)_961 | 3361 |
| Poly(A)_962 | 3362 |
| Poly(A)_963 | 3363 |
| Poly(A)_964 | 3364 |
| Poly(A)_965 | 3365 |
| Poly(A)_966 | 3366 |
| Poly(A)_967 | 3367 |
| Poly(A)_968 | 3368 |
| Poly(A)_969 | 3369 |
| Poly(A)_970 | 3370 |
| Poly(A)_971 | 3371 |
| Poly(A)_972 | 3372 |
| Poly(A)_973 | 3373 |
| Poly(A)_974 | 3374 |
| Poly(A)_975 | 3375 |
| Poly(A)_976 | 3376 |
| Poly(A)_977 | 3377 |
| Poly(A)_978 | 3378 |
| Poly(A)_979 | 3379 |
| Poly(A)_980 | 3380 |
| Poly(A)_981 | 3381 |
| Poly(A)_982 | 3382 |
| Poly(A)_983 | 3383 |
| Poly(A)_984 | 3384 |
| Poly(A)_985 | 3385 |
| Poly(A)_986 | 3386 |
| Poly(A)_987 | 3387 |
| Poly(A)_988 | 3388 |
| Poly(A)_989 | 3389 |
| Poly(A)_990 | 3390 |
| Poly(A)_991 | 3391 |
| Poly(A)_992 | 3392 |
| Poly(A)_993 | 3393 |
| Poly(A)_994 | 3394 |
| Poly(A)_995 | 3395 |
| Poly(A)_996 | 3396 |
| Poly(A)_997 | 3397 |
| Poly(A)_998 | 3398 |
| Poly(A)_999 | 3399 |
| Poly(A)_1000 | 3400 |
| Poly(A)_1001 | 3401 |

### AAV Vector Production:

AAV vectors were produced according to standard methods, which are described in Example 1.

To determine the viral genome (vg) titer, 1 μL from crude lysate viruses was digested with DNase and Proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. Ten-fold serial dilutions of an AAV ITR plasmid was used as reference standards to calculate the titer (vg/mL) of viral samples.

After production, AAVs from the pooled library were lysed to release AAV virion DNA, which was then purified according to standard methods. Barcoded amplicons were PCR-amplified from the viral DNA input, sequenced, and processed as described earlier to determine the coverage of the AAV pool. Barcode counts were normalized by total read counts to calculate an RPM value.

### AAV Transduction and Method for RNA Transcript Analysis:

10,000 HEK293T cells were seeded per well in PLF-coated 24-well plates 48 hours before AAV transduction. At the time of transduction, HEK293 Ts were transduced with the pooled library of AAVs containing the library of poly(A) signal sequences. All viral infection conditions were performed in triplicate, with normalized number of vg among experimental vectors, at an MOI of 1E5 and 1E4 vg/cell. Two days post-transduction, total RNA was isolated and converted into cDNA by reverse transcription. Barcoded amplicons were PCR-amplified from the resulting cDNA, sequenced, and processed as described earlier. Barcode counts were normalized by total read counts to calculate an RPM value. To calculate the RNA abundance ratio for each poly(A) signal sequence from the library, normalized barcode counts from cDNA amplicons were divided by normalized barcode counts from viral DNA input. Poly(A) signal sequences with a high RNA abundance ratio, i.e., with the highest accumulation in HEK293 Ts, were identified as the poly(A) signal sequences of interest for further CasX editing assessments in vitro or in vivo.

### Results

The results portrayed in FIGS. 22 and 23 demonstrate that AAV constructs with several alternative poly(A) signals, in combination with CasX variant 491, scaffold variant 174 and spacer 12.7, when delivered by nucleofection of AAV transgene plasmid, were able to edit the target stop cassette in mNPCs at doses of 250 ng and 125 ng. Construct AAV3 (bGH poly(A) signal sequence) showed the highest potency out of the three constructs tested in this experiment, editing the target locus at 60% efficiency (250 ng dose). Constructs 28 (SV40) and 29 (SV40 short), which have poly(A) signal sequences that are 59% and 39% of the size of the poly(A) signal sequence of construct 3, respectively (see Table 11), edited at 21% and 24% respectively (250 ng dose).

TABLE 11
AAV constructs with poly(A) signal sequence variants.

| | Poly(A) Signal | |
|---|---|---|
| Construct | Sequence | AAV Transgene |
| ID | Length (bp) | Length (bp) |
| 3 | 208 | 4550 |
| 25 | 477 | 4795 |
| 26 | 49 | 4367 |
| 27 | 49 | 4367 |
| 28 | 122 | 4440 |
| 29 | 82 | 4400 |
| 30 | 395 | 4713 |
| 31 | 56 | 4374 |
| 34 | 181 | 4565 |
| 37 | 208 | 4619 |

The results portrayed in FIG. 23 demonstrate that the two different poly(A) signals, combined with CasX protein 491, scaffold variant 174 and spacer 12.7, when delivered as an AAV vector, were able to edit the target stop cassette in mNPCs. AAV.34 and AAV.37 were generated with transgene constructs 34 (with a poly(A) signal of 186 nucleotides and a total transgene length of 4565 nucleotides) and 37 (with a poly(A) signal of 208 nucleotides and a total transgene length of 4619 nucleotides), respectively. Each vector displayed dose-dependent editing at the target locus, and AAV.34, which contains a shorter poly(A) signal, had approximately 75% of the editing potency of AAV.37 for both doses.

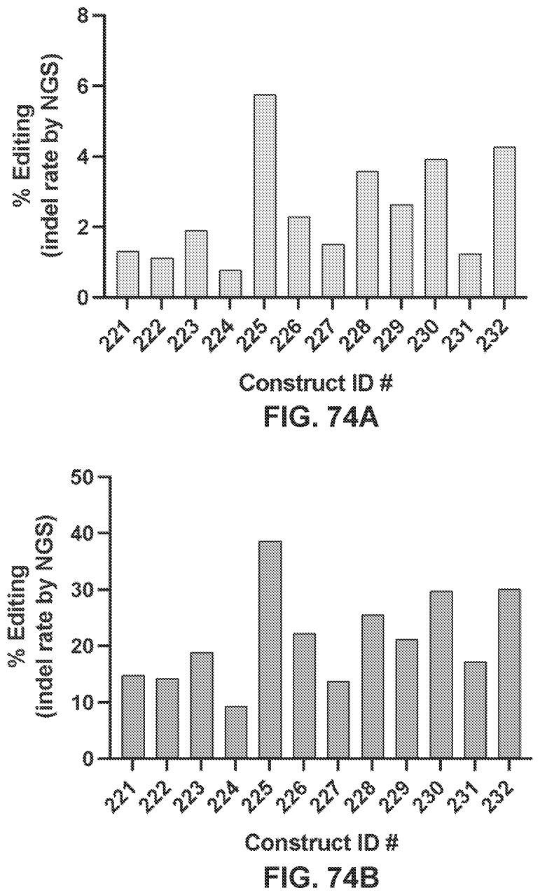

The results portrayed in FIGS. 74A-74B demonstrate that use of AAV constructs containing the SV40 poly(A) late poly(A) signal (construct ID 225) resulted in improved editing compared to that when using constructs with other poly(A) signals. Furthermore, multiple constructs containing poly(A) signals less than 70 bp contained high activity. Each vector displayed dose-dependent editing at the target locus.

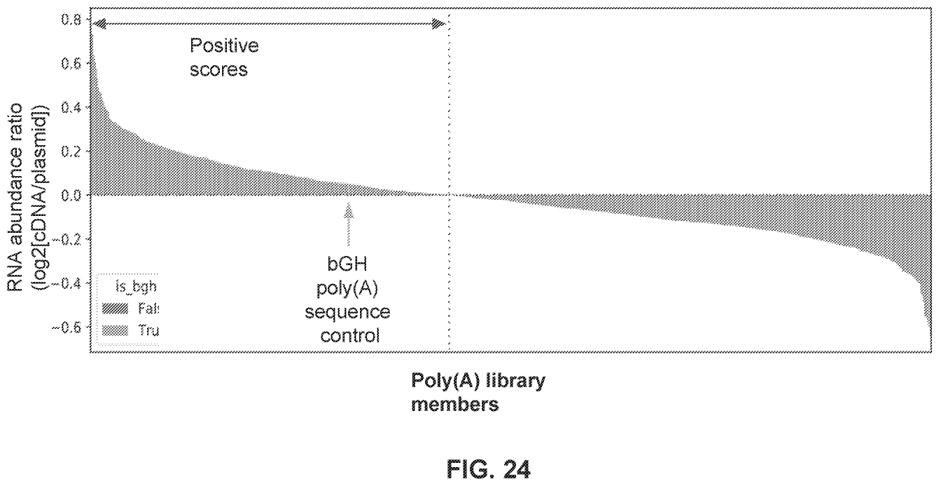

Experiments were performed in HEK293T cells to screen for poly(A) signal sequences for incorporation into future AAV construct designs that would improve CasX expression. As described above, poly(A) signal sequences with a high RNA abundance ratio would be identified as the poly(A) signal sequences of interest for further testing. The RNA abundance ratio was calculated across ten technical replicates by summing the counts across technical replicates and plotted for each unique poly(A) signal sequence from the library for each biological replicate (FIG. 24). Approximately 42% of poly(A) signal sequences screened demonstrated a positive RNA abundance ratio in any of the three biological replicates assessed, indicating that use of these poly(A) signal sequences resulted in higher CasX expression. Here, the bGH poly(A) signal sequence served as a positive control and is annotated in FIG. 24. The mean RNA abundance ratio was also calculated and plotted against the sequence length for each poly(A) signal candidate (data not shown). It was determined that approximately 71% of the poly(A) signal sequences with a positive RNA abundance ratio in any of the three biological replicates also have a sequence length shorter than the sequence of the bGH control (109 bp) from start of the sequence to polyadenylation site. A list of poly(A) signal sequences with a positive mean RNA abundance ratio across all three biological replicates and with a sequence length shorter than bGH across all three biological replicates is presented in Table 12. These identified poly(A) signal sequences, as well as sequences listed in Table 13, are incorporated in future AAV construct designs for further assessment in vitro or in vivo. The findings here support use of the unique poly(A) signal sequences in designing AAV vectors that would provide additional flexibility for increased AAV transgene cargo capacity while potentially enhancing CasX expression and editing efficiency.

Overall, the results demonstrate that the expression of small CRISPR proteins, such as CasX, can be modulated by poly(A) signals of varying lengths. Longer poly(A) signal sequences can be utilized in the AAV constructs for enhanced CasX activity, while shorter poly(A) signal sequences can be utilized in the AAV constructs to make more sequence space available for the inclusion of additional accessory elements within the AAV transgene.

TABLE 12
List of poly(A) signals identified from a high-throughput screen that
demonstrated a positive mean RNA abundance ratio observed in three biological
replicates and harbor a sequence length shorter than the bGH control (109 bp).

| | Poly(A) | | | Poly(A) | |
|---|---|---|---|---|---|
| Name | signal ID | SEQ ID NO | Name | signal ID | SEQ ID NO |
| SPATA24 | Poly(A)_812 | 3212 | CCDC180 | Poly(A)_946 | 3346 |
| C14orf28 | Poly(A)_269 | 2668 | KCNJ13 | Poly(A)_645 | 3045 |
| TSKS | Poly(A)_554 | 2954 | SCO2 | Poly(A)_707 | 3107 |
| NUP153 | Poly(A)_833 | 3233 | TUT1 | Poly(A)_145 | 2545 |
| RPS12 | Poly(A)_862 | 3262 | PTH2 | Poly(A)_543 | 2943 |
| IL17RC | Poly(A)_709 | 3109 | THOCI | Poly(A)_442 | 2842 |
| CALM2 | Poly(A)_594 | 2994 | ACKR1 | Poly(A)_76 | 2476 |
| KLK13 | Poly(A)_562 | 2962 | RPL38 | Poly(A)_429 | 2829 |
| STPG4 | Poly(A)_593 | 2993 | SAMD8 | Poly(A)_102 | 2502 |
| RNF181 | Poly(A)_612 | 3012 | RPS8 | Poly(A)_33 | 2433 |
| SPAG4 | Poly(A)_663 | 3063 | SCNM1 | Poly(A)_55 | 2455 |
| RHOT2 | Poly(A)_306 | 2706 | HEXDC | Poly(A)_441 | 2841 |
| GLG1 | Poly(A)_359 | 2759 | EXOSC4 | Poly(A)_931 | 3331 |
| ACTA2 | Poly(A)_105 | 2505 | IDH3B | Poly(A)_652 | 3052 |
| SMPD2 | Poly(A)_859 | 3259 | UBE2D2 | Poly(A)_813 | 3213 |
| MRPL52 | Poly(A)_254 | 2654 | CBR3 | Poly(A)_678 | 3078 |
| ETV2 | Poly(A)_499 | 2899 | GPR19 | Poly(A)_195 | 2595 |
| TMEM102 | Poly(A)_383 | 2783 | ALKBH7 | Poly(A)_462 | 2862 |
| CLCNKB | Poly(A)_13 | 2413 | UBE2D3 | Poly(A)_782 | 3182 |
| AAAS | Poly(A)_203 | 2603 | SUPV3L1 | Poly(A)_99 | 2499 |
| SPATA7 | Poly(A)_273 | 2673 | SENP3 | Poly(A)_384 | 2784 |
| FKBP11 | Poly(A)_200 | 2600 | RNF166 | Poly(A)_366 | 2766 |
| NOP16 | Poly(A)_821 | 3221 | EEF1D | Poly(A)_927 | 3327 |
| CDC34 | Poly(A)_450 | 2850 | TEKT1 | Poly(A)_380 | 2780 |
| NLRX1 | Poly(A)_188 | 2588 | PSTK | Poly(A)_115 | 2515 |
| PRPF8 | Poly(A)_370 | 2770 | RETREG1 | Poly(A)_792 | 3192 |
| EIF3K | Poly(A)_508 | 2908 | RPL7 | Poly(A)_910 | 3310 |
| KRTCAP2 | Poly(A)_71 | 2471 | SLIT2 | Poly(A)_775 | 3175 |
| RPS15 | Poly(A)_455 | 2855 | CALHM6 | Poly(A)_861 | 3261 |
| ELOC | Poly(A)_911 | 3311 | Clorf53 | Poly(A)_87 | 2487 |
| TFPT | Poly(A)_568 | 2968 | AURKAIP1 | Poly(A)_3 | 2403 |
| CEP350 | Poly(A)_86 | 2486 | EEF1D | Poly(A)_928 | 3328 |
| RAB33A | Poly(A)_993 | 3393 | CRYGD | Poly(A)_636 | 3036 |
| WDR36 | Poly(A)_808 | 3208 | MSRB3 | Poly(A)_215 | 2615 |
| ARMC12 | Poly(A)_846 | 3246 | ABHD1 | Poly(A)_584 | 2984 |
| NNMT | Poly(A)_184 | 2584 | HCCS | Poly(A)_972 | 3372 |
| NAA50 | Poly(A)_744 | 3144 | POLD1 | Poly(A)_558 | 2958 |
| RPL14 | Poly(A)_716 | 3116 | ZBTB48 | Poly(A)_7 | 2407 |
| RPL18 | Poly(A)_537 | 2937 | C17orf98 | Poly(A)_405 | 2805 |
| TMEM205 | Poly(A)_474 | 2874 | ATRIP | Poly(A)_721 | 3121 |
| MRPL52 | Poly(A)_253 | 2721 | TRO | Poly(A)_983 | 3383 |
| CORO7- | Poly(A)_321 | 2470 | U2AF1L4 | Poly(A)_502 | 2902 |
| PAM16 | | | | | |
| DPM3 | Poly(A)_70 | 2970 | PRIM1 | Poly(A)_211 | 2611 |
| RPS9 | Poly(A)_570 | | S100A2 | Poly(A)_65 | 2464 |
| SYCEIL | Poly(A)_362 | 2762 | PRKAA1 | Poly(A)_794 | 3194 |
| AKAP8L | Poly(A)_479 | 2879 | CPVL | Poly(A)_870 | 3270 |
| DHX30 | Poly(A)_719 | 3119 | PIP | Poly(A)_896 | 3296 |
| RABGGTA | Poly(A)_265 | 2665 | MAPKAPK5 | Poly(A)_227 | 2627 |
| RNF181 | Poly(A)_611 | 3011 | IL13RA2 | Poly(A)_988 | 3388 |
| PPP1R35 | Poly(A)_881 | 3281 | C2orf70 | Poly(A)_582 | 2982 |
| RRP9 | Poly(A)_735 | 3135 | TMEM219 | Poly(A)_331 | 2731 |
| VPS29 | Poly(A)_225 | 2625 | EXOSC8 | Poly(A)_238 | 2638 |
| NUP85 | Poly(A)_431 | 2831 | CUTA | Poly(A)_843 | 3243 |
| EXOC3L1 | Poly(A)_347 | 2747 | PLCB2 | Poly(A)_282 | 2682 |
| TAF10 | Poly(A)_126 | 2526 | PCGF1 | Poly(A)_608 | 3008 |
| PPP1R13L | Poly(A)_531 | 2931 | PFDN5 | Poly(A)_201 | 2601 |
| POLB | Poly(A)_907 | 3307 | IFT27 | Poly(A)_697 | 3097 |
| INTS8 | Poly(A)_916 | 3316 | VMA21 | Poly(A)_994 | 3394 |
| PLA2G1B | Poly(A)_229 | 2629 | PSMB1 | Poly(A)_864 | 3264 |
| ZFAND5 | Poly(A)_945 | 3345 | EEF1D | Poly(A)_929 | 3329 |
| RPL30 | Poly(A)_918 | 3318 | ARMC3 | Poly(A)_98 | 2498 |
| SGF29 | Poly(A)_329 | 2729 | AP1M1 | Poly(A)_480 | 2880 |
| NUDT14 | Poly(A)_279 | 2679 | BOLA3 | Poly(A)_604 | 3004 |
| IFT122 | Poly(A)_751 | 3151 | TMEM258 | Poly(A)_143 | 2543 |
| CUTA | Poly(A)_842 | 3242 | TCTEX1D2 | Poly(A)_764 | 3164 |
| FBL | Poly(A)_516 | 2916 | ALAS1 | Poly(A)_737 | 3137 |
| ZCRB1 | Poly(A)_197 | 2597 | AKAP14 | Poly(A)_991 | 3391 |
| C2orf70 | Poly(A)_583 | 2983 | TEX10 | Poly(A)_947 | 3347 |
| CCDC33 | Poly(A)_291 | 2691 | CTNNBL1 | Poly(A)_665 | 3065 |
| MPHOSPH10 | Poly(A)_603 | 3003 | UBL5 | Poly(A)_472 | 2872 |
| BAG6 | Poly(A)_836 | 3236 | FGF21 | Poly(A)_540 | 2940 |
| C11orf80 | Poly(A)_167 | 2567 | PPP2R3C | Poly(A)_268 | 2668 |
| PPIL6 | Poly(A)_858 | 3258 | SEPT11 | Poly(A)_778 | 3178 |
| SNRNP27 | Poly(A)_600 | 3000 | | | |
| NUP93 | Poly(A)_339 | 2739 | | | |

TABLE 13
List of additional poly(A) signal sequences for incorporation
into future AAV construct designs and further testing.

| | Poly(A) | SEQ |
|---|---|---|
| Name | signal ID | ID NO |
| RPL10 | Poly(A)_1000 | 3400 |
| RPL11 | Poly(A)_16 | 2416 |
| RPL12 | Poly(A)_952 | 3352 |
| RPL13A | Poly(A)_546 | 2946 |
| RPL22 | Poly(A)_6 | 2406 |
| RPL22L1 | Poly(A)_757 | 3157 |
| RPL26 | Poly(A)_390 | 2790 |
| RPL26 | Poly(A)_391 | 2791 |
| RPL26 | Poly(A)_392 | 2792 |
| RPL27A | Poly(A)_127 | 2527 |
| RPL3 | Poly(A)_700 | 3100 |
| RPL30 | Poly(A)_919 | 3319 |
| RPL32 | Poly(A)_713 | 3113 |
| RPL35A | Poly(A)_765 | 3165 |
| RPL35A | Poly(A)_766 | 3166 |
| RPL5 | Poly(A)_43 | 2442 |
| RPL8 | Poly(A)_935 | 3335 |
| RPS11 | Poly(A)_547 | 2947 |
| RPS15A | Poly(A)_326 | 2726 |
| RPS16 | Poly(A)_513 | 2913 |
| RPS16 | Poly(A)_514 | 2914 |
| RPS19 | Poly(A)_521 | 2921 |
| RPS3A | Poly(A)_786 | 3186 |
| RPS5 | Poly(A)_574 | 2974 |
| RPS7 | Poly(A)_578 | 2978 |

### Example 7: Small CRISPR Protein Potency is Modulated by the Position of Regulatory Elements in the AAV Vector

Orientation (forward or reverse) and position (upstream or downstream of CRISPR gene) of regulatory elements such as the gRNA promoter and guide scaffold complex can modulate the underlying expression of the small CRISPR protein and the overall editing efficiency of CRISPR systems in AAV vectors. Experiments were performed to assess the best orientation and position of regulatory elements within the AAV genome to enhance the potency of small CRISPR proteins and guide RNAs.

### Materials and Methods

AAV vector production and QC, nucleofection, AAV viral production and editing level assessment in mNPTC-tdT cells by FACS were conducted as described in Example 1.

### Results

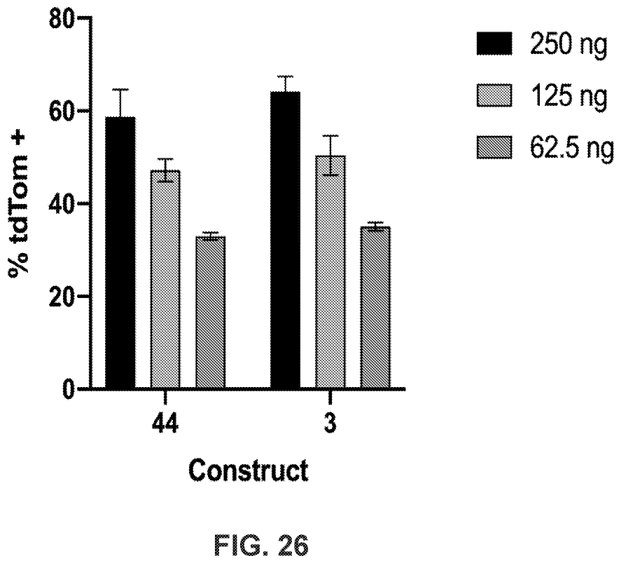

Construct 44 (configuration shown in FIG. 25, second from top) contains a Pol III promoter driving expression of guide scaffold 174 and spacer 12.7 in the reverse orientation of construct 3 (top configuration in FIG. 15). FIG. 26 demonstrates that construct 44, when delivered by nucleofection of an AAV transgene plasmid, modifies the target stop cassette in mNPCs similarly to construct 3 at in a dose-dependent manner.

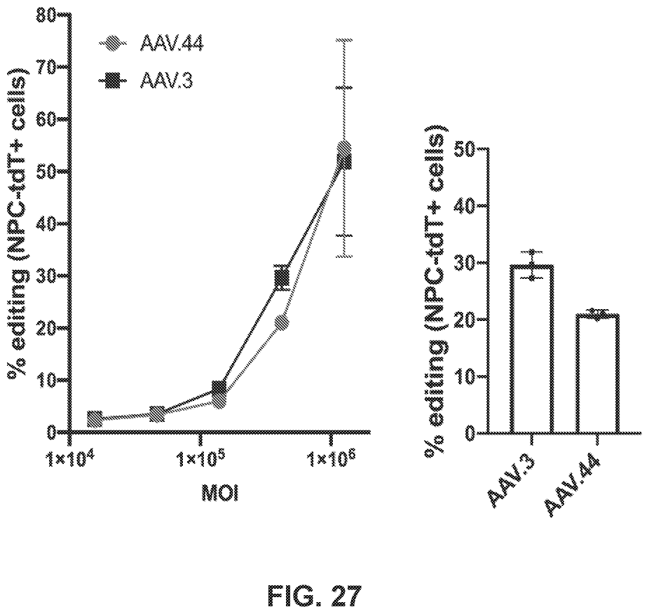

FIG. 27 shows that construct 44, delivered as an AAV vector, edits the target stop cassette in mNPCs, further supporting the utility of this construct. AAV.3 and AAV.44 were generated with transgene constructs 3 and 44, respectively. Each vector displayed dose-dependent editing at the target locus (FIG. 26, left panel, in which the vector was assayed using 3-fold dilutions). FIG. 27, right panel, shows editing results at an MOI of 3×10⁵, in which AAV.44 had 60% of the editing potency of the original configuration of vector AAV.3.

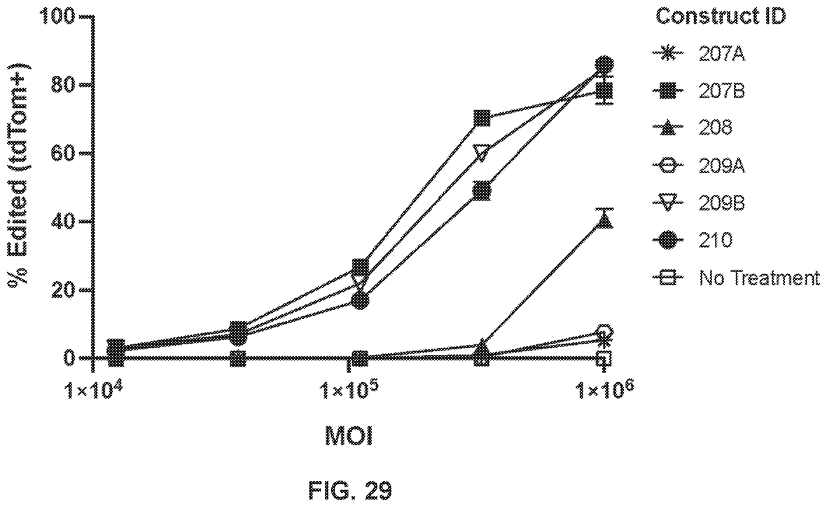

Additional configurations were explored, such that the gRNA transcriptional unit (Pol III U6 promoter driving the expression of the gRNA scaffold and indicated spacer) was placed either upstream or downstream of the CasX gene and was either in the forward or reverse orientation (FIG. 28). Table 14 lists the sequences of key AAV elements with varying positions and orientations of the gRNA promoter to drive gRNA expression, and Table 15 lists the full AAV transgene sequences within ITRs. The resulting AAV constructs were used to produce AAVs, which were used to transduce mNPCs to assess editing level at the tdTomato locus. The results of this experiment are illustrated in FIG. 29. The data demonstrate that AAVs produced from Constructs 207B, 209B, and 210 were able to induce similar levels of editing at the tdTomato locus in a dose-dependent manner. Meanwhile, the configuration used in Construct 208, where the U6-gRNA transcriptional unit was in the reverse orientation downstream of the CasX gene, appeared to adversely affect gene editing rate at the target locus.

TABLE 14
Sequences of key AAV elements with varying positions
and orientations of the gRNA transcriptional unit. “Rev
comp” denotes the reverse complementary sequence.

| | Component | SEQ | Length of |
|---|---|---|---|
| Construct ID | Name | ID NO: | Component (bp) |
| 207A/B, 208, | 5′ ITR | 3683 | 130 |
| 209A/B, 210 | UbC promoter | 3720 | 400 |
| | 5′ c-MYC NLS | 9290 | 27 |
| | CasX 491 | 9291 | 2931 |
| | 3′ c-MYC NLS | 9292 | 27 |
| | bGH poly(A) signal | 3696 | 209 |
| 207A/B, 209A/B | U6 promoter (Fwd) | 3698 | 242 |
| | Scaffold 235 (Fwd) | 3631 | 89 |
| 207B, 209B | Spacer 12.7 (Fwd) | 4049 | 21 |
| 207A, 209A | Spacer NT (Fwd) | 4057 | 18 |
| 208, 210 | U6 promoter | 3987 | 241 |
| | (Rev Comp) | | |
| | Scaffold 235 | 9293 | 99 |
| | (Rev Comp) | | |
| | Spacer 12.7 | 9294 | 20 |
| | (Rev Comp) | | |
| 207A/B, 208, | 3′ ITR | 3701 | 141 |
| 209A/B, 210 | | | |

TABLE 15
Sequences of AAV constructs within AAV ITRs.

| | Sequence | |
|---|---|---|
| Construct | (within AAV ITR) | Length of |
| ID | SEQ ID NO: | Construct (bp) |
| 207A | 9295 | 4352 |
| 207B | 9296 | 4352 |
| 208 | 9297 | 4344 |
| 209A | 9298 | 4354 |
| 209B | 9299 | 4354 |
| 210 | 9300 | 4364 |

The results of these experiments demonstrate that the orientation of parts within the AAV genome can be varied, yet result in sufficient expression of the CRISPR proteins and the guide RNA. This shows that specific orientations or positions of the regulatory elements relative to the encoded protein or RNA components may allow controlled modulation of expression in CasX-packaging AAV constructs that contain one or multiple guides.

### Example 8: Small CRISPR Protein Potency is Enhanced by Inclusion of Additional Regulatory Elements in the AAV Vector that are not Possible with a Larger Protein

Experiments were performed to demonstrate that transcriptional levels mediated by AAV vectors delivering small CRISPR proteins (such as CasX) can be enhanced by inclusion of different regulatory elements (intronic sequences, enhancers, etc.) that conventionally do not fit in AAV vectors expressing large transgene (e.g., spCas9) plasmids.

### Materials and Methods

Cloning and QC: A 4-part Golden Gate Assembly consisting of a pre-digested AAV backbone, small CRISPR protein-encoding DNA, and flanking 5′ and 3′ DNA sequences were used to generate AAV-cis plasmid as described in Example 1. 5′ sequences contained enhancer, protein promoter and N-terminal NLS, while 3′ sequences contained C-terminal NLS, WPRE, poly(A) signal, RNA promoter and guide RNA containing spacer 12.7. 5′ and 3′ parts were ordered as gene fragments, PCR-amplified, and assembled and assembled into AAV vectors. Cloning and plasmid QC, nucleofection, and FACS methods were conducted as described in Example 1.

Enhancement of editing by the inclusion of post-translation regulatory element (PTRE) 1, 2, or 3 in the AAV cis plasmid 3 was tested in combination with different promoters driving expression of CasX. A first set of promoters were tested; transgene plasmids 4, 35, 36 37, transgene plasmid 5, 38, 39, 40 and transgene plasmids 6, 42, 43 have the CasX protein expression driven by the CMV, UbC, EFS, CMV-s promoters, respectively. A second set of constructs tested included PTREs between the protein and poly(A) signal sequences and were generated with the Jet and JetUsp promoters compared to the UbC promoter (transgenes 58, 72, 73, 74; transgenes 59, 75, 76, 77 and transgenes 53, 80 and 81 respectively) driving expression of CasX. The PTRE sequences are listed in Table 16, and enhancer plus promoter sequences are listed in Table 17. The sequences of the additional components of the AAV constructs, with the exception of sequences encoding the CasX (Table 26) and the one or more gRNA (Tables 23 and 24), are listed in Table 45.

TABLE 16
Constructs and sequences of post-transcription elements (PTRE)
tested on base construct ID 4, 5, 6, 53, 58, and 59

| SEQ | | | Length of |
|---|---|---|---|
| ID NO: | Construct ID | PTRE | PTRE (bp) |
| 3615 | 35, 38, 72, 75 | 1 | 598 |
| 3616 | 36, 39, 42, 73, 76, 80 | 2 | 593 |
| 3617 | 37, 40, 43, 74, 77, 81 | 3 | 247 |

TABLE 17
Enhancer elements and sequences tested in
combination with the CMV core promoter

| SEQ | | | Core | Length of |
|---|---|---|---|---|
| ID NO: | ID | Enhancer | promoter | Promoter (bp) |
| 3618 | 3 | CMV | CMV | 584 |
| 3619 | 64 | N/A | CMV | 204 |
| 3620 | 65 | Syn 1 | CMV | 414 |
| 3621 | 66 | NPC5 | CMV | 314 |
| 3622 | 67 | NPC7 | CMV | 324 |
| 3623 | 68 | NPC127 | CMV | 304 |
| 3624 | 69 | NPC190 | CMV | 364 |
| 3625 | 70 | NPC249 | CMV | 274 |
| 3626 | 71 | NPC286 | CMV | 354 |

### Results

The effects of PTREs on transgene expression were assessed by cloning 3 enhancer sequences (PTRE1, PTRE2, and PTR3, Table 16) into an AAV-cis plasmid (construct 3) and construct plasmids containing shorter protein promoters (constructs 4, 5, 6, 53, 57 and 58 contain 400, 234, 335, 400, 164 and 326 bp promoter sequences, respectively).

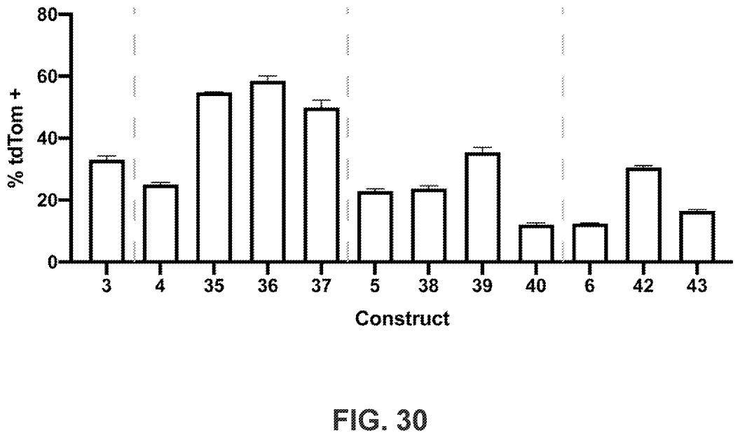

AAV-cis plasmid activity was first confirmed by nucleofection in mNPC-tdT cells. For each vector, addition of PTRE enhanced editing activity at various levels (FIG. 30). Table 18 provides the lengths of promoter and PTREs. The addition of PTRE2 to the transgene cassette showed the highest CasX editing activity enhancement, with a 2-fold increase in editing levels for construct 36 compared to construct 4 (58.5% vs 25%), a 1.5-fold increase for construct 39 (35.4% vs 22.9%) compared to construct 5 and a 3-fold increase for construct 42 compared to construct 6 (30.5% vs 12%). The shortest enhancer sequence, PTRE3, also increased protein activity at various levels among construct 37 and 43 compared to other vectors.

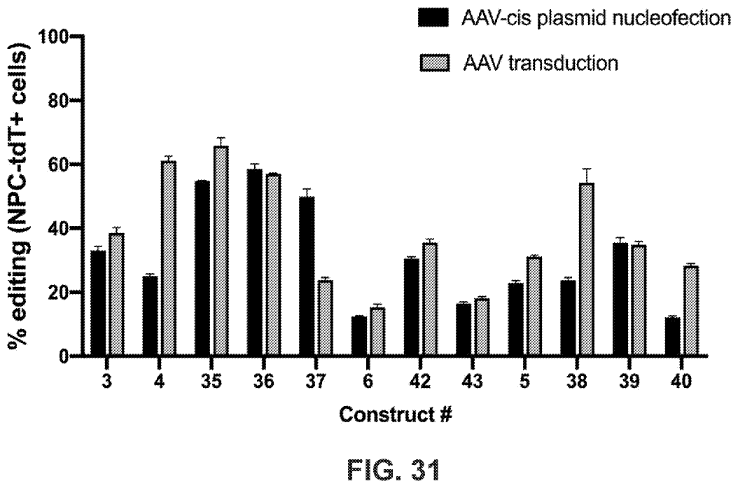

Improvements in editing levels were also observed when constructs were packaged into AAV. Inclusion of PTRE2 in transgene increased editing across the AAV vectors in a similar manner. Trends in on-target editing observed in mNPCs with the AAV infection generally correlated with the AAV plasmid nucleofection data set (FIG. 31).

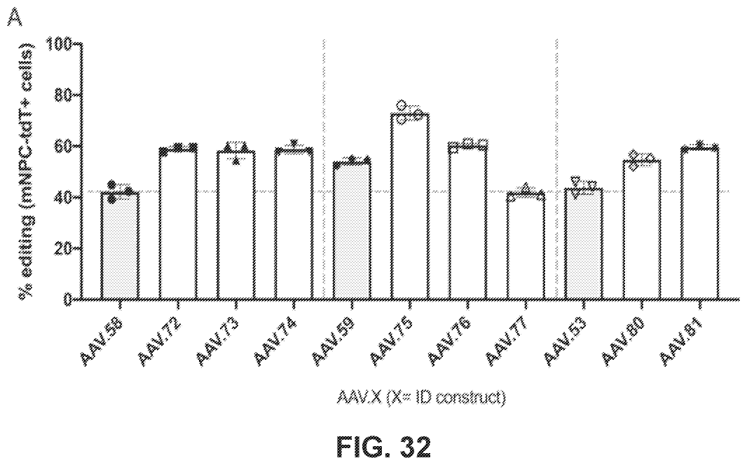

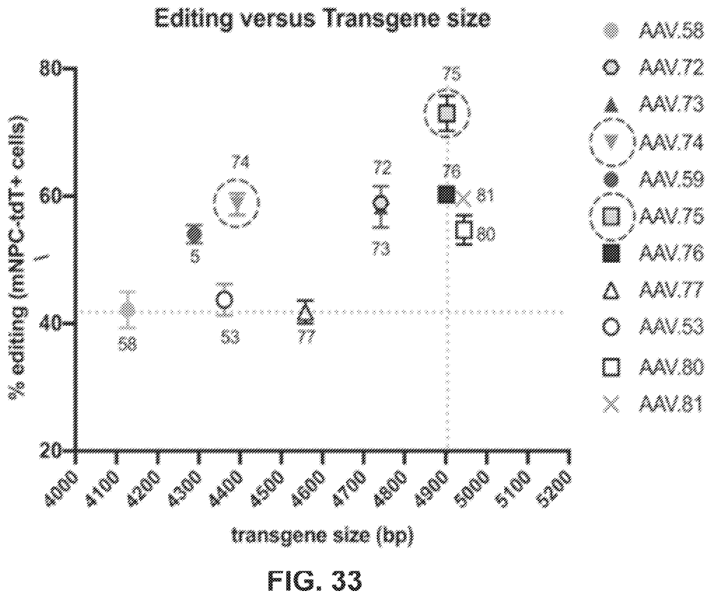

The trend was confirmed by testing another set of promoters with inclusion of these enhancer sequences. Across all AAV vectors tested, constructs including PTRE1 or PTRE2 in genomes yielded an average 1.5-fold increase compared to base vectors (FIG. 32). Unique combinations of short promoter and these post-transcriptional sequences led to the identification of vectors with increased editing levels with shorter promoters (e.g., AAV.74), which represents an advantage both for AAV manufacturing being under the carrying capacity limit of AAV, and allows for inclusion of more regulatory elements and CRISPR elements (e.g., additional guides). Comparisons of editing versus transgene size are plotted in FIG. 33.

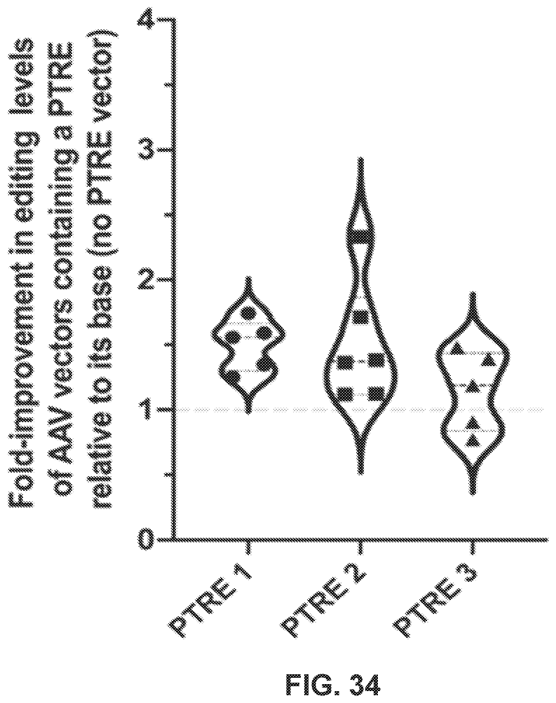

The results also demonstrate that inclusion of PTRE1 in the transgene plasmid improved editing levels across all promoters evaluated (FIG. 34), with less variability, while PTRE2 yielded the highest transgene improvement but with more variability across the promoters tested.

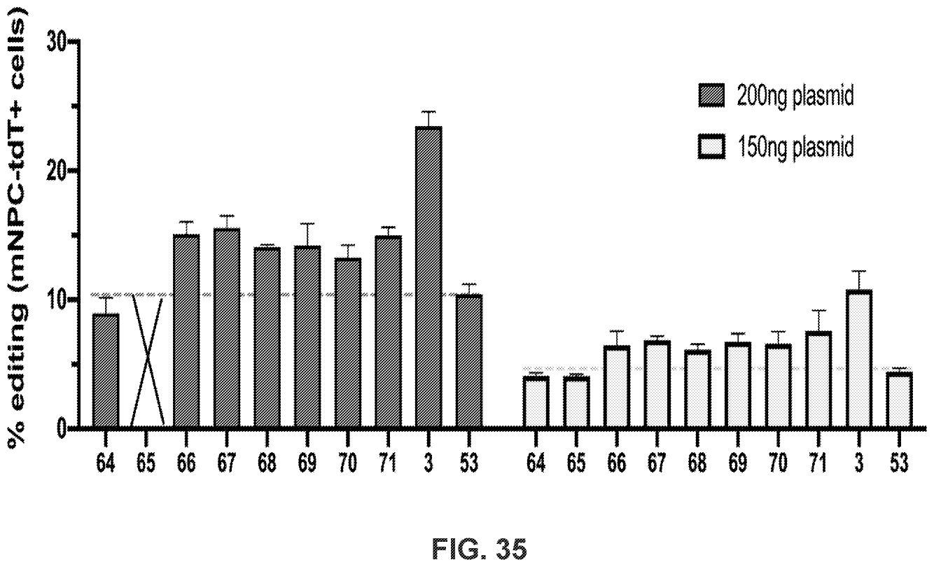

Several constructs with tissue-specific neuronal enhancers upstream of a single constitutive promoter were also tested. In this assay, 7 neuronal enhancer sequences (constructs 65-72) were cloned into a single AAV-cis plasmid (construct 64) harboring a core CMV promoter and all demonstrated improved editing via nucleofection over base construct 64 (FIG. 35). These constructs also outperformed construct 53, which contains a UbC promoter but did not outperform construct 3 which harbors the full CMV promoter (CMV enhancer+CMV core promoter).

TABLE 18
Constructs with or without PTREs and indicated sequence lengths
AAV construct
(Sequence length indicated below)

| | 3 | 4 | 35 | 36 | 37 | 5 | 38 | 39 | 40 | 6 | 42 | 43 |
|---|---|---|---|---|---|---|---|---|---|---|---|---|
| Promoter | 584 | 400 | | | | 234 | | | | 335 | | |
| Length | | | | | | | | | | | | |
| PTRE 1 | — | — | 592 | — | — | — | 592 | — | — | — | — | — |
| PTRE 2 | — | — | — | 593 | — | — | — | 593 | — | — | 593 | — |
| PTRE 3 | — | — | — | — | 247 | — | — | — | 247 | — | — | 247 |
| AAV.transgene | 4550 | 4349 | 4964 | 4965 | 4619 | 4183 | 4798 | 4799 | 4453 | 4284 | 4900 | 4554 |

The results demonstrate that use of small promoters in the AAV transgene constructs permits the inclusion of additional accessory elements. These additional accessory elements, such as post-transcriptional regulatory elements to AAV-transgenes expressing CasX under the control of short but strong promoter sequences enable increased CasX expression and on-target editing while reducing cargo size such that all components can be incorporated into a single AAV vector.

### Example 9: Demonstration that a CasX:Dual-gRNA System Expressed from a Single AAV Vector can Edit the Target Locus In Vitro

Experiments were performed to demonstrate the following: 1) CasX and dual gRNAs expressed from an all-in-one AAV vector can edit the target locus; 2) the ability to package and deliver CasX with a dual-guide system within a single AAV vector for targeted editing; and 3) editing of a therapeutically-relevant locus by CasX and dual gRNAs delivered via a single AAV vector can excise the targeted genomic region. For the editing at a therapeutically-relevant locus by the CasX-dual-gRNA system, experiments were conducted to demonstrate the ability of CasX and the dual-guide system to mediate excision of a CTG repeat in the 3′UTR region of the human DMPK gene when delivered via AAVs in vitro into HEK293T cells. The ability to demonstrate editing mediated by the CasX:dual-gRNA system delivered and expressed from a single all-in-one AAV vector is significant because this is not achievable with traditionally used Cas9-based systems.

### Materials and Methods

AAV plasmid cloning and nucleofection were conducted as described in Example 1.

Various configurations of two gRNA transcriptional unit blocks, also referred as “guide RNA stacks”, of the AAV transgene are illustrated in FIGS. 38-39 and FIG. 75.

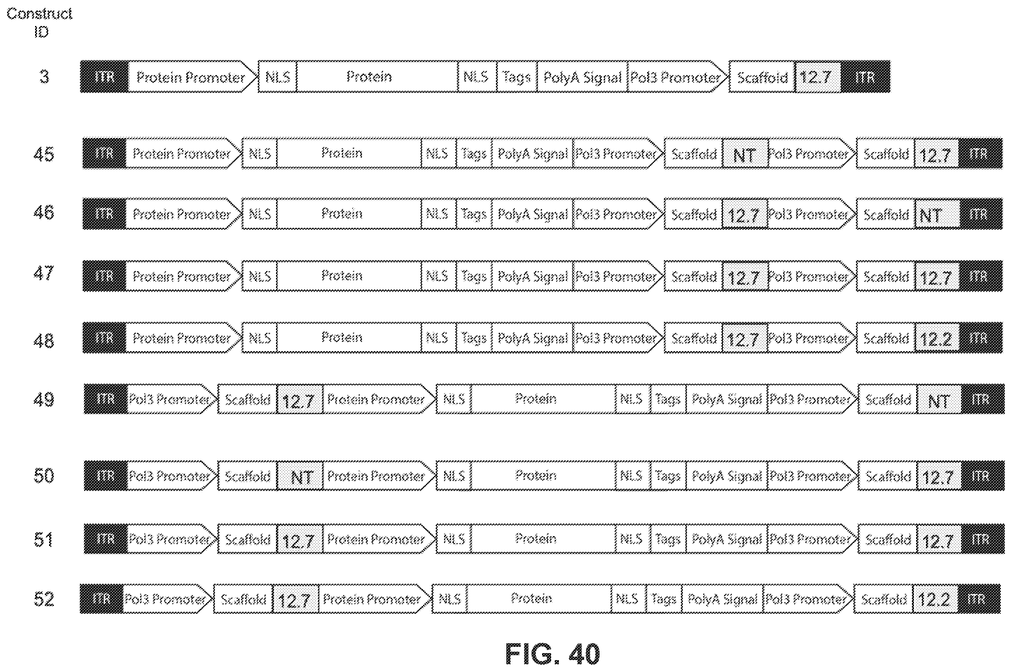

FIG. 40 illustrates the configurations of the dual-guide stacks, with each stack composed of a gRNA scaffold-spacer combination 174.12.7, 174.12.2 or 174.NT driven by the human U6 promoter listed in Table 8. These specific dual-guide stacks were investigated by cloning two gRNA stacks in a tail-to-tail orientation (Construct ID 45-49) on the 3′ end of the poly(A) or in the same transcriptional orientation as the protein promoter-CasX unit, one on each side of the CasX unit (Construct ID 50-52). Pentagon-shaped boxes for CasX protein promoter and Pol III gRNA promoter depict orientation of transcription (tapered point; 5′ to 3′ or 3′ to 5′ orientation). Spacer sequences are 12.2 (TATAGCATACATTATACGAA; SEQ ID NO: 4056)); 12.7 (CTGCATTCTAGTTGTGGTTT; SEQ ID NO: 4049); and NT (GGGTCTTCGAGAAGACCC; SEQ ID NO: 4057).

AAV vector production and titering were conducted as described in Example 1. AAV transduction and editing assessment via FACs sorting were conducted as described in Example 1.

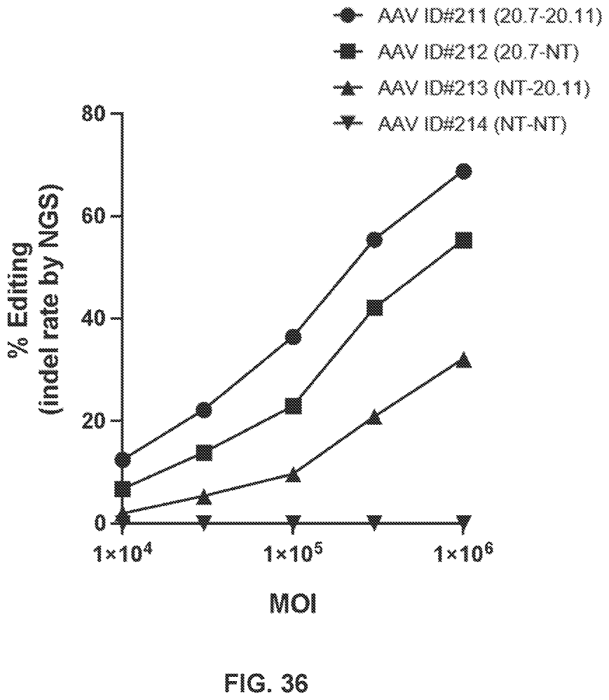

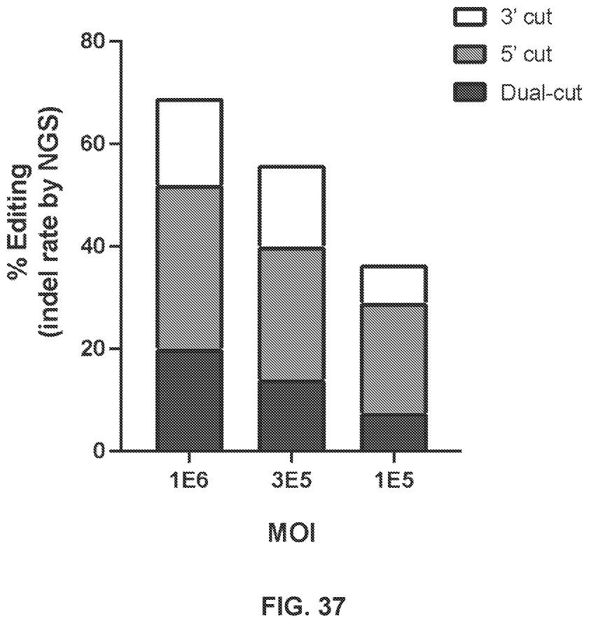

AAV constructs (Construct ID 211-214) assessed in FIG. 36 and FIG. 37 were generated using methods described in Example 1. Sequences for these AAV plasmids are listed in Table 19.

TABLE 19
Sequences of AAV constructs with dual-guides targeting either side of the CTG
repeat in DMPK 3' UTR. NT=non-targeting guide*

| | | DNA sequence | Length of |
|---|---|---|---|
| Construct ID | Component Name | or SEQ ID NO: | Component (bp) |
| 211 through | 5′ ITR | 3683 | 130 |
| 214 | buffer sequence | 3684 | 23 |
| | CMV enhancer + promoter | 9301 | 584 |
| | buffer sequence | 9302 | 18 |
| | Kozak | GCCACC | 6 |
| | start codon | ATGGCC | 6 |
| | SV40 NLS | 9305 | 21 |
| | linker | TCTAGA | 6 |
| | CasX 491 | 9291 | 2931 |
| | linker | GGATCC | 6 |
| | SV40 NLS | 9308 | 21 |
| | HA tag | 9309 | 27 |
| | linker + stop codon | GGATCCTAA | 9 |
| | buffer sequence | 3695 | 30 |
| | bGH poly(A) signal | 3696 | 209 |
| | buffer sequence | GGTACCGT | 8 |
| | U6 promoter | 3698 | 242 |
| | buffer sequence | GAAACACC | 8 |
| | Scaffold 174 | 9311 | 89 |
| | Spacer 1 | See specific dual guide combos below | |
| | (5′ CTG) | | |
| | buffer sequence | 9312 | 20 |
| | U6 promoter | 3698 | 242 |
| | buffer sequence | GAAACACC | 8 |
| | Scaffold 174 | 9311 | 89 |
| | Spacer 2 | See specific dual guide combos below | |
| | (3′ CTG) | | |
| | buffer sequence | 3700 | 17 |
| | 3' ITR | 3701 | 141 |
| 211 | Spacer 1 (20.7) | 9312 | 20 |
| | Spacer 2 (20.11) | 9313 | 20 |
| 212 | Spacer 1 (20.7) | 9312 | 20 |
| | Spacer 2 (NT) | 9314 | 18 |
| 213 | Spacer 1 (NT) | 9314 | 18 |
| | Spacer 2 (20.11) | 9313 | 20 |
| 214 | Spacer 1 (NT) | 9314 | 18 |
| | Spacer 2 (NT) | 9314 | 18 |

*Components are listed in a 5' to 3' order within the constructs

Production of AAV vectors from AAV constructs 211-214 and subsequent titering were performed as described in Example 1.

### AAV Transduction of HEK293T Cells:

˜10,000 ITEK293T cells per well were seeded in 96-well plates. 24 hours later, seeded cells were treated with AAVs encoding CasX variant 491 with the dual-guide system (i.e., scaffold 174 with spacers 20.7-20.11, 20.7-NT, NT-20.11, or NT-NT; refer to Table 19 for sequences). Viral infection conditions were performed in triplicate, with normalized number of viral genomes (vg) among experimental vectors, in a series of three-fold dilution of multiplicity of infection (MOI) ranging from ˜1E6 to 1E4 vg/cell. Five days post-transduction, AAV-treated ITEK293T cells were harvested for gDNA extraction for editing analysis at the DMPK locus by next generation sequencing (NGS). Briefly, amplicons were amplified from 200 ng of extracted gDNA with a set of primers targeting the CTG repeat region in the DMPK 3′ UTR and processed as described in Example 18.

### Results

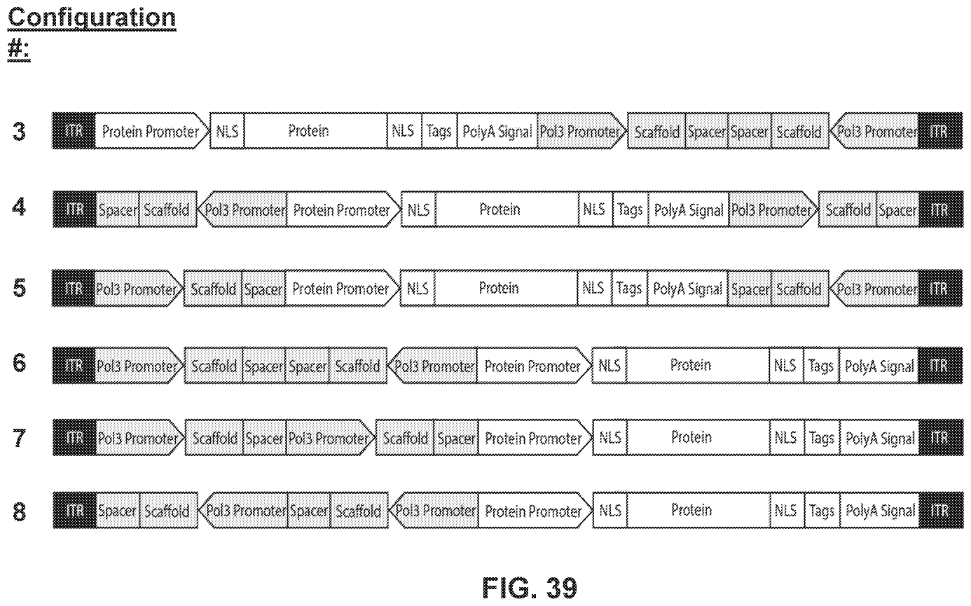

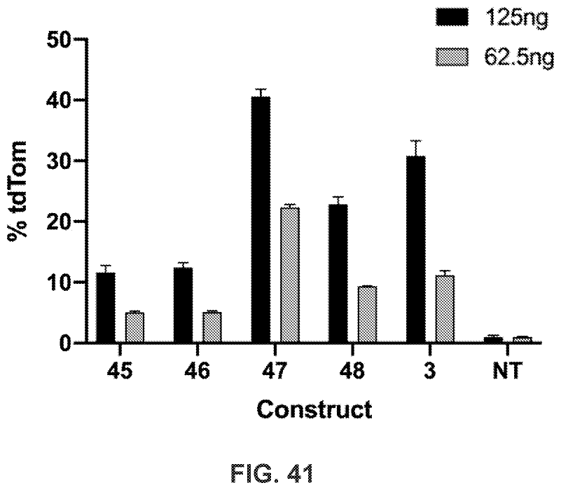

FIG. 38 is a schematic of two AAV construct configurations (architecture 1 and architecture 2). FIG. 39 and FIG. 75 illustrate additional AAV construct configurations, while FIG. 40 depicts the specific dual-spacer combinations. The results of the editing assay portrayed in FIG. 41 demonstrate that the constructs delivered as AAV transgene plasmids to mNPCs in architecture 2 were able to edit with enhanced potency. The results from the assay assessing the different combinations of targeting and non-targeting spacers demonstrate that each individual gRNA was active, although, architectures with one targeting spacer and one non-targeting spacer (constructs 45 and 46) yielded approximately 18% lower editing levels. Certain combinations of targeting spacers yielded increased efficacy. While use of the dual-spacer combination 12.7-12.2 (construct 48) resulted in editing with significant potency, use of two sets of 12.7 spacers (construct 47) resulted in editing with 10% greater potency than that seen with the single gRNA architecture (construct 3) (FIG. 41).

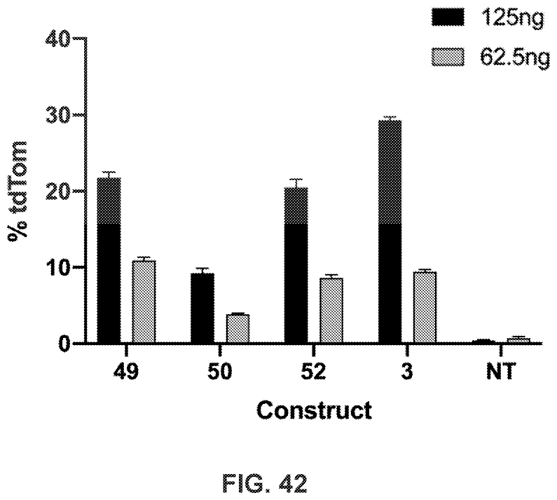

The bar plot in FIG. 42 shows the results that use of AAV constructs 49, 50, and 52, which had the arrangements where two gRNA transcriptional units were placed on either side of the CasX gene, were also able to edit the target nucleic acid when delivered to mNPCs.

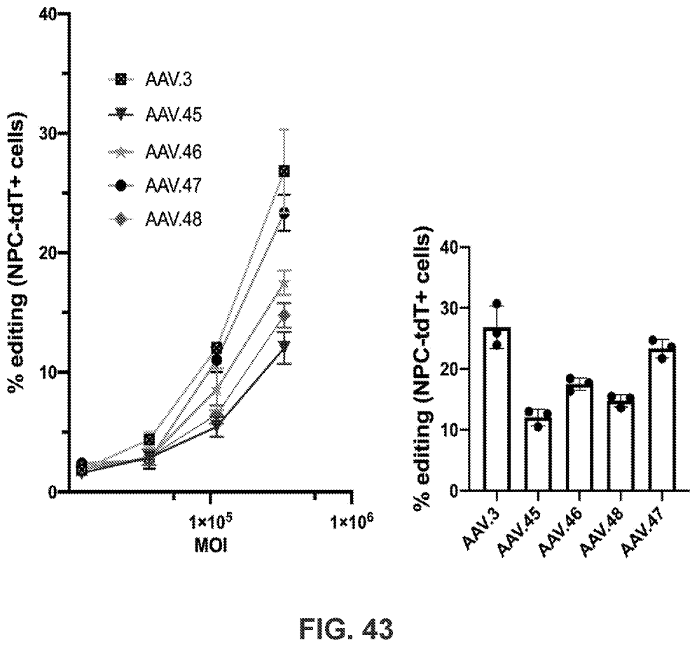

The bar plot in FIG. 43 shows that use of AAV constructs 3, 45, 46, 47, and 48, delivered as AAVs, were able to edit the target stop cassette in mNPCs. Each vector displayed dose-dependent editing at the target locus (FIG. 43, left panel). At an MOI of 3E5, AAV.47 had <5% less potency than the level observed with the original orientation vector AAV.3 (FIG. 43, right panel).

Experiments were also performed to demonstrate the use of CasX and a dual-guide system in targeting and excising the CTG repeat in the 3′UTR region of the human DMPK gene. The significance of evaluating the ability to target this repeat is that the neuromuscular disease myotonic dystrophy type 1 (DM1) is caused by the abnormal CTG repeat expansion in the 3′ noncoding region of the human DMPK gene. Here, HEK293T cells were transduced with dual-guide AAVs harboring either two DMPK-targeting spacers (20.7 and 20.11), the combination of one DMPK-targeting spacer and one non-targeting (NT) spacer (20.7 and NT or NT and 20.11), or two non-targeting spacers (NT-NT) at various MOIs. The results shown in FIG. 36 demonstrate on-target editing at either side or both sides flanking the CTG repeat expansion in transduced HEK293T cells occurred in a dose-dependent manner. The highest level of indel rate was attained with the dual-guide AAV (spacers 20.7 and 20.11), reaching ˜70% editing efficiency at the highest MOI of 1E6 vg/cell. In addition, infecting cells with AAVs expressing the combination of one DMPK-targeting spacer and one NT spacer (20.7 and NT or NT and 20.11) revealed that a higher editing efficiency was achieved on the 5′ region (by spacer 20.7 and NT) of the CTG repeat in comparison to editing on the 3′ region (by spacer NT and 20.11) (FIG. 36). FIG. 37 illustrates the quantification of percent editing of indel rate detected by NGS for the various types of editing (i.e., editing at 5′ or 3′ of CTG repeat, or dual-editing resulting in dropout of CTG repeat) induced by the AAVs harboring two DMPK-targeting spacers (20.7-20.11). Double-cut editing resulting in CTG repeat excision occurred in a dose-dependent manner, with 21% excision rate achieved at the highest MOI of 1E6 (FIG. 37). High levels of editing were similarly observed at the individual 5′ or 3′ region of the CTG repeat, with a majority of indel events occurring in the 5′ region.

Altogether, these experiments demonstrate the feasibility of using dual gRNAs in combination with the full CasX protein sequence in a single AAV, which would not be achievable with the use of larger CRISPR proteins, such as Cas9, due to the transgene packaging constraints of the AAV capsid. The experiments also show that dual guide RNAs in an all-in-one vector construct were able to retain the ability to edit the target nucleic acid. Furthermore, the results demonstrate the ability to package and deliver CasX with the dual-guide system from an all-in-one single AAV vector in vitro, which resulted in efficient editing and excision of the target genomic region. In addition to using a dual-guide system to excise a target genomic region, combining two gRNA transcriptional units could also provide the ability to 1) increase gRNA expression and thus CasX-mediated editing or 2) target two distinct genes that might have cooperative therapeutic effects. The effects of varying the orientation and position of gRNA promoters are further investigated in Examples 31 and 32.

### Example 10: Nuclear Localization Sequence (NLS) Selection Enhances Small CRISPR Protein Potency

Experiments were conducted to determine whether alteration of the nuclear localization sequence (NLS) utilized in constructs can modulate editing.

### Materials and Methods

AAV vectors were cloned and produced according to standard methods, which are described in Example 1. The amino acid sequences of the encoded NLS are presented in Tables 20 and 21.

Methods for production of AAV vectors and nucleofection were conducted as described in Example 1. The sequences of the additional components of AAV constructs, with the exception of sequences encoding the CasX (Table 26) and the one or more gRNA (Tables 23 and 24), are listed in Table 45.

TABLE 20
N-terminal NLS sequences

| SEQ ID | | |
|---|---|---|
| NO: | NLS Amino Acid Sequence* | NLS ID |
| 3411 | PKKKRKVSR | 1 |
| 3412 | PKKKRKVGGSPKKKRKVGGSPKKKRKVGGSPKKKRKVSR | 2 |
| 3413 | PKKKRKVGGSPKKKRKVGGSPKKKRKVGGSPKKKRKVGGSPKKKRKVGGSPKKKRKVSR | 3 |
| 3414 | PAAKRVKLDSR | 4 |
| 3415 | PAAKRVKLDGGSPAAKRVKLDSR | 5 |
| 3416 | PAAKRVKLDGGSPAAKRVKLDGGSPAAKRVKLDGGSPAAKRVKLDSR | 6 |
| 3417 | PAAKRVKLDGGSPAAKRVKLDGGSPAAKRVKLDGGSPAAKRVKLDGGSPAAKRVKLDGGSPA | 7 |
| | AKRVKLDSR | |
| 3418 | KRPAATKKAGQAKKKKSR | 8 |
| 3419 | KRPAATKKAGQAKKKKGGSKRPAATKKAGQAKKKKSR | 9 |
| 3420 | PAAKRVKLDGGSPKKKRKVSR | 10 |
| 3421 | PAAKKKKLDGGSPKKKRKVSR | 11 |
| 3422 | PAAKKKKLDSR | 12 |
| 3423 | PAAKKKKLDGGSPAAKKKKLDGGSPAAKKKKLDSR | 13 |
| 3424 | PAAKKKKLDGGSPAAKKKKLDGGSPAAKKKKLDGGSPAAKKKKLDSR | 14 |
| 3425 | PAKRARRGYKCSR | 15 |
| 3426 | PAKRARRGYKCGSPAKRARRGYKCSR | 16 |
| 3427 | PRRKREESR | 17 |
| 3428 | PYRGRKESR | 18 |
| 3429 | PLRKRPRRSR | 19 |
| 3430 | PLRKRPRRGSPLRKRPRRSR | 20 |
| 3431 | PAAKRVKLDGGKRTADGSEFESPKKKRKVGGS | 21 |
| 3432 | PAAKRVKLDGGKRTADGSEFESPKKKRKVPPPPG | 22 |
| 3433 | PAAKRVKLDGGKRTADGSEFESPKKKRKVGIHGVPAAPG | 23 |
| 3434 | PAAKRVKLDGGKRTADGSEFESPKKKRKVGGGSGGGSPG | 24 |
| 3435 | PAAKRVKLDGGKRTADGSEFESPKKKRKVPGGGSGGGSPG | 25 |
| 3436 | PAAKRVKLDGGKRTADGSEFESPKKKRKVAEAAAKEAAAKEAAAKAPG | 26 |
| 3437 | PAAKRVKLDGGKRTADGSEFESPKKKRKVPG | 27 |
| 3438 | PAAKRVKLDGGSPKKKRKVGGS | 28 |
| 3439 | PAAKRVKLDPPPPKKKRKVPG | 29 |
| 3440 | PAAKRVKLDPG | 30 |
| 3441 | PAAKRVKLDGGGSGGGSGGGS | 31 |
| 3442 | PAAKRVKLDPPP | 32 |
| 3443 | PAAKRVKLDGGGSGGGSGGGSPPP | 33 |
| 3444 | PKKKRKVPPP | 34 |
| 3445 | PKKKRKVGGS | 35 |

*Sequences in bold are NLS, while unbolded sequences are linkers.

TABLE 21
C-terminal NLS sequences

| SEQ ID | | |
|---|---|---|
| NO: | NLS Amino Acid Sequence* | NLS ID |
| 3446 | GSPKKKRKV | 1 |
| 3447 | GSPKKKRKVGGSPKKKRKVGGSPKKKRKVGGSPKKKRKV | 2 |
| 3448 | GSPKKKRKVGGSPKKKRKVGGSPKKKRKVGGSPKKKRKVGGSPKKKRKVGGSPKKKRKV | 3 |
| 3449 | GSPAAKRVKLD | 4 |
| 3450 | GSPAAKRVKLDGGSPAAKRVKLD | 5 |
| 3451 | GSPAAKRVKLDGGSPAAKRVKLDGGSPAAKRVKLDGGSPAAKRVKLD | 6 |
| 3452 | GSPAAKRVKLDGGSPAAKRVKLDGGSPAAKRVKLDGGSPAAKRVKLDGGSPAAKRVKLDGG | 7 |
| | SPAAKRVKLD | |
| 3453 | GSKRPAATKKAGQAKKKK | 8 |
| 3454 | KRPAATKKAGQAKKKKGGSKRPAATKKAGQAKKKK | 9 |
| 3455 | GSPAAKRVKLGGSPAAKRVKLGGSPKKKRKVGGSPKKKRKV | 10 |
| 3456 | GSKLGPRKATGRWGS | 11 |
| 3457 | GSKRKGSPERGERKRHWGS | 12 |
| 3458 | GSPKKKRKVGSGSKRPAATKKAGQAKKKKLE | 13 |
| 3459 | GPKRTADSQHSTPPKTKRKVEFEPKKKRKV | 14 |
| 3460 | GGGSGGGSKRTADSQHSTPPKTKRKVEFEPKKKRKV | 15 |
| 3461 | AEAAAKEAAAKEAAAKAKRTADSQHSTPPKTKRKVEFEPKKKRKV | 16 |
| 3462 | GPPKKKRKVGGSKRTADSQHSTPPKTKRKVEFEPKKKRKV | 17 |
| 3463 | GPAEAAAKEAAAKEAAAKAPAAKRVKLD | 18 |
| 3464 | GPGGGSGGGSGGGSPAAKRVKLD | 19 |
| 3465 | GPPKKKRKVPPPPAAKRVKLD | 20 |
| 3466 | GPPAAKRVKLD | 21 |
| 3467 | GSPKKKRKV | 22 |
| 3468 | GSPAAKRVKLD | 23 |
| 3469 | VGSKRPAATKKAGQAKKKK | 24 |
| 3470 | TGGGPGGGAAAGSGSPKKKRKVGSGSKRPAATKKAGQAKKKKLE | 25 |
| 3471 | TGGGPGGGAAAGSGSPKKKRKVGSGSKRPAATKKAGQAKKKKLE | 26 |
| 3472 | TGGGPGGGAAAGSGSPKKKRKVGSGS | 27 |
| 3473 | PPPPKKKRKVPPP | 28 |
| 3474 | GGSPKKKRKVPPP | 29 |
| 3475 | PPPPKKKRKV | 30 |
| 3476 | GGSPKKKRKV | 31 |
| 3477 | GGSPKKKRKVGGSGGSGGS | 32 |
| 3478 | GGSPKKKRKVGGSPKKKRKV | 33 |
| 3479 | GGSGGSGGSPKKKRKVGGSPKKKRKV | 34 |
| 3480 | VGGGSGGGSGGGSPAAKRVKLD | 35 |
| 3481 | VPPPPAAKRVKLD | 36 |
| 3482 | VPPPGGGSGGGSGGGSPAAKRVKLD | 37 |
| 3483 | VGGGSGGGSGGGSPAAKRVKLD | 38 |
| 3484 | VPPPPAAKRVKLD | 39 |
| 3485 | VPPPGGGSGGGSGGGSPAAKRVKLD | 40 |
| 3486 | VGSPAAKRVKLD | 41 |

*Sequences in bold are NLS, while unbolded sequences are linkers.

AAV transduction and editing level assessment in mNPTC-tdT cells by FACS were conducted as described in Example 1.

### Results

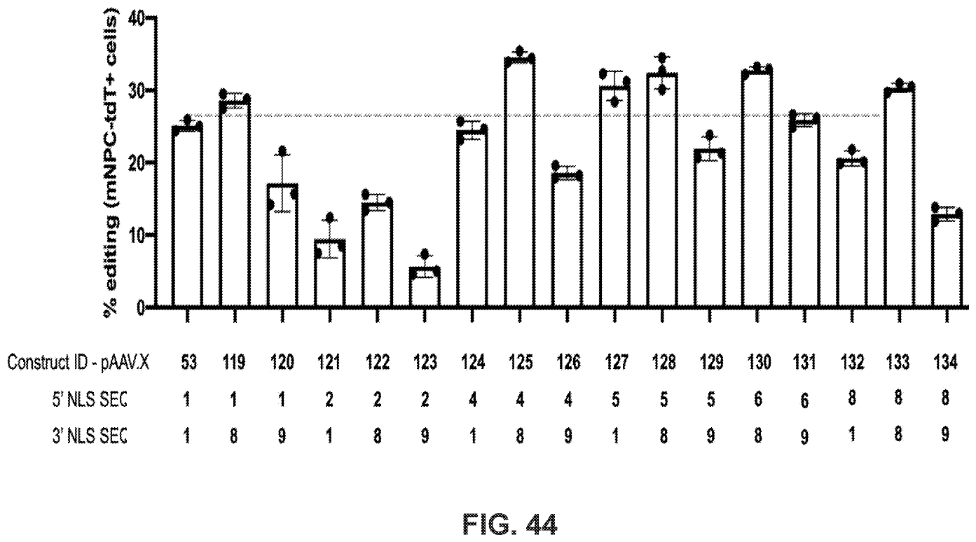

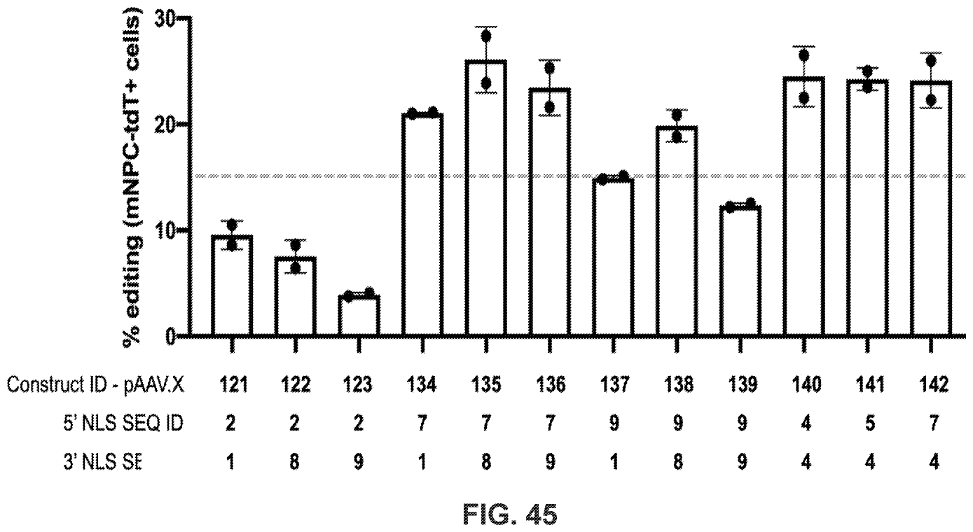

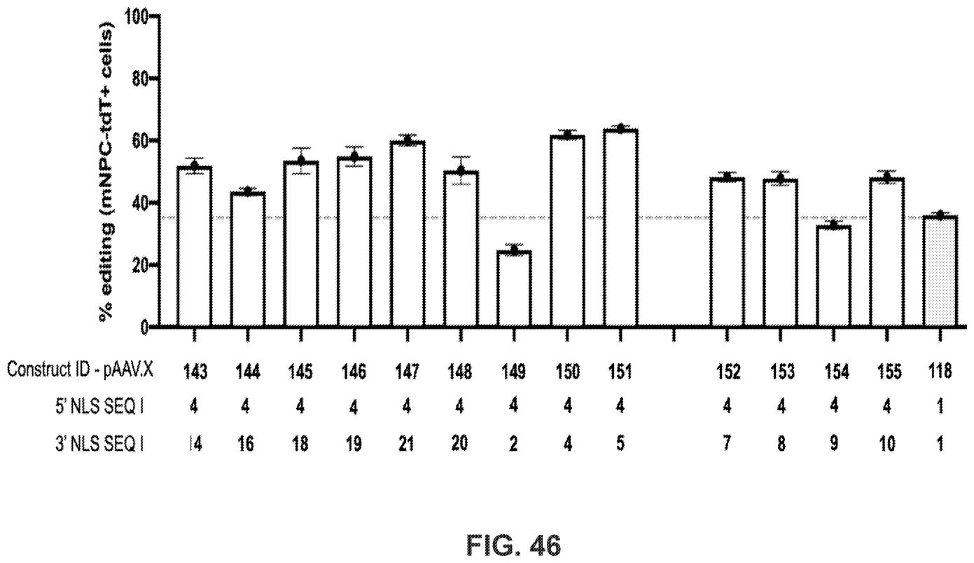

Initial plasmid nucleofection revealed that a number of NLS permutations displayed improved editing when compared to control (1×SV40 NLS on both the N- and C-termini). In particular, N-terminal variants containing c-MYC or Nucleoplasmin NLSs significantly outperformed SV40 NLS combinations (FIG. 44). This trend in N-terminal NLS variation was replicated in AAV transduction, where c-MYC and Nucleoplasmin NLS variants again outperformed SV40 NLS variants (FIG. 45). Finally, variations holding the c-MYC constant (FIG. 46) were tested, and the results demonstrated that the constructs with the highest level of editing contained a c-MYC NLS on both the N- and C-terminus.

The data show that selecting the amino acid sequence of the NLS can enhance editing outcomes in the AAV setting. Specifically, N-terminal Cmyc-containing NLS variants showed a clear improvement compared to N-terminal SV40 NLS variants. In addition, C-terminal c-MYC and Nucleoplasmin variants improve editing over SV40 NLS variants. Repetitions of the SV40 NLS seem to be deleterious for editing efficiency on both the N- and C-terminals.

### Example 11: Introns in the 5′ UTR can Enhance Small CRISPR Protein Expression

An experiment is performed to test whether transcriptional levels mediated by AAV vectors delivering small CRISPR proteins (such as CasX) can be enhanced by inclusion of different regulatory elements such as intronic sequences taken from viral, mouse, or human genomes that conventionally do not fit in AAV vectors expressing large transgene (e.g., spCas9) plasmids.

### Methods

AAV cloning and production are as described in Example 1. 5′ sequences used to generate the AAV cis plasmid contain protein promoters including UbC, JeT, CMV, CAG, CBH, hSyn, or other Pol2 promoter, intronic region, and N-terminal NLS, while 3′ sequences contain C-terminal NLS, poly A signal, RNA promoter and guide RNA containing spacer 12.7. Non-limiting examples of intron sequences to be incorporated into the constructs are listed in Table 22.

Enhancement in editing by the inclusion of intron 36 (transgene plasmid 59) is tested against transgene plasmid 58, which was the baseline construct not containing the intron. The rest of the introns in Table 22 have been derived from viral, mouse, and human origin.

TABLE 22
Intron sequences for incorporation into base construct 58

| | SEQ | Length of |
|---|---|---|
| Intron | ID NO | intron (bp) |
| 1 | 3487 | 54 |
| 2 | 3488 | 67 |
| 3 | 3489 | 62 |
| 4 | 3490 | 49 |
| 5 | 3491 | 59 |
| 6 | 3492 | 67 |
| 7 | 3493 | 66 |
| 8 | 3494 | 86 |
| 9 | 3495 | 67 |
| 10 | 3496 | 70 |
| 11 | 3497 | 476 |
| 12 | 3498 | 69 |
| 13 | 3499 | 69 |
| 14 | 3500 | 70 |
| 15 | 3501 | 70 |
| 16 | 3502 | 108 |
| 17 | 3503 | 69 |
| 18 | 3504 | 206 |
| 19 | 3505 | 70 |
| 20 | 3506 | 68 |
| 21 | 3507 | 299 |
| 22 | 3508 | 226 |
| 23 | 3509 | 71 |
| 24 | 3510 | 69 |
| 25 | 3511 | 87 |
| 26 | 3512 | 84 |
| 27 | 3513 | 82 |
| 28 | 3514 | 66 |
| 29 | 3515 | 65 |
| 30 | 3516 | 66 |
| 31 | 3517 | 106 |
| 32 | 3518 | 69 |
| 33 | 3519 | 68 |
| 34 | 3520 | 68 |
| 35 | 3521 | 97 |
| 36 | 3522 | 140 |
| 37 | 3523 | 133 |
| 38 | 3524 | 190 |
| 39 | 3525 | 271 |
| 40 | 3526 | 96 |
| 41 | 3527 | 110 |
| 42 | 2528 | 270 |
| 43 | 3529 | 116 |
| 44 | 3530 | 67 |
| 45 | 3531 | 66# |

### Results

The effects of introns on transgene expression are assessed by cloning 50 different introns into AAV-cis plasmid and then assaying for editing in the tdTomato assay used in the Examples supra.

When compared to the base construct without an intron, the addition of an intronic sequence generally increases the overall editing efficiency of AAV transgenes.

The results are expected to support that the addition of introns to AAV-transgenes expressing CasX under the control of short but strong promoter sequences enables increased CasX expression and on-target editing while reducing cargo size, further optimizing the AAV system.

### Example 12: Improved Guide Variants Demonstrate Enhanced On-Target Activity In Vitro

Experiments were conducted to identify engineered guide RNA variants with increased activity at different genomic targets, including the therapeutically-relevant mouse and human RHO exon 1. Previous assays identified many different “hotspot” regions (e.g., stem loop) within the scaffold sequences holding the potential to significantly increase editing efficiency as well as specificity. Additionally, screens were conducted to identify scaffold variants that would increase the overall activity of the tested CRISPR system in an AAV vector across multiple different PAM-spacer combinations, without triggering off-target or non-specific editing. Achieving increased editing efficiency compared to current benchmark vectors would allow reduced viral vector doses to be used in in vivo studies, improving the safety of AAV-mediated CasX-guide systems.

### Methods

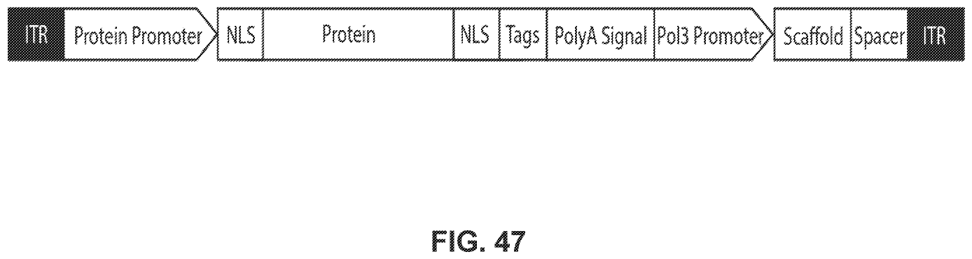

New gRNA scaffold and spacer variants were inserted into an AAV transgene construct for plasmid and viral vector validation (encoding sequences in Tables 23 and 24). CasX 491 variant protein was used for all constructs evaluated in this experiment, however the disclosure contemplates utilizing any of the CasX variants, including those of Table 5 and the encoding sequences of Table 26. The AAV transgene was conceptually broken up between ITRs into different parts, which consisted of the therapeutic cargo and accessory elements relevant to expression in mammalian cells and the nuclease-guide RNA complex (protein nuclease, scaffold, spacer). A schematic with its conceptual parts is shown in FIG. 47. The nucleic acid sequences of the remaining components common to the various constructs are presented in Table 45, the encoding sequences of the guides are presented in Tables 23 and 24, and the encoding sequences of the CasX are presented in Table 26 such that the various permutations of the transgene can be elucidated.

### Cloning:

Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. New AAV constructs were then transformed into chemically competent E. coli (Turbos or Stbl3s). Transformed cells were recovered for 1 hour in a 37° C. shaking incubator then plated on Kanamycin LB-Agar plates and allowed to grow at 37° C. for 12-16 hours. Colonies were picked into 6 mL of 2×yt treated with Kanamycin and allowed to grow for 7-14 hours, then mini-prepped and Sanger sequenced. The transformation and miniprep protocol were then repeated and spacer-cloned vectors were sequence verified again. Validated constructs were maxi-prepped. To assess the quality of maxi-preps, constructs were processed in two separate digests with XmaI (which cuts at several sites in each of the ITRs) and XhoI which cuts once in the AAV genome. These digests and the uncut construct were then run on a 1% Agarose gel and imaged on a ChemiDoc™. If the plasmid was >90% supercoiled, the correct size, and the ITRs were intact, the construct moved on to be tested via nucleofection and subsequently used for AAV vector production.

TABLE 23
Guide sequences cloned into p59.491.U6.X.Y. plasmids
(X = guide; Y = spacer)

| | | gRNA Guide | gRNA Guide + |
|---|---|---|---|
| Guide.spacer | Spacer Sequence | Sequence | Spacer Sequence |
| Construct | (SEQ ID NO) | (SEQ ID NO) | (SEQ ID NO) |
| 174.11.30 | 3627 | 3631 | 3643 |
| 229.11.30 | 3627 | 3632 | 3644 |
| 230.11.30 | 3627 | 3633 | 3645 |
| 231.11.30 | 3627 | 3634 | 3646 |
| 232.11.30 | 3627 | 3635 | 3647 |
| 233.11.30 | 3627 | 3636 | 3648 |
| 234.11.30 | 3627 | 3637 | 3649 |
| 235.11.30 | 3627 | 3638 | 3650 |
| 236.11.30 | 3627 | 3639 | 3651 |
| 237.11.30 | 3627 | 3640 | 3652 |
| 174.11.31 | 3628 | 3641 | 3653 |
| 235.11.31 | 3628 | 3631 | 3654 |
| 174.11.1 | 3629 | 3631 | 3655 |
| 235.11.1 | 3629 | 3642 | 3656 |
| 235.NT | 3630 | 3631 | 3657 |

TABLE 24
Guide sequences cloned into p59.491.U6.X.Y. plasmids. (X =
guide; Y = spacer) with spacer length variants

| | | Spacer | Guide | Guide + Spacer |
|---|---|---|---|---|
| Guide.spacer | Spacer | Sequence | Sequence | Sequence |
| Construct | length | (SEQ ID NO) | (SEQ ID NO) | (SEQ ID NO) |
| 174.11.30 | 20 nt | 3658 | 3669 | 3671 |
| 174.11.39 | 19 nt | 3659 | 3669 | 3672 |
| 174.11.38 | 18 nt | 3660 | 3669 | 3673 |
| 174.11.31 | 20 nt | 3661 | 3669 | 3674 |
| 174.11.37 | 19 nt | 3662 | 3669 | 3675 |
| 174.11.36 | 18 nt | 3663 | 3669 | 3676 |
| 235.11.1 | 20 nt | 3664 | 3670 | 3677 |
| 235.11.41 | 19 nt | 3665 | 3670 | 3678 |
| 235.11.40 | 18 nt | 3666 | 3670 | 3679 |
| 235.11.2 | 20 nt | 3667 | 3670 | 3680 |
| 235.11.43 | 19 nt | 3668 | 3670 | 3681 |
| 235.11.42 | 18 nt | 40542 | 3670 | 3682# |

TABLE 25
Sequences of AAV vector components common to the plasmids

| | | DNA sequence | Length of |
|---|---|---|---|
| Component | Part Name | or SEQ ID NO | Component (bp) |
| 5′ITR | | 3683 | 130 |
| buffer seq | | 3684 | 23 |
| enhancer | CMV | 3685 | 380 |
| Pol II promoter | CMV | 3619 | 204 |
| buffer seq | | 3686 | 15 |
| Kozak | | 3687 | 12 |
| start codon | MA | ATGGCC | 6 |
| 5′NLS | SV40 | 3689 | 21 |
| 5′linker | SR | TCTAGA | 6 |
| 3′NLS linker | GS | GGATCC | 6 |
| 3′NLS | SV40 | 3692 | 21 |
| tag | HA | 3693 | 27 |
| linker | GS | GGATCCTAA | 9 |
| buffer seq | | 3695 | 30 |
| Poly(A) | BgH poly A | 3696 | 209 |
| buffer seq | | GGTACCGT | 8 |
| Pol III promoter | U6 promoter | 3698 | 242 |
| buffer | | GAAACACC | 8 |
| buffer | | 3700 | 17 |
| 3′ITR | | 3701 | 141 |

TABLE 26
DNA sequences encoding CasX utilized in AAV

| CasX | SEQ | Length of coding |
|---|---|---|
| protein no. | ID NO: | sequence (bp) |
| 438 | 3702 | 2931 |
| 491 | 3703 | 2932 |
| 527 | 3704 | 2934 |
| 535 | 3705 | 2935 |
| 536 | 3706 | 2935 |
| 537 | 3707 | 2935 |
| 583 | 3708 | 2935 |
| 668 | 3709 | 2938 |
| 672 | 3710 | 2934 |
| 669 | 3711 | 2938 |
| 670 | 3712 | 2938 |
| 676 | 3713 | 2938 |

### Reporter Cell Lines:

A neural progenitor cell line isolated from the Ai9-tdTomato was cultured in suspension in pre-equilibrated mNPC medium (DMEM/F12 with GlutaMax™, 10 mM HEPES, 1×MEM Non-Essential Amino Acids, 1× penicillin/streptomycin, 1:1000 2-mercaptoethanol, 1×B-27 supplement, minus vitamin A, 1×N2 with supplemented growth factors bFGF and EGF). Prior to testing, cells were dissociated using accutase, with gentle resuspension, monitoring for complete separation of the neurospheres. Cells were then quenched with media, spun down and resuspended in fresh media. Cells were counted and directly used for nucleofection or 10,000 cells were plated in a 96-well plate coated with PLF (1×Poly-DL-ornithine hydrobromide, 10 mg/mL in sterile diH20, 1× Laminin, and 1× Fibronectin), 2 days prior to AAV transduction.

A HEK293T dual reporter cell line was generated by knocking into HEK293T cells two transgene cassettes that constitutively expressed exon 1 of the human RHO gene linked to GFP and exon 1 of the human P23H.RHO gene linked to mScarlet. The modified cells were expanded by serial passage every 3-5 days and maintained in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× ThermoFisher #11140050), HEPES buffer (100× ThermoFisher #15630080), and 2-mercaptoethanol (1000× ThermoFisher #21985023). The cells were incubated at 37° C. and 5% CO2. After 1-2 weeks, GFP+/mscarlet+ cells were bulk sorted into FB medium. The reporter lines were expanded by serial passage every 3-5 days and maintained in FB medium in an incubator at 37° C. and 5% CO2. Reporter clones were generated by a limiting dilution method. The clonal lines were characterized via flow cytometry, genomic sequencing, and functional modification of the RHO locus using a previously validated RHO targeting CasX molecule. The optimal reporter lines were identified as ones that: i) had a single copy of WTRHO.GFP and mutRHO.mScarlet correctly integrated per cell; ii) maintained doubling times equivalent to unmodified cells; and iii) resulted in reduction in GFP and mscarlet fluorescence upon disruption of the RHO gene when assayed using the methods described below.

### Plasmid Nucleofection:

AAV cis-plasmids driving expression of the CasX-scaffold-guide system were nucleofected in mNPCs using the Lonza P3 Primary Cell 96-well Nucleofector Kit. For the ARPE-19 line, the Lonza SF solution and supplement was used. Plasmids were diluted to concentrations of 200 ng/μl, 100 ng/μL. 5 μL of DNA per construct was added to the P3 or SF solution containing 200,000 tdTomato mNPCs or ARPE-19 cells respectively. The combined solution was nucleofected using a Lonza 4D Nucleofector System according to manufacturer's guidelines. Following nucleofection, the solution was quenched with appropriate culture media. The solution was then aliquoted in triplicate (approx. 67,000 cells per well) in a 96-well plate. 48 hours after transfection, treated mNPCs were replenished with fresh mNPC media containing growth factors and treated ARPE-19 cells were replenished with fresh FB medium. 5 days after transfection, tdTomato mNPCs and ARPE-19 cells were lifted and activity was assessed by FACS.

### AAV Vectors Production:

Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free Opti-MEM™ media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant) clarified further using a 0.45 μM filter.

To determine the viral genome titer, 1 μL from crude lysate viruses was digested with DNase and proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region or a 62 bp-fragment located in the AAV2-ITR. Ten-fold serial dilutions (5 pl each of 2e+9 to 2e+4 DNA copies/mL) of an AAV ITR plasmid was used as reference standards to calculate the titer (viral genome (vg)/mL) of viral samples.

### AAV Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Calculations were based on an estimated number of 20,000 cells per well at the time of transfection. Final volume of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

Assessing editing activity by FACS: 5 days after transfection, treated tdTomato mNPCs or ARPE-19 cells in 96-well plates were washed with dPBS and treated with 50 μL TrypLE and Trypsin (0.25%) for 15 and 5 minutes respectively. Following cell dissociation, treated wells were quenched with media containing DMEM, 10% FBS and 1×penicillin/streptomycin. Resuspended cells were transferred to round-bottom 96-well plates and centrifuged for 5 min at 1000×g. Cell pellets were then resuspended with dPBS containing 1×DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

NGS analysis of indels at mRHO exon 1 locus: 5 days after transfection, treated tdTomato mNPCs in 96-well plates were washed with dPBS and treated with 50 μL TrypLE and trypsin (0.25%) for 15 and 5 minutes respectively. Following cell dissociation, treated wells were quenched with media containing DMEM, 10% FBS and 1× penicillin/streptomycin. Cells were then spun down and resulting cell pellets washed with PBS prior to processing them for gDNA extraction using the Zymo mini DNA kit according to the manufacturer's instructions. For assessing editing levels occurring at the mouse RHO exon 1 locus, amplicons were amplified from 200ng of gDNA with a set of primers targeting the RHO exon 1 locus, bead-purified (Beckman coulter, Agencourt Ampure XP) and then re-amplified to incorporate Illumina™ adapter sequences. Specifically, these primers contained an additional sequence at the 5′ ends to introduce Illumina™ read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq™ according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Results

Different editing experiments were conducted to quantify on-target cleavage mediated by CasX 491 paired with gRNA scaffold variants (guides 174 & 229-237) with different spacers targeting multiple genomic loci of interest. Constructs were cloned into the AAV backbone p59, flanked by ITR2 sequences, driving expression of the protein Cas 491 under the control of a CMV promoter, as well as the scaffold-spacer under the control of the human U6 promoter.

The mNPC-tdT reporter cell line was used to assess single-cut efficiency at the endogenous mouse RHO exon 1 locus (spacer 11.30, CTC PAM). A dual reporter system integrated in a ARPE-19 derived cell line was also used to assess on-target editing at the exogenously expressed human WT RHO locus (spacer 11.1, CTC PAM).

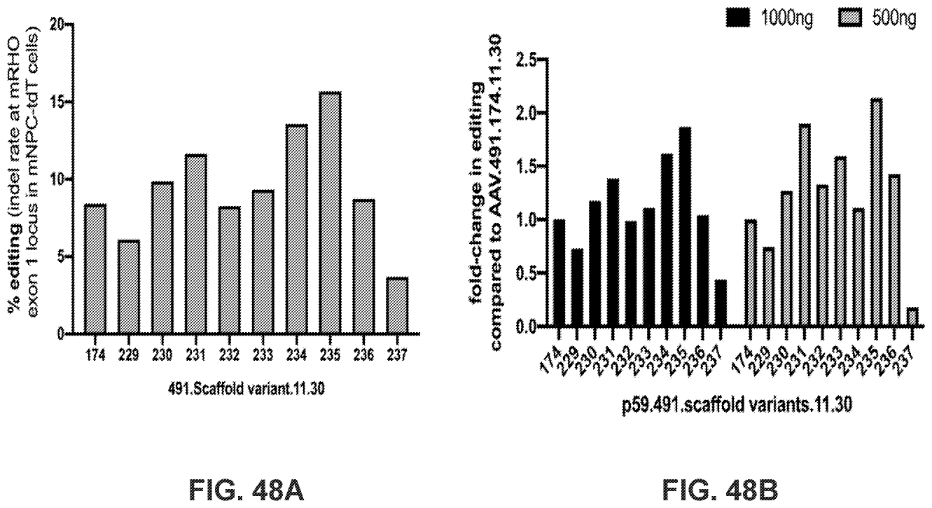

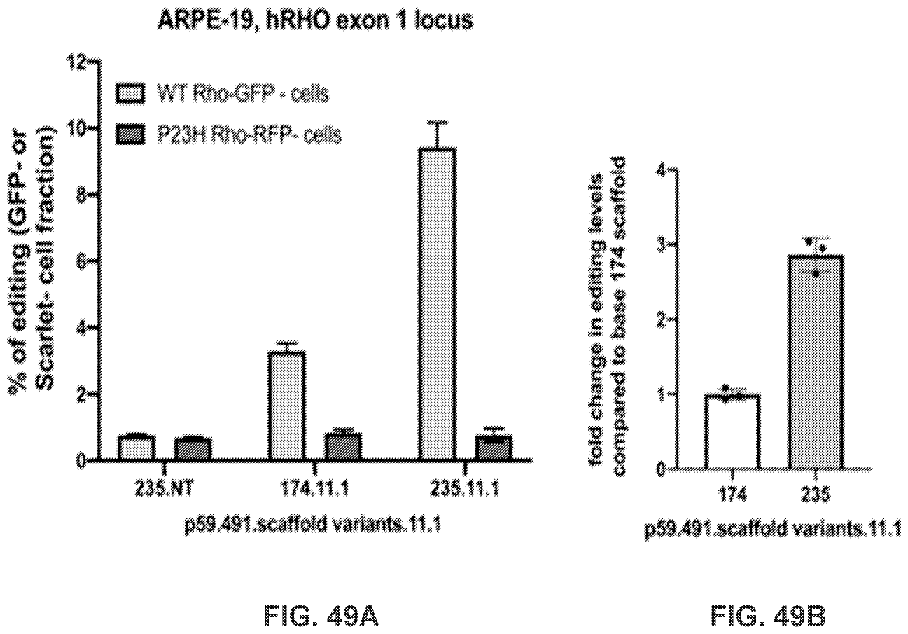

Scaffold variants with spacer 11.30 were tested via nucleofection in the mouse NPC cell line at two different doses, 1000ng and 500ng. Constructs were compared to the current benchmark gRNA scaffold 174 activity. Constructs expressing scaffold variants 231, 233, 234, 235 performed at higher levels than ones with scaffold 174.11.30 (FIGS. 48A and 48B). Scaffold 235 displayed a 2-fold increased activity at mRHO exon 1 locus compared to gRNA scaffold 174. Whether scaffold 235 consistently improved activity without increased off-target cleavage was further validated by nucleofecting the dual reporter ARPE-19 cell line with construct p59.491.174.11.1 and p59.491.235.11.1, as well as a non-target spacer control. Spacer 11.1 was targeting the exogenously expressed hRHO-GFP gene. Scaffold 235 displayed 3-fold increased activity compared to 174 (9% vs 3% of Rho-GFP− cells respectively, FIGS. 49A and 49B). Allele-specificity was assessed by looking at the frequency of hP23H-RHO-Scarlet-cell population, whose sequence differs from the wild-type by 1 bp.

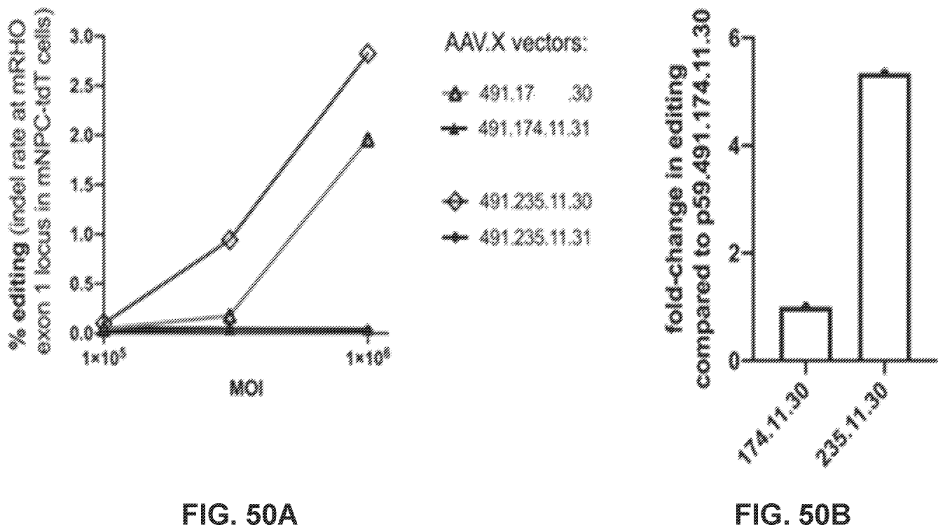

Experiments were performed to test whether these scaffold variants packaged efficiently in AAV and remained potent when delivered virally. mNPC transduced with AAV vectors expressing guide scaffold 235 with spacer 11.30 (on-target, mouse WT RHO) showed increased activity at the on-target locus (>5-fold increase, FIGS. 50A and 59B) compared to ones infected with AAV.491.174.11.30 at 3.0e+5 MOI, with significant no off-target indels detectable with both AAV.491.174.11.31 and AAV.491.235.11.31 vectors targeting the P23H-RHO SNP, respectively.

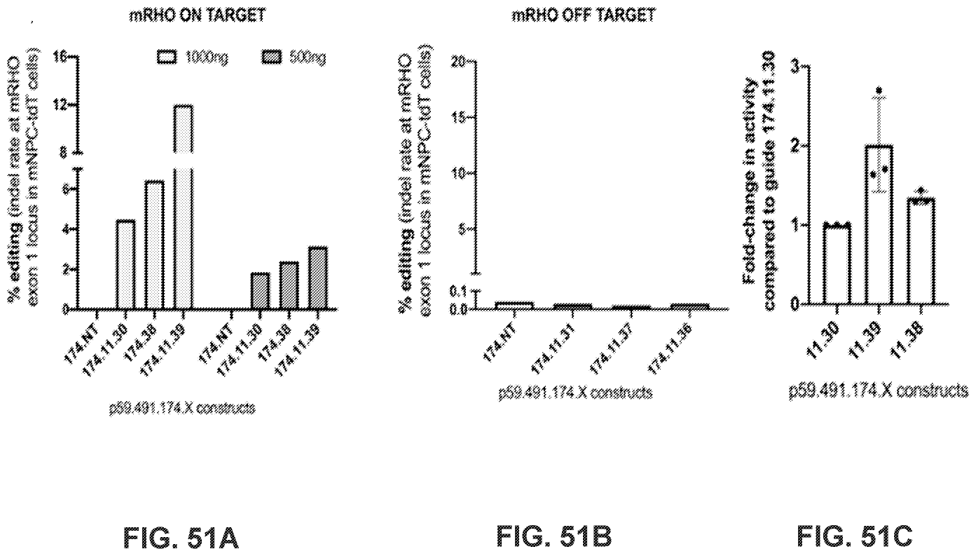

Assessing effects of spacer length: Another set of experiments was conducted to test whether spacer length variants could improve on-target activity. Spacers 11.39, 11.38 and spacer 11.37 (19 nt P23H RHO), 11.36 (18 nt P23H RHO) were designed from parental spacer 11.30 (20 nt WT RHO) and 11.31 (20 nt P23H RHO), respectively, harboring 1 or 2 bp truncations on the 3′ end of the sequence. mfNPC-tdT cells were nucleofected with 1000 ng and 500 ng of constructs p59.491.174.11.30 (20 nt WT RHO), p59.491.174.11.39 (19 nt WT RHO), p49.491.174.11.38 (18 nt WT RHO), and editing levels were assessed 5 days later. All truncated spacer versions improved editing levels (FIGS. 51A and 51C), with highest improvement observed with p59.491.11.39 constructs (˜2-fold improvement achieved with the 19 bp spacer relative to the 20 bp spacer length construct). No increase in off-target cleavage was observed with truncation spacer variants of the 11.31 spacer targeting the mouse P23H-RHO locus (FIG. 51B).

These results support that scaffold variants with structural mutations can be engineered with increased activity in dual reporter systems investigating therapeutically relevant genomic targets such as the mouse and human RHO exon 1 loci. Furthermore, while the newly characterized scaffold displayed overall >2-fold increase in activity, no off-target cleavage with a 1-bp mismatch spacer region was detected. This is relevant for allele-specific therapeutic strategy such as autosomal dominant retinitis pigmentosa P23H RHO, which mutated allele differs from WT sequence by 1 nucleotide, targeted by spacer 11.31. This study further validates the use of guide scaffold 235 in AAV vectors designed for P23H RHO rescue and genotoxic studies as well as for other therapeutic targets.

### Example 13: Improved Scaffold and Guide Variants Demonstrate Enhanced On-Target Activity In Vivo

Experiments were conducted to demonstrate that engineered CasX & gRNA-guide and spacer variants harboring structural mutations that improve selectivity and on-target activity lead to increase edits when delivered in vivo to photoreceptors in the mouse retina, with a spacer targeting the P23 residue at a therapeutically relevant level in the WT. Here, it was assessed whether vector expressing CasX variant 491, guide variant 235 and spacer 11.39 improves editing levels compared to parental CasX 491, guide variant 174 and spacer 11.30 in vivo.

### Materials and Methods

Generation of AAV Plasmids and Viral Vectors: The CasX variant 491 under the control of the RHO promoter, and gRNA.guide variant 174 with spacer 11.30 and spacer 11.31 (AAGTGGCTCCGCACCACGCC (SEQ ID NO: 3628)) or gRNA-guide variant 235 with spacer 11.39 (AAGGGGCTCCGCACCACGCC (SEQ ID NO: 3658)) and 11.37 (AAGTGGCTCCGCACCACGC (SEQ ID NO: 3662)) targeting mouse RHO exon 1 at P23 residues) under the U6 promoter were cloned into the p59 plasmid flanked with AAV2 ITR.

Cloning: Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. Cas X variant 491 under the RHO promoter and scaffold variants 174 and 235, under the control of the human U6 promoter, were cloned into an AAV backbone, flanked by AAV2 ITRs. Spacers 11.30, 11.31 and variants 11.39, 11.37 were cloned respectively into pAAV.RHO.491.174 and pAAV.RHO.491.235 using Golden Gate cloning. New AAV constructs were then transformed into chemically competent E. coli (Stbl3s). Validated constructs were maxi-prepped. To assess the quality of maxi-preps, constructs were processed in two separate digests with XmaI (which cuts at several sites in each of the ITRs) and XhoI which cuts once in the AAV genome. If the plasmid was >90% supercoiled, the correct size, and the ITRs were intact, the construct was subsequently used for AAV vector production.

### AAV Vectors Production:

Suspension HEK293T cells were adapted from parental HEK293T and grown in FreeStyle 293 media. 500 mL cultures were diluted to a density of 2e+6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free Opti-MEM™ media. Three days later, cultures were centrifuged to separate the supernatant from the cell pellet, and the AAV particles were collected, concentrated, and filtered following standard procedures.

To determine the viral genome titer, 1 μL from crude lysate viruses was digested with DNase and proteinase K, followed by quantitative PCR. 5 μL of digested virus was used in a 25 μL qPCR reaction composed of IDT primetime master mix and a set of primers and 6′FAM/Zen/IBFQ probe (IDT) designed to amplify a 62 bp-fragment located in the AAV2-ITR. An AAV ITR plasmid was used as reference standards to calculate the titer (viral genome (vg)/mL) of viral samples.

Subretinal injections C57BL6J mice were obtained from the Jackson Laboratories and were maintained in a normal 12-hour light/dark cycle. Subretinal injections were performed on 3-4 weeks old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with Genteal® gel during the surgery. Under a surgical microscope, an ultrafine 30½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each mouse from the experimental groups was injected with 1.5.0e+9 viral genome (vg)/eye.

NGS analysis: 3 weeks post-injection, animals were sacrificed, and the eyes enucleated in fresh PBS. Whole retinae were isolated from the eye cups and processed for gDNA extraction using the DNeasy® Blood & Tissue Kit (Qiagen) according to the manufacturer's instructions. Amplicons were amplified from 200 ng of gDNA with a set of primers targeting the mouse RHO, exon 1 locus, bead-purified (Beckman coulter, Agencourt Ampure XP) and then re-amplified to incorporate Illumina™ adapter sequences. Specifically, these primers contained an additional sequence at the 5′ ends to introduce Illumina™ read and 2 sequences, as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq™ according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Results

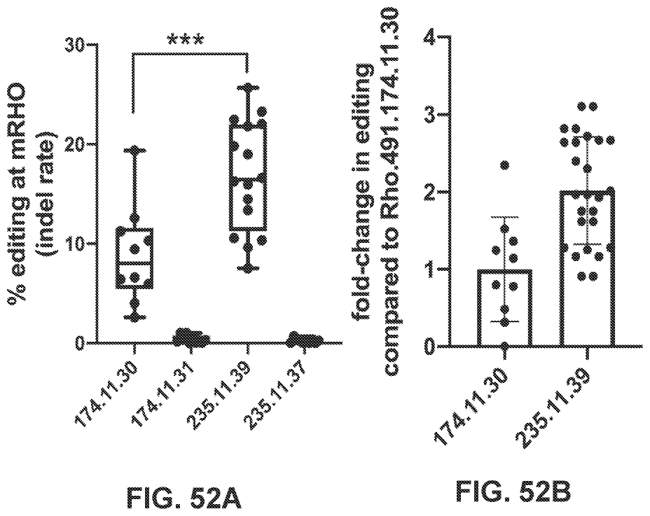

The benchmark vector, AAV.491.174.11.30 (on-target) achieved ˜8% editing across all samples (FIG. 52A; n=8 retinas). A similar vector with spacer 11.31 (off-target, 1 bp mismatch from 11.30 targeting P23H-RHO SNP) showed background level of editing (˜0.4%). An AAV vector expressing scaffold variant 235 and spacer 11.39 achieved over a 2-fold improvement relative to the AAV.491.174.11.30 parental vector (FIG. 52B), with a mean of 16% editing, and as high as 25% in some retinas. This increase in on-target editing remained selective, as no increase in off-target with spacer 11.37 (targeting P23H-RHO SNP, 1 bp-mismatch compared to spacer 11.39) levels compared to AAV.491.174.11.31 parental vector.

These experiments demonstrate proof-of-concept that CasX 491 expression driven by a rod photoreceptor-selective promoter with scaffold 174, and a spacer targeting the mouse P23 RHO locus can achieve therapeutic-relevant levels of edits at the P23 mouse locus when sub-retinally delivered via AAV in the murine retina. These results also support that editing levels achieved from engineered gRNA guide (235) and spacer variants (11.39) screened previously in vitro translate as well in vivo, and retain allele-specific selectivity. This study further validates the use of guide scaffold 235 in AAV vectors designed for P23H RHO rescue and genotoxic studies, as well as for other therapeutic targets.

The results of Examples 11 and 12 support that scaffold variants with structural mutation can be engineered with increased activity in dual reporter systems investigating therapeutically relevant genomic targets such as the mouse and human RHO exon 1 loci. Furthermore, while the newly characterized 235 scaffold displayed an overall >2-fold increase in activity, no off-target cleavage with 1-bp mismatch spacer region was detected. This is relevant for allele-specific therapeutic strategy such as autosomal dominant retinitis pigmentosa P23H RHO, which mutated allele differs from WT sequence by 1 nucleotide, targeted by spacer 11.31. The present study was conducted to further validate the use of guide scaffold 235 in AAV vectors designed for mouse P23H RHO rescue and genotoxic studies, as well as for other therapeutic targets.

### Example 14: Improved CasX Variants Demonstrate Enhanced On-Target Activity In Vitro

The CasX protospacer adjacent motif allows for genomic targeting with precision, which is necessary for various genome editing therapeutic applications, such as autosomal dominant RHO, which requires an allele-specific targeting of the P23H mutation without altering the wild-type sequence.

Experiments were conducted to investigate whether rationally-designed engineered CasX nucleases, with introduced mutations predicted to increase CTC-PAM mediated on-target activity while keeping fidelity high, and with reduced off-target events, improved editing levels at the endogenous mouse RHO locus when delivered in vivo to rod photoreceptors cells,

Additionally, experiments were conducted to further validate the use of guide scaffold 235 in AAV vectors designed for mouse P23H RHO rescue and genotoxic studies, as well as for other therapeutic targets.

### Methods

CasX protein variants identified in different assays looking at PAM activity were selected for their increased activity at CTC PAM. The CasX proteins were cloned into an AAV transgene construct for plasmid and viral vector validation. The AAV transgene was conceptually broken up between ITRs into different parts, which consisted of the therapeutic cargo and accessory elements relevant to expression in mammalian cells and the nuclease-guide RNA complex (Protein, scaffold, spacer).

### Cloning:

Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. New AAV constructs were then transformed into chemically competent E. coli (Stbl3s). Validated constructs were maxi-prepped. To assess the quality of maxi-preps, constructs were processed in two separate digests with XmaI (which cuts at several sites in each of the ITRs) and XhoI which cuts once in the AAV genome. These digests and the uncut construct were then run on a 1% agarose gel. If the plasmid was >90% supercoiled, the correct size, and the ITRs were intact, the construct moved on to be tested via nucleofection and subsequently used for AAV vector production.

### Reporter Cell Lines:

An immortalized neural progenitor cell line isolated from the Ai9-tdTomato was cultured in suspension in pre-equilibrated mNPC medium (DMEM/F12 with GlutaMax™, 10 mM HEPES, 1×MEM Non-Essential Amino Acids, 1× penicillin/streptomycin, 1:1000 2-mercaptoethanol, 1×B-27 supplement, minus vitamin A, 1×N2 with supplemented growth factors bFGF and EGF. Prior to testing, cells were lifted using accutase, with gentle resuspension, monitoring for complete separation of the neurospheres. Cells were then quenched with media, spun down and resuspended in fresh media. Cells were counted and directly used for nucleofection or 10,000 cells were plated in a 96-well plate coated with PLF (1×Poly-DL-ornithine hydrobromide, 10 mg/mL in sterile diH20, 1×Laminin, and 1×Fibronectin), 2 days prior to AAV transduction.

A HEK293T dual reporter cell line was generated by knocking into HEK293T cells two transgene cassettes that constitutively expressed exon 1 of the human RHO gene linked to GFP and exon 1 of the human P23H.RHO gene linked to mscarlet. The modified cells were expanded by serial passage every 3-5 days and maintained in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× ThermoFisher #11140050), HEPES buffer (100× ThermoFisher #15630080), and 2-mercaptoethanol (1000× ThermoFisher #21985023). The cells were incubated at 37° C. and 5% CO2. After 1-2 weeks, GFP+/mscarlet+ cells were bulk sorted into FB medium. The reporter lines were expanded by serial passage every 3-5 days and maintained in FB medium in an incubator at 37° C. and 5% CO2. Reporter clones were generated by a limiting dilution method. The clonal lines were characterized via flow cytometry, genomic sequencing, and functional modification of the RHO locus using a previously validated RHO targeting CasX molecule. The optimal reporter lines were identified as ones that: i) had a single copy of WT-RHO.GFP and P23H-RHO.mScarlet correctly integrated per cell; ii) maintained doubling times equivalent to unmodified cells; and iii) resulted in reduction in GFP and mScarlet fluorescence upon disruption of the RHO gene when assayed using the methods described below.

### Plasmid Nucleofection:

AAV cis-plasmids driving expression of the CasX-scaffold-guide system were nucleofected in mNPCs using the Lonza P3 Primary Cell 96-well Nucleofector Kit. For the ARPE-19 line, the Lonza SF solution and supplement was used. Plasmids were diluted to concentrations of 200 ng/ul, 100 ng/μL. 5 μL of DNA per construct was added to the P3 or SF solution containing 200,000 tdTomato mNPCs or ARPE-19 cells respectively. The combined solution was nucleofected using a Lonza 4D Nucleofector System according to manufacturer's guidelines. Following nucleofection, the solution was quenched with appropriate culture media. The solution was then aliquoted in triplicate (approx. 67,000 cells per well) in a 96-well plate. 48 hours after transfection, treated cells were replenished with fresh mNPC media containing growth factors. 5 days after transfection, tdTomato mNPCs were lifted and activity was assessed by FACS.

### AAV Vectors Production:

Suspension HEK293T cells were adapted from parental HEK293T and grown in FreeStyle 293 media. For screening purposes, small scale cultures (20-30 mL) were diluted to a density of 1.5e+6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free Opti-MEM™ media. Three days later, cultures were centrifuged to separate the supernatant from the cell pellet, and the AAV particles were collected, concentrated, and filtered following standard procedures.

To determine the viral genome titer, 1 μL from crude lysate viruses was digested with DNase and proteinase K, followed by quantitative PCR. 5 μL of digested virus was used in a 25 μL qPCR reaction composed of IDT primetime master mix and a set of primers and 6′FAM/Zen/IBFQ probe (IDT) designed to amplify the CMV promoter region or a 62 nucleotide-fragment located in the AAV2-ITR. Ten-fold serial dilutions (5 pl each of 2e+9 to 2e+4 DNA copies/mL) of an AAV ITR plasmid was used as reference standards to calculate the titer (viral genome (vg)/mL) of viral samples.

### AAV Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Calculations were based on an estimated number of 20,000 cells per well at the time of transfection. Final volumes of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

### Assessing Editing Activity by FACS:

5 days after transfection, treated tdTomato mNPCs or ARPE-19 cells in 96-well plates were washed with dPBS and treated with 50 μL TrypLE and Trypsin (0.25%) for 15 and 5 minutes, respectively. Following cell dissociation, treated wells were quenched with media containing DMEM, 10% FBS and 1× penicillin/streptomycin. Resuspended cells were transferred to round-bottom 96-well plates and centrifuged for 5 min at 1000×g. Cell pellets were then resuspended with dPBS containing 1×DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

NGS Analysis of Indels at mRHO Exon I Locus:

5 days after transfection, treated tdTomato mNPCs in 96-well plates were washed with dPBS and treated with 50 μL TrypLE and trypsin (0.25%) for 15 and 5 minutes, respectively. Following cell dissociation, treated wells were quenched with media containing DMEM, 10% FBS and 1× penicillin/streptomycin. Cells were then spun down and resulting cell pellets washed with PBS prior to processing them for gDNA extraction using the Zymo mini DNA kit according to the manufacturer's instructions. For assessing editing levels occurring at the mouse RHO exon 1 locus, amplicons were amplified from 200 ng of gDNA with a set of primers targeting the mouse RHO exon 1 locus, bead-purified (Beckman coulter, Agencourt Ampure XP) and then re-amplified to incorporate Illumina™ adapter sequences. Specifically, these primers contained an additional sequence at the 5′ ends to introduce Illumina™ read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Results

Engineered mutations in prior assays identified CasX variants with the ability to increase both overall activity, specificity of the nuclease, as well as increased activity with spacers targeting CTC-PAM sites. These mutations to the CasX 491 protein gave rise to CasX variant proteins 515, 527, 528, 535, 536 and 537 (see Table 5 for sequences).

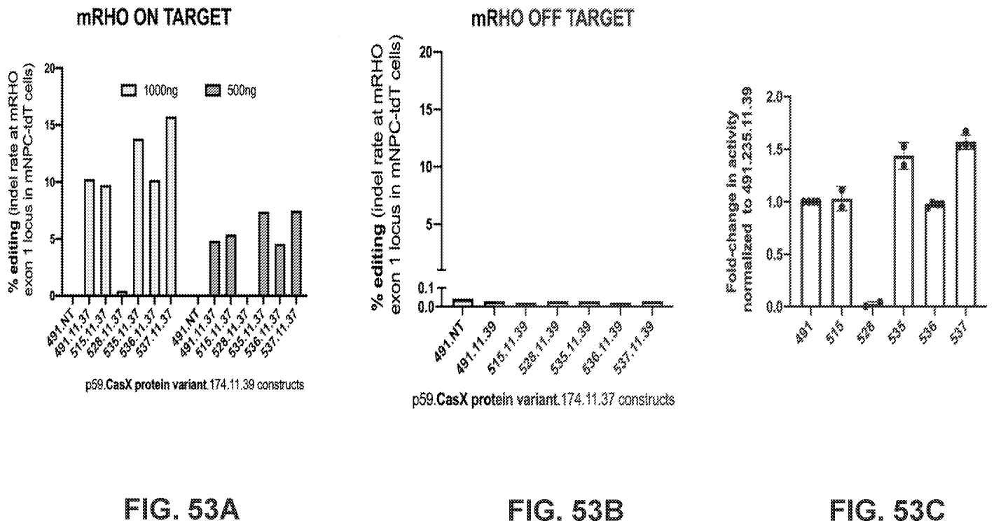

Multiple editing screens were conducted to quantify on-target editing levels mediated by these CasX variant proteins paired with gRNA scaffolds 174 or 235 and different spacers targeting multiple genomic loci of interest (the encoding sequences of the guides and spacers are presented in Tables 23 and 24). Constructs were cloned into the AAV backbone p59, flanked by ITR2 sequences, driving expression of the Cas X under the control of a CMV promoter, as well the scaffold-spacer under the control of the human U6 promoter. The mNPC-tdT reporter cell line was used to assess single-cut efficiency at the endogenous mouse RHO exon 1 locus (spacer 11.39, CTC PAM, FIG. 53A). A dual reporter system integrated in an ARPE-19 derived cell line was also used to assess on-target editing at the exogenously expressed human WT RHO locus (spacer 11.41, CTC PAM) or at the P23H-RHO locus (spacer 11.43, CTC PAM, FIG. 53B).

The CasX protein variants with spacer 11.39 were tested via nucleofection in the mouse NPC cell line at two different doses, 1000 ng and 500 ng. Constructs were compared to the parental CasX 491 activity. AAV constructs expressing CasX 535 and 537 with scaffold 174 and spacer 11.30 demonstrated the greatest editing activity at the mRHO exon 1 locus of any of the CasX variants (by percent editing, FIG. 53A), which was increased 1.5-fold relative to CasX 491 (FIG. 53C, normalized to 1), without increased off-target cleavage, shown by the nucleofection of the protein variants with spacer 11.37 (targeting mutant P23H-RHO allele, FIG. 53B).

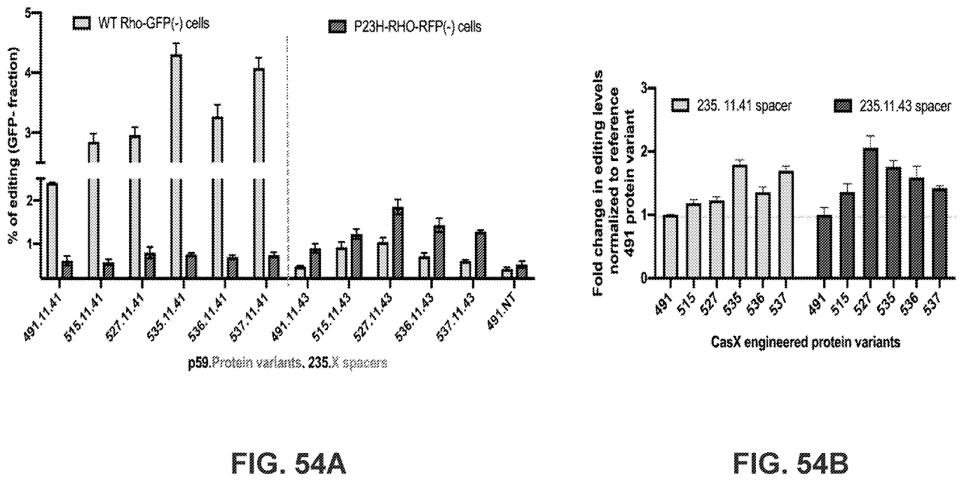

Experiments were then conducted to determine whether the improvements observed at the mouse RHO locus with the mutated variants translated at the human RHO locus, which is more clinically-relevant. The dual reporter ARPE-19 cell line was nucleofected with constructs expressing the CasX variant proteins paired with either gRNA-scaffold 235 with spacer 11.41 or spacer11.43, targeting human RHO. CasX 535 and 537 also displayed over 1.5-fold increased editing activity compared to CasX 491 (˜4.3% and 4.1% editing compared to 2.4% editing of Rho-GFP− cells respectively, FIGS. 54A and 54B) when targeting the exogenous WT-RHO-GFP locus. Constructs expressing CasX variants 515, 527 and 536 edited at similar levels to CasX 491. Interestingly, when using a spacer targeting the P23H-RHO-mScarlet locus, all the variant proteins demonstrated improved editing compared to CasX 491. The highest activity levels were achieved by constructs expressing CasX 527 (2-fold increase) and CasX 535 (1.8-fold increase).

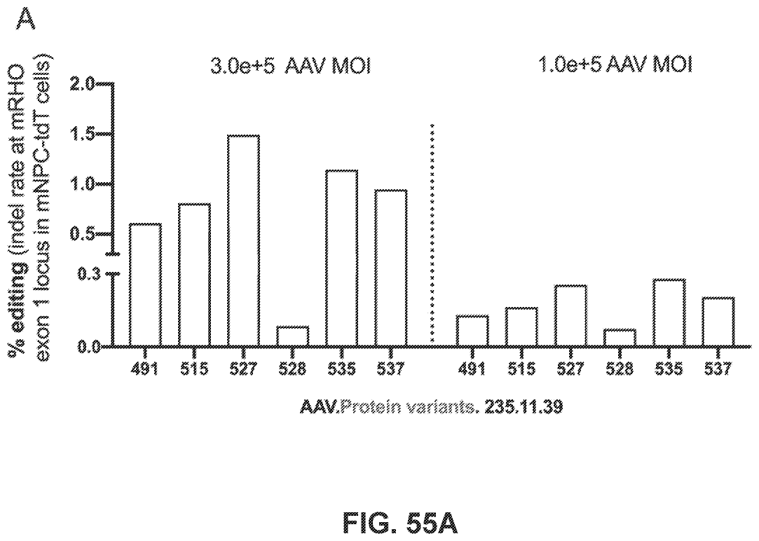

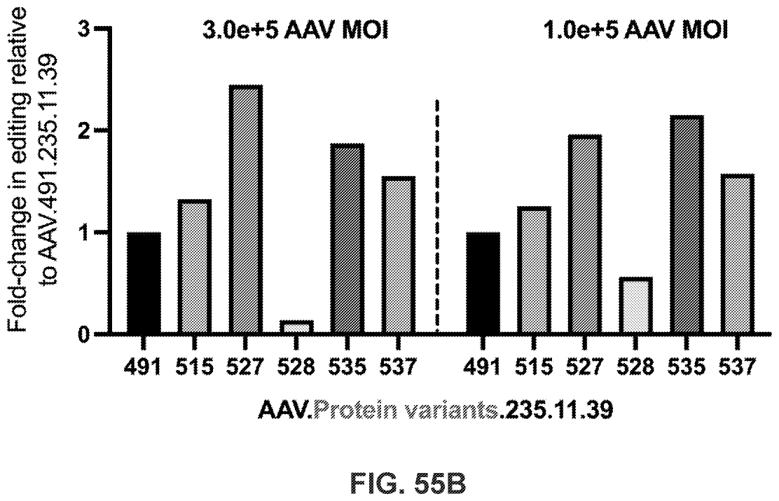

Finally, experiments were performed to assess whether these protein variants packaged efficiently in AAV and remained potent when delivered virally. mNPC transduced with AAV vectors expressing CasX 527, 535 and 537 and guide scaffold 235 with spacer 11.39 (on target, mouse WT RHO) showed increased activity at the on-target locus (>2-fold increase, FIGS. 55A and 55B) relative to AAV CasX 491 and guide scaffold 235 with spacer 11.39 with transduction at 3.0e+5 MOI. Fold-improvement in activity were observed in a dose-dependent manner.

These results support that CasX variants with structural mutations can be engineered resulting in increased editing activity in dual reporter systems at therapeutically-relevant genomic targets, such as the mouse and human RHO exon 1 loci. Furthermore, while the newly-characterized variants displayed an overall 1.5-2-fold increase in activity, they retained allele-specific targeting with no off-target cleavage detected with a 1-bp mismatch spacer. This is relevant for allele-specific therapeutic strategy, such as editing at autosomal dominant retinitis pigmentosa P23H RHO, where the mutated allele differs from WT sequence by 1 nucleotide (targeted by spacer 11.37). This study further validates the use of CasX variants 527, 535, 536 with scaffold 235 in AAV vectors designed for P23H RHO rescue and genotoxic studies, as well as for other therapeutic targets.

### Example 15: AAV Constructs with CasX and Targeted Guides Edit the P23 RHO Locus In Vivo in C57BL/6J Mice

Experiments were conducted to demonstrate the ability of CasX to edit in vivo the endogenous RHO locus in the mouse retina, with a spacer targeting the P23 residue at a therapeutically relevant level, to generate proof-of-concept data that is expected to justify and inform experiments in the P23H mouse disease model. Here, it was assessed whether CasX variant 491 and guide variant 174, and a spacer targeting the P23 locus of the mouse RHO gene can generate significant, detectable in the retina when injected sub-retinally, and evaluate efficacy and safety of two different viral doses (1.0e+9 and 1.0e+10 vg). Rescue of 10% of rod photoreceptors can restore vision in cases of autosomal dominant retinitis pigmentosa (adRP). Therefore, editing 10% of the RHO loci in rod photoreceptors in the retina may provide a therapeutic benefit in a disease context by reducing the levels of the mutant rhodopsin protein and preventing rod photoreceptor degeneration.

### Materials and Methods

### Generation of AAV Plasmids and Viral Vectors:

The CasX variant 491 under the control of the CMV promoter and RNA guide variant 174/spacer 11.30 (AAGGGGCTCCGCACCACGCC (SEQ ID NO: 3627), targeting mouse RHO exon 1 at P23 residues) under the U6 promoter were cloned into a pAAV plasmid flanked with AAV2 ITR. AAV.491.174.11.30 vectors were produced in HEK293 cells using the triple-transfection method.

### Subretinal Injections:

C57BL/6J mice were obtained from the Jackson Laboratories and maintained in a normal 12-hour light/dark cycle. Subretinal injections were performed on 5-6 weeks old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with Genteal® gel during the surgery. Under a surgical microscope, an ultrafine 30½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each experimental group (n=5) were injected in one eye with 1e+9 vg or 1e+10 viral genome (vg)/eye, and the contralateral eye injected with the AAV formulation buffer.

### NGS Analysis:

3 weeks post-injection, animals were sacrificed, and the eyes enucleated in fresh PBS. Whole retinae were isolated from the eye cups and processed for gDNA extraction using the DNeasy® Blood & Tissue Kit (Qiagen) according to the manufacturer's instructions. Amplicons were amplified from 200ng of gDNA with a set of primers targeting the mouse RHO, exon 1 locus, bead-purified (Beckman coulter, Agencourt Ampure XP) and then re-amplified to incorporate Illumina™ adapter sequences. Specifically, these primers contained an additional sequence at the 5′ ends to introduce Illumina™ read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq™ according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Immunohistology:

Mice were euthanized 3-4 weeks post-injection. Enucleated eyes were placed in 10% formalin overnight at 4° C. Retinae were dissected out from the eye cups, rinsed in PBS thoroughly and immersed in 15%-30% sucrose gradient. Tissues were embedded in optimal cutting temperature (OCT), froze on dry ice before being transferred to −80′C storage. 20 M sections were cut using a cryostat. The sections were blocked for ≥1 hour at room temperature in blocking buffer (2% normal goat serum, 1% BSA, 0.1% Triton-X 100) before antibody labeling. The antibodies used were anti-mouse HA (Abcam, 1:500) and Alexa Fluor 488 rabbit anti-mouse (Invitrogen, 1:2000). Sections were counterstained with DAPI to label nuclei, mounted on slides and imaged on a fluorescent microscope.

### Results

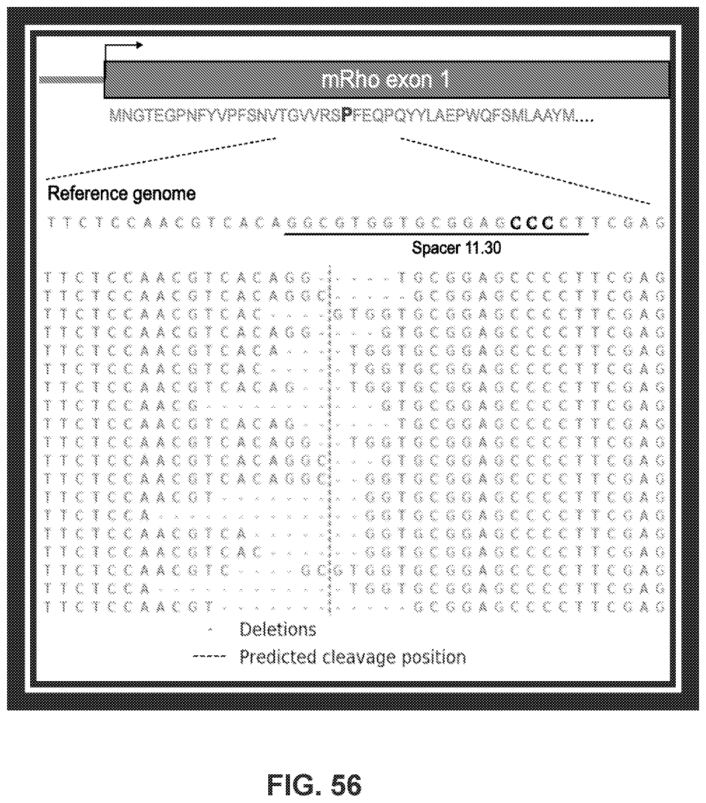

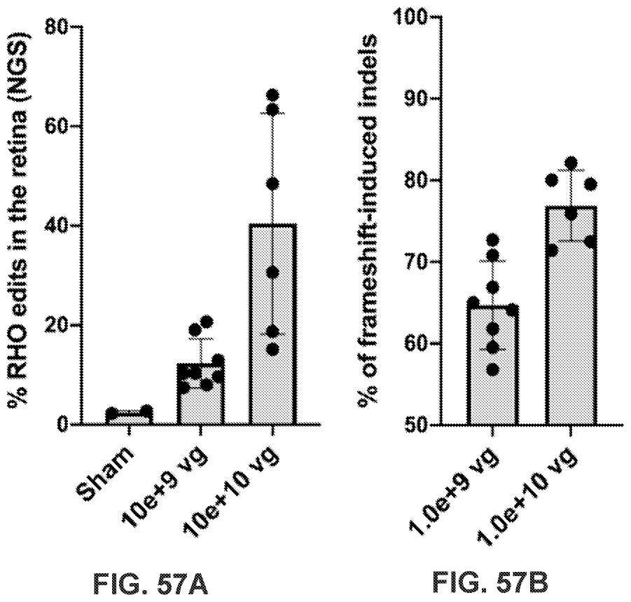

The ability of CasX to edit the P23 RHO locus in the mouse retina was assessed. Two therapeutically relevant doses, 1.0e+9 and 1.0E+10 vg of AAV-CasX.491.174.11.30 were administered in the subretinal space of 5-6 weeks old C57BL/6J mice. Three weeks post-injections, retinae were harvested and editing levels quantified via NGS and the CRISPResso analysis pipeline. The spacer 11.30 targets the WT P23 genomic locus (FIG. 56) located at the beginning of the first exon of RHO. Overexpression of CasX-491.174.11.30 led to significant, dose-dependent, editing of mRHO exon 1 locus in treated-compared to sham-injected retinae (FIGS. 57A-57B). The left panel (FIG. 57A) shows the quantification in % of total indels detected by NGS at the mouse P23 RHO locus in AAV-CasX or sham-injected retinae compared to the mouse reference genome. The right panel (FIG. 57B) shows the fraction (%) of edits predicted to lead to frameshift mutations in RHO protein. Data are presented as average of NGS readouts of editing outcomes from the entire retina, from six to eight animals per experimental cohort. The highest AAV dose, 1e+10 vg/eye, increased indels rate by 4-fold compared to the 1.0e+9 vg dose, with 40.3±22% versus 12.3±5% RHO editing detected respectively. The majority of indels generated by CasX.491 were deletions (left panel), predicted to translate to a high frequency of frameshift-mutations (64.7 versus 76.9% for 1.0e+9 and 1.0e+10 vg/dose respectively), and hypothetically high levels of RHO protein knock down. These results suggest that with a spacer driving allele-specific target of mutant P23H locus in the P23H+/− mouse model, CasX could efficiently editing 10% of rod photoreceptor, with the majority of edits translating to a knocking-down the mutant P23H RHO and significantly delay photoreceptor degeneration.

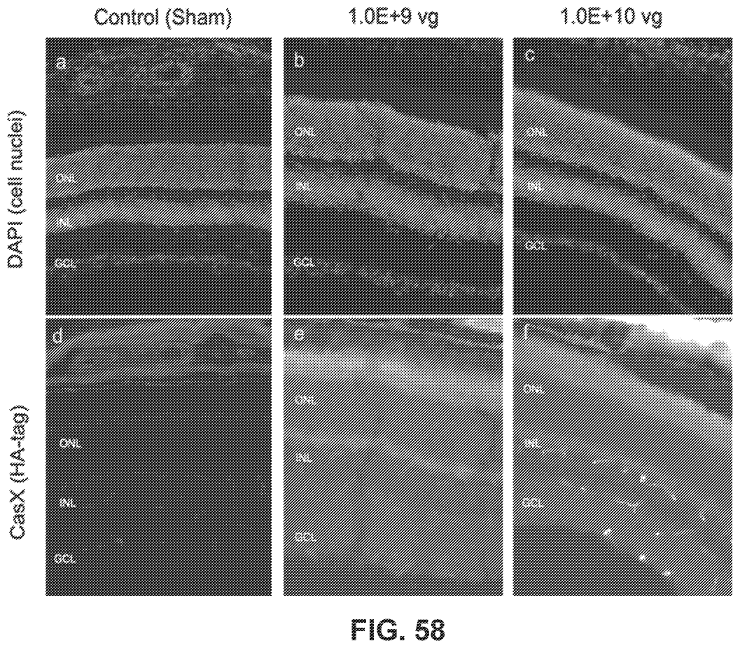

Immunohistochemistry performed on injected retinal cross-sectioned confirmed CasX expression in the photoreceptor layers, but also showed spread of the virus to the inner layers as show in in FIGS. 58A-53F. The treatment groups were 1.0e+9 vg of AAV-CasX (FIGS. 58B and 58E); 1.0e+10 vg AAV-CasX (FIGS. 58C and 58F); or PBS (FIGS. 58A and 58D). Levels of HA-tagged CasX was assessed by Anti-HA antibody staining (lower panels of FIGS. 53E, and 53F) in the photoreceptor cell bodies in the located in the outer nuclear layer (ONL) as well as outer segments, in retinas injected with both the 1e9 vg (FIGS. 58B and 58E) and 1e10 vg (FIGS. 58C and 58F). The control retinas that received a sham (FIGS. 58A and 59C) injection only showed background levels of signal for HA staining (FIG. 58D) in the RPE/sclera and had no detectable level in the ONL/INL layer. Additionally, gross histological analysis showed that the retinal structure was maintained after subretinal administration of AAV packaging CasX constructs.

Under the conditions of the experiments, the results demonstrate proof-of-concept that CasX 491, scaffold 174, and a spacer targeting the mouse P23 RHO locus can achieve therapeutically-relevant levels of edits at the P23 mouse locus when sub-retinally delivered via AAV in the murine retina.

### Example 16: AAV-Mediated Selective Expression of CasX in Photoreceptors Result in Strong On-Target Activity In Vivo by NGS and Structural Analysis

Experiments were conducted to demonstrate the ability of CasX to edit selectively photoreceptors in the mouse retina by restricting its expression with a selective photoreceptor promoter, with a spacer targeting the P23 residue at a therapeutically relevant level in the wild-type retina. Further, a strong correlation between editing and proteomic levels was shown in a transgenic reporter model expressing GFP only in rod photoreceptors. Here, it was assessed whether CasX variant 491 and guide variant 174 with a spacer targeting the integrated GFP locus generated significant, detectable editing levels in the retina when injected sub-retinally, and evaluated the efficacy of two different viral doses (1.0e+9 and 1.0e+10 vg per eye).

### Methods

Generation of AAV Plasmids and Viral Vectors: The CasX variant 491 under the control of the various photoreceptor-specific promoters (RP1, RP2, RP3 based on endogenous rhodopsin RHO promoter, and RP4, RP5 based on endogenous G-coupled Retinal Kinase GRK1 promoter; sequences in Table 27) as well as the CMV promoter, and the gRNA guide variant 174/spacer 11.30 (AAGGGGCUCCGCACCACGCC; SEQ ID NO: 9340), targeting mouse RHO exon 1 at P23 residue) under the U6 promoter were cloned into pAAV plasmid flanked with AAV2 ITR. A WPRE sequence was also included in the p59.RP4.491.174.11.30, and p59.RP5.491.174.11.30 plasmids. For the efficacy study in the Nrl-GFP model, spacer 4.76 (UGUGGUCGGGGUAGCGGCUG; SEQ ID NO: 9341) targeting GFP was cloned into AAV-cis plasmid p59.RP1.491.174 using the standard cloning methods.

TABLE 27
Rhodopsin promoter sequences

| | PR | SEQ |
|---|---|---|
| Promoter | construct | ID NO: |
| RHO | RP1 | 3714 |
| RHO535-CAG | RP2 | 3715 |
| RHO-intron | RP3 | 3716 |
| GRK1 | RP4 | 3717 |
| GRK1-SV40 | RP5 | 3718 |
| GRK1-CAG | RP6 | 3719 |

AAV vector production and titering were performed as described in Example 1.

The AAV vector AAV.RP1.491.174.4.76 was produced at the University of North Carolina (UNC) Vector Core using the triple transfection methods in HEK239T.

### Subretinal Injections:

C57BL/6J mice and heterozygous Nrl-GFP/C57BL/5J mice (Jackson Laboratories) were maintained in a normal 12-hour light/dark cycle. Subretinal injections were performed on 4-5 week-old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with Genteal® gel during the surgery. Under a surgical microscope, an ultrafine 30½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each mouse from the experimental groups was injected in one eye with 1.0e+9, 5.0e+9 or 1.0e+10 genome (vg)/eye, and the contralateral eye injected with the AAV formulation buffer.

### Western Blot:

To generate protein lysates, eyes were freshly enucleated and dissected in ice-cold PBS, snap-frozen in dry ice, and resuspended in RIPA buffer (150 mM NaCl, 1% NP40, 0.5% deoxycholate, 0.1% SDS, 50 mM Tris pH8.0, dH20) freshly supplemented with protease inhibitors (5 mg/mL final concentration), DTT and PMSF (final concentration 1 mM respectively) in individual 1.5 mL Eppendorf tube per retina. Retinal tissue was further homogenized in small pieces using an RNA-free disposable pellet pestles (Fisher scientific, #12-141-364) and incubated on ice for 30 minutes, flipping the tube occasionally to gently mix. Samples were then centrifuged at 4° C. at full speed for 20 minutes to pellet genomic DNA. Protein extracts and gDNA cell pellets were then separated. For protein extracts, supernatants were collected. Protein concentrations were determined by BCA assay and read on Tecan plate reader. 15 μg of total protein lysate of mouse retina were separated by SDS-PAGE (Bio-Rad TGX gels) and transferred to polyvinylidene difluoride membranes using the Transblot Turbo. The membranes were blocked with 5% nonfat dry milk for 1 hour at room temperature and incubated overnight at 4° C. with the primary antibody. Then, blots were washed with Tris-buffered saline with the Tween-20 (137 mM sodium chloride, 20 mM Tris, 0.1% Tween-20, pH 7.6) for three times and incubated with the horseradish peroxidase-conjugated anti-rabbit or anti-mouse secondary antibody for 1 hour at room temperature. After washing three times, the membranes were developed using Chemiluminescent substrate ECL and imaged on the ChemicDoc™. Blot images were processed with ImageLab.

### Tissue Processing and NGS Analysis:

Animals were sacrificed and the eyes enucleated in fresh PBS. Whole retinae were isolated from the eye cups and processed for gDNA extraction as described previously in western blot section. Genomic gDNA pellets were processed with the DNeasy® Blood & Tissue Kit (Qiagen®) according to the manufacturer's instructions. Amplicons were amplified from 200 ng of gDNA with a set of primers targeting the genomic region of interest. Amplicons were bead-purified (Beckman coulter, Agencourt Ampure XP) and then re-amplified to incorporate Illumina™ adapter sequence. Specifically, these primers contained an additional sequence at the 5′ ends to introduce Illumina™ read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq™ according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Immunohistology:

Enucleated eyes were placed in 10% formalin overnight at 4° C. Retinae were dissected out from the eye cups, rinsed in PBS thoroughly and immersed in 15%-30% sucrose gradient. Tissues were embedded in optimal cutting temperature (OCT), frozen on dry ice before being transferred to −80′C storage. 20 μM sections were cut using a cryostat. The sections were blocked for ≥1 hour at room temperature in the blocking buffer (2% normal goat serum, 1% BSA, 0.1% Triton-X 100) before antibody labeling. The antibodies used were: anti-mouse HA (Abcam, 1:500); Alexa Fluor 488 rabbit anti-mouse (Invitrogen™, 1:2000). Slides were counterstained with Hoechst 33342 (Thermo Fisher Scientific™, Hemel Hempstead, UK) and mounted with Prolong Diamond antifade mounting medium (Thermo Fisher Scientific™, Hemel Hempstead, UK). Confocal fluorescence imaging was subsequently performed using the LSM-710 inverted confocal microscope system (Carl Zeiss, Cambridge, UK).

### Results

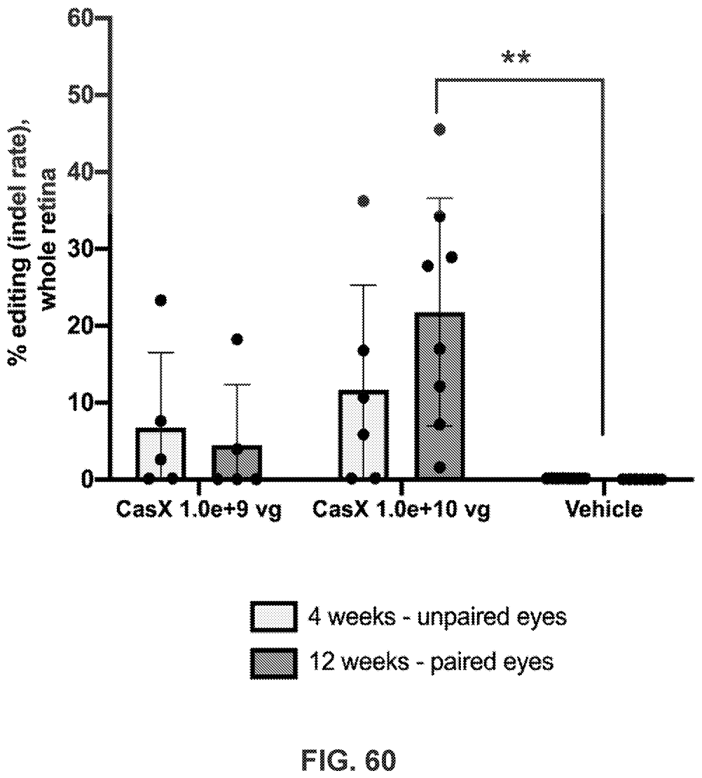

Editing levels were quantified at the mRHO exon locus in 3 week-old C57BL/6J that were injected sub-retinally with AAV vectors expressing CasX 491 under the control of multiple engineered retinal and ubiquitous promoters to identify promoters driving strong levels of editing in the photoreceptors, with spacer 11.30. Rod-specific RP1, RP2, RP3, RP4 promoters mediated very similar levels of editing (˜20%). Vectors AAV.RP5.491.174.11.30 and AAV RP5.491.WPRE.174.11.30 led to lower expression levels (˜10 and 8% respectively, FIG. 59A). Optimized vectors AAV.RP1.491.174.11.30 were identified as the most potent vectors for further functional and distribution study, with the goal of achieving high levels of editing in vivo in photoreceptors as well as making the transgene plasmid significantly smaller in size to package within the AAV (100-400 bp shorter than other constructs with similar level of activity (FIG. 59B). This optimized construct was further validated by conducting an efficacy study in a transgenic model expressing GFP in rod photoreceptors, a convenient model used in the field to validate rod-specific or knock down of protein. AAV.RP1.491.174.4.76 vectors were injected at 2 different doses to study efficacy. 4 and 12-weeks post-injections, editing levels at the integrated GFP locus were quantified by NGS, and detectable editing levels were observed. With the 1.0E+9 vg/eye dose arm, ˜8% of editing levels were observed. With the increased dose group injected with 1.0e+10 vg, 10% editing levels were detectable at 4 weeks, which increased by 2-fold in the follow-up time point, 12 weeks post-injections (FIG. 60).

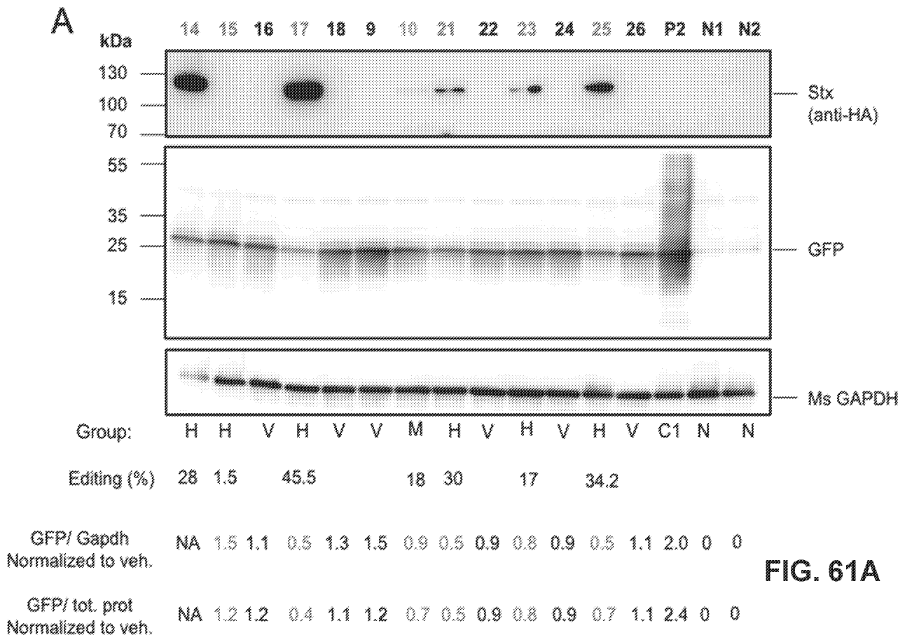

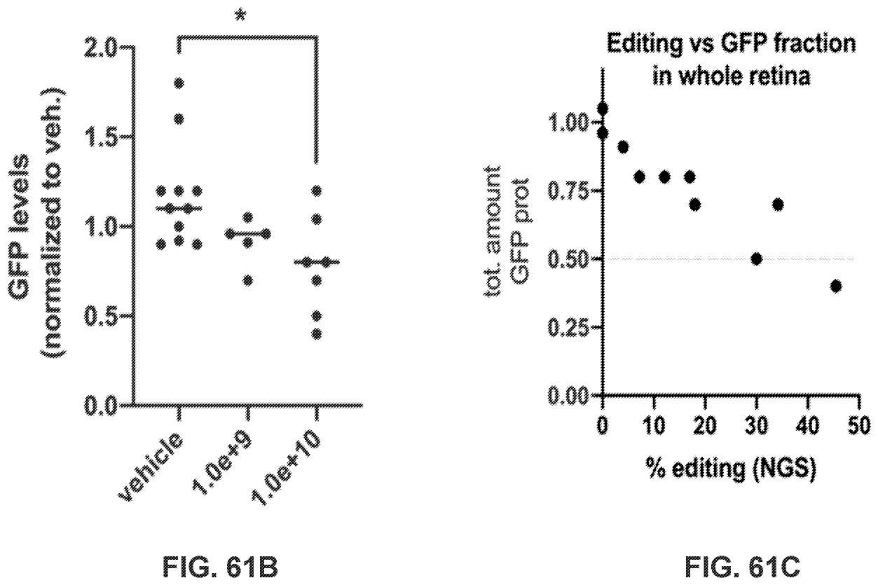

Editing levels were confirmed by structural and proteomic analysis. Western blot analysis of 12-week post-injection retinal lysates showed strong correlation between levels of editing and reduction in GFP protein (FIGS. 61A and 61C), with protein knock-down detected with as low as 5% editing in whole-retina. GFP protein levels were significantly lower than the vehicle group in the AAV-CasX-treated retinas at the 1.0e+10 vg/eye dose (FIG. 61).

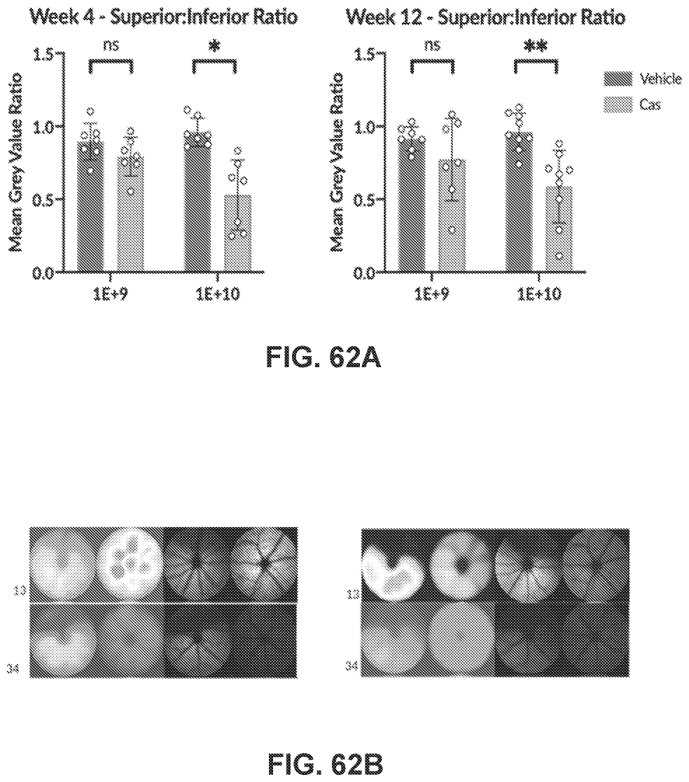

These results were also confirmed by in vivo fundus imaging of GFP fluorescence. The ratio of superior to inferior retina mean grey values showed a reduction in 20% and 50% GFP fluorescence by week 12 (FIG. 62A). A complete decrease in GFP fluorescence over time was visible within the quadrant who received the subretinal injection only in the injected retinas compared to the vehicle group (FIG. 62B).

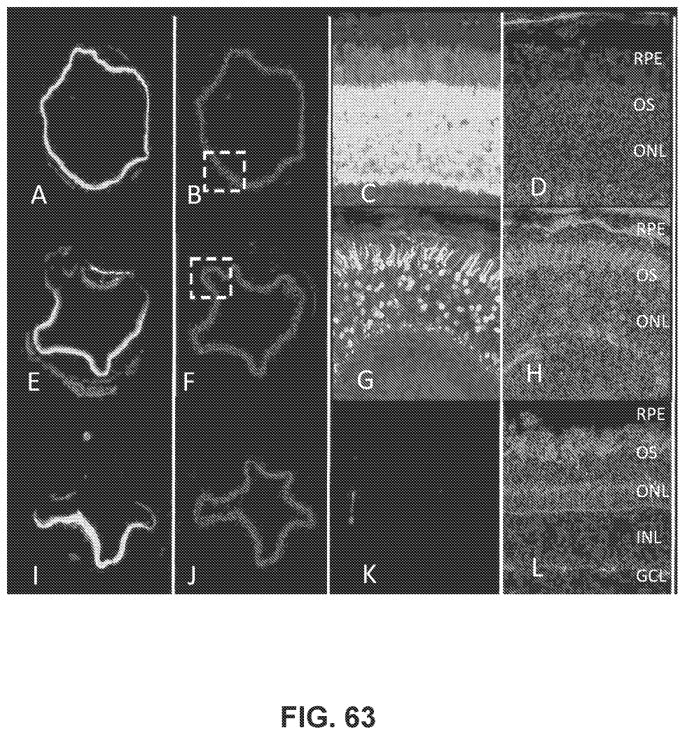

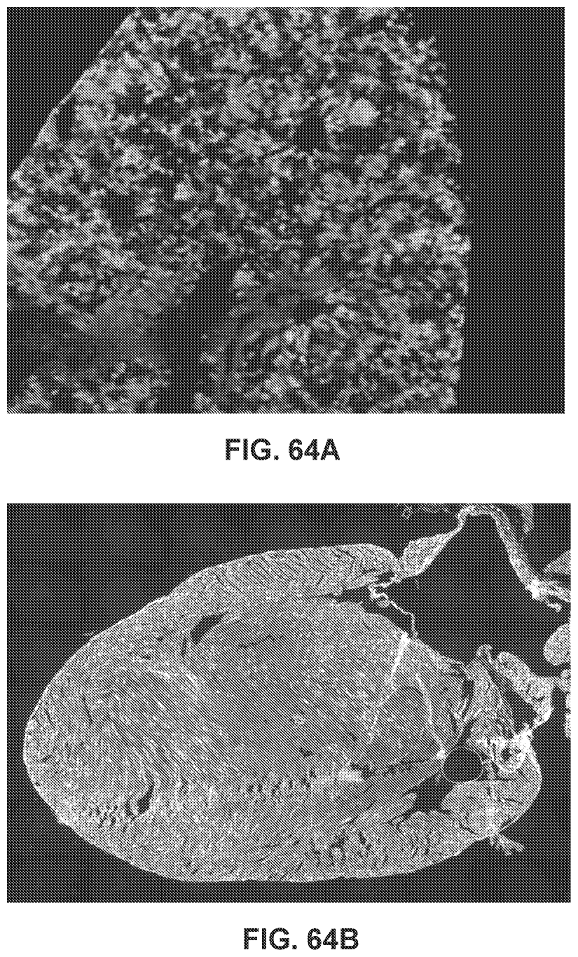

Immunochemistry staining confirmed (FIG. 63) the decrease of GFP protein expression in rod photoreceptors. Representative confocal images show strong GFP expression in the retinae injected with only the AAV formulation buffer. Whole retina is expressing GFP, matching with the nuclei staining (panels A-C of FIG. 63). No HA expression was detectable, as a read-out of AAV-mediated CasX transgene expression (panel D of FIG. 63). Retinae injected with 1.0e+9 and 1.0e+10 showed strong decrease in GFP expression in whole retina sections, in a dose-dependent manner (panels E-L of FIG. 63), which correlated with detectable levels of HA only rod outer segments (OS) and outer nuclear layers (ONL), confirming the promoter RP1 selectivity for rod photoreceptors. High dose treatment resulted in complete knockdown of injected retina (˜50% of GFP knockdown in whole-retina, as injection is limited to the superior gradient) while the 1.0e+9vg dose decreased ˜50% of GFP expression in localized area (panels G and K of FIG. 63) compared to control (panel C of FIG. 63).

The results demonstrate proof-of-concept that CasX with a gRNA targeting the mouse P23 RHO locus can achieve therapeutically-relevant levels of editing at the mouse P23 locus when only expressed in rod-photoreceptors, the therapeutic cell target, via AAV-mediated subretinal delivery. Furthermore, the specificity and efficacy of the vector were demonstrated by conducting a follow-up study targeting a GFP locus integrated in a reporter model overexpressing GFP in photoreceptors in which the results show a strong correlation between editing levels and protein knock-down assessed by western blot, fundus imaging and histology.

### Example 17: Demonstration that the CasX:gNA System can Edit Human Neural Progenitor Cells and Induced Neurons Efficiently when Packaged and Delivered Via AAVs

Experiments were performed to demonstrate the efficiency of AAV-expressed CasX:gNA system in editing human neural progenitor cells (hNPCs) and induced neurons (iNs) in vitro.

### Materials and Methods

### AAV Construct Cloning:

CasX variant 491 and guide scaffold variant 235 were used in these experiments.

To evaluate the editing capability of AAV-expressed CasX:gNA system in hNPCs, AAV constructs containing a UbC promoter driving CasX expression and a Pol III promoter scaffold driving the expression of a gRNA with scaffold variant 235 and spacer 7.37 (GGCCGAGAUGUCUCGCUCCG; SEQ ID NO: 4059; incorporated in construct ID 183), which targeted the endogenous B2M locus, were generated using standard molecular cloning techniques. Cloned and sequence-validated constructs were maxi-prepped and subjected to quality assessment prior to transfection for AAV production.

For experiments assessing the editing capability of AAV-expressed CasX:gNA system in human iNs, AAV constructs encoding for CasX protein and gRNA with AAVS1-targeting spacer 31.12 (UUCUCGGCGCUGCACCACGU; SEQ ID NO: 4060; incorporated in construct ID 188), 31.63 (CAAGAGGAGAAGCAGUUUGG; SEQ ID NO: 4061; incorporated in construct ID 189), or 31.82 (GGGGCCUGUGCCAUCUCUCG; SEQ ID NO: 4062; construct ID 190), were similarly generated as described. The non-targeting spacer 0.1 (AGGGGUCUUCGAGAAGACCC; SEQ ID NO: 4063) was also used in these experiments. For experiments assessing various protein promoters driving the expression of CasX 491 with gRNA spacer 7.37 to edit the B2M locus in human iNs, AAV constructs containing these protein promoter variants were similarly generated as described (see Table 28 for sequences of protein promoter variants). The sequences of the additional components of the AAV constructs, except for sequences encoding the CasX protein (Table 26), are listed in Table 45.

TABLE 28
Sequences of protein promoter variants, construct IDs of AAV constructs
that comprise each respective protein promoter variant, and SEQ
ID NOs for the sequences of each protein promoter variant.

| Promoter | Construct | SEQ | Length of |
|---|---|---|---|
| variant | ID | ID NO: | Promoter (bp) |
| UbC | 183 | 3720 | 400 |
| Jet | 191 | 3721 | 164 |
| U1a | 177 | 3722 | 252 |
| MeP426 | 192 | 3723 | 229 |
| miniCMV | 193 | 3724 | 38 |
| SFCp | 194 | 3725 | 354 |
| miniSV40 | 195 | 3726 | 197 |
| pJB42CAT5 | 196 | 3727 | 178 |
| MLP | 197 | 3728 | 41 |
| CMV core | 198 | 3729 | 204 |
| EFS | ND | 3730 | 234 |
| miniEF1α | ND | 3731 | 212 |
| hRPL30 | ND | 3732 | 325 |
| hRPS18 | ND | 3733 | 243 |
| hRPL13a | ND | 3734 | 223 |

*ND = no description.

### AAV Production:

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free Opti-MEM media. Three days later, cultures were centrifuged to separate the supernatant from the cell pellet, and the AAV particles were collected, concentrated, and filtered following standard procedures.

To determine the viral genome (vg) titer, 1 μL from crude lysate viruses was digested with DNase and proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples.

Culturing hNPCs In Vitro:

Immortalized hNPCs were cultured in hNPC medium (DMEM/F12 with GlutaMax™, 10 mM HEPES, 1×NEAA, 1×B-27 without vitamin A, 1×N2 supplemented growth factors hFGF and EGF, Pen/Strep, and 2-mercaptoethanol). Prior to testing, cells were lifted with TrypLE, gently resuspended to dissociate neurospheres, quenched with media, spun down, and resuspended in fresh media. Cells were counted and directly seeded at a density of ˜10,000 cells per well on a 96-well plate coated with PLF (poly-DL-omithine hydrobromide, laminin, and fibronectin) 24 hours prior to AAV transduction.

AAV Transduction of hNPCs, Followed by HLA Immunostaining and Flow Cytometry:

˜7,000 cells/well of hNPCs were seeded on PLF-coated 96-well plates. 24 hours later, seeded cells were treated with AAVs expressing the CasX:gRNA system. All viral infection conditions were performed at least in duplicate, with normalized number of viral genomes (vg) among experimental vectors, in a series of three-fold serial dilution of MOI ranging from 1E4 to 1E6 vg/cell. Five days post-transduction, AAV-treated hNPCs were lifted with TrypLE. After cell dissociation, staining buffer (3% fetal bovine serum in dPBS) was used for quenching. The dissociated cells were transferred to a round-bottom 96-well plate, followed by centrifugation and resuspension of cell pellets with staining buffer. After another centrifugation, cell pellets were resuspended in staining buffer containing the antibody (BioLegend) that would detect the B2M-dependent HLA protein expressed on the cell surface. After HLA immunostaining, cells were stained with DAPI to label cell nuclei. HLA+hNPCs were measured using the Attune NxT flow cytometer. Decreased or lack of HLA protein expression would indicate successful editing at the B2M locus in these hNPCs. A subset of transduced hNPCs were also lifted for genomic DNA extraction and editing analysis via next-generation sequencing (NGS).

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 200 ng of extracted gDNA with a set of primers specific to the target locus, such as the human B2M locus. These gene-specific primers contain an additional sequence at the 5′ end to introduce an Illumina™ adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

Reprogramming of Induced Pluripotent Stem Cells (iPSCs):

Fibroblast cells from a patient were obtained from the Coriell Cell Repository. iPSCs were generated from these lines by episomal reprogramming and genetically engineered to ectopically express Neurogenin 2 (Neurog2) to accelerate neuronal differentiation. Three iPSC clones were selected for downstream experiments.

### Neuronal Cell Culture:

All neuronal cell culture was performed using N2B27-based media. To induce neuronal differentiation, iPSCs were plated in neuronal plating media (N2B27 base media with 1 g/mL doxycycline, 200 μM L-ascorbic acid, 1 μM dibutyryl cAMP sodium salt, 10 M CultureOne, 100 ng/ml of BDNF, 100 ng/ml of GDNF). iNs were dissociated, aliquoted, and frozen for long term storage after three days of differentiation (DIV3). DIV3 iNs were thawed and seeded on a 96-well plate at 30,000 cells per well. iNs were cultured for one week in plating media and thereafter, half-media changes were performed once every week using feeding media (N2B27 base media with 200 μM L-ascorbic acid, 1 μM dibutyryl cAMP sodium salt, 200 ng/ml of BDNF, 200 ng/ml of GDNF).

### AAV Transduction of iNs In Vitro:

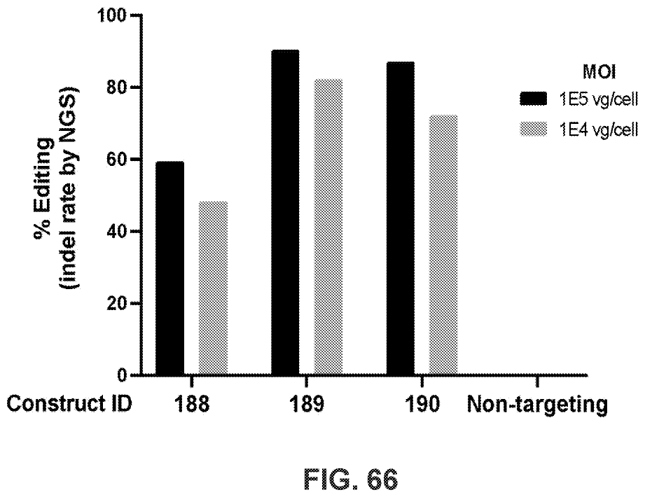

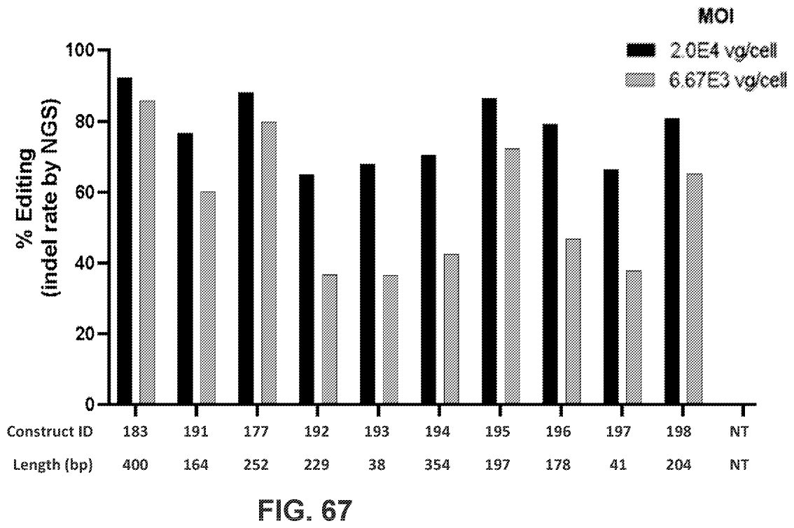

24 hours prior to transduction, -30,000-50,000 iNs per well were seeded on Matrigel-coated 96-well plates. AAVs expressing the CasX:gRNA system were then diluted in neuronal plating media and added to cells, with six wells per condition used as replicates. Cells were transduced at various MOIs (1E4 or 1E5 vg/cell for FIG. 66; 2E4 or 6.67E3 for FIG. 67). Seven days post-transduction, iNs were replenished using feeding media. 14 days post-transduction, cells were lifted using lysis buffer, 6-well replicates were pooled, and gDNA was harvested and prepared for editing analysis at either the human AAVS1 or B2M locus using NGS.

### Results

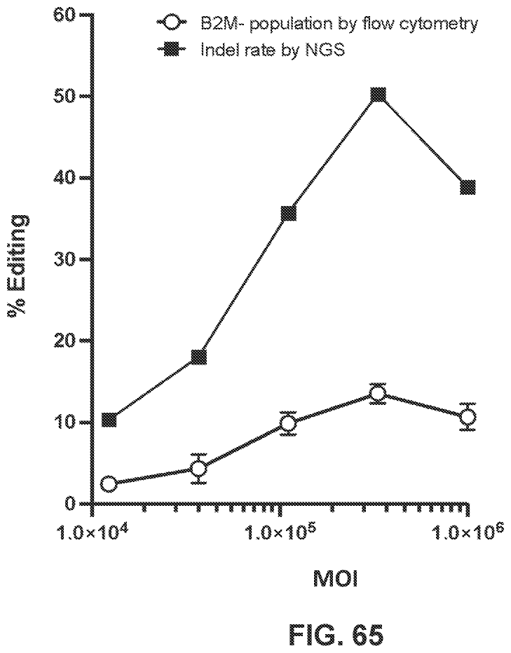

FIG. 65 shows the quantification of percent editing at the B2M locus measured via two different assessments (as indel rate quantified genotypically by NGS and as a phenotypic readout B2M-cell population detected by flow cytometry) in human NPCs five days post-transduction with AAVs at various MOIs. Efficient editing at the human B2M locus was observed, with the highest level of editing achieved at the MOI of ˜3E5: ˜50% indel rate and ˜13% of cells exhibiting the B2M protein knockout phenotype. FIG. 66 also illustrates efficient editing at the AAVS1 locus in human iNs, with construct ID 189 achieving ˜90% editing at the higher MOI of 1E5. As expected, no editing was observed at the AAVS1 locus with the non-targeting spacer.

FIG. 67 shows that robust editing at the B2M locus was achieved for several of the various protein promoters used to drive expression of CasX variant 491. Briefly, AAVs were generated with the indicated transgene constructs and transduced into human iNs at either an MOI of 2E4 or 6.67E3. AAV constructs 177 and 183 contained promoters that demonstrated the highest editing activity, with at least 80% efficiency at either MOI.

The results of these experiments demonstrate that CasX variant 491 and guide scaffold 235 with spacer targeting either the human B2M locus or the human AAVS1 locus can edit on-target efficiently when packaged and delivered in vitro via AAVs into human NPCs or iNs.

### Example 18: CpG-Depleted AAVs Demonstrate CasX-Mediated Editing In Vitro

Pathogen-associated molecular patterns (PAMPs) such as unmethylated CpG motifs are small molecular motifs conserved within a class of microbes. They are recognized by toll-like receptors (TLRs) and other pattern recognition receptors in eukaryotes and often induce a non-specific immune activation. In the context of gene therapy, therapeutics containing PAMPs are often not as well-tolerated and are rapidly cleared from the patient given the strong immune response triggered, which ultimately leads to reduced therapeutic efficiency. As a result, there is an unmet need for well-tolerated gene therapy vectors that are not cleared rapidly to achieve the necessary therapeutic benefit.

CpG motifs are short single-stranded DNA sequences containing the dinucleotide CG. When these CpG motifs are unmethylated, they act as PAMPs and therefore potently stimulate the immune response. In this example, experiments were performed to deplete CpG motifs in the AAV construct encoding CasX variant 491, guide scaffold variant 235, and spacer 7.37 targeting the endogenous B2M(beta-2-microglobulin) locus to demonstrate that CpG-depleted AAV vectors can edit effectively in vitro. The editing activity induced from use of the individual elements of the AAV genome and their respective CpG-reduced versions, as well as combinations of these elements, was assessed in vitro. In vitro assessment of immunogenicity is presented in Example 19.

### Materials and Methods

### Design of CpG-Depleted AAV Components:

Nucleotide substitutions to replace native CpG motifs in AAV components were designed in silico. For exemplary regulatory elements, nucleotide substitutions to replace native CpG motifs were designed based on homologous nucleotide sequences from related species to produce CpG-reduced variants for the following elements: the murine U1a snRNA (small nuclear RNA) gene promoter, the human UbC (polyubiquitin C) gene promoter, and the human U6 promoter. See Table 29, which provides parental sequences of a murine U1a promoter, a human UbC promoter, and a human U6 promoter prior to CpG reduction and Table 30, which provides sequences of CpG-reduced variants of the promoters listed in Table 29. Similar modifications were made to produce a CpG-reduced variant of a bGHpA (bovine growth hormone polyadenylation) sequence. See Table 31, which provides a parental sequence of a bGHpA prior to CpG reduction and Table 32, which provides a sequence of a CpG-reduced variant of the bGHpA listed in Table 31.

AAV2 ITRs were CpG-depleted as previously described (Pan X, Yue Y, Boftsi M. et al., 2021, Rational engineering of a functional CpG-free ITR for AAV gene therapy. Gene Ther.) See Table 33, which provides parental ITR sequences prior to CpG reduction and Table 34, which provides sequences of CpG-reduced variants of the ITRs listed in Table 33.

Nucleotide substitutions to replace native CpG motifs in exemplary Cas protein variants (CasX variants) were rationally designed with codon optimization, so that the amino acid sequence of the CpG-reduced Cas-encoding sequence would be the same as the amino acid sequence of the corresponding native Cas-encoding sequence. See Table 35, which provides parental Cas sequences prior to CpG reduction and Table 36, which provides sequences of CpG-reduced variants of the Cas proteins listed in Table 35. Furthermore, nucleotide substitutions to replace native CpG motifs within the base gRNA scaffold variants (gRNA scaffold 235 and 316) were rationally designed with the intent to preserve editing activity. The rational design process for the CpG reduction of the gRNA sequences is further described herein below. See Table 37, which provides parental gRNA sequences prior to CpG reduction and Table 38, which provides sequences of CpG-reduced variants of the gRNAs listed in Table 37.

All resulting sequences were ordered from a third-party commercial source as synthesized gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 4059), which targets the endogenous B2M gene, was used for the relevant experiments discussed in this example. The resulting AAV constructs were generated using standard molecular cloning techniques. Cloned and sequence-validated plasmid constructs were midi-prepped for subsequent nucleofection and AAV vector production.

TABLE 29
Parental sequences of promoters

| | SEQ |
|---|---|
| Parental element | ID NO |
| Parental UbC promoter (human) | 3533 |
| Parental U1A promoter (murine) | 3722 |
| Parental U6 promoter (human) | 3563 |

TABLE 30
Sequences of CpG-reduced or depleted promoters

| | AAV | SEQ |
|---|---|---|
| CpG-reduced or depleted element | construct ID | ID NO |
| CpG-reduced UbC promoter (human) | 184 | 3735 |
| Strongly CpG-reduced UbC promoter | 185 | 3736 |
| (human) | | |
| CpG-depleted UbC promoter (human) | 186 | 3737 |
| CpG-reduced U1a promoter (murine) | 178, 206 | 3738 |
| CpG-depleted U1a promoter (murine) | 179, 205 | 3739 |
| CpG-reduced U6 promoter (human) | 180 | 3740 |
| CpG-depleted U6 promoter (human) | 181, 205, 206 | 3741 |
| CpG-reduced hU6 Isoform 2 | — | 3742 |
| CpG-depleted hU6 Isoform 2 | — | 4743 |
| CpG-depleted hU6 Isoform 3 | — | 3744 |
| CpG-depleted hU6 Isoform 4 | — | 3745 |
| CpG-depleted hU6 Isoform 5 | — | 3746 |

TABLE 31
Parental sequence for Poly(A) signal sequence

| Parental element | SEQ ID NO |
|---|---|
| Parental bGH-polyA | 3401 |
| sequence (bovine) | |

TABLE 32
Sequences of CpG-reduced Poly(A) signal sequence

| CpG-reduced or depleted element | AAV construct ID | SEQ ID NO |
|---|---|---|
| CpG-depleted bGH-polyA sequence | 182, 205, 206 | 3748 |
| (bovine) | | |

TABLE 33
Sequences of parental AAV ITR sequences

| Parental element | SEQ ID NO |
|---|---|
| 5′ITR | 17 |
| 3′ITR | 3701 |

TABLE 34
Sequences of CpG-reduced or depleted AAV ITR sequences

| CpG-reduced or depleted element | SEQ ID NO |
|---|---|
| CpG-depleted 5′ITR | 3749 |
| CpG-depleted 3′ITR | 3750 |

TABLE 35
Parental sequences of CasX proteins

| Parental element | SEQ ID NO: |
|---|---|
| CasX 491 | 9320 |
| CasX 515 | 9321 |
| CasX 676 | 9322 |
| CasX 593 | 9323 |
| CasX 812 | 9324 |
| CasX 668 | 9325 |
| CasX 672 | 9326 |

TABLE 36
CpG- depleted sequences of CasX proteins

| CpG- depleted element | AAV construct ID: | SEQ ID NO: |
|---|---|---|
| CpG-depleted CasX 491 | 205, 206 | 9327 |
| CpG-depleted CasX 515 | — | 9328 |
| CpG-depleted CasX 593 | — | 9329 |
| CpG-depleted CasX 812 | — | 9330 |
| CpG-depleted CasX 668 | — | 9331 |
| CpG-depleted CasX 676 | — | 9332 |
| CpG-depleted CasX 672 | — | 9333 |

TABLE 37
Parental sequences of gRNA scaffolds

| Parental element | SEQ ID NO: |
|---|---|
| Scaffold 235 | 9334 |
| Scaffold 316 | 9335 |

TABLE 38
Sequences of CpG-reduced or depleted gRNA scaffolds

| Scaffold ID: | Derived from parent scaffold: | SEQ ID NO: |
|---|---|---|
| Scaffold 320 | Scaffold 235 | 3751 |
| Scaffold 321 | Scaffold 235 | 3752 |
| Scaffold 322 | Scaffold 235 | 3753 |
| Scaffold 323 | Scaffold 316 | 3754 |
| Scaffold 324 | Scaffold 235 | 3755 |
| Scaffold 325 | Scaffold 235 | 3756 |
| Scaffold 326 | Scaffold 316 | 3757 |
| Scaffold 327 | Scaffold 235 | 3758 |
| Scaffold 328 | Scaffold 235 | 3559 |
| Scaffold 329 | Scaffold 316 | 3760 |
| Scaffold 330 | Scaffold 235 | 3761 |
| Scaffold 331 | Scaffold 235 | 3762 |
| Scaffold 332 | Scaffold 316 | 3763 |
| Scaffold 333 | Scaffold 235 | 3764 |
| Scaffold 334 | Scaffold 235 | 3765 |
| Scaffold 335 | Scaffold 316 | 3766 |
| Scaffold 336 | Scaffold 235 | 3767 |
| Scaffold 337 | Scaffold 235 | 3768 |
| Scaffold 338 | Scaffold 316 | 3769 |
| Scaffold 339 | Scaffold 235 | 3770 |
| Scaffold 340 | Scaffold 235 | 3771 |
| Scaffold 341 | Scaffold 316 | 3772 |

### Design of CpG-Depleted Guide Scaffolds:

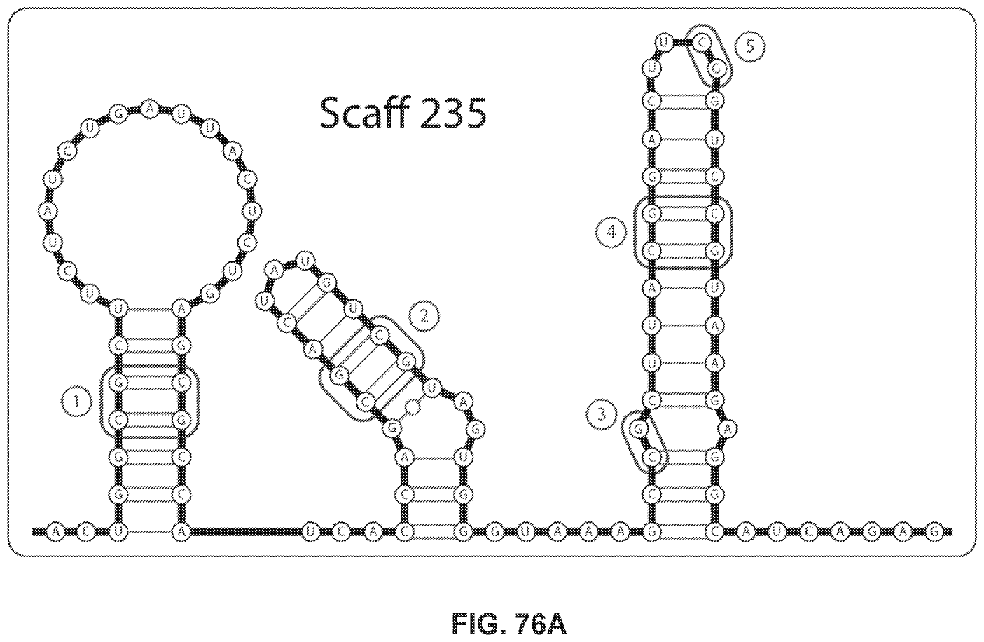

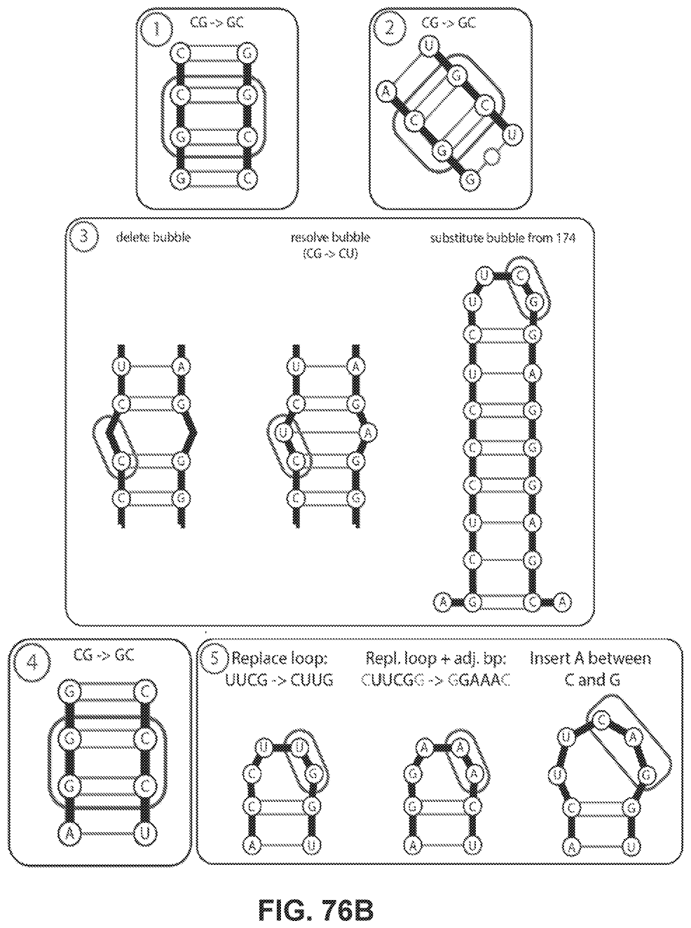

Nucleotide substitutions were rationally-designed to replace native CpG motifs within the base gRNA scaffold variant (gRNA scaffold 235) with the intent to preserve editing activity while reducing scaffold immunogenicity. CpG-motifs were removed from the scaffold coding sequence to reduce immunogenicity. Scaffold 235 contains a total of eight CpG elements; six of which are predicted to basepair and form complementary strands of a double-stranded secondary structure (FIG. 76A). Therefore, the six basepairing CpGs forming three pairs were mutated in concert to maintain these double-stranded secondary structures. These mutations reduced the count of independent CpG-containing regions to five (three CpG pairs and two single CpGs) to be considered independently for CpG-removal. Specifically, mutations were designed in (1) the pseudoknot stem, (2) the scaffold stem, (3) the extended stem bubble, (4) the extended step, and (5) the extended stem loop, as diagrammed in FIG. 76B and described in detail below.

In the pseudoknot stem (region 1), the CpG pair was flipped to a GpC to minimize the alteration of the base composition and sequence. Based on previous experiments involving replacing individual base pairs, it was anticipated that this mutation was not likely to be detrimental to the structure and function of the guide RNA scaffold.

Similarly, in the scaffold stem (region 2) the CpG pair was flipped to a GpC to minimize the alteration of the base composition and sequence. It was anticipated that this mutation was likely to be detrimental to the structure and function of the guide RNA scaffold because strong sequence conservation was seen in this region in previous experiments mutating individual bases or base pairs. This strong sequence conservation is likely due to the scaffold stem loop being important in interacting with the CasX protein as well as in the formation of a triplex structural element with the pseudoknot region.

In the extended stem bubble (region 3) the single CpG was removed by one of three strategies. First, the bubble was deleted by mutating CG->C (removing the guanine from the CpG dinucleotide). Second, the bubble was resolved to restore ideal basepairing by mutating CG->CT (substituting thymine for guanine in the CpG dinucleotide). Third, the entire extended stem loop was replaced with the extended stem loop of scaffold 174. Note that, by itself, the replacement of the extended stem loop with that of scaffold 174 recapitulates scaffold 316, which has previously been shown to edit efficiently. There are no CpG motifs in the extended stem loop of scaffold 174. Therefore, replacing the extended stem loop with that of scaffold 174 also removes the CpG motif in the extended stem (region 4). Based on previous experiments showing the relative robustness of the extended stem to small changes, it was anticipated that mutating the extended stem bubble was moderately likely to be detrimental to the structure and function of the guide RNA scaffold.

In the extended stem (region 4), the CpG pair could not be flipped to GpC without generating additional CpG motifs. Therefore, the CpGs were changed to a GG and a complementary CC motif Similar to region 3, based on the relative robustness of the extended stem to small changes, it was anticipated that this mutation was not likely to be detrimental to the structure and function of the guide RNA scaffold.

Finally, the extended stem loop (region 5) was mutated in one of three ways that were designed based on previous experiments examining the stability of the stem loop. In particular, several variations of the stem loop had previously been shown to have similar stability levels, and some of these variations of the stem loop do not contain CpGs. Based on these findings, first, the loop was replaced with a new loop with a CUUG sequence. Second, the loop was replaced with a new loop with a GAAA sequence. Since the GAAA loop replacement would generate a novel CpG adjacent to the loop, it was combined with a C->G base swap and the corresponding G->C base swap on the complementary strand, ultimately resulting in a CUUCGG->GGAAAC exchange. Third, the loop was mutated by the insertion of an A to interrupt the CpG motif and thereby increase the size of the loop from 4 to 5 bases. It was anticipated that randomly mutating the extended stem loop would likely have detrimental effects on secondary structure stability and hence on editing. However, relying on previously confirmed sequences was believed to have a lower risk associated with a replacement.

To generate guide RNA scaffolds encoded by DNA with reduced CpG levels, the mutations described above were combined in various configurations. Table 39, below, summarizes combinations of the mutations that were used. In Table 39, a 0 indicates that no mutation was introduced to a given region, a 1, 2, or 3 indicates that a mutation was introduced in that region, as diagrammed in FIG. 76B, and n/a indicates not applicable. Specifically, for region 1, the pseudoknot stem, a 1 indicates that a CG->GC mutation was introduced. For region 2, the scaffold stem, a 1 indicates that a CG->GC mutation was introduced. For region 3, the extended stem bubble, a 1 indicates that the bubble was removed by the deletion of the G and A bases that form the bubble, a 2 indicates that the bubble was resolved by a CG->CT mutation that allows for basepairing between the A and T bases, and a 3 indicates that the extended stem loop was replaced with the extended step loop from guide scaffold 174. For region 4, the extended stem, a 1 indicates that a CG->GC mutation was introduced. For region 5, the extended stem loop, a 1 indicates that the loop was replaced from TTCG to CTTG, a 2 indicates that the loop was replaced along with a basepair adjacent to the loop, from CTTCGG to GGAAAC, and a 3 indicates that an A was inserted between the C and the G.

TABLE 39
Summary of mutations for CpG-reduction
and depletion in guide scaffold 235

| | | | Region 3 | | |
|---|---|---|---|---|---|
| | Region 1 | Region 2 | (Extended | Region 4 | Region 5 |
| Scaffold | (Pseudoknot | (Scaffold | stem | (Extended | (Extended |
| ID | stem) | stem) | bubble) | stem) | stem loop) |
| 320 | 1 | 0 | 0 | 1 | 0 |
| 321 | 1 | 0 | 1 | 1 | 0 |
| 322 | 1 | 0 | 2 | 1 | 0 |
| 323 | 1 | 0 | 3 | n/a | 0 |
| 324 | 1 | 0 | 1 | 1 | 1 |
| 325 | 1 | 0 | 2 | 1 | 1 |
| 326 | 1 | 0 | 3 | n/a | 1 |
| 327 | 1 | 0 | 1 | 1 | 2 |
| 328 | 1 | 0 | 2 | 1 | 2 |
| 329 | 1 | 0 | 3 | n/a | 2 |
| 330 | 1 | 0 | 1 | 1 | 3 |
| 331 | 1 | 0 | 2 | 1 | 3 |
| 332 | 1 | 0 | 3 | n/a | 3 |
| 334 | 1 | 1 | 2 | 1 | 1 |
| 335 | 1 | 1 | 3 | n/a | 1 |
| 336 | 1 | 1 | 1 | 1 | 2 |
| 337 | 1 | 1 | 2 | 1 | 2 |
| 338 | 1 | 1 | 3 | n/a | 2 |
| 339 | 1 | 1 | 1 | 1 | 3 |
| 340 | 1 | 1 | 2 | 1 | 3 |
| 341 | 1 | 1 | 3 | n/a | 3 |
| 235 | 0 | 0 | 0 | 0 | 0 |

Generation of CpG-Depleted AAV Plasmids to Assess CpG-Reduced or Depleted gRNA Scaffolds:

The CpG-reduced or depleted gRNA scaffolds were tested in the context of AAV vectors that were otherwise CpG-depleted, with the exception of the AAV2 ITRs. Specifically, nucleotide substitutions to replace native CpG motifs in AAV components were designed in silico based on homologous nucleotide sequences from related species for the following elements: the murine U1a snRNA (small nuclear RNA) gene promoter, the bGHpA (bovine growth hormone polyadenylation) sequence, and the human U6 promoter. The coding sequence for CasX 491 was optimized for CpG depletion. All resulting sequences (Tables 38 and 40) were ordered as gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 4059), which targets the endogenous B2M gene, was used for the experiments discussed in this example. The first time that the experiment was performed (“N=1”), a sample with the non-targeting spacer 0.0 was also included as a control (CGAGACGUAAUUACGUCUCG; SEQ ID NO: 9342).

The resulting AAV constructs were generated using standard molecular cloning techniques. Cloned and sequence-validated plasmid constructs were midi-prepped for subsequent nucleofection and AAV vector production. The sequences of the additional components of AAV constructs, with the exception of sequences encoding the gRNAs (Table 38), are listed in Table 40.

TABLE 40
Sequences of AAV elements (5′-3′ in AAV construct)

| Element | SEQ ID NO: |
|---|---|
| AAV2 5′ ITR | 17 |
| CpG-depleted U1a promoter | 3739 |
| CpG-depleted cMycNLS-CasX491-cMycNLS | 3747 |
| CpG-depleted bGH-polyA sequence | 3748 |
| CpG-depleted U6 promoter | 3741 |
| AAV2 3′ ITR | 3701 |

### AAV Production

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and encoding AAV rep/cap proteins using PEI MAX® (Polysciences) in serum-free Opti-MEM media. Three days later, cultures were centrifuged to separate the supernatant from the cell pellet, and the AAV particles were collected, concentrated, and filtered following standard procedures.

To determine the viral genome (vg) titer, 1 μL from crude lysate viruses was digested with DNase and proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples.

Culturing Human Neural Progenitor Cells (hNPCs) In Vitro:

Immortalized hNPCs were cultured in hNPC medium (DMEM/F12 with GlutaMax™, 10 mM HEPES, 1×NEAA, 1×B-27 without vitamin A, 1×N2 supplemented growth factors hFGF and EGF, Pen/Strep, and 2-mercaptoethanol). Prior to testing, cells were lifted with TrypLE, gently resuspended to dissociate neurospheres, quenched with media, spun down, and resuspended in fresh media. Cells were counted and directly used for nucleofection or are seeded at a density of ˜10,000 cells per well on a 96-well plate coated with PLF (poly-DL-ornithine hydrobromide, laminin, and fibronectin) 48 hours prior to AAV transduction.

Plasmid Nucleofection into Human Neural Progenitor Cells (hNPCs):

AAV plasmids encoding the CasX:gRNA system, with or without CpG depletion of the individual elements of the AAV genome, were nucleofected into hNPCs using the Lonza P3 Primary Cell 96-well Nucleofector Kit. Plasmids were diluted into two concentrations: 50 ng/μL and 25 ng/μL. 5 μL of DNA was mixed with 20 μL of 200,000 hNPCs in the Lonza P3 solution supplemented with 18% V/V P3 supplement. The combined solution was nucleofected using the Lonza 4D Nucleofector System following program EH-100. The nucleofected solution was subsequently quenched with the appropriate culture media and then divided into three wells of a 96-well plate coated with PLF. Seven days post-nucleofection, hNPCs were lifted for B2M protein expression analysis via HLA immunostaining followed by flow cytometry. Subsequently, stacking of individual CpG-depleted elements to create a combined AAV genome with substantial CpG depletion was performed and similarly tested for editing assessment at the B2M locus in vitro.

### Editing Activity Assessment by HLA Immunostaining and Flow Cytometry:

Seven days after nucleofection, AAV-treated hNPCs were lifted with TrypLE. After cell dissociation, staining buffer (3% fetal bovine serum in dPBS) was used for quenching. The dissociated cells were transferred to a round-bottom 96-well plate, followed by centrifugation and resuspension of cell pellets with staining buffer. After another centrifugation, cell pellets were resuspended in staining buffer containing the antibody (BioLegend) that would detect the B2M-dependent HLA protein expressed on the cell surface. After HLA immunostaining, cells were stained with DAPI to label cell nuclei. HLA+hNPCs were measured using the Attune NxT flow cytometer.

Reprogramming of Induced Pluripotent Stem Cells (iPSCs):

Fibroblast cells from a patient were obtained from the Coriell Cell Repository. iPSCs were generated from these lines by episomal reprogramming and genetically engineered to ectopically express Neurogenin 2 (Neurog2) to accelerate neuronal differentiation. Three iPSC clones were selected for downstream experiments.

### Neuronal Cell Culture:

All neuronal cell culture was performed using N2B27-based media. To induce neuronal differentiation, iPSCs were plated in neuronal plating media (N2B27 base media with 1 g/mL doxycycline, 200 μM L-ascorbic acid, 1 μM dibutyryl cAMP sodium salt, 10 μM CultureOne, 100 ng/ml of BDNF, 100 ng/ml of GDNF). iNs (induced neurons) were dissociated, aliquoted, and frozen for long term storage after three days of differentiation (DIV3). DIV3 iNs were thawed and seeded on a 96-well plate at ˜30,000-50,000 cells per well. iNs were cultured for one week in plating media and thereafter, half-media changes were performed once every week using feeding media (N2B27 base media with 200 μM L-ascorbic acid, 1 μM dibutyryl cAMP sodium salt, 200 ng/ml of BDNF, 200 ng/ml of GDNF).

### AAV Transduction of iNs in Vitro:

24 hours prior to transduction, ˜30,000-50,000 iNs per well were seeded on Matrigel-coated 96-well plates. AAVs expressing the CasX:gRNA system, with or without CpG depletion of the individual elements of the AAV genome, were then diluted in neuronal plating media and added to cells. Cells were transduced at two MOIs (1E3 or 3E3vg/cell). Seven days post-transduction, iNs were replenished using feeding media. Seven days post-transduction, cells were lifted using lysis buffer, 4-well replicates were pooled per experimental condition, and genomic DNA (gDNA) was harvested and prepared for editing analysis at the B2M locus using next generation sequencing (NGS). Subsequently, combining individual CpG-reduced or CpG-depleted elements to create a combined AAV genome with substantial CpG depletion was performed and similarly tested for editing assessment at the B2M locus in vitro. Experiments assessing the effects of incorporating CpG-depleted gRNA scaffold constructs on editing at the B2M locus in vitro are also conducted.

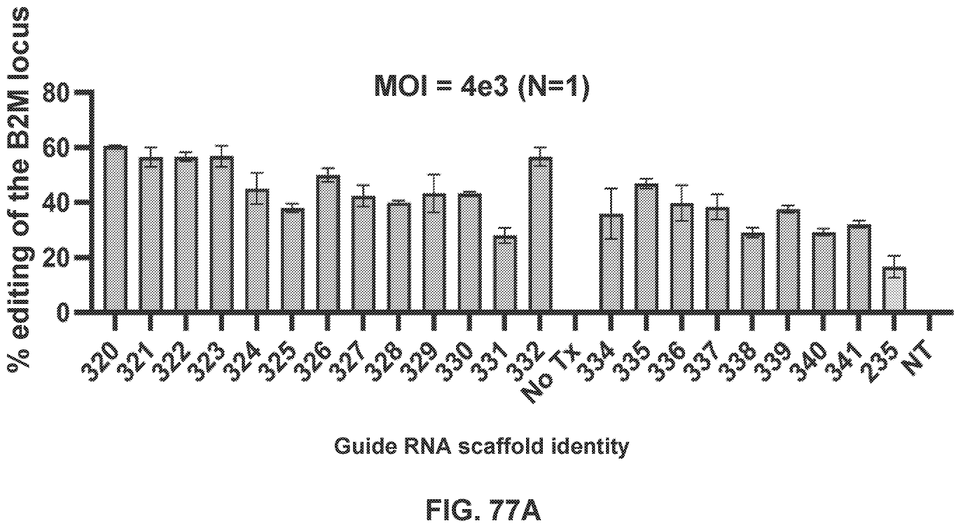

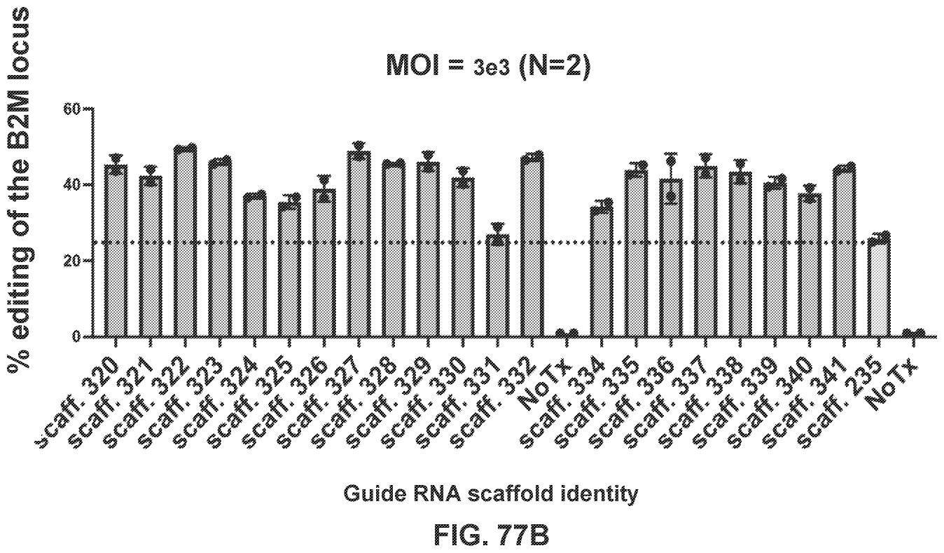

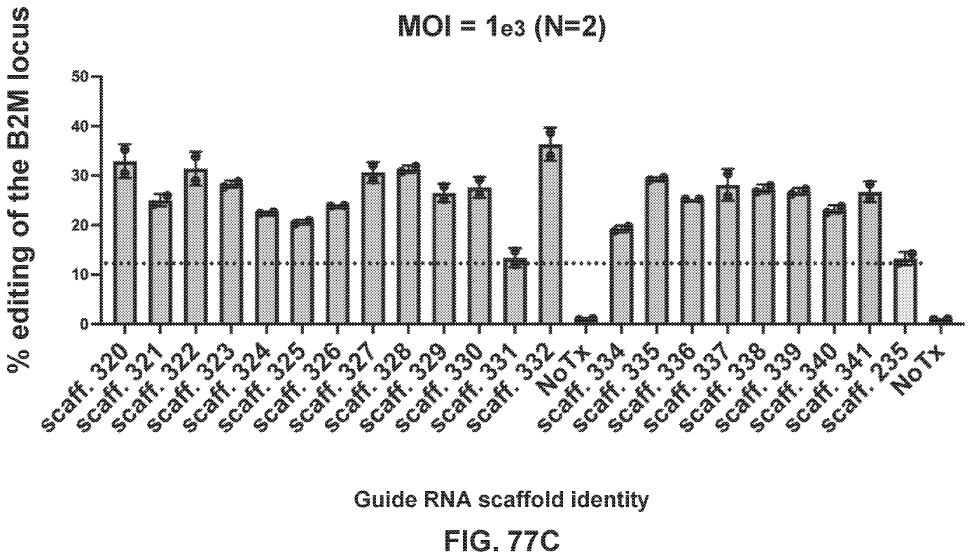

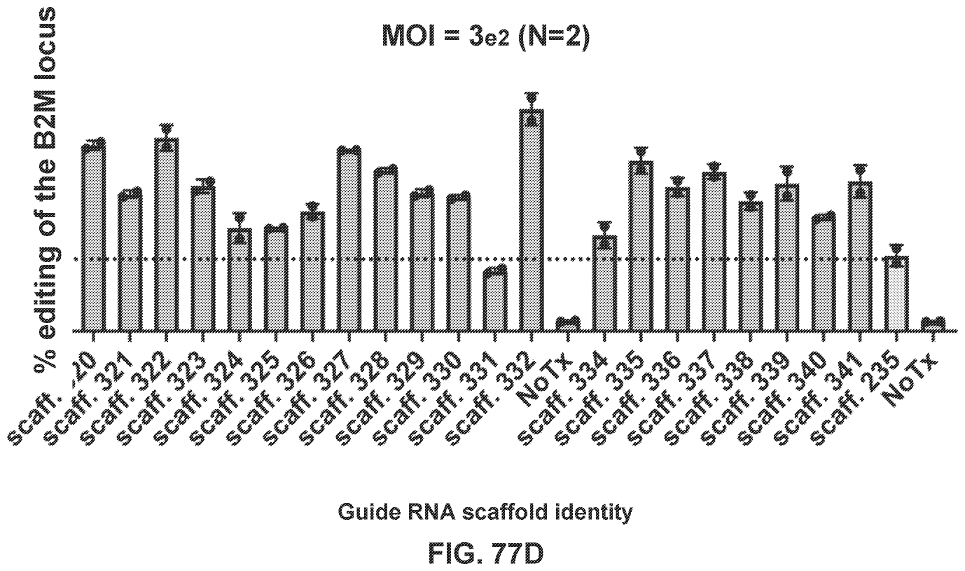

In a separate experiment, CpG-depleted guide scaffolds were assessed. Here, iNs were transduced with AAVs expressing the CasX:gRNA system with various versions of the guide scaffold. The first time that the experiment was performed (“N=1”), cells were transduced at an MOI of 4e3 vg/cell (see FIG. 77A). Seven days post-plating, iNs were transduced with virus diluted in fresh feeding media. Eight days post-transduction, cells were lifted using lysis buffer, 4-well replicates were pooled per experimental condition, and gDNA was harvested and prepared for editing analysis at the B2M locus using NGS. The second time that the experiment was performed (“N=2”), cells were transduced at an MOI of 3e3 vg/cell, 1e3 vg/cell, or 3e2 vg/cell (see FIG. 77B, FIG. 77C, and FIG. 77D. Seven days post-plating, induced neurons were transduced with virus diluted in fresh feeding media. Seven days post-transduction, cells were lifted using lysis buffer, 2-well replicates were pooled per experimental condition, and gDNA was harvested and prepared for editing analysis at the B2M locus using NGS. Samples that were not transduced with AAV were included as controls.

### NGS Processing and Analysis:

Genomic DNA (gDNA) from harvested cells was extracted using the Zymo Quick-DNA™ Miniprep Plus kit following the manufacturer's instructions. Target amplicons were formed by amplifying regions of interest from 200 ng of extracted gDNA with a set of primers specific to the target locus, such as the human B2M gene. These gene-specific primers contained an additional sequence at the 5′ end to introduce an Illumina™ adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Results

### Assessment of Use of CpG-Depleted AAV Vector Elements on Editing in a Cell-Based Assay:

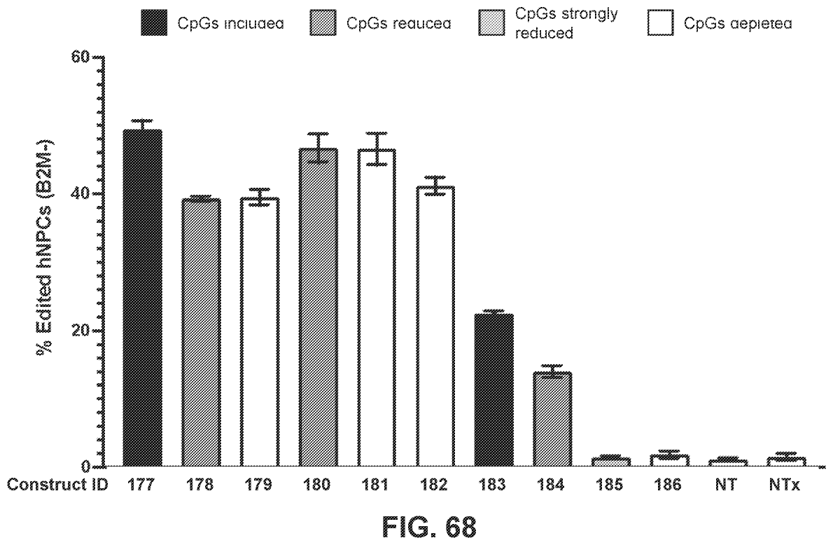

The findings of an assay assessing the editing activity at the B2M locus in hNPCs nucleofected with CpG-containing (CpG⁺) or CpG-reduced/depleted (CpG⁻) AAV vectors are illustrated in FIG. 68. Editing activity was measured as the percentage of hNPCs that were edited at the B2M locus, resulting in reduced or lack of B2M expression (B2M-) on the cell surface. The results shown in FIG. 68 illustrate that reducing or depleting CpG motifs within the sequences of the U1a promoter (construct ID 178 and 179), Pol III U6 promoter (construct ID 180 and 181), or bGH poly(A) (construct ID 182) did not significantly decrease editing activity compared to the editing level achieved with the original CpG⁺ AAV construct (construct ID 177). Specifically, CpG⁻ Ula, CpG⁻ U6, or CpG⁻ bGH resulted in ˜80%, ˜94%, or ˜83% editing of the editing level attained with the base CpG⁺ AAV construct. However, reducing or depleting CpG motifs within the UbC promoter sequence (construct ID 184, 185, and 186) substantially diminished editing activity compared to the level seen with the base UbC construct (construct ID 183), highlighting context-dependent effects of CpG depletion on AAV editing activity and underscoring the importance of screening individual CpG-depleted AAV elements to retain potent editing.

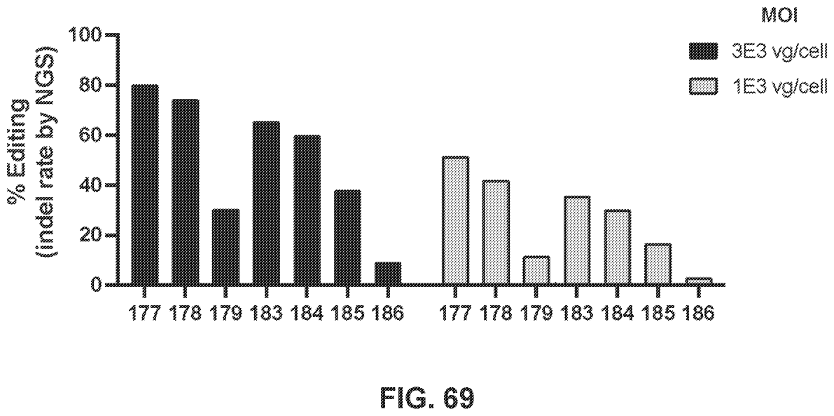

The results presented in bar plot in FIG. 69 illustrate that use of the U1a promoter (construct ID 177) resulted in higher editing at the B2M locus when compared to the editing level after use of the UbC promoter (construct ID 183) at both MOIs. This improvement in editing was recapitulated when comparing the use of their CpG-reduced and CpG-depleted counterparts at both MOIs (compare construct ID 178-179 to construct ID 184-186; FIG. 69). Furthermore, depleting CpGs in either U1a or UbC resulted in reduced editing when compared to the editing observed from using their wild-type (WT) or CpG-reduced counterparts (FIG. 69). Interestingly, depleting CpGs in the U1a promoter nevertheless resulted in relatively higher editing compared to the editing level achieved when depleting CpGs in the UbC promoter (FIG. 69).

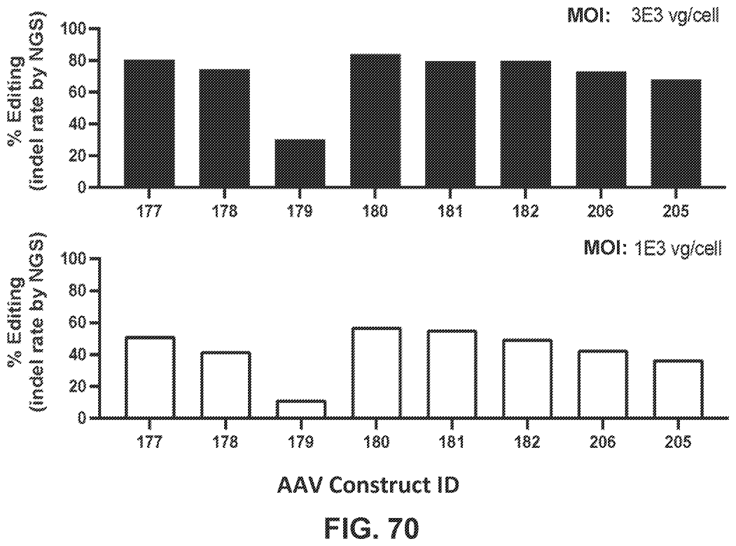

In addition to evaluating the effects of depleting CpGs in different protein promoters (e.g., U1a compared to UbC) on editing mediated by the CasX:gRNA system delivered by AAVs, the effects of depleting CpGs in other elements on editing were analyzed at two MOIs (FIG. 70). Furthermore, individual CpG⁻ elements were combined to generate an AAV genome with substantial CpG depletion, and the consequential effects on editing at the B2M locus were assessed (FIG. 70).

FIG. 70 shows bar plots that illustrate the quantification of percent editing at the B2M locus as detected by NGS seven days post-transduction of AAVs into human iNs at an MOI of 3E3 (FIG. 70, top) or 1E3 (FIG. 70, bottom). Various CpG-reduced or CpG-depleted AAV elements were tested to assess the effects of their use on editing efficiency at the B2M locus as follows: 177 (no CpG depletion); 178 (U1A promoter with reduced CpG); 179: (U1A promoter with CpG depleted); 180 (U6 promoter with reduced CpG); 181(U6 promoter with CpG depleted); 182 (bGH poly(A) with CpG depleted); 206 (U1A promoter with reduced CpG, CasX491 with CpG depleted, bGH with CpG depleted, and U6 promoter with CpG depleted); 205 (U1A promoter with CpG depleted, CasX491 with CpG depleted, bGH with CpG depleted, and U6 promoter with CpG depleted). ITRs are wild-type sequence.

Several key conclusions were determined from these results, illustrated in FIG. 70: 1) use of CpG-depleted U1a promoter resulted in a drastic decrease in editing compared to the editing from using the WT or CpG-reduced Ula, supporting findings observed in the results presented FIG. 69; 2) depleting CpGs in either the bGH-polyA or U6 RNA promoter resulted in similar editing levels as that achieved by their WT counterpart; and 3) combining CpG-depleted or CpG-reduced elements to build a combined AAV genome with substantial CpG reduction could still retain editing activity, as shown in FIG. 70.

Additionally, results from experiments aimed to assess the effects of incorporating CpG-depleted gRNA scaffold constructs into a combined AAV genome with substantial CpG depletion on editing at the B2M locus may reveal that varying levels of editing potency can be achieved when delivered and packaged via AAVs.

These experiments demonstrated that using AAV elements with different levels of CpG depletion can result in varying levels of editing mediated by the CasX:gRNA system when packaged and delivered in vitro via AAVs. The data also demonstrated that depleting CpGs in certain elements could result in similar levels of editing as that achieved when using their WT counterparts. Incorporating CpG-reduced or CpG-depleted elements further expands the inventory of diverse sequences that could be used to build an AAV genome, potentially reducing the risk of recombination during AAV packaging and production.

### Assessment of Use of CpG-Depleted Guide Scaffolds on Editing in a Cell-Based Assay:

Mutations were introduced into the guide scaffold 235 to reduce the CpG content of the DNA sequence coding the guide scaffold. Surprisingly, compared to scaffold 235, all CpG-reduced and CpG-depleted scaffold variants produced higher levels of editing in induced neurons. This was the case with two independent repeats of the experiment (with the results from the first repeat of the experiment shown in FIG. 77A, and the results of the second repeat of the experiment shown in FIGS. 77B-77D), and across multiple MOIs (FIGS. 77B-77D). The enhanced level of editing was surprising because the goal of reducing CpG content was to simply preserve editing activity while reducing immunogenicity. Instead, the mutations enhanced editing activity, rather than merely preserving it.

Notably, scaffold 320 showed a significant increase in potency over scaffold 235. Scaffold 320 includes mutations to only two regions of the scaffold: in the pseudoknot stem and the extended stem (regions 1 and 4). Further, some combinations of mutations produced worse editing than scaffold 320. However, even the CpG-reduced scaffolds that performed worse than scaffold 320, such as scaffolds 331 and 334, performed similarly to or better than scaffold 235.

Based on these results, without wishing to be bound by theory, it is believed that the boost in potency seen in many of the CpG-reduced and CpG-depleted scaffolds is likely caused by one of the mutations present in all CpG-reduced scaffolds (i.e., region 1 and/or 4). Since the mutation to region 4 is not present in the scaffolds with the extended stem loop replacement (i.e., the third mutation to region 3) and these scaffolds show a similar improvement in potency over 235 as 320 did, it is believed that the beneficial effect is likely caused by the mutation in region 1 (pseudoknot stem), which is present in all tested scaffolds. Further experiments are performed to test the effect of the individual mutations in the pseudoknot stem (region 1) and the extended stem (region 4) separately.

Further, the N=1 data as presented in FIG. 77A indicate that all the new scaffolds carrying the mutation in region 2 (scaffold stem) edited at a slightly lower level than their respective counterparts without this mutation. This suggests that mutating this position in the scaffold stem may have a small deleterious effect on editing potency. This is examined in additional experiments.

The results described here demonstrate that introducing mutations that reduced the CpG content of the DNA encoding the guide RNA scaffold resulted in improvements in gene editing relative to guide scaffold 235.

### Example 19: CpG-Depleted AAVs Induce Less TLR9-Mediated Immune Response In Vitro

In the preceding example, CpG-reduced and CpG-depleted AAVs were shown to achieve effective editing at the targeted human B2M locus (as exemplary). Here, experiments are performed to assess the effects of CpG reduction or CpG depletion on the activation of TLR9-mediated immune response in vitro. Individual elements of the AAV genome and their respective CpG-reduced or CpG-depleted versions are subjected to in vitro assessment of immunogenicity to identify the optimal CpG-depleted sequences that reduce undesired TLR9 activation and yield potent editing (as demonstrated in Example 18), before being combined to generate an AAV genome with drastically reduced CpG presence for further evaluation.

### Materials and Methods

AAV plasmid cloning, production of AAV vectors, and titering are performed as described in Example 18.

Use of Human TLR9 Reporter HEK293 Cells (HEK-Blue™ hTLR9) for the In Vitro Immunogenicity Assessment Post-Transduction with CpG-Containing (CpG+) or CpG-Depleted (CpG−) AAVs:

The HEK-Blue™ hTLR9 line (InvivoGen) is derived from HEK293 cells, specifically designed for the study of TLR9-induced NF-κB signaling. These HEK-Blue™ hTLR9 cells overexpress the human TLR9 gene, as well as a SEAP (secreted embryonic alkaline phosphatase) reporter gene under the control of an NF-κB inducible promoter. SEAP levels in the cell culture medium supernatant, which can be quantified using colorimetric assays, report TLR9 activation.

For this experiment, 5,000 HEK-Blue™ hTLR9 cells are plated in each well of a 96-well plate in DMEM medium with 10% FBS and Pen/Strep. The next day, seeded cells were transduced with CpG⁺ or CpG⁻ AAVs expressing the CasX:gRNA system. All viral infection conditions are performed at least in duplicate, with normalized number of viral genomes (vg) among experimental vectors, in a series of three-fold serial dilution of MOI starting with the effective MOI of 1E6 vg/cell. Levels of secreted SEAP in the cell culture medium supernatant are assessed using the HEK-Blue™ Detection kit at 1, 2, 3, and 4 days post-transduction following the manufacturer's instructions.

The experiments using HEK-Blue™ hTLR9 cells to assess TLR9-modulated immune response are expected to show reduced levels of secreted SEAP from cells treated with CpG-AAVs in comparison to levels from cells treated with unmodified CpG⁺ AAVs. Reduced SEAP levels would indicate decreased TLR9-mediated immune activation.

### Example 20: In Vivo Administration of AAV Vectors with or without CpG-Depleted Genomes to Assess the Effects on Inflammatory Cytokine Production and CasX-Mediated Editing

Experiments are performed to assess the effects of administering AAV vectors with or without CpG-depleted genomes in vivo. Briefly, AAV particles expressing the CasX:gRNA system (with or without CpG depletion) are administered into C57BL/6J mice. In these experiments, the combined AAV genome with substantial CpG depletion are used for assessment. After AAV administration, mice are bled at various time points to collect blood samples. Production of inflammatory cytokines such as IL-1β, IL-6, IL-12, and TNF-α is measured using ELISA and an assay that assesses transgene-specific T cell populations generated against the SIINFEKL (SEQ ID NO: 9589) peptide.

### Materials and Methods

### Generation of CpG-Depleted AAV Plasmids:

To assess the generation of transgene-specific T cells, a sequence encoding a SIINFEKL peptide (SEQ ID NO: 9589) is cloned into an AAV transgene plasmid on the 5′- and 3′-terminus of the encoded CasX protein, along with a gRNA with a ROSA26-targeting spacer. The SIINFEKL peptide (SEQ ID NO: 9589) is an ovalbumin-derived peptide that is well-characterized and has widely available reagents to probe for T cells specific for this peptide epitope.

Production of AAV vectors and determination of viral genome titer is performed as described earlier in Example 17.

### Measurement of Inflammatory Cytokines to Assess Humoral Immune Activation:

˜1E12 vg AAVs are injected intravenously or intraperitoneally into C57BL/6J mice. Blood is drawn daily from the tail vein or saphenous vein for seven days after AAV injection. Collected blood serum is assessed for the levels of inflammatory cytokines, such as IL-1β, IL-6, IL-12, and TNF-α using commercially available ELISA kits according to the manufacturer's recommendations for murine blood samples (Abcam). Briefly, 50 μL of standard, control buffer, and sample is loaded to the wells of an ELISA plate, pre-coated with a specific antibody to IL-1β, IL-6, IL-12, or TNF-α, incubated at room temperature (RT) for two hours, washed, and incubated with horseradish peroxidase enzyme (HRP) for two hours at RT, followed by additional washes. Wells are treated with TMB ELISA substrate and incubated for 30 minutes at RT in the dark, followed by quenching with H₂SO₄. Absorbance is measured at 450 nm using a TECAN spectrophotometer with wavelength correction at 570 nm.

### Assessment of Transgene-Specific T Cell Populations:

Ten days after intravenous injection with AAVs, the spleen is collected from mice, and T cells are isolated using the EasySep™ Mouse T Cell Isolation kit. Isolated T cells are incubated with the following: FITC mouse anti-human CD4 antibody (BD Biosciences), APC mouse anti-human CD8 antibody (BD Biosciences), and BV421 ovalbumin SIINFEKL MHC tetramer (Tetramer Shop). The percentage of CD4+ and CD8+ T cells specific to the SIINFEKL MHC tetramer is quantified using flow cytometry. FITC, APC, and BV421 are excited by the 488 nm, 561 nm, and 405 nm lasers and signal are quantified using suitable filter sets.

### Quantification of AAV-Mediated Genome Editing at the ROSA26 Locus:

To demonstrate that CpG⁻ AAVs exhibit enhanced CasX editing activity relative to CpG⁺ AAVs in vivo, ˜1E12 AAV particles containing CasX protein 491 with gRNA targeting the ROSA26 locus are administered intravenously via the facial vein of C57BL/6J mice. Four weeks post-injection, mice are euthanized, and the liver and/or muscle tissue are harvested for gDNA extraction using the Zymo Quick DNA/RNA miniprep Kit following the manufacturer's instructions. Target amplicons are amplified from 200 ng of extracted gDNA with a set of primers targeting the mouse ROSA26 locus of interest and processed for NGS as described earlier in Example 18.

In vivo experiments measuring serum inflammatory cytokine levels are expected to show that CpG-depleted AAVs would significantly dampen production of inflammatory cytokines, such as IL-1β, IL-6, IL-12, and TNF-α, thereby reducing immunogenicity and toxicity. In addition, CpG-depleted AAVs are likely to cause less TLR9 activation leading to reduced expansion of T cells against the SIINFEKL peptide (SEQ ID NO: 9589)fused to CasX. Therefore, injections with CpG-depleted AAVs are expected to yield decreased levels of SIINFEKL-specific CD4+ and CD8+ T cells compared to levels from AAV constructs containing CpG elements.

Since CpG-depleted AAVs are likely to cause less humoral immune activation and non-specific inflammation, as well as less T-cell mediated immunity, titers of CasX-reactive antibodies are also expected to be reduced (i.e., lower ELISA signal quantifying CasX antibodies are anticipated).

Finally, editing capabilities of CpG-depleted AAVs are assessed by harvesting muscle and/or liver tissue for genomic DNA extraction and subjected to NGS to determine editing levels at the ROSA26 locus. Enhanced CasX editing activity at the ROSA26 locus is anticipated with CpG-depleted AAVs, given their expected likelihood to elicit less humoral immune response in vivo.

### Example 21: Use of Muscle-Specific Promoters to Drive CasX Expression Results in Editing Activity in Muscle Cells and Tissue when the CasX:gRNA System is Expressed from a Transfected AAV Plasmid In Vitro or Packaged and Delivered Via AAVs In Vitro and In Vivo

Experiments were performed to demonstrate that use of muscle-specific promoters to drive CasX expression in an AAV vector results in higher and more selective editing activity in muscle cells than in non-muscle cell types, when the CasX:gRNA system is expressed from an AAV plasmid transfected in vitro. Experiments were also performed to demonstrate that use of muscle-specific promoters to drive CasX expression results in editing at a target locus in muscle cells when the CasX:gRNA system is packaged and delivered via AAVs in vitro and in vivo.

### Materials and Methods

CasX variant 491 and guide scaffold variant 235 were used in these experiments. AAV construct cloning was performed as similarly described in Example 1. Briefly, AAV constructs containing a muscle-specific promoter driving CasX expression and a Pol III U6 promoter driving the expression of gRNA scaffold 235 and a ROSA26-targeting spacer (spacer 35.2; refer to Table 41 for sequences) were generated using standard molecular cloning techniques. Sequence-validated plasmid constructs were midi-prepped for subsequent nucleofection.

TABLE 41
Sequences of AAV constructs used in this example for testing muscle-specific
promoters.

| | | DNA sequence | Length of |
|---|---|---|---|
| Construct ID | Component Name | or SEQ ID NO | Component (bp) |
| 215 through 220 | 5′ ITR | 3683 | 130 |
| | buffer sequence | 3684 | 23 |
| 215, 220 | UbC promoter | 3720 | 400 |
| 216 | CK8e promoter | 3775 | 450 |
| 217 | MHCK7 promoter | 3777 | 776 |
| 218 | Desmin promoter | 3774 | 724 |
| 219 | MHCK promoter | 3776 | 742 |
| 215 through 220 | buffer sequence | 9302 | 18 |
| | Kozak | GCCACC | 6 |
| | start codon | ATGGCC | 6 |
| | c-MYC NLS | 9290 | 27 |
| | linker | 9306 | 6 |
| | CasX 491 | 9291 | 2931 |
| | linker | GGCTCC | 6 |
| | c-MYC NLS | 9292 | 27 |
| | stop codon | TAA | 3 |
| | buffer sequence | 3695 | 30 |
| | bGH poly(A) | 3696 | 209 |
| | buffer sequence | GGTACCGT | 8 |
| | U6 promoter | 3698 | 242 |
| | buffer sequence | GAAACACC | 8 |
| | Scaffold 235 | 3631 | 89 |
| 215 through 219 | Spacer 35.2 | 9317 | 20 |
| 220 | Spacer NT | 9314 | 18 |
| 215 through 220 | buffer sequence | 3700 | 17 |
| | 3′ ITR | 3701 | 141 |

*Components are listed in a 5′ to 3′ order within the constructs

Plasmid Nucleofection into Mouse NPCs and Mouse C2C12 Myoblasts:

Briefly, 1 g of individual AAV plasmids (Table 41) expressing the CasX under the control of different muscle promoters were nucleofected into mouse muscle C2C12 myoblast cells, as well as neuronal NPCs for each experimental condition using methods as described in Example 1. Full media replacement was performed 48 hours post-nucleofections. Five days post-nucleofection, treated cells were harvested for gDNA extraction using the Zymo Quick DNA™ 96 Kit following the manufacturer's instructions. Target amplicons were then amplified from 200 ng of extracted gDNA with a set of primers targeting the mouse ROSA26 locus and processed as described earlier in Example 18 for editing assessment by NGS. As experimental controls, AAV plasmid constructs encoding the following were also tested: 1) UbC promoter driving CasX expression with gRNA containing spacer 35.2; and 2) UbC promoter driving CasX expression with a non-targeting gRNA. A ‘no treatment’ control was also included as an experimental control.

AAV production and AAV titering were performed as described in Example 1.

### AAV Transduction of C2C12 Myoblasts and Myotubes:

AAVs were used to transduce two differentiated states of C2C12 cells—myoblasts and myotubes.

To determine the level of CasX-mediated editing in myoblasts, ˜5,000 C2C12 myoblasts were plated and transduced the next day with AAVs encoding the various CasX:gRNA systems (Table 41) at varying MOIs. Five days following transduction, cells were harvested for gDNA extraction for editing analysis at the ROSA26 locus as described above.

To determine the level of CasX-mediated editing in myotubes, ˜10,000 C2C12 myoblasts were plated and cultured in differentiation media for seven days to induce differentiation into myotubes. After myotube formation, cells were transduced with AAVs encoding the various CasX:gRNA systems (Table 41) at varying MOIs. Five days following transduction, cells were harvested for gDNA extraction for editing analysis at the ROSA26 locus as described above.

### In Vivo Administration of AAVs and Tissue Processing:

˜8E11 AAV viral particles encoding the various CasX:gRNA systems (Table 41) were administered retro-orbitally in C57BL/6J adults. Naïve, untreated mice served as experimental controls. Mice were euthanized at four weeks post-injection. Various tissues were harvested for gDNA extraction using the Zymo Quick DNA/RNA™ miniprep Kit following the manufacturer's instructions. Tissues harvested were skeletal muscles (i.e., tibialis anterior (TA), gastrocnemius (GA), quadriceps (Quad), heart, and diaphragm (DIA)) and non-muscle organs (i.e., liver and lung). Target amplicons were amplified from 200 ng of extracted gDNA with a set of primers targeting the mouse ROSA26 locus and processed as described in Example 18 for editing assessment by NGS. The number of AAV viral genomes (vg) per diploid genome (dg) was determined in the harvested gDNA samples by droplet digital PCR (ddPCR) using the Bio-Rad QX200 Droplet Digital PCR instrument according to standard methods and following the manufacturer's guidelines (see additional detail in Example 28). The vg/dg analysis is an indication of the amount of AAV viral particles delivered into that specific tissue.

### Results

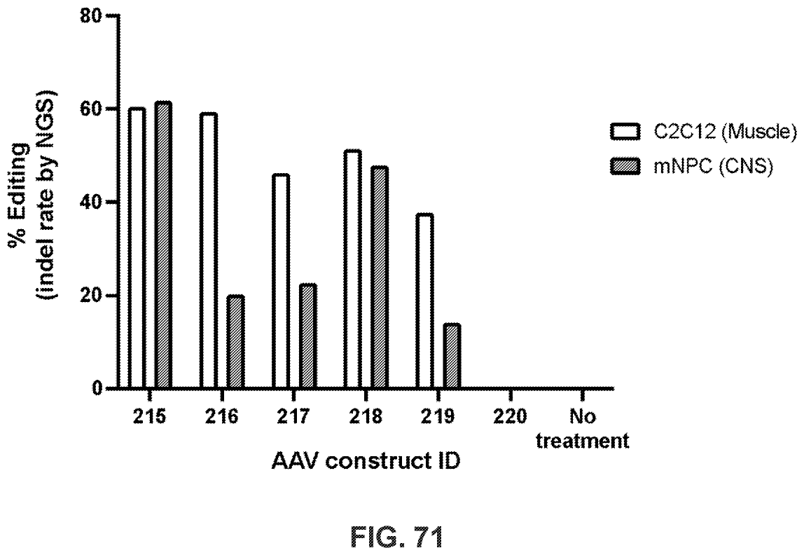

AAV plasmids containing constructs encoding for muscle-specific promoters used to drive CasX expression were nucleofected into C2C12 myoblasts and mouse NPCs to assess the level of specificity of the editing activity in muscle cells compared to neuroprogenitor cells. FIG. 71 shows the quantification of editing measured as indel rate detected by NGS at the mouse ROSA26 locus in C2C12 cells and mouse NPCs for the indicated AAV plasmids. Of the four muscle-specific promoters assessed, use of promoters CK8e (construct ID 216), MHCK7 (construct ID 217), and MHCK (construct ID 219) resulted in higher CasX-mediated editing activity in C2C12 muscle cells compared to that seen in mouse NPCs. Specifically, use of the CK8e promoter resulted in ˜60% editing at the ROSA26 locus in myoblasts but ˜20% editing in mNPCs, indicating that use of the CK8e promoter would result in more selective expression and higher activity in muscle cell types than neuronal cell types (FIG. 71). Meanwhile, use of the Desmin promoter (construct ID 218) resulted in similar editing levels in both C2C12 cells and mouse NPCs, suggesting minimal tissue-specificity effects when utilizing the Desmin promoter. As anticipated, use of the ubiquitous UbC promoter resulted in similar levels of editing activity in both cell types, while no editing was observed with use of the non-targeting spacer or in the ‘no treatment’ control (FIG. 71).

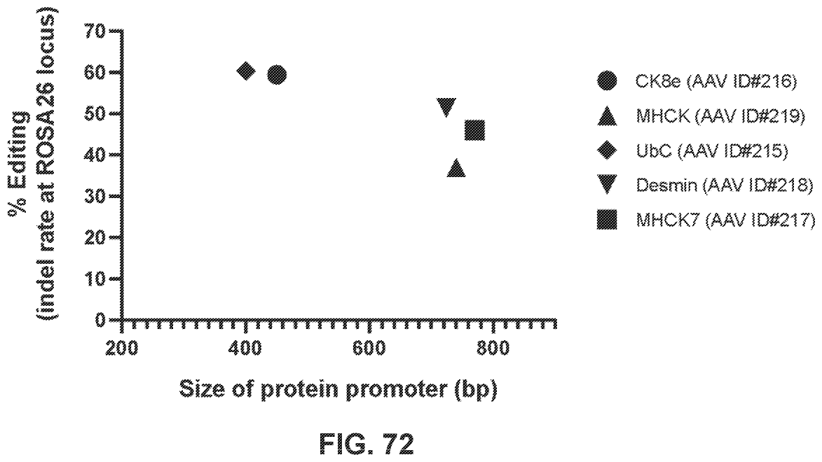

Furthermore, the percent editing at the ROSA26 locus was plotted against the size of the muscle-specific protein promoter, with the results presented in FIG. 72. Of the four tested muscle-specific promoters, the CK8e promoter (construct ID 216) has a similar size of ˜400 bp as that of the UbC promoter and demonstrated a similar level of editing activity.

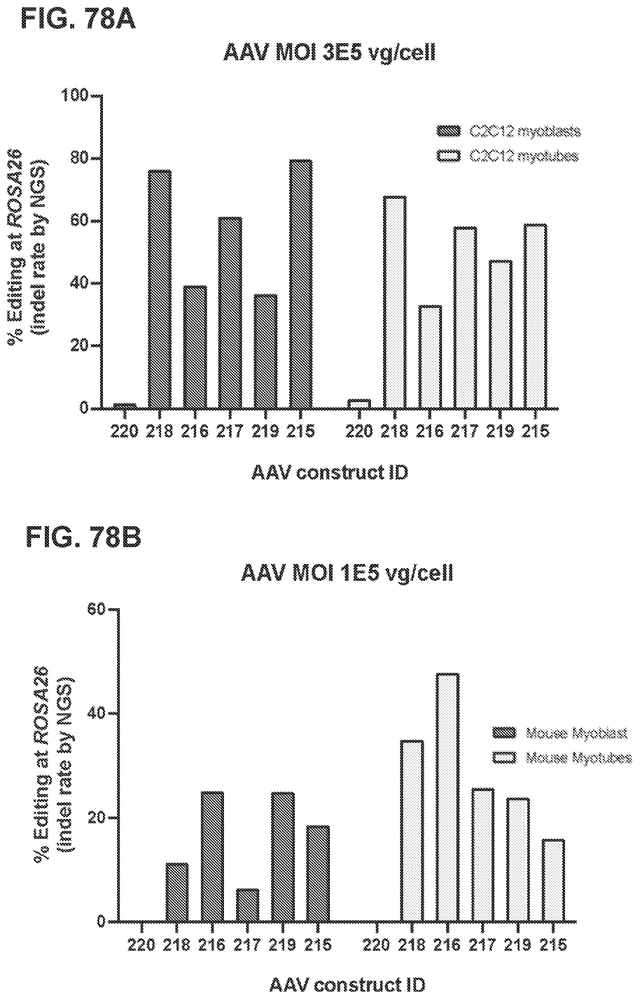

AAVs encoding the CasX:gRNA system, in which muscle-specific promoters were used to drive CasX expression, were used to transduce C2C12 myoblasts and myotubes to assess the level of editing activity in muscle cells at the ROSA26 locus, and the editing results are illustrated in FIGS. 78A and 78B for MOI of 3E5 vg/cell and 1E5 vg/cell respectively. The data demonstrate that use of all four muscle-specific promoters, Desmin, CK8e, MHCK7, and MHCK, were able to induce editing at the ROSA26 locus in both types of muscle cells, albeit at variable levels, when packaged and delivered within AAVs.

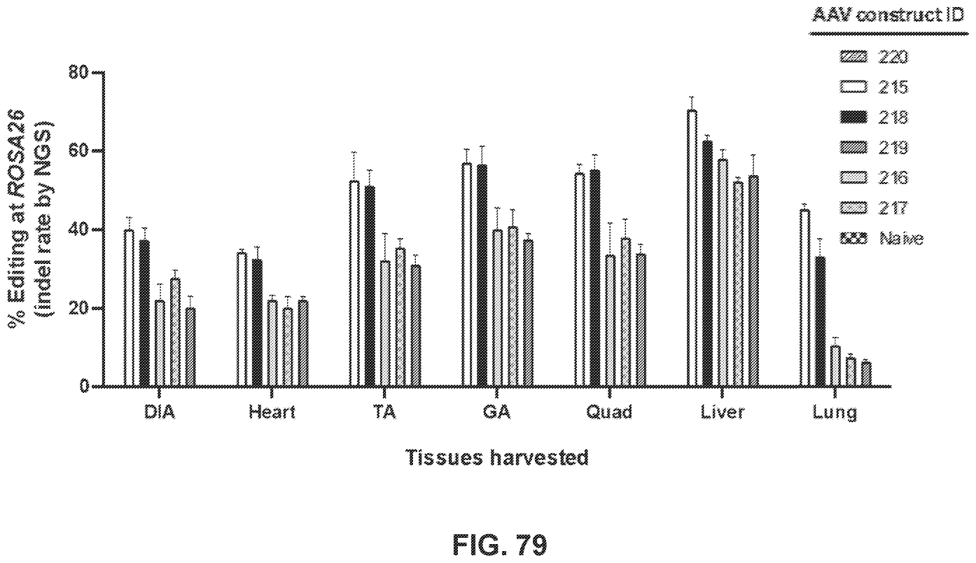

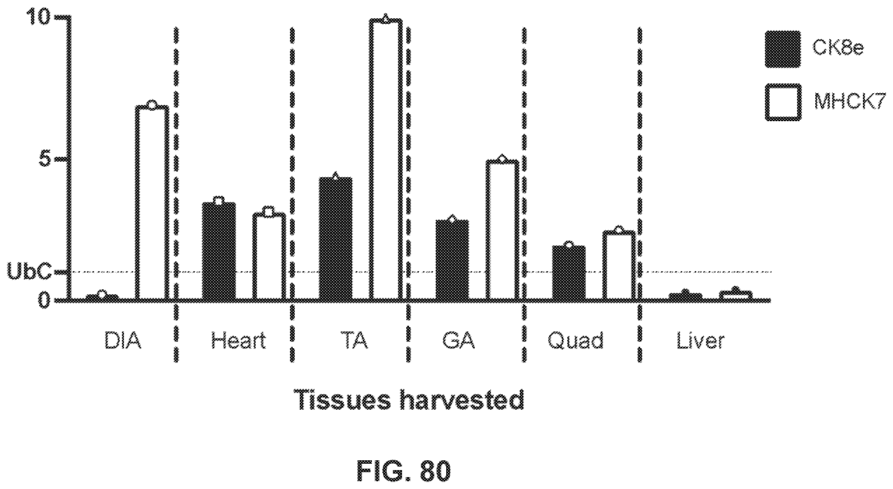

An initial proof-of-concept experiment assessing use of the four different muscle-specific promoters was performed in vivo. AAVs containing CasX protein 491, driven by the muscle-specific promoters or the UbC promoter, and guide scaffold 235 with the ROSA26-targeting spacer were delivered in vivo. Both muscle and non-muscle organs were harvested for editing and vg/dg analyses, depicted in bar graphs in FIG. 79 and FIG. 80 respectively. The data in FIG. 79 show that use of AAVs containing muscle-specific promoters driving CasX expression and a ROSA26-targeting gRNA resulted in varying levels of editing activity across all the harvested tissues. In the muscle tissues (DIA, heart, TA, GA, and Quad), AAVs with the muscle-specific promoters demonstrated lower editing activity compared to that when using the UbC promoter to drive CasX expression. However, in the lung, selective editing activity was detected, such that use of muscle-specific promoters resulted in substantially lower editing activity in the lung compared to that of the UbC promoter, suggesting de-targeting of editing activity in the lung (FIG. 79). The results also show that systemic administration of AAVs using either UbC or muscle-specific promoters induced high editing levels in the liver.

AAV biodistribution was evaluated by quantifying AAV viral particles delivered for a specific tissue using a vg/dg analysis. The vg/dg analysis revealed that similar biodistribution levels were achieved for AAVs containing a muscle-specific promoter or the UbC promoter within a particular tissue (data not shown). Further analysis was performed to determine the relative CasX expression (normalized by vg/dg) driven by muscle-specific promoters CK8e or MHC7 compared to that driven by UbC, and the results are illustrated in FIG. 80. The data demonstrate that after normalizing for delivery to each tissue, use of either muscle-specific promoter CK8e or MHCK7 resulted in higher CasX expression in the muscle tissues relative to the UbC promoter overall, whereas CasX expression in the liver was similar among the promoters compared (FIG. 80). These findings support the significance of using tissue-specific promoters to drive CasX expression within the target tissue to induce editing.

The results demonstrate that muscle-specific promoters can be used to drive CasX expression and induce higher editing activity in muscle cell types than in non-muscle cell types when delivered via nucleofection. The data also show that AAVs produced from these AAV plasmids containing muscle-specific promoters were able to induce CasX expression and editing activity in muscle cells in vitro and in vivo when delivered via transduction. The findings also indicate the tissue specificity of using muscle-specific promoters to drive CasX expression compared to use of ubiquitous promoter like UbC.

### Example 22: Use of Muscle-Specific Regulatory Elements to Generate AAV Constructs to Produce AAVs that would Transduce and Express CasX More Selectively in Muscle Cells and Tissue

Experiments are performed to demonstrate that incorporation of muscle-specific regulatory elements, e.g., promoters and enhancers, into AAV plasmids used for AAV production, results in more selective expression of CasX and higher editing activity in muscle cell types than in non-muscle cell types when the CasX:gRNA system is delivered by AAVs.

### Materials and Methods

CasX variant 491, 515, 593, 668, 672, 676, or 812 are used for the experiments described herein. AAV construct cloning, AAV production, and AAV titering are performed as described in Example 1. Various muscle-specific regulatory elements, e.g., promoters (Table 42) and enhancers (Table 43), are individually cloned into AAV plasmids harboring sequences encoding for a CasX protein and a gRNA with scaffold 235 and an AAVS1-targeting spacer. The resulting AAV plasmids are used for AAV production and transduction of human skeletal muscle cells (hSKMCs) to determine editing levels at the AAVS1 locus.

TABLE 42
Sequences of muscle-specific promoters.

| SEQ ID NO | Promoter | Promoter length (bp) |
|---|---|---|
| 3773 | SP-301 | 579 |
| 3774 | Desmin | 724 |
| 3775 | CK8e | 450 |
| 3776 | MHCK | 742 |
| 3777 | MHCK7 | 776 |
| 3778 | SpC5-12 | 358 |

TABLE 43
Sequences of muscle-specific enhancers.

| SEQ ID NO | Enhancer | Enhancer length (bp) |
|---|---|---|
| 3779 | Muscle Enhancer 1 | 495 |
| 3780 | Muscle Enhancer 2 | 344 |
| 3781 | Muscle Enhancer 3 | 429 |
| 3782 | Muscle Enhancer 4 | 434 |
| 3783 | Muscle Enhancer 5 | 171 |
| 3784 | Muscle Enhancer 6 | 51 |
| 3785 | Muscle Enhancer 7 | 60 |
| 3786 | Muscle Enhancer 8 | 41 |
| 3787 | Muscle Enhancer 9 | 120 |
| 3788 | Muscle Enhancer 10 | 474 |
| 3789 | Muscle Enhancer 11 | 519 |
| 3790 | Muscle Enhancer 12 | 372 |
| 3791 | MyoD Enhancer | 256 |
| 3792 | Cardiac Muscle Enhancer 1 | 206 |
| 3793 | Cardiac Muscle Enhancer 2 | 255 |
| 3794 | Cardiac Muscle Enhancer 3 | 277 |
| 3795 | Cardiac Muscle Enhancer 4 | 660 |
| 3796 | Myoblast Muscle Enhancer 1 | 221 |
| 3797 | Myoblast Muscle Enhancer 2 | 310 |
| 3798 | Myoblast Muscle Enhancer 3 | 218 |
| 3799 | Myoblast Muscle Enhancer 4 | 353 |
| 3800 | Myoblast Muscle Enhancer 5 | 50 |
| 3801 | Skeletal Muscle Enhancer 1 | 395 |
| 3802 | Skeletal Muscle Enhancer 2 | 382 |
| 3803 | Skeletal Muscle Enhancer 3 | 135 |
| 3804 | Skeletal Muscle Enhancer 4 | 326 |
| 3805 | Skeletal Muscle Enhancer 5 | 273 |
| 3806 | Skeletal Muscle Enhancer 6 | 148 |
| 3807 | Skeletal Muscle Enhancer 7 | 80 |
| 3808 | Skeletal Muscle Enhancer 8 | 437 |
| 3809 | Skeletal Muscle Enhancer 9 | 297 |

### AAV Transduction In Vitro:

AAVs are used to transduce two differentiated states of hSKMCs—myoblasts versus myotubes.

500,000 primary hSKMC cells (ATCC, PCS-950-010) are plated per 2-4×15 cm dishes in growth media (DMEM/F-12, 20% FBS, 1% PenStrep, 2.5 ng/mL b-FGF). Once cells reach 7000 confluency, cells are lifted and re-seeded in a 96-well plate at 5,000-10,000 cells per well in differentiation media (DMVEM, 200 horse serum, 1% PenStrep).

To determine level of CasX-mediated editing in myoblasts, hSKMCs are transduced with AAVs 4-6 hours after re-seeding in differentiation media at multiple MOIs. Five days following transduction, cells are harvested for gDNA extraction for editing analysis at the AAVS1 locus by NGS. Briefly, target amplicons are amplified from 200 ng of extracted gDNA with a set of primers targeting the human AAVS1 locus and processed for NGS as described in Example 18.

To determine the level of CasX-mediated editing in myotubes, re-seeded hSKMCs into differentiation media are cultured in differentiation media for an additional 7-10 days to promote differentiation into myotubes. After myotube formation, cells are transduced with AAVs at multiple MOIs. Five days following transduction, cells are harvested for editing assessment at the AAVS1 locus by NGS as described above.

As a comparison to assess muscle-cell specificity of the produced AAVs, non-muscle cells such as HepG2 hepatocytes or human NPCs are also transduced with AAVs produced from the same AAV plasmids containing the muscle-specific regulatory elements described herein.

In addition, assessing the incorporation of muscle-specific regulatory elements within an AAV transgene to selectively express CasX in muscle-specific cell types in vivo are also investigated. These methods for these in vivo experiments are further described in Example 23.

The results of these experiments are expected to demonstrate that AAVs produced from AAV plasmids containing constructs incorporating muscle-specific regulatory elements (promoter and/or enhancer, see Tables 42 and 43) to drive CasX expression, demonstrate higher editing activity in muscle-specific cell lines compared to non-muscle cell types.

### Example 23: Use of Muscle-Specific AAV Serotypes to Increase Muscle-Specific Cellular and Tissue Tropism to Enhance CasX-Mediated Editing In Vivo

Experiments are performed to demonstrate that use of muscle-specific AAV serotypes may improve specific cellular and tissue tropism and, therefore, enhance delivery and potency of AAVs in the target muscle cells with minimal editing in off-target cell types in vivo.

### Materials and Methods

AAV plasmid cloning and AAV production and titering are performed using similar methods described in Example 1. Specifically, the sequences encoding the AAV VP1 serotypes and variants listed in Table 44 are cloned into relevant pRep/Cap plasmids for use in AAV production.

TABLE 44
Sequences of AAV serotypes to be assessed in vivo.

| | Amino acid sequence |
|---|---|
| AAV serotype | (SEQ ID NO) |
| AAV6 | 3810 |
| Rh74 | 3811 |
| RhM4-1 | 3812 |
| AAV9 | 3813 |
| MyoAAV 1A1 | 3814 |
| MyoAAV 1A2 | 3815 |
| MyoAAV 2A | 3816 |

### In Vivo Administration of AAVs and Tissue Processing:

A dose response experiment is performed, where ˜1E9 to 1E12 AAV viral particles containing CasX protein 491, 515, 672, or 676 and guide scaffold variant 235 with spacer 35.2 targeting the safe harbor ROSA26 locus are administered retro-orbitally in C57BL/6J adults. Naïve, untreated mice serve as experimental controls. Mice are euthanized at different time points, up to four weeks post-injection. Various tissues, including skeletal muscles (e.g., tibialis anterior, gastrocnemius, soleus, quadriceps, heart, and diaphragm) and other organs (liver, spleen, lung etc.) are harvested for gDNA extraction using the Zymo Quick DNA/RNA miniprep Kit following the manufacturer's instructions. Target amplicons are then amplified from 200 ng of extracted gDNA with a set of primers targeting the mouse ROSA26 locus and processed as described earlier in Example 18 for editing assessment by NGS.

Results from the experiments are expected to show that AAVs containing CasX protein and guide scaffold 235 with the ROSA26-targeting spacer are able to edit the target ROSA26 locus in various muscle tissues. Furthermore, it is expected that higher editing activity is detected in muscle tissues compared to that detected in other tissues, such as the liver or spleen, which would indicate the ability to increase muscle-specific tissue tropism in vivo by incorporating constructs encoding for muscle-specific AAV serotypes into the pRep/Cap plasmid.

### Example 24: Small Class 2, Type V CRISPR Proteins can Edit the Genome when Expressed from an AAV Episome In Vitro

Experiments are conducted to demonstrate that small Class 2, Type V CRISPR proteins, such as CasX, are able edit a genome when expressed from an AAV plasmid or an AAV vector in vitro.

### Materials and Methods

The AAV transgene is conceptually broken up between ITRs into different parts, consisting of the therapeutic cargo and accessory elements relevant to expression of the therapeutic cargo in mammalian cells. AAV vectorology consists of identifying the relevant parts and subsequently designing, building, and testing vectors in both plasmid and AAV vector form in mammalian cells. A schematic of one configuration of its components is shown in FIG. 1.

For the experiments in this example, constructs encoding for CasX variants 515, 593, 668, 672, 676, or 812 with gRNA scaffold variant 235 are used to generate AAV plasmids for AAV production.

AAV vector cloning and quality control are performed as described in Example 1.

Method for plasmid nucleofection are performed as described in Example 1.

AAV production and titer determination are performed as described in Example 1.

AAV transduction of mNPCs and subsequent FACS analysis for tdTomato+ cell quantification are performed as described in Example 1.

The results are expected to demonstrate that small CRISPR proteins, such as CasX, and targeted gRNAs are able to edit the genome when expressed from an AAV transgene plasmid or episome in vitro. For the experiments described in this example, the results are expected to show that CasX variant proteins complexed with a gRNA containing scaffold 235 and a tdTomato-targeting spacer are able to edit the target STOP cassette in mNPCs as measured by FACs.

### Example 25: Packaging of Small Class 2, Type V CRISPR Systems within an AAV Vector

Experiments are conducted to demonstrate that systems of small Class 2, Type V CRISPR proteins, such as CasX, and gRNA can be encoded and efficiently packaged within a single AAV vector.

### Materials and Methods

For this experiment, AAV vectors are generated with transgenes packaging CasX variant 515, 668, 672, or 676, with gRNA scaffold variant 235 and spacer 12.7 using the methods for AAV production, purification and characterization, as described in Example 24. For characterization, AAV viral genomes are titered by qPCR, and the empty-full ratio are quantified using scanning transmission electron microscopy (STEM). AAVs are negatively stained with 1% uranyl acetate and visualized. Empty particles are identified by the presence of a dark electron dense circle at the center of the capsid.

The results from these experiments are expected to demonstrate that the CRISPR system (CasX variant proteins and gRNA) can be efficiently packaged within a single AAV vector. qPCR results reveal high AAV genome titers, and STEM micrographs are expected to show that a vast majority of the AAV particles contain viral genomes.

### Example 26: Demonstration of In Vivo Editing with Small Class 2, Type V CRISPR Proteins Expressed from an AAV Episome

Experiments are conducted to demonstrate that small Class 2, Type V CRISPR proteins, such as CasX, can edit the genome when expressed from an AAV episome in vivo.

### Materials and Methods

For this experiment, AAV vectors are generated using the methods for AAV production, purification and characterization, as described in Example 24.

### In Vivo Administration of AAVs and Tissue Processing:

Ai9 neonate mice are injected with AAVs with a transgene encoding CasX variant 515, 593, 668, 672, 676, or 812, and gRNA scaffold variant 235 with spacer 12.7. Briefly, mice are cryo-anesthetized, and ˜1E11 viral genomes of AAV particles are unilaterally injected into the intracerebroventricular (ICV) space. One month after ICV injections, animals are terminally anesthetized with an intraperitoneal injection of ketamine/xylazine and perfused transcardially with saline and fixative (4% PFA). Brains are dissected and further post-fixed in 4% PFA, followed by infiltration with 30% sucrose solution, and OCT embedding. OCT-embedded brains are coronally sectioned using a cryostat. Sections are mounted on slides, counterstained with DAPI to label cell nuclei, cover-slipped and imaged on a fluorescence microscope. Images are processed using the ImageJ software and editing levels are quantified by counting the number of tdTomato+ cells as a percentage of DAPI-labeled nuclei.

In a subsequent experiment to assess editing in peripheral tissues, particularly in the liver and heart, P0-P1 pups from Ai9 mice are cryo-anesthetized and intravenously injected with ˜1E12 viral genomes (vg) of the same AAV construct. One month post-AAV administration, animals are terminally anesthetized, and heart and liver tissues are necropsied and processed as described above.

The results of these experiments are expected to demonstrate that AAVs encoding small CRISPR proteins, such as CasX, and a targeting gRNA can be distributed within the tissues, when delivered either locally (brain) or systemically. Given the use of the Ai9 mouse model, the results are expected to show tdTomato fluorescence throughout the target tissue, an indication that the AAV delivery modality would be able to achieve sufficient biodistribution. The results are also expected to show that the CasX:gRNA system can edit the target genome (here, the tdTomato locus) when expressed from single AAV episomes in vivo.

### Example 27: Small CRISPR Protein Potency is Enhanced by AAV Vector Protein Promoter Choice

Experiments are conducted to demonstrate that small CRISPR protein expression, such as CasX, can be enhanced by utilizing different promoters in an AAV construct for the encoded protein. Cargo space in the AAV transgene can be maximized with the use of short promoters in combination with CasX. Additionally, experiments are conducted to demonstrate that expression can be enhanced with the use of promoters that would otherwise be too long to be efficiently packaged within an AAV vector, if combined with larger CRISPR proteins such as Cas9. The use of long, cell-type-specific promoters to enhance small CRISPR proteins is an advantage to the AAV system described herein, and not possible in traditional CRISPR systems due to the size of other CRISPR proteins.

### Materials and Methods

Cloning and QC are conducted as described in Example 24. Promoter variants (listed in Table 7 in example 4) are cloned upstream of the CasX gene in an AAV-cis plasmid. The sequences of additional components of the AAV constructs, with the exception of sequences encoding the CasX (Table 26) and the one or more gRNA (Tables 23 and 24), are listed in Table 45.

### Method for Plasmid Nucleofection:

Immortalized neural progenitor cells are nucleofected as described in Example 24.

AAV viral production and QC, and AAV transduction and editing level assessment in mNPTC-tdT cells by FACS are conducted as described in Example 24.

The results of these experiments are expected to demonstrate that several different promoters used to drive CasX expression, when delivered by nucleofection as an AAV transgene plasmid or by transduction of AAV vectors, are able to induce editing of the target STOP cassette in mNPCs. It is possible that use of certain promoters results in higher editing than use of other promoters. These data are expected to demonstrate that expression of small CRISPR proteins, such as CasX, can be enhanced by utilizing long promoters that would otherwise be unusable with traditional CRISPR proteins due to the size constraints of the AAV genome. Furthermore, combining short promoters with small CRISPR proteins, such as CasX, is expected to allow for significant reductions in the AAV transgene cargo capacity without compromising expression efficiency. This conservation of space allows for the inclusion of additional accessory elements, such as enhancers and regulatory elements in the transgene, which would enable increased editing potential.

TABLE 45
AAV transgene constructs and component sequences*
*Table lists component sequences except for sequences encoding nuclease, guide RNA, and
linking peptides

| | | SEQ | |
|---|---|---|---|
| Component | Name | ID NO | Constructs |
| 5′ ITR | AAV2 ITR (alternate) | 3817 | 1-174, 177-186, 188-198, 207-220 |
| | CpG-depleted 5′ITR | 3749 | none |
| Enhancer + | CMV | 4048 | 1-3, 7, 24-33, 44-52, 103-117, 211-214 |
| core | N/A | 3562 | 1-3, 7, 24-33, 44-52, 64-71, 103-117, 156 |
| promoter | Syn 1 | 3818 | 65 |
| | NPC5 | 3819 | 66 |
| | NPC7 | 3820 | 67 |
| | NPC127 | 3821 | 68 |
| | NPC190 | 3822 | 69 |
| | NPC249 | 3823 | 70 |
| | NPC286 | 3824 | 71 |
| Enhancer | Muscle Enhancer 1 | 3825 | none |
| | Muscle Enhancer 2 | 3826 | none |
| | Muscle Enhancer 3 | 3827 | none |
| | Muscle Enhancer 4 | 3828 | none |
| | Muscle Enhancer 5 | 3829 | none |
| | Muscle Enhancer 6 | 3830 | none |
| | Muscle Enhancer 7 | 3831 | none |
| | Muscle Enhancer 8 | 3832 | none |
| | Muscle Enhancer 9 | 3833 | none |
| | Muscle Enhancer 10 | 3834 | none |
| | Muscle Enhancer 11 | 3835 | none |
| | Muscle Enhancer 12 | 3836 | none |
| | MyoD Enhancer | 3837 | none |
| | Cardiac Muscle Enhancer | 3838 | none |
| | 1 | | |
| | Cardiac Muscle Enhancer | 3839 | none |
| | 2 | | |
| | Cardiac Muscle Enhancer | 3840 | none |
| | 3 | | |
| | Cardiac Muscle Enhancer | 3841 | none |
| | 4 | | |
| | Myoblast Muscle | 3842 | none |
| | Enhancer 1 | | |
| | Myoblast Muscle | 3843 | none |
| | Enhancer 2 | | |
| | Myoblast Muscle | 3844 | none |
| | Enhancer 3 | | |
| | Myoblast Muscle | 3845 | none |
| | Enhancer 4 | | |
| | Myoblast Muscle | 3846 | none |
| | Enhancer 5 | | |
| | Skeletal Muscle Enhancer | 3847 | none |
| | 1 | | |
| | Skeletal Muscle Enhancer | 3848 | none |
| | 2 | | |
| | Skeletal Muscle Enhancer | 3849 | none |
| | 3 | | |
| | Skeletal Muscle Enhancer | 3850 | none |
| | 4 | | |
| | Skeletal Muscle Enhancer | 3851 | none |
| | 5 | | |
| | Skeletal Muscle Enhancer | 3852 | none |
| | 6 | | |
| | Skeletal Muscle Enhancer | 3853 | none |
| | 7 | | |
| | Skeletal Muscle Enhancer | 3854 | none |
| | 8 | | |
| | Skeletal Muscle Enhancer | 3855 | none |
| | 9 | | |
| Protein | CMV | 3856 | 1-3, 7, 24-33, 44-52, 103-117, 211-214 |
| promoter | UbC | 3857 | 4, 34-37, 53, 78, 79-102, 119-155, 157- |
| | | | 174, 183, 188-190, 207-210, 215, 220 |
| | EFS | 3858 | 5, 38-40 |
| | CMV-s | 3859 | 6, 41-43 |
| | CMVd1 | 3860 | 8 |
| | CMVd2 | 3861 | 9 |
| | miniCMV | 3862 | 10 |
| | HSVTK | 3863 | 11 |
| | miniTK | 3864 | 12 |
| | miniIL2 | 3865 | 13 |
| | GRP94 | 3866 | 14 |
| | Supercore 1 | 3867 | 15 |
| | Supercore 2 | 3868 | 16 |
| | Supercore 3 | 3869 | 17 |
| | Mecp2 | 3870 | 18, 192 |
| | CMVmini | 3871 | 19 |
| | CMVmini2 | 3872 | 20 |
| | miniCMVIE | 3873 | 21, 193 |
| | adML | 3874 | 22 |
| | hepB | 3875 | 23 |
| | RSV | 3876 | 54 |
| | hSyn | 3877 | 55 |
| | SV40 | 3878 | 56 |
| | hPGK | 3879 | 57 |
| | Jet | 3880 | 58, 72-74, 191 |
| | Jet + UsP intron | 3881 | 59, 75-77 |
| | hRLP30 | 3882 | 60 |
| | hRPS18 | 3883 | 61 |
| | CBA | 3884 | 62 |
| | CBH | 3885 | 63 |
| | CMV core | 3562 | 64, 198 |
| | U1a | 3886 | 177, 180, 181, 182, |
| | CpG-reduced U1a | 3887 | 178, 206 |
| | promoter | | |
| | CpG-depleted U1a | 3888 | 179, 205 |
| | promoter | | |
| | CpG-reduced UbC | 3889 | 184 |
| | promoter | | |
| | Strongly CpG-reduced | 3890 | 185 |
| | UbC promoter | | |
| | CpG-depleted UbC | 3891 | 186 |
| | promoter | | |
| | SFCp | 3892 | 194 |
| | miniSV40 | 3893 | 195 |
| | pJB42CAT5 | 3894 | 196 |
| | MLP | 3895 | 197 |
| | miniEF1α | 3896 | none |
| | hRPL13a | 3897 | none |
| | CK8e promoter | 3898 | 216 |
| | MHCK7 promoter | 3899 | 217 |
| | Desmin promoter | 3900 | 218 |
| | MHCK promoter | 3901 | 219 |
| | SP-301 | 3902 | none |
| | SpC5-12 | 3903 | none |
| 5′ NLS aa | 1X SV40 NLS | 3904 | |
| sequence | 4X SV40 NLS | 3905 | 121-123 |
| | 1X Cmyc NLS | 3906 | 83, 84, 89-102, 124-131, 135-137, 141-155, |
| | | | 157-174, 177-186, 188-198, 207-210 215- |
| | | | 220 |
| | 2X Cmyc NLS | 3907 | 127-129 |
| | 4X Cmyc NLS | 3908 | 130, 131 |
| | 6X Cmyc NLS | 3909 | 135-137, 142 |
| | 1X Nucleoplasmin NLS | 3910 | 132-134 |
| | 2X Nucleoplasmin NLS | 3911 | 138-140 |
| | 1X Cmyc 1X SV40 NLS | 3912 | none |
| | 1X Cmyc 2′ 1X SV40 | 3913 | none |
| | NLS | | |
| | 1X Cmyc 2′ NLS | 3914 | none |
| | 3X Cmyc 2′ NLS | 3915 | none |
| | 4X Cmyc 2′ NLS | 3916 | none |
| | 1X CPV NLS IN | 3917 | none |
| | 2X CPV NLS IN | 3918 | none |
| | 1X hBOVc NLS IN | 3919 | none |
| | 1X hBOVc NLS 2N | 3920 | none |
| | 1X SIRT NLS | 3921 | none |
| | 2X SIRT NLS | 3922 | none |
| | 1X Cmyc NLS 1X | 3923 | none |
| | BPSV40 NLS GGS | | |
| | 1X Cmyc NLS 1X | 3924 | none |
| | BPSV40 NLS PPPPG | | |
| | 1X Cmyc NLS 1X | 3925 | none |
| | BPSV40 NLS px330 PG | | |
| | 1X Cmyc NLS 1X | 3926 | none |
| | BPSV40 NLS (GGGS)2 | | |
| | PG | | |
| | 1X Cmyc NLS 1X | 3927 | none |
| | BPSV40 NLS P(GGGS)2 | | |
| | PG | | |
| | 1X Cmyc NLS 1X | 3928 | none |
| | BPSV40 NLS alpha PG | | |
| | 1X Cmyc NLS 1X | 3929 | none |
| | BPSV40 NLS PG | | |
| | 1X Cmyc GGS 1X SV40 | 3930 | none |
| | GGS | | |
| | 1X Cmyc PPP 1X SV40 | 3931 | none |
| | PG | | |
| | 1X Cmyc PG | 3932 | none |
| | 1X Cmyc (GGGS)3 | 3933 | none |
| | 1X Cmyc PPP | 3934 | none |
| | 1X Cmyc (GGGS)3 PPP | 3935 | none |
| | 1X SV40 PPP | 3936 | none |
| | 1X SV40 GGS | 3937 | none |
| CasX | CpG-free cMycNLS- | 3938 | 205, 206 |
| | Stx491-cMycNLS | | |
| 3′ NLS aa | 1X SV40 NLS | 3940 | 211-214 |
| sequence | 4X SV40 NLS | 3941 | 149 |
| | 6S SV40 NLS | 3942 | none |
| | 1X Cmyc NLS | 3939 | 141, 142, 150, 157-174, 177-186, 188-198, |
| | | | 207-210 |
| | 2X Cmyc NLS | 3943 | 151 |
| | 4x Cmyc NLS | 3944 | none |
| | 6x Cmyc NLS | 3945 | 152 |
| | 1X Nucleoplasmin NLS | 3946 | 119, 122, 125, 128, 130, 133, 136, 139, 153 |
| | 2X Nucleoplasmin NLS | 3947 | 120, 123, 126, 129, 131, 134, 137, 140, 154 |
| | 2x Nucleoplasmin 2x | 3948 | 155 |
| | SV40 NLS | | |
| | B19 NLS 1C | 3949 | none |
| | BoV NLS 3C | 3950 | none |
| | 1X SV40 GS 1X | 3951 | none |
| | Nuceloplasmin NLS | | |
| | GP vBPSV40 12 aa SV40 | 3952 | 143 |
| | NLS | | |
| | (GGGs)2vBPSV40 12 aa | 3953 | none |
| | SV40 | | |
| | 3′alphahelix vBPSV40 | 3954 | 144 |
| | 12aa SV40 | | |
| | GP SV40 GGS vBPSV40 | 3955 | none |
| | 12aa SV40 | | |
| | GP alpha helix Cmyc | 3956 | 145 |
| | NLS | | |
| | GP (GGGS)3 Cmyc NLS | 3957 | 146 |
| | GP SV40 PPP Cmyc | 3958 | 148 |
| | NLS | | |
| | GP Cmyc NLS | 3959 | 147 |
| | TGGGPGGGAAAGSGS- | 3960 | none |
| | 1xSV40-GS-Nuc | | |
| | TGGGPGGGAAAGSGS- | 3961 | none |
| | 1xSV40-GS | | |
| | PPPlinker 1xSV40 | 3962 | none |
| | PPPlinker | | |
| | GGSlinker 1xSV40 | 3963 | none |
| | PPPlinker | | |
| | PPPlinker 1xSV40 | 3964 | none |
| | GGSlinker 1xSV40 | 3965 | none |
| | GGSlinker 1xSV40 | 3966 | none |
| | (GGS)3linker | | |
| | GGSlinker 2xSV40 | 3967 | none |
| | (GGS)3linker 1xSV40 | 3968 | none |
| | GGS 1XSV40 | | |
| | PPP(GGGS)3linker | 3969 | none |
| | 1xCmyc | | |
| | PPPlinker 1xCmyc | 3970 | none |
| | PPP(GGGS)3linker | 3971 | none |
| | 1xCmyc | | |
| PTRE | WPRE1 | 3972 | 35, 38, 41, 72, 75, 78, 81, 83 |
| | WPRE2 | 3973 | 36, 39, 42, 73, 76, 79, 82, 84, 188-190 |
| | WPRE3 | 3974 | 34, 37, 40, 43, 74, 77, 80 |
| Poly(A) | bGH | 3975 | 1-23, 32, 33, 35-174, 177-181, 183-186, |
| signal | | | 188-198, 207-220 |
| | hGH | 3976 | 24 |
| | hGHshort | 3977 | 25 |
| | HSVTK | 3978 | 26 |
| | SynPolyA | 3979 | 27 |
| | SV40 | 3980 | 28 |
| | SV40short | 3981 | 29 |
| | bglob | 3982 | 30 |
| | bglobshort | 3983 | 31 |
| | SV40polyA late | 3984 | 34 |
| | CpG-depleted bGH- | 3985 | 182, 205, 206 |
| | polyA sequence | | |
| RNA | human U6 | 3986 | 1-31, 34-84, 103-157, 177-179, 182-186, |
| promoter | | | 188-198, 207, 209, 211-220 |
| | human U6 (rev comp) | 3987 | 208, 210 |
| | H1 | 3988 | 32, 158 |
| | 7SK | 3989 | 33 |
| | hU6 variant 1 | 3990 | 85, 89 |
| | hU6 variant 2 | 3991 | 86 |
| | hU6 variant 3 | 3992 | 87 |
| | hU6 variant 4 | 3993 | 88 |
| | hU6 variant 5 | 3994 | 90 |
| | hU6 variant 6 | 3995 | 91 |
| | hU6 variant 7 | 3996 | 92 |
| | hU6 variant 8 | 3997 | 93 |
| | hU6 variant 9 | 3998 | 94 |
| | hU6 variant 10 | 3999 | 95 |
| | hU6 variant 11 | 4000 | 96 |
| | hU6 variant 12 | 4001 | 97 |
| | hU6 variant 13 | 4002 | 98 |
| | hU6 variant 14 | 4003 | 99 |
| | hU6 variant 15 | 4004 | 100 |
| | hU6 variant 16 | 4005 | 101 |
| | hU6 variant 17 | 4006 | 102 |
| | H1 core | 4007 | 159 |
| | H1 core + 7SK hybrid 1 | 4008 | 160 |
| | H1 core + 7SK hybrid 2 | 4009 | 161 |
| | H1 core + 7SK hybrid 3 | 4010 | 162 |
| | H1 core + 7SK hybrid 4 | 4011 | 163 |
| | H1 core + 7SK hybrid 5 | 4012 | 164 |
| | H1 core + 7SK hybrid 6 | 4013 | 165 |
| | H1 core + 7SK hybrid 7 | 4014 | 166 |
| | H1 core + 7SK hybrid 8 | 4015 | 167 |
| | H1 core + 7SK hybrid 9 | 4016 | 168 |
| | H1 core + U6 hybrid 1 | 4017 | 169 |
| | H1 core + U6 hybrid 2 | 4018 | 170 |
| | H1 core + 7SK + U6 | 4019 | 171 |
| | hybrid 1 | | |
| | H1 core + U6 hybrid 3 | 4020 | 172 |
| | H1 core + 7SK + U6 | 4021 | 173 |
| | hybrid 2 | | |
| | H1 core + 7SK + U6 | 4022 | 174 |
| | hybrid 3 | | |
| | hU6 isoform 2 | 4023 | none |
| | hU6 isoform 3 | 4024 | none |
| | hU6 isoform 4 | 4025 | none |
| | hU6 isoform 5 | 4026 | none |
| | CpG-reduced U6 | 4027 | 180 |
| | promoter | | |
| | CpG-depleted U6 | 4028 | 181, 205, 206 |
| | promoter | | |
| | CpG-reduced hU6 | 4029 | none |
| | Isoform 2 | | |
| | CpG-depleted hU6 | 4030 | none |
| | Isoform 2 | | |
| | CpG-depleted hU6 | 4031 | none |
| | Isoform 3 | | |
| | CpG-depleted hU6 | 4032 | none |
| | Isoform 4 | | |
| | CpG-depleted hU6 | 4033 | none |
| | Isoform 5 | | |
| | mU6 | 4034 | none |
| | mU6 isoform | 4035 | none |
| | gorilla U6 | 4036 | none |
| | Saimiri U6 | 4037 | none |
| | Macaca U6 | 4038 | none |
| | Papio U6 | 4039 | none |
| | Rhinopithecus U6 | 4040 | none |
| | Mini Gorilla U6 | 4041 | none |
| | Mini Saimiri U6 | 4042 | none |
| | Mini Macaca U6 | 4043 | none |
| | Mini Papio U6 | 4044 | none |
| | Mini Rhinopithecus U6 | 4045 | none |
| 3′ ITR | AAV2 ITR | 4046 | 1-174, 177-186, 188-198, 207-220 |
| | CpG-depleted 3′ITR | 4047 | none |

### Example 28: AAV-Mediated Selective Expression of CasX in Rod and Cone Photoreceptors Results in Strong On-Target Activity at a Safe Harbor Locus in the Murine Retinae

Experiments were performed to demonstrate the ability of CasX to edit selectively rod and cone photoreceptors in the mouse retina by restricting its expression with a selective photoreceptor promoter, with a gRNA spacer targeting a safe harbor locus in the mouse genome. The correlation between editing and proteomic levels was demonstrated in a transgenic reporter mouse model that expressed GFP only in the rod photoreceptors.

### Materials and Methods

### Generation of AAV Plasmids and Viral Vectors:

CasX variant 491, flanked on either side by a c-MYC NLS, under the control of the various photoreceptor-specific promoters (listed in Table 46) based on the endogenous G-coupled Rhodopsin Kinase 1 (GRK1) promoter, and the gRNA guide variant 235 with spacer 35.2 (AGAAGAUGGGCGGGAGUCUU; SEQ ID NO: 9343) targeting the mouse ROSA26 locus under the U6 promoter, were cloned into a pAAV plasmid flanked with AAV2 ITR using standard cloning methods.

TABLE 46
Sequences of GRK1 promoter variants.

| Promoter | SEQ ID NO |
|---|---|
| GRK1(93) | 9344 |
| GRK1(94) | 9345 |
| GRK1(174) | 9346 |
| GRK1(199) | 9347 |
| GRK1(241) | 9348 |
| GRK1(292) | 9349 |
| GRK1(292)-SV40 | 9350 |
| GRK1-SV40 | 3718 |

AAV production and AAV titering were performed as described in Example 1.

Subretinal injections were performed in C57BL/6J mice as described in Example 16. Each mouse from the experimental groups was injected in one eye with 5E8 vg per eye. AAVs containing the GRK1-SV40 with a non-targeting (NT) gRNA served as an experimental control.

The processing of tissues, which were harvested three weeks post-injection, and subsequent NGS analysis were performed as described in Example 16. Briefly, gDNA was extracted using the Zymo Quick DNA/RNA™ miniprep Kit following the manufacturer's instructions and used for the amplification of the target amplicon at the ROSA26 locus. Target amplicons were sequenced and processed as described in Example 16.

ddPCR Analysis of AAV Genomes (Vg Dg):

The number of AAV viral genomes (vg) per diploid genome (dg) was determined in gDNA samples extracted from harvested tissues by ddPCR using the Bio-Rad QX200 Droplet Digital PCR instrument according to standard methods and following the manufacturer's protocol and guidelines. Briefly, ddPCR reactions containing the extracted gDNA samples were set up, serially diluted, and subjected to droplet formation using the droplet generator. Within each droplet, a PCR amplification reaction was performed using a primer-probe set specific to CasX, an indicator of the transgene, and mouse RPP30, an indicator of the mouse genome. Subsequently, droplet fluorescence was determined using a QX200 Droplet Reader with Bio-Rad QuantaSoft software. To calculate total vg/dg for each tissue, the total quantified copy amount for CasX was divided by the copy amount calculated for RPP30, and then divided by 2 (diploid genome per cell).

### Results

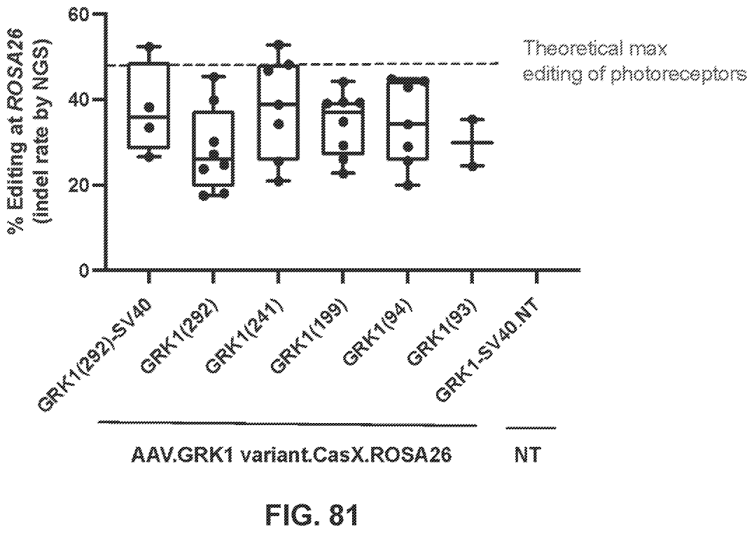

Editing levels at the ROSA26 locus were quantified in retinae harvested from mice injected sub-retinally with AAVs expressing CasX 491 under the control of various engineered retinal promoters (listed in Table 46) to identify promoters driving the strongest levels of CasX-mediated editing in the photoreceptors. FIG. 81 is a box plot that shows the quantification of these editing levels at the ROSA26 locus for the indicated GRK1 promoter variants. The data demonstrate that use of the GRK1 promoter variants to drive CasX expression resulted in similar levels of editing (˜30-38%). Of the promoter variants tested, AAVs containing the GRK1(292)-SV40 and GRK1(241) promoter variants yielded the highest average editing levels, achieving 37.73±10.89% and 38.27±11.98% editing respectively (FIG. 81). As illustrated in FIG. 81, use of GRK1(292)-SV40 and GRK1(241) promoters resulted in the maximum editing that could be achieved in the photoreceptors (dashed line, which indicates the theoretical maximum editing of photoreceptors that can be achieved with optimal transduction).

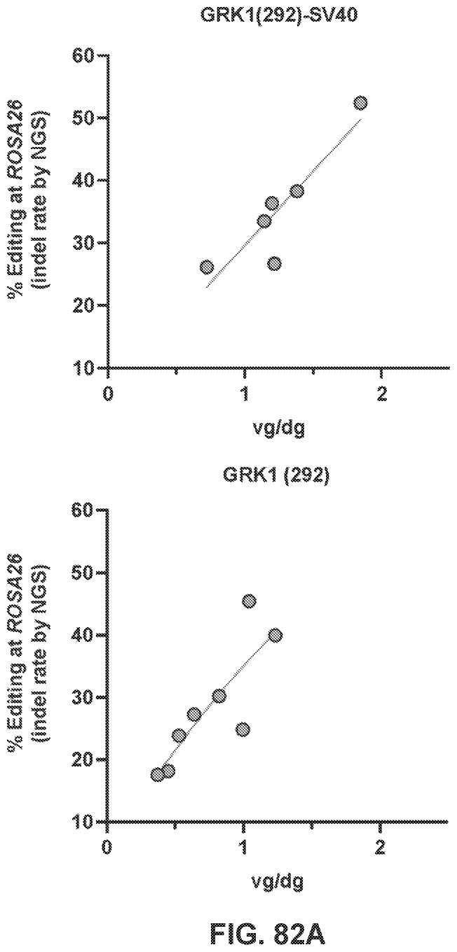

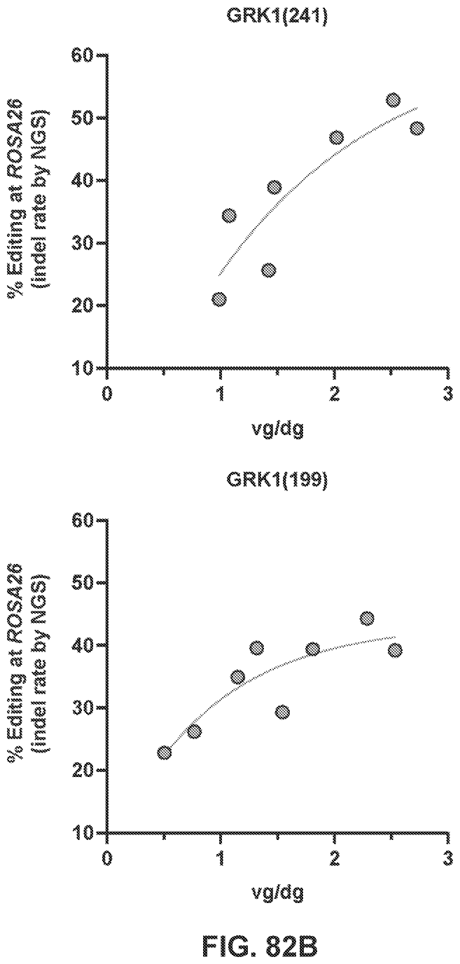

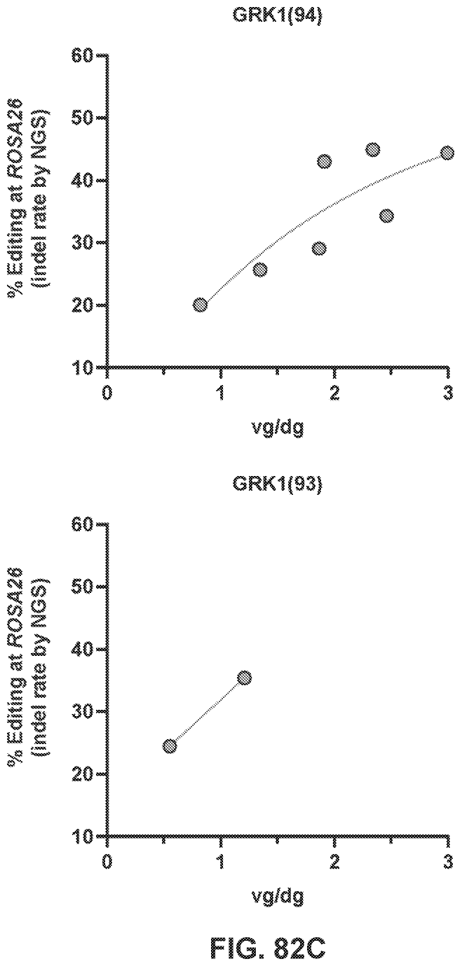

Additional analyses were performed by correlating editing levels achieved when using a particular promoter variant with the vg/dg quantification, to account for potential variation in AAV delivery. The editing profile for each promoter variant was plotted with the corresponding vg/dg value, and a nonlinear regression curve was fitted to assess the correlation (FIGS. 82A-82C). The data demonstrate that there is an overall positive correlation between AAV dose (vg/dg) and percent editing, such that higher amount of AAV delivered would correlate with higher editing. Slope values were calculated for each regression plot, and the calculations are displayed in Table 47. Analysis of the slope values revealed that an increase from 1 vg/dg to 2 vg/dg for AAVs containing the GRK1(292)-SV40 or GRK1(292) promoter resulted in the highest incremental change in editing levels compared to the incremental changes achieved for the shorter promoter variants (Table 47, FIGS. 82A-82C). Furthermore, higher variability in editing levels was observed with use of the shorter promoter variants, especially with use of GRK1(199) and GRK1(94), indicated by the higher standard deviation values calculated for the corresponding slopes of the curves (Table 47, FIGS. 82A-82C). The data also show that saturation in editing was achieved when >1.5vg/dg, given the flattening of the curve observed (FIGS. 82A-82C). Interestingly, use of the GRK1(93) promoter appeared to exhibit stronger editing kinetics compared to the GRK1(199) and GRK1(94) promoters, given the higher slope value observed (Table 47).

TABLE 47
Slope values calculated from the nonlinear
regression curves in FIGS. 82A-82C.

| Promoter | Slope value | Standard deviation of slope |
|---|---|---|
| GRK1(292)-SV40 | 23.78 | 5.809 |
| GRK1(292) | 26.65 | 8.124 |
| GRK1(241) | 15.17 | 11.78 |
| GRK1(199) | 8.792 | 21.35 |
| GRK1(94) | 11.44 | 12.02 |
| GRK1(93) | 16.6 | 15.31 |

The results from these experiments demonstrate proof-of-concept that CasX, driven by the various photoreceptor-specific promoters with the targeting gRNA, can achieve editing in the photoreceptor cells of the retinae when delivered by AAVs via subretinal administration. Variable levels of editing were achieved when using the different promoter variants. Furthermore, given the limited cargo capacity of the AAV transgene, use of a shorter tissue-specific promoter to drive sufficient CasX expression to induce editing would be especially beneficial in the context of a dual-guide AAV vector.

### Example 29: Demonstration that Varying the Placement and Orientation of the gRNA Promoter in the CasX:Dual-gRNA System Expressed from an all-In-One AAV Vector can Affect Editing of the Target Locus

The experiments in Example 9 showed that the CasX:dual-gRNA system packaged and delivered within a single AAV was able to edit the target gene. Here, experiments were performed to demonstrate that placement and orientation of the gRNA promoters within the AAV transgene to drive expression of dual gRNAs can affect the efficiency of the dual-cut editing of a target locus. Within the AAV plasmid, gRNA promoters could be placed upstream, downstream, or flanking the CasX construct and could be in a forward or reverse orientation. The various configurations of the dual-gRNA transcriptional units relative to the CasX construct within the AAV transgene are illustrated in FIGS. 38-39 and FIG. 75.

### Materials and Methods

AAV plasmid constructs were generated and cloned into a pAAV plasmid flanked with AAV2 ITRs using standard molecular cloning methods as described in Example 1. Briefly, dual-gRNA AAV plasmids were generated to express CasX variant 491 driven by the ubiquitous UbC promoter and two gRNA transcriptional units that each expressed a Pol III U6 promoter-guide scaffold 235-a specific spacer combination (spacer 12.7 targeting the tdTomato locus (SEQ ID NO: 4049) and/or a non-targeting spacer. In this example, the two gRNA transcriptional units were cloned relative to the CasX construct using configuration #1, #2, and #4 (illustrated in FIGS. 38-39) and tested. Table 48 below shows the combinations of spacers tested for each of the three configurations of dual gRNA units relative to the CasX construct.

TABLE 48
Combinations of a tdTomato-targeting spacer (12.7) and a non-targeting
(NT) spacer tested in this example in configuration #1, #2, and
#4 (illustrated in FIGS. 38-39) of dual gRNA units relative to
the CasX 491 construct. The “R” preceding the spacer denotes
the reverse orientation of the transcription of the indicated gRNA unit.

| Configuration # | Specific spacer combination tested |
|---|---|
| #1 | NT-CasX 491-NT |
| #1 | 12.7-CasX 491-NT |
| #1 | NT-CasX 491-12.7 |
| #1 | 12.7-CasX 491-12.7 |
| #4 | R.NT-CasX491-NT |
| #4 | R.12.7-CasX 491-NT |
| #4 | R.NT- CasX 491-12.7 |
| #4 | R.12.7- CasX 491-12.7 |
| #2 | CasX 491-NT-NT |
| #2 | CasX 491-12.7-NT |
| #2 | CasX 491-NT-12.7 |
| #2 | CasX-12.7-12.7 |

AAV nucleofection of tdTomato mNPCs was performed as described in Example 1. Briefly, 125 ng of AAV plasmid encoding for XAAVs expressing the CasX:dual-gRNA system with the various configurations listed in Table 48 were nucleofected mNPCs. Five days post-nucleofection, mNPCs were harvested for editing analysis at the tdTomato locus by FACS, as described in Example 1. For comparison, AAV plasmid encoding for XAAVs expressing CasX 491 with a single gRNA transcriptional unit using spacer 12.7 was also used in this example.

AAV production and AAV titering were performed as described in Example 1.

AAV Transduction of tdTomato mNPCs, Followed by Flow Cytometry:

˜10,000 mNPCs were seeded per well in PLF-coated 96-well plates; 48 hours later, seeded cells were transduced with AAVs expressing the CasX:dual-gRNA system of various configurations (Table 48). All viral infection conditions were performed in triplicate, with a normalized number of viral genomes (vg) among experimental vectors, in a series of three-fold dilution of MOI ranging from −1E5 to 1E3 vg/cell. Five days post-transduction, XAAV-treated mNPCs were harvested for editing analysis at the tdTomato locus by FACS, as described earlier in Example 1. For comparison, AAVs expressing CasX 491 with a single gRNA transcriptional unit using spacer 12.7 were also assayed in this example.

### Results

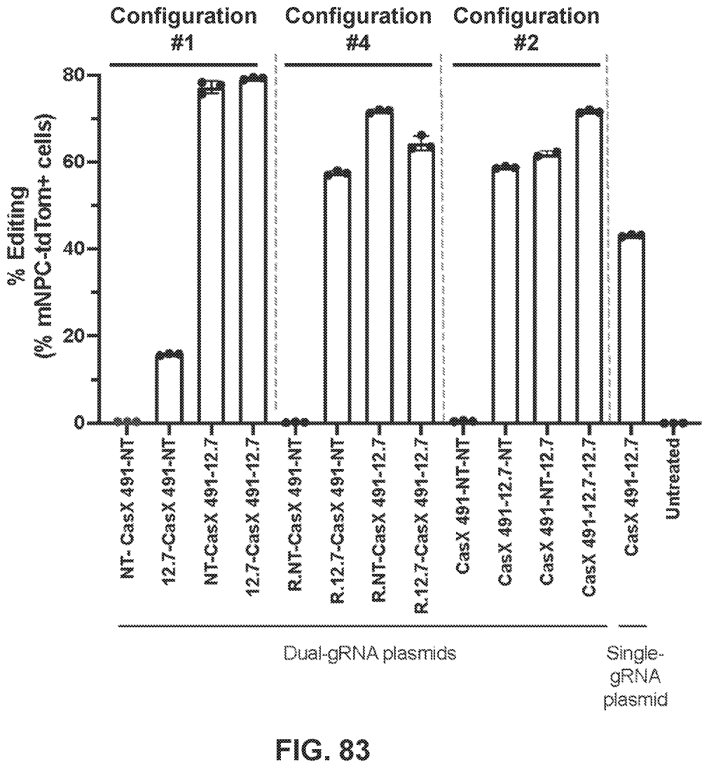

tdTomato mNPCs were nucleofected with AAV plasmids encoding for dual-guide AAVs expressing the CasX:dual-gRNA system in various vector configurations with different spacer combinations of spacer 12.7 or a non-targeting spacer (listed in Table 48). Editing levels at the tdTomato locus were subsequently assessed to determine the difference in editing level achieved and driven by a spacer in a particular orientation and position, and the results are illustrated in FIG. 83. The data indicate that for configuration #1 (FIG. 38), the editing levels achieved appeared to be primarily driven by the second gRNA positioned on the 3′ end of the CasX construct in the sense orientation, since use of the NT-CasX 491-12.7 AAV construct resulted in ˜80% editing, while use of the 12.7-CasX 491-NT construct resulted in ˜20% editing (FIG. 83). In addition, use of the NT-CasX 491-12.7 construct resulted in similar levels of editing as use of the AAV construct with two 12.7 spacers (12.7-CasX 491-12.7; FIG. 83). The data further showed that positioning and orienting the gRNA units in configurations #4 and #2 (FIG. 38) appeared to induce similar levels of editing overall between the two gRNA units within an AAV transgene (FIG. 83).

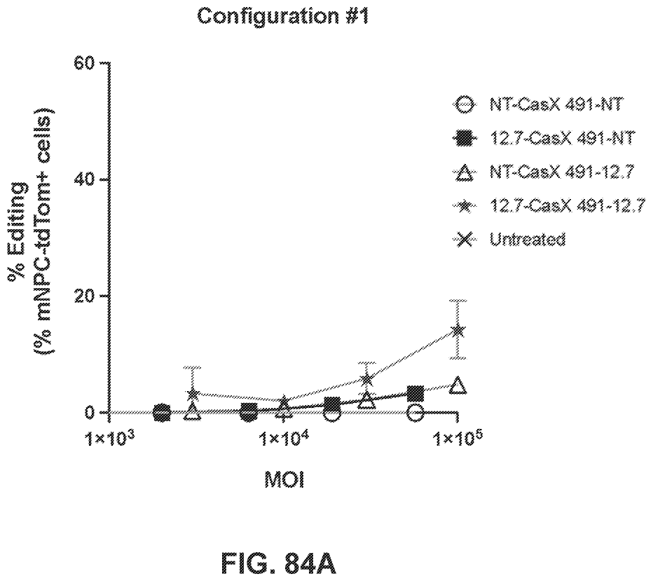

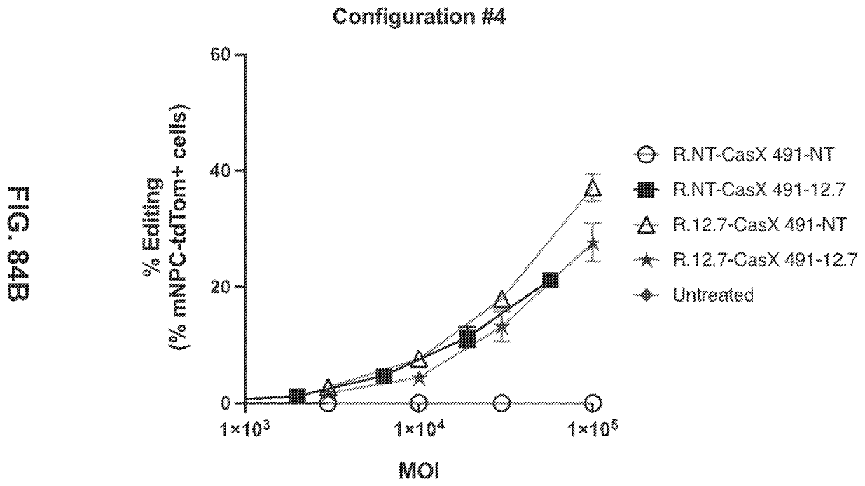

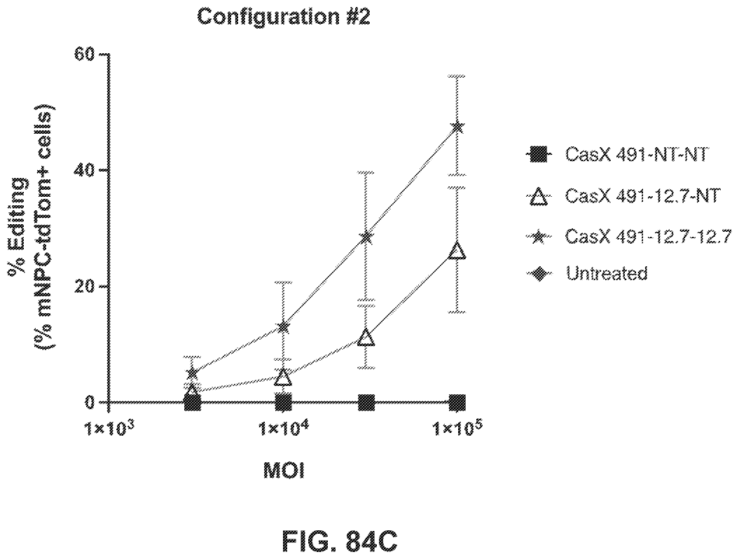

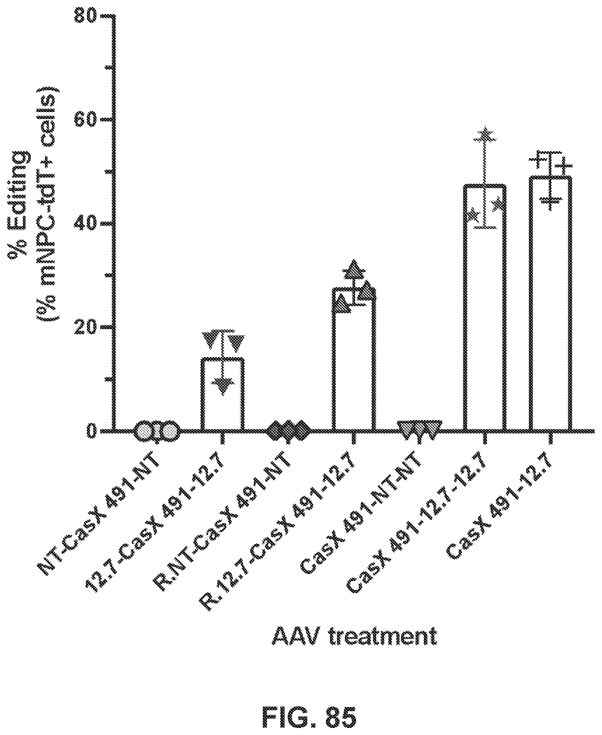

tdTomato mNPCs were also transduced with dual-guide AAVs expressing the CasX:dual-gRNA system in configurations #1, #2, and #4 (FIGS. 38-39) with different spacer combinations (Table 48) at varying MOIs, and editing levels were subsequently assessed, with the resulted portrayed in FIGS. 84A-84C. The data demonstrate that for configuration #1, each gRNA unit was able to achieve similar levels of editing, when comparing the editing levels induced by AAVs containing the 12.7-CasX 491-NT construct with those achieved by AAVs containing the NT-CasX 491-12.7 construct (FIG. 84A). Notably, AAVs containing the 12.7-CasX 491-12.7 construct induced comparatively higher editing than either of the spacer 12.7-NT combination (FIG. 84A). For configuration #4, AAVs containing the R.12.7-CasX 491-NT construct appeared to achieve a slightly higher editing level at the highest MOI tested (˜1E5 vg/cell), suggesting that the gRNA placed 5′ of the CasX construct and in antisense orientation was more active than its counterpart positioned 3′ to the CasX construct in the sense orientation (FIG. 84B). Interestingly, AAVs containing the R12.7-CasX 491-12.7 construct did not induce the highest level of editing out of all the spacer combinations tested for configuration #4, suggesting a saturation in editing levels achieved given the limitation in gRNA expression (FIG. 84B). Lastly, for configuration #2, the data demonstrate that at the highest MOI tested of 1E5; XAAVs with the CasX 491-12.7-NT construct induced ˜20% editing and XAAVs with the CasX 491-12.7-12.7 induced ˜40% editing, suggesting that each gRNA in either position in the sense orientation was able to drive a similar level of editing as its partner (FIG. 84C). A comparison of AAVs expressing a CasX:dual-guide system with two 12.7 spacers in configuration #1, #2, and #4 revealed that use of AAVs expressing the CasX:dual-gRNA system in configuration #2 induced the highest level of editing (˜47.6±8%), compared to −27.7±3.3% editing and ˜14.3±5% editing for configuration #4 and #1 respectively (FIG. 85).

The results from these experiments demonstrate that use of gRNAs in different positions and orientations relative to the CasX encoding construct within the AAV transgene can achieve efficient dual-cut editing at the target locus. Furthermore, varying the specific position and orientation of the gRNA unit can affect the editing efficiency.

### Example 30: Further Demonstration that Altering the Placement and Orientation of the gRNA Promoter in the CasX:Dual-gRNA System Expressed from an all-In-One AAV Vector Affects Editing of the Target Locus

Experiments were performed to demonstrate further that placement and orientation of the gRNA promoters within the AAV transgene to drive expression of dual gRNAs can affect the efficiency of the dual-cut editing of a target locus. As discussed in Examples 9 and 31, gRNA promoters may be placed upstream, downstream, or flanking the CasX construct and may be in a forward or reverse orientation. The various configurations of the dual-gRNA transcriptional units relative to the CasX construct within the AAV transgene are illustrated in FIGS. 38-39 and FIG. 75.

### Materials and Methods

AAV plasmid constructs were generated and cloned into a pAAV plasmid flanked with AAV2 ITRs using standard molecular cloning methods as described in Example 1. Briefly, dual-gRNA AAV plasmids were generated to express CasX variant 515 driven by the U1A promoter and two gRNA transcriptional units that each expressed a Pol III U6 promoter-guide scaffold 235-a specific spacer combination (spacer 31.63 targeting the AAVS1 locus and spacer 7.37 targeting the B2M locus). In this example, efforts were made to clone the two gRNA transcriptional units relative to the CasX construct using all 12 configurations (illustrated in FIGS. 38-39, and FIG. 75). However, efforts to clone the constructs using configurations #3, #6, #9 and #11 were not successful. Furthermore, two additional configurations, #5 and #12, were not cloned. Therefore, dual-guide AAV constructs having the configurations #1-2, #4, #7-8, and #10 were further evaluated in a cell-based assay. Table 49 below shows the combinations of spacers for each of the 12 configurations of dual gRNA units relative to the CasX construct. Table 50 below shows the sequences of AAV elements with varying positions and orientations of the gRNA promoter to drive gRNA expression in a dual-guide context. Table 51 provides the sequences of AAV elements for single-guide AAV constructs that were used as controls.

TABLE 49
Combinations of a B2M-targeting spacer (7.37) and an AAVS1-targeting
spacer (31.63) spacer designed using all 12 configurations (FIGS.
38-39, and FIG. 75) of dual gRNA units relative to the CasX 515
construct. The “R” preceding the spacer denotes the reverse
orientation of the transcription of the indicated gRNA unit.

| Configuration | Specific spacer | AAV |
|---|---|---|
| # | combination tested | construct ID |
| #1 | 31.63-CasX 515-7.37 | 299 |
| #2 | CasX 515-31.63-7.37 | 300 |
| #3 | CasX 515-31.63-R.7.37 | N/A |
| #4 | R.31.63-CasX 515-7.37 | 301 |
| #5 | 31.63-CasX515-R.7.37 | N/A |
| #6 | 7.37-R.31.63-CasX 515 | N/A |
| #7 | 7.37-31.63-CasX 515 | 302 |
| #8 | R.7.37-R.31.63-CasX 515 | 303 |
| #9 | CasX 515-R.31.63-7.37 | N/A |
| #10 | CasX 515-R.31.63-R.7.37 | 304 |
| #11 | R.7.37-31.63-CasX 515 | N/A |
| #12 | R.31.63-CasX 515-R.7.37 | N/A |

TABLE 50
Sequences of AAV elements for constructs with varying positions
and orientations of gRNA transcriptional units. Note that
the table does not contain buffer sequences. “Rev comp”
denotes the reverse complementary sequence.

| | | | Length of |
|---|---|---|---|
| AAV | | SEQ | Component |
| construct ID | Component Name | ID NO | (bp) |
| 299 through 304 | 5′ ITR | 17 | 130 |
| | UlA promoter | 3722 | 252 |
| | cMyc NLS | 9290 | 27 |
| | CasX 515 | 9352 | 2934 |
| | cMyc NLS | 9353 | 27 |
| | bGH poly(A) | 3401 | 208 |
| 299 through 302 | U6 promoter (Fwd) | 3563 | 241 |
| 299 through 302 | Scaffold 235 (Fwd) | 3638 | 99 |
| 299, 300 | AAVS1 Spacer 31.63 (Fwd) | 9354 | 20 |
| 299 through 302 | B2M Spacer 7.37 (Fwd) | 9360 | 20 |
| 301 through 304 | U6 promoter (Rev Comp) | 3987 | 241 |
| 301 through 304 | Scaffold 235 (Rev Comp) | 9293 | 99 |
| 301 through 304 | AAVSI Spacer 31.63 (Rev | 9367 | 20 |
| | Comp) | | |
| 303, 304 | B2M Spacer 7.37 (Rev | 9368 | 20 |
| | Comp) | | |
| 299 through 304 | 3′ ITR | 3701 | 141 |

TABLE 51
Sequences of AAV elements for constructs with single gRNA transcriptional
units.

| | | DNA Sequence | Length of |
|---|---|---|---|
| AAV construct ID | Component Name | or SEQ ID NO | Component (bp) |
| 305 through 307 | 5′ ITR | 17 | 130 |
| | buffer sequence | 3684 | 23 |
| | UlA promoter | 3722 | 252 |
| | buffer sequence | 9302 | 18 |
| | Kozak | GCCACC | 6 |
| | CasX 515 | 9352 | 2934 |
| | stop codon | TGA | 3 |
| | buffer sequence | 9366 | 30 |
| | bGH poly(A) | 3401 | 208 |
| | buffer sequence | GGTACCGT | 8 |
| | U6 promoter | 3563 | 241 |
| | buffer sequence | GAAACACC | 8 |
| | Scaffold 235 | 3638 | 99 |
| 305 | AAVS1 spacer (31.63) | 9354 | 20 |
| 306 | B2M spacer (7.37) | 9360 | 20 |
| 307 | Non-targeting spacer | 9318 | 20 |
| | (0.0) | | |
| 305 through 307 | buffer sequence | 3700 | 17 |
| | 3′ ITR | 3701 | 141 |

### AAV Production and Titering:

The resulting AAV constructs were used to produce AAVs following similar methods as described in Example 18. AAV titering was performed by ddPCR following similar methods as described in Example 31. Two separate primer-probe sets were used for AAV titering by ddPCR: a primer-probe set specific to BGH and another primer-probe set specific to CasX.

### AAV Transduction of iNs (Induced Neurons) In Vitro:

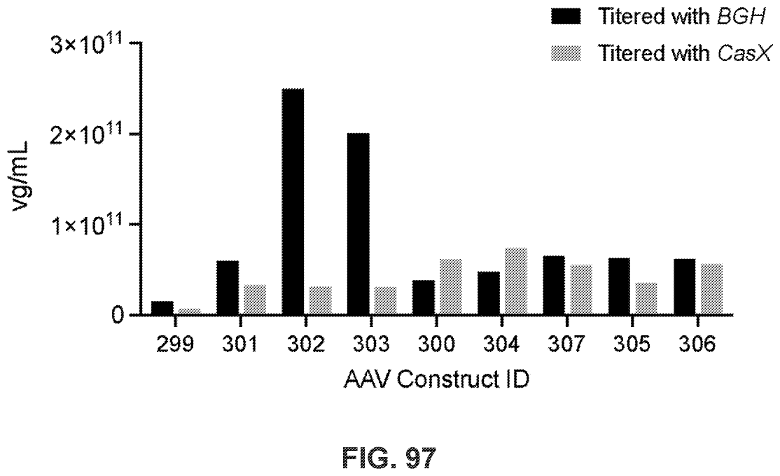

Produced AAVs were used to transduce human iNs to assess editing level at the AAVS1 and B2M loci. AAV transduction of iNs was performed following similar methods as described in Example 32. In one replicate, cells were transduced at the following MOIs: 1.3E4, 4.33E3, or 1.44E3 vg/cell. In the second replicate, cells were transduced at the following MOIs: 1E4, 3E3, or 1E3 vg/cell. The following three constructs were included as experimental controls: 1) a single-guide AAV construct with the AAVS1-targeting spacer (AAV construct ID #305); 2) a single-guide AAV construct with the B2M-targeting spacer (AAV construct ID #306); 3) an AAV construct with a non-targeting spacer (AAV construct ID #307). The results of this experiment are illustrated in FIGS. 97-101.

### Results

In this example, dual-guide AAV constructs were designed to harbor each of the 12 configurations shown in FIGS. 38-39 and FIG. 75. As discussed above, efforts to clone the dual-guide constructs using configurations #3, #6, #9 and #11 were not successful. Furthermore, constructs using two additional configurations, #5 and #12, were not cloned. Therefore, AAVs were produced using the remaining dual-guide AAV constructs having the configurations #1-2, #4, #7-8, and #10 and titered via ddPCR using two independent primer-probe sets: one to BGH and one to CasX. Titering results are shown in FIG. 97. The data show that titering using the BGH probe was unusually high for AAV particles produced using AAV construct ID #302 and #303 compared to titering levels using the CasX probe. One potential explanation for this observed titering discrepancy is that AAV particles produced using AAV construct ID #302 and #303 primarily packaged truncated AAV transgenes; however, additional experiments are needed to assess this phenomenon further. As a result, MOIs for AAV transduction experiments were calculated based on titering results determined using the CasX primer-probe set.

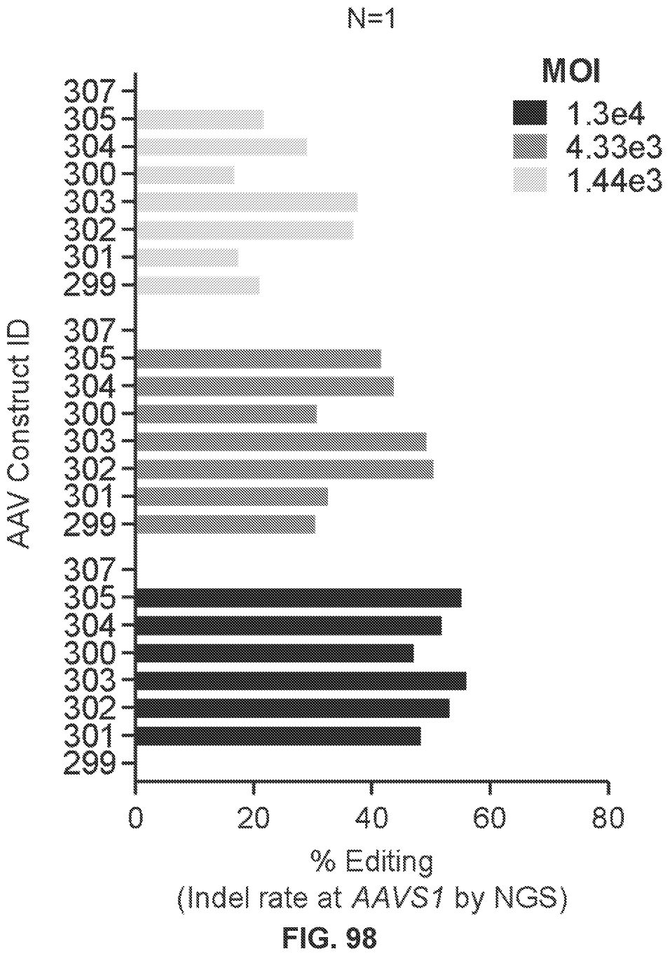

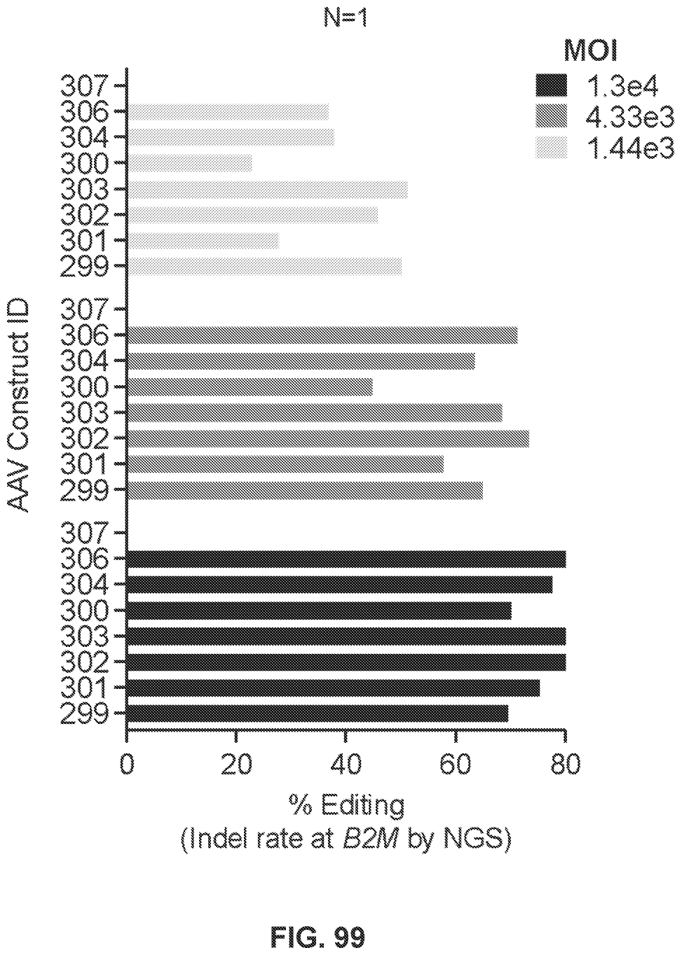

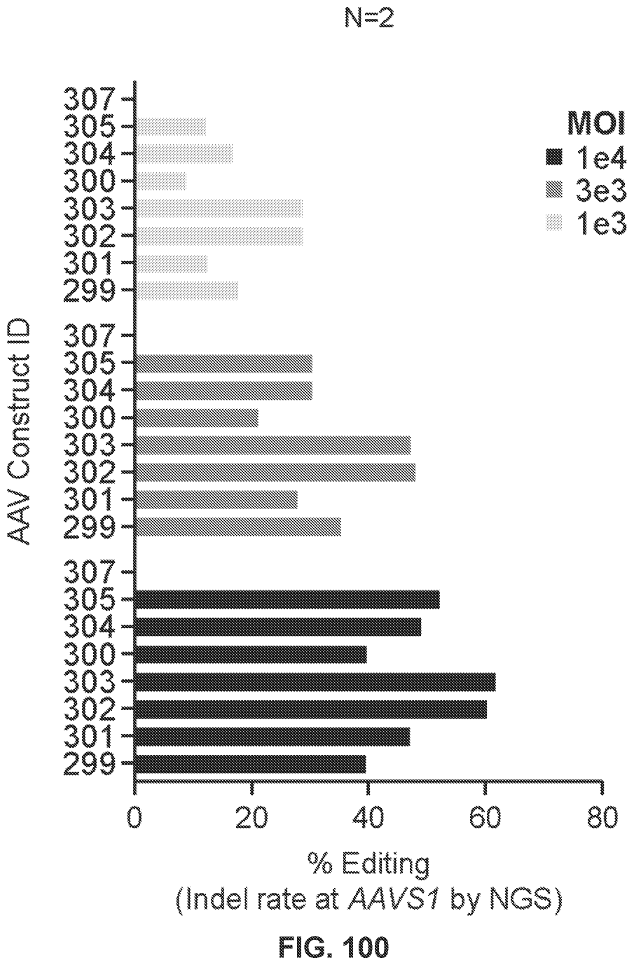

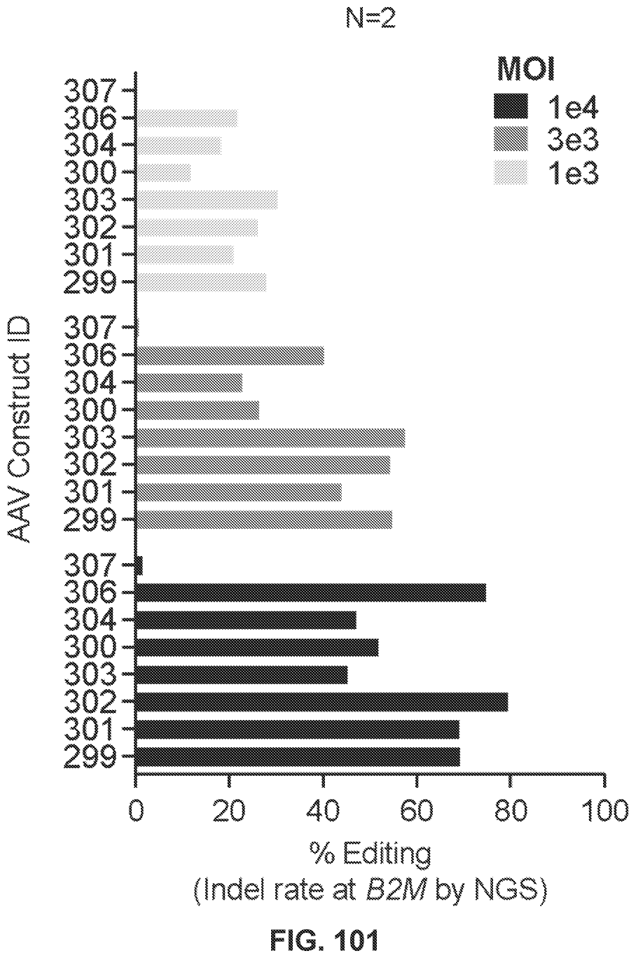

Human iNs were transduced with AAVs containing the transgene encoding for dual-guide AAVs expressing the CasX:dual-gRNA system in various vector configurations with different spacer combinations of spacer 31.63 and spacer 7.37 (listed in Table 49). Editing levels at the AAVS1 and B2M loci were subsequently assessed to determine the difference in editing level achieved and driven by a spacer in a particular orientation and placement, and the results are illustrated in FIGS. 98-99 for the first replicate and FIGS. 100-101 for the second replicate. The data show that all tested constructs having the 6 configurations were able to edit the two target loci successfully, albeit at varying editing rates. Further experiments are performed to assess the effects of each dual-guide orientation on CasX expression and packaging of AAV transgenes (full-length vs. truncations).

The results from these experiments demonstrate that use of gRNAs in different positions and orientations relative to the CasX encoding construct within the AAV transgene can achieve efficient dual-cut editing at the target locus. Furthermore, varying the specific position and orientation of the gRNA unit can affect AAV production and editing efficiency.

### Example 31: Assessment of U6 Isoforms as Alternative Guide RNA Promoters

Experiments were performed to assess various U6 isoforms as alternative gRNA promoters. Utilization of an alternative U6 gRNA promoter would enable the following: 1) in the context of a dual-guide AAV construct, mitigate the potential for recombination risk during cloning while also maintain a balance in editing levels between the two gRNA transcriptional units; 2) increase overall AAV editing efficiency and potency; and 3) result in the identification of smaller U6 promoter alternatives given the limited capacity of the AAV transgene.

### Materials and Methods

AAV plasmid constructs were generated and cloned into a pAAV plasmid flanked with AAV2 ITRs using standard molecular cloning methods as described in Example 1. Briefly, AAV plasmids were generated to express CasX protein 515 driven by the U1A promoter with a bGH poly(A) signal sequence and a gRNA transcriptional unit that expressed a Pol III promoter-guide scaffold 235 with spacer 31.63 targeting the AAVS1 locus. In this example, various U6 isoforms were assessed as Pol III promoter variants, which were cloned downstream relative to the CasX construct in the AAV plasmid; the sequences of the U6 isoforms tested are shown in Table 52. Table 53 shows the sequences of the constructs encoding for full-length AAV transgene used in this example.

TABLE 52
Sequences of Pol III promoters assessed in this example.

| SEQ | Pol | AAV | Length of |
|---|---|---|---|
| ID NO: | III promoter | construct ID | Promoter (bp) |
| 3599 | hU6 isoform 2 | 233 | 249 |
| 4743 | CpG-depleted hU6 | 235 | 249 |
| | Isoform 2 | | |
| 4029 | CpG-reduced hU6 | 234 | 249 |
| | Isoform 2 | | |
| 3600 | hU6 isoform 3 | 236 | 249 |
| 3744 | CpG-depleted hU6 | 237 | 249 |
| | Isoform 3 | | |
| 4025 | hU6 isoform 4 | 238 | 249 |
| 4032 | CpG-depleted hU6 | 239 | 249 |
| | Isoform 4 | | |
| 3602 | hU6 isoform 5 | 240 | 249 |
| 3746 | CpG-depleted hU6 | 241 | 244 |
| | Isoform 5 | | |
| 3563 | hU6 isoform 1 | 242 | 241 |
| 3603 | mU6 | 243 | 304 |
| 9351 | mU6 with G | 244 | 305 |

TABLE 53
Sequences of AAV constructs encoding for the transgene used in this example.

| | | DNA Sequence | Length of |
|---|---|---|---|
| AAV construct ID | Component Name | or SEQ ID NO | Component (bp) |
| 233 through 244 | 5′ ITR | 17 | 130 |
| | buffer sequence | 3684 | 23 |
| | UlA promoter | 3722 | 252 |
| | buffer sequence | 9302 | 18 |
| | Kozak | GCCACC | 6 |
| | start codon | ATGGCC | 6 |
| | c-MYC NLS | 9290 | 27 |
| | linker | TCTAGA | 6 |
| | CasX 515 | 9352 | 2934 |
| | linker | GGATCC | 6 |
| | c-MYC NLS | 9353 | 27 |
| | stop codon | TAA | 3 |
| | buffer sequence | 3695 | 30 |
| | bGH poly(A) | 3401 | 208 |
| | buffer sequence | GGTACCGT | 8 |
| | U6 promoter variants | See sequences | — |
| | | listed in | |
| | | Table 52 above | |
| | Scaffold 235 | 3638 | 99 |
| | AAVS1 spacer (31.63) | 9354 | 20 |
| | buffer sequence | 3700 | 17 |
| | 3′ ITR | 3701 | 141 |

*Components are listed in a 5′ to 3′ order within the constructs

AAV production was performed following similar methods as described in Example 1. AAV titering was performed by ddPCR according to standard methods and following the manufacturer's protocol and guidelines. Briefly, ddPCR reactions containing the AAV viral samples were set up, serially diluted, and subjected to droplet formation using the droplet generator. Within each droplet, a PCR amplification reaction was performed using a primer-probe set specific to bGH, an indicator of the AAV transgene. Subsequently, droplet fluorescence was determined using a QX200 Droplet Reader with Bio-Rad QuantaSoft software.

### AAV Transduction of iNs (Induced Neurons) In Vitro:

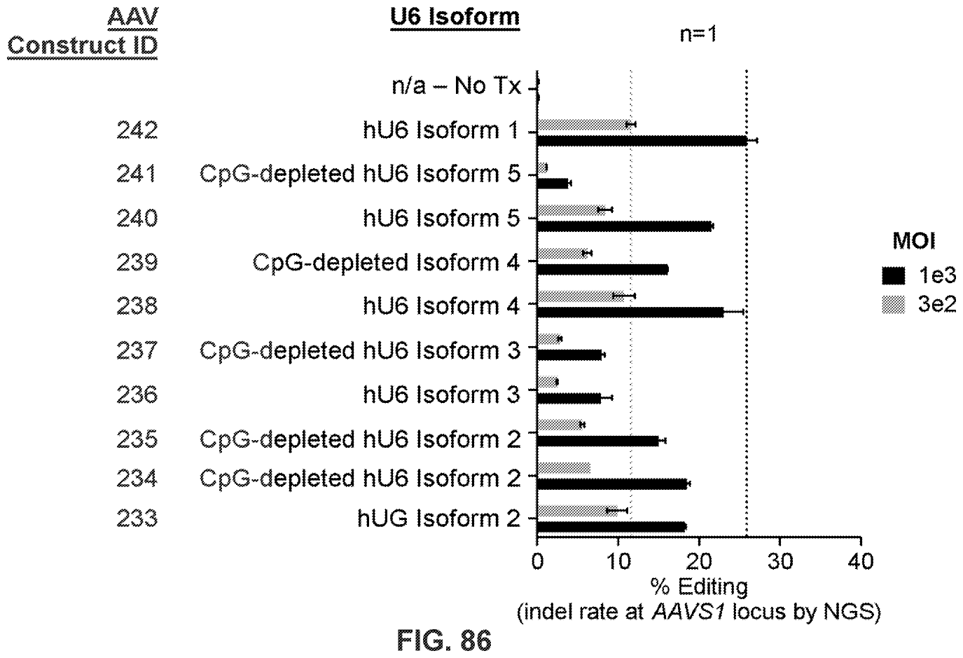

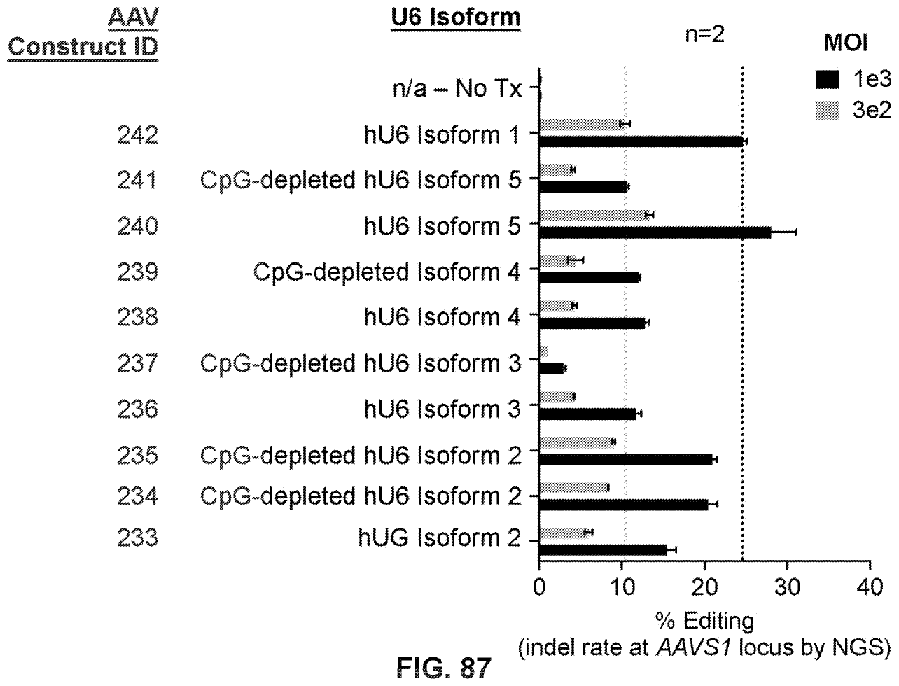

In a first transduction experiment, ˜50,000 iNs per well were seeded on Matrigel-coated 96-well plates 7 days prior to transduction. AAVs expressing the CasX:gRNA system, containing various U6 promoters listed in Table 52, were then diluted in neuronal plating media and added to cells. Cells were transduced at two MOIs (1E3 or 3E2 vg/cell). 7 days post-transduction, cells were lifted using lysis buffer, and gDNA was harvested and prepared for editing analysis at the AAVS1 locus using NGS following similar methods as described in Example 18. Samples that were not transduced with AAV were included as controls. Wild-type human U6 (hU6 isoform 1) served as the benchmark for comparison. Two replicates were performed, and the results of this experiment are shown in FIGS. 86 and 87.

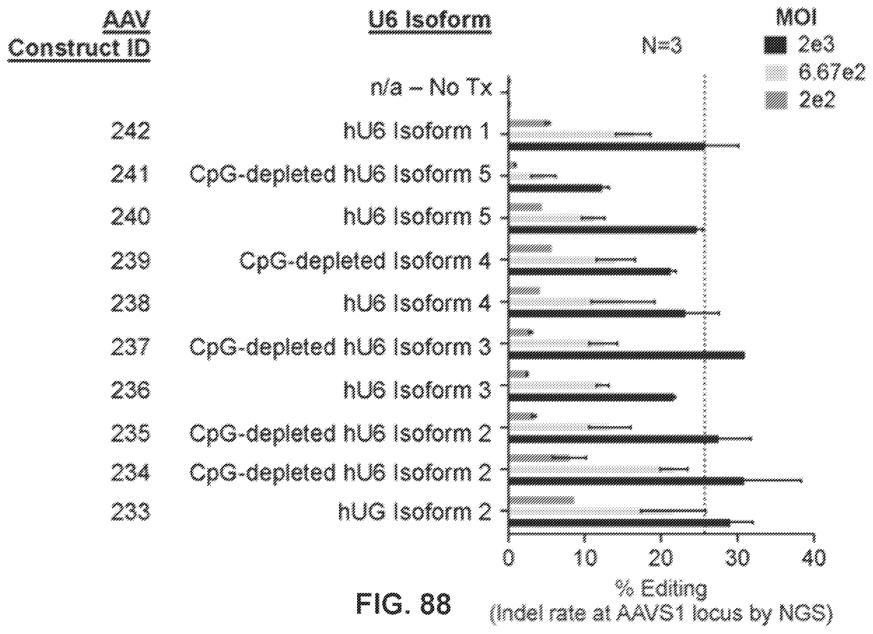

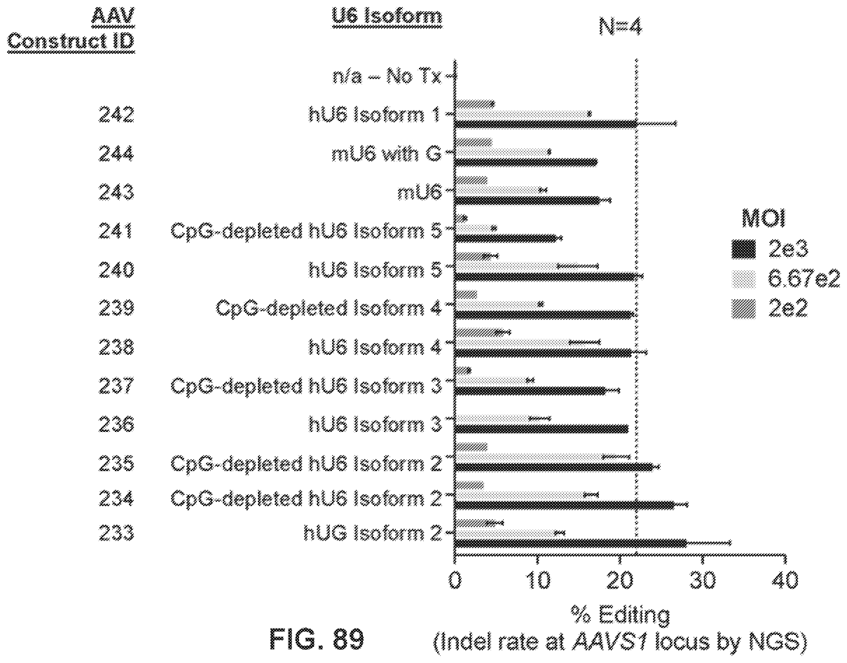

In a second AAV transduction experiment, ˜50,000 iNs per well were seeded on Matrigel-coated 96-well plates 14 days prior to transduction. Cells were transduced at three MOIs (2E3, 6.67E2, or 2.2E2 vg/cell). Cells were harvested for editing analysis at the AAVS1 locus 7 days post-transduction using NGS following similar methods as described in Example 18. Samples that were not transduced with AAV were included as controls. Wild-type human U6 (hU6 isoform 1) served as the benchmark for comparison. Two replicates were performed, and the results of this experiment are shown in FIGS. 88 and 89.

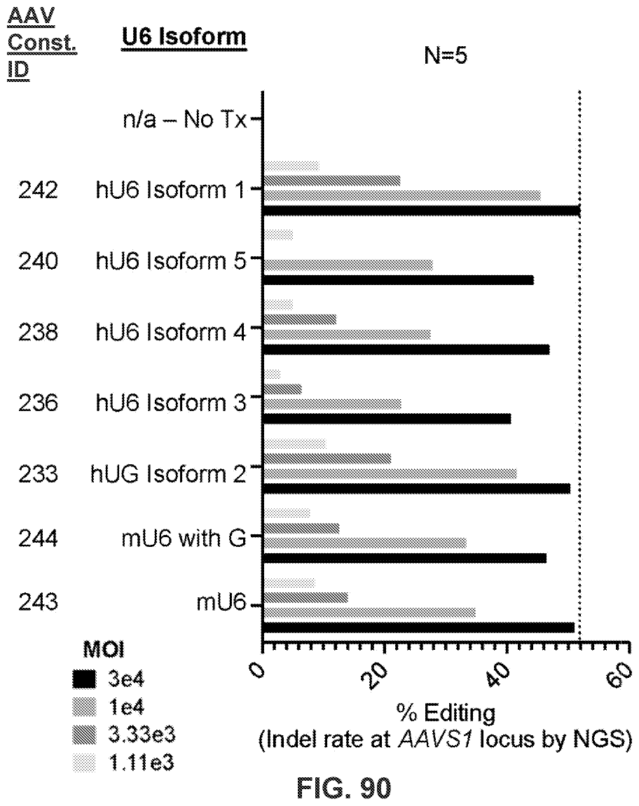

In a third AAV transduction experiment, ˜50,000 iNs per well were seeded on Matrigel-coated 96-well plates 14 days prior to transduction. Cells were transduced at the following MOIs: 3E4, 1E3, 3.33E3, and 1.11E3. Cells were harvested for editing analysis at the AAVS1 locus 7 days post-transduction using NGS following similar methods as described in Example 18. Samples that were not transduced with AAV were included as controls. Wild-type human U6 (hU6 isoform 1) served as the benchmark for comparison. One replicate was performed, and the results of this experiment are shown in FIG. 90.

### Results

Three sets of experiments were performed in iNs to assess various U6 isoforms for use as alternative gRNA promoters. The results from the first AAV transduction experiment are portrayed in the bar plots shown in FIGS. 86-87. These data demonstrate that use of AAV constructs containing the alternative U6 promoter isoforms resulted in similar or worse levels of editing compared to the editing level achieved when using AAV constructs containing the benchmark hU6 isoform 1 promoter. The results in FIGS. 86-87 further indicate that among the isoforms evaluated in this first experiment, use of the hU6 isoform 5 promoter may be the most promising and comparable to the benchmark hU6 isoform 1 promoter. Each vector also displayed dose-dependent editing at the target AAVS1 locus.

The results from the second AAV transduction experiment are portrayed in the bar plots shown in FIGS. 88-89. The data similarly demonstrate that use of AAV constructs containing the alternative U6 promoter isoforms resulted in similar or worse levels of editing compared to the editing level achieved when using AAV constructs with the benchmark hU6 isoform 1 promoter, recapitulating findings observed in FIGS. 86-87. The results continue to indicate the hU6 isoform 5 promoter as a comparable alternative to the benchmark hU6 isoform 1 promoter. The results also suggest use of the following U6 isoforms as comparable alternatives given that the editing levels achieved were comparable to that attained for the benchmark promoter: hU6 isoform 2 and its CpG-reduced forms (CpG-depleted hU6 isoform 2 and CpG-reduced hU6 isoform 2), and hU6 isoform 4 (FIGS. 88-89).

The results from the third AAV transduction experiment are portrayed in the bar plots shown in FIG. 90. The data similarly show that use of AAV constructs containing the indicated alternative U6 promoter isoforms resulted in comparable or slightly worse levels of editing as that achieved by the benchmark hU6 isoform 1 promoter. The results continue to support hU6 isoform 2 as a promising alternative Pol III promoter; the data also further show that use of the mU6 promoter as a comparable alternative Pol III promoter (FIG. 90).

These experiments demonstrate that alternative gRNA promoters, such as the various U6 isoforms evaluated in this example (in addition to the promoters identified and tested in Example 5), can be used to drive expression of the gRNA. Use of these alternative gRNA promoters would help reduce recombination risk during AAV production and packaging (especially if utilized in the context of a dual-guide AAV construct), while also modulate the resulting editing activity and potency of the Cas:gRNA system by differentially regulating the activity of the gRNAs. Furthermore, the identification and use of CpG-depleted U6 isoform promoters as alternative gRNA promoters would help mitigate potential undesired immune activation and enable therapeutic efficacy.

### Example 32: Assessment of CpG-Depleted CasX 515 Variants on CasX-Mediated Editing

As discussed in Example 18, unmethylated CpG motifs act as PAMPs (pathogen associated molecular patterns) that potently trigger undesired immune activation. Therefore, experiments were performed to deplete CpG motifs in the AAV construct encoding for CasX protein 515 and demonstrate that these CpG-depleted CasX 515 variants can edit effectively in vitro.

### Materials and Methods

### Design of CpG-Depleted and Codon-Optimized CasX 515 Variants and AAV Plasmid Cloning:

Nucleotide substitutions to replace native CpG motifs in CasX protein 515, as well as the flanking c-MYC NLSes, were rationally designed with codon optimization using various publicly available algorithms. As a result, the amino acid sequence of the encoding sequence of CpG-depleted CasX 515 with flanking c-MYC NLSes would be the same as the amino acid sequence of the corresponding encoding sequence of native CasX 515 with flanking c-MYC NLSes. Table 54 provides the sequences of CpG-depleted and codon-optimized variants of CasX 515 with flanking c-MYC NLSes, as well as the corresponding non-CpG depleted CasX 515 with flanking c-MYC NLSes.

TABLE 54
Sequences of CpG-depleted and codon-optimized
variants of CasX 515 with flanking c-MYC NLSes.

| CpG-depleted or non-CpG-depleted | | |
|---|---|---|
| variant 515 with flanking c-MYC NLSes | AAV | SEQ |
| (version no.) | construct ID | ID NO |
| CpG-depleted 515 (v1) | 290 | 9369 |
| CpG-depleted 515 (v2) | 291 | 9370 |
| CpG-depleted 515 (v3) | 292 | 9371 |
| CpG-depleted 515 (v4) | 293 | 9372 |
| CpG-depleted 515 (v5) | 294 | 9373 |
| CpG-depleted 515 (v6) | 295 | 9374 |
| CpG-depleted 515 (v7) | 296 | 9375 |
| CpG-depleted 515 (v8) | — | 9376 |
| CpG-depleted 515 (v9) | — | 9377 |
| CpG-depleted 515 (v10) | — | 9378 |
| CpG-depleted 515 (v11) | 297 | 9379 |
| CpG-depleted 515 (v12) | — | 9380 |
| Non-CpG-depleted 515 | 298 | 9381 |

All resulting sequences of the CpG-depleted and codon-optimized variants of CasX 515 with flanking c-MYC NLSes were cloned into a base AAV plasmid (sequences shown in Table 55). gRNA scaffold 235 and spacer 31.63, which targets the AAVS1 locus, were used for the experiments discussed in this example. The resulting AAV constructs were generated using standard molecular cloning techniques. Cloned and sequence-validated plasmid constructs were midi-prepped for subsequent nucleofection and AAV vector production.

TABLE 55
Sequences encoding for a base AAV plasmid into which CpG-depleted variants
of CasX 515 in Table 54 were cloned.

| | | DNA Sequence | Length of |
|---|---|---|---|
| AAV construct ID | Component Name | or SEQ ID NO | Component (bp) |
| 290 through 298 | 5′ ITR | 17 | 130 |
| | buffer sequence | 3684 | 23 |
| | UIA promoter | 3722 | 252 |
| | buffer sequence | 9302 | 18 |
| | Kozak | GCCACC | 6 |
| | start codon + CpG- | See sequences | |
| | depleted (or non- | listed in | |
| | CpG-depleted) & | Table 54 | |
| | codon-optimized | | |
| | CasX 515 with | | |
| | flanking c-MYC | | |
| | NLSes | | |
| | stop codon | TGA | 3 |
| | buffer sequence | 9366 | 30 |
| | bGH poly(A) | 3401 | 208 |
| | buffer sequence | GGTACCGT | 8 |
| | U6 promoter | 3563 | 241 |
| | buffer sequence | GAAACACC | 8 |
| | Scaffold 235 | 3638 | 99 |
| | AAVS1 spacer | 9354 | 20 |
| | (31.63) | | |
| | buffer sequence | 3700 | 17 |
| | 3′ ITR | 3701 | 141 |

### Transfection of HEK293 Cells In Vitro:

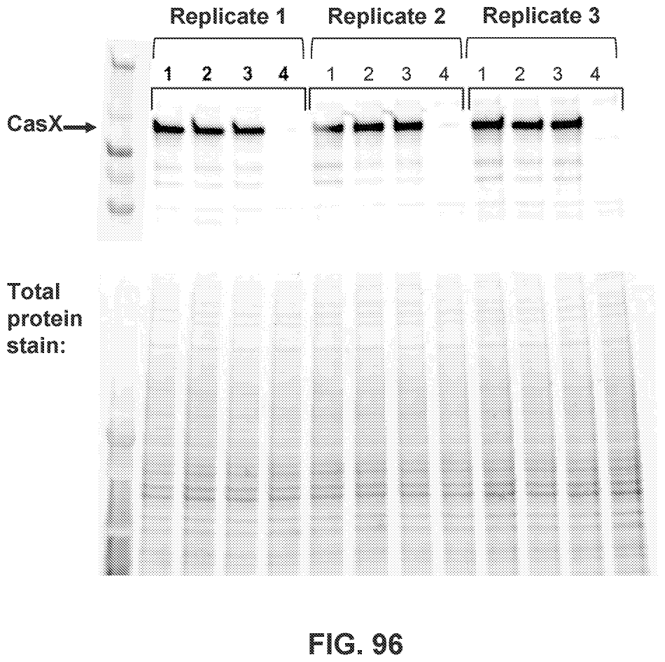

˜50,000 HEK293 cells per well were seeded on 24-well plates; two days later, cells were transfected with AAV plasmids containing sequences for a non-CpG-depleted (CpG⁺) CasX 515 (Table 54) or a version 1 of a CpG-depleted and codon-optimized CasX 515 variant (CpG⁻ vi CasX 515; SEQ ID NO: 9369, Table 54) following standard methods using lipofectamine. Two days later, cells were harvested to extract total protein lysate for western blotting analysis. Quantification of protein concentration and western blotting were performed using standard procedures. Three technical replicates were performed (Replicates 1-3) for the western blot. The results of this experiment are shown in FIG. 96. Untransfected cells served as an experimental control.

### AAV Production and Titering:

AAV production was performed using similar methods as described in Example 18. AAV titering was performed by ddPCR using a primer-probe set specific to bGH, an indicator of the AAV transgene, following similar methods as described in Example 31.

### AAV Transduction of iNs (Induced Neurons) In Vitro:

For one experiment, ˜30,000 iNs per well were seeded Matrigel-coated 96-well plates 7 days prior to transduction. Cells were transduced with AAVs expressing the CasX:gRNA system, a non-CpG-depleted CasX 515 (CpG⁺; Table 54) or version 1 of the CpG-depleted and codon-optimized CasX 515 variant (CpG⁻ v1; Table 54) and codon-optimized variants of CasX 515, at an MOI of 1E4 vg/cell. 7 days post-transduction, cells were harvested for gDNA extraction for editing analysis at the AAVS1 locus using NGS following similar methods as described in Example 18. One replicate was performed this experiment, and the results are shown in Table 56.

### AAV Transduction of HEK293 Cells In Vitro:

In a second experiment, ˜5,000 HEK293 cells per well are seeded on 96-well plates two days prior to transduction. AAVs expressing the CasX:gRNA system, containing various CpG-depleted and codon-optimized variants of CasX 515, are diluted in neuronal plating media and added to cells. Cells are transduced at four MOIs (1E4, 3E3, 1E3, or 3.7E2 vg/cell). Five days post-transduction, cells are harvested for gDNA extraction for editing analysis at the AAVS1 locus using NGS following similar methods as described in Example 18.

### Results

In one experiment, HEK293 cells were transiently transfected with AAV plasmids containing a CpG⁺ CasX 515 sequence or CpG⁻ v1 CasX 515 sequence. Four days post-transfection, CasX expression and editing activity at the AAVS1 locus were evaluated by western blotting and NGS respectively. The results of the western blotting analysis are portrayed in FIG. 96, showing CasX protein levels in transfected HEK293 cells, with a total protein stain blot (bottom blot) serving as the loading control. Cells transfected with the AAV plasmid containing a CpG⁺ CasX 515 sequence are labeled as “CpG⁺ CasX 515” (lane 1), while cells transfected with the construct harboring a CpG⁻ CasX 515 sequence are labelled as “CpG⁻ CasX 515_A” (lane 2) and “CpG⁻ CasX 515_B” (lane 3). Untransfected HEK293 cells are labelled “No plasmid control” (lane 4). The results in FIG. 96 show that expressing the AAV plasmid containing either the CpG⁻ or CpG⁺ CasX 515 sequence resulted in CasX expression. Editing activity at the AAVS1 locus was also assessed in human iNs; the results show that use of the AAV plasmid with either CpG⁻ v1 or CpG⁺ CasX 515 sequence resulting in editing at the target locus (Table 56).

TABLE 56
Results of the editing assay at the AAVS1
locus when using AAV plasmid containing either
CpG⁻ or CpG⁺ CasX 515.

| Experimental condition | Indel rate at AAVS1 locus |
|---|---|
| AAV plasmid with CpG⁺ 515 | 20.82% |
| AAV plasmid with CpG⁻ 515 (v1) | 16.55% |
| ‘No plasmid’ control | 0.06% |

The experiments demonstrate that depleting CpG motifs in the AAV construct encoding for CasX protein 515 resulted in sufficient CasX expression to induce effective editing at the target locus in vitro. Incorporating CpG-depleted AAV elements into the AAV genome would potentially reduce the risk of immunogenicity post-delivery of AAVs into target cells and tissues.

### Example 33: Additional Assessment of the Effects of Using CpG-Reduced or Depleted gRNA Scaffolds on CasX-Mediated Editing Activity

As discussed in Example 18, unmethylated CpG motifs act as PAMPs that potently trigger undesired immune activation; therefore, nucleotide substitutions to replace native CpG motifs in the AAV constructs, including that encoding for guide scaffold variants 235 and 316, were designed and generated. Here, experiments were performed to evaluate further the effects of using these resulting CpG-reduced or depleted gRNA scaffolds on CasX-mediated editing activity.

### Materials and Methods

The CpG-reduced or depleted scaffolds 320-341 were evaluated in three in vitro experiments described below; the sequences of scaffolds 320-341 are listed in Table 38. In addition, two newly engineered gRNA scaffolds, scaffold 382 and 392 (sequences listed in Table 57), were also assessed. As benchmark comparisons, scaffold 174, 235, and 316 (sequences listed in Table 37 and Table 57) were also included for evaluation.

TABLE 57
Sequences of additional gRNA scaffolds tested in this example.

| Scaffold ID | DNA SEQ ID NO: | RNA SEQ ID NO: |
|---|---|---|
| Scaffold 382 | 9355 | 9357 |
| Scaffold 392 | 9356 | 9358 |
| Scaffold 174 | 3631 | 2238 |

AAV constructs were designed and generated as previously described in Example 18. The CpG-reduced or depleted gRNA scaffolds were tested in two different AAV backbones. Specifically, for the experiment involving lipofection of HEK293 cells as described below, scaffolds 235 and 320-341 were tested in AAV vectors that were CpG-depleted, with the exception of AAV2 ITRs, as previously described in Example 18. Briefly, the CpG-depleted AAV backbone construct encoded for CpG-depleted versions of the following elements: U1A promoter, CasX 491, bGH poly(A) signal sequence, and U6 promoter. For the experiment involving AAV transduction of human induced neurons (iNs) and HEK293 cells as described below, scaffolds 174, 235, 316, 320-341, 382, and 392 were tested in an AAV backbone that was not CpG-depleted (see Table 58 for sequences). Furthermore, spacer 7.37 targeting the B2M locus was used in two experiments described below involving HEK293 cells: lipofection and AAV transduction. Spacer 31.63 targeting the AAVS1 locus was used in an experiment described below involving human iNs. Table 59 below lists the AAV constructs that were tested in the context of a non-CpG-depleted AAV vector and the experimental conditions in which these constructs were assessed.

TABLE 58
Sequences encoding for a base AAV plasmid into which gRNA scaffolds in Table
57 were cloned.

| | | Length of |
|---|---|---|
| Component Name | DNA sequence or SEQ ID | Component (bp) |
| 5′ ITR | 17 | 130 |
| buffer sequence | 3684 | 23 |
| U1A promoter | 3722 | 252 |
| buffer sequence | 9302 | 18 |
| Kozak | GCCACC | 6 |
| start codon + c-MYC NLS | 9359 | 33 |
| linker | TCTAGA | 6 |
| CasX 515 | 9352 | 2934 |
| linker | GGATCC | 6 |
| c-MYC NLS | 9353 | 27 |
| stop codon | TAA | 3 |
| buffer sequence | 3695 | 30 |
| bGH poly(A) | 3401 | 208 |
| buffer sequence | GGTACCGT | 8 |
| U6 promoter | 3563 | 241 |
| buffer sequence | GAAACACC | 8 |
| Scaffold variants | See sequences listed in | — |
| | Tables 37, 38, and 57 | |
| B2M spacer (spacer 7.37) | 9360 | 20 |
| AAVS1 spacer (spacer | 9354 | 20 |
| 31.63) | | |
| Non-targeting spacer | 9318 | 20 |
| (spacer 0.0) | | |
| buffer sequence | 3700 | 17 |
| 3′ ITR | 3701 | 141 |

TABLE 59
List of AAV constructs and scaffold variants tested in a non-CpG-
depleted AAV vector (see Table 58 for sequences) and the experimental
conditions in which these constructs were assessed.

| AAV | Scaffold | | |
|---|---|---|---|
| construct ID | variant | Spacer | Experimental conditions |
| 262 | 235 | 31.63 | AAV transduction in iNs |
| 263 | 328 | 31.63 | AAV transduction in iNs |
| 264 | 329 | 31.63 | AAV transduction in iNs |
| 265 | 382 | 31.63 | AAV transduction in iNs |
| 266 | 174 | 31.63 | AAV transduction in iNs |
| 267 | 335 | 31.63 | AAV transduction in iNs |
| 268 | 325 | 31.63 | AAV transduction in iNs |
| 269 | 330 | 31.63 | AAV transduction in iNs |
| 270 | 327 | 31.63 | AAV transduction in iNs |
| 271 | 334 | 31.63 | AAV transduction in iNs |
| 272 | 339 | 31.63 | AAV transduction in iNs |
| 273 | 337 | 31.63 | AAV transduction in iNs |
| 274 | 235 | Non-targeting | AAV transduction in iNs |
| 275 | 331 | 7.37 | AAV transduction in HEK293s |
| 276 | 335 | 7.37 | AAV transduction in HEK293s |
| 277 | 316 | 7.37 | AAV transduction in HEK293s |
| 278 | 392 | 7.37 | AAV transduction in HEK293s |
| 279 | 325 | 7.37 | AAV transduction in HEK293s |
| 280 | 334 | 7.37 | AAV transduction in HEK293s |
| 281 | 324 | 7.37 | AAV transduction in HEK293s |
| 282 | 336 | 7.37 | AAV transduction in HEK293s |
| 283 | 330 | 7.37 | AAV transduction in HEK293s |
| 284 | 320 | 7.37 | AAV transduction in HEK293s |
| 285 | 332 | 7.37 | AAV transduction in HEK293s |
| 286 | 321 | 7.37 | AAV transduction in HEK293s |
| 287 | 339 | 7.37 | AAV transduction in HEK293s |
| 288 | 235 | 7.37 | AAV transduction in HEK293s |
| 289 | 235 | Non-targeting | AAV transduction in HEK293s |

AAV production was performed using similar methods described in Example 18. For the experiment involving lipofection of HEK293 cells as described below, AAV titering was performed following similar methods as described in Example 1. For the two experiments involving AAV transduction of human iNs or HEK293 cells as described below, AAV titering was performed by ddPCR following similar methods as described in Example 31.

Cell-Based Assays Evaluating the Effects of Using CpG-Depleted or Reduced gRNA Scaffolds on Editing Activity:

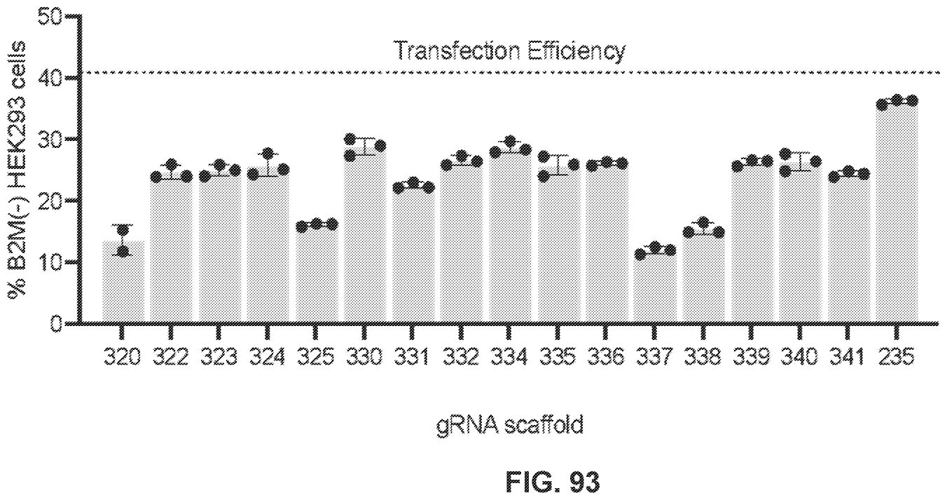

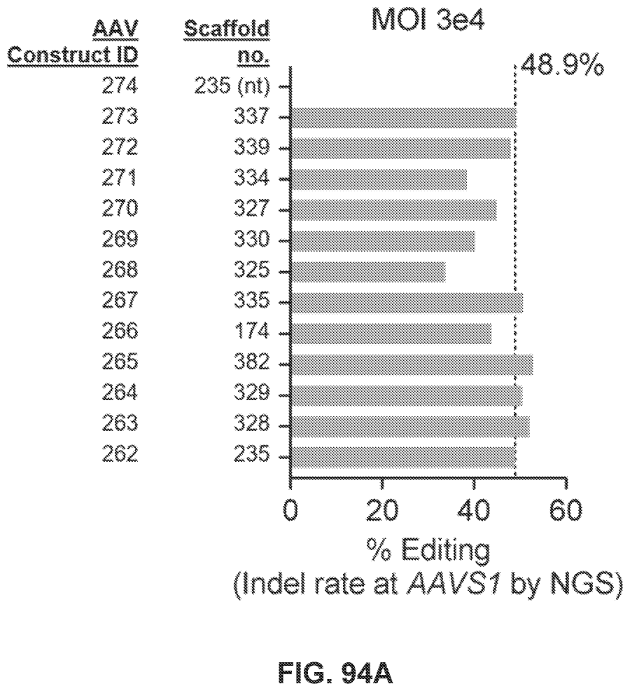

In one experiment, ˜20,000 HEK293 cells per well were seeded in 96-well plates 24 hours prior to transfection. Seeded cells were then transfected with CpG-depleted AAV plasmids containing various versions of the guide scaffold (scaffolds 320-341). 5 days post transfection, cells were harvested for B2M protein expression analysis via HLA immunostaining following by flow cytometry, following methods as described in Example 18. A CpG-depleted AAV plasmid with scaffold variant 235 served as an experimental control. An AAV plasmid with a CMV promoter driving mCherry expression was used as a transfection control, and a ˜41% transfection rate was observed. The results from this experiment are shown in FIG. 93.

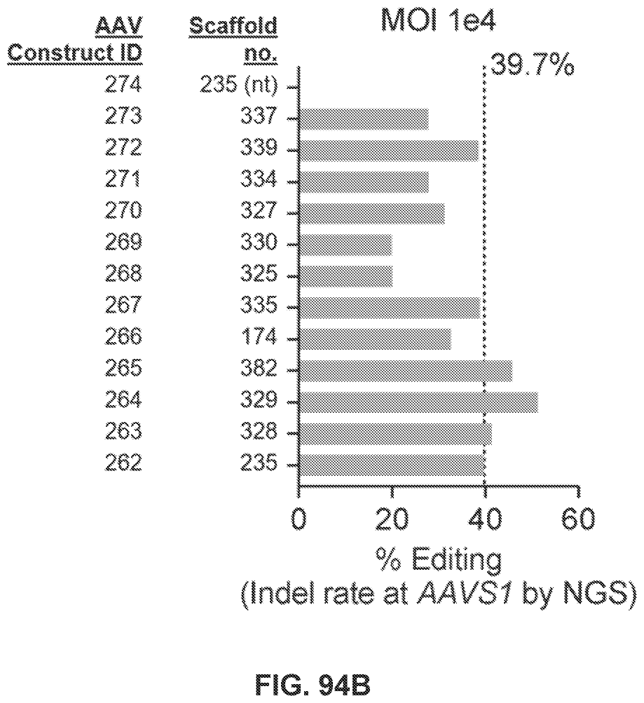

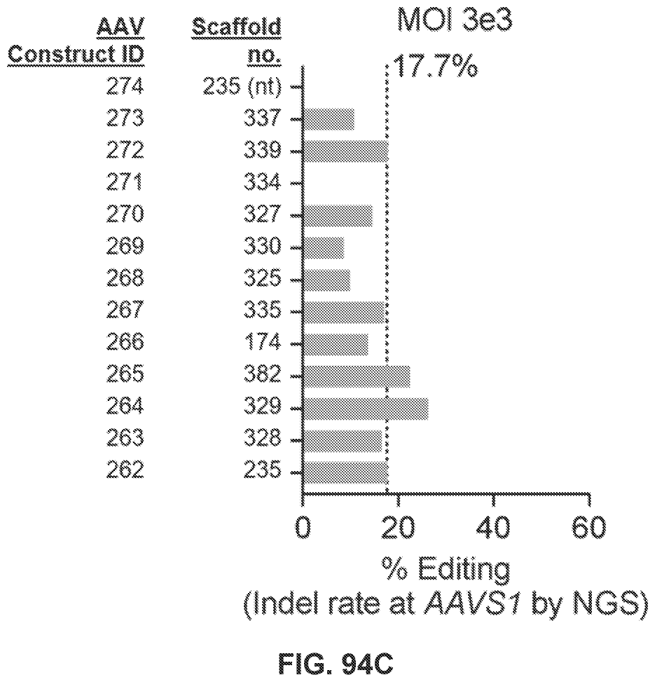

In a second experiment, ˜20,000 iNs per well were seeded on Matrigel-coated 96-well plates 7 days prior to transduction. AAVs expressing the CasX:gRNA system, containing various versions of the guide scaffold (AAV construct ID #262-274; see Table 59), were diluted in neuronal plating media and added to cells 7 days post-plating. Cells were transduced at three MOIs (3E4, 1E4 or 3E3 vg/cell). 7 days post-transduction, cells were gDNA extraction for editing analysis at the AAVS1 locus using NGS following similar methods as described in Example 18. The results from this experiment are shown in FIGS. 94A-94C.

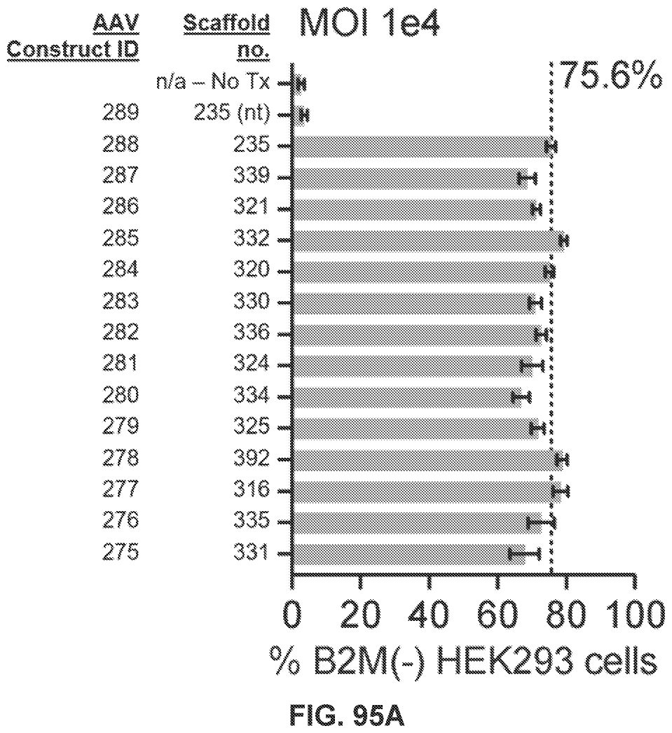

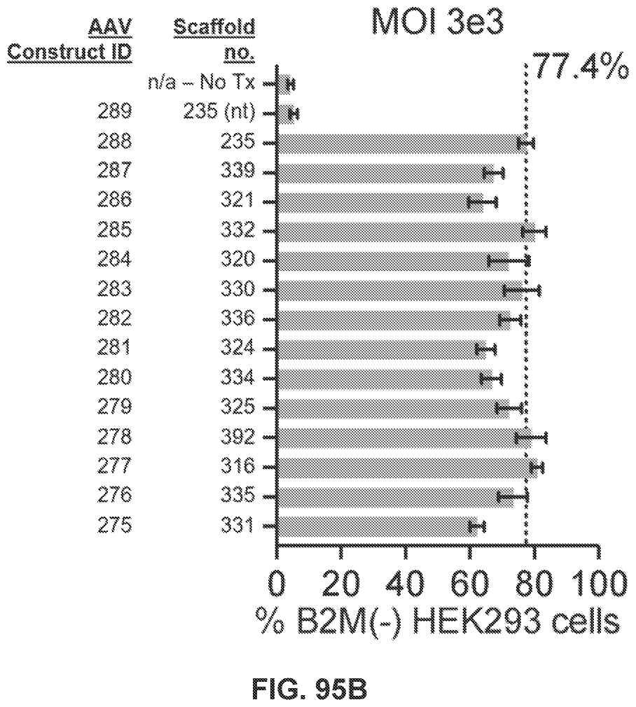

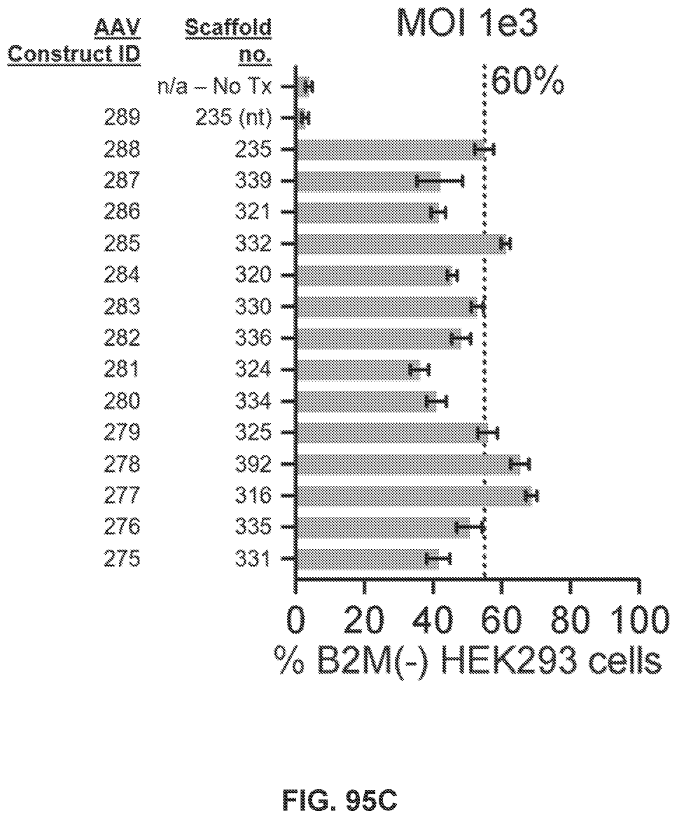

In a third experiment, ˜10,000 HEK293 cells per well were seeded in 96-well plates 24 hours prior to transfection. Seeded cells were then transduced with AAVs expressing the CasX:gRNA system, containing various versions of the guide scaffold (AAV construct ID #275-289; see Table 59). Cells were transduced at three MOIs (1E4, 3E3, or 1E3 vg/cell). 5 days post-transduction, cells were harvested for B2M protein expression analysis via HLA immunostaining following by flow cytometry, following methods as described in Example 18. The results from this experiment are shown in FIGS. 95A-95C.

### Results

Experiments were performed to evaluate further the effects of using CpG-reduced or depleted gRNA scaffolds on CasX-mediated editing activity. In the first experiment (N=1), HEK293 cells were lipofected with CpG-depleted AAV plasmids containing various versions of the gRNA scaffold (scaffolds 320-341). B2M protein expression was subsequently analyzed, and the results of the assay are shown in FIG. 93. The data demonstrate that use of scaffolds 320-341 did not improve editing activity at the target B2M locus, since use of these scaffolds produced a lower percentage of cells with B2M-relative to the level achieved when using an AAV construct containing scaffold 235. These results do not recapitulate the results observed in Example 18 (see FIGS. 77A-77B).

In the second experiment (N=1), human iNs were transduced with AAV particles expressing the CasX:gRNA system, containing various versions of the guide scaffold (AAV construct ID #262-274). Editing at the AAVS1 locus was analyzed, and the results of the assay are shown in FIGS. 94A-94C. The data demonstrate that of the scaffold variants tested, use of scaffold variant 329 and 382 appeared to improve editing at the AAVS1 locus when compared to use of scaffold 235, especially at MOI of 1E4 and 3E3 vg/cell. Furthermore, the effects on editing activity were observed in a dose-dependent manner.

In the third experiment (N=1), HEK293 cells were transduced with AAV particles expressing the CasX:gRNA system, containing various versions of the guide scaffold (AAV construct ID #275-289). B2M protein expression was subsequently analyzed, and the results of the assay are shown in FIGS. 95A-95C. The data demonstrate that of the scaffold variants tested, use of scaffolds 316, 392 and 332 appeared to improve editing at the B2M locus when compared to use of scaffold 235 overall. Specifically, at the higher MOI of 1E4 and 3E3 vg/cell, slightly improved editing was observed with use of scaffolds 316, 392, and 332 (FIGS. 95A-95B), while a stronger editing improvement was observed at the lower MOI of 1E3 vg/cell (FIG. 95C). Notably, scaffold 332 and 392 both include CG >GC mutations in the pseudoknot stem (region 1; FIGS. 76A-76B), effectively reducing the overall number of CpGs when compared to scaffold 235, thereby potentially contributing to the increase in editing activity. Furthermore, scaffolds 316 and 332 both have a truncated extended stem when compared to scaffold 235, removing the bubble and the CG dinucleotide (region 3; FIGS. 76A-76B), thereby also potentially contributing to the observed increase in editing activity. Further experiments are performed, especially at lower MOIs, to unravel the intricacies of the effects of individual CpG mutations on editing potency.

The results from the experiments described here demonstrate that use of guide scaffolds with different levels of CpG depletion can result in varying levels of editing mediated by the CasX:gRNA system, and that the resulting editing levels can vary by method of delivery (e.g., plasmid transfection vs. AAV transduction).

### Example 34: Assessment of Editing by Engineered Variants of CasX Nucleases at a Target Locus in Human Cells Transduced by AAVs

Experiments were performed to demonstrate that engineered variants of CasX nucleases were able to edit a target locus in human cells when delivered via AAV transduction.

### Materials and Methods

AAV plasmid constructs were generated and cloned into a pAAV plasmid flanked with AAV2 ITRs using standard molecular cloning methods as described in Example 1. Briefly, AAV plasmids were generated to express a CasX protein driven by the UbC promoter with a bGH poly(A) signal sequence and a gRNA transcriptional unit that expressed a Pol III promoter-guide scaffold 235 with spacer 31.63 targeting the AAVS1 locus. In this example, CasX variants 491, 515, 528, 572, 593, 672, 676, and 690 were assessed for their editing activity in human induced neurons (iNs). The sequences of the AAV constructs with the various CasX proteins and AAVS1-targeting gRNA are shown in Table 60.

TABLE 60
Sequences of AAV constructs with various CasX proteins assessed in this
example.

| | | DNA sequence or | Length of |
|---|---|---|---|
| AAV construct ID | Component Name | SEQ ID NO | Component (bp) |
| 245 through 252 | 5′ ITR | 17 | 130 |
| | buffer sequence | 3684 | 23 |
| | UbC promoter | 3533 | 400 |
| | buffer sequence | 9302 | 18 |
| | Kozak | GCCACC | 6 |
| | start codon + c-MYC | 9359 | 33 |
| | NLS | | |
| | linker | TCTAGA | 6 |
| 245 | CasX 491 | 9291 | 2931 |
| 246 | CasX 672 | 3710 | 2934 |
| 247 | CasX 676 | 9361 | 2937 |
| 248 | CasX 528 | 9362 | 2934 |
| 249 | CasX 690 | 9363 | 2934 |
| 250 | CasX 515 | 9352 | 2934 |
| 251 | CasX 572 | 9364 | 2934 |
| 252 | CasX 593 | 9365 | 2934 |
| 245 through 252 | linker | GGATCC | 6 |
| | c-MYC NLS | 9353 | 27 |
| | stop codon | TAA | 3 |
| | buffer sequence | 3695 | 30 |
| | bGH poly(A) | 3401 | 208 |
| | buffer sequence | GGTACCGT | 8 |
| | U6 promoter | 3563 | 241 |
| | buffer sequence | GAAACACC | 8 |
| | Scaffold 235 | 3638 | 99 |
| | AAVS1 spacer | 9354 | 20 |
| | (31.63) | | |
| | buffer sequence | 3700 | 17 |
| | 3′ ITR | 3701 | 141 |

* Components are listed in a 5′ to 3′ order within the constructs

AAV production and titering were performed following similar methods as described in Example 1.

### AAV Transduction of iNs (Induced Neurons) In Vitro:

˜50,000 iNs per well were seeded on Matrigel-coated 96-well plates 7 days prior to transduction. AAVs expressing the CasX:gRNA system, containing various CasX proteins (sequences in Table 60), were diluted in neuronal plating media and added to cells. Cells were transduced at three MOIs (3E3, 1E3 or 3E2 vg/cell). 7 days post-transduction, cells were lifted using lysis buffer, and gDNA was harvested and prepared for editing analysis at the AAVS1 locus using NGS following similar methods as described in Example 18.

### Results

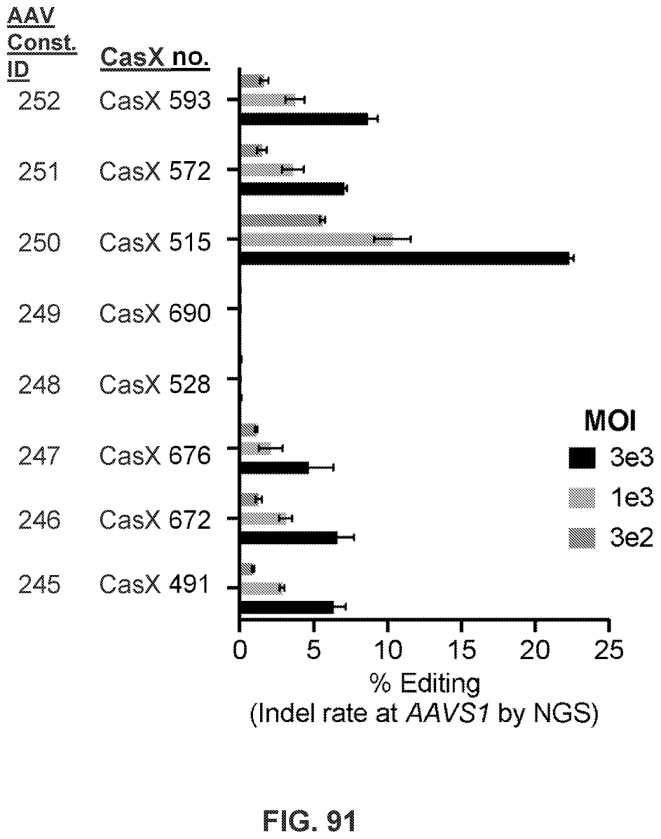

Human iNs were transduced with AAVs containing CasX proteins 491, 515, 528, 572, 593, 672, 676, or 690 and guide scaffold variant 235 with spacer 31.63 targeting the safe harbor AAVS1 locus to assess the editing activity of these various CasX proteins. The results of this editing assay are displayed in FIG. 101. The data demonstrate that of the CasX variants paired with the spacer 31.63, a spacer with the TTC motif, CasX 515 demonstrated the highest editing activity (≥20%) at the highest MOI of 3E3 vg/cell. Furthermore, as anticipated, ATC-specific variants CasX 528 and 690 were unable to edit at the AAVS1 locus when paired with the TTC-specific spacer 31.63 (FIG. 91). A dose-dependent response was also observed across all CasX variants that demonstrated editing activity.

The experiments demonstrate that engineered variants of CasX nucleases, when paired with the appropriate spacer, were able to edit a target locus in human cells when delivered via AAV transduction.

### Example 35: Assessment of Various Protein Promoters on CasX Protein Editing Activity

Experiments were performed to demonstrate that CasX protein editing activity and potency can be influenced by using different protein promoters in an AAV construct to drive expression of the encoded protein. Furthermore, the effects incorporating a WPRE element on editing activity were also evaluated.

### Materials and Methods

AAV plasmid constructs were generated and cloned into a pAAV plasmid flanked with AAV2 ITRs using standard molecular cloning methods as described in Example 1. Briefly, AAV plasmids were generated to express CasX 491 driven by a protein promoter variant with a bGH poly(A) signal sequence and a gRNA transcriptional unit that expressed a Pol III promoter-guide scaffold 235 with spacer 31.63 targeting the AAVS1 locus. Three protein promoters were assessed in this example: U1A, Jet, and UbC, which were cloned upstream of the construct encoding for CasX 491. Each protein promoter variant was also assessed with or without a WPRE element (WPRE2 or WPRE3). For constructs with a WPRE element, the WPRE element was cloned downstream of the construct encoding for CasX 491. Sequences for the three protein promoters and two WPRE elements are listed in Table 61. The specific combinations of protein promoter and WPRE elements with the corresponding AAV construct IDs are listed in Table 62.

TABLE 61
Sequences of protein promoter variants and
WPRE elements assessed in this example.

| SEQ | | AAV | Length of |
|---|---|---|---|
| ID NO | Element | construct ID | element (bp) |
| 3533 | UbC protein promoter | 253, 254, 255 | 400 |
| 3556 | Jet protein promoter | 256, 257, 258 | 164 |
| 3722 | U1A protein promoter | 259, 260, 261 | 252 |
| 3616 | WPRE2 | 253, 256, 259 | 593 |
| 3617 | WPRE3 | 254, 257, 260 | 247 |

TABLE 62
Combination of protein promoter and WPRE
elements assessed in this example.

| AAV construct ID | Protein promoter | WPRE element |
|---|---|---|
| 253 | UbC | WPRE2 |
| 254 | UbC | WPRE3 |
| 255 | UbC | N/A |
| 256 | Jet | WPRE2 |
| 257 | Jet | WPRE3 |
| 258 | Jet | N/A |
| 259 | U1A | WPRE2 |
| 260 | U1A | WPRE3 |
| 261 | U1A | N/A |

AAV production and titering were performed following similar methods as described in Example 1.

### AAV Transduction of iNs (Induced Neurons) In Vitro:

˜50,000 iNs per well were seeded on Matrigel-coated 96-well plates 24 hours prior to transduction. AAVs expressing the CasX:gRNA system, containing various protein promoter variants with or without a WPRE element, were diluted in neuronal plating media and added to cells. Cells were transduced at two MOIs (1E3 or 1E4 vg/cell). 7 days post-transduction, cells were lifted using lysis buffer, and gDNA was harvested and prepared for editing analysis at the AAVS1 locus using NGS following similar methods as described in Example 18.

### Results

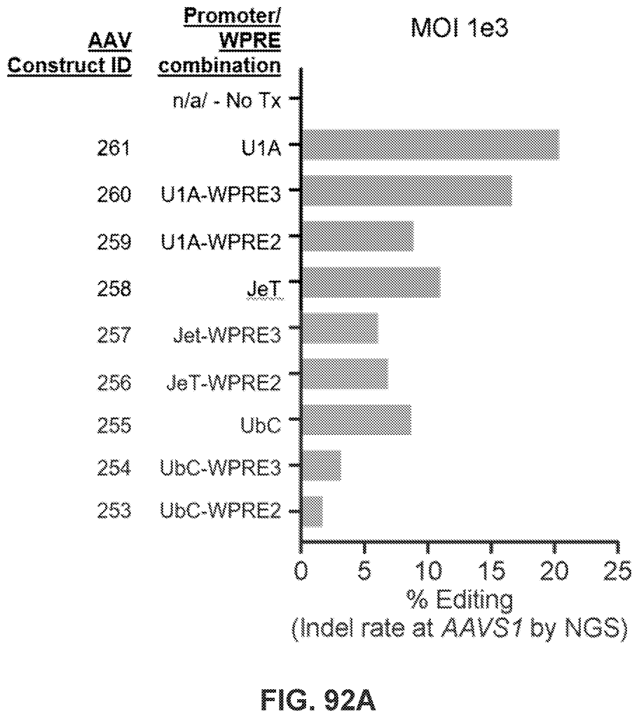

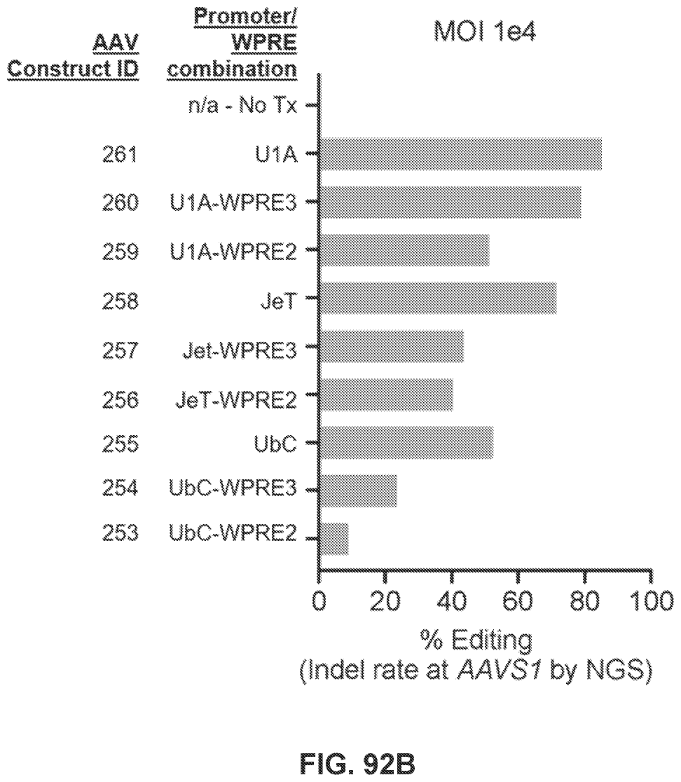

The effects of using different protein promoters (U1A, Jet, and UbC), with or without a WPRE element, on editing activity were evaluated. The results of this editing assay are shown in FIGS. 92A-92B. The data demonstrate that use of the U1A protein promoter resulted in the highest editing activity at the AAVS1 locus at both MOIs. Specifically, use of the U1A protein promoter to drive CasX expression resulted in >20% editing at the MOI of 1E3, and this editing level increased to >80% at the MOI of 1E4. Furthermore, incorporation of a WPRE appeared to result in reduced editing activity at the AAVS1 locus (FIGS. 92A-92B).

The experiments demonstrate that the editing activity of CasX proteins can be modulated by protein promoter choice; here, of the three protein promoters assessed, use of the U1A promoter resulted in the highest level of CasX editing activity.

### Example 36: CasX:gRNA In Vitro Cleavage Assays

Experiments were performed to assess in vitro DNA cleavage by CasX:gRNA ribonucleoproteins (RNPs).

### Materials and Methods

### Assembly of RNP

RNPs of either CasX variant 119 (SEQ ID NO: 124), CasX variant 491 (SEQ ID NO: 190), CasX variant 515 (SEQ ID NO: 197), or CasX variant 812 (SEQ ID NO: 484) were assembled with single guide RNAs (sgRNA) with scaffold 316 (SEQ ID NO: 9588) and one of two spacers, as described in detail below. Separately, RNPs of CasX variant 515 were assembled with sgRNA with either scaffold 2 (SEQ ID NO: 5), 174 (SEQ ID NO: 2238), 235 (SEQ ID NO: 2292), or 316 (SEQ ID NO: 9588) and one of two spacers.

Purified RNP of CasX variants and sgRNA were prepared same-day prior to experiments. For experiments where protein variants were being compared, the CasX protein was incubated with sgRNA at 1:1.2 molar ratio. When scaffolds were compared, the protein was added in 1.2:1 ratio to guide. Briefly, sgRNA was added to Buffer #1 (20 mM Tris HCl pH 7.5, 150 mM NaCl, 1 mM TCEP, 5% glycerol, 10 mM MgCl₂) on ice, then CasX was added to the sgRNA solution, slowly with swirling, and immediately incubated at 37° C. for 20 minutes to form RNP complexes. RNP complexes were centrifuged at 4° C. for 5 minutes at 16,000×g to remove any precipitate. Formation of competent (active) RNP was assessed as described below.

### In Vitro Cleavage Assays

The ability of CasX variants to form active RNP compared to a CasX variant 119 was determined using an in vitro cleavage assay. The beta-2 microglobulin (B2M) 7.9 and 7.37 target for the cleavage assay was created as follows. DNA oligos (sequences in Table 63) were generated with 5′ terminal amino modification for conjugation to Cy-dyes with an amino-reactive handle (N-hydroxysuccinimide). Oligo-dye conjugation reactions of 100 uM oligo and 1 mM dye were performed in 100 mM sodium borate pH 8.3 at 4° C. for 16 h. Target strands (TS) were labeled with Cy5.5 and non-targeting strands (NTS) were labeled with Cy7.5. After quenching the reactions with 1 mM Tris pH 7.5, the conjugated oligos were purified via ethanol precipitation. Double-stranded DNA (dsDNA) targets were formed by mixing the oligos in a 1:1 ratio in 1× hybridization buffer (20 mM Tris HCl pH 7.5, 100 mM KCl, 5 mM MgCl₂), heating to 95° C. for 10 minutes, and allowing the solution to cool to room temperature.

TABLE 63
DNA sequences and descriptions of target DNAs

| DNA Sequence (5′-3′)* | SEQ ID NO: | Description |
|---|---|---|
| /5AmMC6/TGAAGCTGACAGCATTCGGGCCGAGA | 9591 | 7.37 NTS with 5′ amine for NHS ester |
| TGTCTCGCTCCGTGGCCTTAGCTGTGCTCGCGCT | | linkages** |
| /5AmMC6/AGCGCGAGCACAGCTAAGGCCACGGA | 9592 | 7.37 TS with 5′ amine** |
| GCGAGACATCTCGGCCCGAATGCTGTCAGCTTCA | | |
| /5AmMC6/TGAAGCTGACAGCATTCGGGCCTAGA | 9593 | 7.37 gel probe NTS, mismatch at 5 |
| TGTCTCGCTCCGTGGCCTTAGCTGTGCTCGCGCT | | |
| /5AmMC6/AGCGCGAGCACAGCTAAGGCCACGGA | 9594 | 7.37 gel probe TS, mismatch at 5 |
| GCGAGACATCTAGGCCCGAATGCTGTCAGCTTCA | | |
| /5AmMC6/TGAAGCTGACAGCATTCGGGCCGAGA | 9595 | 7.37 gel probe NTS, mismatch at 10 |
| TATCTCGCTCCGTGGCCTTAGCTGTGCTCGCGCT | | |
| /5AmMC6/AGCGCGAGCACAGCTAAGGCCACGGA | 9596 | 7.37 gel probe TS, mismatch at 10 |
| GCGAGATATCTCGGCCCGAATGCTGTCAGCTTCA | | |
| /5AmMC6/TGAAGCTGACAGCATTCGGGCCGAGA | 9597 | 7.37 gel probe NTS, mismatch at 15 |
| TGTCTCGATCCGTGGCCTTAGCTGTGCTCGCGCT | | |
| /5AmMC6/AGCGCGAGCACAGCTAAGGCCACGGA | 9598 | 7.37 gel probe TS, mismatch at 15 |
| TCGAGACATCTCGGCCCGAATGCTGTCAGCTTCA | | |
| /5AmMC6/CTTTCAGCAAGGACTGGTCTTTCTAT | 9599 | 7.9 target TS |
| CTCTTGTACTACACTGAATTCACCCCCACTGAAA | | |
| /5AmMC6/TTTCAGTGGGGGTGAATTCAGTGTAG | 9600 | 7.9 target NTS |
| TACAAGAGATAGAAAGACCAGTCCTTGCTGAAAG | | |
| /5AmMC6/CTTTCAGCAAGGACTGGTCTTTCTAT | 9601 | 7.9 gel probe TS, mismatch at 5 |
| CTCTTGTACGACACTGAATTCACCCCCACTGAAA | | |
| /5AmMC6/TTTCAGTGGGGGTGAATTCAGTGTCG | 9602 | 7.9 gel probe NTS, mismatch at 5 |
| TACAAGAGATAGAAAGACCAGTCCTTGCTGAAAG | | |
| /5AmMC6/CTTTCAGCAAGGACTGGTCTTTCTAT | 9603 | 7.9 gel probe TS, mismatch at 10 |
| CTCTCGTACTACACTGAATTCACCCCCACTGAAA | | |
| /5AmMC6/TTTCAGTGGGGGTGAATTCAGTGTAG | 9604 | 7.9 gel probe NTS, mismatch at 10 |
| TACGAGAGATAGAAAGACCAGTCCTTGCTGAAAG | | |
| /5AmMC6/CTTTCAGCAAGGACTGGTCTTTCTGT | 9605 | 7.9 gel probe TS, mismatch at 15 |
| CTCTTGTACTACACTGAATTCACCCCCACTGAAA | | |
| /5AmMC6/TTTCAGTGGGGGTGAATTCAGTGTAG | 9606 | 7.9 gel probe NTS, mismatch at 15 |
| TACAAGAGACAGAAAGACCAGTCCTTGCTGAAAG | | |

*5AmMC6 indicates the 5′ Amino Modifier C6. The target sequences are underlined.
**The Kcleave assay using the mismatched position 5 dsDNA target was run at 37° C.

### Determining Cleavage-Competent Fractions for RNPs

Cleavage reactions were prepared with final RNP concentrations of 100 nM and final target concentration of 100 nM. Reactions were carried out at 37° C. and initiated by the addition of the dye-labeled dsDNA target. Aliquots were taken at 5, 30, and 60 minutes and quenched by adding to 9500 formamide, 25 mM EDTA. Samples were denatured by heating at 95° C. for 10 minutes and run on a 1000 urea-PAGE gel. The gels were imaged with a Cytiva Typhoon and quantified using the Cytiva IQTL software.

### Kcleave Assay

Cleavage reactions were set up with a final RNP concentration of 200 nM and a final target concentration of 10 nM. Reactions were carried out at 16° C., except where otherwise noted, and initiated by the addition of the target DNA. Aliquots were taken at 15, 30, 60, 120, 180, 240, and 480 seconds, and quenched by adding to 9500 formamide, 25 mM EDTA. Samples were denatured by heating at 95° C. for 10 minutes and run on a 10o urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

To test the relative specificities of engineered proteins in vitro, apparent cleavage rate constants were compared for targets with mismatched bases at various positions (5, 10, and 15 nt downstream of PAM, Table 63). Cleavage assays were performed in large excess of RNP (200 nM RNP and 1 nM target dsDNA) at 16° C., with the exception of assays measuring cleavage of the target with a mismatch at 5 nt, which were conducted at 37° C. in order to observe measurable cleavage rates. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

### Results

### Determining Cleavage-Competent Fractions for Protein Variants Compared to CasX Variant 119

To determine the cleavage-competent fraction for the tested CasX variants, it was assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. Thus, the active (competent) fraction for each RNP was derived from the cleaved fraction over the total signal at the 60-minute timepoint, upon confirming an increase in cleaved fraction from the 5-minute timepoint, and relative plateau in cleaved fraction from the 30-minute timepoint.

Apparent competent fractions were determined for the RNPs with various CasX variants and are provided in Table 64.

TABLE 64
Protein variant RNP comparison of fraction competence and Kcleave rates

| | | Kcleave (sec⁻¹) | | | |
|---|---|---|---|---|---|
| | | On- | Position 15 | Position 10 | Position 5 |
| RNP | Fraction competence* | target | mismatch | mismatch | mismatch |
| (CasX.Scaffold.Spacer) | (mean ± stdev) | spacer | spacer | spacer | spacer** |
| 119.174.7.9 | 26.71 ± 9.65% | 0.0292 | 0.0158 | 0.0047 | 0.0127 |
| 491.174.7.9 | 49.23 ± 6.37% | 0.0966 | 0.0552 | 0.03 | 0.0616 |
| 515.174.7.9 | 30.15 ± 3.58% | 0.0782 | 0.0625 | 0.0324 | 0.0392 |
| 812.174.7.9 | 53.28 ± 5.14% | 0.0909 | 0.0512 | 0.0142 | 0.01 |
| 119.174.7.37 | 29.01 ± 2.67% | 0.0022 | 0.0061 | 0.0028 | 0.0034 |
| 491.174.7.37 | 48.73 ± 5.44% | 0.0425 | 0.015 | 0.0297 | 0.0301 |
| 515.174.7.37 | 43.54 ± 16.19% | 0.0396 | 0.0127 | 0.0297 | 0.0347 |
| 812.174.7.37 | 38.23 ± 2.49% | 0.027 | 0.0111 | 0.0119 | 0.014 |

*Active fraction was calculated by averaging three experimental replicates.
**The Kcleave assay using the mismatched position 5 dsDNA target was run at 37° C.

For protein variant comparison, the following CasX variants were used with guide scaffold 316 and spacer 7.9 or guide 316 and spacer 7.37: CasX variant 119, CasX variant 491, CasX variant 515, and CasX variant 812. CasX variant 119 had the lowest active fraction for both spacers, indicating that CasX variant 491, CasX variant 515, and CasX variant 812 form more active and stable RNP with the identical guides under the tested conditions as compared to CasX variant 119. CasX variants 491, 515, and 812 did not show consistent trends in their competent fractions across the two spacers, consistent with the expectation that the additional engineering following CasX 491 primarily affects target engagement and cleavage, rather than guide binding or stability.

Kcleave Assay to Understand Specificity of RNPs Formed from CasX Variants

Assays were performed to measure the apparent first-order rate constant of non-target strand cleavage (k_{cleave}), and the results are presented in Table 64, above. A drastic effect on the kinetics of CasX variant 812 RNP cleavage was observed for on-target versus the mismatched dsDNA target for both spacers. CasX variant 812 had comparable on-target cleavage rates to CasX variant 491 and CasX variant 515 for both spacers, with a slightly higher cleavage rate than CasX variant 515 on spacer 7.9, which might be explained by the lower competent fraction observed for the CasX variant 515 RNP with that spacer, and a lower cleavage rate on 7.37.

The off-target rates for CasX variant 812 were much more substantially reduced for most of the mismatched substrates. The difference in kcleave rates was readily apparent for the target with a mismatch at position 10, with 812 having a roughly 6-fold (7.9) and 2-fold (7.37) reduction in cleavage rate, as compared to its on-target rate. CasX variant 515, by comparison, exhibited a 2.4-fold and a 25% reduction on the same targets. A substantial difference was also observed for the position 5 mismatch targets. Even though the assay was run at 37° C. to enable measurable cleavage rates, as the position 5 mismatch targets were essentially uncleaved by the CasX RNPs at the lower temperature used for the other targets, CasX variant 812 against spacer 7.9 exhibited a 9-fold reduction in cleavage rate from on-target rate run at 16° C. and a 2-fold reduction for the 7.37 spacer with a position 5 mismatch. CasX variant 515 showed a 2-fold reduction for mismatched 7.9 and a nearly equivalent cleavage rate for 7.37 with the position 5 mismatch (note that the “equivalent” cleavage rate is due to the increased temperature).

For the position 15 mismatch substrate, CasX variant 812 exhibited modest reductions in cleavage rates relative to on-target rates, comparable to the reduction observed for 515. This suggests that the increased sensitivity of CasX variant 812 to mismatches declines by the PAM distal region, at least for the specific mismatches and spacers tested here. The increased sensitivity at positions 5 and 10 in particular correlates with the position of the G329K mutation present in CasX variant 812. This mutation introduces a positive charge near the RNA spacer around position 8 and may help CasX to better read out distortions caused by mismatches. Mismatches closer to this new site of contact would be more likely to significantly disrupt either R-loop propagation or allosteric activation of the RuvC (depending on the precise mechanism of increased specificity), while mismatches farther away (as in the position 15 mismatch) might have more variable effects depending on the nature of the mismatch and its effects on the broader heteroduplex structure. Taken together, these data confirm that CasX variant 812 is inherently more sensitive to mismatches between the RNA spacer and the DNA target and is not simply a less active enzyme, as the decrease in cleavage rate at mismatched targets is in excess of the decrease in cleavage rate at properly matched targets. This is consistent with the results in Examples 37 and 38 that indicate that CasX variant 812 is a highly specific enzyme, with lower off-target editing compared to the other nucleases tested.

### Determining Cleavage-Competent Fractions for Single Guide Variants Relative to Reference Single Guide 2

RNPs were complexed using the aforementioned methods. To isolate the effect of sgRNA identity on RNP formation, guide-limiting conditions were employed. sgRNAs with scaffolds 2, 174, 235, or 316 with spacers 7.9 or 7.37 were mixed with CasX variant 515 at final concentrations of 1 μM for the guide and 1.2 μM for the protein. Fraction competence was calculated as described above, and the results are provided in Table 65.

TABLE 65
Guide variant RNP comparison of fraction
competence and Kcleave assay

| RNP construct | Competent fraction | Kcleave |
|---|---|---|
| (CasX.Scaffold.Spacer) | (mean ± stdev) | (sec⁻¹) |
| 515.2.7.9 | 28.146.87% | 0.1346 ± 0.0118 |
| 515.174.7.9 | 42.779.62% | 0.1723 ± 0.0046 |
| 515.235.7.9 | 34.111.15% | 0.1696 ± 0.0571 |
| 515.316.7.9 | 30.896.87% | 0.1413 ± 0.0301 |
| 515.2.7.37 | 10.422.24% | 0.0204 ± 0.0002 |
| 515.174.7.37 | 19.962.88% | 0.0534 ± 0.0200 |
| 515.235.7.37 | 32.6411.60% | 0.0647 ± 0.0163 |
| 515.316.7.37 | 26.9811.08% | 0.0851 ± 0.0071 |

* active fraction was calculated by averaging two experimental replicates

Given the complex folding structure of the CasX guide, fraction competence is expected to largely be determined by how much of the guide is properly folded for interaction with the protein. All guides with engineered scaffolds showed improvements over scaffold 2, but guides with scaffold 235 or 316 showed improvements relative to 174 for spacer 7.37. This is consistent with the introduction of mutations in the pseudoknot and triplex that are expected to stabilize the properly folded form.

Higher competent fractions of all guides were observed for spacer 7.9. For this spacer, scaffold 174 had the highest competent fraction, followed by scaffolds 316, 235, and 2. Proper guide folding is expected to be highly dependent on the potential for undesired interactions between the scaffold and spacer sequences, so the observed differences may be attributable to differential sequence-specific interactions, variations in prep quality, or noise in the assay.

Determining k_{Cleave} for Single Guide Variants Compared to Reference Scaffold 2

Cleavage assays were performed with CasX variant 515 and guides with reference scaffold 2 compared to guides with scaffolds 174, 235, or 316 with spacer 7.9 or 7.37 to determine relative cleavage rates. The mean and standard deviation of three replicates with independent fits are presented in Table 65, above.

To reduce cleavage kinetics to a range measurable with the assay, the cleavage reactions were incubated at 16° C. Under these conditions, all guides supported faster cleavage rates as compared to scaffold 2. For spacer 7.37, the cleavage kinetics aligned with those guides that contributed to the highest fraction competence, with the highest cleavage rate being sg174 (0.1723 s⁻¹), followed by scaffold 235 (0.1696 s⁻¹) and scaffold 316 (0.1413 s⁻¹), versus scaffold 2 (0.1346 s⁻¹). For spacer 7.9, scaffold 316 yielded the highest cleavage rate (0.0851 s⁻¹), followed by scaffold 235 (0.0647 s⁻¹) and sg174 (0.0534 s⁻¹), versus scaffold 2 (0.0204 s⁻¹). The fraction competence and k_{cleave} data did not demonstrate differences across the engineered variants that were consistent across both spacers, although all are consistently better than scaffold 2. This suggests that the improvements seen for scaffold 235 and 316 over 174 are primarily due to behavior in the cell, whether it be stability in the cytoplasm, folding in the cytoplasm, transcription when delivered via plasmid or AAV, or refolding ability when delivered via LNP, that are not captured by guides that have been in vitro transcribed, refolded, and tested for cleavage biochemically.

### Example 37: Identification of CasX Variants with Enhanced Activity or Specificity Relative to CasX Variant 515

An experiment was performed to identify CasX variants with single mutations and increased editing activity or improved specificity relative to CasX variant 515.

### Materials and Methods

A multiplexed pooled approach was taken to assay clonal proteins derived from CasX variant 515 using a pooled activity and specificity (PASS) assay. A pooled HEK cell line, which was adapted to suspension culture from adherent cells, was generated and termed PASS_V1.03. Methods to complete the production of the PASS_V1.03 line were previously described in International Publication No. WO2022120095A1, incorporated herein by reference.

CasX variants were expressed using a relatively weakly-expressing promoter to reduce CasX protein expression and thereby improve the sensitivity of the assay. Samples were tested in quadruplicate. The list of CasX variants tested and their mutations relative to CasX variant 515 is provided in Tables 66 and 67, below. All of the tested CasX variants had single mutations (i.e., a single amino acid substitution, deletion, or insertion) relative to CasX variant 515, except for CasX variant 676, which has three mutations relative to CasX variant 515. Streptococcus pyogenes Cas9 without a guide RNA served as a negative control.

To assess the editing activity and specificity of the tested CasX variants at human target sites, two sets of target sites were quantified. First, editing was quantified at TTC PAM on-target sites in which the twenty nucleotides of each gRNA spacer targeting these on-target sites were perfectly complementary to the target site. For each sample and spacer-target pair, data based on <500 reads were removed. Fraction indel values for each sample and spacer-target pair were subtracted by the average fraction indel value across Cas9-treated samples with the same spacer-target pair; Cas9 served as a negative control due to the absence of a compatible guide RNA. Second, editing was quantified at TTC PAM off-target sites, in which one of the twenty nucleotides of the spacer was mismatched with the target site. As above, for each sample and spacer-target pair, data based on <500 reads were removed, and fraction indel values for each sample and spacer-target pair were subtracted by the average fraction indel value across Cas9-treated samples with the same spacer-target pair. Finally, for those TTC PAM spacer-target pairs that had both an on-target and an off-target version, the average editing activity and standard error of the mean (SEM) were calculated.

### Results

Table 66 provides the level of on-target editing produced by various CasX variants with mutations relative to CasX variant 515, ranked from highest to lowest activity.

TABLE 66
Average on-targeting editing activity, ranked from highest to lowest

| Protein Designation | Mutation relative to | Average on-target TTC | SEM on-target TTC |
|---|---|---|---|
| (CasX variant no., | CasX 515* | PAM editing activity | PAM editing activity |
| or Cas9) | (Position.Reference.Alternative) | (fraction) | (fraction) |
| 607 | 398.Y.T | 2.72E−01 | 4.08E−02 |
| 532 | 27.—.R | 2.59E−01 | 3.34E−02 |
| 676 | 27.—.R & 170.L.K & | 2.36E−01 | 3.42E−02 |
| | 224.G.S | | |
| 592 | 304.M.T | 2.19E−01 | 3.73E−02 |
| 788 | 891.S.Q | 2.18E−01 | 3.43E−02 |
| 583 | 169.L.K | 2.17E−01 | 3.66E−02 |
| 555 | 171.A.D | 2.14E−01 | 3.73E−02 |
| 515 | — | 2.09E−01 | 3.82E−02 |
| 569 | 9.K.G | 2.08E−01 | 3.12E−02 |
| 787 | 826.V.M | 2.07E−01 | 3.45E−02 |
| 561 | 5.—.G | 1.98E−01 | 3.41E−02 |
| 577 | 64.R.Q | 1.94E−01 | 3.48E−02 |
| 585 | 171.A.S | 1.94E−01 | 3.40E−02 |
| 572 | 35.R.P | 1.89E−01 | 3.95E−02 |
| 536 | 224.G.T | 1.88E−01 | 3.43E−02 |
| 656 | 887.T.D | 1.87E−01 | 3.34E−02 |
| 559 | 4.I.G | 1.87E−01 | 3.22E−02 |
| 777 | 169.L.Q | 1.84E−01 | 3.36E−02 |
| 584 | 171.A.Y | 1.83E−01 | 3.66E−02 |
| 779 | 372.G.I | 1.79E−01 | 3.15E−02 |
| 566 | 7.I.A | 1.77E−01 | 3.37E−02 |
| 638 | 481.E.D | 1.77E−01 | 3.37E−02 |
| 593 | 304.M.W | 1.76E−01 | 3.35E−02 |
| 568 | 8.N.S | 1.76E−01 | 3.34E−02 |
| 562 | 5.K.G | 1.74E−01 | 3.04E−02 |
| 564 | 6.—.G | 1.73E−01 | 3.32E−02 |
| 757 | 796.K.Q | 1.73E−01 | 2.94E−02 |
| 654 | 772.M.S | 1.70E−01 | 3.18E−02 |
| 760 | 797.T.V | 1.70E−01 | 3.12E−02 |
| 818 | 698.S.R | 1.67E−01 | 3.31E−02 |
| 646 | 653.—.T | 1.66E−01 | 3.06E−02 |
| 784 | 570.P.I | 1.65E−01 | 2.64E−02 |
| 762 | 793.—.P | 1.64E−01 | 2.99E−02 |
| 789 | 917.G.E | 1.61E−01 | 2.91E−02 |
| 649 | 655.—.S | 1.58E−01 | 3.13E−02 |
| 594 | 329.G.N | 1.56E−01 | 3.26E−02 |
| 604 | 342.—.A | 1.55E−01 | 2.87E−02 |
| 612 | 412.G.P | 1.55E−01 | 2.81E−02 |
| 644 | 593.Q.V | 1.52E−01 | 2.97E−02 |
| 736 | 794.P.A | 1.52E−01 | 2.85E−02 |
| 790 | 951.—.S | 1.51E−01 | 2.87E−02 |
| 780 | 389.—.V | 1.49E−01 | 3.02E−02 |
| 717 | 390.K.Q | 1.43E−01 | 2.84E−02 |
| 633 | 473.—.D | 1.41E−01 | 2.71E−02 |
| 590 | 289.K.S | 1.40E−01 | 2.92E−02 |
| 657 | 893.—.N | 1.39E−01 | 2.96E−02 |
| 643 | 593.Q.F | 1.38E−01 | 2.80E−02 |
| 544 | 232.D.G | 1.37E−01 | 3.19E−02 |
| 591 | 292.V.L | 1.37E−01 | 2.76E−02 |
| 534 | 224.G.H | 1.36E−01 | 2.24E−02 |
| 791 | 953.—.K | 1.34E−01 | 2.60E−02 |
| 781 | 390.K.E | 1.33E−01 | 2.71E−02 |
| 718 | 7.I.L | 1.32E−01 | 2.85E−02 |
| 812 | 329.G.K | 1.29E−01 | 2.97E−02 |
| 609 | 405.L.N | 1.26E−01 | 2.57E−02 |
| 758 | 791.E.N | 1.24E−01 | 2.33E−02 |
| 616 | 414.—.Y | 1.22E−01 | 2.44E−02 |
| 632 | 469.L.S | 1.22E−01 | 2.79E−02 |
| 614 | 414.—.R | 1.18E−01 | 2.32E−02 |
| 721 | 156.F.V | 1.14E−01 | 2.48E−02 |
| 611 | 408.E.Y | 1.11E−01 | 2.08E−02 |
| 622 | 420.D.G | 1.07E−01 | 2.41E−02 |
| 610 | 405.L.W | 1.06E−01 | 2.21E−02 |
| 619 | 417.K.D | 1.05E−01 | 2.59E−02 |
| 580 | 86.W.D | 1.00E−01 | 2.22E−02 |
| 602 | 339.—.Q | 9.82E−02 | 2.53E−02 |
| 537 | 224.G.A | 9.21E−02 | 2.34E−02 |
| 587 | 224.G.— | 8.18E−02 | 2.19E−02 |
| 538 | 224.G.V | 7.16E−02 | 2.16E−02 |
| 702 | 91.K.V | 6.31E−02 | 1.93E−02 |
| 824 | 611.K.Q | 4.07E−02 | 1.23E−02 |
| 631 | 469.L.K | 3.01E−02 | 1.30E−02 |
| 573 | 53.E.P | 2.65E−02 | 1.11E−02 |
| 528 | 224.G.Y | 8.98E−03 | 7.97E−03 |
| 535 | 224.G.S | 7.92E−03 | 7.31E−03 |
| Cas9 | n/a | 7.17E−03 | 8.11E−03 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

As shown in Table 66, CasX variants 607, 532, 676, 592, 788, 583, and 555 produced higher levels of on-target editing than did CasX variant 515. CasX variants 569, 787, 561, 577, 585, and 572 also produced relatively high levels of on-target editing, with at least 9000 of the activity of CasX variant 515 (i.e., greater than 1.88E-01 on-target editing).

Table 67 provides the level of off-target editing produced by various CasX variants with mutations relative to CasX variant 515, ranked from lowest to highest activity.

TABLE 67
Average off-targeting editing activity, ranked from lowest to highest

| Protein Designation | Mutation relative to | Average off-target TTC | SEM off-target TTC |
|---|---|---|---|
| (CasX variant no., | CasX 515* | PAM editing activity | PAM editing activity |
| or Cas9) | (Position.Reference.Alternative) | (fraction) | (fraction) |
| 528 | 224.G.Y | 1.73E−03 | 1.84E−03 |
| Cas9 | n/a | 2.18E−03 | 2.72E−03 |
| 535 | 224.G.S | 2.33E−03 | 2.63E−03 |
| 573 | 53.E.P | 5.01E−03 | 3.29E−03 |
| 824 | 611.K.Q | 5.86E−03 | 2.93E−03 |
| 631 | 469.L.K | 6.23E−03 | 3.45E−03 |
| 587 | 224.G.— | 1.15E−02 | 4.68E−03 |
| 538 | 224.G.V | 1.45E−02 | 7.45E−03 |
| 702 | 91.K.V | 1.52E−02 | 6.87E−03 |
| 812 | 329.G.K | 1.58E−02 | 7.16E−03 |
| 580 | 86.W.D | 1.78E−02 | 6.27E−03 |
| 619 | 417.K.D | 1.94E−02 | 6.80E−03 |
| 610 | 405.L.W | 2.08E−02 | 6.80E−03 |
| 758 | 791.E.N | 2.11E−02 | 7.51E−03 |
| 721 | 156.F.V | 2.12E−02 | 6.94E−03 |
| 591 | 292.V.L | 2.15E−02 | 7.81E−03 |
| 537 | 224.G.A | 2.18E−02 | 8.82E−03 |
| 590 | 289.K.S | 2.28E−02 | 8.23E−03 |
| 622 | 420.D.G | 2.30E−02 | 7.70E−03 |
| 632 | 469.L.S | 2.34E−02 | 7.92E−03 |
| 633 | 473.—.D | 2.36E−02 | 8.67E−03 |
| 614 | 414.—.R | 2.38E−02 | 7.57E−03 |
| 594 | 329.G.N | 2.41E−02 | 9.14E−03 |
| 643 | 593.Q.F | 2.46E−02 | 8.93E−03 |
| 609 | 405.L.N | 2.47E−02 | 8.24E−03 |
| 781 | 390.K.E | 2.48E−02 | 8.34E−03 |
| 616 | 414.—.Y | 2.48E−02 | 7.29E−03 |
| 602 | 339.—.Q | 2.55E−02 | 9.70E−03 |
| 791 | 953.—.K | 2.71E−02 | 7.83E−03 |
| 593 | 304.M.W | 2.87E−02 | 1.04E−02 |
| 644 | 593.Q.V | 2.88E−02 | 9.11E−03 |
| 657 | 893.—.N | 2.89E−02 | 1.02E−02 |
| 717 | 390.K.Q | 2.99E−02 | 1.00E−02 |
| 611 | 408.E.Y | 3.00E−02 | 8.86E−03 |
| 572 | 35.R.P | 3.06E−02 | 1.07E−02 |
| 780 | 389.—.V | 3.07E−02 | 9.83E−03 |
| 818 | 698.S.R | 3.15E−02 | 1.02E−02 |
| 638 | 481.E.D | 3.16E−02 | 1.01E−02 |
| 584 | 171.A.Y | 3.39E−02 | 1.07E−02 |
| 790 | 951.—.S | 3.47E−02 | 1.02E−02 |
| 718 | 7.I.L | 3.47E−02 | 1.06E−02 |
| 649 | 655.—.S | 3.54E−02 | 1.09E−02 |
| 562 | 5.K.G | 3.56E−02 | 1.09E−02 |
| 784 | 570.P.I | 3.60E−02 | 9.80E−03 |
| 736 | 794.P.A | 3.61E−02 | 1.03E−02 |
| 789 | 917.G.E | 3.63E−02 | 1.11E−02 |
| 544 | 232.D.G | 3.64E−02 | 1.15E−02 |
| 612 | 412.G.P | 3.64E−02 | 1.12E−02 |
| 604 | 342.—.A | 3.66E−02 | 1.11E−02 |
| 564 | 6.—.G | 3.67E−02 | 1.07E−02 |
| 568 | 8.N.S | 3.91E−02 | 1.15E−02 |
| 779 | 372.G.I | 3.98E−02 | 1.20E−02 |
| 760 | 797.T.V | 3.99E−02 | 1.09E−02 |
| 777 | 169.L.Q | 4.11E−02 | 1.16E−02 |
| 566 | 7.I.A | 4.20E−02 | 1.15E−02 |
| 569 | 9.K.G | 4.29E−02 | 1.03E−02 |
| 577 | 64.R.Q | 4.38E−02 | 1.28E−02 |
| 536 | 224.G.T | 4.44E−02 | 1.32E−02 |
| 656 | 887.T.D | 4.54E−02 | 1.22E−02 |
| 646 | 653.—.T | 4.56E−02 | 1.21E−02 |
| 757 | 796.K.Q | 4.60E−02 | 1.25E−02 |
| 559 | 4.I.G | 4.71E−02 | 1.19E−02 |
| 585 | 171.A.S | 4.73E−02 | 1.24E−02 |
| 515 | — | 4.85E−02 | 1.33E−02 |
| 762 | 793.—.P | 4.91E−02 | 1.25E−02 |
| 561 | 5.—.G | 4.92E−02 | 1.37E−02 |
| 788 | 891.S.Q | 5.50E−02 | 1.45E−02 |
| 555 | 171.A.D | 5.53E−02 | 1.52E−02 |
| 787 | 826.V.M | 5.68E−02 | 1.33E−02 |
| 654 | 772.M.S | 5.84E−02 | 1.61E−02 |
| 583 | 169.L.K | 5.95E−02 | 1.48E−02 |
| 592 | 304.M.T | 5.97E−02 | 1.48E−02 |
| 534 | 224.G.H | 6.37E−02 | 1.52E−02 |
| 676 | 27.—.R + 170.L.K + | 7.13E−02 | 1.68E−02 |
| | 224.G.S | | |
| 607 | 398.Y.T | 8.25E−02 | 1.82E−02 |
| 532 | 27.—.R | 8.70E−02 | 1.73E−02 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

As shown in Table 67, many of the tested CasX variants showed lower levels of off-target editing than did CasX variant 515. For example, consistent with previous results, CasX variant 812 produced relatively low levels of off-target editing. Further, some of the tested CasX proteins showed even lower levels of off-target editing than did CasX variant 812 (specifically, CasX variants 528, 535, 573, 824, 631, 587, 538, and 702).

Based on these results, a set of mutation conferring a high degree of editing activity and/or specificity was chosen for introducing in pairs into CasX variant 515. First, high activity mutations were defined as those that showed a level of on-target editing equal to at least 87.3% of the level of on-target editing by CasX variant 515. CasX variants 607, 532, 676, 592, 788, 583, 555, 569, 787, 561, 577, 585, 572, 536, 656, 559, 777, and 584 met this threshold, and were therefore selected as potential activity-enhancing mutations (see Table 68). Second, high specificity mutations were defined as those producing 80% or lower of the level of off-target editing produced by CasX variant 515, while maintaining at least 79.95% of the on-target editing activity of CasX variant 515. This 80% on-target editing activity requirement was implemented to avoid selecting mutations that were simply loss-of-function mutations and would therefore not be expected to be useful as gene editors. CasX variants 593, 572, 818, 638, 584, 562, and 784 met these criteria, and were therefore selected as potential specificity-enhancing mutations (see Table 68).

In total, 22 individual mutations were chosen as candidates for introducing in pairs into CasX variant 515 and testing for improved properties, as described in Example 38, below. The positions of the individual mutations relative to full-length CasX variant 515, as well as amino acid sequences of full-length CasX variants with the individual mutations, are provided in Table 68. Table 69, below, shows the amino acid sequences and coordinates of the domains of CasX variant 515, and Table 70 shows the positions of the 22 individual mutations within the domains of CasX variant 515, as well as the amino acid sequences of domains with each individual mutations.

TABLE 68
Summary of positions of single mutations
within the CasX 515 variant

| | | Mutation relative | Full-length CasX |
|---|---|---|---|
| CasX | | to CasX 515* | variant amino |
| variant | | (Position. Reference. | acid sequence |
| no. | Phenotype | Alternative) | (SEQ ID NO) |
| 532 | Activity | 27.-.R | 213 |
| 536 | Activity | 224.G.T | 217 |
| 555 | Activity | 171.A.D | 235 |
| 559 | Activity | 4.I.G | 239 |
| 561 | Activity | 5.-.G | 241 |
| 562 | Specificity | 5.K.G | 242 |
| 564 | Specificity | 6.-.G | 244 |
| 569 | Activity | 9.K.G | 249 |
| 572 | Activity (and | 35.R.P | 252 |
| | specificity) | | |
| 577 | Activity | 64.R.Q | 257 |
| 583 | Activity | 169.L.K | 263 |
| 584 | Activity (and | 171.A.Y | 264 |
| | specificity) | | |
| 585 | Activity | 171.A.S | 265 |
| 592 | Activity | 304.M.T | 272 |
| 593 | Specificity | 304.M.W | 273 |
| 607 | Activity | 398.Y.T | 287 |
| 638 | Specificity | 481.E.D | 318 |
| 656 | Activity | 887.T.D | 336 |
| 777 | Activity | 169.L.Q | 450 |
| 787 | Activity | 826.V.M | 460 |
| 788 | Activity | 891.S.Q | 461 |
| 818 | Specificity | 698.S.R | 490 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘-’ in the reference sequence (first position), and deletions with a ‘-’ in the alt sequence (second position). Multiple individual mutations are separated by “&”

TABLE 69
CasX variant 515 domain sequences and coordinates

| | SEQ | | |
|---|---|---|---|
| Domain | ID NO | Amino Acid Sequence | Coordinates |
| N-terminal | — | M | 1 |
| methionine | | | |
| OBD-I | 585 | QEIKRINKIRRRLVKDSNTKKAGKTGPMKTLLVRVMTPDLRERLEN | 2-57 |
| | | LRKKPENIPQ | |
| Helical I-I | 586 | PISNTSRANLNKLLTDYTEMKKAILHVYWEEFQKDPVGLMSRVA | 58-101 |
| NTSB | 587 | QPASKKIDQNKLKPEMDEKGNLTTAGFACSQCGQPLFVYKLEQVSE | 102-192 |
| | | KGKAYTNYFGRCNVAEHEKLILLAQLKPEKDSDEAVTYSLGKFGQ | |
| Helical I-II | 588 | RALDFYSIHVTKESTHPVKPLAQIAGNRYASGPVGKALSDACMGTI | 193-333 |
| | | ASFLSKYQDIIIEHQKVVKGNQKRLESLRELAGKENLEYPSVTLPP | |
| | | QPHTKEGVDAYNEVIARVRMWVNLNLWQKLKLSRDDAKPLLRLKGF | |
| | | PSF | |
| Helical II | 589 | PLVERQANEVDWWDMVCNVKKLINEKKEDGKVFWQNLAGYKRQEAL | 334-501 |
| | | RPYLSSEEDRKKGKKFARYQLGDLLLHLEKKHGEDWGKVYDEAWER | |
| | | IDKKVEGLSKHIKLEEERRSEDAQSKAALTDWLRAKASFVIEGLKE | |
| | | ADKDEFCRCELKLQKWYGDLRGKPFAIEAE | |
| OBD-II | 590 | NSILDISGFSKQYNCAFIWQKDGVKKLNLYLIINYFKGGKLRFKKI | 502-647 |
| | | KPEAFEANRFYTVINKKSGEIVPMEVNENFDDPNLIILPLAFGKRQ | |
| | | GREFIWNDLLSLETGSLKLANGRVIEKTLYNRRTRQDEPALFVALT | |
| | | FERREVLD | |
| RuvC-I | 591 | SSNIKPMNLIGVDRGENIPAVIALTDPEGCPLSRFKDSLGNPTHIL | 648-811 |
| | | RIGESYKEKQRTIQAKKEVEQRRAGGYSRKYASKAKNLADDMVRNT | |
| | | ARDLLYYAVTQDAMLIFENLSRGFGRQGKRTFMAERQYTRMEDWLT | |
| | | AKLAYEGLPSKTYLSKTLAQYTSKTC | |
| TSL | 592 | SNCGFTITSADYDRVLEKLKKTATGWMTTINGKELKVEGQITYYNR | 812-921 |
| | | YKRQNVVKDLSVELDRLSEESVNNDISSWTKGRSGEALSLLKKRES | |
| | | HRPVQEKFVCLNCGFETH | |
| RuvC-II | 593 | ADEQAALNIARSWLFLRSQEYKKYQTNKTTGNTDKRAFVETWQSFY | 922-979 |
| | | RKKLKEVWKPAV | |

TABLE 70
Summary of positions of single mutations within CasX 515 variant domains

| | | Position of | | |
|---|---|---|---|---|
| | | mutation within | | Amino acid |
| | | CasX 515 domain* | | sequence of |
| CasX | | (Position. | | mutated |
| variant | Mutated | Reference. | Amino acid sequence of | domain |
| no. | domain | Alternative) | mutated domain† | (SEQ ID NO) |
| 532 | OBD-I | 26.-.R | QEIKRINKIRRRLVKDSNTKKAGKTRGPMK | 9543 |
| | | | TLLVRVMTPDLRERLENLRKKPENIPQ | |
| 536 | Helical I-II | 32.G.T | RALDFYSIHVTKESTHPVKPLAQIAGNRYA | 9544 |
| | | | STPVGKALSDACMGTIASFLSKYQDIIIEH | |
| | | | QKVVKGNQKRLESLRELAGKENLEYPSVTL | |
| | | | PPQPHTKEGVDAYNEVIARVRMWVNLNLWQ | |
| | | | KLKLSRDDAKPLLRLKGFPSF | |
| 555 | NTSB | 70.A.D | QPASKKIDQNKLKPEMDEKGNLTTAGFACS | 9545 |
| | | | QCGQPLFVYKLEQVSEKGKAYTNYFGRCNV | |
| | | | AEHEKLILLDQLKPEKDSDEAVTYSLGKFG | |
| | | | Q | |
| 559 | OBD-I | 3.I.G | QEGKRINKIRRRLVKDSNTKKAGKTGPMKT | 9546 |
| | | | LLVRVMTPDLRERLENLRKKPENIPQ | |
| 561 | OBD-I | 4.-.G | QEIGKRINKIRRRLVKDSNTKKAGKTGPMK | 9547 |
| | | | TLLVRVMTPDLRERLENLRKKPENIPQ | |
| 562 | OBD-I | 4.K.G | QEIGRINKIRRRLVKDSNTKKAGKTGPMKT | 9548 |
| | | | LLVRVMTPDLRERLENLRKKPENIPQ | |
| 564 | OBD-I | 5.-.G | QEIKGRINKIRRRLVKDSNTKKAGKTGPMK | 9549 |
| | | | TLLVRVMTPDLRERLENLRKKPENIPQ | |
| 569 | OBD-I | 8.K.G | QEIKRINGIRRRLVKDSNTKKAGKTGPMKT | 9550 |
| | | | LLVRVMTPDLRERLENLRKKPENIPQ | |
| 572 | OBD-I | 34.R.P | QEIKRINKIRRRLVKDSNTKKAGKTGPMKT | 9551 |
| | | | LLVPVMTPDLRERLENLRKKPENIPQ | |
| 577 | Helical I-I | 7.R.Q | PISNTSQANLNKLLTDYTEMKKAILHVYWE | 9552 |
| | | | EFQKDPVGLMSRVA | |
| 583 | NTSB | 68.L.K | QPASKKIDQNKLKPEMDEKGNLTTAGFACS | 9553 |
| | | | QCGQPLFVYKLEQVSEKGKAYTNYFGRCNV | |
| | | | AEHEKLIKLAQLKPEKDSDEAVTYSLGKFG | |
| | | | Q | |
| 584 | NTSB | 70.A.Y | QPASKKIDQNKLKPEMDEKGNLTTAGFACS | 9554 |
| | | | QCGQPLFVYKLEQVSEKGKAYTNYFGRCNV | |
| | | | AEHEKLILLYQLKPEKDSDEAVTYSLGKFG | |
| | | | Q | |
| 585 | NTSB | 70.A.S | QPASKKIDQNKLKPEMDEKGNLTTAGFACS | 9555 |
| | | | QCGQPLFVYKLEQVSEKGKAYTNYFGRCNV | |
| | | | AEHEKLILLSQLKPEKDSDEAVTYSLGKFG | |
| | | | Q | |
| 592 | Helical I-II | 112.M.T | RALDFYSIHVTKESTHPVKPLAQIAGNRYA | 9556 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRTWVNLNLWQ | |
| | | | KLKLSRDDAKPLLRLKGFPSF | |
| 593 | Helical I-II | 112.M.W | RALDFYSIHVTKESTHPVKPLAQIAGNRYA | 9557 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRWWVNLNLWQ | |
| | | | KLKLSRDDAKPLLRLKGFPSF | |
| 607 | Helical II | 65.Y.T | PLVERQANEVDWWDMVCNVKKLINEKKEDG | 9558 |
| | | | KVFWQNLAGYKRQEALRPYLSSEEDRKKGK | |
| | | | KFARTQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCELK | |
| | | | LQKWYGDLRGKPFAIEAE | |
| 638 | Helical II | 148.E.D | PLVERQANEVDWWDMVCNVKKLINEKKEDG | 9559 |
| | | | KVFWQNLAGYKRQEALRPYLSSEEDRKKGK | |
| | | | KFARYQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCDLK | |
| | | | LQKWYGDLRGKPFAIEAE | |
| 656 | TSL | 76.T.D | SNCGFTITSADYDRVLEKLKKTATGWMTTI | 9560 |
| | | | NGKELKVEGQITYYNRYKRQNVVKDLSVEL | |
| | | | DRLSEESVNNDISSWDKGRSGEALSLLKKR | |
| | | | FSHRPVQEKFVCLNCGFETH | |
| 777 | NTSB | 68.L.Q | QPASKKIDQNKLKPEMDEKGNLTTAGFACS | 9561 |
| | | | QCGQPLFVYKLEQVSEKGKAYTNYFGRCNV | |
| | | | AEHEKLIQLAQLKPEKDSDEAVTYSLGKFG | |
| | | | Q | |
| 787 | TSL | 15.V.M | SNCGFTITSADYDRMLEKLKKTATGWMTTI | 9562 |
| | | | NGKELKVEGQITYYNRYKRQNVVKDLSVEL | |
| | | | DRLSEESVNNDISSWTKGRSGEALSLLKKR | |
| | | | FSHRPVQEKFVCLNCGFETH | |
| 788 | TSL | 80.S.Q | SNCGFTITSADYDRVLEKLKKTATGWMTTI | 9563 |
| | | | NGKELKVEGQITYYNRYKRQNVVKDLSVEL | |
| | | | DRLSEESVNNDISSWTKGRQGEALSLLKKR | |
| | | | FSHRPVQEKFVCLNCGFETH | |
| 818 | RuvC-I | 51.S.R | SSNIKPMNLIGVDRGENIPAVIALTDPEGC | 9564 |
| | | | PLSRFKDSLGNPTHILRIGERYKEKQRTIQ | |
| | | | AKKEVEQRRAGGYSRKYASKAKNLADDMVR | |
| | | | NTARDLLYYAVTQDAMLIFENLSRGFGRQG | |
| | | | KRTFMAERQYTRMEDWLTAKLAYEGLPSKT | |
| | | | YLSKTLAQYTSKTC | |

*Positions of mutations within domains are shown relative to the CasX 515 domain sequences provided in Table 69, above. Each mutation is indicated by its position, reference sequence, and alt sequence, separated by ‘.’ Insertions are indicated with a ‘—’ in the reference sequence (first position), and deletions with a ‘—’ in the alt sequence (second position).
†Mutated residues are bolded and underlined.

### Example 38: CasX Variants with Two or Three Mutations Relative to CasX Variant 515

CasX variants were generated with two or three mutations relative to CasX variant 515, and assessed for their on and off-target gene editing activity.

### Materials and Methods

Pairs of mutations listed in Tables 68 and 70, above, were introduced into the CasX variant 515 amino acid sequence to generate 161 amino acid sequences of CasX variants. The pairs of mutations and full-length amino acid sequences of the 161 CasX variants tested are listed in Table 71, and Table 72 provides the amino acid sequences of each of the domains of the 161 CasX variants.

TABLE 71
Pairs of mutations and amino acid sequences of CasX variants

| Mutations relative | Phenotypes of | Full-length |
|---|---|---|
| to CasX 515* (Position. | single mutations | CasX amino acid |
| Reference. Alternative) | (see Example 37) | SEQ ID NO |
| 4.I.G & 64.R.Q | Activity + activity | 9382 |
| 4.I.G & 169.L.K | Activity + activity | 9383 |
| 4.I.G & 169.L.Q | Activity + activity | 9384 |
| 4.I.G & 171.A.D | Activity + activity | 9385 |
| 4.I.G & 171.A.Y | Activity + activity | 9386 |
| 4.I.G & 171.A.S | Activity + activity | 9387 |
| 4.I.G & 224.G.T | Activity + activity | 9388 |
| 4.I.G & 304.M.T | Activity + activity | 9389 |
| 4.I.G & 398.Y.T | Activity + activity | 9390 |
| 4.I.G & 826.V.M | Activity + activity | 9391 |
| 4.I.G & 887.T.D | Activity + activity | 9392 |
| 4.I.G & 891.S.Q | Activity + activity | 9393 |
| 5.-.G & 64.R.Q | Activity + activity | 9394 |
| 5.-.G & 169.L.K | Activity + activity | 9395 |
| 5.-.G & 169.L.Q | Activity + activity | 9396 |
| 5.-.G & 171.A.D | Activity + activity | 9397 |
| 5.-.G & 171.A.Y | Activity + activity | 9398 |
| 5.-.G & 171.A.S | Activity + activity | 9399 |
| 5.-.G & 224.G.T | Activity + activity | 9400 |
| 5.-.G & 304.M.T | Activity + activity | 9401 |
| 5.-.G & 398.Y.T | Activity + activity | 9402 |
| 5.-.G & 826.V.M | Activity + activity | 9403 |
| 5.-.G & 887.T.D | Activity + activity | 9404 |
| 5.-.G & 891.S.Q | Activity + activity | 9405 |
| 9.K.G & 64.R.Q | Activity + activity | 9406 |
| 9.K.G & 169.L.K | Activity + activity | 9407 |
| 9.K.G & 169.L.Q | Activity + activity | 9408 |
| 9.K.G & 171.A.D | Activity + activity | 9409 |
| 9.K.G & 171.A.Y | Activity + activity | 9410 |
| 9.K.G & 171.A.S | Activity + activity | 9411 |
| 9.K.G & 224.G.T | Activity + activity | 9412 |
| 9.K.G & 304.M.T | Activity + activity | 9413 |
| 9.K.G & 398.Y.T | Activity + activity | 9414 |
| 9.K.G & 826.V.M | Activity + activity | 9415 |
| 9.K.G & 887.T.D | Activity + activity | 9416 |
| 9.K.G & 891.S.Q | Activity + activity | 9417 |
| 27.-.R & 64.R.Q | Activity + activity | 9418 |
| 27.-.R & 169.L.K | Activity + activity | 9419 |
| 27.-.R & 169.L.Q | Activity + activity | 9420 |
| 27.-.R & 171.A.D | Activity + activity | 9421 |
| 27.-.R & 171.A.Y | Activity + activity | 9422 |
| 27.-.R & 171.A.S | Activity + activity | 9423 |
| 27.-.R & 224.G.T | Activity + activity | 9424 |
| 27.-.R & 304.M.T | Activity + activity | 9425 |
| 27.-.R & 398.Y.T | Activity + activity | 9426 |
| 27.-.R & 826.V.M | Activity + activity | 9427 |
| 27.-.R & 887.T.D | Activity + activity | 9428 |
| 27.-.R & 891.S.Q | Activity + activity | 9429 |
| 35.R.P & 64.R.Q | Activity + activity | 9430 |
| 35.R.P & 169.L.K | Activity + activity | 9431 |
| 35.R.P & 169.L.Q | Activity + activity | 9432 |
| 35.R.P & 171.A.D | Activity + activity | 9433 |
| 35.R.P & 171.A.Y | Activity + activity | 9434 |
| 35.R.P & 171.A.S | Activity + activity | 9435 |
| 35.R.P & 224.G.T | Activity + activity | 9436 |
| 35.R.P & 304.M.T | Activity + activity | 9437 |
| 35.R.P & 398.Y.T | Activity + activity | 9438 |
| 35.R.P & 826.V.M | Activity + activity | 9439 |
| 35.R.P & 887.T.D | Activity + activity | 9440 |
| 35.R.P & 891.S.Q | Activity + activity | 9441 |
| 887.T.D & 891.S.Q | Activity + activity | 9442 |
| 64.R.Q & 169.L.K | Activity + activity | 9443 |
| 64.R.Q & 169.L.Q | Activity + activity | 9444 |
| 64.R.Q & 171.A.D | Activity + activity | 9445 |
| 64.R.Q & 171.A.Y | Activity + activity | 9446 |
| 64.R.Q & 171.A.S | Activity + activity | 9447 |
| 64.R.Q & 224.G.T | Activity + activity | 9448 |
| 64.R.Q & 304.M.T | Activity + activity | 9449 |
| 64.R.Q & 398.Y.T | Activity + activity | 9450 |
| 64.R.Q & 826.V.M | Activity + activity | 9451 |
| 64.R.Q & 887.T.D | Activity + activity | 9452 |
| 64.R.Q & 891.S.Q | Activity + activity | 9453 |
| 169.L.K & 171.A.D | Activity + activity | 9454 |
| 169.L.K & 171.A.Y | Activity + activity | 9455 |
| 169.L.K & 171.A.S | Activity + activity | 9456 |
| 169.L.K & 224.G.T | Activity + activity | 9457 |
| 169.L.K & 304.M.T | Activity + activity | 9458 |
| 169.L.K & 398.Y.T | Activity + activity | 9459 |
| 169.L.K & 826.V.M | Activity + activity | 9460 |
| 169.L.K & 887.T.D | Activity + activity | 9461 |
| 169.L.K & 891.S.Q | Activity + activity | 9462 |
| 169.L.Q & 171.A.D | Activity + activity | 9463 |
| 169.L.Q & 171.A.Y | Activity + activity | 9464 |
| 169.L.Q & 171.A.S | Activity + activity | 9465 |
| 169.L.Q & 224.G.T | Activity + activity | 9466 |
| 169.L.Q & 304.M.T | Activity + activity | 9467 |
| 169.L.Q & 398.Y.T | Activity + activity | 9468 |
| 169.L.Q & 826.V.M | Activity + activity | 9469 |
| 169.L.Q & 887.T.D | Activity + activity | 9470 |
| 169.L.Q & 891.S.Q | Activity + activity | 9471 |
| 171.A.D & 224.G.T | Activity + activity | 9472 |
| 171.A.D & 304.M.T | Activity + activity | 9473 |
| 171.A.D & 398.Y.T | Activity + activity | 9474 |
| 171.A.D & 826.V.M | Activity + activity | 9475 |
| 171.A.D & 887.T.D | Activity + activity | 9476 |
| 171.A.D & 891.S.Q | Activity + activity | 9477 |
| 171.A.Y & 224.G.T | Activity + activity | 9478 |
| 171.A.Y & 304.M.T | Activity + activity | 9479 |
| 171.A.Y & 398.Y.T | Activity + activity | 9480 |
| 171.A.Y & 826.V.M | Activity + activity | 9481 |
| 171.A.Y & 887.T.D | Activity + activity | 9482 |
| 171.A.Y & 891.S.Q | Activity + activity | 9483 |
| 171.A.S & 224.G.T | Activity + activity | 9484 |
| 171.A.S & 304.M.T | Activity + activity | 9485 |
| 171.A.S & 398.Y.T | Activity + activity | 9486 |
| 171.A.S & 826.V.M | Activity + activity | 9487 |
| 171.A.S & 887.T.D | Activity + activity | 9488 |
| 171.A.S & 891.S.Q | Activity + activity | 9489 |
| 4.I.G & 35.R.P | Activity + activity | 9490 |
| 224.G.T & 304.M.T | Activity + activity | 9491 |
| 224.G.T & 398.Y.T | Activity + activity | 9492 |
| 224.G.T & 826.V.M | Activity + activity | 9493 |
| 224.G.T & 887.T.D | Activity + activity | 9494 |
| 224.G.T & 891.S.Q | Activity + activity | 9495 |
| 5.-.G & 35.R.P | Activity + activity | 9496 |
| 4.I.G & 27.-.R | Activity + activity | 9497 |
| 304.M.T & 398.Y.T | Activity + activity | 9498 |
| 304.M.T & 826.V.M | Activity + activity | 9499 |
| 304.M.T & 887.T.D | Activity + activity | 9500 |
| 304.M.T & 891.S.Q | Activity + activity | 9501 |
| 9.K.G & 35.R.P | Activity + activity | 9502 |
| 5.-.G & 27.-.R | Activity + activity | 9503 |
| 4.I.G & 9.K.G | Activity + activity | 9504 |
| 398.Y.T & 826.V.M | Activity + activity | 9505 |
| 398.Y.T & 887.T.D | Activity + activity | 9506 |
| 398.Y.T & 891.S.Q | Activity + activity | 9507 |
| 27.-.R & 35.R.P | Activity + activity | 9508 |
| 9.K.G & 27.-.R | Activity + activity | 9509 |
| 5.-.G & 9.K.G | Activity + activity | 9510 |
| 4.I.G & 5.-.G | Activity + activity | 9511 |
| 826.V.M & 887.T.D | Activity + activity | 9512 |
| 826.V.M & 891.S.Q | Activity + activity | 9513 |
| 5.K.G & 27.-.R | Activity + specificity | 9514 |
| 5.K.G & 169.L.K | Activity + specificity | 9515 |
| 5.K.G & 171.A.D | Activity + specificity | 9516 |
| 5.K.G & 304.M.T | Activity + specificity | 9517 |
| 5.K.G & 398.Y.T | Activity + specificity | 9518 |
| 5.K.G & 891.S.Q | Activity + specificity | 9519 |
| 6.-.G & 27.-.R | Activity + specificity | 9520 |
| 6.-.G & 169.L.K | Activity + specificity | 9521 |
| 6.-.G & 171.A.D | Activity + specificity | 9522 |
| 6.-.G & 304.M.T | Activity + specificity | 9523 |
| 6.-.G & 398.Y.T | Activity + specificity | 9524 |
| 6.-.G & 891.S.Q | Activity + specificity | 9525 |
| 304.M.W & 27.-.R | Activity + specificity | 9526 |
| 304.M.W & 169.L.K | Activity + specificity | 9527 |
| 304.M.W & 171.A.D | Activity + specificity | 9528 |
| 304.M.W & 398.Y.T | Activity + specificity | 9529 |
| 304.M.W & 891.S.Q | Activity + specificity | 9530 |
| 481.E.D & 27.-.R | Activity + specificity | 9531 |
| 481.E.D & 169.L.K | Activity + specificity | 9532 |
| 481.E.D & 171.A.D | Activity + specificity | 9533 |
| 481.E.D & 304.M.T | Activity + specificity | 9534 |
| 481.E.D & 398.Y.T | Activity + specificity | 9535 |
| 481.E.D & 891.S.Q | Activity + specificity | 9536 |
| 698.S.R & 27.-.R | Activity + specificity | 9537 |
| 698.S.R & 169.L.K | Activity + specificity | 9538 |
| 698.S.R & 171.A.D | Activity + specificity | 9539 |
| 698.S.R & 304.M.T | Activity + specificity | 9540 |
| 698.S.R & 398.Y.T | Activity + specificity | 9541 |
| 698.S.R & 891.S.Q | Activity + specificity | 9542 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘-’ in the reference sequence (first position), and deletions with a ‘-’ in the alt sequence (second position). Multiple individual mutations are separated by “&”.

TABLE 72
Amino acid sequences of domains of CasX variants, N- to C-terminus

| Mutations relative | | | | | | | | | |
|---|---|---|---|---|---|---|---|---|---|
| to CasX 515* | Amino acid sequences of domains of CasX variants (SEQ ID NOs) | | | | | | | | |
| (Position. Reference. | OBD- | Helical | | Helical | Helical | OBD- | RuvC- | | RuvC- |
| Alternative) | I | I-I | NTSB | I-II | II | II | I | TSL | II |
| None (non-mutated | 585 | 586 | 587 | 588 | 589 | 590 | 591 | 592 | 593 |
| CasX 515) | | | | | | | | | |
| 4.I.G & 64.R.Q | 9546 | 9552 | 587 | 588 | 589 | 590 | 591 | 592 | 593 |
| 4.I.G & 169.L.K | 9546 | 586 | 9553 | 588 | 589 | 590 | 591 | 592 | 593 |
| 4.I.G & 169.L.Q | 9546 | 586 | 9561 | 588 | 589 | 590 | 591 | 592 | 593 |
| 4.I.G & 171.A.D | 9546 | 586 | 9545 | 588 | 589 | 590 | 591 | 592 | 593 |
| 4.I.G & 171.A.Y | 9546 | 586 | 9554 | 588 | 589 | 590 | 591 | 592 | 593 |
| 4.I.G & 171.A.S | 9546 | 586 | 9555 | 588 | 589 | 590 | 591 | 592 | 593 |
| 4.I.G & 224.G.T | 9546 | 586 | 587 | 9544 | 589 | 590 | 591 | 592 | 593 |
| 4.I.G & 304.M.T | 9546 | 586 | 587 | 9556 | 589 | 590 | 591 | 592 | 593 |
| 4.I.G & 398.Y.T | 9546 | 586 | 587 | 588 | 9558 | 590 | 591 | 592 | 593 |
| 4.I.G & 826.V.M | 9546 | 586 | 587 | 588 | 589 | 590 | 591 | 9562 | 593 |
| 4.I.G & 887.T.D | 9546 | 586 | 587 | 588 | 589 | 590 | 591 | 9560 | 593 |
| 4.I.G & 891.S.Q | 9546 | 586 | 587 | 588 | 589 | 590 | 591 | 9563 | 593 |
| 5.—.G & 64.R.Q | 9547 | 9552 | 587 | 588 | 589 | 590 | 591 | 592 | 593 |
| 5.—.G & 169.L.K | 9547 | 586 | 9553 | 588 | 589 | 590 | 591 | 592 | 593 |
| 5.—.G & 169.L.Q | 9547 | 586 | 9561 | 588 | 589 | 590 | 591 | 592 | 593 |
| 5.—.G & 171.A.D | 9547 | 586 | 9545 | 588 | 589 | 590 | 591 | 592 | 593 |
| 5.—.G & 171.A.Y | 9547 | 586 | 9554 | 588 | 589 | 590 | 591 | 592 | 593 |
| 5.—.G & 171.A.S | 9547 | 586 | 9555 | 588 | 589 | 590 | 591 | 592 | 593 |
| 5.—.G & 224.G.T | 9547 | 586 | 587 | 9544 | 589 | 590 | 591 | 592 | 593 |
| 5.—.G & 304.M.T | 9547 | 586 | 587 | 9556 | 589 | 590 | 591 | 592 | 593 |
| 5.—.G & 398.Y.T | 9547 | 586 | 587 | 588 | 9558 | 590 | 59 | 592 | 593 |
| 5.—.G & 826.V.M | 9547 | 586 | 587 | 588 | 589 | 590 | 591 | 9562 | 593 |
| 5.—.G & 887.T.D | 9547 | 586 | 587 | 588 | 589 | 590 | 591 | 9560 | 593 |
| 5.—.G & 891.S.Q | 9547 | 586 | 587 | 588 | 589 | 590 | 591 | 9563 | 593 |
| 9.K.G & 64.R.Q | 9550 | 9552 | 587 | 588 | 589 | 590 | 591 | 592 | 593 |
| 9.K.G & 169.L.K | 9550 | 586 | 9553 | 588 | 589 | 590 | 591 | 592 | 593 |
| 9.K.G & 169.L.Q | 9550 | 586 | 9561 | 588 | 589 | 590 | 591 | 592 | 593 |
| 9.K.G & 171.A.D | 9550 | 586 | 9545 | 588 | 589 | 590 | 591 | 592 | 593 |
| 9.K.G & 171.A.Y | 9550 | 586 | 9554 | 588 | 589 | 590 | 591 | 592 | 593 |
| 9.K.G & 171.A.S | 9550 | 586 | 9555 | 588 | 589 | 590 | 591 | 592 | 593 |
| 9.K.G & 224.G.T | 9550 | 586 | 587 | 9544 | 589 | 590 | 591 | 592 | 593 |
| 9.K.G & 304.M.T | 9550 | 586 | 587 | 9556 | 589 | 590 | 59 | 592 | 593 |
| 9.K.G & 398.Y.T | 9550 | 586 | 587 | 588 | 9558 | 590 | 591 | 592 | 593 |
| 9.K.G & 826.V.M | 9550 | 586 | 587 | 588 | 589 | 590 | 59 | 9562 | 593 |
| 9.K.G & 887.T.D | 9550 | 586 | 587 | 588 | 589 | 590 | 591 | 9560 | 593 |
| 9.K.G & 891.S.Q | 9550 | 586 | 587 | 588 | 589 | 590 | 591 | 9563 | 593 |
| 27.—.R & 64.R.Q | 9543 | 9552 | 587 | 588 | 589 | 590 | 591 | 592 | 593 |
| 27.—.R & 169.L.K | 9543 | 586 | 9553 | 588 | 589 | 590 | 591 | 592 | 593 |
| 27.—.R & 169.L.Q | 9543 | 586 | 9561 | 588 | 589 | 590 | 591 | 592 | 593 |
| 27.—.R & 171.A.D | 9543 | 586 | 9545 | 588 | 589 | 590 | 591 | 592 | 593 |
| 27.—.R & 171.A.Y | 9543 | 586 | 9554 | 588 | 589 | 590 | 591 | 592 | 593 |
| 27.—.R & 171.A.S | 9543 | 586 | 9555 | 588 | 589 | 590 | 591 | 592 | 593 |
| 27.—.R & 224.G.T | 9543 | 586 | 587 | 9544 | 589 | 590 | 591 | 592 | 593 |
| 27.—.R & 304.M.T | 9543 | 586 | 587 | 9556 | 589 | 590 | 591 | 592 | 593 |
| 27.—.R & 398.Y.T | 9543 | 586 | 587 | 588 | 9558 | 590 | 591 | 592 | 593 |
| 27.—.R & 826.V.M | 9543 | 586 | 587 | 588 | 589 | 590 | 591 | 9562 | 593 |
| 27.—.R & 887.T.D | 9543 | 586 | 587 | 588 | 589 | 590 | 591 | 9560 | 593 |
| 27.—.R & 891.S.Q | 9543 | 586 | 587 | 588 | 589 | 590 | 591 | 9563 | 593 |
| 35.R.P & 64.R.Q | 9551 | 9552 | 587 | 588 | 589 | 590 | 591 | 592 | 593 |
| 35.R.P & 169.L.K | 9551 | 586 | 9553 | 588 | 589 | 590 | 591 | 592 | 593 |
| 35.R.P & 169.L.Q | 9551 | 586 | 9561 | 588 | 589 | 590 | 591 | 592 | 593 |
| 35.R.P & 171.A.D | 9551 | 586 | 9545 | 588 | 589 | 590 | 591 | 592 | 593 |
| 35.R.P & 171.A.Y | 9551 | 586 | 9554 | 588 | 589 | 590 | 591 | 592 | 593 |
| 35.R.P & 171.A.S | 9551 | 586 | 9555 | 588 | 589 | 590 | 591 | 592 | 593 |
| 35.R.P & 224.G.T | 9551 | 586 | 587 | 9544 | 589 | 590 | 591 | 592 | 593 |
| 35.R.P & 304.M.T | 9551 | 586 | 587 | 9556 | 589 | 590 | 591 | 592 | 593 |
| 35.R.P & 398.Y.T | 9551 | 586 | 587 | 588 | 9558 | 590 | 591 | 592 | 593 |
| 35.R.P & 826.V.M | 9551 | 586 | 587 | 588 | 589 | 590 | 591 | 9562 | 593 |
| 35.R.P & 887.T.D | 9551 | 586 | 587 | 588 | 589 | 590 | 591 | 9560 | 593 |
| 35.R.P & 891.S.Q | 9551 | 586 | 587 | 588 | 589 | 590 | 591 | 9563 | 593 |
| 887.T.D & 891.S.Q | 585 | 586 | 587 | 588 | 589 | 590 | 591 | 9585 | 593 |
| 64.R.Q & 169.L.K | 585 | 9552 | 9553 | 588 | 589 | 590 | 591 | 592 | 593 |
| 64.R.Q & 169.L.Q | 585 | 9552 | 9561 | 588 | 589 | 590 | 591 | 592 | 593 |
| 64.R.Q & 171.A.D | 585 | 9552 | 9545 | 588 | 589 | 590 | 591 | 592 | 593 |
| 64.R.Q & 171.A.Y | 585 | 9552 | 9554 | 588 | 589 | 590 | 591 | 592 | 593 |
| 64.R.Q & 171.A.S | 585 | 9552 | 9555 | 588 | 589 | 590 | 591 | 592 | 593 |
| 64.R.Q & 224.G.T | 585 | 9552 | 587 | 9544 | 589 | 590 | 591 | 592 | 593 |
| 64.R.Q & 304.M.T | 585 | 9552 | 587 | 9556 | 589 | 590 | 591 | 592 | 593 |
| 64.R.Q & 398.Y.T | 585 | 9552 | 587 | 588 | 9558 | 590 | 591 | 592 | 593 |
| 64.R.Q & 826.V.M | 585 | 9552 | 587 | 588 | 589 | 590 | 591 | 9562 | 593 |
| 64.R.Q & 887.T.D | 585 | 9552 | 587 | 588 | 589 | 590 | 591 | 9560 | 593 |
| 64.R.Q & 891.S.Q | 585 | 9552 | 587 | 588 | 589 | 590 | 591 | 9563 | 593 |
| 169.L.K & 171.A.D | 585 | 586 | 9577 | 588 | 589 | 590 | 591 | 592 | 593 |
| 169.L.K & 171.A.Y | 585 | 586 | 9578 | 588 | 589 | 590 | 591 | 592 | 593 |
| 169.L.K & 171.A.S | 585 | 586 | 9579 | 588 | 589 | 590 | 591 | 592 | 593 |
| 169.L.K & 224.G.T | 585 | 586 | 9553 | 9544 | 589 | 590 | 591 | 592 | 593 |
| 169.L.K & 304.M.T | 585 | 586 | 9553 | 9556 | 589 | 590 | 591 | 592 | 593 |
| 169.L.K & 398.Y.T | 585 | 586 | 9553 | 588 | 9558 | 590 | 591 | 592 | 593 |
| 169.L.K & 826.V.M | 585 | 586 | 9553 | 588 | 589 | 590 | 591 | 9562 | 593 |
| 169.L.K & 887.T.D | 585 | 586 | 9553 | 588 | 589 | 590 | 591 | 9560 | 593 |
| 169.L.K & 891.S.Q | 585 | 586 | 9553 | 588 | 589 | 590 | 591 | 9563 | 593 |
| 169.L.Q & 171.A.D | 585 | 586 | 9580 | 588 | 589 | 590 | 591 | 592 | 593 |
| 169.L.Q & 171.A.Y | 585 | 586 | 9581 | 588 | 589 | 590 | 591 | 592 | 593 |
| 169.L.Q & 171.A.S | 585 | 586 | 9582 | 588 | 589 | 590 | 591 | 592 | 593 |
| 169.L.Q & 224.G.T | 585 | 586 | 9561 | 9544 | 589 | 590 | 591 | 592 | 593 |
| 169.L.Q & 304.M.T | 585 | 586 | 9561 | 9556 | 589 | 590 | 591 | 592 | 593 |
| 169.L.Q & 398.Y.T | 585 | 586 | 9561 | 588 | 9558 | 590 | 591 | 592 | 593 |
| 169.L.Q & 826.V.M | 585 | 586 | 9561 | 588 | 589 | 590 | 591 | 9562 | 593 |
| 169.L.Q & 887.T.D | 585 | 586 | 9561 | 588 | 589 | 590 | 591 | 9560 | 593 |
| 169.L.Q & 891.S.Q | 585 | 586 | 9561 | 588 | 589 | 590 | 591 | 9563 | 593 |
| 171.A.D & 224.G.T | 585 | 586 | 9545 | 9544 | 589 | 590 | 591 | 592 | 593 |
| 171.A.D & 304.M.T | 585 | 586 | 9545 | 9556 | 589 | 590 | 591 | 592 | 593 |
| 171.A.D & 398.Y.T | 585 | 586 | 9545 | 588 | 9558 | 590 | 591 | 592 | 593 |
| 171.A.D & 826.V.M | 585 | 586 | 9545 | 588 | 589 | 590 | 591 | 9562 | 593 |
| 171.A.D & 887.T.D | 585 | 586 | 9545 | 588 | 589 | 590 | 591 | 9560 | 593 |
| 171.A.D & 891.S.Q | 585 | 586 | 9545 | 588 | 589 | 590 | 591 | 9563 | 593 |
| 171.A.Y & 224.G.T | 585 | 586 | 9554 | 9544 | 589 | 590 | 591 | 592 | 593 |
| 171.A.Y & 304.M.T | 585 | 586 | 9554 | 9556 | 589 | 590 | 591 | 592 | 593 |
| 171.A.Y & 398.Y.T | 585 | 586 | 9554 | 588 | 9558 | 590 | 591 | 592 | 593 |
| 171.A.Y & 826.V.M | 585 | 586 | 9554 | 588 | 589 | 590 | 591 | 9562 | 593 |
| 171.A.Y & 887.T.D | 585 | 586 | 9554 | 588 | 589 | 590 | 591 | 9560 | 593 |
| 171.A.Y & 891.S.Q | 585 | 586 | 9554 | 588 | 589 | 590 | 591 | 9563 | 593 |
| 171.A.S & 224.G.T | 585 | 586 | 9555 | 9544 | 589 | 590 | 591 | 592 | 593 |
| 171.A.S & 304.M.T | 585 | 586 | 9555 | 9556 | 589 | 590 | 591 | 592 | 593 |
| 171.A.S & 398.Y.T | 585 | 586 | 9555 | 588 | 9558 | 590 | 591 | 592 | 593 |
| 171.A.S & 826.V.M | 585 | 586 | 9555 | 588 | 589 | 590 | 591 | 9562 | 593 |
| 171.A.S & 887.T.D | 585 | 586 | 9555 | 588 | 589 | 590 | 591 | 9560 | 593 |
| 171.A.S & 891.S.Q | 585 | 586 | 9555 | 588 | 589 | 590 | 591 | 9563 | 593 |
| 4.I.G & 35.R.P | 9565 | 586 | 587 | 588 | 589 | 590 | 591 | 592 | 593 |
| 224.G.T & 304.M.T | 585 | 586 | 587 | 9583 | 589 | 590 | 591 | 592 | 593 |
| 224.G.T & 398.Y.T | 585 | 586 | 587 | 9544 | 9558 | 590 | 591 | 592 | 593 |
| 224.G.T & 826.V.M | 585 | 586 | 587 | 9544 | 589 | 590 | 591 | 9562 | 593 |
| 224.G.T & 887.T.D | 585 | 586 | 587 | 9544 | 589 | 590 | 591 | 9560 | 593 |
| 224.G.T & 891.S.Q | 585 | 586 | 587 | 9544 | 589 | 590 | 591 | 9563 | 593 |
| 5.—.G & 35.R.P | 9566 | 586 | 587 | 588 | 589 | 590 | 591 | 592 | 593 |
| 4.I.G & 27.—.R | 9567 | 586 | 587 | 588 | 589 | 590 | 591 | 592 | 593 |
| 304.M.T & 398.Y.T | 585 | 586 | 587 | 9556 | 9558 | 590 | 591 | 592 | 593 |
| 304.M.T & 826.V.M | 585 | 586 | 587 | 9556 | 589 | 590 | 591 | 9562 | 593 |
| 304.M.T & 887.T.D | 585 | 586 | 587 | 9556 | 589 | 590 | 591 | 9560 | 593 |
| 304.M.T & 891.S.Q | 585 | 586 | 587 | 9556 | 589 | 590 | 591 | 9563 | 593 |
| 9.K.G & 35.R.P | 9568 | 586 | 587 | 588 | 589 | 590 | 591 | 592 | 593 |
| 5.—.G & 27.—.R | 9569 | 586 | 587 | 588 | 589 | 590 | 591 | 592 | 593 |
| 4.I.G & 9.K.G | 9570 | 586 | 587 | 588 | 589 | 590 | 591 | 592 | 593 |
| 398.Y.T & 826.V.M | 585 | 586 | 587 | 588 | 9558 | 590 | 591 | 9562 | 593 |
| 398.Y.T & 887.T.D | 585 | 586 | 587 | 588 | 9558 | 590 | 591 | 9560 | 593 |
| 398.Y.T & 891.S.Q | 585 | 586 | 587 | 588 | 9558 | 590 | 591 | 9563 | 593 |
| 27.—.R & 35.R.P | 9571 | 586 | 587 | 588 | 589 | 590 | 591 | 592 | 593 |
| 9.K.G & 27.—.R | 9572 | 586 | 587 | 588 | 589 | 590 | 591 | 592 | 593 |
| 5.—.G & 9.K.G | 9573 | 586 | 587 | 588 | 589 | 590 | 591 | 592 | 593 |
| 4.I.G & 5.—.G | 9574 | 586 | 587 | 588 | 589 | 590 | 591 | 592 | 593 |
| 826.V.M & 887.T.D | 585 | 586 | 587 | 588 | 589 | 590 | 591 | 9586 | 593 |
| 826.V.M & 891.S.Q | 585 | 586 | 587 | 588 | 589 | 590 | 591 | 9587 | 593 |
| 5.K.G & 27.—.R | 9575 | 586 | 587 | 588 | 589 | 590 | 591 | 592 | 593 |
| 5.K.G & 169.L.K | 9548 | 586 | 9553 | 588 | 589 | 590 | 591 | 592 | 593 |
| 5.K.G & 171.A.D | 9548 | 586 | 9545 | 588 | 589 | 590 | 591 | 592 | 593 |
| 5.K.G & 304.M.T | 9548 | 586 | 587 | 9556 | 589 | 590 | 591 | 592 | 593 |
| 5.K.G & 398.Y.T | 9548 | 586 | 587 | 588 | 9558 | 590 | 591 | 592 | 593 |
| 5.K.G & 891.S.Q | 9548 | 586 | 587 | 588 | 589 | 590 | 591 | 9563 | 593 |
| 6.—.G & 27.—.R | 9576 | 586 | 587 | 588 | 589 | 590 | 591 | 592 | 593 |
| 6.—.G & 169.L.K | 9549 | 586 | 9553 | 588 | 589 | 590 | 591 | 592 | 593 |
| 6.—.G & 171.A.D | 9549 | 586 | 9545 | 588 | 589 | 590 | 591 | 592 | 593 |
| 6.—.G & 304.M.T | 9549 | 586 | 587 | 9556 | 589 | 590 | 591 | 592 | 593 |
| 6.—.G & 398.Y.T | 9549 | 586 | 587 | 588 | 9558 | 590 | 591 | 592 | 593 |
| 6.—.G & 891.S.Q | 9549 | 586 | 587 | 588 | 589 | 590 | 591 | 9563 | 593 |
| 304.M.W & 27.—.R | 9543 | 586 | 587 | 9557 | 589 | 590 | 591 | 592 | 593 |
| 304.M.W & 169.L.K | 585 | 586 | 9553 | 9557 | 589 | 590 | 591 | 592 | 593 |
| 304.M.W & 171.A.D | 585 | 586 | 9545 | 9557 | 589 | 590 | 591 | 592 | 593 |
| 304.M.W & 398.Y.T | 585 | 586 | 587 | 9557 | 9558 | 590 | 591 | 592 | 593 |
| 304.M.W & 891.S.Q | 585 | 586 | 587 | 9557 | 589 | 590 | 591 | 9563 | 593 |
| 481.E.D & 27.—.R | 9543 | 586 | 587 | 588 | 9559 | 590 | 591 | 592 | 593 |
| 481.E.D & 169.L.K | 585 | 586 | 9553 | 588 | 9559 | 590 | 591 | 592 | 593 |
| 481.E.D & 171.A.D | 585 | 586 | 9545 | 588 | 9559 | 590 | 591 | 592 | 593 |
| 481.E.D & 304.M.T | 585 | 586 | 587 | 9556 | 9559 | 590 | 591 | 592 | 593 |
| 481.E.D & 398.Y.T | 585 | 586 | 587 | 588 | 9584 | 590 | 591 | 592 | 593 |
| 481.E.D & 891.S.Q | 585 | 586 | 587 | 588 | 9559 | 590 | 591 | 9563 | 593 |
| 698.S.R & 27.—.R | 9543 | 586 | 587 | 588 | 589 | 590 | 9564 | 592 | 593 |
| 698.S.R & 169.L.K | 585 | 586 | 9553 | 588 | 589 | 590 | 9564 | 592 | 593 |
| 698.S.R & 171.A.D | 585 | 586 | 9545 | 588 | 589 | 590 | 9564 | 592 | 593 |
| 698.S.R & 304.M.T | 585 | 586 | 587 | 9556 | 589 | 590 | 9564 | 592 | 593 |
| 698.S.R & 398.Y.T | 585 | 586 | 587 | 588 | 9558 | 590 | 9564 | 592 | 593 |
| 698.S.R & 891.S.Q | 585 | 586 | 587 | 588 | 589 | 590 | 9564 | 9563 | 593 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

A subset of these 161 CasX variants were cloned using methods standard in the art, and are listed in Tables 74, 76, and 77, below. In addition, a CasX variant termed CasX variant 1001 was generated by combining mutations from CasX variant 812 and CasX variant 676 (27.-.R, 169.L.K, and 329.G.K mutations relative to CasX 515), which have been previously validated as a highly specific and highly active CasX variants, respectively (the PAM-altering 224.G.S mutation also present in CasX 676 was not included). CasX variant 969 was generated by combining 27.-.R, 171.A.D, and 224.G.T mutations relative to CasX variant 515. Finally, CasX variant 973 was generated by combining 35.R.P, 171.A.Y, and 304.M.T mutations relative to CasX variant 515. The amino acid sequences of CasX variants 969, 973, and 1001 are provided in Table 73, below.

TABLE 73
Amino acid sequences of CasX variants 969, 973, and 1001

| CasX | | |
|---|---|---|
| variant | | SEQ |
| no. | Amino acid sequence | ID NO |
| 969 | QEIKRINKIRRRLVKDSNTKKAGKTRGPMKTLLVRVMTPDLRERLENLRKKPENIPQPIS | 9607 |
| | NTSRANLNKLLTDYTEMKKAILHVYWEEFQKDPVGLMSRVAQPASKKIDQNKLKPEMDEK | |
| | GNLTTAGFACSQCGQPLFVYKLEQVSEKGKAYTNYFGRCNVAEHEKLILLDQLKPEKDSD | |
| | EAVTYSLGKFGQRALDFYSIHVTKESTHPVKPLAQIAGNRYASTPVGKALSDACMGTIAS | |
| | FLSKYQDIIIEHQKVVKGNQKRLESLRELAGKENLEYPSVTLPPQPHTKEGVDAYNEVIA | |
| | RVRMWVNLNLWQKLKLSRDDAKPLLRLKGFPSFPLVERQANEVDWWDMVCNVKKLINEKK | |
| | EDGKVFWQNLAGYKRQEALRPYLSSEEDRKKGKKFARYQLGDLLLHLEKKHGEDWGKVYD | |
| | EAWERIDKKVEGLSKHIKLEEERRSEDAQSKAALTDWLRAKASFVIEGLKEADKDEFCRC | |
| | ELKLQKWYGDLRGKPFAIEAENSILDISGFSKQYNCAFIWQKDGVKKLNLYLIINYFKGG | |
| | KLRFKKIKPEAFEANRFYTVINKKSGEIVPMEVNFNFDDPNLIILPLAFGKRQGREFIWN | |
| | DLLSLETGSLKLANGRVIEKTLYNRRTRQDEPALFVALTFERREVLDSSNIKPMNLIGVD | |
| | RGENIPAVIALTDPEGCPLSRFKDSLGNPTHILRIGESYKEKQRTIQAKKEVEQRRAGGY | |
| | SRKYASKAKNLADDMVRNTARDLLYYAVTQDAMLIFENLSRGFGRQGKRTFMAERQYTRM | |
| | EDWLTAKLAYEGLPSKTYLSKTLAQYTSKTCSNCGFTITSADYDRVLEKLKKTATGWMTT | |
| | INGKELKVEGQITYYNRYKRQNVVKDLSVELDRLSEESVNNDISSWTKGRSGEALSLLKK | |
| | RFSHRPVQEKFVCLNCGFETHADEQAALNIARSWLFLRSQEYKKYQTNKTTGNTDKRAFV | |
| | ETWQSFYRKKLKEVWKPAV | |
| 973 | QEIKRINKIRRRLVKDSNTKKAGKTGPMKTLLVPVMTPDLRERLENLRKKPENIPQPISN | 9608 |
| | TSRANLNKLLTDYTEMKKAILHVYWEEFQKDPVGLMSRVAQPASKKIDQNKLKPEMDEKG | |
| | NLTTAGFACSQCGQPLFVYKLEQVSEKGKAYTNYFGRCNVAEHEKLILLYQLKPEKDSDE | |
| | AVTYSLGKFGQRALDFYSIHVTKESTHPVKPLAQIAGNRYASGPVGKALSDACMGTIASF | |
| | LSKYQDIIIEHQKVVKGNQKRLESLRELAGKENLEYPSVTLPPQPHTKEGVDAYNEVIAR | |
| | VRTWVNLNLWQKLKLSRDDAKPLLRLKGFPSFPLVERQANEVDWWDMVCNVKKLINEKKE | |
| | DGKVFWQNLAGYKRQEALRPYLSSEEDRKKGKKFARYQLGDLLLHLEKKHGEDWGKVYDE | |
| | AWERIDKKVEGLSKHIKLEEERRSEDAQSKAALTDWLRAKASFVIEGLKEADKDEFCRCE | |
| | LKLQKWYGDLRGKPFAIEAENSILDISGFSKQYNCAFIWQKDGVKKLNLYLIINYFKGGK | |
| | LRFKKIKPEAFEANRFYTVINKKSGEIVPMEVNFNFDDPNLIILPLAFGKRQGREFIWND | |
| | LLSLETGSLKLANGRVIEKTLYNRRTRQDEPALFVALTFERREVLDSSNIKPMNLIGVDR | |
| | GENIPAVIALTDPEGCPLSRFKDSLGNPTHILRIGESYKEKQRTIQAKKEVEQRRAGGYS | |
| | RKYASKAKNLADDMVRNTARDLLYYAVTQDAMLIFENLSRGFGRQGKRTFMAERQYTRME | |
| | DWLTAKLAYEGLPSKTYLSKTLAQYTSKTCSNCGFTITSADYDRVLEKLKKTATGWMTTI | |
| | NGKELKVEGQITYYNRYKRQNVVKDLSVELDRLSEESVNNDISSWTKGRSGEALSLLKKR | |
| | FSHRPVQEKFVCLNCGFETHADEQAALNIARSWLFLRSQEYKKYQTNKTTGNTDKRAFVE | |
| | TWQSFYRKKLKEVWKPAV | |
| 1001 | QEIKRINKIRRRLVKDSNTKKAGKTRGPMKTLLVRVMTPDLRERLENLRKKPENIPQPIS | 9609 |
| | NTSRANLNKLLTDYTEMKKAILHVYWEEFQKDPVGLMSRVAQPASKKIDQNKLKPEMDEK | |
| | GNLTTAGFACSQCGQPLFVYKLEQVSEKGKAYTNYFGRCNVAEHEKLIKLAQLKPEKDSD | |
| | EAVTYSLGKFGQRALDFYSIHVTKESTHPVKPLAQIAGNRYASGPVGKALSDACMGTIAS | |
| | FLSKYQDIIIEHQKVVKGNQKRLESLRELAGKENLEYPSVTLPPQPHTKEGVDAYNEVIA | |
| | RVRMWVNLNLWQKLKLSRDDAKPLLRLKKFPSFPLVERQANEVDWWDMVCNVKKLINEKK | |
| | EDGKVFWQNLAGYKRQEALRPYLSSEEDRKKGKKFARYQLGDLLLHLEKKHGEDWGKVYD | |
| | EAWERIDKKVEGLSKHIKLEEERRSEDAQSKAALTDWLRAKASFVIEGLKEADKDEFCRC | |
| | ELKLQKWYGDLRGKPFAIEAENSILDISGFSKQYNCAFIWQKDGVKKLNLYLIINYFKGG | |
| | KLRFKKIKPEAFEANRFYTVINKKSGEIVPMEVNFNFDDPNLIILPLAFGKRQGREFIWN | |
| | DLLSLETGSLKLANGRVIEKTLYNRRTRQDEPALFVALTFERREVLDSSNIKPMNLIGVD | |
| | RGENIPAVIALTDPEGCPLSRFKDSLGNPTHILRIGESYKEKQRTIQAKKEVEQRRAGGY | |
| | SRKYASKAKNLADDMVRNTARDLLYYAVTQDAMLIFENLSRGFGRQGKRTFMAERQYTRM | |
| | EDWLTAKLAYEGLPSKTYLSKTLAQYTSKTCSNCGFTITSADYDRVLEKLKKTATGWMTT | |
| | INGKELKVEGQITYYNRYKRQNVVKDLSVELDRLSEESVNNDISSWTKGRSGEALSLLKK | |
| | RFSHRPVQEKFVCLNCGFETHADEQAALNIARSWLFLRSQEYKKYQTNKTTGNTDKRAFV | |
| | ETWQSFYRKKLKEVWKPAV | |

A multiplexed pooled PASS assay was performed and analyzed as described in Example 37. As noted in Example 37, CasX variants were expressed using a relatively weakly-expressing promoter to reduce CasX protein expression and thereby improve the sensitivity of the assay. Samples were tested in duplicate, except for CasX variant 1006, which was tested in quadruplicate. In Tables 74, 76, 77, below, the results for the CasX variant 1006 samples are reported in two separate rows, each the average of two samples. Streptococcus pyogenes Cas9 without a guide RNA served as a negative control. CasX variant 515, CasX variant 676, and CasX variant 812 were also included as controls.

### Results

Table 74 provides the level of on-target editing produced by various CasX variants with mutations relative to CasX variant 515, ranked from highest to lowest activity.

TABLE 74
Average on-targeting editing activity of CasX variants, ranked from highest to lowest

| Protein | Mutation(s) relative | | | SEM on-target |
|---|---|---|---|---|
| Designation | to CasX 515* | Amino acid | Average on-target | TTC PAM |
| (CasX variant | (Position.Reference. | sequence | TTC PAM editing | editing activity |
| no., or Cas9) | Alternative) | SEQ ID NO | activity (fraction) | (fraction) |
| 1018 | 9.K.G & 891.S.Q | 9417 | 3.01E−01 | 1.20E−01 |
| 1007 | 304.M.T & 826.V.M | 9499 | 2.76E−01 | 7.86E−02 |
| 1006 | 826.V.M & 891.S.Q | 9513 | 2.38E−01 | 4.11E−02 |
| 987 | 169.L.K & 304.M.T | 9458 | 2.32E−01 | 3.38E−02 |
| 1014 | 4.I.G & 891.S.Q | 9393 | 2.29E−01 | 3.45E−02 |
| 1143 | 4.I.G & 826.V.M | 9391 | 2.29E−01 | 3.44E−02 |
| 1019 | 27.-.R & 171.A.S | 9423 | 2.22E−01 | 2.52E−02 |
| 1029 | 5.K.G & 891.S.Q | 9519 | 2.16E−01 | 2.96E−02 |
| 1006 | 826.V.M & 891.S.Q | 9513 | 2.16E−01 | 3.44E−02 |
| 1015 | 5.-.G & 304.M.T | 9401 | 2.11E−01 | 3.20E−02 |
| 970 | 27.-.R & 891.S.Q | 9429 | 2.09E−01 | 2.61E−02 |
| 1028 | 5.K.G & 304.M.T | 9517 | 2.07E−01 | 3.10E−02 |
| 996 | 171.A.Y & 891.S.Q | 9483 | 2.05E−01 | 3.42E−02 |
| 969 | 27.-.R & 171.A.D & 224.G.T | 9607 | 2.04E−01 | 2.64E−02 |
| 984 | 64.R.Q & 891.S.Q | 9453 | 2.03E−01 | 9.13E−02 |
| 1041 | 698.S.R & 891.S.Q | 9542 | 2.01E−01 | 3.17E−02 |
| 1016 | 9.K.G & 171.A.D | 9409 | 1.98E−01 | 3.20E−02 |
| 1000 | 224.G.T & 891.S.Q | 9495 | 1.92E−01 | 2.51E−02 |
| 999 | 224.G.T & 304.M.T | 9491 | 1.92E−01 | 2.90E−02 |
| 986 | 169.L.K & 171.A.S | 9456 | 1.90E−01 | 2.76E−02 |
| 977 | 64.R.Q & 169.L.K | 9443 | 1.90E−01 | 3.12E−02 |
| 792 | 27.-.R & 169.L.K | 9419 | 1.89E−01 | 2.89E−02 |
| 993 | 171.A.D & 224.G.T | 9472 | 1.88E−01 | 2.70E−02 |
| 997 | 171.A.S & 304.M.T | 9485 | 1.87E−01 | 2.83E−02 |
| 1025 | 169.L.K & 891.S.Q | 9462 | 1.87E−01 | 2.99E−02 |
| 1001 | 27.-.R & 169.L.K & 329.G.K | 9609 | 1.85E−01 | 2.57E−02 |
| 1040 | 481.E.D & 891.S.Q | 9536 | 1.85E−01 | 3.00E−02 |
| 1004 | 304.M.T & 891.S.Q | 9501 | 1.85E−01 | 3.11E−02 |
| 676 | 27.-.R & 170.L.K & 224.G.S | 355 | 1.84E−01 | 2.28E−02 |
| 1031 | 6.-.G & 169.L.K | 9521 | 1.84E−01 | 3.02E−02 |
| 980 | 64.R.Q & 171.A.S | 9447 | 1.80E−01 | 3.06E−02 |
| 981 | 64.R.Q & 304.M.T | 9449 | 1.79E−01 | 2.44E−02 |
| 985 | 169.L.K & 171.A.Y | 9455 | 1.73E−01 | 3.57E−02 |
| 989 | 169.L.Q & 224.G.T | 9466 | 1.73E−01 | 3.20E−02 |
| 992 | 169.L.Q & 887.T.D | 9470 | 1.70E−01 | 3.40E−02 |
| 994 | 171.A.Y & 224.G.T | 9478 | 1.68E−01 | 3.21E−02 |
| 1005 | 826.V.M & 887.T.D | 9512 | 1.68E−01 | 3.17E−02 |
| 983 | 64.R.Q & 887.T.D | 9452 | 1.68E−01 | 1.70E−02 |
| 1026 | 169.L.Q & 826.V.M | 9469 | 1.65E−01 | 3.09E−02 |
| 1009 | 4.I.G & 171.A.D | 9385 | 1.65E−01 | 6.43E−02 |
| 982 | 64.R.Q & 398.Y.T | 9450 | 1.64E−01 | 2.73E−02 |
| 978 | 64.R.Q & 169.L.Q | 9444 | 1.63E−01 | 2.96E−02 |
| 515 | — | 197 | 1.63E−01 | 2.38E−02 |
| 1003 | 9.K.G & 27.-.R | 9509 | 1.61E−01 | 2.28E−02 |
| 1017 | 9.K.G & 224.G.T | 9412 | 1.56E−01 | 2.73E−02 |
| 1020 | 35.R.P & 171.A.D | 9433 | 1.55E−01 | 3.01E−02 |
| 998 | 171.A.S & 826.V.M | 9487 | 1.54E−01 | 2.92E−02 |
| 1010 | 4.I.G & 171.A.Y | 9386 | 1.54E−01 | 2.71E−02 |
| 1022 | 35.R.P & 891.S.Q | 9441 | 1.48E−01 | 2.79E−02 |
| 1038 | 224.G.T & 826.V.M | 9493 | 1.48E−01 | 3.11E−02 |
| 1027 | 5.K.G & 171.A.D | 9516 | 1.42E−01 | 2.32E−02 |
| 1012 | 4.I.G & 398.Y.T | 9390 | 1.40E−01 | 2.10E−02 |
| 971 | 35.R.P & 169.L.Q | 9432 | 1.39E−01 | 2.75E−02 |
| 1032 | 6.-.G & 171.A.D | 9522 | 1.39E−01 | 2.40E−02 |
| 1024 | 169.L.K & 398.Y.T | 9459 | 1.38E−01 | 2.87E−02 |
| 1023 | 64.R.Q & 224.G.T | 9448 | 1.37E−01 | 2.34E−02 |
| 1036 | 171.A.S & 887.T.D | 9488 | 1.37E−01 | 2.38E−02 |
| 988 | 169.L.Q & 171.A.Y | 9464 | 1.36E−01 | 3.36E−02 |
| 1034 | 171.A.Y & 826.V.M | 9481 | 1.36E−01 | 3.05E−02 |
| 1030 | 171.A.D & 398.Y.T | 9474 | 1.35E−01 | 2.55E−02 |
| 1039 | 304.M.W & 398.Y.T | 9529 | 1.29E−01 | 3.28E−02 |
| 1033 | 171.A.Y & 304.M.T | 9479 | 1.25E−01 | 2.29E−02 |
| 1021 | 35.R.P & 304.M.T | 9437 | 1.25E−01 | 1.77E−02 |
| 1011 | 4.I.G & 224.G.T | 9388 | 1.24E−01 | 2.13E−02 |
| 979 | 64.R.Q & 171.A.Y | 9446 | 1.24E−01 | 2.15E−02 |
| 1002 | 5.-.G & 35.R.P | 9496 | 1.20E−01 | 2.31E−02 |
| 1035 | 171.A.S & 398.Y.T | 9486 | 1.14E−01 | 2.07E−02 |
| 851 | 35.R.P & 171.A.Y | 9434 | 1.13E−01 | 2.31E−02 |
| 995 | 171.A.Y & 398.Y.T | 9480 | 1.13E−01 | 1.83E−02 |
| 973 | 35.R.P & 171.A.Y & 304.M.T | 9608 | 1.12E−01 | 1.96E−02 |
| 976 | 35.R.P & 887.T.D | 9440 | 9.62E−02 | 2.52E−02 |
| 974 | 35.R.P & 224.G.T | 9436 | 9.00E−02 | 1.52E−02 |
| 1037 | 224.G.T & 398.Y.T | 9492 | 8.30E−02 | 1.35E−02 |
| 812 | 329.G.K | 484 | 7.68E−02 | 1.69E−02 |
| 975 | 35.R.P & 398.Y.T | 9438 | 7.49E−02 | 1.51E−02 |
| 991 | 169.L.Q & 398.Y.T | 9468 | 4.87E−02 | 1.74E−02 |
| Cas9 | n/a | — | 0.00E+00 | 0.00E+00 |

*Positions of mutations are shown relative to a CasX 515 sequence with an N-terminal methionine residue (SEQ ID NO: 9590). Each mutation is indicated by its position, reference sequence, and alt sequence, separated by ‘.’ Insertions are indicated with a ‘-’ in the reference sequence (first position), and deletions with a ‘-’ in the alt sequence (second position). Multiple individual mutations are separated by “&”.

As shown in Table 74, 41 of the tested CasX variants produced higher levels of on-target editing than did CasX variant 515; the 41 CasX variants are bolded in Table 74. CasX variants 1018 had 9.K.G and 891.S.Q amino acid substitutions and produced the highest level of on-target editing in the assay. The CasX variant 676 control was more active than CasX 515, and CasX variant 812 was less active than CasX variant 515, which is consistent with previous results.

A large number of the tested CasX variants produced lower levels of on-target editing than CasX variant 515. This suggests that not all combinations of mutations, including combinations of mutations that were relatively active for on-target editing when introduced into CasX variant 515 as single mutations (see Example 37), are compatible for producing highly active CasX variants.

To understand the amino acid residues that may be causal for improving CasX activity, the identity of the mutations in the CasX variants with two or three mutations resulting in improved on-target editing activity relative to CasX variant 515 was examined (Table 75).

TABLE 75
Summary of mutations in CasX variants with greater
on-target editing activity than CasX variant 515

| | Number of | |
|---|---|---|
| Position of mutation | occurrences of | Identity of mutation(s) |
| relative to CasX 515 | mutations at | (Number of CasX |
| (SEQ ID NO: 9590) | position* | variants with mutation) |
| 891 | 13 | 891.S.Q |
| 169 | 12 | 169.L.K (8); 169.L.Q (4) |
| 171 | 11 | 171.A.S (4); 171.A.D (4); |
| | | 171.A.Y (3) |
| 304 | 8 | 304.M.T |
| 64 | 7 | 64.R.Q |
| 224 | 6 | 224.G.T |
| 27 | 5 | 27.-.R |
| 826 | 4 | 826.V.M |
| 4 | 3 | 4.I.G |
| 5 | 3 | 5.-.G (1); 5.K.G (2) |
| 887 | 3 | 887.T.D |
| 9 | 2 | 9.K.G |
| 6 | 1 | 6.-.G |
| 481 | 1 | 481.E.D |
| 698 | 1 | 698.S.R |

*Excluding CasX variant 676.

As shown in Table 75, certain positions were mutated in several members of the set of CasX variants with higher on-target editing activity than CasX variant 515. For example, the serine to glutamine substitution at position 891 (891.S.Q), in the TSL domain, was found in 13 members of the CasX variants with improved on-target editing activity relative to CasX variant 515. The TSL domain is a dynamic domain involved in coordinating the introduction of the target strand to the RuvC active site, and the substitution of serine for the longer glutamine may allow for additional hydrogen bonding interactions with the target strand and more efficient transfer to the nuclease domain.

One of two substitutions at position 169 (169.L.K or 169.L.Q), in the NTSB domain, were found in 12 members of the CasX variants with higher on-target editing activity than CasX variant 515. This position is proximal to the second and third nucleotides of the unwound non-target strand in structures of the non-target strand loading state, and the introduction of either a charged residue or one capable of multiple hydrogen-bonding interactions likely allows for the stabilization of the unwound state and thus more efficient unwinding. It should be noted that 169.L.K was more enriched than 169.L.Q among the CasX variants with improved on-target editing activity, which suggests that while a polar interaction increases enzymatic activity, a charge-charge interaction is more suitable for this position.

One of three substitutions at position 171 (171.A.S, 171.A.D, or 171.A.Y), also in the NTSB domain, were found in 11 members of the CasX variants with improved on-target editing activity. Residue 171 is solvent-exposed, so a polar residue is likely more favorable at this position. While the residue is not in a position that interacts with the non-target strand in published structures, the dynamic nature of the NTSB domain may allow these residues to make hydrogen-bonding interactions with the target DNA at some point in the unwinding process. A serine is present at this position in the wild-type CasX 2 (SEQ ID NO: 2) sequence and is an alanine in CasX variants containing the chimeric NTSB from CasX1, meaning that the 171.A.S mutation in particular represents a reversion to a wild-type sequence. Notably, 171.A.Y was also found in several of the variants performing worse than CasX variant 515, which suggests that a tyrosine at position 171 might create too much steric hindrance for proper hydrogen-bonding interactions with the target DNA.

While the 169.L.K and 27.-.R mutations found in CasX variant 676 were well-represented among the high activity variants, there were a number of orthogonal mutations with distinct mechanisms that may allow for increased activity without the loss of specificity seen in CasX variant 676. 891.S.Q in particular was found in a number of top-performing activity variants that also have a higher specificity ratio than CasX variant 515 (see below).

Table 76, below, provides the level of off-target editing produced by various CasX variants with two or three mutations relative to CasX variant 515, ranked from lowest to highest activity.

TABLE 76
Average off-targeting editing activity of CasX variants, ranked from lowest to highest

| Protein | Mutation relative to | | | SEM off-target |
|---|---|---|---|---|
| Designation | CasX 515* | Amino acid | Average off-target | TTC PAM |
| (CasX variant | (Position.Reference. | sequence | TTC PAM editing | editing activity |
| no., or Cas9) | Alternative) | SEQ ID NO | activity (fraction) | (fraction) |
| Cas9 | n/a | — | 0.00E+00 | 0.00E+00 |
| 812 | 329.G.K | 484 | 4.46E−03 | 3.30E−03 |
| 991 | 169.L.Q & 398.Y.T | 9468 | 4.71E−03 | 3.59E−03 |
| 975 | 35.R.P & 398.Y.T | 9438 | 6.63E−03 | 5.75E−03 |
| 976 | 35.R.P & 887.T.D | 9440 | 8.76E−03 | 6.93E−03 |
| 851 | 35.R.P & 171.A.Y | 9434 | 9.95E−03 | 6.91E−03 |
| 974 | 35.R.P & 224.G.T | 9436 | 9.95E−03 | 6.79E−03 |
| 971 | 35.R.P & 169.L.Q | 9432 | 1.15E−02 | 8.07E−03 |
| 1037 | 224.G.T & 398.Y.T | 9492 | 1.22E−02 | 8.00E−03 |
| 1039 | 304.M.W & 398.Y.T | 9529 | 1.24E−02 | 1.01E−02 |
| 988 | 169.L.Q & 171.A.Y | 9464 | 1.30E−02 | 8.38E−03 |
| 973 | 35.R.P & 171.A.Y & 304.M.T | 9608 | 1.37E−02 | 9.35E−03 |
| 995 | 171.A.Y & 398.Y.T | 9480 | 1.53E−02 | 9.56E−03 |
| 1002 | 5.-.G & 35.R.P | 9496 | 1.69E−02 | 1.03E−02 |
| 1009 | 4.I.G & 171.A.D | 9385 | 1.76E−02 | 1.08E−02 |
| 1011 | 4.I.G & 224.G.T | 9388 | 1.80E−02 | 1.12E−02 |
| 1035 | 171.A.S & 398.Y.T | 9486 | 1.86E−02 | 9.86E−03 |
| 989 | 169.L.Q & 224.G.T | 9466 | 1.91E−02 | 1.19E−02 |
| 1018 | 9.K.G & 891.S.Q | 9417 | 1.91E−02 | 1.43E−02 |
| 1033 | 171.A.Y & 304.M.T | 9479 | 1.93E−02 | 1.09E−02 |
| 994 | 171.A.Y & 224.G.T | 9478 | 1.95E−02 | 1.21E−02 |
| 1020 | 35.R.P & 171.A.D | 9433 | 1.98E−02 | 1.24E−02 |
| 1022 | 35.R.P & 891.S.Q | 9441 | 2.04E−02 | 1.10E−02 |
| 979 | 64.R.Q & 171.A.Y | 9446 | 2.09E−02 | 1.24E−02 |
| 1032 | 6.-.G & 171.A.D | 9522 | 2.20E−02 | 1.15E−02 |
| 1027 | 5.K.G & 171.A.D | 9516 | 2.28E−02 | 1.29E−02 |
| 1041 | 698.S.R & 891.S.Q | 9542 | 2.41E−02 | 1.32E−02 |
| 1012 | 4.I.G & 398.Y.T | 9390 | 2.50E−02 | 1.30E−02 |
| 1030 | 171.A.D & 398.Y.T | 9474 | 2.53E−02 | 1.31E−02 |
| 1024 | 169.L.K & 398.Y.T | 9459 | 2.54E−02 | 1.39E−02 |
| 982 | 64.R.Q & 398.Y.T | 9450 | 2.58E−02 | 1.58E−02 |
| 983 | 64.R.Q & 887.T.D | 9452 | 2.59E−02 | 1.06E−02 |
| 1040 | 481.E.D & 891.S.Q | 9536 | 2.63E−02 | 1.30E−02 |
| 1017 | 9.K.G & 224.G.T | 9412 | 2.67E−02 | 1.64E−02 |
| 1001 | 27.-.R & 169.L.K & 329.G.K | 9609 | 2.74E−02 | 1.31E−02 |
| 992 | 169.L.Q & 887.T.D | 9470 | 2.78E−02 | 1.47E−02 |
| 1036 | 171.A.S & 887.T.D | 9488 | 2.80E−02 | 1.47E−02 |
| 1003 | 9.K.G & 27.-.R | 9509 | 2.80E−02 | 1.40E−02 |
| 1005 | 826.V.M & 887.T.D | 9512 | 2.81E−02 | 1.41E−02 |
| 978 | 64.R.Q & 169.L.Q | 9444 | 2.83E−02 | 1.48E−02 |
| 1021 | 35.R.P & 304.M.T | 9437 | 2.84E−02 | 1.62E−02 |
| 985 | 169.L.K & 171.A.Y | 9455 | 3.00E−02 | 1.62E−02 |
| 977 | 64.R.Q & 169.L.K | 9443 | 3.03E−02 | 1.46E−02 |
| 1023 | 64.R.Q & 224.G.T | 9448 | 3.05E−02 | 1.81E−02 |
| 1034 | 171.A.Y & 826.V.M | 9481 | 3.07E−02 | 1.24E−02 |
| 1010 | 4.I.G & 171.A.Y | 9386 | 3.17E−02 | 1.59E−02 |
| 1026 | 169.L.Q & 826.V.M | 9469 | 3.18E−02 | 1.44E−02 |
| 1016 | 9.K.G & 171.A.D | 9409 | 3.30E−02 | 1.43E−02 |
| 1038 | 224.G.T & 826.V.M | 9493 | 3.30E−02 | 1.58E−02 |
| 1029 | 5.K.G & 891.S.Q | 9519 | 3.31E−02 | 1.77E−02 |
| 1031 | 6.-.G & 169.L.K | 9521 | 3.38E−02 | 1.65E−02 |
| 980 | 64.R.Q & 171.A.S | 9447 | 3.41E−02 | 1.59E−02 |
| 993 | 171.A.D & 224.G.T | 9472 | 3.43E−02 | 1.68E−02 |
| 1028 | 5.K.G & 304.M.T | 9517 | 3.65E−02 | 1.85E−02 |
| 998 | 171.A.S & 826.V.M | 9487 | 3.68E−02 | 1.63E−02 |
| 515 | — | 197 | 3.83E−02 | 1.46E−02 |
| 999 | 224.G.T & 304.M.T | 9491 | 3.91E−02 | 1.93E−02 |
| 1000 | 224.G.T & 891.S.Q | 9495 | 4.14E−02 | 2.07E−02 |
| 996 | 171.A.Y & 891.S.Q | 9483 | 4.25E−02 | 2.02E−02 |
| 981 | 64.R.Q & 304.M.T | 9449 | 4.30E−02 | 2.20E−02 |
| 1014 | 4.I.G & 891.S.Q | 9393 | 4.63E−02 | 2.06E−02 |
| 986 | 169.L.K & 171.A.S | 9456 | 4.63E−02 | 2.46E−02 |
| 1006 | 826.V.M & 891.S.Q | 9513 | 4.66E−02 | 2.16E−02 |
| 1025 | 169.L.K & 891.S.Q | 9462 | 4.73E−02 | 2.04E−02 |
| 997 | 171.A.S & 304.M.T | 9485 | 4.82E−02 | 2.22E−02 |
| 1015 | 5.-.G & 304.M.T | 9401 | 5.10E−02 | 2.19E−02 |
| 987 | 169.L.K & 304.M.T | 9458 | 5.72E−02 | 2.55E−02 |
| 1006 | 826.V.M & 891.S.Q | 9513 | 5.87E−02 | 2.71E−02 |
| 969 | 27.-.R & 171.A.D & 224.G.T | 9607 | 5.88E−02 | 2.17E−02 |
| 1004 | 304.M.T & 891.S.Q | 9501 | 6.11E−02 | 2.26E−02 |
| 1143 | 4.I.G & 826.V.M | 9391 | 6.15E−02 | 2.79E−02 |
| 676 | 27.-.R & 170.L.K & 224.G.S | 355 | 6.19E−02 | 2.14E−02 |
| 1019 | 27.-.R & 171.A.S | 9423 | 6.78E−02 | 1.45E−02 |
| 970 | 27.-.R & 891.S.Q | 9429 | 6.87E−02 | 2.22E−02 |
| 1007 | 304.M.T & 826.V.M | 9499 | 6.99E−02 | 8.52E−02 |
| 984 | 64.R.Q & 891.S.Q | 9453 | 7.16E−02 | 1.01E−01 |
| 792 | 27.-.R & 169.L.K | 9419 | 7.26E−02 | 2.39E−02 |

As shown in Table 76, the majority of the tested CasX variants with pairs of mutations relative to CasX variant 515 produced lower levels of off-target editing than did CasX variant 515; these samples are bolded in Table 76.

Table 77, below, provides the specificity ratio (i.e., the average level of on-targeting editing divided by the average level of off-target editing) of the tested CasX variants with two or three mutations relative to CasX 515, ranked from the highest to lowest ratio. CasX variants with higher specificity ratios than CasX 515 are bolded in Table 77.

TABLE 77
Specificity ratios of CasX variants, ranked from highest to lowest*

| | | | | SEM off- |
|---|---|---|---|---|
| Protein | Mutation relative to | Amino acid | Specificity ratio | target TTC |
| Designation | CasX 515* | sequence | (average on-target | PAM editing |
| (CasX variant | (Position.Reference. | (SEQ ID | activity/average | activity |
| no., or Cas9) | Alternative) | NO) | off-target activity) | (fraction) |
| 812 | 329.G.K | 484 | 17.22 | 0.52 |
| 1018 | 9.K.G & 891.S.Q | 9417 | 15.76 | 0.35 |
| 971 | 35.R.P & 169.L.Q | 9432 | 12.09 | 0.5 |
| 851 | 35.R.P & 171.A.Y | 9434 | 11.36 | 0.49 |
| 975 | 35.R.P & 398.Y.T | 9438 | 11.3 | 0.67 |
| 976 | 35.R.P & 887.T.D | 9440 | 10.98 | 0.53 |
| 988 | 169.L.Q & 171.A.Y | 9464 | 10.46 | 0.4 |
| 1039 | 304.M.W & 398.Y.T | 9529 | 10.4 | 0.56 |
| 991 | 169.L.Q & 398.Y.T | 9468 | 10.34 | 0.41 |
| 1009 | 4.I.G & 171.A.D | 9385 | 9.38 | 0.23 |
| 989 | 169.L.Q & 224.G.T | 9466 | 9.06 | 0.44 |
| 974 | 35.R.P & 224.G.T | 9436 | 9.05 | 0.51 |
| 994 | 171.A.Y & 224.G.T | 9478 | 8.62 | 0.43 |
| 1041 | 698.S.R & 891.S.Q | 9542 | 8.34 | 0.39 |
| 973 | 35.R.P & 171.A.Y & 304.M.T | 9608 | 8.18 | 0.51 |
| 1020 | 35.R.P & 171.A.D | 9433 | 7.83 | 0.43 |
| 995 | 171.A.Y & 398.Y.T | 9480 | 7.39 | 0.46 |
| 1022 | 35.R.P & 891.S.Q | 9441 | 7.25 | 0.35 |
| 1002 | 5.-.G & 35.R.P | 9496 | 7.1 | 0.41 |
| 1040 | 481.E.D & 891.S.Q | 9536 | 7.03 | 0.33 |
| 1011 | 4.I.G & 224.G.T | 9388 | 6.89 | 0.45 |
| 1037 | 224.G.T & 398.Y.T | 9492 | 6.8 | 0.49 |
| 1001 | 27.-.R & 169.L.K & 329.G.K | 9609 | 6.75 | 0.34 |
| 1029 | 5.K.G & 891.S.Q | 9519 | 6.53 | 0.4 |
| 983 | 64.R.Q & 887.T.D | 9452 | 6.49 | 0.38 |
| 1033 | 171.A.Y & 304.M.T | 9479 | 6.48 | 0.31 |
| 982 | 64.R.Q & 398.Y.T | 9450 | 6.36 | 0.45 |
| 1032 | 6.-.G & 171.A.D | 9522 | 6.32 | 0.35 |
| 977 | 64.R.Q & 169.L.K | 9443 | 6.27 | 0.32 |
| 1027 | 5.K.G & 171.A.D | 9516 | 6.23 | 0.4 |
| 1035 | 171.A.S & 398.Y.T | 9486 | 6.13 | 0.35 |
| 992 | 169.L.Q & 887.T.D | 9470 | 6.12 | 0.33 |
| 1016 | 9.K.G & 171.A.D | 9409 | 6 | 0.27 |
| 1005 | 826.V.M & 887.T.D | 9512 | 5.98 | 0.31 |
| 979 | 64.R.Q & 171.A.Y | 9446 | 5.93 | 0.42 |
| 1017 | 9.K.G & 224.G.T | 9412 | 5.84 | 0.44 |
| 985 | 169.L.K & 171.A.Y | 9455 | 5.77 | 0.34 |
| 978 | 64.R.Q & 169.L.Q | 9444 | 5.76 | 0.33 |
| 1003 | 9.K.G & 27.-.R | 9509 | 5.75 | 0.36 |
| 1028 | 5.K.G & 304.M.T | 9517 | 5.67 | 0.36 |
| 1012 | 4.I.G & 398.Y.T | 9390 | 5.6 | 0.37 |
| 993 | 171.A.D & 224.G.T | 9472 | 5.48 | 0.34 |
| 1031 | 6.-.G & 169.L.K | 9521 | 5.44 | 0.34 |
| 1024 | 169.L.K & 398.Y.T | 9459 | 5.43 | 0.32 |
| 1030 | 171.A.D & 398.Y.T | 9474 | 5.34 | 0.33 |
| 980 | 64.R.Q & 171.A.S | 9447 | 5.28 | 0.3 |
| 1026 | 169.L.Q & 826.V.M | 9469 | 5.19 | 0.27 |
| 1006 | 826.V.M & 891.S.Q | 9513 | 5.11 | 0.29 |
| 1014 | 4.I.G & 891.S.Q | 9393 | 4.95 | 0.29 |
| 999 | 224.G.T & 304.M.T | 9491 | 4.91 | 0.34 |
| 1036 | 171.A.S & 887.T.D | 9488 | 4.89 | 0.35 |
| 1010 | 4.I.G & 171.A.Y | 9386 | 4.86 | 0.33 |
| 996 | 171.A.Y & 891.S.Q | 9483 | 4.82 | 0.31 |
| 1000 | 224.G.T & 891.S.Q | 9495 | 4.64 | 0.37 |
| 1023 | 64.R.Q & 224.G.T | 9448 | 4.49 | 0.42 |
| 1038 | 224.G.T & 826.V.M | 9493 | 4.48 | 0.27 |
| 1034 | 171.A.Y & 826.V.M | 9481 | 4.43 | 0.43 |
| 1021 | 35.R.P & 304.M.T | 9437 | 4.4 | 0.18 |
| 515 | — | 197 | 4.26 | 0.24 |
| 998 | 171.A.S & 826.V.M | 9487 | 4.18 | 0.25 |
| 981 | 64.R.Q & 304.M.T | 9449 | 4.16 | 0.37 |
| 1015 | 5.-.G & 304.M.T | 9401 | 4.14 | 0.28 |
| 986 | 169.L.K & 171.A.S | 9456 | 4.1 | 0.39 |
| 987 | 169.L.K & 304.M.T | 9458 | 4.06 | 0.3 |
| 1025 | 169.L.K & 891.S.Q | 9462 | 3.95 | 0.27 |
| 1007 | 304.M.T & 826.V.M | 9499 | 3.95 | 0.93 |
| 997 | 171.A.S & 304.M.T | 9485 | 3.88 | 0.31 |
| 1143 | 4.I.G & 826.V.M | 9391 | 3.72 | 0.3 |
| 1006 | 826.V.M & 891.S.Q | 9513 | 3.68 | 0.3 |
| 969 | 27.-.R & 171.A.D & 224.G.T | 9607 | 3.47 | 0.24 |
| 1019 | 27.-.R & 171.A.S | 9423 | 3.27 | 0.1 |
| 970 | 27.-.R & 891.S.Q | 9429 | 3.04 | 0.2 |
| 1004 | 304.M.T & 891.S.Q | 9501 | 3.03 | 0.2 |
| 676 | 27.-.R & 170.L.K & 224.G.S | 355 | 2.97 | 0.22 |
| 984 | 64.R.Q & 891.S.Q | 9453 | 2.84 | 0.95 |
| 792 | 27.-.R & 169.L.K | 9419 | 2.6 | 0.18 |

*Specificity ratio and SEM values are shown rounded to the nearest hundredth.

As shown in Table 77, the majority of the tested CasX variants had higher on-target to off-target editing ratios than CasX 515. While the previously validated high-specificity variant CasX\812 had the highest specificity ratio, many CasX variants demonstrated high specificity ratios without as significant a loss in on-target activity as was observed for CasX variant 812.

The 35.R.P mutation was commonly observed in variants with very high specificity ratios. This residue is in the OBD and believed to be involved in binding the guide RNA. Mutation to a proline at this position may have complex effects on allosteric regulation. Notably, these variants also tended to have low activity, suggesting that apparent specificity may be in part the result of less efficient RNP formation due to the disruption of this guide-binding interaction. Overall, an inverse correlation was observed between specificity ratio and activity. This suggests that it is difficult to fully avoid trade-offs between activity and specificity. However, it is also evident that the strategy of combining activity and specificity mutants can compensate for this trade-off and result in variants with both characteristics improved.

Notably, some CasX variants produced both higher levels of on-target editing and lower levels of off-target editing than did CasX 515, namely CasX variants 977, 978, 980, 982, 983, 985, 989, 992, 993, 994, 1001, 1005, 1009, 1016, 1018, 1026, 1028, 1029, 1031, 1040, and 1041. An even greater number had higher on-target activity and a higher specificity ratio, specifically, CasX variants 977, 978, 980, 982, 983, 985, 989, 992, 993, 994, 996, 999, 1000, 1001, 1005, 1006, 1009, 1014, 1016, 1018, 1026, 1028, 1029, 1031, 1040, and 1041. Such CasX variants are therefore interpreted to be highly active and highly specific.

Taken together, the results described herein demonstrate that mutations to CasX 515 can be introduced into the sequence that result in CasX variants with improved gene editing activity and/or specificity.

### Example 39: Generation and Assessment of Guide RNA Scaffolds with Mutations in the Pseudoknot Stem

As described in Example 18, guide RNA scaffold 320 was designed with mutations to deplete the CpG content of the DNA encoding the pseudoknot stem and the extended stem regions of the scaffold. In the experiment described in Example 18, scaffold 320 produced a significant increase in editing potency relative to scaffold 235. This suggested that mutations to the pseudoknot stem have the potential to improve scaffold function. In the following example, a selection of scaffolds with mutations in the pseudoknot stem were designed and tested for their ability to promote genome editing.

### Materials and Methods

Design of Guide RNA Scaffolds with Mutations in the Pseudoknot Stem:

Guide RNA scaffolds with mutations in the pseudoknot stem were designed based on scaffold 316 (SEQ ID NO: 9588). The positions of the mutations, as well as full-length DNA and RNA sequences of the scaffolds are provided in Table 78, below. Scaffold 392 recapitulates the CG->GC mutation in the pseudoknot stem that was used to generate scaffold 320, as described in Example 18. Scaffolds 174, 235, and 316 were included in this experiment as controls.

TABLE 78
Mutations and DNA and RNA sequences of guide RNA scaffolds

| | | | DNA | | RNA |
|---|---|---|---|---|---|
| Scaffold | Position of | | SEQ | | SEQ |
| no. | mutation* | DNA sequence | ID NO | RNA sequence | ID NO |
| 174 | n/a | ACTGGCGCTTTTATCTGA | 3631 | ACUGGCGCUUUUAUCUGAUUA | 2238 |
| | | TTACTTTGAGAGCCATCA | | CUUUGAGAGCCAUCACCAGCG | |
| | | CCAGCGACTATGTCGTAG | | ACUAUGUCGUAGUGGGUAAAG | |
| | | TGGGTAAAGCTCCCTCTT | | CUCCCUCUUCGGAGGGAGCAU | |
| | | CGGAGGGAGCATCAAAG | | CAAAG | |
| 235 | n/a | ACTGGCGCTTCTATCTGA | 3638 | ACUGGCGCUUCUAUCUGAUUA | 2292 |
| | | TTACTCTGAGCGCCATCA | | CUCUGAGCGCCAUCACCAGCG | |
| | | CCAGCGACTATGTCGTAG | | ACUAUGUCGUAGUGGGUAAAG | |
| | | TGGGTAAAGCCGCTTACG | | CCGCUUACGGACUUCGGUCCG | |
| | | GACTTCGGTCCGTAAGAG | | UAAGAGGCAUCAGAG | |
| | | GCATCAGAG | | | |
| 316 | n/a | ACTGGCGCTTCTATCTGA | 9335 | ACUGGCGCUUCUAUCUGAUUA | 9588 |
| | | TTACTCTGAGCGCCATCA | | CUCUGAGCGCCAUCACCAGCG | |
| | | CCAGCGACTATGTCGTAG | | ACUAUGUCGUAGUGGGUAAAG | |
| | | TGGGTAAAGCTCCCTCTT | | CUCCCUCUUCGGAGGGAGCAU | |
| | | CGGAGGGAGCATCAGAG | | CAGAG | |
| 320 | 5.CG.GC; | ACTGGGCCTTCTATCTGA | 3751 | ACUGGGCCUUCUAUCUGAUUA | 2376 |
| | 28.CG.GC; 64. | TTACTCTGAGGCCCATCA | | CUCUGAGGCCCAUCACCAGCG | |
| | TCCCTCTTC | CCAGCGACTATGTCGTAG | | ACUAUGUCGUAGUGGGUAAAG | |
| | GGAGGGA. | TGGGTAAAGCCGCTTAGG | | CCGCUUAGGGACUUCGGUCCC | |
| | CGCTTAGGG | GACTTCGGTCCCTAAGAG | | UAAGAGGCAUCAGAG | |
| | ACTTCGGTC | GCATCAGAG | | | |
| | CCTAAGAG | | | | |
| 332 | 5.CG.GC; | ACTGGGCCTTCTATCTGA | 3763 | ACUGGGCCUUCUAUCUGAUUA | 2391 |
| | 28.CG.GC; | TTACTCTGAGGCCCATCA | | CUCUGAGGCCCAUCACCAGCG | |
| | 74.-.A | CCAGCGACTATGTCGTAG | | ACUAUGUCGUAGUGGGUAAAG | |
| | | TGGGTAAAGCTCCCTCTT | | CUCCCUCUUCAGGAGGGAGCA | |
| | | CAGGAGGGAGCATCAGAG | | UCAGAG | |
| 376 | 6.-.T | ACTGGCTGCTTCTATCTG | 9610 | ACUGGCUGCUUCUAUCUGAUU | 9627 |
| | | ATTACTCTGAGCGCCATC | | ACUCUGAGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 377 | 6.-.G | ACTGGCGGCTTCTATCTG | 9611 | ACUGGCGGCUUCUAUCUGAUU | 9628 |
| | | ATTACTCTGAGCGCCATC | | ACUCUGAGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 378 | 6.-.G; 26.A.T | ACTGGCGGCTTCTATCTG | 9612 | ACUGGCGGCUUCUAUCUGAUU | 9629 |
| | | ATTACTCTGTGCGCCATC | | ACUCUGUGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 379 | 6.-.G; 27.-.G | ACTGGCGGCTTCTATCTG | 9613 | ACUGGCGGCUUCUAUCUGAUU | 9630 |
| | | ATTACTCTGAGGCGCCAT | | ACUCUGAGGCGCCAUCACCAG | |
| | | CACCAGCGACTATGTCGT | | CGACUAUGUCGUAGUGGGUAA | |
| | | AGTGGGTAAAGCTCCCTC | | AGCUCCCUCUUCGGAGGGAGC | |
| | | TTCGGAGGGAGCATCAGA | | AUCAGAG | |
| | | G | | | |
| 380 | 6.-.T; 28.-.C | ACTGGCTGCTTCTATCTG | 9614 | ACUGGCUGCUUCUAUCUGAUU | 9631 |
| | | ATTACTCTGAGCCGCCAT | | ACUCUGAGCCGCCAUCACCAG | |
| | | CACCAGCGACTATGTCGT | | CGACUAUGUCGUAGUGGGUAA | |
| | | AGTGGGTAAAGCTCCCTC | | AGCUCCCUCUUCGGAGGGAGC | |
| | | TTCGGAGGGAGCATCAGA | | AUCAGAG | |
| | | G | | | |
| 381 | 6.-.C; 27.-.G | ACTGGCCGCTTCTATCTG | 9615 | ACUGGCCGCUUCUAUCUGAUU | 9632 |
| | | ATTACTCTGAGGCGCCAT | | ACUCUGAGGCGCCAUCACCAG | |
| | | CACCAGCGACTATGTCGT | | CGACUAUGUCGUAGUGGGUAA | |
| | | AGTGGGTAAAGCTCCCTC | | AGCUCCCUCUUCGGAGGGAGC | |
| | | TTCGGAGGGAGCATCAGA | | AUCAGAG | |
| | | G | | | |
| 382 | 28.-.C | ACTGGCGCTTCTATCTGA | 9616 | ACUGGCGCUUCUAUCUGAUUA | 9633 |
| | | TTACTCTGAGCCGCCATC | | CUCUGAGCCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 383 | 28.-T | ACTGGCGCTTCTATCTGA | 9617 | ACUGGCGCUUCUAUCUGAUUA | 9634 |
| | | TTACTCTGAGTCGCCATC | | CUCUGAGUCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 384 | 6.-.G; 28.-.C | ACTGGCGGGTTCTATCTG | 9618 | ACUGGCGGGUUCUAUCUGAUU | 9635 |
| | | ATTACTCTGAGCCGCCAT | | ACUCUGAGCCGCCAUCACCAG | |
| | | CACCAGCGACTATGTCGT | | CGACUAUGUCGUAGUGGGUAA | |
| | | AGTGGGTAAAGCTCCCTC | | AGCUCCCUCUUCGGAGGGAGC | |
| | | TTCGGAGGGAGCATCAGA | | AUCAGAG | |
| | | G | | | |
| 385 | 6.-.C | ACTGGCCGCTTCTATCTG | 9619 | ACUGGCCGCUUCUAUCUGAUU | 9636 |
| | | ATTACTCTGAGCGCCATC | | ACUCUGAGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 386 | 5.C.T; 6.-.G | ACTGGTGGCTTCTATCTG | 9620 | ACUGGUGGCUUCUAUCUGAUU | 9637 |
| | | ATTACTCTGAGCGCCATC | | ACUCUGAGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 387 | 6.-.G | ACTGGCGGGTTCTATCTG | 9621 | ACUGGCGGGUUCUAUCUGAUU | 9638 |
| | | ATTACTCTGAGCGCCATC | | ACUCUGAGCGCCAUCACCAGC | |
| | | ACCAGCGACTATGTCGTA | | GACUAUGUCGUAGUGGGUAAA | |
| | | GTGGGTAAAGCTCCCTCT | | GCUCCCUCUUCGGAGGGAGCA | |
| | | TCGGAGGGAGCATCAGAG | | UCAGAG | |
| 388 | 6.-.G;28.-.C | ACTGGCGGCTTCTATCTG | 9622 | ACUGGCGGCUUCUAUCUGAUU | 9639 |
| | | ATTACTCTGGGCCGCCAT | | ACUCUGGGCCGCCAUCACCAG | |
| | | CACCAGCGACTATGTCGT | | CGACUAUGUCGUAGUGGGUAA | |
| | | AGTGGGTAAAGCTCCCTC | | AGCUCCCUCUUCGGAGGGAGC | |
| | | TTCGGAGGGAGCATCAGA | | AUCAGAG | |
| | | G | | | |
| 389 | 2.TGGC.CTG | ACCTGTAGGCTTCTATCT | 9623 | ACCUGUAGGCUUCUAUCUGAU | 9640 |
| | TAG; 26.- | GATTACTCTGCTACAGCG | | UACUCUGCUACAGCGCCAUCA | |
| | CTAC | CCATCACCAGCGACTATG | | CCAGCGACUAUGUCGUAGUGG | |
| | | TCGTAGTGGGTAAAGCTC | | GUAAAGCUCCCUCUUCGGAGG | |
| | | CCTCTTCGGAGGGAGCAT | | GAGCAUCAGAG | |
| | | CAGAG | | | |
| 390 | 2.TGGC.CAG | ACCAGCAAGCTTCTATCT | 9624 | ACCAGCAAGCUUCUAUCUGAU | 9641 |
| | CAA; 26.- | GATTACTCTGTTGCTGCG | | UACUCUGUUGCUGCGCCAUCA | |
| | .TTGC | CCATCACCAGCGACTATG | | CCAGCGACUAUGUCGUAGUGG | |
| | | TCGTAGTGGGTAAAGCTC | | GUAAAGCUCCCUCUUCGGAGG | |
| | | CCTCTTCGGAGGGAGCAT | | GAGCAUCAGAG | |
| | | CAGAG | | | |
| 391 | 2.TGGC.CGA | ACCGAGACGCTTCTATCT | 9625 | ACCGAGACGCUUCUAUCUGAU | 9642 |
| | GAC; 26.A.GG | GATTACTCTGGGCTCGCG | | UACUCUGGGCUCGCGCCAUCA | |
| | CTC | CCATCACCAGCGACTATG | | CCAGCGACUAUGUCGUAGUGG | |
| | | TCGTAGTGGGTAAAGCTC | | GUAAAGCUCCCUCUUCGGAGG | |
| | | CCTCTTCGGAGGGAGCAT | | GAGCAUCAGAG | |
| | | CAGAG | | | |
| 392 | 5.CG.GC;28.C | ACTGGGCCTTCTATCTGA | 9626 | ACUGGGCCUUCUAUCUGAUUA | 9643 |
| | G.GC | TTACTCTGAGGCCCATCA | | CUCUGAGGCCCAUCACCAGCG | |
| | | CCAGCGACTATGTCGTAG | | ACUAUGUCGUAGUGGGUAAAG | |
| | | TGGGTAAAGCCGCTTACG | | CCGCUUACGGACUUCGGUCCG | |
| | | GACTTCGGTCCGTAAGAG | | UAAGAGGCAUCAGAG | |
| | | GCATCAGAG | | | |

*positions are numbered relative to the 5′ end of scaffold 316, with “0” being the 5′ terminus. Each mutation is indicated by its position, reference sequence, and alt sequence, separated by ‘.’ (e.g., 1.C.A is nucleotide position 1, reference sequence nucleotide C, alternate sequence nucleotide A). Position indexing starts at 0 such that the first base in scaffold 316 is 0.A.
Insertions are indicated with a ‘—’ in the reference sequence, and deletions with a ′—’ in the alt sequence. Multiple individual mutations are semi-colon separated.

### Transfection and Assessment of B2M Editing:

HEK293T cells were lipofected with 100 ng of plasmid encoding CasX variant 515 and a gRNA made up of a scaffold listed in Table 78. The gRNAs had either a non-targeting spacer or a spacer targeting the B2M locus, as listed in Table 79. 24 hours post-transfection, cells were selected with 1 g/mL puromycin for 48 hours, and then allowed to recover for 24 hours. Then, cells were harvested for B2M protein expression analysis via immunostaining of the B2M-dependent HLA protein, followed by flow cytometry using the Attune™ NxT flow cytometer. Each construct was tested in duplicate, and the transfection and subsequent experiment was performed on two separate occasions.

TABLE 79
Sequences of B2M and non-targeting spacers used in this example

| Spacer ID | DNA sequence | SEQ ID NO |
|---|---|---|
| 7.9 | GTGTAGTACAAGAGATAGAA | 9644 |
| 7.19 | CCCCCACTGAAAAAGATGAG | 9645 |
| 7.43 | AGGCCAGAAAGAGAGAGTAG | 9646 |
| 7.119 | CGCTGGATAGCCTCCAGGCC | 9647 |
| 7.14 | TGAAGCTGACAGCATTCGGG | 9648 |
| Non-targeting | CGAGACGTAATTACGTCTCG | 9318 |

### Lentiviral Transduction and Assessment of B2M Editing:

In a separate experiment, HEK293T cells were transduced with lentiviral particles encoding CasX variant 515 and a gRNA made up of either scaffold 174, 235, 316, 382, or 392. The gRNAs had either a non-targeting spacer or spacer 7.9, 7.19, or 7.119 targeting the B2M locus, as provided in Table 79. Lentiviral particles were generated by transfecting Lenti-X HEK293T cells, seeded 24 hours prior, at a confluency of 70-90%. Plasmids containing the CasX and guide RNA expression cassettes are introduced to a second-generation lentiviral system containing the packaging and VSV-G envelope plasmids with polyethylenimine, in serum-free media. For particle production, media is changed 12 hours post-transfection, and viruses are harvested at 36-48 hours post-transfection. Viral supernatants were filtered using 0.45 μm membrane filters, diluted in media, and added to HEK293T target cells cultured at a relatively low multiplicity of infection (MOI) of either 0.1 or 0.05. Transduced cells were grown for three days in a 37PC incubator with 50 C02. Cells were harvested for B2M protein expression analysis via immunostaining of the B2M-dependent LA protein, followed by flow cytometry using the Attune™ NxT flow cytometer. The lentiviruses also expressed mScarlet, and the mean fluorescence intensity (MFI) of mScarlet was quantified to confirm that the cells contained similar amounts of transduced lentivirus.

### Results

Editing of the B2M locus was measured in HEK293T cells transfected with plasmids expressing CasX variant 515 and gRNAs made up of the scaffolds listed in Table 78. The results are provided in Table 80, below.

TABLE 80
Percentage of HEK293T cells with edited B2M locus following
transfection with plasmids expressing CasX 515 and gRNAs
with mutations to the scaffold pseudoknot stem

| | Percentage of HLA- HEK293T | |
|---|---|---|
| Scaffold | cells with edited B2M locus* | |
| no. | First transfection | Second transfection |

Non-targeting spacer

| | | | | |
|---|---|---|---|---|
| 174 | 24.3 | 22.5 | 4.2 | 5.2 |
| 235 | 1.7 | 1.4 | 3.5 | 4.3 |
| 316 | — | 4.1 | 3.1 | 3.9 |
| 316 | 1 | 3.8 | 3.6 | 4.5 |
| 320 | 1.4 | 1.4 | 2.5 | 3.1 |
| 332 | 1.6 | 1.6 | 2.5 | 3.6 |
| 376 | 3.2 | 4.9 | 4.3 | 5.4 |
| 377 | 0.6 | 5.6 | 5 | 6.3 |
| 378 | 0.8 | 4.8 | 4.3 | 6 |
| 379 | 1.3 | 5.6 | 5 | 6.7 |
| 380 | 1.3 | 4.8 | 4.7 | 5.1 |
| 381 | 1.3 | 5.1 | 4.6 | 7.3 |
| 381 | 6.9 | 5.9 | 4.3 | 7 |
| 382 | 1.2 | 4.5 | 3.8 | 6.4 |
| 383 | 1.2 | 3.7 | 3.1 | 5.1 |
| 384 | 1.6 | 5.3 | 2.4 | 5.3 |
| 385 | 1.4 | 1.2 | 3.3 | 3.5 |
| 386 | 1.5 | 1.2 | 2.3 | 2.7 |
| 387 | 1.5 | 1.1 | 3 | 3.7 |
| 388 | 2.7 | 1.7 | 5.5 | 6.3 |
| 389 | 1.6 | 1.2 | 2.4 | 3.1 |
| 390 | 1.3 | 1.3 | 2.4 | 3.1 |
| 391 | 1.4 | 1.3 | 2.6 | 3.4 |
| 392 | 1.6 | 1.4 | 3 | 4.2 |

Spacer 7.9

| | | | | |
|---|---|---|---|---|
| 174 | 68.5 | 62.1 | 85.4 | 80.6 |
| 235 | 73.1 | 68.3 | 91.8 | 88.9 |
| 316 | — | 85.5 | 82.7 | 84.7 |
| 316 | 72.6 | 92.2 | 91.4 | 92.2 |
| 320 | 75.4 | 70.4 | 92.6 | 91.2 |
| 332 | 70.5 | 63.5 | 85.2 | 84.4 |
| 376 | 67.8 | 90.6 | 89.9 | 90 |
| 377 | 71.3 | 90.9 | 90.4 | 90.3 |
| 378 | 75.5 | 90.8 | 91.2 | 92.2 |
| 379 | 68 | 87.9 | 87.6 | 87.4 |
| 380 | 65.2 | 90.5 | 90.6 | 90.8 |
| 381 | 67 | 88.7 | 88.7 | 89 |
| 381 | 61.6 | 86.9 | 88 | 88 |
| 382 | 71.9 | 89.1 | 90.5 | 90.6 |
| 383 | 64.7 | 91.6 | 91.1 | 91 |
| 384 | 62.5 | 79.7 | 82 | 83.1 |
| 385 | 68.4 | 62.5 | 83.2 | 81.1 |
| 386 | 72.5 | 63.1 | 90.7 | 91.1 |
| 387 | 74.5 | 69.4 | 94 | 92.9 |
| 388 | 67.8 | 65.1 | 89.9 | 90.1 |
| 389 | 32.2 | 28.9 | 54.1 | 53.8 |
| 390 | 34.5 | 30.9 | 57.8 | 57.7 |
| 391 | 25.9 | 25.7 | 55.2 | 55.4 |
| 392 | 77.1 | 71.5 | 93.5 | 91.6 |

Spacer 7.19

| | | | | |
|---|---|---|---|---|
| 174 | 58.6 | 47.5 | 81.4 | 81.7 |
| 235 | 62.5 | 54.2 | 90.3 | 89.8 |
| 316 | — | 82.6 | 78.8 | 83.5 |
| 316 | 63.3 | 82.9 | 86.4 | 86.9 |
| 320 | 67.4 | 60.3 | 91.7 | 92.1 |
| 332 | 1.8 | 1.5 | 4.8 | 7.1 |
| 376 | 52.5 | 80 | 78.8 | 80.2 |
| 377 | 56.6 | 83.4 | 84 | 83.4 |
| 378 | 57.1 | 83.8 | 82.9 | 84.2 |
| 379 | 49.2 | 72.4 | 75.8 | 76 |
| 380 | 45.6 | 73.6 | 74.4 | 76.1 |
| 381 | 49.2 | 70.7 | 74.7 | 76.2 |
| 381 | 43.5 | 73.5 | 75.5 | 78.3 |
| 382 | 59.7 | 81.9 | 84.7 | 86.6 |
| 383 | 50.4 | 78.7 | 78.9 | 80.6 |
| 384 | 45.7 | 78.9 | 76.6 | 77.5 |
| 385 | 54.3 | 48.9 | 72.7 | 71.6 |
| 386 | 56.3 | 47.6 | 76.5 | 77.7 |
| 387 | 64.5 | 59.1 | 86.9 | 89.7 |
| 388 | 48 | 45.3 | 75.5 | 77.6 |
| 389 | 3.1 | 2.6 | 8.9 | 12.9 |
| 390 | 18 | 15.4 | 36.4 | 38.9 |
| 391 | 14.7 | 12.8 | 38.5 | 40.3 |
| 392 | 64.3 | 58.4 | 87.6 | 87.8 |

Spacer 7.43

| | | | | |
|---|---|---|---|---|
| 174 | 50.6 | 43.9 | 76.2 | 75.4 |
| 235 | 58.7 | 52.5 | 80.4 | 80.2 |
| 316 | — | 68 | 67.1 | 70.5 |
| 316 | 52.2 | 78.2 | 77.7 | 78.4 |
| 320 | 59.6 | 53.2 | 83.9 | 82.1 |
| 332 | 49.3 | 40.5 | 69.9 | 67.6 |
| 376 | 56.6 | 80.2 | 81.6 | 81.3 |
| 377 | 64.7 | 81.2 | 84.1 | 83.2 |
| 378 | 56.5 | 76.8 | 79.6 | 78.8 |
| 379 | 59.5 | 77.1 | 79.2 | 78.9 |
| 380 | 58.3 | 77.1 | 78.9 | 78.1 |
| 381 | 50.9 | 71.4 | 74.2 | 70.1 |
| 381 | 47.5 | 66.6 | 70.7 | 65.5 |
| 382 | 57.1 | 75.3 | 80.7 | 79.8 |
| 383 | 52.9 | 72.5 | 75.3 | 73.4 |
| 384 | 46.6 | 71.6 | 74.6 | 70.7 |
| 385 | 56.7 | 46.9 | 70.9 | 73.4 |
| 386 | 62.5 | 48.2 | 80.5 | 82.2 |
| 387 | 56.8 | 49.3 | 81.8 | 82.6 |
| 388 | 53.5 | 48.9 | 75.1 | 77.2 |
| 389 | 4.5 | 4 | 13.6 | 18 |
| 390 | 15.1 | 11.7 | 29.5 | 34.5 |
| 391 | 16.2 | 13.4 | 33.6 | 38.7 |
| 392 | 53.8 | 51.8 | 82 | 82.1 |

Spacer 7.119

| | | | | |
|---|---|---|---|---|
| 174 | 36.2 | 32.2 | 64.6 | 63.6 |
| 235 | 60.9 | 55.5 | 89 | 87.3 |
| 316 | — | 66.5 | 68.5 | 70.6 |
| 316 | 56.4 | 73.7 | 79.5 | 83.3 |
| 320 | 54.6 | 46.8 | 85.3 | 83.9 |
| 332 | 51.7 | 46.1 | 74.8 | 78.2 |
| 376 | 45.8 | 66.9 | 71.8 | 75.1 |
| 377 | 49 | 69.4 | 76.3 | 79.2 |
| 378 | 45.7 | 67 | 75.1 | 78.1 |
| 379 | 35.2 | 49.9 | 59.3 | 61.8 |
| 380 | 37.9 | 56.2 | 66.2 | 67.3 |
| 381 | 31.9 | 50.2 | 52.4 | 56.8 |
| 381 | 28.9 | 46.2 | 50.2 | 52.5 |
| 382 | 49.8 | 67.8 | 71.3 | 76.3 |
| 383 | 44.2 | 60.9 | 65.5 | 69.6 |
| 384 | 36.8 | 59.3 | 64.9 | 66 |
| 385 | 39.1 | 32.8 | 62.8 | 62.7 |
| 386 | 40.9 | 33.8 | 72.7 | 74.5 |
| 387 | 7.8 | 6.2 | 24.1 | 31.4 |
| 388 | 35.8 | 30.6 | 62.2 | 64.4 |
| 389 | 4.9 | 3.9 | 15.3 | 20 |
| 390 | 4.3 | 3.9 | 11.9 | 17.1 |
| 391 | 3.8 | 3 | 9.3 | 12.9 |
| 392 | 58 | 50.2 | 85 | 83.3 |

Spacer 7.14

| | | | | |
|---|---|---|---|---|
| 174 | 47.5 | 43.7 | 74.1 | 76.4 |
| 235 | 31.4 | 29.6 | 58 | 57.2 |
| 316 | — | 44.8 | 41.6 | 40.3 |
| 316 | 31.8 | 52.7 | 53.2 | 55.6 |
| 320 | 36.4 | 34 | 59.9 | 62.3 |
| 332 | 14.8 | 12.4 | 25 | 25.4 |
| 376 | 34.1 | 55.5 | 56.1 | 56.9 |
| 377 | 24.6 | 45.1 | 42.7 | — |
| 378 | 26.9 | 45.1 | 43.5 | 47.5 |
| 379 | 36.1 | 54.8 | 56.2 | 59.3 |
| 380 | 46.6 | 67.4 | 70.2 | 72.3 |
| 381 | 50.8 | 72.1 | 73.4 | 74 |
| 381 | — | 39.3 | 35.2 | 39.4 |
| 382 | 42.2 | 65.6 | 66.4 | 67 |
| 383 | 37 | 64 | 64.9 | 64.9 |
| 384 | 18 | 38.4 | 35.1 | 38.1 |
| 385 | 18.1 | 16.1 | 29.6 | 24 |
| 386 | 36.2 | 31.6 | 53.1 | 53.8 |
| 387 | 33.3 | 28.2 | 48.1 | 51 |
| 388 | 31 | 25.5 | 49.3 | 55.4 |
| 389 | 20.6 | 17.6 | 45 | 45.8 |
| 390 | 8.4 | 7.9 | 19.3 | 22.6 |
| 391 | 52.8 | 52.9 | 80.9 | 78.9 |
| 392 | 42.3 | 42.6 | 66.7 | 67.2 |

*Data are shown rounded to the nearest tenth.

Many of the tested scaffolds produced levels of editing that were similar to scaffold 316 and higher than scaffold 174 (Table 80). Surprisingly, some scaffolds produced higher levels of editing than scaffold 316, but only with certain spacers. Specifically, scaffold 391 showed a relatively high level of editing with spacer 7.14, but not other spacers. Scaffold 392 produced overall high levels of editing with multiple spacers, and edited with spacer 7.14 to a greater extent than scaffold 316.

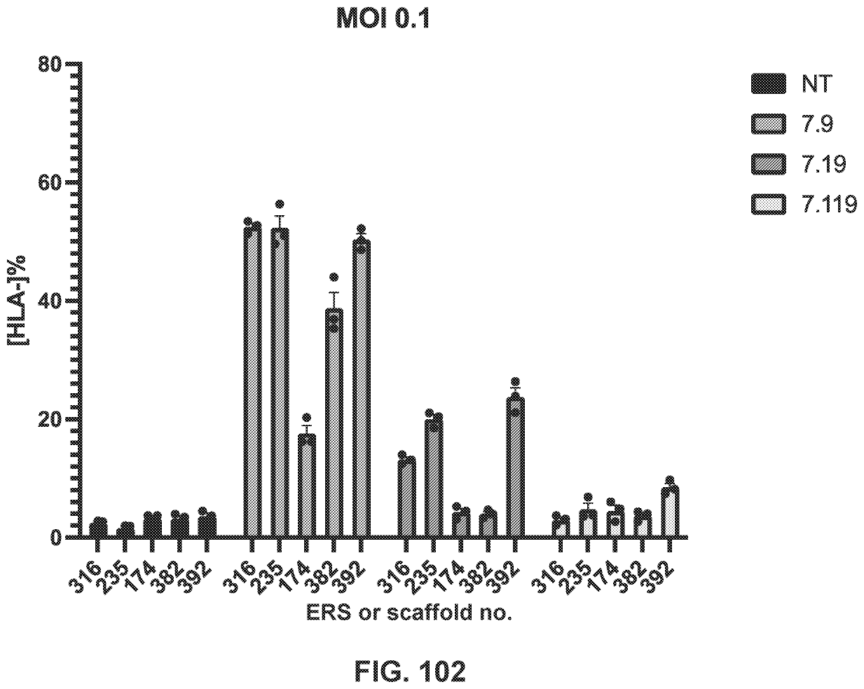

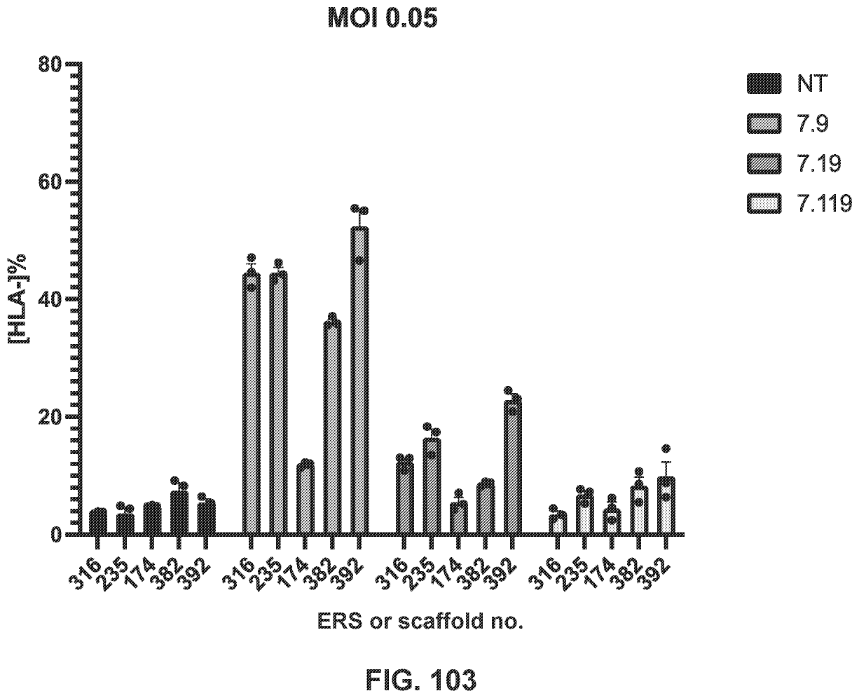

Scaffolds 174, 235, 316, 382, and 392 were also tested via lentiviral transduction in HEK293T cells at MOIs of 0.1 (FIG. 102) and 0.05 (FIG. 103). At these relatively low MOIs, the improvement in editing activity in scaffold 235 and scaffold 316 relative to scaffold 174 was pronounced, with both scaffold 235 and scaffold 316 producing over twice as many cells with edited B2M loci as scaffold 174. These results show that scaffold 235 and scaffold 316 are highly effective scaffolds for producing gene editing at low doses in cell culture, and are therefore also expected to be highly useful scaffolds for editing in vivo. In these assays, scaffold 392 produced similar levels of editing to scaffold 316 with the tested spacers.

Overall, the results described herein demonstrate that guide RNA scaffolds with mutations in the pseudoknot stem region can produce gene editing.

1. A recombinant adeno-associated virus (rAAV) transgene wherein
a. the transgene comprises:
i) a polynucleotide sequence encoding a CasX protein comprising a sequence selected from the group consisting of SEQ ID NOS: 137-512, 9382-9542, and 9607-9609, or a sequence having at least about 70% sequence identity thereto; and
ii) a polynucleotide sequence encoding a first guide RNA (gRNA) comprising a targeting sequence of 15 to 20 nucleotides complementary to a target nucleic acid of a cell;
b. the transgene has less than about 4700 nucleotides; and
c. the rAAV transgene is configured for incorporation into a rAAV capsid.

2. The rAAV transgene of claim 1, wherein the CasX protein comprises a sequence selected from the group consisting of SEQ ID NOS: 137-512, 9382-9542 and 9607-9609.

3. The rAAV of claim 1 or claim 2, wherein the wherein the CasX protein is a CasX variant selected from the group consisting of SEQ ID NOS: 9385, 9391, 9393, 9401, 9409, 9417, 9419, 9423, 9429, 9443, 9444, 9447, 9449, 9450, 9452, 9453, 9455, 9456, 9458, 9462, 9466, 9469, 9470, 9472, 9478, 9483, 9485, 9491, 9495, 9499, 9501, 9512, 9513, 9517, 9519, 9521, 9536, 9542, 9607, and 9609, wherein the encoded CasX variant exhibits improved editing of a target nucleic acid in an in vitro assay, compared to a CasX variant of SEQ ID NO: 197 and assayed under comparable conditions.

4. The rAAV of claim 1 or claim 2, wherein the wherein the CasX protein is a CasX variant selected from the group consisting of SEQ ID NOS: 9385, 9386, 9388, 9390, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9486, 9487, 9488, 9492, 9493, 9496, 9509, 9512, 9516, 9517, 9519, 9521, 9522, 9529, 9536, 9542, 9608, and 9609, wherein the encoded CasX variant exhibits improved editing specificity of a target nucleic acid in an in vitro assay, compared to a CasX variant of SEQ ID NO: 197 and assayed under comparable conditions.

5. The rAAV of claim 1 or claim 2, wherein the wherein the CasX protein is a CasX variant selected from the group consisting of SEQ ID NOS: 9385, 9386, 9388, 9390, 9393, 9409, 9412, 9417, 9432, 9433, 9434, 9436, 9437, 9438, 9440, 9441, 9443, 9444, 9446, 9447, 9448, 9450, 9452, 9455, 9459, 9464, 9466, 9468, 9469, 9470, 9472, 9474, 9478, 9479, 9480, 9481, 9483, 9486, 9488, 9491, 9492, 9493, 9495, 9496, 9509, 9512, 9513, 9516, 9517, 9519, 9521, 9522, 9529, 9536, 9542, 9608, and 9609, wherein the encoded CasX variant exhibits improved editing specificity ratio of a target nucleic acid in an in vitro assay, compared to a CasX variant of SEQ ID NO: 197 and assayed under comparable conditions.

6. The rAAV of claim 1 or claim 2, wherein the wherein the CasX protein is a CasX variant selected from the group consisting of SEQ ID NOS: 9385, 9409, 9417, 9443, 9444, 9447, 9450, 9452, 9455, 9466, 9469, 9470, 9472, 9478, 9512, 9513, 9517, 9519, 9521, 9536, 9542, and 9609, wherein the encoded CasX variant exhibits improved editing and improved specificity of a target nucleic acid in an in vitro assay, compared to a CasX variant of SEQ ID NO: 197 and assayed under comparable conditions.

7. The rAAV of claim 1 or claim 2, wherein the wherein the CasX protein is a CasX variant selected from the group consisting of SEQ ID NOS: 9385, 9393, 9409, 9417, 9443, 9444, 9447, 9450, 9452, 9455, 9466, 9469, 9470, 9472, 9478, 9483, 9491, 9495, 9512, 9513, 9517, 9519, 9521, 9536, 9542, and 9609, wherein the encoded CasX variant exhibits improved editing and improved specificity ratio of a target nucleic acid in an in vitro assay, compared to a CasX variant of SEQ ID NO: 197 and assayed under comparable conditions.

8. The rAAV transgene of claim 2, wherein the CasX protein comprises a sequence selected from the group consisting of SEQ ID NOS: 190 and 197.

9. The rAAV transgene of any one of claims 1-7, wherein the transgene further comprises one or more components selected from the group consisting of:
a. a first and a second rAAV inverted terminal repeat (ITR) sequence;
b. a first promoter sequence operably linked to the Type V CRISPR protein;
c. a sequence encoding a nuclear localization signal (NLS);
d. a 3′ UTR;
e. a poly(A) signal sequence;
f. a second promoter operably linked to the first gRNA; and
g. an accessory element.

10. The rAAV transgene of claim 9, wherein the first promoter is a pol II promoter selected from the group consisting of polyubiquitin C (UBC) promoter, cytomegalovirus (CMV) promoter, simian virus 40 (SV40) promoter, chicken beta-Actin promoter and rabbit beta-Globin splice acceptor site fusion (CAG), chicken β-actin promoter with cytomegalovirus enhancer (CB7), PGK promoter, Jens Tornoe (JeT) promoter, GUSB promoter, CBA hybrid (CBh) promoter, elongation factor-1 alpha (EF-1alpha) promoter, beta-actin promoter, Rous sarcoma virus (RSV) promoter, silencing-prone spleen focus forming virus (SFFV) promoter, CMVd1 promoter, truncated human CMV (tCMVd2) promoter, minimal CMV promoter, hepB promoter, chicken j-actin promoter, HSV TK promoter, Mini-TK promoter, minimal IL-2 promoter, GRP94 promoter, Super Core Promoter 1, Super Core Promoter 2, Super Core Promoter 3, adenovirus major late (AdML) promoter, MLC promoter, MCK promoter, GRK1 protein promoter, Rho promoter, CAR protein promoter, hSyn Promoter, Ula promoter, Ribosomal Protein Large subunit 30 (Rpl30) promoter, Ribosomal Protein Small subunit 18 (Rps18) promoter, CMV53 promoter, minimal SV40 promoter, CMV53 promoter, SFCp promoter, Mecp2 promoter, pJB42CAT5 promoter, MLP promoter, EFS promoter, rhodopsin promoter, MeP426 promoter, MecP2 promoter, Desmin promoter, MHCK promoter, MHCK7 promoter, beta-glucuronidase (GUSB) promoter, CK7 promoter, and CK8e promoter.

11. The rAAV transgene of claim 9 or claim 10, wherein the first promoter is a pol II promoter selected from the group consisting of U1A, UbC, and JeT.

12. The rAAV transgene of any one of claims 9-11, wherein the first promoter comprises a sequence selected from the group consisting of SEQ ID NOS: 3532-3562, 3714-3739, 3773-3778, and 9344-9350, or a sequence having at least about 70%, at least about 80%, at least about 85%, at least about 90%, at least about 95%, at least about 95%, at least about 96%, at least about 97%, at least about 98%, or at least about 99% identity thereto.

13. The polynucleotide of any one of claims 9-12, wherein the first promoter sequence has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides.

14. The rAAV transgene of any one of claims 9-13, wherein the second promoter is a pol III promoter selected from the group consisting of human U6 promoter, human U6 variant promoter, human U6 isoform variant promoter, mini U61 promoter, mini U62 promoter, mini U63 promoter, BiH1 (Bidrectional H1 promoter), BiU6 (Bidirectional U6 promoter), gorilla U6 promoter, rhesus U6 promoter, human 7sk promoter, and human HI promoter.

15. The rAAV transgene of claim 14, wherein the second promoter is a pol III promoter selected from the group consisting of human U6, human U6 variant, or human U6 isoform variant.

16. The rAAV transgene of claim 15, wherein the second promoter comprises a sequence selected from the group consisting of SEQ ID NOS: 3563, 3566-3582, 3599-3602, 3740-3746, 4025, 4029, 4032, and 4743 or a sequence having at least about 70%, at least about 80%, at least about 85%, at least about 90%, at least about 95%, at least about 95%, at least about 96%, at least about 97%, at least about 98%, or at least about 99% identity thereto.

17. The rAAV transgene of any one of claims 14-16, wherein the second promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

18. The rAAV transgene of any one of claims 9-17, wherein the poly(A) signal sequence is selected from the group consisting of SEQ ID NOS: 2401-3401, or a sequence having at least about 70%, at least about 80%, at least about 85%, at least about 90%, at least about 95%, at least about 95%, at least about 96%, at least about 97%, at least about 98%, or at least about 99% identity thereto.

19. The rAAV transgene of any one of claims 9-18, wherein the encoded NLS comprises a sequence selected from the group consisting of SEQ ID NOS: 3411-3486, 3939-3971, and 4065-4111.

20. The rAAV transgene of any one of claims 1-19, wherein the transgene comprises a polynucleotide sequence encoding a second gRNA with a linked targeting sequence of 15 to 20 nucleotides complementary to a different or overlapping region of a target nucleic acid of a cell, as compared to the targeting sequence of the first gRNA.

21. The rAAV transgene of any one of claims 1-20, wherein the first and/or the second gRNA each comprise:
a. a scaffold sequence selected from the group consisting of SEQ ID NOS: 2238-2400, 9257-9289 and 9588, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto; or
b. a scaffold sequence selected from the group consisting of SEQ ID NOS: 2238-2400, 9257-9289 and 9588, further comprising at least 1, 2, 3, 4, or 5 mismatches thereto.

22. The rAAV transgene of claim 20 or claim 21, wherein the first and the second gRNA each comprise a scaffold sequence of SEQ ID NO: 2293 or SEQ ID NO: 9588.

23. The rAAV transgene of any one of claims 20-22, comprising a third promoter operably linked to the second gRNA.

24. The rAAV transgene of claim 23, wherein the third promoter is a pol III promoter selected from the group consisting of human U6, human U6 variant, human U6 isoform variant, mini U61, mini U62, mini U63, BiH1 (Bidirectional H1 promoter), BiU6 (Bidirectional U6 promoter), gorilla U6, rhesus U6, human 7sk, and human H1 promoters.

25. The rAAV transgene of claim 23, wherein the third promoter is a pol III promoter selected from the group consisting of human U6, human U6 variant, and human U6 isoform variant.

26. The rAAV transgene of claim any one of claims 23-25, wherein the third promoter comprises a sequence selected from the group consisting of SEQ ID NOS: 3563, 3566-3582, 3599-3602, 3740-3746, 4025, 4029, 4032, and 4743, or a sequence having at least about 70%, at least about 80%, at least about 85%, at least about 90%, at least about 95%, at least about 95%, at least about 96%, at least about 97%, at least about 98%, or at least about 99% identity thereto.

27. The rAAV transgene of any one of claims 23-26, wherein the third promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

28. The rAAV transgene of any one of claims 20-27, wherein:
a. the polynucleotide sequence encoding the first gRNA and the polynucleotide sequence encoding the second gRNA are 5′ of the polynucleotide sequence encoding the CasX protein;
b. the polynucleotide sequence encoding the first gRNA is 5′ of the polynucleotide sequence encoding the CasX protein and the polynucleotide sequence encoding the second gRNA is 3′ of the polynucleotide sequence encoding the CasX protein;
c. the polynucleotide sequence encoding the first gRNA is 3′ of the polynucleotide sequence encoding the CasX protein and the polynucleotide sequence encoding the second gRNA is 5′ of the polynucleotide sequence encoding the CasX protein; or
d. the polynucleotide sequence encoding the first gRNA and the polynucleotide sequence encoding the second gRNA are 3′ of the polynucleotide sequence encoding the CasX protein.

29. The rAAV transgene of any one of claims 20-28, wherein:
a. the polynucleotide sequence encoding the first gRNA and the polynucleotide sequence encoding the second gRNA are encoded in a forward orientation relative to the polynucleotide sequence encoding the CasX protein;
b. the polynucleotide sequence encoding the first gRNA is encoded in a forward orientation relative to the polynucleotide sequence encoding the CasX protein and the polynucleotide sequence encoding the second gRNA is encoded in a reverse orientation relative to the polynucleotide sequence encoding the CasX protein;
c. the polynucleotide sequence encoding the first gRNA is encoded in a reverse orientation relative to the polynucleotide sequence encoding the CasX protein and the polynucleotide sequence encoding the second gRNA is encoded in a forward orientation relative to the polynucleotide sequence encoding the CasX protein; or
d. the polynucleotide sequence encoding the first gRNA and the polynucleotide sequence encoding the second gRNA are encoded in a reverse orientation relative to the polynucleotide sequence encoding the CasX protein.

30. The rAAV transgene of any one of claims 20-29, wherein the transgene has less than about 4800, less than about 4750, less than about 4700, less than about 4650 nucleotides, or less than about 4600 nucleotides.

31. The rAAV transgene of any one of claims 20-30, wherein the rAAV transgene is configured for incorporation into an rAAV capsid.

32. The rAAV transgene of any one of claims 1-31, wherein one or more components of the transgene are optimized to reduce or deplete CpG motifs.

33. The rAAV transgene of claim 32, wherein the one or more components comprise less than about 10%, less than about 5%, or less than about 1% CpG dinucleotides.

34. The rAAV transgene of claim 32 or claim 33, wherein the CpG-depleted polynucleotide sequence encoding the CasX protein is selected from the group consisting of SEQ ID NOS: 9327-9333 and 9369-9380.

35. The rAAV transgene of claim 32 or claim 33, wherein the CpG-depleted polynucleotide sequence encodes a gRNA scaffold, and is selected from the group consisting of SEQ ID NOS: 3751-3772.

36. The rAAV transgene of claim 32 or claim 33, wherein the CpG-depleted polynucleotide sequence of the ITR is selected from the group consisting of SEQ ID NOS: 3749 and 3750.

37. The rAAV transgene of claim 32 or claim 33, wherein the CpG-depleted polynucleotide sequence of the promoter is selected from the group consisting of SEQ ID NOS: 3735-3746.

38. The rAAV transgene of claim 32 or claim 33, wherein the CpG-depleted polynucleotide sequence of the poly(A) signal is SEQ ID NO: 3748.

39. The rAAV transgene of any one of claims 1-38, wherein the transgene has the configuration of a construct depicted in any one of FIGS. 1, 25, 28, 38-40, 47 and 75.

40. A recombinant adeno-associated virus (rAAV) comprising:
a. an AAV capsid protein, and
b. the transgene of any one of claims 1-39.

41. The rAAV of claim 40, wherein the AAV capsid protein is derived from serotype AAV1, AAV2, AAV3, AAV4, AAV5, AAV6, AAV7, AAV8, AAV9, AAV10, AAV 11, AAV12, AAV 9.45, AAV 9.61, AAV 44.9, AAV-Rh74, AAVRh10, MyoAAV 1Al, MyoAAV 1A2, or MyoAAV 2A.

42. The rAAV of claim 41, wherein the AAV capsid protein and the 5′ and 3′ ITR are derived from the same serotype of AAV.

43. The rAAV of claim 41, wherein the AAV capsid protein and the 5′ and 3′ ITR are derived from different serotypes of AAV.

44. The rAAV of claim 43, wherein the 5′ and 3′ ITR are derived from AAV serotype 2.

45. The rAAV of any one of claims 40-44, wherein upon transduction of a cell with the rAAV, the CasX protein and the first and/or the second gRNA encoded in the rAAV transgene are expressed.

46. The rAAV of claim 45, wherein upon expression, the first and/or the second gRNA is capable of forming a ribonucleoprotein (RNP) complex with the CasX protein.

47. The rAAV of claim 46, wherein the RNP is capable of binding and modifying a target nucleic acid of the cell.

48. The rAAV of any one of claims 40-47, wherein inclusion of a poly(A) signal in the transgene enhances expression of the CasX protein and editing efficiency of a target nucleic acid in a cell transduced by the rAAV.

49. The rAAV of any one of claims 40-48, wherein inclusion of a posttranscriptional regulatory element (PTRE) accessory element in the transgene enhances editing efficiency of a target nucleic acid in a cell transduced by the rAAV.

50. The rAAV of claim 49, wherein the PTRE comprises a sequence selected from the group consisting of SEQ ID NOS: 3615-3617, or a sequence having at least about 70%, at least about 80%, at least about 85%, at least about 90%, at least about 95%, at least about 95%, at least about 96%, at least about 97%, at least about 98%, or at least about 99% identity thereto.

51. The rAAV of any one of claims 40-50, wherein components of the transgene modified for depletion of all or a portion of the CpG dinucleotides exhibit a lower potential for inducing an immune response in a cell transduced with the rAAV, compared to a rAAV wherein the components are not modified for depletion of the CpG dinucleotides.

52. The rAAV of claim 51, wherein the lower potential for inducing an immune response is exhibited in an in vitro mammalian cell assay designed to detect production of one or more markers of an inflammatory response selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF).

53. The rAAV of claim 51 or claim 52, wherein the rAAV comprising the component sequences modified for depletion of all or a portion of the CpG dinucleotides elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

54. The rAAV of any one of claims 51-53, wherein the expressed CasX and the first and/or the second gRNA retain at least about 60%, at least about 70%, at least about 80%, or at least about 90% of the editing potential for a target nucleic acid compared to an rAAV wherein the transgene has not been optimized for depletion of CpG dinucleotides, when assayed in an in vitro assay under comparable conditions.

55. The rAAV of claim 40, wherein incorporation of a Pol II promoter selected from the group consisting of CK8e, MHCK7, and MHCK in the transgene of a rAAV used to transduce a muscle cell results in higher expression of the CasX protein in the muscle cell compared to incorporation of a UbC promoter.

56. The rAAV of claim 40, wherein incorporation of a muscle enhancer sequence selected from the group consisting of SEQ ID NOS: 3779-3809 in the transgene of a rAAV used to transduce a muscle cell results in higher expression of the CasX protein in the muscle cell compared to a rAAV not incorporating the muscle enhancer.

57. A method for modifying a target nucleic acid of a gene in a population of mammalian cells, comprising contacting a plurality of the cells with an effective amount of the rAAV of any one of claims 40-5656, wherein the target nucleic acid of the gene targeted by the first and/or the second gRNA is modified by the expressed CasX protein.

58. The method of claim 57, wherein the gene comprises one or more mutations.

59. The method of claim 57 or claim 58, wherein the modifying comprises introducing an insertion, deletion, substitution, duplication, or inversion of one or more nucleotides in the target nucleic acid of the cells of the population.

60. The method of any one of claims 57-59, wherein the gene is knocked down or knocked out.

61. The method of any one of claims 57-59, wherein the gene is modified such that a functional gene product can be expressed.

62. The method of any one of claims 57-61, wherein the rAAV comprises the first and the second gRNA, wherein the second gRNA comprises a targeting sequence complementary to a different target site in a gene targeted by the targeting sequence of the first gRNA, wherein the nucleotides between the target sites are excised by cleavage of the target sites by the CasX protein.

63. The method of any one of claims 57-61, wherein the rAAV comprises the first and the second gRNA, wherein the second gRNA comprises a targeting sequence complementary to a target site in a different gene targeted by the targeting sequence of the first gRNA, wherein the target nucleic acid at each target site is modified by the CasX protein.

64. A method of treating a disease in a subject caused by one or more mutations in a gene of the subject, comprising administering a therapeutically effective dose of the rAAV of any one of claims 40-56 to the subject.

65. The method of claim 62, wherein the rAAV is administered to the subject by a route of administration selected from subcutaneous, intradermal, intraneural, intranodal, intramedullary, intramuscular, intralumbar, intrathecal, subarachnoid, intraventricular, intracapsular, intravenous, intralymphatical, intraocular and intraperitoneal routes, and wherein the administration method is injection, transfusion, or implantation.

66. The method of claim 64 or claim 65, wherein the subject is selected from the group consisting of mouse, rat, pig, and non-human primate.

67. The method of claim 64 or claim 65, wherein the subject is a human.

68. A method of making a rAAV, comprising:
a. providing a population of packaging cells; and
b. transfecting the population of cells with:
i) a vector comprising the transgene of any one of claims 1-38;
ii) a vector comprising an Assembly-Activating Protein (AAP) gene; and
iii) a vector comprising rep and cap genomes.

69. The method of claim 68, wherein the packaging cell is selected from the group consisting of BHK cells, HEK293 cells, HEK293T cells, NSO cells, SP2/0 cells, YO myeloma cells, P3X63 mouse myeloma cells, PER cells, PER.C6 cells, hybridoma cells, NIH3T3 cells, COS cells, HeLa cells, and CHO cells.

70. The method of claim 68 or claim 69, the method further comprising recovering the rAAV.

71. The method of any one of claims 68-70, wherein the component sequences of the transgene are encompassed in a single recombinant adeno-associated virus particle.

72. A composition of a recombinant adeno-associated virus of any one of claims 40-56, for use in the manufacture of a medicament for the treatment of a disease in a human in need thereof.

73. A kit comprising the rAAV of any one of claim 40-56 and a suitable container.

74. The kit of claim 73, comprising a pharmaceutically acceptable carrier, diluent, buffer, or excipient.
